(19) United States
(12) Patent Application Publication
(10) Pub. No.: US 2023/0119114 A1
(43) Pub. Date: Apr. 20, 2023

(54) SOLID FORMS OF APOL1 INHIBITORS AND METHODS OF USING SAME

(71) Applicant: Vertex Pharmaceuticals Incorporated, Boston, MA (US)

(72) Inventors: Kevin Brett DANIEL, Boston, MA (US); Lori Ann FERRIS, Wilmington, MA (US); Kevin James GAGNON, Acton, MA (US); Michael David JUDELSON, Somerville, MA (US); Mei-Hsiu LAI, Waltham, MA (US); Courtney K. MAGUIRE, Watertown, MA (US); Ales MEDEK, Winchester, MA (US); Andrey PERESYPKIN, Newton, MA (US); Peter Jamison ROSE, Littleton, MA (US); Yi SHI, Natick, MA (US); Charlene TSAY, Boston, MA (US)

(21) Appl. No.: 17/895,582

(22) Filed: Aug. 25, 2022

Related U.S. Application Data
(60) Provisional application No. 63/237,248, filed on Aug. 26, 2021.
(60) Provisional application No. 63/306,831, filed on Feb. 4, 2022.
(60) Provisional application No. 63/315,936, filed on Mar. 2, 2022.

(51) Int. Cl. C07D 495/20; B01J 23/46

(52) U.S. Cl. CPC C07D 495/20; B01J 23/464

(57) ABSTRACT

The disclosure provides novel solid state forms of Compound I chosen from Compound I Phosphate Salt Hydrate Form A, Compound I free form Monohydrate, Compound I Phosphate Salt Methanol Solvate, and Compound I Phosphate Salt MEK Solvate, compositions comprising the same, and methods of making and using the same, including uses in treating APOL1 mediated disease (such as, e.g., APOL1 mediated kidney disease). Also provided herein are novel solid state forms of Compound II chosen from Compound II Phosphate Salt Hemihydrate Form A, Compound II free form Hemihydrate Form A, and Compound II free form Form C, compositions comprising the same, and methods of making and using the same, including uses in treating APOL1 mediated disease (such as, e.g., APOL1 mediated kidney disease).

This application claims the benefit of U.S. Provisional Application No. 63/237,248, filed on Aug. 26, 2021, U.S. Provisional Application No. 63/306,831, filed on Feb. 4, 2022, and U.S. Provisional Application No. 63/315,936, filed Mar. 2, 2022, the contents of which are incorporated by reference in their entirety.

This disclosure provides solid forms of compounds that may inhibit apolipoprotein L1 (APOL1) and methods of using those solid forms to treat APOL1-mediated diseases, such as, e.g., pancreatic cancer, APOL1 mediated kidney disease, including focal segmental glomerulosclerosis (FSGS), and/or non-diabetic kidney disease (NDKD). In some embodiments, the FSGS and/or NDKD is associated with common APOL1 genetic variants (G1: S342G:I384M and G2: N388del:Y389del). In some embodiments, the pancreatic cancer is associated with elevated levels of APOL1 (such as, e.g., elevated levels of APOL1 in pancreatic cancer tissues).

FSGS is a rare kidney disease with an estimated global incidence of 0.2 to 1.1/100,000/year. FSGS is a disease of the podocyte (glomerular visceral epithelial cells) responsible for proteinuria and progressive decline in kidney function. NDKD is a kidney disease involving damage to the podocyte or glomerular vascular bed that is not attributable to diabetes. NDKD is a disease characterized by hypertension and progressive decline in kidney function. Human genetics support a causal role for the G1 and G2 APOL1 variants in inducing kidney disease. Individuals with 2 APOL1 alleles are at increased risk of developing end-stage kidney disease (ESKD), including primary (idiopathic) FSGS, human immunodeficiency virus (HIV)-associated FSGS, NDKD, arterionephrosclerosis, lupus nephritis, microalbuminuria, and chronic kidney disease. See, P. Dummer et al., Semin Nephrol. 35(3): 222-236 (2015).

FSGS and NDKD can be divided into different subgroups based on the underlying etiology. One homogeneous subgroup of FSGS is characterized by the presence of independent common sequence variants in the apolipoprotein L1 (APOL1) gene termed G1 and G2, which are referred to as the “APOL1 risk alleles.” G1 encodes a correlated pair of non-synonymous amino acid changes (S342G and 1384M), G2 encodes a 2 amino acid deletion (N388del:Y389del) near the C terminus of the protein, and G0 is the ancestral (low risk) allele. A distinct phenotype of NDKD is found in patients with APOL1 genetic risk variants as well. In both APOL1-mediated FSGS and NDKD, higher levels of proteinuria and a more accelerated loss of kidney function occur in patients with two risk alleles compared to patients with the same disease who have no or just 1 APOL1 genetic risk variant. Alternatively, in AMKD, higher levels of proteinuria and accelerated loss of kidney function can also occur in patients with one risk allele. See, G. Vajgel et al., J. Rheumatol., November 2019, jrheum.190684.

APOL1 is a 44 kDa protein that is only expressed in humans, gorillas, and baboons. The APOL1 gene is expressed in multiple organs in humans, including the liver and kidney. APOL1 is produced mainly by the liver and contains a signal peptide that allows for secretion into the bloodstream, where it circulates bound to a subset of high-density lipoproteins. APOL1 is responsible for protection against the invasive parasite, Trypanosoma brucei brucei (T. b. brucei). APOL1 is endocytosed by T. b. brucei and transported to lysosomes, where it inserts into the lysosomal membrane and forms pores that lead to parasite swelling and death.

While the ability to lyse T. b. brucei is shared by all 3 APOL1 variants (G0, G1, and G2), APOL1 G1 and G2 variants confer additional protection against parasite species that have evolved a serum resistant associated-protein (SRA) which inhibits APOL1 G0; APOL1 G1 and G2 variants confer additional protection against trypanosoma species that cause sleeping sickness. G1 and G2 variants evade inhibition by SRA; G1 confers additional protection against T. b. gambiense (which causes West African sleeping sickness) while G2 confers additional protection against T. b. rhodesiense (which causes East African sleeping sickness).

In the kidney, APOL1 is expressed in podocytes, endothelial cells (including glomerular endothelial cells), and some tubular cells. Podocyte-specific expression of APOL1 G1 or G2 (but not G0) in transgenic mice induces structural and functional changes, including albuminuria, decreased kidney function, podocyte abnormalities, and glomerulosclerosis. Consistent with these data, G1 and G2 variants of APOL1 play a causative role in inducing FSGS and accelerating its progression in humans. Individuals with APOL1 risk alleles (i.e., homozygous or compound heterozygous for the APOL1 G1 or APOL1 G2 alleles) have increased risk of developing FSGS and they are at risk for rapid decline in kidney function if they develop FSGS. Thus, inhibition of APOL1 could have a positive impact in individuals who harbor APOL1 risk alleles.

Although normal plasma concentrations of APOL1 are relatively high and can vary at least 20-fold in humans, circulating APOL1 is not causally associated with kidney disease. However, APOL1 in the kidney is thought to be responsible for the development of kidney diseases, including FSGS and NDKD. Under certain circumstances, APOL1 protein synthesis can be increased by approximately 200-fold by pro-inflammatory cytokines such as interferons or tumor necrosis factor-α. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of end stage kidney disease (ESKD) is substantially higher in people of recent sub-Saharan African ancestry as compared to those of European ancestry. In the United States, ESKD is responsible for nearly as many lost years of life in women as from breast cancer and more lost years of life in men than from colorectal cancer.

FSGS and NDKD are caused by damage to podocytes, which are part of the glomerular filtration barrier, resulting in proteinuria. Patients with proteinuria are at a higher risk of developing ESKD and developing proteinuria-related complications, such as infections or thromboembolic events. There is no standardized treatment regimen nor approved drugs for FSGS or NDKD. Currently, FSGS and NDKD are managed with symptomatic treatment (including blood pressure control using blockers of the renin angiotensin system), and patients with FSGS and heavy proteinuria may be offered high dose steroids. Current therapeutic options for NDKD are anchored on blood pressure control and blockade of the renin angiotensin system.

Corticosteroids, alone or in combination with other immunosuppressants, induce remission in a minority of patients (e.g., remission of proteinuria in a minority of patients) and are associated with numerous side effects. However, remission is frequently indurable even in patients initially responsive to corticosteroid and/or immunosuppressant treatment. As a result, patients, in particular individuals of recent sub-Saharan African ancestry with 2 APOL1 risk alleles, experience rapid disease progression leading to end-stage renal disease (ESRD). Thus, there is an unmet medical need for treatment for FSGS and NDKD. Illustratively, in view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

Additionally, APOL1 is an aberrantly expressed gene in multiple cancers (Lin et al., Cell Death and Disease (2021), 12:760). Recently, APOL1 was found to be abnormally elevated in human pancreatic cancer tissues compared with adjacent tissues and was associated with poor prognosis in pancreatic cancer patients. In in vivo and in vitro experiments, knockdown of APOL1 significantly inhibited cancer cell proliferation and promoted the apoptosis of pancreatic cancer cells.

Compound I, its method of preparation, and physicochemical data are disclosed as Compound 181 in International Application No. PCT/US2021/047754, filed on Aug. 26, 2021, the entirety of which is incorporated herein by reference.

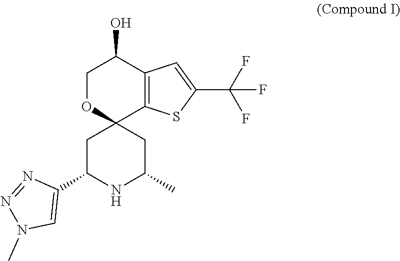 [SMILES: C[C@H]1C[C@@]2(C[C@@H](c3cn(C)nn3)N1)OC[C@@H](O)c1cc(C(F)(F)F)sc12]

Compound II, its method of preparation, and physicochemical data are disclosed as Compound 174 in International Application No. PCT/US2021/047754, filed on Aug. 26, 2021, the entirety of which is incorporated herein by reference.

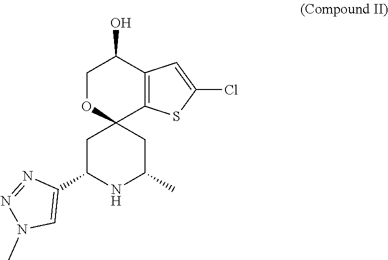 [SMILES: C[C@H]1C[C@@]2(C[C@@H](c3cn(C)nn3)N1)OC[C@@H](O)c1cc(Cl)sc12]

One aspect of the disclosure provides a new solid state form, Compound I Phosphate Salt Hydrate Form A, which can be employed in the treatment of diseases mediated by APOL1, such as FSGS, NDKD, and pancreatic cancer, and methods of making the same.

One aspect of the disclosure provides a new solid state form, Compound I free form Monohydrate, which can be employed in the treatment of diseases mediated by APOL1, such as FSGS, NDKD, and pancreatic cancer, and methods of making the same.

Another aspect of the disclosure provides a new solid state form, Compound I Maleate Form A (salt or co-crystal), which can be employed in the treatment of diseases mediated by APOL1, such as FSGS, NDKD, and pancreatic cancer, and methods of making the same.

Another aspect of the disclosure provides a new solid state form, Compound I Maleate Form B (salt or co-crystal), which can be employed in the treatment of diseases mediated by APOL1, such as FSGS, NDKD, and pancreatic cancer, and methods of making the same.

Another aspect of the disclosure provides a new solid state form, Compound I Fumaric Acid Form A (salt or co-crystal), which can be employed in the treatment of diseases mediated by APOL1, such as FSGS, NDKD, and pancreatic cancer, and methods of making the same.

Another aspect of the disclosure provides a new solid state form, Compound I free form Form B, which can be employed in the treatment of diseases mediated by APOL1, such as FSGS, NDKD, and pancreatic cancer, and methods of making the same.

Another aspect of the disclosure provides a new solid state form, Compound I free form Form C, which can be employed in the treatment of diseases mediated by APOL1, such as FSGS, NDKD, and pancreatic cancer, and methods of making the same.

Another aspect of the disclosure provides new solid state forms, Compound I Phosphate Salt Methanol Solvate and Compound I Phosphate Salt MEK Solvate, which can be employed in the manufacture of therapeutic solid forms of Compound I.

Another aspect of the disclosure provides a new solid state form, Compound II Phosphate Salt Hemihydrate Form A, which can be employed in the treatment of diseases mediated by APOL1, such as FSGS, NDKD, and pancreatic cancer, and methods of making the same.

Another aspect of the disclosure provides a new solid state form, Compound II free form Hemihydrate Form A, which can be employed in the treatment of diseases mediated by APOL1, such as FSGS, NDKD, and pancreatic cancer, and methods of making the same.

Another aspect of the disclosure provides a new solid state form, Compound II free form Form C, which can be employed in the treatment of diseases mediated by APOL1, such as FSGS, NDKD, and pancreatic cancer, and methods of making the same.

One aspect of the disclosure provides a new solid state form, Compound II free form Form A, which can be employed in the treatment of diseases mediated by APOL1, such as FSGS, NDKD, and pancreatic cancer, and methods of making the same.

One aspect of the disclosure provides a new solid state form, Compound II free form Form B, which can be employed in the treatment of diseases mediated by APOL1, such as FSGS, NDKD, and pancreatic cancer, and methods of making the same.

One aspect of the disclosure provides a new solid state form, Compound II free form Quarter Hydrate, which can be employed in the treatment of diseases mediated by APOL1, such as FSGS, NDKD, and pancreatic cancer, and methods of making the same.

One aspect of the disclosure provides a new solid state form, Compound II free form Hydrate Mixture, which can be employed in the treatment of diseases mediated by APOL1, such as FSGS, NDKD, and pancreatic cancer, and methods of making the same.

One aspect of the disclosure provides a new solid state form, Compound II free form Monohydrate, which can be employed in the treatment of diseases mediated by APOL1, such as FSGS, NDKD, and pancreatic cancer, and methods of making the same.

One aspect of the disclosure provides a new solid state form, Compound II free form Dihydrate, which can be employed in the treatment of diseases mediated by APOL1, such as FSGS, NDKD, and pancreatic cancer, and methods of making the same.

One aspect of the disclosure provides a new solid state form, Compound II free form EtOH Solvate Form B, which can be employed in the treatment of diseases mediated by APOL1, such as FSGS, NDKD, and pancreatic cancer, and methods of making the same.

One aspect of the disclosure provides a new solid state form, Compound II Phosphate Salt Form A, which can be employed in the treatment of diseases mediated by APOL1, such as FSGS, NDKD, and pancreatic cancer, and methods of making the same.

One aspect of the disclosure provides a new solid state form, Compound II Phosphate Salt Form C, which can be employed in the treatment of diseases mediated by APOL1, such as FSGS, NDKD, and pancreatic cancer, and methods of making the same.

One aspect of the disclosure provides Amorphous free form Compound II, which can be employed in the treatment of diseases mediated by APOL1, such as FSGS, NDKD, and pancreatic cancer, and methods of making the same.

Another aspect of the disclosure provides new solid state forms of Compound II, including, Compound II free form MEK Solvate, Compound II free form IPA Solvate, Compound II free form MeOH Solvate, and Compound II Phosphate Salt Acetone Solvate Form A, which can be employed in the manufacture of therapeutic solid forms of Compound II.

Another aspect of the disclosure provides methods of treating a APOL1-mediated disease (such as, e.g., pancreatic cancer, FSGS, and/or NDKD) comprising administering to a subject in need thereof, a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C, or a pharmaceutical composition comprising the same.

In some embodiments, the subject has 1 APOL1 risk allele. In some embodiments, the subject has 2 APOL1 risk alleles.

In some embodiments, the methods of treatment include administration of at least one additional active agent to the subject in need thereof, either in the same pharmaceutical composition as the solid form of Compound I, or as separate compositions.

In some embodiments, the solid form of Compound I and the at least one additional active agent are co-administered in the same pharmaceutical composition. In some embodiments, the solid form of Compound I and the at least one additional active agent are co-administered in separate pharmaceutical compositions. In some embodiments, the solid form of Compound I and the at least one additional active agent are co-administered simultaneously. In some embodiments, the solid form of Compound I and the at least one additional active agent are co-administered sequentially.

Another aspect of the disclosure provides methods of treating a APOL1-mediated disease (such as, e.g., pancreatic cancer, FSGS, and/or NDKD) comprising administering to a subject in need thereof, a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Compound II Phosphate Salt Form A, and Compound II Phosphate Salt Form C or a pharmaceutical composition comprising the same.

In some embodiments, the subject has 1 APOL1 risk allele. In some embodiments, the subject has 2 APOL1 risk alleles.

In some embodiments, the methods of treatment include administration of at least one additional active agent to the subject in need thereof, either in the same pharmaceutical composition as the solid form of Compound II, or as separate compositions.

In some embodiments, the solid form of Compound II and the at least one additional active agent are co-administered in the same pharmaceutical composition. In some embodiments, the solid form of Compound II and the at least one additional active agent are co-administered in separate pharmaceutical compositions. In some embodiments, the solid form of Compound II and the at least one additional active agent are co-administered simultaneously. In some embodiments, the solid form of Compound II and the at least one additional active agent are co-administered sequentially.

Also provided are methods of inhibiting APOL1, comprising administering to a subject in need thereof, a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C, or a pharmaceutical composition comprising the same.

Also provided are methods of inhibiting APOL1, comprising administering to a subject in need thereof, a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Compound II Phosphate Salt Form A, and Compound II Phosphate Salt Form C, or a pharmaceutical composition comprising the same.

Also disclosed herein is a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C for use in therapy. In some embodiments, the solid form of Compound I is combined with at least one additional active agent for simultaneous, separate, or sequential use in therapy. In some embodiments, when the use is simultaneous, the solid form of Compound I and the at least one additional active agent are in separate pharmaceutical compositions. In some embodiments, when the use is simultaneous, the solid form of Compound I and the at least one additional active agent are together in the same pharmaceutical composition.

Also disclosed herein is a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Amorphous free form Compound II, Compound II free form EtOH Solvate Form B, Compound II Phosphate Salt Form A, and Compound II Phosphate Salt Form C, for use in therapy. In some embodiments, the solid form of Compound II is combined with at least one additional active agent for simultaneous, separate, or sequential use in therapy. In some embodiments, when the use is simultaneous, the solid form of Compound II and the at least one additional active agent are in separate pharmaceutical compositions. In some embodiments, when the use is simultaneous, the solid form of Compound II and the at least one additional active agent are together in the same pharmaceutical composition.

Also disclosed herein is a pharmaceutical composition comprising a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C for use in therapy.

Also disclosed herein is a pharmaceutical composition comprising a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Amorphous free form Compound II, Compound II free form EtOH Solvate Form B, Compound II Phosphate Salt Form A, and Compound II Phosphate Salt Form C for use in therapy.

It should be understood that references herein to methods of treatment and/or inhibition (e.g., methods of treating FSGS and/or NDKD; methods of inhibiting APOL1) using one or more compounds (e.g., one or more solid forms of Compound I or Compound II as described herein) should also be interpreted as references to:

one or more compounds (e.g., one or more solid forms of Compound I or Compound II), for use in methods of treatment and/or inhibition; and/or

the use of one or more compounds (e.g., one or more solid forms of Compound I or Compound II) in the manufacture of a medicament for treatment and/or inhibition.

### BRIEF DESCRIPTION OF THE DRAWINGS

FIG. 1 depicts an XRPD diffractogram of Compound I Phosphate Salt Methanol Solvate.

FIG. 2 depicts a solid state ¹³C NMR spectrum of Compound I Phosphate Salt Methanol Solvate.

FIG. 3 depicts a solid state ¹⁹F NMR spectrum of Compound I Phosphate Salt Methanol Solvate.

FIG. 4 depicts a solid state ³¹P NMR spectrum of Compound I Phosphate Salt Methanol Solvate.

FIG. 5 depicts an XRPD diffractogram of Compound I Phosphate Salt Hydrate Form A at 25±2° C. and 40% RH.

FIG. 6 depicts an XRPD diffractogram of Compound I Phosphate Salt Hydrate Form A at 25±2° C. and 5% RH (black trace) or 90% RH (gray trace).

FIG. 7 depicts a solid state ¹³C NMR spectrum of Compound I Phosphate Salt Hydrate Form A at 43% RH.

FIG. 8 depicts a solid state ¹⁹F NMR spectrum of Compound I Phosphate Salt Hydrate Form A at 43% RH.

FIG. 9 depicts the effects of relative humidity on solid state ¹⁹F NMR spectrum of Compound I Phosphate Salt Hydrate Form A.

FIG. 10 depicts a solid state ³¹P NMR spectrum of Compound I Phosphate Salt Hydrate Form A at 43% RH.

FIG. 11 depicts the effects of relative humidity on solid state ³¹P NMR spectrum of Compound I Phosphate Salt Hydrate Form A.

FIG. 12 depicts a TGA thermogram of Compound I Phosphate Salt Hydrate Form A.

FIG. 13 depicts a DSC curve of Compound I Phosphate Salt Hydrate Form A.

FIG. 14 depicts an XRPD diffractogram of Compound I free form Monohydrate.

FIG. 15 depicts a solid state ¹³C NMR spectrum of Compound I free form Monohydrate.

FIG. 16 depicts a solid state ¹³C NMR spectrum of dehydrated Compound I free form Monohydrate.

FIG. 17 depicts a solid state ¹⁹F NMR spectrum of Compound I free form Monohydrate.

FIG. 18 depicts a solid state ¹⁹F NMR spectrum of dehydrated Compound I free form Monohydrate.

FIG. 19 depicts a TGA thermogram of Compound I free form Monohydrate.

FIG. 20 depicts a DSC curve of Compound I free form Monohydrate.

FIG. 21 depicts an XRPD diffractogram of Compound I Phosphate Salt MEK Solvate.

FIG. 22 depicts a solid state ¹³C NMR spectrum of Compound I Phosphate Salt MEK Solvate.

FIG. 23 depicts a solid state ¹⁹F NMR spectrum of Compound I Phosphate Salt MEK Solvate.

FIG. 24 depicts an XRPD diffractogram of Compound II Phosphate Salt Hemihydrate Form A.

FIG. 25 depicts a solid state ¹³C NMR spectrum of Compound II Phosphate Salt Hemihydrate Form A.

FIG. 26 depicts a solid state ¹³C NMR spectrum of dehydrated Compound II Phosphate Salt Hemihydrate Form A.

FIG. 27A depicts a solid state ³¹P NMR spectrum of Compound II Phosphate Salt Hemihydrate Form A.

FIG. 27B depicts a solid state ³¹P NMR spectrum of dehydrated Compound II Phosphate Salt Hemihydrate Form A.

FIG. 28 depicts a TGA thermogram of Compound II Phosphate Salt Hemihydrate Form A.

FIG. 29 depicts a DSC curve of Compound II Phosphate Salt Hemihydrate Form A.

FIG. 30A depicts an XRPD diffractogram of Compound II free form Hemihydrate Form A measured at ambient temperature (25±2° C.).

FIG. 30B depicts an XRPD diffractogram of Compound II free form Hemihydrate Form A measured at a temperature between 40° C. and 50° C.

FIG. 30C depicts an XRPD diffractogram of Compound II free form Hemihydrate Form A measured at a temperature between 60° C. and 90° C.

FIG. 31 depicts a solid state ¹³C NMR spectrum of Compound II free form Hemihydrate Form A.

FIG. 32 is intentionally left blank.

FIG. 33 depicts a TGA thermogram of Compound II free form Hemihydrate Form A.

FIG. 34 depicts a DSC curve of Compound II free form Hemihydrate Form A.

FIG. 35 depicts an XRPD diffractogram of Compound II free form Form C measured at room temperature (25° C.±2° C.).

FIG. 36 depicts a TGA thermogram of Compound II free form Form C.

FIG. 37 depicts a DSC curve of Compound II free form Form C.

FIG. 38 depicts a solid state ¹³C NMR spectrum of Compound II free form Form C.

FIG. 39 depicts an XRPD diffractogram of Compound I Maleate Form A.

FIG. 40 depicts a TGA thermogram of Compound I Maleate Form A.

FIG. 41 depicts a DSC curve of Compound I Maleate Form A.

FIG. 42 depicts an XRPD diffractogram of Compound I Maleate Form B.

FIG. 43 depicts a TGA thermogram of Compound I Maleate Form B.

FIG. 44 depicts a DSC curve of Compound I Maleate Form B.

FIG. 45 depicts an XRPD diffractogram of Compound I Fumaric Acid Form A.

FIG. 46 depicts a solid state ¹³C CPMAS spectrum of Compound I Fumaric Acid Form A.

FIG. 47 depicts a solid state ¹⁹F MAS spectrum of Compound I Fumaric Acid Form A.

FIG. 48 depicts a TGA thermogram of Compound I Fumaric Acid Form A.

FIG. 49 depicts a DSC curve of Compound I Fumaric Acid Form A.

FIG. 50 depicts an XRPD diffractogram of Compound I free form Form B.

FIG. 51 depicts a solid state ¹³C CPMAS spectrum of Compound I free form Form B.

FIG. 52 depicts a solid state ¹⁹F MAS spectrum of Compound I free form Form B.

FIG. 53 depicts a TGA thermogram of Compound I free form Form B.

FIG. 54 depicts a DSC curve of Compound I free form Form B.

FIG. 55 depicts an XRPD diffractogram of Compound I free form Form C.

FIG. 56 depicts a solid state ¹³C CPMAS spectrum of Compound I free form Form C.

FIG. 57 depicts a solid state ¹⁹F MAS spectrum of Compound I free form Form C.

FIG. 58 depicts a TGA thermogram of Compound I free form Form C.

FIG. 59 depicts a DSC curve of Compound I free form free form Form C.

FIG. 60 depicts an XRPD diffractogram of Compound II free form Form A.

FIG. 61 depicts a solid state ¹³C CPMAS spectrum of Compound II free form Form A.

FIG. 62 depicts a TGA thermogram of Compound II free form Form A.

FIG. 63 depicts a DSC curve of Compound II free form Form A.

FIG. 64 depicts a solid state ¹³C CPMAS spectrum of Compound II free form Form B.

FIG. 65 depicts a solid state ¹³C CPMAS spectrum of a physical mixture of Compound II free form Quarter Hydrate with about 19% Compound II free form Hemihydrate Form A.

FIG. 66 depicts a solid state ¹³C CPMAS spectrum of Compound II free form Quarter Hydrate with the spectrum for Compound II free form Hemihydrate Form A subtracted.

FIG. 67 depicts an XRPD diffractogram of Compound II free form Hydrate Mixture.

FIG. 68 depicts a solid state ¹³C CPMAS spectrum of Compound II free form Monohydrate.

FIG. 69 depicts a solid state ¹³C CPMAS spectrum of Compound II free form Dihydrate mixed with about 29% Compound II free form Hemihydrate Form A and about 18% Compound II free form A.

FIG. 70 depicts a solid state ¹³C CPMAS spectrum of Compound II free form Dihydrate (spectra of 29% Compound II free form Hemihydrate Form A and about 18% Compound II free form A subtracted).

FIG. 71 depicts an XRPD diffractogram of Compound II free form EtOH Solvate Form B.

FIG. 72 depicts a TGA thermogram of Compound II free form EtOH Solvate Form B.

FIG. 73 depicts a DSC curve of Compound II free form EtOH Solvate Form B.

FIG. 74 depicts an XRPD diffractogram of Compound II free form IPA Solvate.

FIG. 75 depicts a solid state ¹³C CPMAS spectrum of Compound II free form IPA Solvate.

FIG. 76 depicts a solid state ¹³C CPMAS spectrum of Compound II free form MEK Solvate.

FIG. 77 depicts an XRPD diffractogram of Compound II free form MeOH Solvate.

FIG. 78 depicts a solid state ¹³C CPMAS spectrum of Compound II free form MeOH Solvate.

FIG. 79 depicts a TGA thermogram of Compound II free form MeOH Solvate.

FIG. 80 depicts a DSC curve of Compound II free form MeOH Solvate.

FIG. 81 depicts an XRPD diffractogram of Amorphous free form Compound II.

FIG. 82 depicts a solid state ¹³C CPMAS spectrum of Amorphous free form Compound II.

FIG. 83 depicts a TGA thermogram of Amorphous free form Compound II.

FIG. 84 depicts a DSC curve of Amorphous free form Compound II.

FIG. 85 depicts an XRPD diffractogram of Compound II Phosphate Salt Acetone Solvate Form A.

FIG. 86 depicts a solid state ¹³C CPMAS spectrum of Compound II Phosphate Salt Acetone Solvate Form A.

FIG. 87 depicts a TGA thermogram of Compound II Phosphate Salt Acetone Solvate Form A.

FIG. 88 depicts a DSC curve of Compound II Phosphate Salt Acetone Solvate Form A.

FIG. 89 depicts an XRPD diffractogram of Compound II Phosphate Salt Form A.

FIG. 90 depicts a solid state ¹³C CPMAS spectrum of Compound II Phosphate Salt Form A.

FIG. 91 depicts a solid state ³¹P CPMAS spectrum of Compound II Phosphate Salt Form A.

FIG. 92 depicts a TGA thermogram of Compound II Phosphate Salt Form A.

FIG. 93 depicts a DSC curve of Compound II Phosphate Salt Form A.

FIG. 94 depicts an XRPD diffractogram of Compound II Phosphate Salt Form C.

FIG. 95 depicts a solid state ¹³C CPMAS spectrum of Compound II Phosphate Salt Form C.

FIG. 96 depicts a TGA thermogram of Compound II Phosphate Salt Form C.

FIG. 97 depicts a DSC curve of Compound II Phosphate Salt Form C.

FIG. 98 depicts an XRPD diffractogram of Compound I Phosphate Salt Form B.

FIG. 99 depicts a TGA thermogram of Compound I Phosphate Salt Form B.

FIG. 100 depicts a DSC curve of Compound I Phosphate Salt Form B.

FIG. 101 depicts a solid state ¹³C NMR spectrum of Compound I Phosphate Salt Form B.

FIG. 102 depicts a solid state ¹⁹F NMR spectrum of Compound I Phosphate Salt Form B.

FIG. 103 depicts a solid state ³¹P NMR spectrum of Compound I Phosphate Salt Form B.

FIG. 104 depicts an XRPD diffractogram of Compound I Phosphate Salt Form C.

FIG. 105 depicts a TGA thermogram of Compound I Phosphate Salt Form C.

FIG. 106 depicts a DSC curve of Compound I Phosphate Salt Form C.

FIG. 107 depicts a solid state ¹³C NMR spectrum of Compound I Phosphate Salt Form C.

FIG. 108 depicts a solid state ¹⁹F NMR spectrum of Compound I Phosphate Salt Form C.

FIG. 109 depicts a solid state ³¹P NMR spectrum of Compound I Phosphate Salt Form C.

FIG. 110 depicts an XRPD diffractogram of Compound I Phosphate Salt Crystalline Form Mixture.

FIG. 111 depicts a TGA thermogram of Compound I Phosphate Salt Crystalline Form Mixture.

FIG. 112 depicts a DSC curve of Compound I Phosphate Salt Crystalline Form Mixture.

FIG. 113 depicts a solid state ¹³C NMR spectrum of Compound I Phosphate Salt Crystalline Form Mixture.

FIG. 114 depicts a solid state ¹⁹F NMR spectrum of Compound I Phosphate Salt Crystalline Form Mixture.

FIG. 115 depicts a solid state ³¹P NMR spectrum of Compound I Phosphate Salt Crystalline Form Mixture.

### DEFINITIONS

The term “APOL1,” as used herein, means apolipoprotein L1 protein, and the term “APOL1” means apolipoprotein L1 gene.

The term “APOL1 mediated disease” refers to a disease or condition associated with aberrant APOL1 (e.g., certain APOL1 genetic variants; elevated levels of APOL1). In some embodiments, an APOL1 mediated disease is an APOL1 mediated kidney disease. In some embodiments, an APOL1 mediated disease is associated with patients having two APOL1 risk alleles, e.g., patients who are homozygous or compound heterozygous for the G1 or G2 alleles. In some embodiments, an APOL1 mediated disease is associated with patients having one APOL1 risk allele.

The term “APOL1 mediated kidney disease” refers to a disease or condition that impairs kidney function and can be attributed to APOL1. In some embodiments, APOL1 mediated kidney disease is associated with patients having two APOL1 risk alleles, e.g., patients who are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease. In some embodiments, the APOL1 mediated kidney disease is chronic kidney disease or proteinuria.

The term “FSGS,” as used herein, means focal segmental glomerulosclerosis, which is a disease of the podocyte (glomerular visceral epithelial cells) responsible for proteinuria and progressive decline in kidney function. In some embodiments, FSGS is associated with two APOL1 risk alleles.

The term “NDKD,” as used herein, means non-diabetic kidney disease, which is characterized by severe hypertension and progressive decline in kidney function. In some embodiments, NDKD is associated with two APOL1 risk alleles.

The terms “ESKD” and “ESRD” are used interchangeably herein to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this disclosure will depend upon a number of factors, including the isotopic purity of reagents used to make the compound and the efficiency of incorporation of isotopes in the various synthesis steps used to prepare the compound. However, as set forth above, the relative amount of such isotopologues in total will be less than 49.9% of the compound. In other embodiments, the relative amount of such isotopologues in total will be less than 47.5%, less than 40%, less than 32.5%, less than 25%, less than 17.5%, less than 10%, less than 5%, less than 3%, less than 1%, or less than 0.5% of the compound.

The term “stable,” as used herein, refers to compounds or solid forms that are not substantially altered when subjected to conditions to allow for their production, detection, and preferably their recovery, purification, and use for one or more of the purposes disclosed herein.

The term “chemically stable,” as used herein, means that the solid form of Compound I or Compound II does not decompose into one or more different chemical compounds when subjected to specified conditions, e.g., 40° C./75% relative humidity, for a specific period of time, e.g., 1 day, 2 days, 3 days, 1 week, 2 weeks, or longer. In some embodiments, less than 25% of the solid form of Compound I or Compound II decomposes. In some embodiments, less than about 20%, less than about 15%, less than about 10%, less than about 5%, less than about 3%, less than about 1%, less than about 0.5% of the form of Compound I or Compound II decomposes under the conditions specified. In some embodiments, no detectable amount of the solid form of Compound I or Compound II decomposes.

The term “physically stable,” as used herein, means that the solid form of Compound I or Compound II does not change into one or more different physical forms of Compound I or Compound II (e.g., different solid forms as measured by XRPD, DSC, etc.) when subjected to specific conditions, e.g., 40° C./75% relative humidity, for a specific period of time, e.g, 1 day, 2 days, 3 days, 1 week, 2 weeks, or longer. In some embodiments, less than 25% of the solid form of Compound I or Compound II changes into one or more different physical forms when subjected to specified conditions. In some embodiments, less than about 20%, less than about 15%, less than about 10%, less than about 5%, less than about 3%, less than about 1%, less than about 0.5% of the solid form of Compound I or Compound II changes into one or more different physical forms of Compound I or Compound II when subjected to specified conditions. In some embodiments, no detectable amount of the solid form of Compound I or Compound II changes into one or more physically different solid forms of Compound I or Compound II.

As used herein, the term “hydrate” refers to any crystalline Compound I or crystalline Compound II that contains water in its crystal lattice. The stoichiometry of a Compound I hydrate or a Compound II hydrate can vary. For example, a hydrate of Compound I or of Compound II can be a quarter hydrate, hemihydrate, monohydrate, dihydrate, or a partially dehydrated form.

A “free base” form of a compound does not contain an ionically bonded salt. It is noted that the disclosed amounts of the compounds or their pharmaceutically acceptable salts thereof herein are based upon their free base form. For example, “10 mg of at least one compound chosen from Compound I and pharmaceutically acceptable salts thereof” includes 10 mg of Compound I and a mass of a pharmaceutically acceptable salt of Compound I equivalent to 10 mg of Compound I.

“Selected from” and “chosen from” are used interchangeably herein.

As used herein, the term “solvent” refers to any liquid in which the product is at least partially soluble (solubility of product >1 g/L).

Non-limiting examples of suitable solvents that may be used in methods of this disclosure include water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of amine bases that may be used in this disclosure include, for example, 1,8-diazabicyclo[5.4.0]undec-7-ene (DBU), N-methylmorpholine (NMM), triethylamine (Et₃N; TEA), diisopropylethyl amine (i-Pr₂EtN; DIPEA), pyridine, 2,2,6,6-tetramethylpiperidine, 1,5,7- triazabicyclo[4.4.0]dec-5-ene (TBD), 7-methyl-1,5,7-triazabicyclo[4.4.0]dec-5-ene (MTBD), t-Bu-tetramethylguanidine, pyridine, 1,5-diazabicyclo[4.3.0]non-5-ene (DBN), and potassium bis(trimethylsilyl)amide (KHMDS).

Non-limiting examples of carbonate bases that may be used in this disclosure include, for example, sodium carbonate (Na₂CO₃), potassium carbonate (K₂CO₃), cesium carbonate (Cs₂CO₃), lithium carbonate (Li₂CO₃), sodium bicarbonate (NaHCO₃), and potassium bicarbonate (KHCO₃).

Non-limiting examples of alkoxide bases that may be used in this disclosure include, for example, t-AmOLi (lithium t-amylate), t-AmONa (sodium t-amylate), t-AmOK (potassium t-amylate), sodium tert-butoxide (NaOtBu), potassium tert-butoxide (KOtBu), and sodium methoxide (NaOMe; NaOCH₃).

Non-limiting examples of hydroxide bases that may be used in this disclosure include, for example, lithium hydroxide (LiOH), sodium hydroxide (NaOH), and potassium hydroxide (KOH).

Non-limiting examples of phosphate bases that may be used in this disclosure include, for example, sodium phosphate tribasic (Na₃PO₄), potassium phosphate tribasic (K₃PO₄), potassium phosphate dibasic (K₂HPO₄), and potassium phosphate monobasic (KH₂PO₄).

Non-limiting examples of acids that may be used in this disclosure include, for example, trifluoroacetic acid (TFA), hydrochloric acid (HCl), methanesulfonic acid (MsOH), phosphoric acid (H₃PO₄), and sulfuric acid (H₂SO₄).

Non-limiting examples of organic acids that may be used in this disclosure include, for example, acetic acid, oxalic acid, maleic acid, tartaric acid, citric acid, succinic acid, and malonic acid.

Non-limiting examples of mineral acids that may be used in this disclosure include, for example, hydrochloric acid (HCl), nitric acid (HNO₃), phosphoric acid (H₃PO₄), hydrofluoric acid (HF), and sulfuric acid (H₂SO₄).

A non-limiting example of a carboxylic acid that may be used in this disclosure is trichloroacetic acid.

A non-limiting example of a phosphonic acid that may be used in this disclosure is phenylphosphonic acid.

Non-limiting examples of sulfonic acids that may be used in this disclosure include, for example, p-toluenesulfonic acid, benzenesulfonic acid, 4-(2-hydroxyethyl)-1-piperazineethanesulfonic acid (HEPES), and methanesulfonic acid.

Non-limiting examples of metal hydroxides that may be used in this disclosure include, for example, lithium hydroxide (LiOH), sodium hydroxide (NaOH), cesium hydroxide (CsOH), and potassium hydroxide (KOH).

Non-limiting examples of activating agents that may be used in this disclosure include, for example, carbonyl diimidazole, hydroxybenzotriazole (HOBt), and N,N-dimethylamino pyridine (DMAP).

Non-limiting examples of brominating agents that may be used in this disclosure include, for example, bromine (Br₂), N-bromosuccinimide (NBS), and 1,3-dibromo-5,5-dimethylhydantoin (DBDMH).

Non-limiting examples of phosphonium reagents that may be used in this disclosure include, for example, benzotriazole-1-yl-oxy-tris-(dimethylamino)-phosphonium hexafluorophosphate (BOP), benzotriazole-1-yl-oxy-tris-pyrrolidino-phosphonium hexafluorophosphate (PyBOP), 7-azabenzotriazol-1-yloxy)trispyrrolidinophosphonium hexafluorophosphate (PyAOP).

Non-limiting examples of peptide coupling reagents that may be used in this disclosure include, for example, N,N′-dicyclohexylcarbodiimide (DCC), 1-ethyl-3-(3-dimethylaminopropyl)carbodiimide hydrochloride, N-ethyl-N′-(3-dimethylaminopropyl)carbodiimide (EDCl), and 1-propanephosphonic anhydride (T3P).

Non-limiting examples of acetylating reagents that may be used in this disclosure include, for example, acetyl chloride, acetyl bromide, and acetic anhydride.

Non-limiting examples of iodinating reagents that may be used in this disclosure include, for example, iodine (I₂), N-iodosuccinimide (NIS), and 1,3-diiodo-5,5-dimethylhydantoin (DIH).

Non-limiting examples of uronium reagents that may be used in this disclosure include, for example, 1-[bis(dimethylamino)methylene]-1H-1,2,3-triazolo[4,5-b]pyridinium 3-oxid hexafluorophosphate (HATU), N,N,N′,N′-tetramethyl-O-(1H-benzotriazol-1-yl)uranium hexafluorophosphate (HBTU).

Non-limiting examples of trifluoromethylating reagents that may be used in this disclosure include, for example, (1,10-phenanthroline)(trifluoromethyl)copper(I).

Non-limiting examples of nucleophilc methyls that may be used in this disclosure include, for example, MeLi and MeMgBr.

As used herein, the terms “about” and “approximately,” when used in connection with amounts, volumes, reaction times, reaction temperatures, etc. mean an acceptable error for a particular value as determined by one of ordinary skill in the art, which depends in part on how the value is measured or determined. In some embodiments, the terms “about” and “approximately” mean within 1, 2, 3, or 4 standard deviations. In certain embodiments, the terms “about” and “approximately” mean within 20%, 15%, 10%, 9%, 8%, 7%, 6%, 5%, 4%, 3%, 2%, 1%, 0.5%, 0.1%, or 0.05% of a given value or range. As used herein, the symbol “˜” appearing immediately before a numerical value has the same meaning as the terms “about” and “approximately.”

The terms “patient” and “subject” are used interchangeably herein and refer to an animal, including a human. In some embodiments, the subject is a human.

The terms “effective dose” and “effective amount” are used interchangeably herein and refer to that amount of compound that produces a desired effect for which it is administered (e.g., improvement in one or more symptoms of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates, as used herein, include, but are not limited to, the following: eliminating or reducing the severity of any symptom, complete or partial remission, lower risk of kidney failure (e.g., ESRD), and disease-related complications (e.g., edema, susceptibility to infections, or thrombo-embolic events). Improvements in or lessening the severity of any of the symptoms of APOL1 mediated disease (e.g., APOL1 mediated kidney disease) can be readily assessed according to methods and techniques known in the art or subsequently developed. In some embodiments, the terms “treat,” “treating,” and “treatment” refer to the lessening of severity of one or more symptoms of FSGS and/or NDKD.

The solid forms of Compound I disclosed herein may be administered once daily, twice daily, or three times daily, for example, for the treatment of a APOL1 mediated disease (e.g., FSGS). In some embodiments, the solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is administered once daily. In some embodiments, the solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is administered twice daily. In some embodiments, the solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is administered three times daily.

In some embodiments, 2 mg to 1500 mg of the solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is administered once daily, twice daily, or three times daily.

The solid forms of Compound II disclosed herein may be administered once daily, twice daily, or three times daily, for example, for the treatment of a APOL1 mediated disease (e.g., FSGS). In some embodiments, the solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C, is administered once daily. In some embodiments, the solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C, is administered twice daily. In some embodiments, the solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C, is administered three times daily.

In some embodiments, 2 mg to 1500 mg of the solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Compound II free form IPA Solvate, Compound II free form MEK Solvate, Compound II free form MeOH Solvate, Amorphous free form Compound II, Compound II Phosphate Salt Acetone Solvate Form A, Compound II Phosphate Salt Form A, and Compound II Phosphate Salt Form C, is administered once daily, twice daily, or three times daily.

As used herein, the term “ambient conditions” means room temperature, open air, and uncontrolled humidity conditions. The terms “room temperature” and “ambient temperature” mean 15° C. to 30° C.

As used herein, the terms “crystalline form” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, solid state nuclear magnetic resonance (SSNMR), differential scanning calorimetry (DSC), infrared radiation (IR), and/or thermogravimetric analysis (TGA). Accordingly, as used herein, the term “crystalline Form [X] of Compound [Y]” refers to a unique crystalline form that can be identified and distinguished from other crystalline forms of Compound [Y] by one or more characterization techniques including, for example, X-ray powder diffraction (XRPD), single crystal X-ray diffraction, SSNMR, differential scanning calorimetry (DSC), infrared radiation (IR), and/or thermogravimetric analysis (TGA). In some embodiments, the novel crystalline Form [X] of Compound [Y] is characterized by an X-ray powder diffractogram having one or more signals at one or more specified two-theta values (° 20).

As used herein, the term “SSNMR” refers to the analytical characterization method of solid state nuclear magnetic resonance. SSNMR spectra can be recorded at ambient or non-ambient (e.g., at 275 K) conditions on any magnetically active isotope present in the sample. Common examples of active isotopes for small molecule active pharmaceutical ingredients include ¹H, ²H, ¹³C, ¹⁹F, ³¹P, ¹⁵N, ¹⁴N, ³⁵Cl, ¹¹B, ⁷Li, ¹⁷O, ²³Na, ⁷⁹Br, and ¹⁹⁵Pt.

As used herein, the term “XRPD” refers to the analytical characterization method of X-ray powder diffraction. XRPD patterns can be recorded under ambient conditions in transmission or reflection geometry using a diffractometer.

As used herein, the terms “X-ray powder diffractogram,” “X-ray powder diffraction pattern,” and “XRPD pattern” interchangeably refer to an experimentally obtained pattern plotting signal positions (on the abscissa) versus signal intensities (on the ordinate). For an amorphous material, an X-ray powder diffractogram may include one or more broad signals; and for a crystalline material, an X-ray powder diffractogram may include one or more signals, each identified by its angular value as measured in degrees 2θ (° 2θ), depicted on the abscissa of an X-ray powder diffractogram, which may be expressed as “a signal at . . . degrees two-theta,” “a signal at [a] two-theta value(s) of . . . ” and/or “a signal at at least . . . two-theta value(s) chosen from . . . .”

A “signal” or “peak,” as used herein, refers to a point in the XRPD pattern where the intensity as measured in counts is at a local maximum. One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

As used herein, “a signal at at . . . degrees two-theta,” “a signal at [a] two-theta value[ ] of . . . ,” and/or “a signal at at least . . . two-theta value(s) chosen from . . . ” refer to X-ray reflection positions as measured and observed in X-ray powder diffraction experiments (° 2θ).

The repeatability of the angular values is in the range of ±0.2° 2θ, i.e., the angular value can be at the recited angular value +0.2 degrees two-theta, the angular value −0.2 degrees two-theta, or any value between those two end points (angular value +0.2 degrees two-theta and angular value −0.2 degrees two-theta).

As used herein, the terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

The terms “X-ray powder diffractogram having a signal at . . . two-theta values” and “X-ray powder diffractogram comprising a signal at . . . two-theta values” are used interchangeably herein and refer to an XRPD pattern that contains X-ray reflection positions as measured and observed in X-ray powder diffraction experiments (° 2θ).

As used herein, an X-ray powder diffractogram is “substantially similar to that in [a particular] Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal positions in XRPD diffractograms (in degrees two-theta (° 2θ) referred to herein) generally mean that value reported is +0.2 degrees 2θ of the reported value, an art-recognized variance.

As used herein, an SSNMR spectrum is “substantially similar to that in [a particular] Figure” when at least 90%, such as at least 95%, at least 98%, or at least 99%, of the signals in the two spectra overlap. In determining “substantial similarity,” one of ordinary skill in the art will understand that there may be variation in the intensities and/or signal positions in SSNMR spectra even for the same crystalline form. Thus, those of ordinary skill in the art will understand that the signal positions in SSNMR spectra (in ppm) referred to herein generally mean that value reported is +0.2 ppm of the reported value, an art-recognized variance.

As used herein, a DSC curve is “substantially similar to that in [a particular] Figure” when at least 90%, such as at least 95%, at least 98%, or at least 99%, of the features in the two curves overlap. In determining “substantial similarity,” one of ordinary skill in the art will understand that there may be variation in the intensities and/or peak (e.g., endotherm or exotherm) positions in DSC curves, even for the same solid form.

As used herein, a TGA thermogram is “substantially similar to that in [a particular] Figure” when at least 90%, such as at least 95%, at least 98%, or at least 99%, of the features in the two thermograms overlap. In determining “substantial similarity,” one of ordinary skill in the art will understand that there may be variation in the intensities and/or peak (e.g., degradation peak) positions in TGA thermograms even for the same solid form.

As used herein, a crystalline form is “substantially pure” when it accounts for an amount by weight equal to or greater than 90% of the sum of all solid form(s) in a sample as determined by a method in accordance with the art, such as, e.g., quantitative XRPD. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

As used herein, the term “DSC” refers to the analytical method of Differential Scanning Calorimetry.

As used herein, the term “TGA” refers to the analytical method of Thermo Gravimetric (or thermogravimetric) Analysis.

As used herein, a “crystalline hydrate” is a crystal form comprising either stoichiometric or nonstoichiometric water in the crystal lattice. In the case of nonstoichiometric hydrate, the amount of water present in a crystalline hydrate may vary as a function of at least the relative humidity (“RH”). The presence (or absence) of water or different amounts of water may lead to X-ray diffractogram peak position shifts, or the appearance or disappearance of peaks. The presence (or absence) of water or different amount of water may lead to peak shifts or even appearances of new peaks in proton, carbon, fluorine, phosphorus, nitrogen, chlorine (or other NMR active nuclei) solid state NMR spectra.

Compound I is disclosed as Compound 181 in International Application No. PCT/US2021/047754, filed on Aug. 26, 2021, the entire contents of which are incorporated herein by reference.

Compound I is depicted as follows:

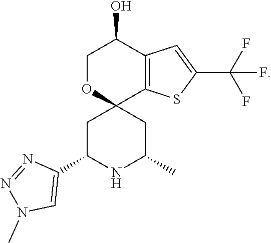 [SMILES: C[C@H]1C[C@@]2(C[C@@H](c3cn(C)nn3)N1)OC[C@@H](O)c1cc(C(F)(F)F)sc12]

Compound II is disclosed as Compound 174 in International Application No. PCT/US2021/047754, filed on Aug. 26, 2021, the entire contents of which are incorporated herein by reference.

Compound II is depicted as follows:

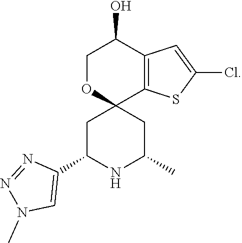 [SMILES: C[C@H]1C[C@@]2(C[C@@H](c3cn(C)nn3)N1)OC[C@@H](O)c1cc(Cl)sc12]

### Compound I Phosphate Salt Hydrate Form A

Some embodiments of the disclosure provide a phosphate salt hydrate of Compound I (Compound I Phosphate Salt Hydrate Form A). In some embodiments, the Compound I Phosphate Salt Hydrate Form A is substantially pure.

In some embodiments, the Compound I Phosphate Salt Hydrate Form A is characterized by an X-ray powder diffractogram comprising a signal at 8.6, 19.9, and/or 28.3±0.2 two-theta. In some embodiments, the Compound I Phosphate Salt Hydrate Form A is characterized by an X-ray powder diffractogram comprising a signal at the following two-theta values: 8.6±0.2, 19.9±0.2, and 28.3±0.2.

In some embodiments, the Compound I Phosphate Salt Hydrate Form A is characterized by an X-ray powder diffractogram comprising a signal at one or more (e.g., two or more) two-theta values chosen from 8.6±0.2, 19.9±0.2, and 28.3±0.2. In some embodiments, the Compound I Phosphate Salt Hydrate Form A is characterized by an X-ray powder diffractogram comprising a signal at two or more two-theta values chosen from 8.6±0.2, 19.9±0.2, and 28.3±0.2.

In some embodiments, the Compound I Phosphate Salt Hydrate Form A is characterized by an X-ray powder diffractogram measured at 25±2° C. and 5% relative humidity (RH) comprising (a) a signal at the following two-theta values: 8.6±0.2, 19.9±0.2, and 28.3±0.2; and (b) a signal at one or more (e.g., two or more) two-theta values chosen from 17.2±0.2, 20.4±0.2, and 22.8±0.2. In some embodiments, the Compound I Phosphate Salt Hydrate Form A is characterized by an X-ray powder diffractogram measured at 25±2° C. and 5% relative humidity (RH) comprising a signal at one or more two-theta values (e.g., two or more, three or more, four or more, five or more) chosen from 8.6±0.2, 17.2±0.2, 19.9±0.2, 20.4±0.2, 22.8±0.2, and 28.3±0.2. In some embodiments, the Compound I Phosphate Salt Hydrate Form A is characterized by an X-ray powder diffractogram measured at 25±2° C. and 5% relative humidity (RH) comprising signals at the following two-theta values: 8.6±0.2, 17.2±0.2, 19.9±0.2, 20.4±0.2, 22.8±0.2, and 28.3±0.2.

In some embodiments, the Compound I Phosphate Salt Hydrate Form A is characterized by an X-ray powder diffractogram measured at 25±2° C. and 5% relative humidity (RH) comprising (a) a signal at the following two-theta values: 8.6±0.2, 19.9±0.2, and 28.3±0.2; and (b) a signal at one or more (e.g., two or more, three or more, four or more) two-theta values chosen from 17.2±0.2, 20.4±0.2, 21.1±0.2, 21.9±0.2, and 22.8±0.2. In some embodiments, the Compound I Phosphate Salt Hydrate Form A is characterized by an X-ray powder diffractogram measured at 25±2° C. and 5% relative humidity (RH) comprising a signal at one or more two-theta values (e.g., two or more, three or more, four or more, five or more, six or more, seven or more) chosen from 8.6±0.2, 17.2±0.2, 19.9±0.2, 20.4±0.2, 21.1±0.2, 21.9±0.2, 22.8±0.2, and 28.3±0.2. In some embodiments, the Compound I Phosphate Salt Hydrate Form A is characterized by an X-ray powder diffractogram measured at 25±2° C. and 5% relative humidity (RH) comprising signals at the following two-theta values: 8.6±0.2, 17.2±0.2, 19.9±0.2, 20.4±0.2, 21.1±0.2, 21.9±0.2, 22.8±0.2, and 28.3±0.2.

In some embodiments, the Compound I Phosphate Salt Hydrate Form A is characterized by an X-ray powder diffractogram measured at 25±2° C. and 5% relative humidity (RH) comprising (a) a signal at the following two-theta values: 8.6±0.2, 19.9±0.2, and 28.3±0.2; and (b) a signal at one or more (e.g., two or more, three or more, four or more, five or more, six or more) two-theta values chosen from 15.7±0.2, 17.2±0.2, 20.4±0.2, 21.1±0.2, 21.9±0.2, 22.8±0.2, and 27.0±0.2. In some embodiments, the Compound I Phosphate Salt Hydrate Form A is characterized by an X-ray powder diffractogram measured at 25±2° C. and 5% relative humidity (RH) comprising a signal at one or more two-theta values (e.g., two or more, three or more, four or more, five or more, six or more, seven or more, eight or more, nine or more) chosen from 8.6±0.2, 15.7±0.2, 17.2±0.2, 19.9±0.2, 20.4±0.2, 21.1±0.2, 21.9±0.2, 22.8±0.2, 27.0±0.2, and 28.3±0.2. In some embodiments, the Compound I Phosphate Salt Hydrate Form A is characterized by an X-ray powder diffractogram measured at 25±2° C. and 5% relative humidity (RH) comprising signals at the following two-theta values: 8.6±0.2, 15.7±0.2, 17.2 0.2, 19.9±0.2, 20.4±0.2, 21.1±0.2, 21.9±0.2, 22.8±0.2, 27.0±0.2, and 28.3±0.2.

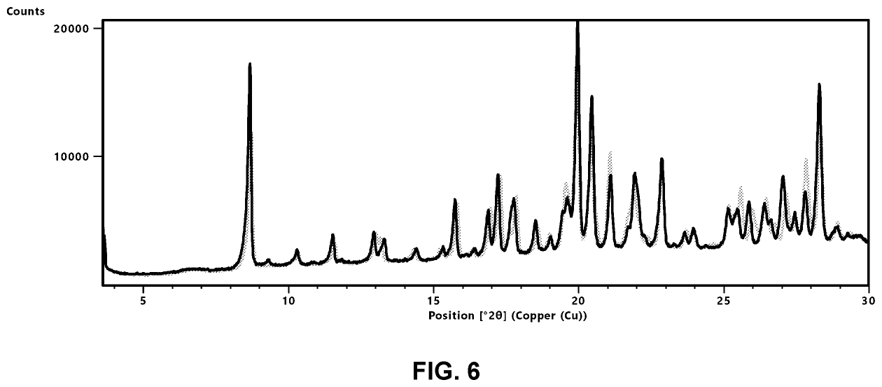

In some embodiments, the Compound I Phosphate Salt Hydrate Form A is characterized by an X-ray powder diffractogram measured at 25±2° C. and 5% relative humidity (RH) substantially similar to that in FIG. 6.

In some embodiments, the Compound I Phosphate Salt Hydrate Form A is characterized by an X-ray powder diffractogram measured at 25±2° C. and 40% relative humidity (RH) comprising (a) a signal at the following two-theta values: 8.6±0.2, 19.9±0.2, and 28.3±0.2; and (b) a signal at one or more (e.g., two or more) two-theta values chosen from 20.4±0.2, 21.0±0.2, and 22.8±0.2. In some embodiments, the Compound I Phosphate Salt Hydrate Form A is characterized by an X-ray powder diffractogram measured at 25±2° C. and 40% relative humidity (RH) comprising a signal at one or more two-theta values (e.g., two or more, three or more, four or more, five or more) chosen from 8.6±0.2, 19.9±0.2, 20.4±0.2, 21.0±0.2, 22.8±0.2, and 28.3±0.2. In some embodiments, the Compound I Phosphate Salt Hydrate Form A is characterized by an X-ray powder diffractogram measured at 25±2° C. and 40% relative humidity (RH) comprising signals at the following two-theta values: 8.6±0.2, 19.9±0.2, 20.4±0.2, 21.0±0.2, 22.8±0.2, and 28.3±0.2.

In some embodiments, the Compound I Phosphate Salt Hydrate Form A is characterized by an X-ray powder diffractogram measured at 25±2° C. and 40% relative humidity (RH) comprising (a) a signal at the following two-theta values: 8.6±0.2, 19.9±0.2, and 28.3±0.2; and (b) a signal at one or more (e.g., two or more, three or more, four or more) two-theta values chosen from 17.2±0.2, 20.4±0.2, 21.0±0.2, 22.8±0.2, and 27.8±0.2. In some embodiments, the Compound I Phosphate Salt Hydrate Form A is characterized by an X-ray powder diffractogram measured at 25±2° C. and 40% relative humidity (RH) comprising a signal at one or more two-theta values (e.g., two or more, three or more, four or more, five or more, six or more, seven or more) chosen from 8.6±0.2, 17.2±0.2, 19.9±0.2, 20.4±0.2, 21.0±0.2, 22.8±0.2, 27.8±0.2, and 28.3±0.2. In some embodiments, the Compound I Phosphate Salt Hydrate Form A is characterized by an X-ray powder diffractogram measured at 25±2° C. and 40% relative humidity (RH) comprising signals at the following two-theta values: 8.6±0.2, 17.2 0.2, 19.9±0.2, 20.4±0.2, 21.0±0.2, 22.8±0.2, 27.8±0.2, and 28.3±0.2.

In some embodiments, the Compound I Phosphate Salt Hydrate Form A is characterized by an X-ray powder diffractogram measured at 25±2° C. and 40% relative humidity (RH) comprising (a) a signal at the following two-theta values: 8.6±0.2, 19.9±0.2, and 28.3±0.2; and (b) a signal at one or more (e.g., two or more, three or more, four or more, five or more, six or more) two-theta values chosen from 17.2±0.2, 17.8±0.2, 20.4±0.2, 21.0±0.2, 22.8±0.2, 26.4±0.2, and 27.8±0.2. In some embodiments, the Compound I Phosphate Salt Hydrate Form A is characterized by an X-ray powder diffractogram measured at 25±2° C. and 40% relative humidity (RH) comprising a signal at one or more two-theta values (e.g., two or more, three or more, four or more, five or more, six or more, seven or more, eight or more, nine or more) chosen from 8.6±0.2, 17.2±0.2, 17.8±0.2, 19.9±0.2, 20.4±0.2, 21.0±0.2, 22.8±0.2, 26.4±0.2, 27.8±0.2, and 28.3±0.2. In some embodiments, the Compound I Phosphate Salt Hydrate Form A is characterized by an X-ray powder diffractogram measured at 2° C. and 40% relative humidity (RH) comprising signals at the following two-theta values: 8.6±0.2, 17.2 0.2, 17.8±0.2, 19.9±0.2, 20.4±0.2, 21.0±0.2, 22.8±0.2, 26.4±0.2, 27.8±0.2, and 28.3±0.2.

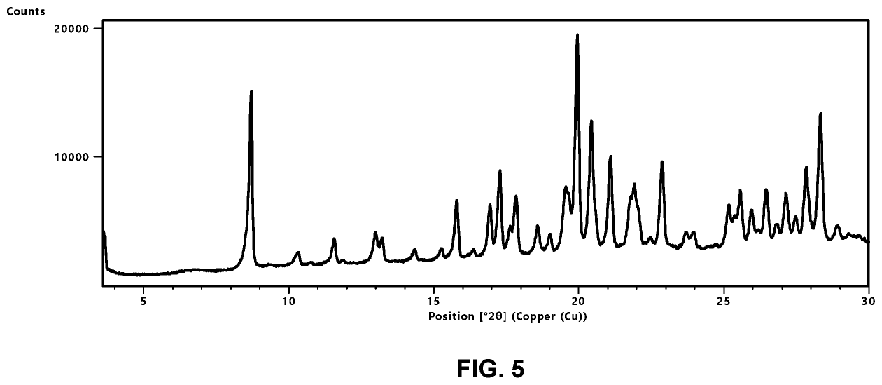

In some embodiments, the Compound I Phosphate Salt Hydrate Form A is characterized by an X-ray powder diffractogram measured at 25±2° C. and 40% relative humidity (RH) substantially similar to that in FIG. 5.

In some embodiments, the Compound I Phosphate Salt Hydrate Form A is characterized by an X-ray powder diffractogram measured at 25±2° C. and 90% relative humidity (RH) comprising (a) a signal at the following two-theta values: 8.6±0.2, 19.9±0.2, and 28.3±0.2; and (b) a signal at one or more (e.g., two or more) two-theta values chosen from 20.4±0.2, 21.0±0.2, and 27.8±0.2. In some embodiments, the Compound I Phosphate Salt Hydrate Form A is characterized by an X-ray powder diffractogram measured at 25±2° C. and 90% relative humidity (RH) comprising a signal at one or more two-theta values (e.g., two or more, three or more, four or more, five or more) chosen from 8.6±0.2, 19.9±0.2, 20.4±0.2, 21.0±0.2, 27.8±0.2, and 28.3±0.2. In some embodiments, the Compound I Phosphate Salt Hydrate Form A is characterized by an X-ray powder diffractogram measured at 25±2° C. and 90% relative humidity (RH) comprising signals at the following two-theta values: 8.6±0.2, 19.9±0.2, 20.4±0.2, 21.0±0.2, 27.8±0.2, and 28.3±0.2.

In some embodiments, the Compound I Phosphate Salt Hydrate Form A is characterized by an X-ray powder diffractogram measured at 25±2° C. and 90% relative humidity (RH) comprising (a) a signal at the following two-theta values: 8.6±0.2, 19.9±0.2, and 28.3±0.2; and (b) a signal at one or more (e.g., two or more, three or more, four or more) two-theta values chosen from 17.2±0.2, 20.4±0.2, 21.0±0.2, 22.8±0.2, and 27.8±0.2. In some embodiments, the Compound I Phosphate Salt Hydrate Form A is characterized by an X-ray powder diffractogram measured at 25±2° C. and 90% relative humidity (RH) comprising a signal at one or more two-theta values (e.g., two or more, three or more, four or more, five or more, six or more, seven or more) chosen from 8.6±0.2, 17.2±0.2, 19.9±0.2, 20.4±0.2, 21.0±0.2, 22.8±0.2, 27.8±0.2, and 28.3±0.2. In some embodiments, the Compound I Phosphate Salt Hydrate Form A is characterized by an X-ray powder diffractogram measured at 25±2° C. and 90% relative humidity (RH) comprising signals at the following two-theta values: 8.6±0.2, 17.2 0.2, 19.9±0.2, 20.4±0.2, 21.0±0.2, 22.8±0.2, 27.8±0.2, and 28.3±0.2.

In some embodiments, the Compound I Phosphate Salt Hydrate Form A is characterized by an X-ray powder diffractogram measured at 25±2° C. and 90% relative humidity (RH) comprising (a) a signal at the following two-theta values: 8.6±0.2, 19.9±0.2, and 28.3±0.2; and (b) a signal at one or more (e.g., two or more, three or more, four or more, five or more, six or more) two-theta values chosen from 17.2±0.2, 19.5±0.2, 20.4±0.2, 21.0±0.2, 22.8±0.2, 25.5±0.2, and 27.8±0.2. In some embodiments, the Compound I Phosphate Salt Hydrate Form A is characterized by an X-ray powder diffractogram measured at 25±2° C. and 90% relative humidity (RH) comprising a signal at one or more two-theta values (e.g., two or more, three or more, four or more, five or more, six or more, seven or more, eight or more, nine or more) chosen from 8.6±0.2, 17.2±0.2, 19.5±0.2, 19.9±0.2, 20.4±0.2, 21.0±0.2, 22.8±0.2, 25.5±0.2, 27.8±0.2, and 28.3±0.2. In some embodiments, the Compound I Phosphate Salt Hydrate Form A is characterized by an X-ray powder diffractogram measured at 2° C. and 90% relative humidity (RH) comprising signals at the following two-theta values: 8.6±0.2, 17.2 0.2, 19.5±0.2, 19.9±0.2, 20.4±0.2, 21.0±0.2, 22.8±0.2, 25.5±0.2, 27.8±0.2, and 28.3±0.2.

In some embodiments, the Compound I Phosphate Salt Hydrate Form A is characterized by an X-ray powder diffractogram measured at 25±2° C. and 90% relative humidity (RH) substantially similar to that in FIG. 6.

In some embodiments, the Compound I Phosphate Salt Hydrate Form A is characterized by a ¹³C NMR spectrum measured at 43% relative humidity (RH) comprising one or more signals chosen from 62.1±0.2 ppm, 62.7±0.2 ppm, 128.6±0.2 ppm, 139.3±0.2 ppm, and 141.7±0.2 ppm.

In some embodiments, the Compound I Phosphate Salt Hydrate Form A is characterized by a ¹³C NMR spectrum measured at 43% relative humidity (RH) comprising one or more (e.g., two or more, three or more, four or more) signals chosen from 16.0±0.2 ppm, 38.4±0.2 ppm, 128.6±0.2 ppm, 139.3±0.2 ppm, and 141.7±0.2 ppm.

In some embodiments, the Compound I Phosphate Salt Hydrate Form A is characterized by a ¹³C NMR spectrum measured at 43% relative humidity (RH) comprising one or more signals chosen from 16.0±0.2 ppm, 38.4±0.2 ppm, 47.3±0.2 ppm, 62.1±0.2 ppm, 62.7±0.2 ppm, 73.2±0.2 ppm, 73.6±0.2 ppm, 128.6±0.2 ppm, 139.3±0.2 ppm, and 141.7±0.2 ppm.

In some embodiments, the Compound I Phosphate Salt Hydrate Form A is characterized by a ¹³C NMR spectrum measured at 43% relative humidity (RH) comprising one or more (e.g., two or more, three or more, four or more, five or more, six or more, seven or more, eight or more, nine or more) signals chosen from 16.0±0.2 ppm, 36.7±0.2 ppm, 38.4±0.2 ppm, 126.6±0.2 ppm, 128.6±0.2 ppm, 129.4±0.2 ppm, 139.3±0.2 ppm, 141.7±0.2 ppm, 144.0±0.2 ppm, and 145.8±0.2 ppm.

In some embodiments, the Compound I Phosphate Salt Hydrate Form A is characterized by a ¹³C NMR spectrum measured at 43% relative humidity (RH) comprising signals at 62.1±0.2 ppm, 62.7±0.2 ppm, 128.6±0.2 ppm, 139.3±0.2 ppm, and 141.7±0.2 ppm.

In some embodiments, the Compound I Phosphate Salt Hydrate Form A is characterized by a ¹³C NMR spectrum measured at 43% relative humidity (RH) comprising signals at 16.0±0.2 ppm, 38.4±0.2 ppm, 128.6±0.2 ppm, 139.3±0.2 ppm, and 141.7±0.2 ppm.

In some embodiments, the Compound I Phosphate Salt Hydrate Form A is characterized by a ¹³C NMR spectrum measured at 43% relative humidity (RH) comprising signals at 16.0±0.2 ppm, 38.4±0.2 ppm, 47.3±0.2 ppm, 62.1±0.2 ppm, 62.7±0.2 ppm, 73.2±0.2 ppm, 73.6±0.2 ppm, 128.6±0.2 ppm, 139.3±0.2 ppm, and 141.7±0.2 ppm.

In some embodiments, the Compound I Phosphate Salt Hydrate Form A is characterized by a ¹³C NMR spectrum measured at 43% relative humidity (RH) comprising signals at 16.0±0.2 ppm, 36.7±0.2 ppm, 38.4±0.2 ppm, 126.6±0.2 ppm, 128.6±0.2 ppm, 129.4±0.2 ppm, 139.3±0.2 ppm, 141.7±0.2 ppm, 144.0±0.2 ppm, and 145.8±0.2 ppm.

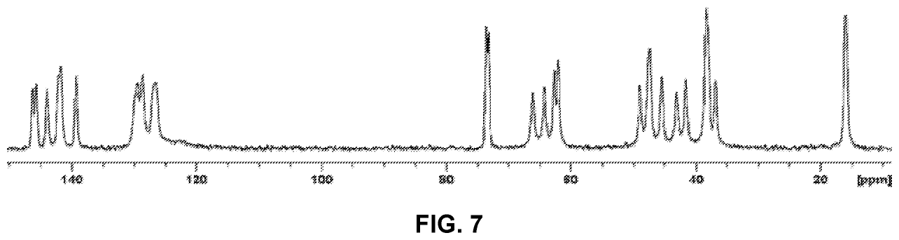

In some embodiments, the Compound I Phosphate Salt Hydrate Form A is characterized by a ¹³C NMR spectrum measured at 43% relative humidity (RH) substantially similar to that in FIG. 7.

In some embodiments, the Compound I Phosphate Salt Hydrate Form A is characterized by a ¹⁹F NMR spectrum measured at 43% relative humidity (RH) comprising a signal at one or more ppm values chosen from −57.4±0.2 ppm and −53.8±0.2 ppm.

In some embodiments, the Compound I Phosphate Salt Hydrate Form A is characterized by a ¹⁹F NMR spectrum measured at 43% relative humidity (RH) comprising signals at −57.4±0.2 ppm and −53.8±0.2 ppm.

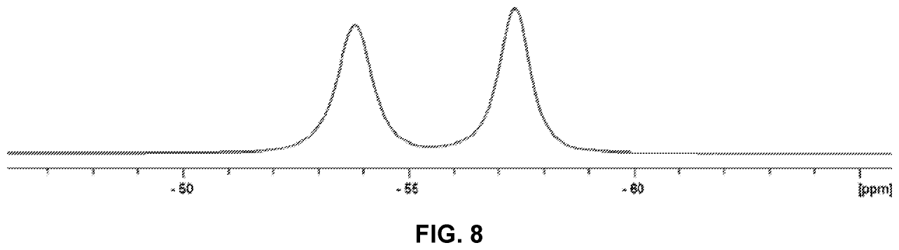

In some embodiments, the Compound I Phosphate Salt Hydrate Form A is characterized by a ¹⁹F NMR spectrum measured at 43% relative humidity (RH) substantially similar to that in FIG. 8.

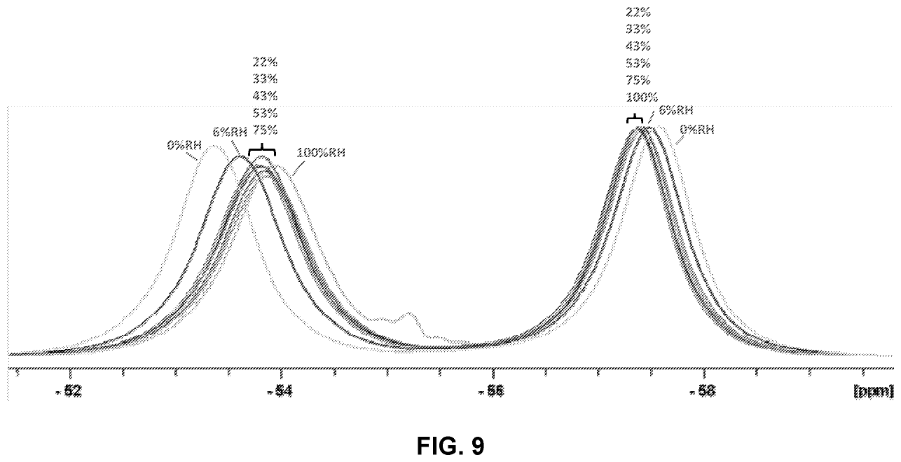

In some embodiments, the Compound I Phosphate Salt Hydrate Form A is characterized by a ¹⁹F NMR spectrum measured at 0% relative humidity (RH), 6% RH, 22%, 33% RH, 43% RH, 53% RH, 75% RH, or 100% RH substantially similar to that in FIG. 9.

In some embodiments, the Compound I Phosphate Salt Hydrate Form A is characterized by a ³¹P NMR spectrum measured at 43% relative humidity (RH) comprising a signal at one or more ppm values chosen from 2.6±0.2 ppm and 4.2±0.2 ppm.

In some embodiments, the Compound I Phosphate Salt Hydrate Form A is characterized by a ³¹P NMR spectrum measured at 43% relative humidity (RH) comprising signals at 2.6±0.2 ppm and 4.2±0.2 ppm.

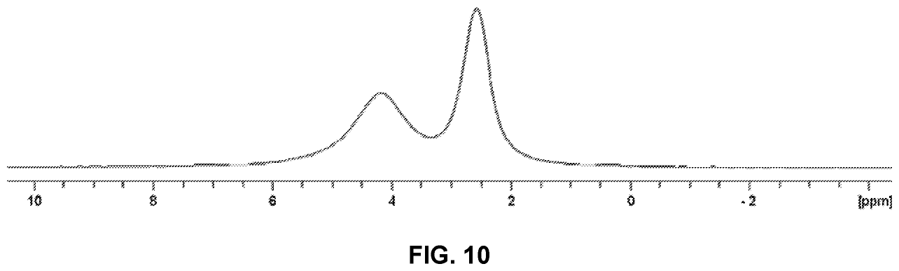

In some embodiments, the Compound I Phosphate Salt Hydrate Form A is characterized by a ³¹P NMR spectrum measured at 43% relative humidity (RH) substantially similar to that in FIG. 10.

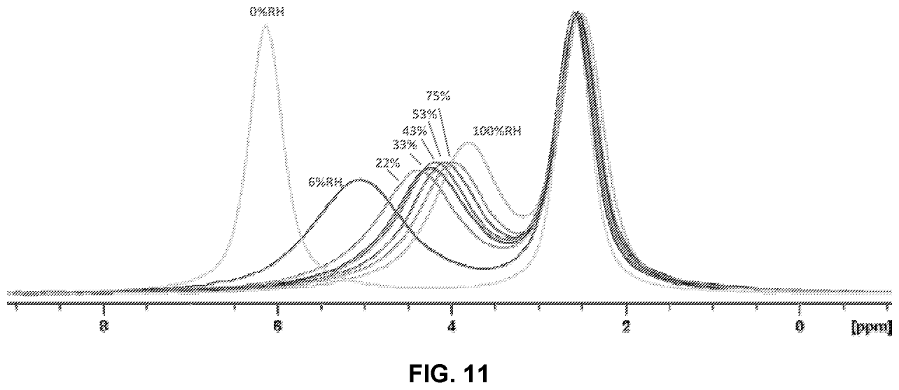

In some embodiments, the Compound I Phosphate Salt Hydrate Form A is characterized by a ³¹P NMR spectrum measured at 0% relative humidity (RH), 6% RH, 22%, 33% RH, 43% RH, 53% RH, 75% RH, or 100% RH substantially similar to that in FIG. 11.

In some embodiments, the Compound I Phosphate Salt Hydrate Form A is characterized by a TGA thermogram showing 0.5% weight loss from ambient temperature to 150° C.

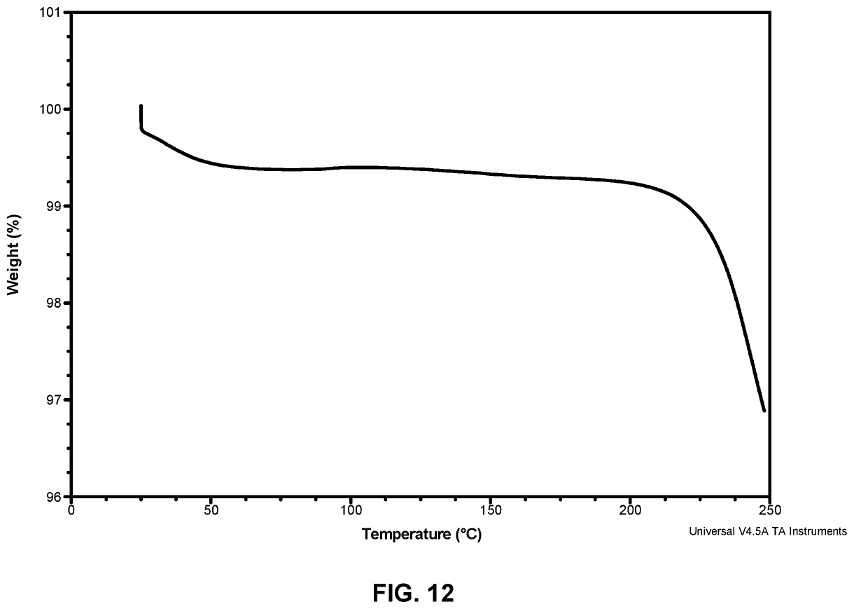

In some embodiments, the Compound I Phosphate Salt Hydrate Form A is characterized by a TGA thermogram substantially similar to that in FIG. 12.

In some embodiments, the Compound I Phosphate Salt Hydrate Form A is characterized by a DSC curve comprising two endotherm peaks at about 226° C. and about 251° C.

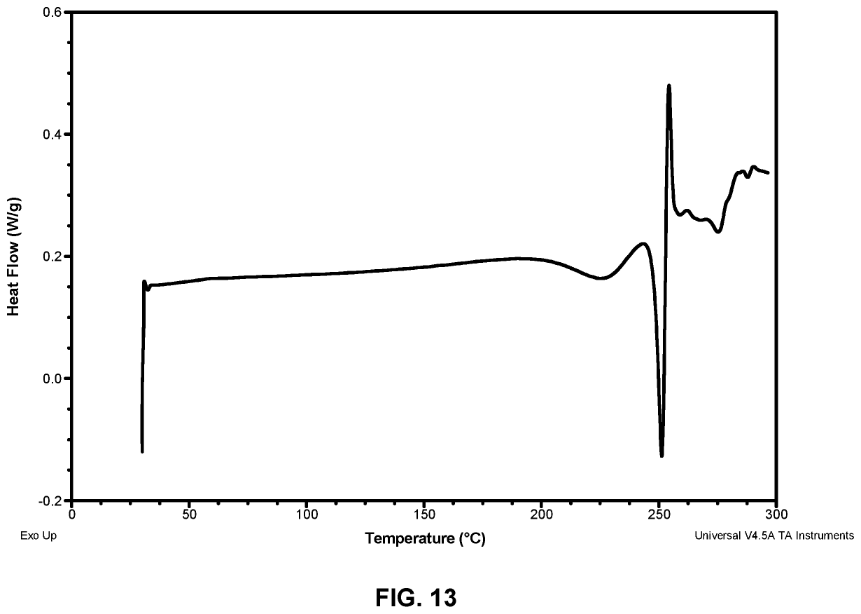

In some embodiments, the Compound I Phosphate Salt Hydrate Form A is characterized by a DSC curve substantially similar to that in FIG. 13.

In some embodiments, the Compound I Phosphate Salt Hydrate Form A is characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 8.9 ± 0.1 Å | α | 90° |
| b | 10.5 ± 0.1 Å | β | 90° |
| c | 45.0 ± 0.1 Å | γ | 90°. |

In some embodiments, the Compound I Phosphate Salt Hydrate Form A is characterized by an orthorhombic crystal system, a P2₁2₁2₁ space group, and unit cell dimensions measured at 100 K on a Bruker diffractometer equipped Cu Kα radiation (λ=1.54178 Å) after drying at 300 K under dry nitrogen for 1 hour of

| | | | |
|---|---|---|---|
| a | 8.8 ± 0.1 Å | α | 90° |
| b | 10.5 ± 0.1 Å | β | 90° |
| c | 45.2 ± 0.1 Å | γ | 90°. |

Some embodiments of the disclosure provide a method of preparing Compound I Phosphate Salt Hydrate Form A comprising drying Compound I Phosphate Salt Methanol Solvate at about 50° C.

In some embodiments, the method comprises drying Compound I Phosphate Salt Methanol Solvate at about 50° C. for about 21 hours with nitrogen purge.

Some embodiments of the disclosure provide a method of preparing Compound I Phosphate Salt Hydrate Form A comprising:

charging Compound I free form Monohydrate and MEK to a reactor;

agitating the reactor (e.g., at about 20° C.);

adding water to the reactor and further agitating;

seeding the reactor with Compound I Phosphate Salt Hydrate Form A;

slowly adding an 0.5 M phosphoric acid in MEK/water solution to the reactor; and

agitating the reactor at about 20° C.

In some embodiments, the method further comprises isolating a wet cake, washing the wet cake with MEK, and drying the wet cake under vacuum.

Some embodiments of the disclosure provide a method of preparing Compound I Phosphate Salt Hydrate Form A comprising:

charging Compound I Monohydrate and MEK to a reactor;

agitating the reactor;

adding water to the reactor and further agitating;

slowly adding an 0.5 M phosphoric acid in MEK/water solution to the reactor; and

agitating the reactor at about 20° C.

In some embodiments, the method further comprises isolating a wet cake, washing the wet cake with MEK, and drying the wet cake under vacuum.

### Compound I Free Form Monohydrate

Some embodiments of the disclosure provide a monohydrate form of Compound I (Compound I free form Monohydrate). In some embodiments, the Compound I free form Monohydrate is substantially pure.

In some embodiments, the Compound I free form Monohydrate is characterized by an X-ray powder diffractogram comprising a signal at 8.7, 12.8, 16.7, and/or 21.7±0.2 two-theta.

In some embodiments, the Compound I free form Monohydrate is characterized by an X-ray powder diffractogram comprising a signal at one or more (e.g., two or more, three or more) two-theta values chosen from 8.7±0.2, 12.8±0.2, 16.7±0.2, and 21.7±0.2. In some embodiments, the Compound I free form Monohydrate is characterized by an X-ray powder diffractogram comprising a signal at two or more two-theta values chosen from 8.7±0.2, 12.8±0.2, 16.7±0.2, and 21.7±0.2. In some embodiments, the Compound I free form Monohydrate is characterized by an X-ray powder diffractogram comprising a signal at three or more two-theta values chosen from 8.7±0.2, 12.8±0.2, 16.7±0.2, and 21.7±0.2.

In some embodiments, the Compound I free form Monohydrate is characterized by an X-ray powder diffractogram comprising a signal at the following two-theta values: 8.7±0.2, 12.8±0.2, 16.7±0.2, and 21.7±0.2. In some embodiments, the Compound I free form Monohydrate is characterized by an X-ray powder diffractogram comprising (a) a signal at the following two-theta values: 8.7±0.2, 12.8±0.2, 16.7±0.2, and 21.7±0.2; and (b) a signal at one or more (e.g., two or more) two-theta values chosen from 13.8±0.2, 19.8±0.2, and 25.8±0.2. In some embodiments, the Compound I free form Monohydrate is characterized by an X-ray powder diffractogram comprising a signal at one or more two-theta values (e.g., two or more, three or more, four or more, five or more, six or more) chosen from 8.7±0.2, 12.8±0.2, 13.8±0.2, 16.7±0.2, 19.8±0.2, 21.7±0.2, and 25.8±0.2. In some embodiments, the Compound I free form Monohydrate is characterized by an X-ray powder diffractogram comprising signals at the following two-theta values: 8.7±0.2, 12.8±0.2, 13.8±0.2, 16.7±0.2, 19.8±0.2, 21.7±0.2, and 25.8±0.2.

In some embodiments, the Compound I free form Monohydrate is characterized by an X-ray powder diffractogram comprising (a) a signal at the following two-theta values: 8.7±0.2, 12.8±0.2, 16.7±0.2, and 21.7±0.2; and (b) a signal at one or more (e.g., two or more, three or more, four or more) two-theta values chosen from 13.8±0.2, 15.5±0.2, 19.8±0.2, 24.3±0.2, and 25.8±0.2. In some embodiments, the Compound I free form Monohydrate is characterized by an X-ray powder diffractogram comprising a signal at one or more two-theta values (e.g., two or more, three or more, four or more, five or more, six or more, seven or more, eight or more) chosen from 8.7±0.2, 12.8±0.2, 13.8±0.2, 15.5±0.2, 16.7±0.2, 19.8±0.2, 21.7±0.2, 24.3±0.2, and 25.8±0.2. In some embodiments, the Compound I free form Monohydrate is characterized by an X-ray powder diffractogram comprising signals at the following two-theta values: 8.7±0.2, 12.8±0.2, 13.8±0.2, 15.5±0.2, 16.7±0.2, 19.8±0.2, 21.7±0.2, 24.3±0.2, and 25.8±0.2.

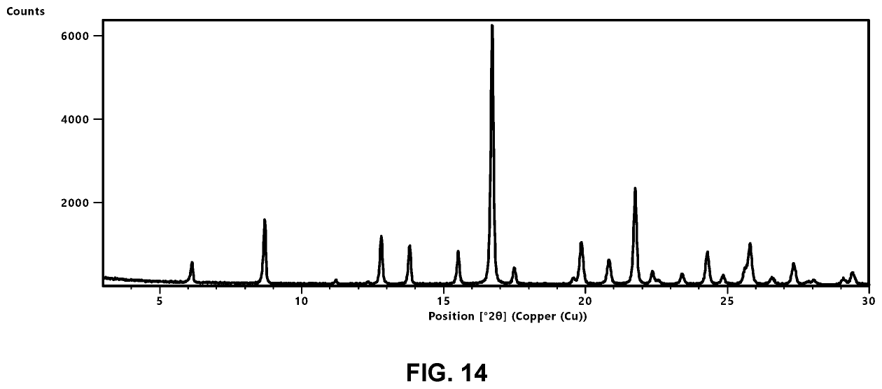

In some embodiments, the Compound I free form Monohydrate is characterized by an X-ray powder diffractogram substantially similar to that in FIG. 14.

In some embodiments, the Compound I free form Monohydrate is characterized by a ¹³C NMR spectrum measured at 43% relative humidity (RH) comprising one or more (e.g., two or more, three or more, four or more) signals chosen from 24.9±0.2 ppm, 49.8±0.2 ppm, 74.4±0.2 ppm, 135.3±0.2 ppm, and 149.6±0.2 ppm.

In some embodiments, the Compound I free form Monohydrate is characterized by a ¹³C NMR spectrum measured at 43% relative humidity (RH) comprising one or more (e.g., two or more, three or more, four or more) signals chosen from 24.9±0.2 ppm, 35.1±0.2 ppm, 39.3±0.2 ppm, 135.3±0.2 ppm, and 149.6±0.2 ppm.

In some embodiments, the Compound I free form Monohydrate is characterized by a ¹³C NMR spectrum measured at 43% relative humidity (RH) comprising one or more (e.g., two or more, three or more, four or more, five or more, six or more, seven or more, eight or more, nine or more) signals chosen from 24.9±0.2 ppm, 35.1±0.2 ppm, 39.3±0.2 ppm, 47.0±0.2 ppm, 49.8±0.2 ppm, 61.6±0.2 ppm, 68.1±0.2 ppm, 74.4±0.2 ppm, 135.3±0.2 ppm, and 149.6±0.2 ppm.

In some embodiments, the Compound I free form Monohydrate is characterized by a ¹³C NMR spectrum measured at 43% relative humidity (RH) comprising one or more (e.g., two or more, three or more, four or more, five or more, six or more, seven or more, eight or more) signals chosen from 24.9±0.2 ppm, 35.1±0.2 ppm, 39.3±0.2 ppm, 126.2±0.2 ppm, 127.7±0.2 ppm, 129.6±0.2 ppm, 135.3±0.2 ppm, 149.4±0.2 ppm, and 149.6±0.2 ppm.

In some embodiments, the Compound I free form Monohydrate is characterized by a ¹³C NMR spectrum measured at 43% relative humidity (RH) comprising signals at 24.9±0.2 ppm, 49.8±0.2 ppm, 74.4±0.2 ppm, 135.3±0.2 ppm, and 149.6±0.2 ppm.

In some embodiments, the Compound I free form Monohydrate is characterized by a ¹³C NMR spectrum measured at 43% relative humidity (RH) comprising signals at 24.9±0.2 ppm, 35.1±0.2 ppm, 39.3±0.2 ppm, 135.3±0.2 ppm, and 149.6±0.2 ppm.

In some embodiments, the Compound I free form Monohydrate is characterized by a ¹³C NMR spectrum measured at 43% relative humidity (RH) comprising signals at 24.9±0.2 ppm, 35.1±0.2 ppm, 39.3±0.2 ppm, 47.0±0.2 ppm, 49.8±0.2 ppm, 61.6±0.2 ppm, 68.1±0.2 ppm, 74.4±0.2 ppm, 135.3±0.2 ppm, and 149.6±0.2 ppm.

In some embodiments, the Compound I free form Monohydrate is characterized by a ¹³C NMR spectrum measured at 43% relative humidity (RH) comprising signals at 24.9±0.2 ppm, 35.1±0.2 ppm, 39.3±0.2 ppm, 126.2±0.2 ppm, 127.7±0.2 ppm, 129.6±0.2 ppm, 135.3±0.2 ppm, 149.4±0.2 ppm, and 149.6±0.2 ppm.

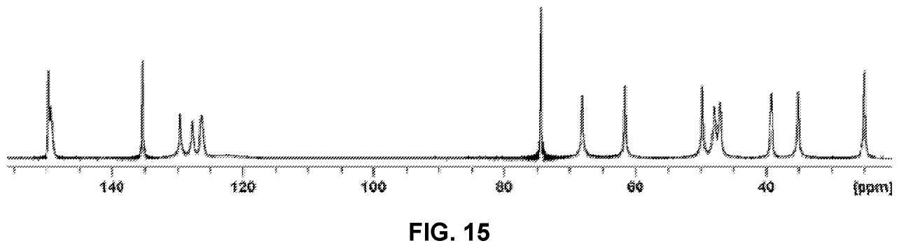

In some embodiments, the Compound I free form Monohydrate is characterized by a ¹³C NMR spectrum measured at 43% relative humidity (RH) substantially similar to that in FIG. 15.

In some embodiments, the Compound I free form Monohydrate is characterized by a ¹³C NMR spectrum measured after dehydration (80° C. in rotor overnight (2×), 80° C. weekend incubation with P205) comprising one or more (e.g., two or more, three or more, four or more) signals chosen from 25.6±0.2 ppm, 50.7±0.2 ppm, 74.7±0.2 ppm, 135.3±0.2 ppm, and 150±0.2 ppm.

In some embodiments, the Compound I free form Monohydrate is characterized by a ¹³C NMR spectrum measured after dehydration (80° C. in rotor overnight (2×), 80° C. weekend incubation with P205) comprising one or more (e.g., two or more, three or more, four or more) signals chosen from 25.6±0.2 ppm, 35.8±0.2 ppm, 36.8±0.2 ppm, 135.3±0.2 ppm, and 150±0.2 ppm.

In some embodiments, the Compound I free form Monohydrate is characterized by a ¹³C NMR spectrum measured after dehydration (80° C. in rotor overnight (2×), 80° C. weekend incubation with P205) comprising one or more (e.g., two or more, three or more, four or more, five or more, six or more, seven or more, eight or more, nine or more) signals chosen from 25.6±0.2 ppm, 35.8±0.2 ppm, 36.8±0.2 ppm, 47.2±0.2 ppm, 48.3±0.2 ppm, 50.7±0.2 ppm, 61.5±0.2 ppm, 74.7±0.2 ppm, 135.3±0.2 ppm, and 150±0.2 ppm.

In some embodiments, the Compound I free form Monohydrate is characterized by a ¹³C NMR spectrum measured after dehydration (80° C. in rotor overnight (2×), 80° C. weekend incubation with P205) comprising one or more (e.g., two or more, three or more, four or more, five or more, six or more, seven or more, eight or more) signals chosen from 25.6±0.2 ppm, 35.8±0.2 ppm, 36.8±0.2 ppm, 126.6±0.2 ppm, 127.2±0.2 ppm, 129.6±0.2 ppm, 135.3±0.2 ppm, 150±0.2 ppm, and 150.9±0.2 ppm.

In some embodiments, the Compound I free form Monohydrate is characterized by a ¹³C NMR spectrum measured after dehydration (80° C. in rotor overnight (2×), 80° C. weekend incubation with P205) comprising signals at 25.6±0.2 ppm, 50.7±0.2 ppm, 74.7±0.2 ppm, 135.3±0.2 ppm, and 150±0.2 ppm.

In some embodiments, the Compound I free form Monohydrate is characterized by a ¹³C NMR spectrum measured after dehydration (80° C. in rotor overnight (2×), 80° C. weekend incubation with P205) comprising signals at 25.6±0.2 ppm, 35.8±0.2 ppm, 36.8±0.2 ppm, 135.3±0.2 ppm, and 150±0.2 ppm.

In some embodiments, the Compound I free form Monohydrate is characterized by a ¹³C NMR spectrum measured after dehydration (80° C. in rotor overnight (2×), 80° C. weekend incubation with P205) comprising signals at 25.6±0.2 ppm, 35.8±0.2 ppm, 36.8±0.2 ppm, 47.2±0.2 ppm, 48.3±0.2 ppm, 50.7±0.2 ppm, 61.5±0.2 ppm, 74.7±0.2 ppm, 135.3±0.2 ppm, and 150±0.2 ppm.

In some embodiments, the Compound I free form Monohydrate is characterized by a ¹³C NMR spectrum measured after dehydration (80° C. in rotor overnight (2×), 80° C. weekend incubation with P205) comprising signals at 25.6±0.2 ppm, 35.8±0.2 ppm, 36.8±0.2 ppm, 126.6±0.2 ppm, 127.2±0.2 ppm, 129.6±0.2 ppm, 135.3±0.2 ppm, 150±0.2 ppm, and 150.9±0.2 ppm.

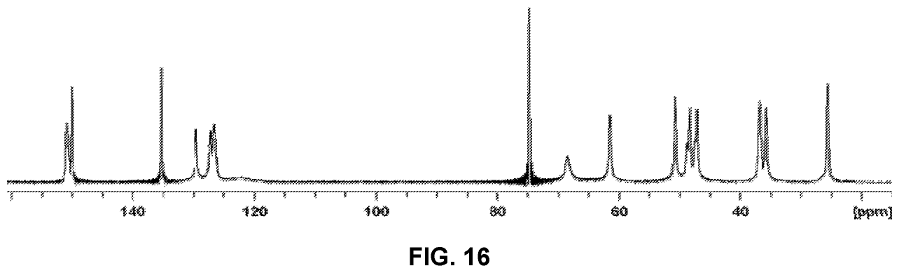

In some embodiments, the Compound I free form Monohydrate is characterized by a ¹³C NMR spectrum measured after dehydration (80° C. in rotor overnight (2×), 80° C. weekend incubation with P205) substantially similar to that in FIG. 16.

In some embodiments, the Compound I free form Monohydrate is characterized by a ¹⁹F NMR spectrum measured at 43% relative humidity (RH) comprising a signal at −55.8±0.2 ppm.

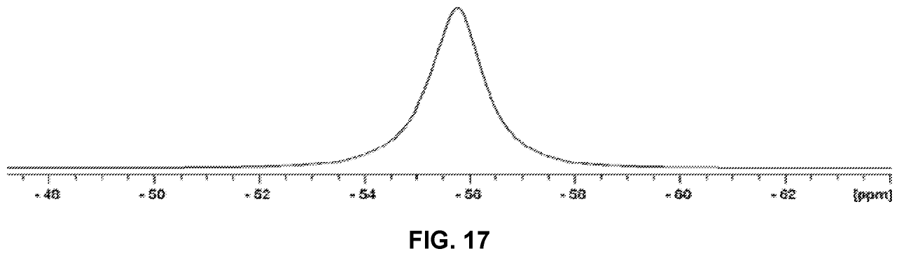

In some embodiments, the Compound I free form Monohydrate is characterized by a ¹⁹F NMR spectrum measured at 43% relative humidity (RH) substantially similar to that in FIG. 17.

In some embodiments, the Compound I free form Monohydrate is characterized by a ¹⁹F NMR spectrum measured after dehydration (80° C. in rotor overnight (2×), 80° C. weekend incubation with P205) comprising a signal at −55.5±0.2 ppm.

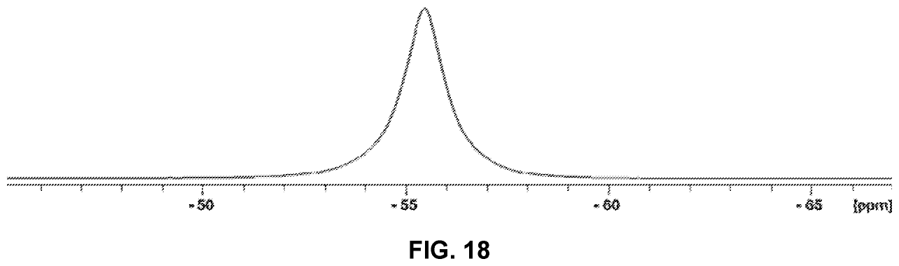

In some embodiments, the Compound I free form Monohydrate is characterized by a ¹⁹F NMR spectrum measured after dehydration (80° C. in rotor overnight (2×), 80° C. weekend incubation with P205) substantially similar to that in FIG. 18.

In some embodiments, the Compound I free form Monohydrate is characterized by a TGA thermogram showing about 3% to about 4% weight loss from ambient temperature to 100° C.

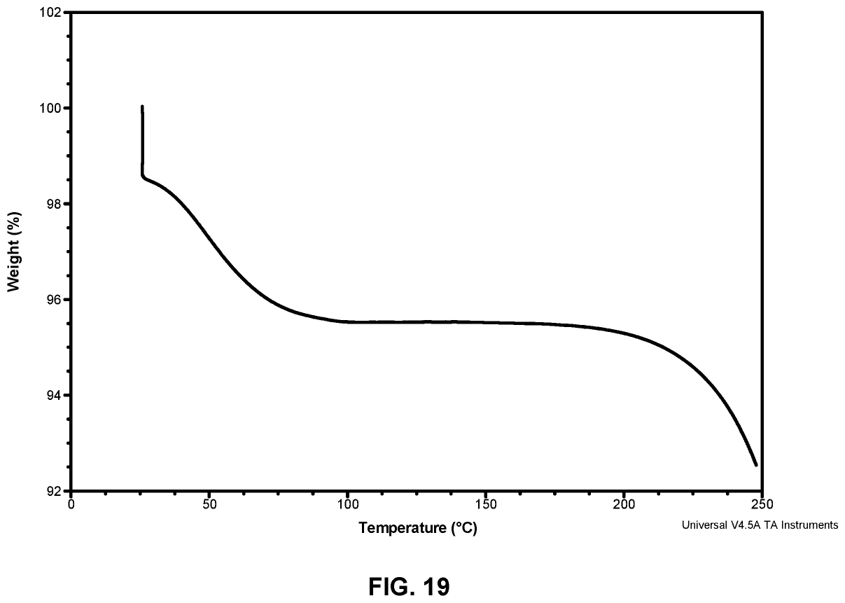

In some embodiments, the Compound I free form Monohydrate is characterized by a TGA thermogram substantially similar to that in FIG. 19.

In some embodiments, the Compound I free form Monohydrate is characterized by a DSC curve comprising endotherm peaks at about 61° C., about 94° C., and about 111° C.

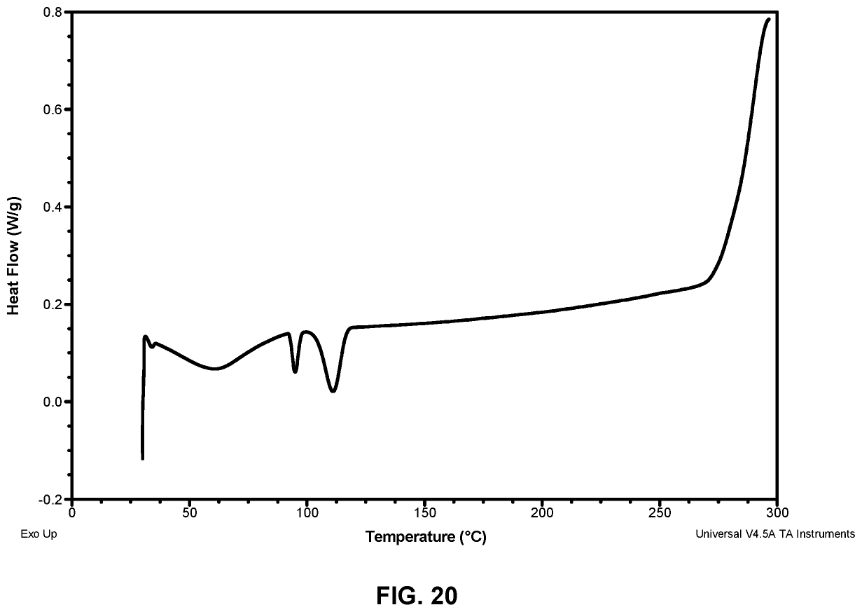

In some embodiments, the Compound I free form Monohydrate is characterized by a DSC curve substantially similar to that in FIG. 20.

In some embodiments, the Compound I free form Monohydrate is characterized by a tetragonal crystal system, a P4₃ space group, and unit cell dimensions measured at 100 K on a Bruker diffractometer equipped Cu Kα radiation (λ=1.54178 Å) of:

| | | | |
|---|---|---|---|
| a | 14.2 ± 0.1 Å | α | 90° |
| b | 14.2 ± 0.1 Å | β | 90° |
| c | 9.3 ± 0.1 Å | γ | 90°. |

In some embodiments, the Compound I free form Monohydrate is characterized by a tetragonal crystal system, a P43 space group, and unit cell dimensions measured at 100 K on a Bruker diffractometer equipped Cu Kα radiation (λ=1.54178 Å) after drying at 325 K under dry nitrogen for 1 hour of:

| | | | |
|---|---|---|---|
| a | 14.3 ± 0.1 Å | α | 90° |
| b | 14.3 ± 0.1 Å | β | 90° |
| c | 9.2 ± 0.1 Å | γ | 90°. |

Some embodiments of the disclosure provide a method of preparing Compound I free form Monohydrate comprising:

adding amorphous Compound I to saline to create a solution;

incubating the solution at ambient temperature;

filtering the solution to obtain a solid material; and

drying the solid material.

In some embodiments, incubating the solution at ambient temperature comprises incubating the solution at ambient temperature overnight.

In some embodiments, drying the solid material comprises drying the solid material in a vacuum oven at about 45° C. overnight.

### Compound I Phosphate Salt Methanol Solvate

Some embodiments of the disclosure provide a phosphate salt methanol solvate of Compound I (Compound I Phosphate Salt Methanol Solvate). In some embodiments, Compound I Phosphate Salt Methanol Solvate is substantially pure.

In some embodiments, the Compound I Phosphate Salt Methanol Solvate is characterized by an X-ray powder diffractogram comprising a signal at 12.7, 14.8, and/or 20.7±0.2 two-theta.

In some embodiments, the Compound I Phosphate Salt Methanol Solvate is characterized by an X-ray powder diffractogram comprising a signal at one or more two-theta values chosen from 12.7±0.2, 14.8±0.2, and 20.7±0.2. In some embodiments, the Compound I Phosphate Salt Methanol Solvate is characterized by an X-ray powder diffractogram comprising a signal at two or more two-theta values chosen from 12.7±0.2, 14.8±0.2, and 20.7±0.2.

In some embodiments, the Compound I Phosphate Salt Methanol Solvate is characterized by an X-ray powder diffractogram comprising a signal at the following two-theta values: 12.7±0.2, 14.8±0.2, and 20.7±0.2. In some embodiments, the Compound I Phosphate Salt Methanol Solvate is characterized by an X-ray powder diffractogram comprising (a) a signal at the following two-theta values: 12.7±0.2, 14.8±0.2, and 20.7±0.2; and (b) a signal at one or more two-theta values (e.g., two or more) chosen from 8.5±0.2, 15.8±0.2, and 19.5±0.2. In some embodiments, the Compound I Phosphate Salt Methanol Solvate is characterized by an X-ray powder diffractogram comprising a signal at one or more two-theta values (e.g., two or more, three or more, four or more, five or more) chosen from 8.5±0.2, 12.7±0.2, 14.8±0.2, 15.8±0.2, 19.5±0.2, and 20.7±0.2. In some embodiments, the Compound I Phosphate Salt Methanol Solvate is characterized by an X-ray powder diffractogram comprising signals at the following two-theta values: 8.5±0.2, 12.7±0.2, 14.8±0.2, 15.8±0.2, 19.5±0.2, and 20.7±0.2.

In some embodiments, the Compound I Phosphate Salt Methanol Solvate is characterized by an X-ray powder diffractogram comprising (a) a signal at the following two-theta values: 12.7±0.2, 14.8±0.2, and 20.7±0.2; and (b) a signal at one or more two-theta values (e.g., two or more, three or more, four or more) chosen from 8.5±0.2, 13.9±0.2, 15.8±0.2, 18.7±0.2, and 19.5±0.2. In some embodiments, the Compound I Phosphate Salt Methanol Solvate is characterized by an X-ray powder diffractogram comprising a signal at one or more two-theta values (e.g., two or more, three or more, four or more, five or more, six or more, seven or more) chosen from 8.5±0.2, 12.7±0.2, 13.9±0.2, 14.8±0.2, 15.8±0.2, 18.7±0.2, 19.5±0.2, and 20.7±0.2. In some embodiments, the Compound I Phosphate Salt Methanol Solvate is characterized by an X-ray powder diffractogram comprising signals at the following two-theta values: 8.5±0.2, 12.7±0.2, 13.9±0.2, 14.8±0.2, 15.8±0.2, 18.7±0.2, 19.5±0.2, and 20.7±0.2.

In some embodiments, the Compound I Phosphate Salt Methanol Solvate is characterized by an X-ray powder diffractogram comprising (a) a signal at the following two-theta values: 12.7±0.2, 14.8±0.2, and 20.7±0.2; and (b) a signal at one or more two-theta values (e.g., two or more, three or more, four or more, five or more, six or more) chosen from 8.5±0.2, 10.2 0.2, 13.9±0.2, 15.8±0.2, 18.7±0.2, 19.5±0.2, and 22.5±0.2. In some embodiments, the Compound I Phosphate Salt Methanol Solvate is characterized by an X-ray powder diffractogram comprising a signal at one or more two-theta values (e.g., two or more, three or more, four or more, five or more, six or more, seven or more, eight or more, nine or more) chosen from 8.5±0.2, 10.2±0.2, 12.7±0.2, 13.9±0.2, 14.8±0.2, 15.8±0.2, 18.7±0.2, 19.5±0.2, 20.7±0.2, and 22.5±0.2. In some embodiments, the Compound I Phosphate Salt Methanol Solvate is characterized by an X-ray powder diffractogram comprising signals at the following two-theta values: 8.5±0.2, 10.2±0.2, 12.7±0.2, 13.9±0.2, 14.8±0.2, 15.8±0.2, 18.7±0.2, 19.5±0.2, 20.7±0.2, and 22.5±0.2.

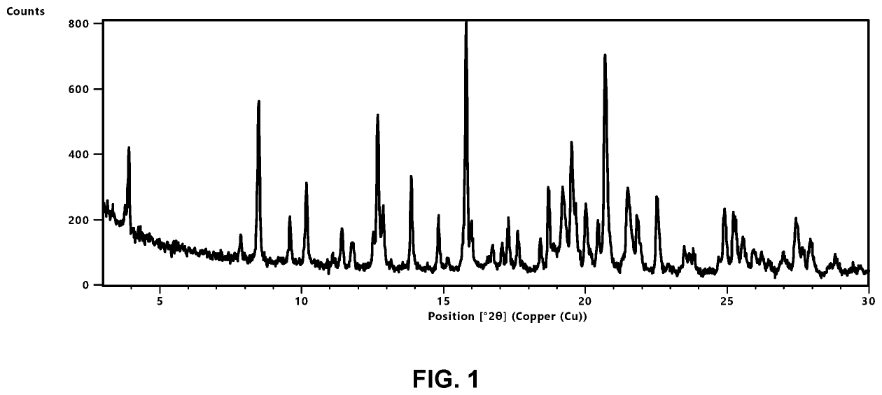

In some embodiments, the Compound I Phosphate Salt Methanol Solvate is characterized by an X-ray powder diffractogram substantially similar to that in FIG. 1.

In some embodiments, the Compound I Phosphate Salt Methanol Solvate is characterized by a ¹³C NMR spectrum comprising one or more (e.g., two or more, three or more, four or more) signals chosen from 15.7±0.2 ppm, 17.7±0.2 ppm, 40.5±0.2 ppm, 61.6±0.2 ppm, and 129.4±0.2 ppm.

In some embodiments, the Compound I Phosphate Salt Methanol Solvate is characterized by a ¹³C NMR spectrum comprising one or more (e.g., two or more, three or more, four or more) signals chosen from 15.7±0.2 ppm, 17.7±0.2 ppm, 38.9±0.2 ppm, 129.4±0.2 ppm, and 140.6±0.2 ppm.

In some embodiments, the Compound I Phosphate Salt Methanol Solvate is characterized by a ¹³C NMR spectrum comprising one or more (e.g., two or more, three or more, four or more, five or more, six or more, seven or more, eight or more, nine or more) signals chosen from 15.7±0.2 ppm, 17.7±0.2 ppm, 40.5±0.2 ppm, 47.1±0.2 ppm, 48.5±0.2 ppm, 61.6±0.2 ppm, 72.2±0.2 ppm, 73.8±0.2 ppm, 129.4±0.2 ppm, and 140.6±0.2 ppm.

In some embodiments, the Compound I Phosphate Salt Methanol Solvate is characterized by a ¹³C NMR spectrum comprising one or more (e.g., two or more, three or more, four or more, five or more, six or more, seven or more, eight or more, nine or more) signals chosen from 15.7±0.2 ppm, 17.7±0.2 ppm, 36.8±0.2 ppm, 37.7±0.2 ppm, 38.9±0.2 ppm, 127.9±0.2 ppm, 128.5±0.2 ppm, 129.4±0.2 ppm, 139.5±0.2 ppm, and 140.6±0.2 ppm.

In some embodiments, the Compound I Phosphate Salt Methanol Solvate is characterized by a ¹³C NMR spectrum comprising signals at 15.7±0.2 ppm, 17.7±0.2 ppm, 40.5±0.2 ppm, 61.6±0.2 ppm, and 129.4±0.2 ppm.

In some embodiments, the Compound I Phosphate Salt Methanol Solvate is characterized by a ¹³C NMR spectrum comprising signals at 15.7±0.2 ppm, 17.7±0.2 ppm, 38.9±0.2 ppm, 129.4±0.2 ppm, and 140.6±0.2 ppm.

In some embodiments, the Compound I Phosphate Salt Methanol Solvate is characterized by a ¹³C NMR spectrum comprising signals at 15.7±0.2 ppm, 17.7±0.2 ppm, 40.5±0.2 ppm, 47.1±0.2 ppm, 48.5±0.2 ppm, 61.6±0.2 ppm, 72.2±0.2 ppm, 73.8±0.2 ppm, 129.4±0.2 ppm, and 140.6±0.2 ppm.

In some embodiments, the Compound I Phosphate Salt Methanol Solvate is characterized by a ¹³C NMR spectrum comprising signals at 15.7±0.2 ppm, 17.7±0.2 ppm, 36.8±0.2 ppm, 37.7±0.2 ppm, 38.9±0.2 ppm, 127.9±0.2 ppm, 128.5±0.2 ppm, 129.4±0.2 ppm, 139.5±0.2 ppm, and 140.6±0.2 ppm.

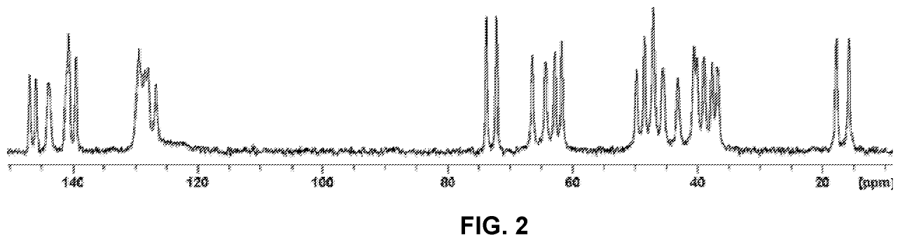

In some embodiments, the Compound I Phosphate Salt Methanol Solvate is characterized by a ¹³C NMR spectrum substantially similar to that in FIG. 2.

In some embodiments, the Compound I Phosphate Salt Methanol Solvate is characterized by a ¹⁹F NMR spectrum comprising a signal at one or more ppm values chosen from −57.7±0.2 ppm and −54.7±0.2 ppm.

In some embodiments, the Compound I Phosphate Salt Methanol Solvate is characterized by a ¹⁹F NMR spectrum comprising signals at −57.7±0.2 ppm and −54.7±0.2 ppm.

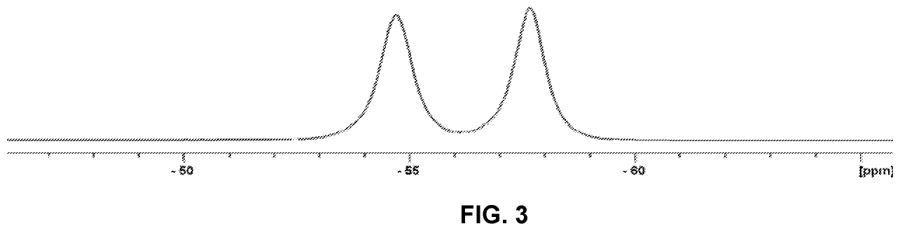

In some embodiments, the Compound I Phosphate Salt Methanol Solvate is characterized by a ¹⁹F NMR spectrum substantially similar to that in FIG. 3.

In some embodiments, the Compound I Phosphate Salt Methanol Solvate is characterized by a ³¹P NMR spectrum comprising a signal at one or more ppm values chosen from 1.8±0.2 ppm and 2.5±0.2 ppm.

In some embodiments, the Compound I Phosphate Salt Methanol Solvate is characterized by a ³¹P NMR spectrum comprising signals at 1.8±0.2 ppm and 2.5±0.2 ppm.

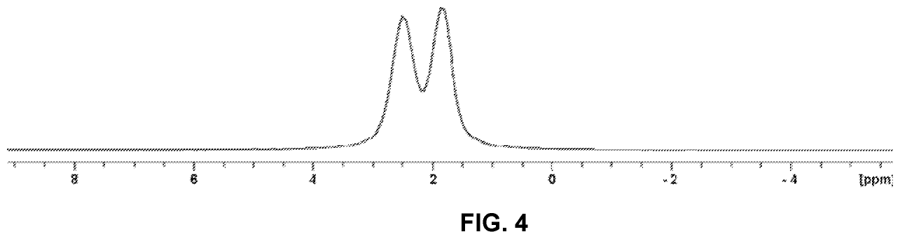

In some embodiments, the Compound I Phosphate Salt Methanol Solvate is characterized by a ³¹P NMR spectrum substantially similar to that in FIG. 4.

In some embodiments, the Compound I Phosphate Salt Methanol Solvate is characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 9.4 ± 0.1 Å | α | 90° |
| b | 10.5 ± 0.1 Å | β | 90° |
| c | 44.6 ± 0.1 Å | γ | 90°. |

Some embodiments of the disclosure provide a method of preparing Compound I Phosphate Salt Methanol Solvate comprising:

adding amorphous Compound I to MEK to create a solution;

adding 0.5M H₃PO₄ in MeOH/water to the solution;

incubating the solution at ambient temperature;

filtering the solution to isolate a solid material; and

washing the solid material.

### Compound I Phosphate Salt MEK Solvate

Some embodiments of the disclosure provide a phosphate salt MEK solvate of Compound I (Compound I Phosphate Salt MEK Solvate). In some embodiments, the Compound I Phosphate Salt MEK Solvate is substantially pure.

In some embodiments, the Compound I Phosphate Salt MEK Solvate is characterized by an X-ray powder diffractogram comprising a signal at 8.6, 15.4, and/or 20.1±0.2 two-theta.

In some embodiments, the Compound I Phosphate Salt MEK Solvate is characterized by an X-ray powder diffractogram comprising a signal at one or more two-theta values chosen from 8.6±0.2, 15.4±0.2, and 20.1±0.2. In some embodiments, the Compound I Phosphate Salt MEK Solvate is characterized by an X-ray powder diffractogram comprising a signal at two or more two-theta values chosen from 8.6±0.2, 15.4±0.2, and 20.1±0.2.

In some embodiments, the Compound I Phosphate Salt MEK Solvate is characterized by an X-ray powder diffractogram comprising a signal at the following two-theta values: 8.6±0.2, 15.4±0.2, and 20.1±0.2. In some embodiments, the Compound I Phosphate Salt MEK Solvate is characterized by an X-ray powder diffractogram comprising (a) a signal at the following two-theta values: 8.6±0.2, 15.4±0.2, and 20.1±0.2; and (b) a signal at one or more (e.g., two or more) two-theta values chosen from 15.7±0.2, 18.2±0.2, and 19.4±0.2. In some embodiments, the Compound I Phosphate Salt MEK Solvate is characterized by an X-ray powder diffractogram comprising a signal at one or more two-theta values (e.g., two or more, three or more, four or more, five or more) chosen from 8.6±0.2, 15.4±0.2, 15.7±0.2, 18.2±0.2, 19.4±0.2, and 20.1±0.2. In some embodiments, the Compound I Phosphate Salt MEK Solvate is characterized by an X-ray powder diffractogram comprising signals at the following two-theta values: 8.6±0.2, 15.4±0.2, 15.7±0.2, 18.2±0.2, 19.4±0.2, and 20.1±0.2.

In some embodiments, the Compound I Phosphate Salt MEK Solvate is characterized by an X-ray powder diffractogram comprising (a) a signal at the following two-theta values: 8.6±0.2, 15.4±0.2, and 20.1±0.2; and (b) a signal at one or more (e.g., two or more, three or more, four or more) two-theta values chosen from 15.7±0.2, 18.2±0.2, 19.4±0.2, 21.7±0.2, and 21.9±0.2. In some embodiments, the Compound I Phosphate Salt MEK Solvate is characterized by an X-ray powder diffractogram comprising a signal at one or more two-theta values (e.g., two or more, three or more, four or more, five or more, six or more, seven or more) chosen from 8.6±0.2, 15.4±0.2, 15.7±0.2, 18.2±0.2, 19.4±0.2, 20.1±0.2, 21.7±0.2, and 21.9±0.2. In some embodiments, the Compound I Phosphate Salt MEK Solvate is characterized by an X-ray powder diffractogram comprising signals at the following two-theta values: 8.6±0.2, 15.4±0.2, 15.7±0.2, 18.2 0.2, 19.4±0.2, 20.1±0.2, 21.7±0.2, and 21.9±0.2.

In some embodiments, the Compound I Phosphate Salt MEK Solvate is characterized by an X-ray powder diffractogram comprising (a) a signal at the following two-theta values: 8.6±0.2, 15.4±0.2, and 20.1±0.2; and (b) a signal at one or more (e.g., two or more, three or more, four or more, five or more, six or more) two-theta values chosen from 13.2±0.2, 15.7±0.2, 18.2±0.2, 19.4±0.2, 21.7±0.2, 21.9±0.2, and 23.8±0.2. In some embodiments, the Compound I Phosphate Salt MEK Solvate is characterized by an X-ray powder diffractogram comprising a signal at one or more two-theta values (e.g., two or more, three or more, four or more, five or more, six or more, seven or more, eight or more, nine or more) chosen from 8.6±0.2, 13.2 0.2, 15.4±0.2, 15.7±0.2, 18.2 0.2, 19.4±0.2, 20.1±0.2, 21.7±0.2, 21.9±0.2, and 23.8±0.2. In some embodiments, the Compound I Phosphate Salt MEK Solvate is characterized by an X-ray powder diffractogram comprising signals at the following two-theta values: 8.6±0.2, 13.2 0.2, 15.4±0.2, 15.7±0.2, 18.2 0.2, 19.4±0.2, 20.1±0.2, 21.7±0.2, 21.9±0.2, and 23.8±0.2.

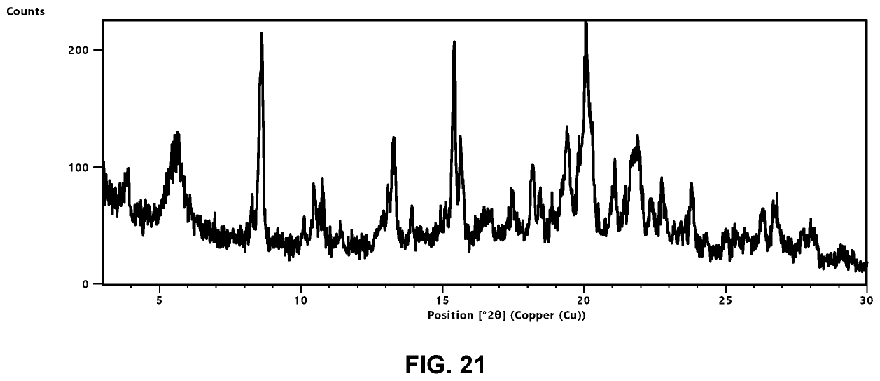

In some embodiments, the Compound I Phosphate Salt MEK Solvate is characterized by an X-ray powder diffractogram substantially similar to that in FIG. 21.

In some embodiments, the Compound I Phosphate Salt MEK Solvate is characterized by a ¹³C NMR spectrum comprising one or more (e.g., two or more, three or more, four or more) signals chosen from 16.0±0.2 ppm, 38.4±0.2 ppm, 62.3±0.2 ppm, 73.2±0.2 ppm, and 73.7±0.2 ppm.

In some embodiments, the Compound I Phosphate Salt MEK Solvate is characterized by a ¹³C NMR spectrum comprising one or more (e.g., two or more, three or more, four or more) signals chosen from 16.0±0.2 ppm, 37.5±0.2 ppm, 38.4±0.2 ppm, 126.5±0.2 ppm, and 142.0±0.2 ppm.

In some embodiments, the Compound I Phosphate Salt MEK Solvate is characterized by a ¹³C NMR spectrum comprising one or more (e.g., two or more, three or more, four or more, five or more, six or more, seven or more, eight or more, nine or more) signals chosen from 16.0±0.2 ppm, 37.5±0.2 ppm, 38.4±0.2 ppm, 47.4±0.2 ppm, 62.3±0.2 ppm, 66.3±0.2 ppm, 73.2±0.2 ppm, 73.7±0.2 ppm, 126.5±0.2 ppm, and 142.0±0.2 ppm.

In some embodiments, the Compound I Phosphate Salt MEK Solvate is characterized by a ¹³C NMR spectrum comprising one or more (e.g., two or more, three or more, four or more, five or more, six or more, seven or more, eight or more, nine or more) signals chosen from 7.4±0.2 ppm, 16.0±0.2 ppm, 36.8±0.2 ppm, 37.5±0.2 ppm, 38.4±0.2 ppm, 126.5±0.2 ppm, 128.7±0.2 ppm, 129.6±0.2 ppm, 139.4±0.2 ppm, and 142.0±0.2 ppm.

In some embodiments, the Compound I Phosphate Salt MEK Solvate is characterized by a ¹³C NMR spectrum comprising signals at 16.0±0.2 ppm, 38.4±0.2 ppm, 62.3±0.2 ppm, 73.2±0.2 ppm, and 73.7±0.2 ppm.

In some embodiments, the Compound I Phosphate Salt MEK Solvate is characterized by a ¹³C NMR spectrum comprising signals at 16.0±0.2 ppm, 37.5±0.2 ppm, 38.4±0.2 ppm, 126.5±0.2 ppm, and 142.0±0.2 ppm.

In some embodiments, the Compound I Phosphate Salt MEK Solvate is characterized by a ¹³C NMR spectrum comprising signals at 16.0±0.2 ppm, 37.5±0.2 ppm, 38.4±0.2 ppm, 47.4±0.2 ppm, 62.3±0.2 ppm, 66.3±0.2 ppm, 73.2±0.2 ppm, 73.7±0.2 ppm, 126.5±0.2 ppm, and 142.0±0.2 ppm.

In some embodiments, the Compound I Phosphate Salt MEK Solvate is characterized by a ¹³C NMR spectrum comprising signals at 7.4±0.2 ppm, 16.0±0.2 ppm, 36.8±0.2 ppm, 37.5±0.2 ppm, 38.4±0.2 ppm, 126.5±0.2 ppm, 128.7±0.2 ppm, 129.6±0.2 ppm, 139.4±0.2 ppm, and 142.0±0.2 ppm.

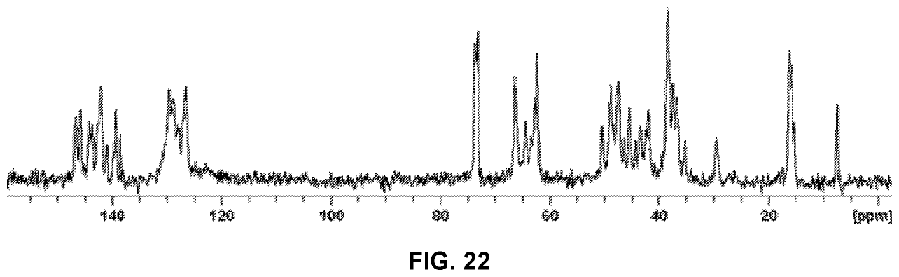

In some embodiments, the Compound I Phosphate Salt MEK Solvate is characterized by a ¹³C NMR spectrum substantially similar to that in FIG. 22.

In some embodiments, the Compound I Phosphate Salt MEK Solvate is characterized by a ¹⁹F NMR spectrum comprising a signal at one or more (e.g., two or more) ppm values chosen from −53.6±0.2 ppm, −55.2±0.2 ppm, and −57.2±0.2 ppm.

In some embodiments, the Compound I Phosphate Salt MEK Solvate is characterized by a ¹⁹F NMR spectrum comprising signals at −53.6±0.2 ppm, −55.2±0.2 ppm, and −57.2±0.2 ppm.

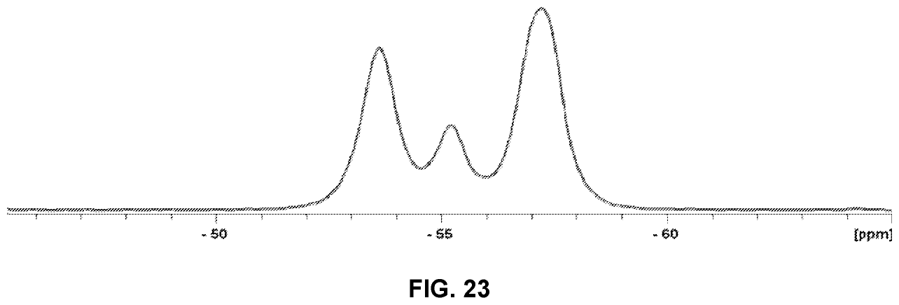

In some embodiments, the Compound I Phosphate Salt MEK Solvate is characterized by a ¹⁹F MAS spectrum substantially similar to that in FIG. 23.

In some embodiments, the Compound I Phosphate Salt MEK Solvate is characterized by a ³¹P CPMAS spectrum comprising a signal at one or more (e.g., two or more) ppm values chosen from 0.1±0.2 ppm, 2.7±0.2 ppm, and 4.8±0.2 ppm.

In some embodiments, the Compound I Phosphate Salt MEK Solvate is characterized by a ³¹P CPMAS spectrum comprising signals at 0.1±0.2 ppm, 2.7±0.2 ppm, and 4.8±0.2 ppm.

Some embodiments of the disclosure provide a method of preparing Compound I Phosphate Salt MEK Solvate comprising:
adding Compound I Phosphate Salt Hydrate Form A to MEK and mixing to form a slurry;
incubating the slurry at a reduced temperature to obtain a solid material; and centrifuging the solid material.

In some embodiments, the reduced temperature is about 5° C.

In some embodiments, incubating the slurry at a reduced temperature to obtain a solid material comprises incubating the slurry at about 5° C. for about 11 days to obtain a solid material.

### Compound I Maleate Form A (Salt or Co-Crystal)

Some embodiments of the disclosure provide a maleate salt/co-crystal form of Compound I (Compound I Maleate Form A). In some embodiments, the Compound I Maleate Form A is substantially pure.

In some embodiments, the Compound I Maleate Form A is characterized by an X-ray powder diffractogram comprising a signal at 27.6±0.2 two-theta. In some embodiments, the Compound I Maleate Form A is characterized by an X-ray powder diffractogram comprising a signal at 27.6±0.2 two-theta and 20.0±0.2 two-theta.

In some embodiments, the Compound I Maleate Form A is characterized by an X-ray powder diffractogram comprising a signal at 27.6±0.2 two-theta and a signal at one or more two-theta values chosen from 13.7±0.2, 14.5±0.2, 15.5±0.2, 18.3±0.2, and 20.0±0.2. In some embodiments, the Compound I Maleate Form A is characterized by an X-ray powder diffractogram comprising a signal at 27.6±0.2 two-theta and a signal at two or more two-theta values chosen from 13.7±0.2, 14.5±0.2, 15.5±0.2, 18.3±0.2, and 20.0±0.2. In some embodiments, the Compound I Maleate Form A is characterized by an X-ray powder diffractogram comprising a signal at 27.6±0.2 two-theta and a signal at three or more two-theta values chosen from 13.7±0.2, 14.5±0.2, 15.5±0.2, 18.3±0.2, and 20.0±0.2. In some embodiments, the Compound I Maleate Form A is characterized by an X-ray powder diffractogram comprising a signal at 27.6±0.2 two-theta and a signal at four or more two-theta values chosen from 13.7±0.2, 14.5±0.2, 15.5±0.2, 18.3±0.2, and 20.0±0.2.

In some embodiments, the Compound I Maleate Form A is characterized by an X-ray powder diffractogram comprising signals at 27.6±0.2 two-theta, 13.7±0.2 two-theta, 14.5±0.2 two-theta, 15.5±0.2 two-theta, 18.3±0.2 two-theta, and 20.0±0.2 two-theta.

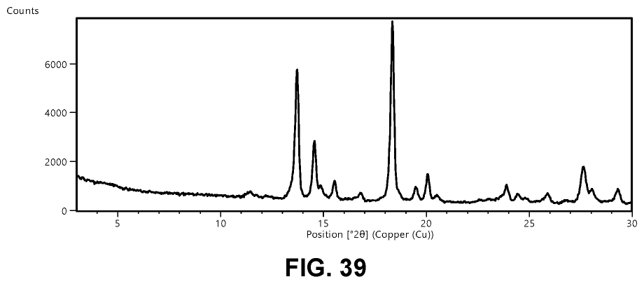

In some embodiments, the Compound I Maleate Form A (salt or co-crystal) is characterized by an X-ray powder diffractogram substantially similar to that in FIG. 39.

In some embodiments, the Compound I Maleate Form A (salt or co-crystal) is characterized by a TGA thermogram showing minimal weight loss until degradation.

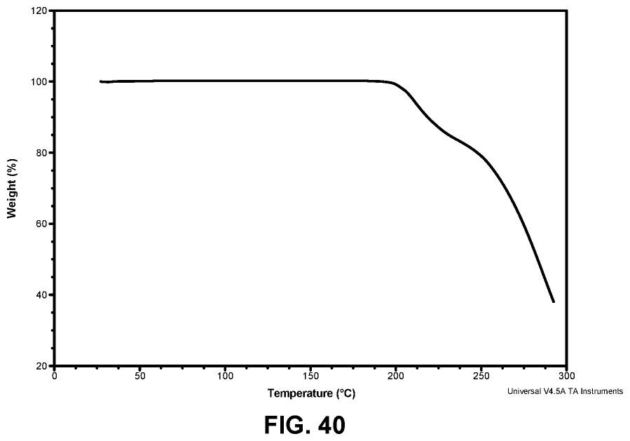

In some embodiments, the Compound I Maleate Form A (salt or co-crystal) is characterized by a TGA thermogram substantially similar to that in FIG. 40.

In some embodiments, the Compound I Maleate Form A (salt or co-crystal) is characterized by a DSC curve having an endothermic peak at about 201° C.

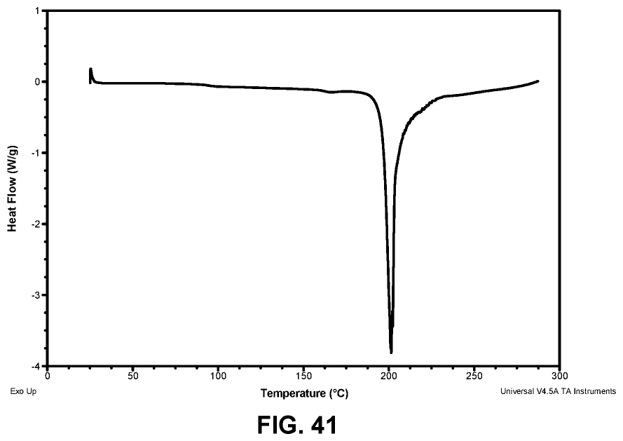

In some embodiments, the Compound I Maleate Form A (salt or co-crystal) is characterized by a DSC curve substantially similar to that in FIG. 41.

Some embodiments of the disclosure provide a method of preparing Compound I Maleate Form A (salt or co-crystal) comprising:

dissolving Compound I Monohydrate in acetonitrile;

adding maleic acid to form a suspension and stirring at ambient temperature for 3 days;

centrifuging the suspension and air drying the resulting wet cake; and

heating to 165° C. and isolating the solids.

### Compound I Maleate Form B (Salt or Co-Crystal)

Some embodiments of the disclosure provide a second maleate salt/co-crystal form of Compound I (Compound I Maleate Form B). In some embodiments, the Compound I Maleate Form B is substantially pure.

In some embodiments, the Compound I Maleate Form B is characterized by an X-ray powder diffractogram comprising a signal at 4.9±0.2 two-theta. In some embodiments, the Compound I Maleate Form B is characterized by an X-ray powder diffractogram comprising a signal at 26.0±0.2 two-theta. In some embodiments, the Compound I Maleate Form B is characterized by an X-ray powder diffractogram comprising a signal at 4.9±0.2 two-theta and 26.0±0.2 two-theta.

In some embodiments, the Compound I Maleate Form B is characterized by an X-ray powder diffractogram comprising (a) a signal at 4.9±0.2 two-theta and/or a signal at 26.0±0.2 two-theta; and (b) a signal at one or more two-theta values chosen from 13.8±0.2, 14.7±0.2, 15.4±0.2, 18.3±0.2, and 19.6±0.2. In some embodiments, the Compound I Maleate Form B is characterized by an X-ray powder diffractogram comprising (a) a signal at 4.9±0.2 two-theta and/or a signal at 26.0±0.2 two-theta; and (b) a signal at two or more two-theta values chosen from 13.8±0.2, 14.7±0.2, 15.4±0.2, 18.3±0.2, and 19.6±0.2. In some embodiments, the Compound I Maleate Form B is characterized by an X-ray powder diffractogram comprising (a) a signal at 4.9±0.2 two-theta and/or a signal at 26.0±0.2 two-theta; and (b) a signal at three or more two-theta values chosen from 13.8±0.2, 14.7±0.2, 15.4±0.2, 18.3±0.2, and 19.6±0.2. In some embodiments, the Compound I Maleate Form B is characterized by an X-ray powder diffractogram comprising (a) a signal at 4.9±0.2 two-theta and/or a signal at 26.0±0.2 two-theta; and (b) a signal at four or more two-theta values chosen from 13.8±0.2, 14.7±0.2, 15.4±0.2, 18.3±0.2, and 19.6±0.2.

In some embodiments, the Compound I Maleate Form B is characterized by an X-ray powder diffractogram comprising signals at 4.9±0.2 two-theta, 13.8±0.2 two-theta, 14.7±0.2 two-theta, 15.4±0.2 two-theta, 18.3±0.2 two-theta, 19.6±0.2 two-theta, and 26.0±0.2 two-theta.

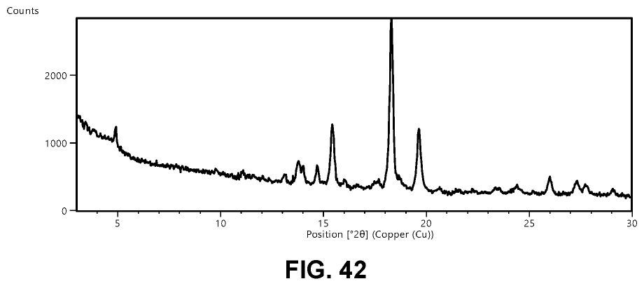

In some embodiments, the Compound I Maleate Form B (salt or co-crystal) is characterized by an X-ray powder diffractogram substantially similar to that in FIG. 42.

In some embodiments, the Compound I Maleate Form B (salt or co-crystal) is characterized by a TGA thermogram showing minimal weight loss until degradation.

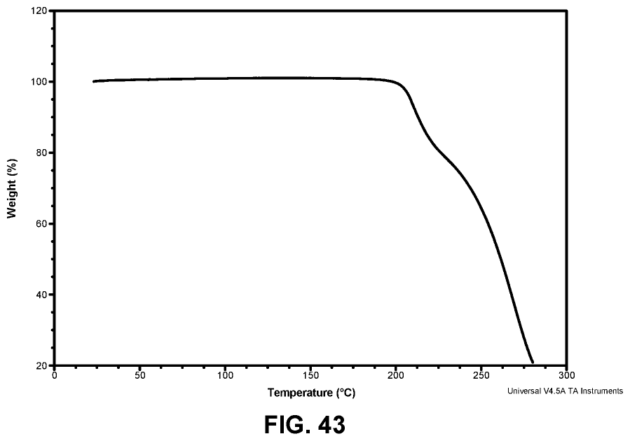

In some embodiments, the Compound I Maleate Form B (salt or co-crystal) is characterized by a TGA thermogram substantially similar to that in FIG. 43.

In some embodiments, the Compound I Maleate Form B (salt or co-crystal) is characterized by a DSC curve having an endothermic peak at about 206° C.

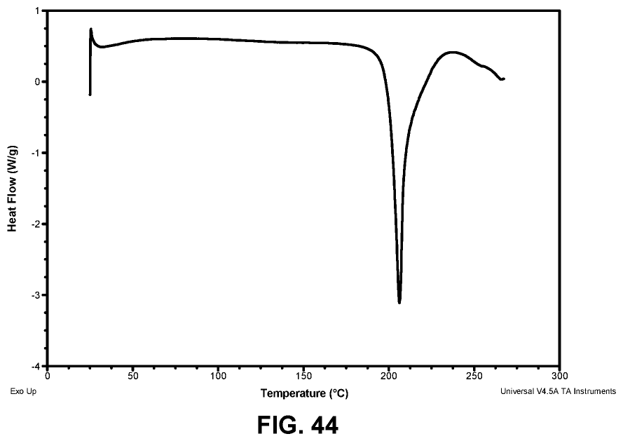

In some embodiments, the Compound I Maleate Form B (salt or co-crystal) is characterized by a DSC curve substantially similar to that in FIG. 44.

Some embodiments of the disclosure provide a method of preparing Compound I Maleate Form B (salt or co-crystal) comprising:

dissolving Compound I Monohydrate in ethanol;

adding maleic acid and stirring at ambient temperature for 3 days;

fast evaporating for 5 days; and

heating to 150° C. and isolating the solids.

### Compound I Fumaric Acid Form A (Salt or Co-Crystal)

Some embodiments of the disclosure provide a fumaric acid salt/co-crystal form of Compound I (Compound I Fumaric Acid Form A). In some embodiments, the Compound I Fumaric Acid Form A is substantially pure.

In some embodiments, the Compound I Fumaric Acid Form A is characterized by an X-ray powder diffractogram comprising a signal at 21.5±0.2 two-theta. In some embodiments, the Compound I Fumaric Acid Form A is characterized by an X-ray powder diffractogram comprising a signal at 14.4±0.2 two-theta. In some embodiments, the Compound I Fumaric Acid Form A is characterized by an X-ray powder diffractogram comprising a signal at 14.6±0.2 two-theta. In some embodiments, the Compound I Fumaric Acid Form A is characterized by an X-ray powder diffractogram comprising a signal at 16.9±0.2 two-theta. In some embodiments, the Compound I Fumaric Acid Form A is characterized by an X-ray powder diffractogram comprising a signal at 20.7±0.2 two-theta. In some embodiments, the Compound I Fumaric Acid Form A is characterized by an X-ray powder diffractogram comprising a signal at 20.9±0.2 two-theta.

In some embodiments, the Compound I Fumaric Acid Form A is characterized by an X-ray powder diffractogram comprising a signal at two or more two-theta values chosen from 14.4±0.2, 14.6±0.2, 16.9±0.2, 20.7±0.2, 20.9±0.2, and 21.5±0.2. In some embodiments, the Compound I Fumaric Acid Form A is characterized by an X-ray powder diffractogram comprising a signal at three or more two-theta values chosen from 14.4±0.2, 14.6±0.2, 16.9±0.2, 20.7±0.2, 20.9±0.2, and 21.5±0.2. In some embodiments, the Compound I Fumaric Acid Form A is characterized by an X-ray powder diffractogram comprising a signal at four or more two-theta values chosen from 14.4±0.2, 14.6±0.2, 16.9±0.2, 20.7±0.2, 20.9±0.2, and 21.5±0.2. In some embodiments, the Compound I Fumaric Acid Form A is characterized by an X-ray powder diffractogram comprising a signal at five or more two-theta values chosen from 14.4±0.2, 14.6±0.2, 16.9±0.2, 20.7±0.2, 20.9±0.2, and 21.5±0.2. In some embodiments, the Compound I Fumaric Acid Form A is characterized by an X-ray powder diffractogram comprising signals at 14.4±0.2 two-theta, 14.6±0.2 two-theta, 16.9±0.2 two-theta, 20.7±0.2 two-theta, 20.9±0.2 two-theta, and 21.5±0.2 two-theta.

In some embodiments, the Compound I Fumaric Acid Form A is characterized by an X-ray powder diffractogram comprising (a) a signal at 21.5±0.2 two-theta and/or a signal at 16.9±0.2 two-theta; and (b) a signal at one, two, three, four, five, six, seven, eight, nine, ten, or more two-theta values chosen from 9.5±0.2, 14.4±0.2, 14.6±0.2, 15.6±0.2, 16.9±0.2, 17.3±0.2, 17.5±0.2, 19.1±0.2, 19.5±0.2, 19.7±0.2, 20.7±0.2, 20.9±0.2, 21.0±0.2, 22.5±0.2, 23.2±0.2, 25.7±0.2, 28.3±0.2, and 29.4±0.2.

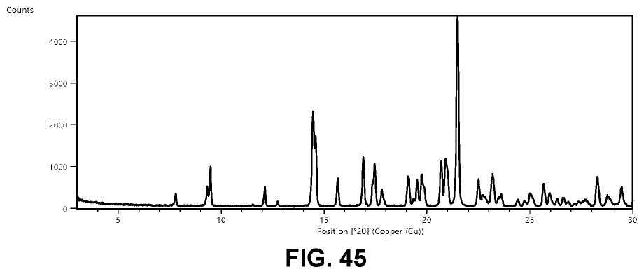

In some embodiments, the Compound I Fumaric Acid Form A (salt or co-crystal) is characterized by an X-ray powder diffractogram substantially similar to that in FIG. 45.

In some embodiments, the Compound I Fumaric Acid Form A (salt or co-crystal) is characterized by a TGA thermogram showing minimal weight loss from ambient temperature up to 100° C.

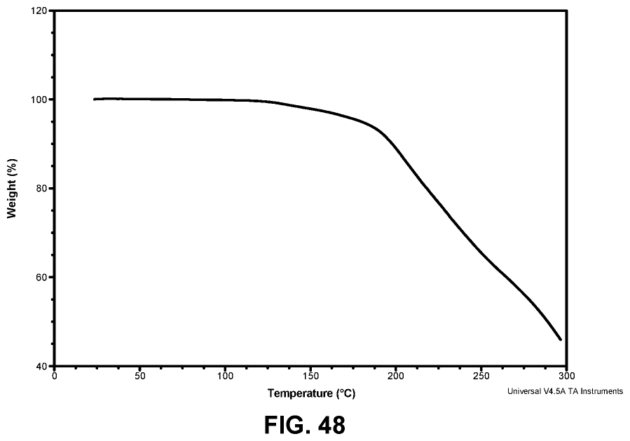

In some embodiments, the Compound I Fumaric Acid Form A (salt or co-crystal) is characterized by a TGA thermogram substantially similar to that in FIG. 48.

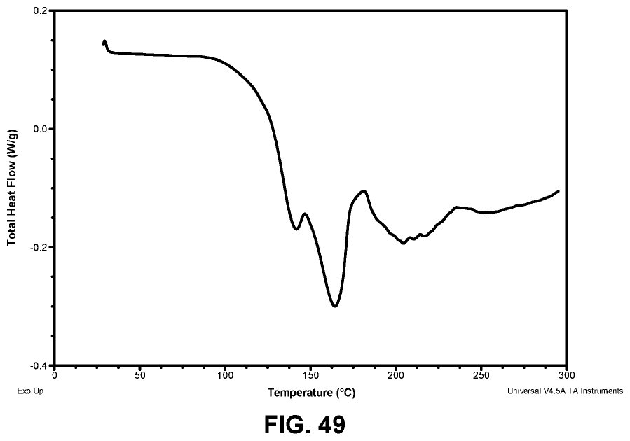

In some embodiments, the Compound I Fumaric Acid Form A (salt or co-crystal) is characterized by a DSC curve having two endothermic peaks at about 137° C. and 165° C. In some embodiments, the Compound I Fumaric Acid Form A (salt or co-crystal) is characterized by a DSC curve substantially similar to that in FIG. 49.

In some embodiments, the Compound I Fumaric Acid Form A is characterized by a ¹³C NMR spectrum comprising one or more signals chosen from 172.4±0.2 ppm, 128.1±0.2 ppm, 72.9±0.2 ppm, and 17.2±0.2 ppm. In some embodiments, the Compound I Fumaric Acid Form A is characterized by a ¹³C NMR spectrum comprising two or more signals chosen from 172.4±0.2 ppm, 128.1±0.2 ppm, 72.9±0.2 ppm, and 17.2±0.2 ppm. In some embodiments, the Compound I Fumaric Acid Form A is characterized by a ¹³C NMR spectrum comprising three or more signals chosen from 172.4±0.2 ppm, 128.1±0.2 ppm, 72.9±0.2 ppm, and 17.2±0.2 ppm. In some embodiments, the Compound I Fumaric Acid Form A is characterized by a ¹³C NMR spectrum comprising signals at 172.4±0.2 ppm, 128.1±0.2 ppm, 72.9±0.2 ppm, and 17.2±0.2 ppm.

In some embodiments, the Compound I Fumaric Acid Form A is characterized by a ¹³C NMR spectrum comprising one or more (e.g., two or more, three or more, four or more, etc.) signals chosen from 172.4±0.2 ppm, 171.4±0.2 ppm, 148.4±0.2 ppm, 143.8±0.2 ppm, 142.1±0.2 ppm, 135.5±0.2 ppm, 130.7±0.2 ppm, 128.1±0.2 ppm, 127.3±0.2 ppm, 124.3±0.2 ppm, 121.5±0.2 ppm, 72.9±0.2 ppm, 65.7±0.2 ppm, 61.8±0.2 ppm, 50.8±0.2 ppm, 48.3±0.2 ppm, 47.3±0.2 ppm, 42.0±0.2 ppm, 38.3±0.2 ppm, 34.6±0.2 ppm, and 17.2±0.2 ppm.

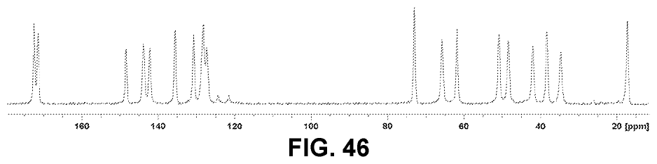

In some embodiments, the Compound I Fumaric Acid Form A is characterized by a ¹³C NMR spectrum substantially similar to that in FIG. 46.

In some embodiments, the Compound I Fumaric Acid Form A (salt or co-crystal) is characterized by a ¹⁹F MAS spectrum comprising a single signal at −55.8±0.2 ppm.

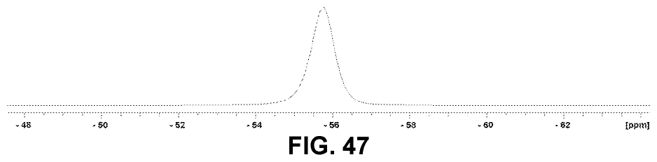

In some embodiments, the Compound I Fumaric Acid Form A (salt or co-crystal) is characterized by a ¹⁹F MAS spectrum substantially similar to that in FIG. 47.

Some embodiments of the disclosure provide a method of preparing Compound I Fumaric Acid Form A (salt or co-crystal) comprising:

adding a vial containing ceramic beads and water to a high through-put ball-mill containing a 3:4 ratio of Compound I Monohydrate and fumaric acid;

running ball mill for three cycles of 60 seconds with 10 second pauses between cycles;

placing in a vacuum oven at 45° C. overnight; and

isolating the solids.

### Compound I Free Form Form B

Some embodiments of the disclosure provide a free form of Compound I (Compound I Form B). In some embodiments, the Compound I free form Form B is substantially pure.

In some embodiments, the Compound I free form Form B is characterized by an X-ray powder diffractogram comprising a signal at 21.6±0.2 two-theta. In some embodiments, the Compound I free form Form B is characterized by an X-ray powder diffractogram comprising a signal at 13.9±0.2 two-theta. In some embodiments, the Compound I free form Form B is characterized by an X-ray powder diffractogram comprising a signal at 19.1±0.2 two-theta. In some embodiments, the Compound I free form Form B is characterized by an X-ray powder diffractogram comprising a signal at 11.7±0.2 two-theta. In some embodiments, the Compound I free form Form B is characterized by an X-ray powder diffractogram comprising a signal at 14.2±0.2 two-theta. In some embodiments, the Compound I free form Form B is characterized by an X-ray powder diffractogram comprising a signal at 24.6±0.2 two-theta.

In some embodiments, the Compound I free form Form B is characterized by an X-ray powder diffractogram comprising a signal at two or more two-theta values chosen from 11.7±0.2, 13.9±0.2, 14.2±0.2, 19.1±0.2, 21.6±0.2, and 24.6±0.2. In some embodiments, the Compound I free form Form B is characterized by an X-ray powder diffractogram comprising a signal at three or more two-theta values chosen from 11.7±0.2, 13.9±0.2, 14.2±0.2, 19.1±0.2, 21.6±0.2, and 24.6±0.2. In some embodiments, the Compound I free form Form B is characterized by an X-ray powder diffractogram comprising a signal at four or more two-theta values chosen from 11.7±0.2, 13.9±0.2, 14.2±0.2, 19.1±0.2, 21.6±0.2, and 24.6±0.2. In some embodiments, the Compound I free form Form B is characterized by an X-ray powder diffractogram comprising a signal at five or more two-theta values chosen from 11.7±0.2, 13.9±0.2, 14.2±0.2, 19.1±0.2, 21.6±0.2, and 24.6±0.2. In some embodiments, the Compound I free form Form B is characterized by an X-ray powder diffractogram comprising signals at 11.7±0.2 two-theta, 13.9±0.2 two-theta, 14.2±0.2 two-theta, 19.1±0.2 two-theta, 21.6±0.2 two-theta, and 24.6±0.2 two-theta.

In some embodiments, the Compound I free form Form B is characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) a signal at one or more two-theta values chosen from 13.1±0.2, 20.6±0.2, 17.5±0.2, 15.8±0.2, and 18.9±0.2. In some embodiments, the Compound I free form Form B is characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, and 20.6±0.2 two-theta. In some embodiments, the Compound I free form Form B is characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, 20.6±0.2 two-theta, and 17.5±0.2 two-theta. In some embodiments, the Compound I free form Form B is characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, 20.6±0.2 two-theta, 17.5±0.2 two-theta, and 15.8±0.2 two-theta. In some embodiments, the Compound I free form Form B is characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, 20.6±0.2 two-theta, 17.5±0.2 two-theta, 15.8±0.2 two-theta, and 18.9±0.2 two-theta.

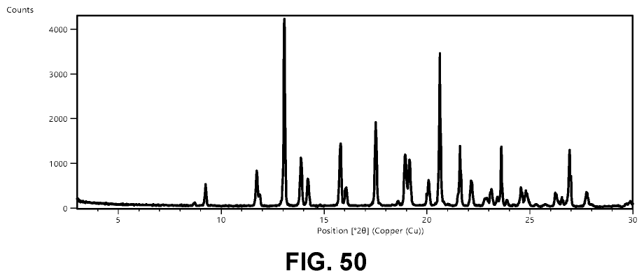

In some embodiments, the Compound I free form Form B is characterized by an X-ray powder diffractogram substantially similar to that in FIG. 50.

In some embodiments, the Compound I free form Form B is characterized by a TGA thermogram showing minimal weight loss from ambient temperature up to 180° C.

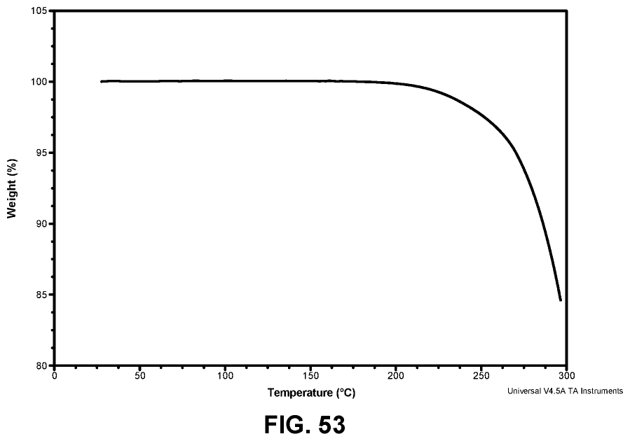

In some embodiments, the Compound I free form Form B is characterized by a TGA thermogram substantially similar to that in FIG. 53.

In some embodiments, the Compound I free form Form B is characterized by a DSC curve having a broad endothermic peak at about 132° C.

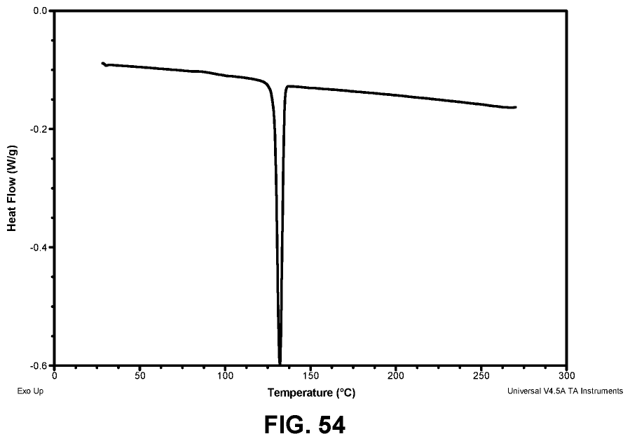

In some embodiments, the Compound I free form Form B is characterized by a DSC curve substantially similar to that in FIG. 54.

In some embodiments, the Compound I free form Form B is characterized by a ¹³C NMR spectrum comprising one or more signals chosen from 152.2±0.2 ppm, 148.1±0.2 ppm, and 140.0±0.2 ppm. In some embodiments, the Compound I free form Form B is characterized by a ¹³C NMR spectrum comprising one or more signals chosen from 73.7±0.2 ppm, 47.9±0.2 ppm, and 23.5±0.2 ppm. In some embodiments, the Compound I free form Form B is characterized by a ¹³C NMR spectrum comprising (a) one or more signals chosen from 152.2±0.2 ppm, 148.1±0.2 ppm, and 140.0±0.2 ppm and (b) one or more signals chosen from 73.7±0.2 ppm, 47.9±0.2 ppm, and 23.5±0.2 ppm.

In some embodiments, the Compound I free form Form B is characterized by a ¹³C NMR spectrum comprising signals at 152.2±0.2 ppm, 148.1±0.2 ppm, and 140.0±0.2 ppm. In some embodiments, the Compound I free form Form B is characterized by a ¹³C NMR spectrum comprising signals at 73.7±0.2 ppm, 47.9±0.2 ppm, and 23.5±0.2 ppm. In some embodiments, the Compound I free form Form B is characterized by a ¹³C NMR spectrum comprising signals at 152.2±0.2 ppm, 148.1±0.2 ppm, 140.0±0.2 ppm, 73.7±0.2 ppm, 47.9±0.2 ppm, and 23.5±0.2 ppm.

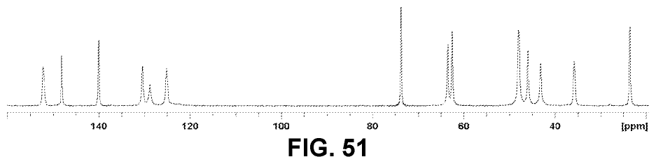

In some embodiments, the Compound I free form Form B is characterized by a ¹³C NMR spectrum substantially similar to that in FIG. 51.

In some embodiments, the Compound I free form Form B is characterized by a ¹⁹F MAS spectrum comprising a single signal at −54.8±0.2 ppm.

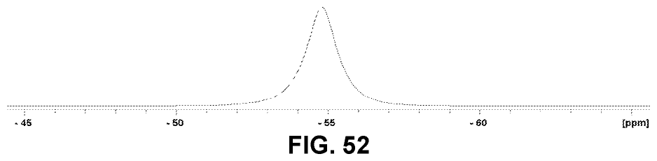

In some embodiments, the Compound I free form Form B is characterized by a ¹⁹F MAS spectrum substantially similar to that in FIG. 52.

In some embodiments, the Compound I free form Form B is characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 8.1 ± 0.1 Å | α | 90° |
| b | 11.8 ± 0.1 Å | β | 90° |
| c | 18.9 ± 0.1 Å | γ | 90°. |

In some embodiments, the Compound I free form Form B is characterized by an orthorhombic crystal system, a P2₁2₁2₁ space group, and unit cell dimensions measured at 298 K on a Bruker diffractometer equipped Cu Kα radiation (λ=1.54178 Å) of:

| | | | |
|---|---|---|---|
| a | 8.2 ± 0.1 Å | α | 90° |
| b | 11.9 ± 0.1 Å | β | 90° |
| c | 19.1 ± 0.1 Å | γ | 90°. |

Some embodiments of the disclosure provide a method A (method A) of preparing Compound I free form Form B comprising:

heating Compound I free form Monohydrate to 120° C. for two hours;

cooling to 90° C. and maintaining at 90° C. for 5 days; and

isolating the solid Compound I free form Form B.

Some embodiments of the disclosure provide an alternate method (method B) of preparing Compound I free form Form B comprising:

placing amorphous free form Compound I in heptane vapor for 5 days; and

isolating solid Compound I free form Form B.

### Compound I Free Form Form C

Some embodiments of the disclosure provide a free form of Compound I (Compound I free form Form C). In some embodiments, the Compound I free form Form C is substantially pure.

In some embodiments, the Compound I free form Form C is characterized by an X-ray powder diffractogram comprising a signal at 11.1±0.2 two-theta. In some embodiments, the Compound I free form Form C is characterized by an X-ray powder diffractogram comprising a signal at 25.7±0.2 two-theta. In some embodiments, the Compound I free form Form C is characterized by an X-ray powder diffractogram comprising a signal at 14.7±0.2 two-theta. In some embodiments, the Compound I free form Form C is characterized by an X-ray powder diffractogram comprising a signal at 21.0±0.2 two-theta.

In some embodiments, the Compound I free form Form C is characterized by an X-ray powder diffractogram comprising a signal at two or more two-theta values chosen from 11.1±0.2, 14.7±0.2, 21.0±0.2, and 25.7±0.2. In some embodiments, the Compound I free form Form C is characterized by an X-ray powder diffractogram comprising a signal at three or more two-theta values chosen from 11.1±0.2, 14.7±0.2, 21.0±0.2, and 25.7±0.2. In some embodiments, the Compound I free form Form C is characterized by an X-ray powder diffractogram comprising signals at 11.1±0.2 two-theta, 14.7±0.2 two-theta, 21.0±0.2, and 25.7±0.2 two-theta.

In some embodiments, the Compound I free form Form C is characterized by an X-ray powder diffractogram comprising (a) a signal at one or more two-theta values chosen from 11.1±0.2, 14.7±0.2, 21.0±0.2, and 25.7±0.2; and (b) a signal at one or more two-theta values chosen from 9.5±0.2, 17.7±0.2, 12.9±0.2, 15.4±0.2, 18.6±0.2, and 25.9±0.2. In some embodiments, the Compound I free form Form C is characterized by an X-ray powder diffractogram comprising (a) a signal at one or more two-theta values chosen from 11.1±0.2, 14.7±0.2, 21.0±0.2, and 25.7±0.2; and (b) a signal at two or more two-theta values chosen from 9.5±0.2, 17.7±0.2, 12.9±0.2, 15.4±0.2, 18.6±0.2, and 25.9±0.2. In some embodiments, the Compound I free form Form C is characterized by an X-ray powder diffractogram comprising (a) a signal at one or more two-theta values chosen from 11.1±0.2, 14.7±0.2, 21.0±0.2, and 25.7±0.2; and (b) a signal at three or more two-theta values chosen from 9.5±0.2, 17.7±0.2, 12.9±0.2, 15.4±0.2, 18.6±0.2, and 25.9±0.2. In some embodiments, the Compound I free form Form C is characterized by an X-ray powder diffractogram comprising (a) a signal at one or more two-theta values chosen from 11.1±0.2, 14.7±0.2, 21.0±0.2, and 25.7±0.2; and (b) a signal at four or more two-theta values chosen from 9.5±0.2, 17.7±0.2, 12.9±0.2, 15.4±0.2, 18.6±0.2, and 25.9±0.2. In some embodiments, the Compound I free form Form C is characterized by an X-ray powder diffractogram comprising (a) a signal at one or more two-theta values chosen from 11.1±0.2, 14.7±0.2, 21.0±0.2, and 25.7±0.2; and (b) signals at 17.7±0.2 two-theta, 12.9±0.2 two-theta, 15.4±0.2 two-theta, and 18.6±0.2 two-theta.

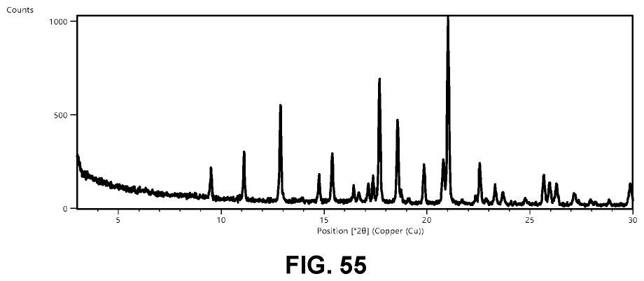

In some embodiments, the Compound I free form Form C is characterized by an X-ray powder diffractogram substantially similar to that in FIG. 55.

In some embodiments, the Compound I free form Form C is characterized by a TGA thermogram showing minimal weight loss from ambient temperature up to 190° C.

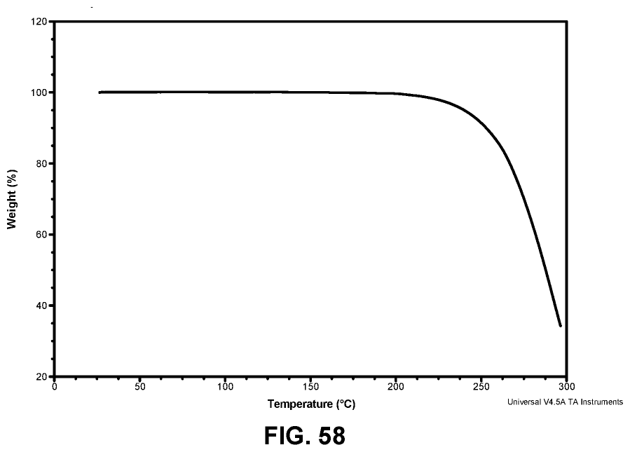

In some embodiments, the Compound I free form Form C is characterized by a TGA thermogram substantially similar to that in FIG. 58.

In some embodiments, the Compound I free form Form C is characterized by a DSC curve having an endothermic peak at about 134° C.

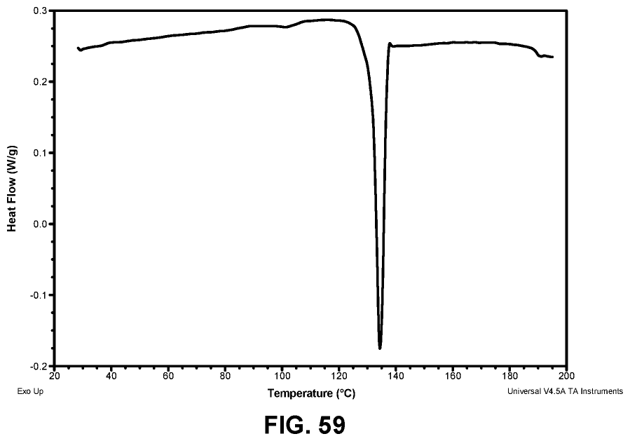

In some embodiments, the Compound I free form Form C is characterized by a DSC curve substantially similar to that in FIG. 59.

In some embodiments, the Compound I free form Form C is characterized by a ¹³C NMR spectrum comprising one or more signals chosen from 149.6±0.2 ppm, 149.2±0.2 ppm, and 137.1±0.2 ppm. In some embodiments, the Compound I free form Form C is characterized by a ¹³C NMR spectrum comprising one or more signals chosen from 74.5±0.2 ppm, 62.4±0.2 ppm, 48.3±0.2 ppm, and 24.6±0.2 ppm. In some embodiments, the Compound I free form Form C is characterized by a ¹³C NMR spectrum comprising (a) one or more signals chosen from 149.6±0.2 ppm, 149.2±0.2 ppm, and 137.1±0.2 ppm; and (b) one or more signals chosen from 74.5±0.2 ppm, 62.4±0.2 ppm, 48.3±0.2 ppm, and 24.6±0.2 ppm.

In some embodiments, the Compound I free form Form C is characterized by a ¹³C NMR spectrum comprising signals at 149.6±0.2 ppm, 149.2±0.2 ppm, and 137.1±0.2 ppm. In some embodiments, the Compound I free form Form C is characterized by a ¹³C NMR spectrum comprising signals at 74.5±0.2 ppm, 62.4±0.2 ppm, 48.3±0.2 ppm, and 24.6±0.2 ppm. In some embodiments, the Compound I free form Form C is characterized by a ¹³C NMR spectrum comprising signals at 149.6±0.2 ppm, 149.2±0.2 ppm, 137.1±0.2 ppm, 74.5±0.2 ppm, 62.4±0.2 ppm, 48.3±0.2 ppm, and 24.6±0.2 ppm.

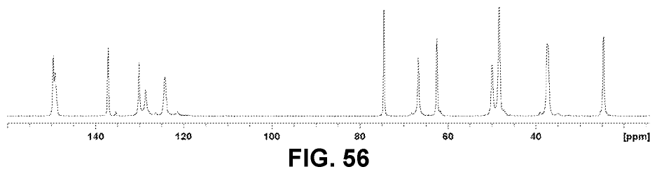

In some embodiments, the Compound I free form Form C is characterized by a ¹³C NMR spectrum substantially similar to that in FIG. 56.

In some embodiments, the Compound I free form Form C is characterized by a ¹⁹F MAS spectrum comprising a single signal at −54.0±0.2 ppm.

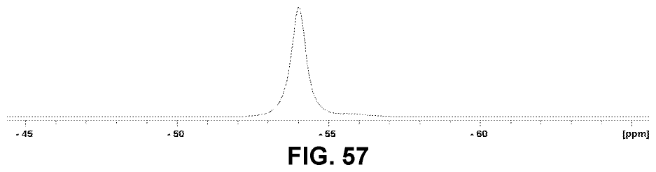

In some embodiments, the Compound I free form Form C is characterized by a ¹⁹F MAS spectrum substantially similar to that in FIG. 57.

In some embodiments, the Compound I free form Form C is characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 10.1 ± 0.1 Å | α | 90° |
| b | 12.5 ± 0.1 Å | β | 90° |
| c | 13.4 ± 0.1 Å | γ | 90°. |

Some embodiments of the disclosure provide a method (method A) of preparing Compound I free form Form C comprising:

obtaining a seed of Compound I free form Form C by thermal treatment on a physical mixture Compound I free form monohydrate and Compound II free form Form C in a TGA pan;

thermal treating with TGA ramping at 10° C. per min to 120° C., isothermal at 120° C. for 60 minutes, and then cooling at 2° C. per min down to 25° C.;

adding the seed produced with this thermal treatment into a Compound I free form monohydrate heptane slurry and maintaining at 50° C. for 7 days; and

isolating the solid Compound I free form Form C.

Some embodiments of the disclosure provide an alternate method (method B) of preparing Compound I free form Form C comprising:

charging Compound I free form Monohydrate and Heptane, Ethyl Acetate to a reactor;

agitating and heating the slurry to 65° C.;

seeding with Compound I free form Form C;

agitating and isolating at 65° C. for 3 days; and

isolating solids and vacuum drying under nitrogen blanket at 50° C.

### Compound II Phosphate Salt Hemihydrate Form A

Some embodiments of the disclosure provide a phosphate hemihydrate of Compound II (Compound II Phosphate Salt Hemihydrate Form A). In some embodiments, the Compound II Phosphate Salt Hemihydrate Form A is substantially pure.

In some embodiments, the Compound II Phosphate Salt Hemihydrate Form A is characterized by an X-ray powder diffractogram comprising a signal at 9.1 two-theta, 16.7 two-theta, and/or 18.7±0.2 two-theta.

In some embodiments, the Compound II Phosphate Salt Hemihydrate Form A is characterized by an X-ray powder diffractogram comprising a signal at one or more two-theta values chosen from 9.1±0.2, 16.7±0.2, and 18.7±0.2. In some embodiments, the Compound II Phosphate Salt Hemihydrate Form A is characterized by an X-ray powder diffractogram comprising a signal at two or more two-theta values chosen from 9.1±0.2, 16.7±0.2, and 18.7±0.2.

In some embodiments, the Compound II Phosphate Salt Hemihydrate Form A is characterized by an X-ray powder diffractogram comprising a signal at the following two-theta values: 9.1±0.2, 16.7±0.2, and 18.7±0.2. In some embodiments, the Compound II Phosphate Salt Hemihydrate Form A is characterized by an X-ray powder diffractogram comprising (a) a signal at the following two-theta values: 9.1±0.2, 16.7±0.2, and 18.7±0.2; and (b) a signal at one or more two-theta (e.g., two or more) values chosen from 14.9±0.2, 15.7±0.2, and 20.0±0.2. In some embodiments, the Compound II Phosphate Salt Hemihydrate Form A is characterized by an X-ray powder diffractogram comprising a signal at one or more two-theta values (e.g., two or more, three or more, four or more, five or more) chosen from 9.1±0.2, 14.9±0.2, 15.7±0.2, 16.7±0.2, 18.7±0.2, and 20.0±0.2. In some embodiments, the Compound II Phosphate Salt Hemihydrate Form A is characterized by an X-ray powder diffractogram comprising signals at the following two-theta values: 9.1±0.2, 14.9±0.2, 15.7±0.2, 16.7±0.2, 18.7±0.2, and 20.0±0.2.

In some embodiments, the Compound II Phosphate Salt Hemihydrate Form A is characterized by an X-ray powder diffractogram comprising (a) a signal at the following two-theta values: 9.1±0.2, 16.7±0.2, and 18.7±0.2; and (b) a signal at one or more (e.g., two or more, three or more, four or more) two-theta values chosen from 10.1±0.2, 14.9±0.2, 15.7±0.2, 18.4±0.2, and 20.0±0.2. In some embodiments, the Compound II Phosphate Salt Hemihydrate Form A is characterized by an X-ray powder diffractogram comprising a signal at one or more two-theta values (e.g., two or more, three or more, four or more, five or more, six or more, seven or more) chosen from 9.1±0.2, 10.1±0.2, 14.9±0.2, 15.7±0.2, 16.7±0.2, 18.4±0.2, 18.7±0.2, and 20.0±0.2. In some embodiments, the Compound II Phosphate Salt Hemihydrate Form A is characterized by an X-ray powder diffractogram comprising signals at the following two-theta values: 9.1±0.2, 10.1±0.2, 14.9±0.2, 15.7±0.2, 16.7±0.2, 18.4±0.2, 18.7±0.2, and 20.0±0.2.

In some embodiments, the Compound II Phosphate Salt Hemihydrate Form A is characterized by an X-ray powder diffractogram comprising (a) a signal at the following two-theta values: 9.1±0.2, 16.7±0.2, and 18.7±0.2; and (b) a signal at one or more (e.g., two or more, three or more, four or more, five or more, six or more) two-theta values chosen from 10.1±0.2, 14.9±0.2, 15.2 0.2, 15.7±0.2, 18.4±0.2, 20.0±0.2, and 20.2±0.2. In some embodiments, the Compound II Phosphate Salt Hemihydrate Form A is characterized by an X-ray powder diffractogram comprising a signal at one or more two-theta values (e.g., two or more, three or more, four or more, five or more, six or more, seven or more, eight or more, nine or more) chosen from 9.1±0.2, 10.1±0.2, 14.9±0.2, 15.7±0.2, 16.7±0.2, 18.4±0.2, 18.7±0.2, and 20.0±0.2. In some embodiments, the Compound II Phosphate Salt Hemihydrate Form A is characterized by an X-ray powder diffractogram comprising signals at the following two-theta values: 9.1±0.2, 10.1±0.2, 14.9±0.2, 15.2±0.2, 15.7±0.2, 16.7±0.2, 18.4±0.2, 18.7±0.2, 20.0±0.2, and 20.2±0.2.

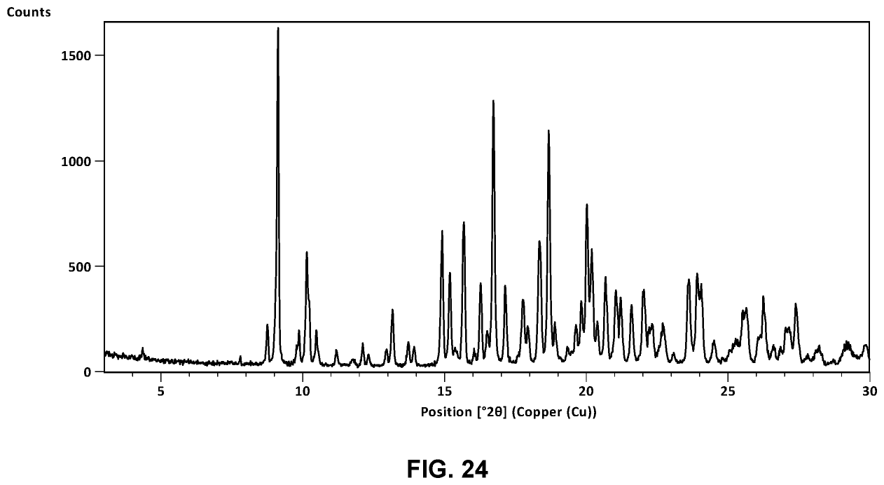

In some embodiments, the Compound II Phosphate Salt Hemihydrate Form A is characterized by an X-ray powder diffractogram substantially similar to that in FIG. 24.

In some embodiments, the Compound II Phosphate Salt Hemihydrate Form A is characterized by a ¹³C NMR spectrum comprising one or more (e.g., two or more, three or more, four or more) signals chosen from 47.7±0.2 ppm, 50.5±0.2 ppm, 72.5±0.2 ppm, 73±0.2 ppm, and 141.3±0.2 ppm.

In some embodiments, the Compound II Phosphate Salt Hemihydrate Form A is characterized by a ¹³C NMR spectrum comprising one or more (e.g., two or more, three or more, four or more) signals chosen from 15.3±0.2 ppm, 15.8±0.2 ppm, 16.6±0.2 ppm, 39.9±0.2 ppm, and 141.3±0.2 ppm.

In some embodiments, the Compound II Phosphate Salt Hemihydrate Form A is characterized by a ¹³C NMR spectrum comprising one or more (e.g., two or more, three or more, four or more, five or more, six or more, seven or more, eight or more, nine or more) signals chosen from 15.3±0.2 ppm, 16.6±0.2 ppm, 46.9±0.2 ppm, 47.7±0.2 ppm, 50.5±0.2 ppm, 63.4±0.2 ppm, 65.4±0.2 ppm, 72.5±0.2 ppm, 73±0.2 ppm, and 141.3±0.2 ppm.

In some embodiments, the Compound II Phosphate Salt Hemihydrate Form A is characterized by a ¹³C NMR spectrum comprising one or more (e.g., two or more, three or more, four or more, five or more, six or more, seven or more, eight or more, nine or more) signals chosen from 15.3±0.2 ppm, 15.8±0.2 ppm, 16.6±0.2 ppm, 18.4±0.2 ppm, 38.6±0.2 ppm, 39.9±0.2 ppm, 126.6±0.2 ppm, 127.1±0.2 ppm, 136.8±0.2 ppm, and 141.3±0.2 ppm.

In some embodiments, the Compound II Phosphate Salt Hemihydrate Form A is characterized by a ¹³C NMR spectrum comprising signals at 47.7±0.2 ppm, 50.5±0.2 ppm, 72.5±0.2 ppm, 73±0.2 ppm, and 141.3±0.2 ppm.

In some embodiments, the Compound II Phosphate Salt Hemihydrate Form A is characterized by a ¹³C NMR spectrum comprising signals at 15.3±0.2 ppm, 15.8±0.2 ppm, 16.6±0.2 ppm, 39.9±0.2 ppm, and 141.3±0.2 ppm.

In some embodiments, the Compound II Phosphate Salt Hemihydrate Form A is characterized by a ¹³C NMR spectrum comprising signals at 15.3±0.2 ppm, 16.6±0.2 ppm, 46.9±0.2 ppm, 47.7±0.2 ppm, 50.5±0.2 ppm, 63.4±0.2 ppm, 65.4±0.2 ppm, 72.5±0.2 ppm, 73±0.2 ppm, and 141.3±0.2 ppm.

In some embodiments, the Compound II Phosphate Salt Hemihydrate Form A is characterized by a ¹³C NMR spectrum comprising signals at 15.3±0.2 ppm, 15.8±0.2 ppm, 16.6±0.2 ppm, 18.4±0.2 ppm, 38.6±0.2 ppm, 39.9±0.2 ppm, 126.6±0.2 ppm, 127.1±0.2 ppm, 136.8±0.2 ppm, and 141.3±0.2 ppm.

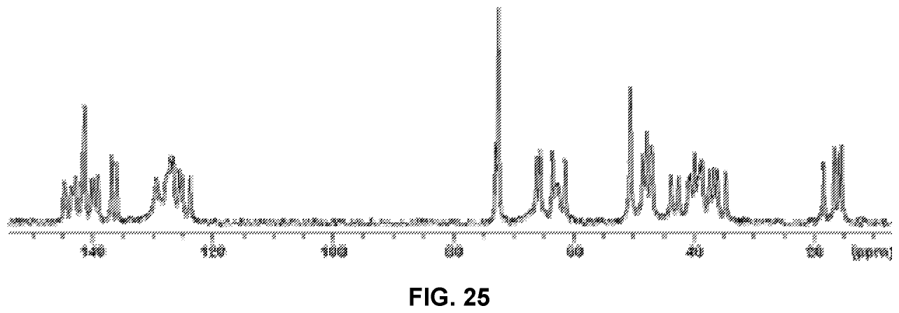

In some embodiments, the Compound II Phosphate Salt Hemihydrate Form A is characterized by a ¹³C NMR spectrum substantially similar to that in FIG. 25.

In some embodiments, the Compound II Phosphate Salt Hemihydrate Form A is characterized by a ¹³C NMR spectrum measured after dehydration comprising one or more (e.g., two or more, three or more, four or more) signals chosen from 16.5±0.2 ppm, 48.5±0.2 ppm, 66.5±0.2 ppm, 72.2±ppm, and 73.3±0.2 ppm.

In some embodiments, the Compound II Phosphate Salt Hemihydrate Form A is characterized by a ¹³C NMR spectrum measured after dehydration comprising one or more (e.g., two or more, three or more, four or more) signals chosen from 16.5±0.2 ppm, 38.5±0.2 ppm, 39.3±0.2 ppm, 125.6±ppm, and 127.5±0.2 ppm.

In some embodiments, the Compound II Phosphate Salt Hemihydrate Form A is characterized by a ¹³C NMR spectrum measured after dehydration comprising one or more (e.g., two or more, three or more, four or more, five or more, six or more, seven or more, eight or more, nine or more) signals chosen from 16.5±0.2 ppm, 38.5±0.2 ppm, 39.3±0.2 ppm, 48.5±0.2 ppm, 64.1±0.2 ppm, 66.5±0.2 ppm, 72.2±0.2 ppm, 73±0.2 ppm, 73.3±0.2 ppm, and 127.5±0.2 ppm.

In some embodiments, the Compound II Phosphate Salt Hemihydrate Form A is characterized by a ¹³C NMR spectrum measured after dehydration comprising one or more (e.g., two or more, three or more, four or more, five or more, six or more, seven or more, eight or more, nine or more) signals chosen from 16.5±0.2 ppm, 36.6±0.2 ppm, 37±0.2 ppm, 38.5±0.2 ppm, 39.3±0.2 ppm, 125.6±0.2 ppm, 127.5±0.2 ppm, 136.8±0.2 ppm, 141.3±0.2 ppm, and 143±0.2 ppm.

In some embodiments, the Compound II Phosphate Salt Hemihydrate Form A is characterized by a ¹³C NMR spectrum measured after dehydration comprising signals at 16.5±0.2 ppm, 48.5±0.2 ppm, 66.5±0.2 ppm, 72.2±ppm, and 73.3±0.2 ppm.

In some embodiments, the Compound II Phosphate Salt Hemihydrate Form A is characterized by a ¹³C NMR spectrum measured after dehydration comprising signals at 16.5±0.2 ppm, 38.5±0.2 ppm, 39.3±0.2 ppm, 125.6±ppm, and 127.5±0.2 ppm.

In some embodiments, the Compound II Phosphate Salt Hemihydrate Form A is characterized by a ¹³C NMR spectrum measured after dehydration comprising signals at 16.5±0.2 ppm, 38.5±0.2 ppm, 39.3±0.2 ppm, 48.5±0.2 ppm, 64.1±0.2 ppm, 66.5±0.2 ppm, 72.2±0.2 ppm, 73±0.2 ppm, 73.3±0.2 ppm, and 127.5±0.2 ppm.

In some embodiments, the Compound II Phosphate Salt Hemihydrate Form A is characterized by a ¹³C NMR spectrum measured after dehydration comprising signals at 16.5±0.2 ppm, 36.6±0.2 ppm, 37±0.2 ppm, 38.5±0.2 ppm, 39.3±0.2 ppm, 125.6±0.2 ppm, 127.5±0.2 ppm, 136.8±0.2 ppm, 141.3±0.2 ppm, and 143±0.2 ppm.

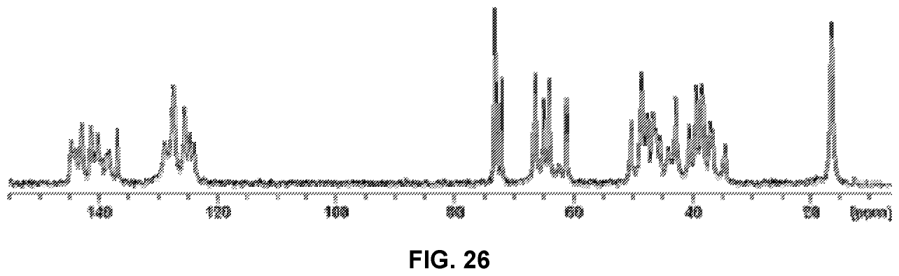

In some embodiments, the Compound II Phosphate Salt Hemihydrate Form A is characterized by a ¹³C NMR spectrum measured after dehydration substantially similar to that in FIG. 26.

In some embodiments, the Compound II Phosphate Salt Hemihydrate Form A is characterized by a ³¹P NMR spectrum comprising a signal at one or more (e.g., two or more) ppm values chosen from −1.8±0.2 ppm, −1.1±0.2 ppm, and 3.1±0.2 ppm.

In some embodiments, the Compound II Phosphate Salt Hemihydrate Form A is characterized by a ³¹P NMR spectrum comprising signals at −1.8±0.2 ppm, −1.1±0.2 ppm, and 3.1±0.2 ppm.

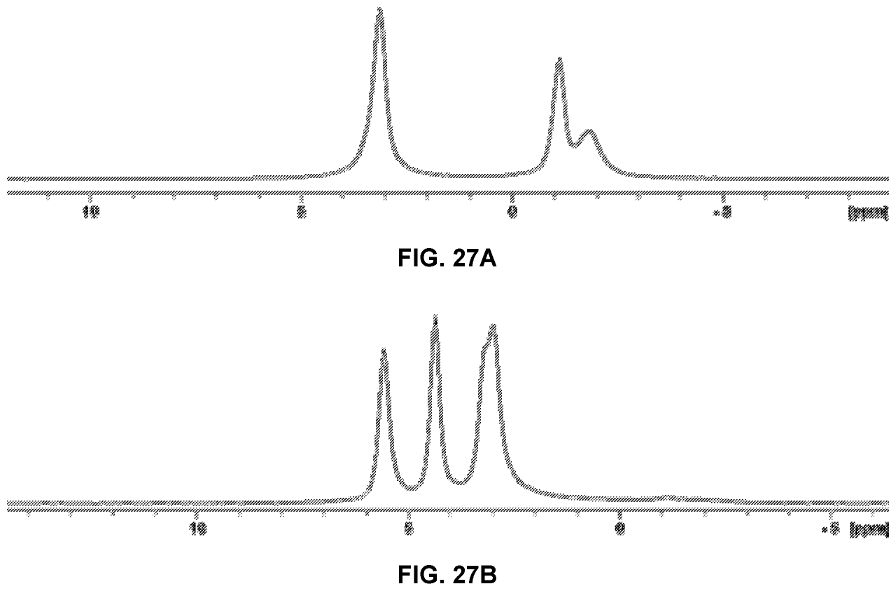

In some embodiments, the Compound II Phosphate Salt Hemihydrate Form A is characterized by a ³¹P NMR spectrum substantially similar to that in FIG. 27A.

In some embodiments, the Compound II Phosphate Salt Hemihydrate Form A is characterized by a ³¹P NMR spectrum measured after dehydration comprising a signal at one or more (e.g., two or more, three or more) ppm values chosen from 3.0±0.2 ppm, 3.2±0.2 ppm, 4.4±0.2 ppm, and 5.6±0.2 ppm.

In some embodiments, the Compound II Phosphate Salt Hemihydrate Form A is characterized by a ³¹P NMR spectrum measured after dehydration comprising signals at 3.0±0.2 ppm, 3.2±0.2 ppm, 4.4±0.2 ppm, and 5.6±0.2 ppm.

In some embodiments, the Compound II Phosphate Salt Hemihydrate Form A is characterized by a ³¹P NMR spectrum measured after dehydration substantially similar to that in FIG. 27B.

In some embodiments, the Compound II Phosphate Salt Hemihydrate Form A is characterized by a TGA thermogram showing 2.4% weight loss from ambient temperature to 150° C.

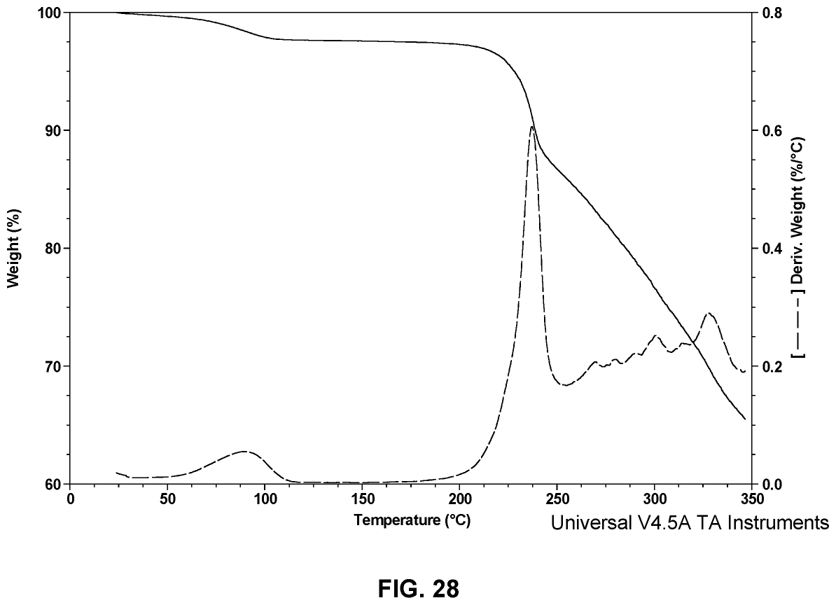

In some embodiments, the Compound II Phosphate Salt Hemihydrate Form A is characterized by a TGA thermogram substantially similar to that in FIG. 28.

In some embodiments, the Compound II Phosphate Salt Hemihydrate Form A is characterized by a DSC curve having endothermic peaks at about 123° C. and at about 224° C.

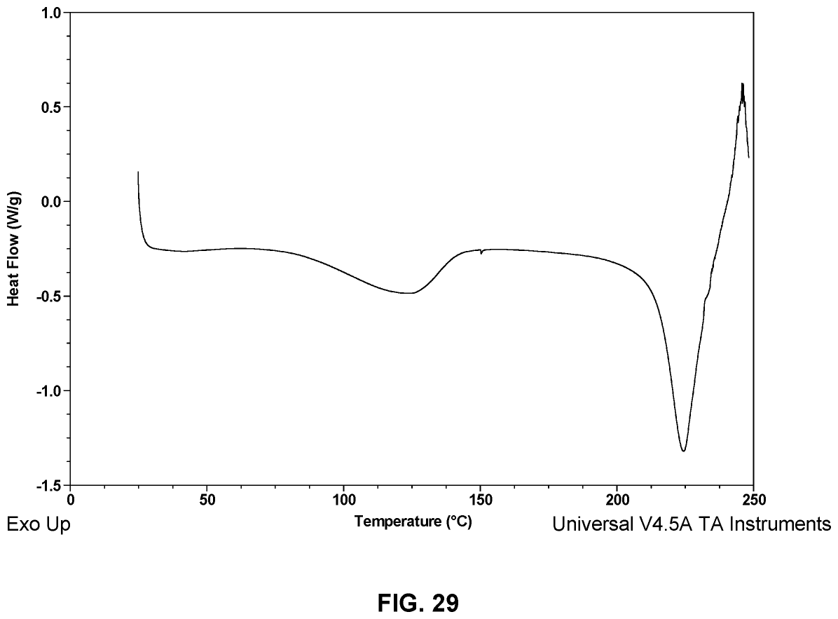

In some embodiments, the Compound II Phosphate Salt Hemihydrate Form A is characterized by a DSC curve substantially similar to that in FIG. 29.

In some embodiments, the Compound II Phosphate Salt Hemihydrate Form A is characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 9.2 ± 0.1 Å | α | 90° |
| b | 23.5 ± 0.1 Å | β | 90° |
| c | 38.3 ± 0.1 Å | γ | 90°. |

Some embodiments of the disclosure provide a method of preparing Compound II Phosphate Salt Hemihydrate Form A comprising:

adding Compound II free form Hemihydrate Form A to 2-MeTHF to form a solution;

adding H₃PO₄ dropwise to the solution;

stirring the solution at ambient temperature;

collecting a solid material by centrifugation; and

drying the solid material.

In some embodiments, stirring the solution at ambient temperature comprises stirring the solution at ambient temperature for about 2 days.

In some embodiments, drying the solid material comprises drying the solid material in a vacuum oven at about 40° C. overnight.

Some embodiments of the disclosure provide a method of preparing Compound II Phosphate Salt Hemihydrate Form A comprising:

charging Compound II free form Hemihydrate and 2-MeTHF to a reactor;

agitating the reactor at about 40° C.;

seeding the reactor with Compound II Phosphate Salt Hemihydrate Form A;

slowly adding a phosphoric acid solution to the reactor to form a slurry;

cooling the slurry; and

agitating the cooled slurry and filtering under vacuum to yield a wet cake; and

drying the wet cake.

In some embodiments, cooling the slurry comprises cooling the slurry to about 20° C.

In some embodiments, cooling the slurry comprises cooling the slurry to about 20° C. over about 5 hours.

In some embodiments, agitating the cooled slurry comprises agitating the cooled slurry at about 20° C. for at least about 2 hours.

### Compound II Free Form Hemihydrate Form A

Some embodiments of the disclosure provide a hemihydrate of Compound II (Compound II free form Hemihydrate Form A). In some embodiments, the Compound II free form Hemihydrate Form A is substantially pure.

In some embodiments, the Compound II free form Hemihydrate Form A is characterized by an X-ray powder diffractogram measured at ambient temperature comprising a signal at 17.1, 19.1, and/or 20.4±0.2 two-theta.

In some embodiments, the Compound II free form Hemihydrate Form A is characterized by an X-ray powder diffractogram measured at ambient temperature comprising a signal at one or more two-theta values chosen from 17.1±0.2, 19.1±0.2, and 20.4±0.2. In some embodiments, the Compound II free form Hemihydrate Form A is characterized by an X-ray powder diffractogram measured at ambient temperature comprising a signal at two or more two-theta values chosen 17.1±0.2, 19.1±0.2, and 20.4±0.2.

In some embodiments, the Compound II free form Hemihydrate Form A is characterized by an X-ray powder diffractogram measured at ambient temperature comprising a signal at the following two-theta values: 17.1±0.2, 19.1±0.2, and 20.4±0.2. In some embodiments, the Compound II free form Hemihydrate Form A is characterized by an X-ray powder diffractogram measured at ambient temperature comprising (a) a signal at the following two-theta values: 17.1±0.2, 19.1±0.2, and 20.4±0.2; and (b) a signal at one or more two-theta values chosen from 5.7±0.2, 6.5±0.2, and 14.4±0.2. In some embodiments, the Compound II free form Hemihydrate Form A is characterized by an X-ray powder diffractogram measured at ambient temperature comprising a signal at one or more two-theta values (e.g., two or more, three or more, four or more, five or more) chosen from 5.7±0.2, 6.5±0.2, 14.4±0.2, 17.1±0.2, 19.1±0.2, and 20.4±0.2. In some embodiments, the Compound II free form Hemihydrate Form A is characterized by an X-ray powder diffractogram measured at ambient temperature comprising signals at the following two-theta values: 5.7±0.2, 6.5±0.2, 14.4±0.2, 17.1±0.2, 19.1±0.2, and 20.4±0.2.

In some embodiments, the Compound II free form Hemihydrate Form A is characterized by an X-ray powder diffractogram measured at ambient temperature comprising (a) a signal at the following two-theta values: 17.1±0.2, 19.1±0.2, and 20.4±0.2; and (b) a signal at one or more two-theta values chosen from 5.7±0.2, 6.5±0.2, 11.4±0.2, 12.1±0.2, and 14.4±0.2. In some embodiments, the Compound II free form Hemihydrate Form A is characterized by an X-ray powder diffractogram measured at ambient temperature comprising a signal at one or more two-theta values (e.g., two or more, three or more, four or more, five or more, six or more, seven or more) chosen from 5.7±0.2, 6.5±0.2, 11.4±0.2, 12.1±0.2, 14.4±0.2, 17.1±0.2, 19.1±0.2, and 20.4±0.2. In some embodiments, the Compound II free form Hemihydrate Form A is characterized by an X-ray powder diffractogram measured at ambient temperature comprising signals at the following two-theta values: 5.7±0.2, 6.5±0.2, 11.4±0.2, 12.1±0.2, 14.4±0.2, 17.1±0.2, 19.1±0.2, and 20.4±0.2.

In some embodiments, the Compound II free form Hemihydrate Form A is characterized by an X-ray powder diffractogram measured at ambient temperature comprising (a) a signal at the following two-theta values: 17.1±0.2, 19.1±0.2, and 20.4±0.2; and (b) a signal at one or more two-theta values chosen from 5.7±0.2, 6.5±0.2, 11.4±0.2, 12.1±0.2, 12.3±0.2, 14.4±0.2, and 25.5±0.2. In some embodiments, the Compound II free form Hemihydrate Form A is characterized by an X-ray powder diffractogram measured at ambient temperature comprising a signal at one or more two-theta values (e.g., two or more, three or more, four or more, five or more, six or more, seven or more, eight or more, nine or more) chosen from 5.7±0.2, 6.5±0.2, 11.4±0.2, 12.1±0.2, 12.3±0.2, 14.4±0.2, 17.1±0.2, 19.1±0.2, 20.4±0.2, and 25.5±0.2. In some embodiments, the Compound II free form Hemihydrate Form A is characterized by an X-ray powder diffractogram measured at ambient temperature comprising signals at the following two-theta values: 5.7±0.2, 6.5±0.2, 11.4±0.2, 12.1±0.2, 12.3±0.2, 14.4±0.2, 17.1±0.2, 19.1±0.2, 20.4±0.2, and 25.5±0.2.

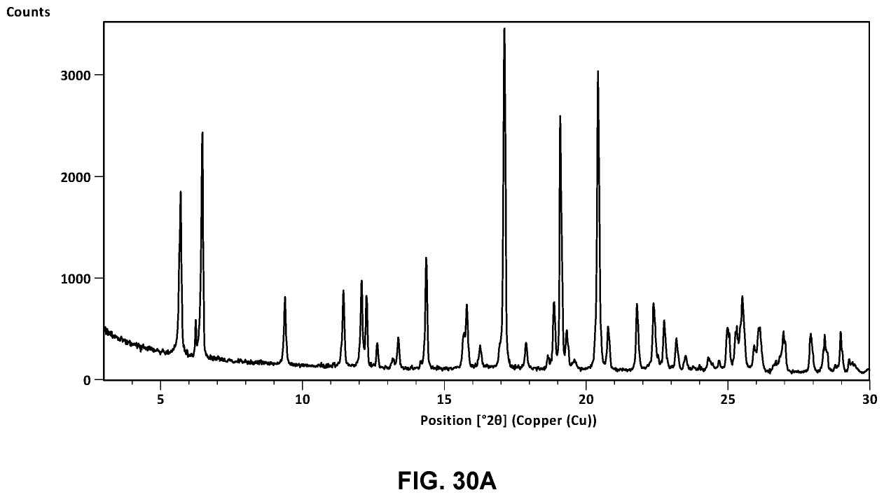

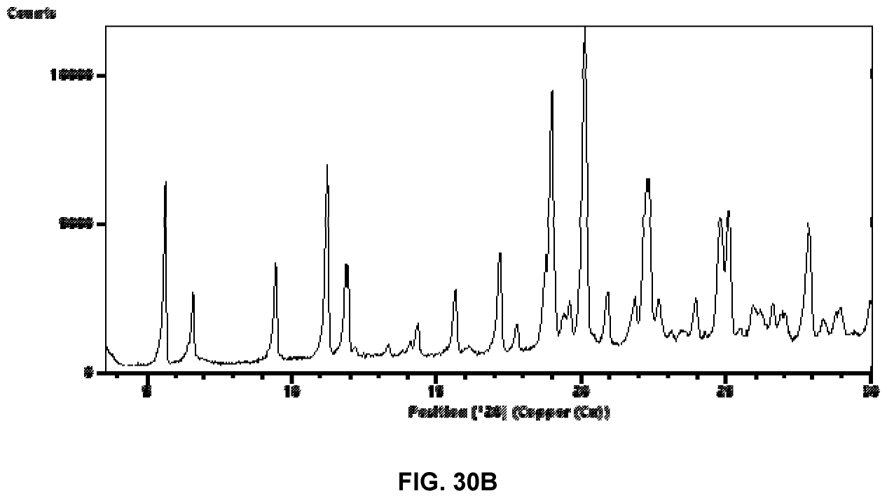

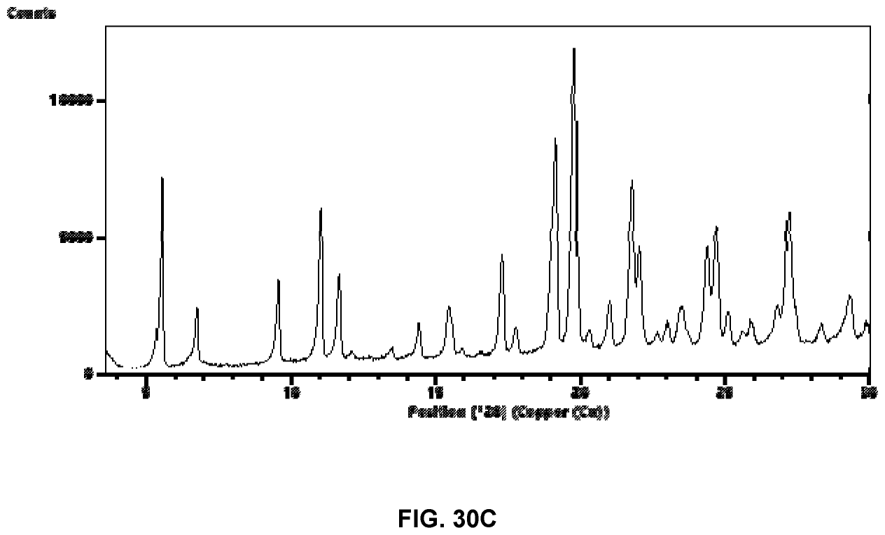

In some embodiments, the Compound II free form Hemihydrate Form A is characterized by an X-ray powder diffractogram measured at ambient temperature substantially similar to that in FIG. 30A.

In some embodiments, the Compound II free form Hemihydrate Form A is characterized by an X-ray powder diffractogram measured at a temperature in the range of 40° C. to 50° C. comprising a signal at 11.3, 19.0, and/or 20.1±0.2 two-theta.

In some embodiments, the Compound II free form Hemihydrate Form A is characterized by an X-ray powder diffractogram measured at a temperature in the range of 40° C. to 50° C. comprising a signal at one or more two-theta values chosen from 11.3±0.2, 19.0±0.2, and 20.1±0.2. In some embodiments, the Compound II free form Hemihydrate Form A is characterized by an X-ray powder diffractogram measured at a temperature in the range of 40° C. to 50° C. comprising a signal at two or more two-theta values chosen from 11.3±0.2, 19.0±0.2, and 20.1±0.2.

In some embodiments, the Compound II free form Hemihydrate Form A is characterized by an X-ray powder diffractogram measured at a temperature in the range of 40° C. to 50° C. comprising a signal at the following two-theta values: 11.3±0.2, 19.0±0.2, and 20.1±0.2. In some embodiments, the Compound II free form Hemihydrate Form A is characterized by an X-ray powder diffractogram measured at a temperature in the range of 40° C. to 50° C. comprising (a) a signal at the following two-theta values: 11.3±0.2, 19.0±0.2, and 20.1±0.2; and (b) a signal at one or more two-theta values chosen from 5.6±0.2, 22.3±0.2, and 25.1±0.2. In some embodiments, the Compound II free form Hemihydrate Form A is characterized by an X-ray powder diffractogram measured at a temperature in the range of 40° C. to 50° C. comprising a signal at one or more two-theta values (e.g., two or more, three or more, four or more, five or more) chosen from 5.6±0.2, 11.3±0.2, 19.0±0.2, 20.1±0.2, 22.3±0.2, and 25.1±0.2. In some embodiments, the Compound II free form Hemihydrate Form A is characterized by an X-ray powder diffractogram measured at a temperature in the range of 40° C. to 50° C. comprising signals at the following two-theta values: 5.6±0.2, 11.3±0.2, 19.0±0.2, 20.1±0.2, 22.3±0.2, and 25.1±0.2.

In some embodiments, the Compound II free form Hemihydrate Form A is characterized by an X-ray powder diffractogram measured at a temperature in the range of 40° C. to 50° C. comprising (a) a signal at the following two-theta values: 11.3±0.2, 19.0±0.2, and 20.1±0.2; and (b) a signal at one or more two-theta (e.g., two or more, three or more, four or more) values chosen from 5.6±0.2, 22.3±0.2, 24.8±0.2, 25.1±0.2, and 27.8±0.2. In some embodiments, the Compound II free form Hemihydrate Form A is characterized by an X-ray powder diffractogram measured at a temperature in the range of 40° C. to 50° C. comprising a signal at one or more two-theta values (e.g., two or more, three or more, four or more, five or more, six or more, seven or more) chosen from 5.6±0.2, 11.3±0.2, 19.0±0.2, 20.1±0.2, 22.3±0.2, 24.8±0.2, 25.1±0.2, and 27.8±0.2. In some embodiments, the Compound II free form Hemihydrate Form A is characterized by an X-ray powder diffractogram measured at a temperature in the range of 40° C. to 50° C. comprising signals at the following two-theta values: 5.6±0.2, 11.3±0.2, 19.0±0.2, 20.1±0.2, 22.3±0.2, 24.8±0.2, 25.1±0.2, and 27.8±0.2.

In some embodiments, the Compound II free form Hemihydrate Form A is characterized by an X-ray powder diffractogram measured at a temperature in the range of 40° C. to 50° C. comprising (a) a signal at the following two-theta values: 11.3±0.2, 19.0±0.2, and 20.1±0.2; and (b) a signal at one or more two-theta (e.g., two or more, three or more, four or more, five or more, six or more) values chosen from 5.6±0.2, 17.2±0.2, 22.1±0.2, 22.3±0.2, 24.8±0.2, 25.1±0.2, and 27.8±0.2. In some embodiments, the Compound II free form Hemihydrate Form A is characterized by an X-ray powder diffractogram measured at a temperature in the range of 40° C. to 50° C. comprising a signal at one or more two-theta values (e.g., two or more, three or more, four or more, five or more, six or more, seven or more, eight or more, nine or more) chosen from 5.6±0.2, 11.3±0.2, 17.2±0.2, 19.0±0.2, 20.1±0.2, 22.1±0.2, 22.3±0.2, 24.8±0.2, 25.1±0.2, and 27.8±0.2. In some embodiments, the Compound II free form Hemihydrate Form A is characterized by an X-ray powder diffractogram measured at a temperature in the range of 40° C. to 50° C. comprising signals at the following two-theta values: 5.6±0.2, 11.3±0.2, 17.2 0.2, 19.0±0.2, 20.1±0.2, 22.1±0.2, 22.3±0.2, 24.8±0.2, 25.1±0.2, and 27.8±0.2.

In some embodiments, the Compound II free form Hemihydrate Form A is characterized by an X-ray powder diffractogram measured at a temperature in the range of 40° C. to 50° C. substantially similar to that in FIG. 30B.

In some embodiments, the Compound II free form Hemihydrate Form A is characterized by an X-ray powder diffractogram measured at a temperature in the range of 60° C. to 90° C. comprising a signal at 5.5, 19.2, and/or 19.8±0.2 two-theta.

In some embodiments, the Compound II free form Hemihydrate Form A is characterized by an X-ray powder diffractogram measured at a temperature in the range of 60° C. to 90° C. comprising a signal at one or more two-theta values chosen from 5.5±0.2, 19.2±0.2, and 19.8±0.2. In some embodiments, the Compound II free form Hemihydrate Form A is characterized by an X-ray powder diffractogram measured at a temperature in the range of 60° C. to 90° C. comprising a signal at two or more two-theta values chosen from 5.5±0.2, 19.2±0.2, and 19.8±0.2.

In some embodiments, the Compound II free form Hemihydrate Form A is characterized by an X-ray powder diffractogram measured at a temperature in the range of 60° C. to 90° C. comprising a signal at the following two-theta values: 5.5±0.2, 19.2±0.2, and 19.8±0.2. In some embodiments, the Compound II free form Hemihydrate Form A is characterized by an X-ray powder diffractogram measured at a temperature in the range of 60° C. to 90° C. comprising (a) a signal at the following two-theta values: 5.5±0.2, 19.2±0.2, and 19.8±0.2; and (b) a signal at one or more (e.g., two or more) two-theta values chosen from 11.0±0.2, 21.8±0.2, and 27.2±0.2. In some embodiments, the Compound II free form Hemihydrate Form A is characterized by an X-ray powder diffractogram measured at a temperature in the range of 60° C. to 90° C. comprising a signal at one or more two-theta values (e.g., two or more, three or more, four or more, five or more) chosen from 5.5±0.2, 11.0±0.2, 19.2±0.2, 19.8±0.2, 21.8±0.2, and 27.2±0.2. In some embodiments, the Compound II free form Hemihydrate Form A is characterized by an X-ray powder diffractogram measured at a temperature in the range of 60° C. to 90° C. comprising signals at the following two-theta values: 5.5±0.2, 11.0±0.2, 19.2±0.2, 19.8±0.2, 21.8±0.2, and 27.2±0.2.

In some embodiments, the Compound II free form Hemihydrate Form A is characterized by an X-ray powder diffractogram measured at a temperature in the range of 60° C. to 90° C. comprising (a) a signal at the following two-theta values: 5.5±0.2, 19.2±0.2, and 19.8±0.2; and (b) a signal at one or more (e.g., two or more, three or more, four or more) two-theta values chosen from 11.0±0.2, 19.0±0.2, 21.8±0.2, 24.7±0.2, and 27.2±0.2. In some embodiments, the Compound II free form Hemihydrate Form A is characterized by an X-ray powder diffractogram measured at a temperature in the range of 60° C. to 90° C. comprising a signal at one or more two-theta values (e.g., two or more, three or more, four or more, five or more, six or more, seven or more) chosen from 5.5±0.2, 11.0±0.2, 19.0±0.2, 19.2±0.2, 19.8±0.2, 21.8±0.2, 24.7±0.2, and 27.2±0.2. In some embodiments, the Compound II free form Hemihydrate Form A is characterized by an X-ray powder diffractogram measured at a temperature in the range of 60° C. to 90° C. comprising signals at the following two-theta values: 5.5±0.2, 11.0±0.2, 19.0±0.2, 19.2 0.2, 19.8±0.2, 21.8±0.2, 24.7±0.2, and 27.2±0.2.

In some embodiments, the Compound II free form Hemihydrate Form A is characterized by an X-ray powder diffractogram measured at a temperature in the range of 60° C. to 90° C. comprising (a) a signal at the following two-theta values: 5.5±0.2, 19.2±0.2, and 19.8±0.2; and (b) a signal at one or more (e.g., two or more, three or more, four or more, five or more, six or more) two-theta values chosen from 11.0±0.2, 19.0±0.2, 21.8±0.2, 22.0±0.2, 24.3±0.2, 24.7±0.2, and 27.2±0.2. In some embodiments, the Compound II free form Hemihydrate Form A is characterized by an X-ray powder diffractogram measured at a temperature in the range of 60° C. to 90° C. comprising a signal at one or more two-theta values (e.g., two or more, three or more, four or more, five or more, six or more, seven or more, eight or more, nine or more) chosen from 5.5±0.2, 11.0±0.2, 19.0±0.2, 19.2±0.2, 19.8±0.2, 21.8±0.2, 22.0±0.2, 24.3±0.2, 24.7±0.2, and 27.2±0.2. In some embodiments, the Compound II free form Hemihydrate Form A is characterized by an X-ray powder diffractogram measured at a temperature in the range of 60° C. to 90° C. comprising signals at the following two-theta values: 5.5±0.2, 11.0±0.2, 19.0±0.2, 19.2 0.2, 19.8±0.2, 21.8±0.2, 22.0±0.2, 24.3±0.2, 24.7±0.2, and 27.2±0.2.

In some embodiments, the Compound II free form Hemihydrate Form A is characterized by an X-ray powder diffractogram measured at a temperature in the range of 60° C. to 90° C. substantially similar to that in FIG. 30C.

In some embodiments, the Compound II free form Hemihydrate Form A is characterized by a ¹³C NMR spectrum comprising one or more (e.g., two or more, three or more, four or more) signals chosen from 21.9±0.2 ppm, 22.6±0.2 ppm, 67.9±0.2 ppm, 74.6±0.2 ppm, and 139.8±0.2 ppm.

In some embodiments, the Compound II free form Hemihydrate Form A is characterized by a ¹³C NMR spectrum comprising one or more (e.g., two or more, three or more, four or more) signals chosen from 21.9±0.2 ppm, 22.6±0.2 ppm, 133.2±0.2 ppm, 139.8±0.2 ppm, and 140.9±0.2 ppm.

In some embodiments, the Compound II free form Hemihydrate Form A is characterized by a ¹³C NMR spectrum comprising one or more (e.g., two or more, three or more, four or more, five or more, six or more, seven or more, eight or more, nine or more) signals chosen from 21.9±0.2 ppm, 22.6±0.2 ppm, 49.7±0.2 ppm, 65±0.2 ppm, 67.9±0.2 ppm, 74.6±0.2 ppm, 133.2±0.2 ppm, 139.8±0.2 ppm, 140.9±0.2 ppm, and 142.7±0.2 ppm.

In some embodiments, the Compound II free form Hemihydrate Form A is characterized by a ¹³C NMR spectrum comprising one or more (e.g., two or more, three or more, four or more, five or more, six or more, seven or more, eight or more, nine or more) signals chosen from 21.9±0.2 ppm, 22.6±0.2 ppm, 38.4±0.2 ppm, 124.2±0.2 ppm, 124.7±0.2 ppm, 133.2±0.2 ppm, 139.8±0.2 ppm, 140.9±0.2 ppm, 142.7±0.2 ppm, and 147.6±0.2 ppm.

In some embodiments, the Compound II free form Hemihydrate Form A is characterized by a ¹³C NMR spectrum comprising signals at 21.9±0.2 ppm, 22.6±0.2 ppm, 67.9±0.2 ppm, 74.6±0.2 ppm, and 139.8±0.2 ppm.

In some embodiments, the Compound II free form Hemihydrate Form A is characterized by a ¹³C NMR spectrum comprising signals at 21.9±0.2 ppm, 22.6±0.2 ppm, 133.2±0.2 ppm, 139.8±0.2 ppm, and 140.9±0.2 ppm.

In some embodiments, the Compound II free form Hemihydrate Form A is characterized by a ¹³C NMR spectrum comprising signals at 21.9±0.2 ppm, 22.6±0.2 ppm, 49.7±0.2 ppm, 65±0.2 ppm, 67.9±0.2 ppm, 74.6±0.2 ppm, 133.2±0.2 ppm, 139.8±0.2 ppm, 140.9±0.2 ppm, and 142.7±0.2 ppm.

In some embodiments, the Compound II free form Hemihydrate Form A is characterized by a ¹³C NMR spectrum comprising signals at 21.9±0.2 ppm, 22.6±0.2 ppm, 38.4±0.2 ppm, 124.2±0.2 ppm, 124.7±0.2 ppm, 133.2±0.2 ppm, 139.8±0.2 ppm, 140.9±0.2 ppm, 142.7±0.2 ppm, and 147.6±0.2 ppm.

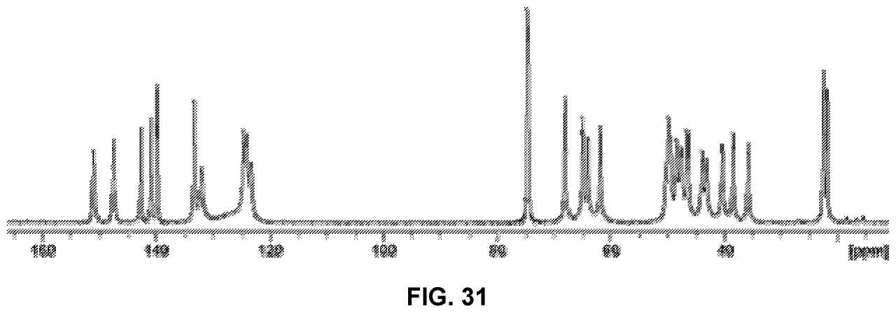

In some embodiments, the Compound II free form Hemihydrate Form A is characterized by a ¹³C NMR spectrum substantially similar to that in FIG. 31.

In some embodiments, the Compound II free form Hemihydrate Form A is characterized by a TGA thermogram showing about 2.4% weight loss from ambient temperature to 150° C.

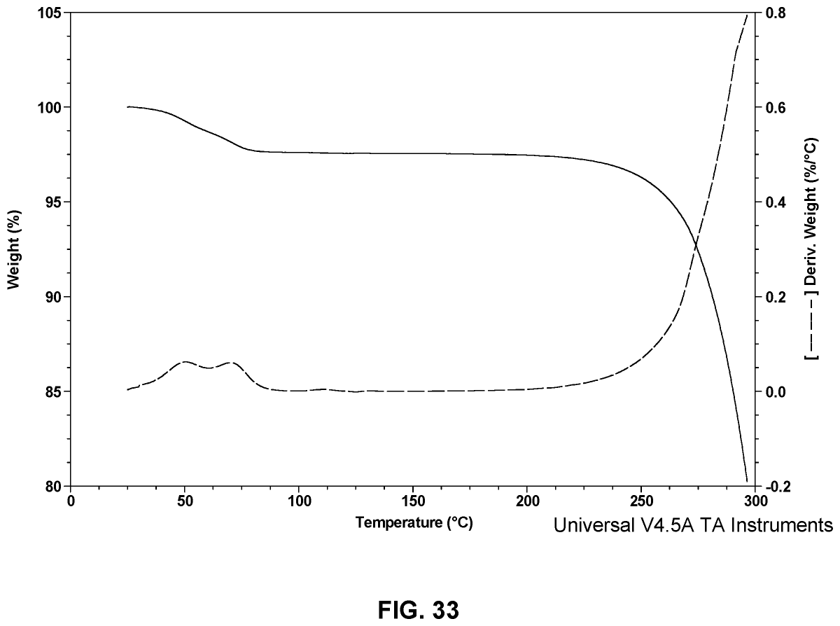

In some embodiments, the Compound II free form Hemihydrate Form A is characterized by a TGA thermogram substantially similar to that in FIG. 33.

In some embodiments, the Compound II free form Hemihydrate Form A is characterized by a DSC curve having endothermic peaks at about 77° C., about 107° C., and about 125° C.

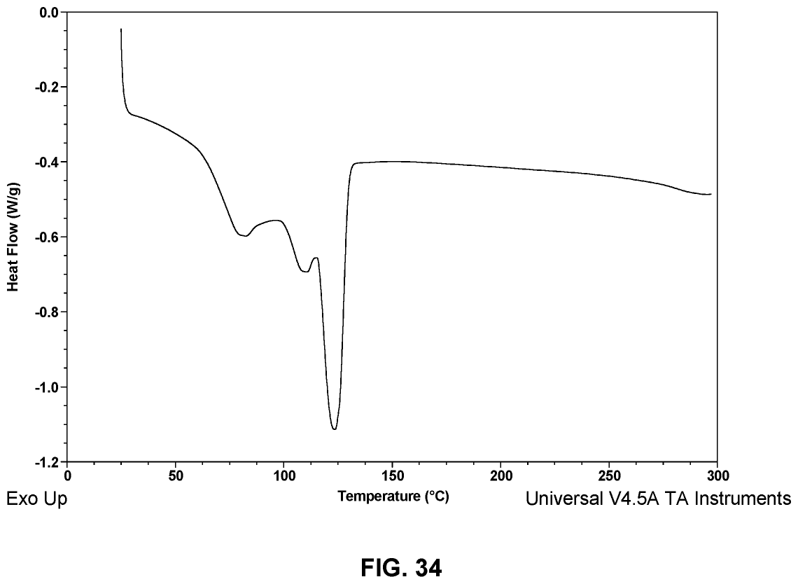

In some embodiments, the Compound II free form Hemihydrate Form A is characterized by a DSC curve substantially similar to that in FIG. 34.

In some embodiments, the Compound II free form Hemihydrate Form A is characterized by a monoclinic crystal system, a P21 space group, and unit cell dimensions measured at 100 K on a Bruker diffractometer equipped Cu Kα radiation (λ=1.54178 Å) of:

| | | | |
|---|---|---|---|
| a | 13.8 ± 0.1 Å | α | 90° |
| b | 8.1 ± 0.1 Å | β | 100.2 ± 0.1° |
| c | 15.6 ± 0.1 Å | γ | 90°. |

Some embodiments of the disclosure provide a method of preparing Compound II free form Hemihydrate Form A comprising:

adding Amorphous free form Compound II to MEK to produce a solution;

adding water and n-Heptane to the solution;

stirring the solution at ambient temperature;

filtering the solution to obtain a solid material; and

drying the solid material.

In some embodiments, stirring the solution at ambient temperature comprises stirring the solution at ambient temperature for about 18 hours. In some embodiments, drying the solid material comprises drying the solid material in a vacuum oven at about 60° C. overnight.

### Compound II Free Form Form C

Some embodiments of the disclosure provide a free form of Compound II (Compound II free form Form C). In some embodiments, the Compound II free form Form C is substantially pure.

In some embodiments, the Compound II free form Form C is characterized by an X-ray powder diffractogram comprising a signal at 11.1±0.2 two-theta. In some embodiments, the Compound II free form Form C is characterized by an X-ray powder diffractogram comprising a signal at 13.0±0.2 two-theta. In some embodiments, the Compound II free form Form C is characterized by an X-ray powder diffractogram comprising a signal at 19.8±0.2 two-theta. In some embodiments, the Compound II free form Form C is characterized by an X-ray powder diffractogram comprising a signal at 21.6±0.2 two-theta.

In some embodiments, the Compound II free form Form C is characterized by an X-ray powder diffractogram comprising a signal at two or more two-theta values chosen from 11.1±0.2, 13.0±0.2, 19.8±0.2, and 21.6±0.2. In some embodiments, the Compound II free form Form C is characterized by an X-ray powder diffractogram comprising a signal at three or more two-theta values chosen from 11.1±0.2, 13.0±0.2, 19.8±0.2, and 21.6±0.2. In some embodiments, the Compound II free form Form C is characterized by an X-ray powder diffractogram comprising signals 11.1±0.2 two-theta, 13.0±0.2 two-theta, 19.8±0.2 two-theta, and 21.6±0.2 two-theta.

In some embodiments, the Compound II free form Form C is characterized by an X-ray powder diffractogram comprising (a) a signal at one or more two-theta values chosen from 11.1±0.2, 13.0±0.2, 19.8±0.2, and 21.6±0.2; and (b) a signal at one or more two-theta values chosen from 15.7±0.2, 17.7±0.2, 18.5±0.2 and 23.6±0.2. In some embodiments, the Compound II free form Form C is characterized by an X-ray powder diffractogram comprising (a) a signal at one or more two-theta values chosen from 11.1±0.2, 13.0±0.2, 19.8±0.2, and 21.6±0.2; and (b) a signal at two or more two-theta values chosen from 15.7±0.2, 17.7±0.2, 18.5±0.2 and 23.6±0.2. In some embodiments, the Compound II free form Form C is characterized by an X-ray powder diffractogram comprising (a) a signal at one or more two-theta values chosen from 11.1±0.2, 13.0±0.2, 19.8±0.2, and 21.6±0.2; and (b) a signal at three or more two-theta values chosen from 15.7±0.2, 17.7±0.2, 18.5±0.2 and 23.6±0.2. In some embodiments, the Compound II free form Form C is characterized by an X-ray powder diffractogram comprising (a) a signal at one or more two-theta values chosen from 11.1±0.2, 13.0±0.2, 19.8±0.2, and 21.6±0.2; and (b) signals at 15.7±0.2 two-theta, 17.7±0.2 two-theta, 18.5±0.2 two-theta and 23.6±0.2 two-theta.

In some embodiments, the Compound II free form Form C is characterized by an X-ray powder diffractogram comprising (a) a signal at two or more two-theta values chosen from 11.1±0.2, 13.0±0.2, 19.8±0.2, and 21.6±0.2; and (b) signals at 15.7±0.2 two-theta, 17.7±0.2 two-theta, 18.5±0.2 two-theta and 23.6±0.2 two-theta. In some embodiments, the Compound II free form Form C is characterized by an X-ray powder diffractogram comprising (a) a signal at three or more two-theta values chosen from 11.1±0.2, 13.0±0.2, 19.8±0.2, and 21.6±0.2; and (b) signals at 15.7±0.2 two-theta, 17.7±0.2 two-theta, 18.5±0.2 two-theta and 23.6±0.2 two-theta. In some embodiments, the Compound II free form Form C is characterized by an X-ray powder diffractogram comprising signals 11.1±0.2 two-theta, 13.0±0.2 two-theta, 19.8±0.2 two-theta, 21.6±0.2 two-theta, 15.7±0.2 two-theta, 17.7±0.2 two-theta, 18.5±0.2 two-theta and 23.6±0.2 two-theta.

In some embodiments, the Compound II free form Form C is characterized by an X-ray powder diffractogram comprising (a) a signal at two or more of the following two-theta values: 11.1±0.2, 13.0±0.2, 19.8±0.2, and 21.6±0.2; and (b) a signal at one or more two-theta (e.g., two or more, three or more, four or more, five or more, etc.) values chosen from 11.1±0.2, 15.5±0.2, and 15.7±0.2, 16.5±0.2, 17.1±0.2, 17.7±0.2, 17.9±0.2, 18.5±0.2, 22.0±0.2, 23.3±0.2, 23.6±0.2, 24.0±0.2, 26.3±0.2, 26.7±0.2, 26.8±0.2, 30.6±0.2. In some embodiments, the Compound II free form Form C is characterized by an X-ray powder diffractogram comprising (a) a signal at three or more of the following two-theta values: 11.1±0.2, 13.0±0.2, 19.8±0.2, and 21.6±0.2; and (b) a signal at one or more two-theta (e.g., two or more, three or more, four or more, five or more, etc.) values chosen from 11.1±0.2, 15.5±0.2, and 15.7±0.2, 16.5±0.2, 17.1±0.2, 17.7±0.2, 17.9±0.2, 18.5±0.2, 22.0±0.2, 23.3±0.2, 23.6±0.2, 24.0±0.2, 26.3±0.2, 26.7±0.2, 26.8±0.2, 30.6±0.2. In some embodiments, the Compound II free form Form C is characterized by an X-ray powder diffractogram comprising (a) a signal at each of two-theta values 11.1±0.2, 13.0±0.2, 19.8±0.2 and 21.6±0.2; and (b) a signal at one or more two-theta values (e.g., two or more, three or more, four or more, five or more) chosen from 15.5±0.2, 15.7±0.2, 16.5±0.2, 17.1±0.2, 17.7±0.2, 17.9±0.2, 18.5±0.2, 22.0±0.2, 23.3±0.2, 23.6±0.2, 24.0±0.2, 26.3±0.2, 26.7±0.2, 26.8±0.2, 30.6±0.2.

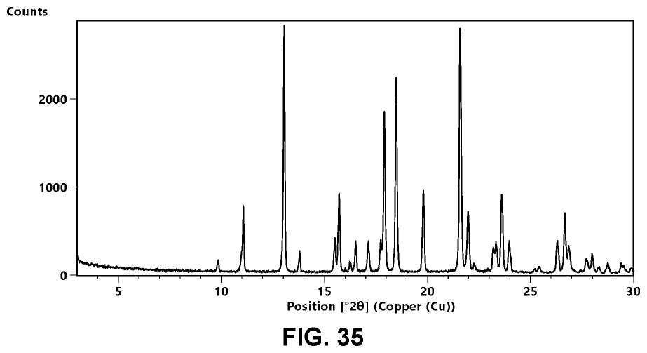

In some embodiments, the Compound II free form Form C is characterized by an X-ray powder diffractogram substantially similar to that in FIG. 35.

In some embodiments, the Compound II free form Form C is characterized by a TGA thermogram showing negligible weight loss from ambient temperature up to 200° C.

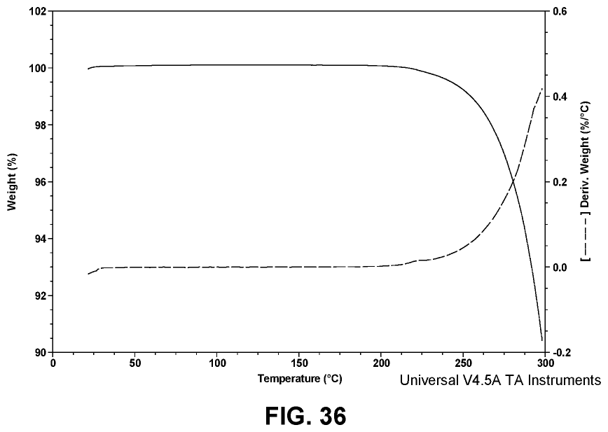

In some embodiments, the Compound II free form Form C is characterized by a TGA thermogram substantially similar to that in FIG. 36.

In some embodiments, the Compound II free form Form C is characterized by a DSC curve having an endothermic peak at about 218° C.

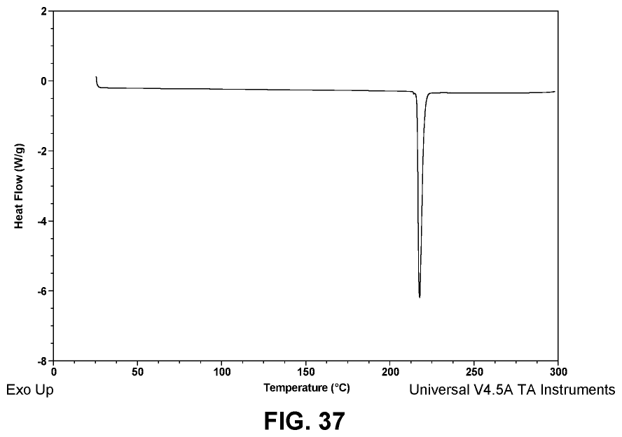

In some embodiments, the Compound II free form Form C is characterized by a DSC curve substantially similar to that in FIG. 37.

In some embodiments, the Compound II free form Form C is characterized by a ¹³C NMR spectrum comprising one or more (e.g., two or more, three or more, four or more, etc.) signals chosen from 149.3±0.2 ppm, 144.3±0.2 ppm, 135.0±0.2 ppm, 127.2±0.2 ppm, and 124.5±0.2 ppm. In some embodiments, the Compound II free form Form C is characterized by a ¹³C NMR spectrum comprising signals at 149.3±0.2 ppm, 144.3±0.2 ppm, 135.0±0.2 ppm, 127.2±0.2 ppm, and 124.5±0.2 ppm.

In some embodiments, the Compound II free form Form C is characterized by a ¹³C NMR spectrum comprising one or more signals chosen from 66.9±0.2 ppm, 49.4±0.2 ppm, 37.7±0.2 ppm, 36.8±0.2 ppm, and 25.9±0.2 ppm. In some embodiments, the Compound II free form Form C is characterized by a ¹³C NMR spectrum comprising two or more signals chosen from 66.9±0.2 ppm, 49.4±0.2 ppm, 37.7±0.2 ppm, 36.8±0.2 ppm, and 25.9±0.2 ppm. In some embodiments, the Compound II free form Form C is characterized by a ¹³C NMR spectrum comprising three or more signals chosen from 66.9±0.2 ppm, 49.4±0.2 ppm, 37.7±0.2 ppm, 36.8±0.2 ppm, and 25.9±0.2 ppm. In some embodiments, the Compound II free form Form C is characterized by a ¹³C NMR spectrum comprising four or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm. In some embodiments, the Compound II free form Form C is characterized by a ¹³C NMR spectrum comprising five or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm. In some embodiments, the Compound II free form Form C is characterized by a ¹³C NMR spectrum comprising six or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm. In some embodiments, the Compound II free form Form C is characterized by a ¹³C NMR spectrum comprising seven or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm. In some embodiments, the Compound II free form Form C is characterized by a ¹³C NMR spectrum comprising signals at 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.

In some embodiments, the Compound II free form Form C is characterized by a ¹³C NMR spectrum comprising (a) one or more (e.g., two or more, three or more, four or more, etc.) signals chosen from 149.3±0.2 ppm, 144.3±0.2 ppm, 135.0±0.2 ppm, 127.2±0.2 ppm, and 124.5±0.2 ppm; and (b) one or more (e.g., two or more, three or more, four or more, etc.) signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.

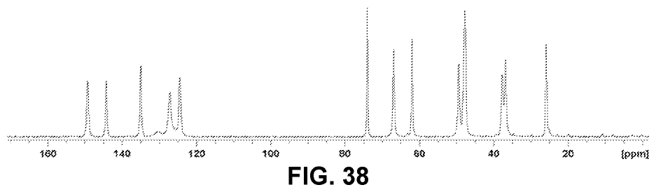

In some embodiments, the Compound II free form Form C is characterized by a ¹³C NMR spectrum substantially similar to that in FIG. 38.

In some embodiments, the Compound II free form Form C is characterized by a single crystal unit cell characterized by an orthorhombic crystal system, a P2₁2₁2₁ space group, and unit cell dimensions measured at 298 K on a Bruker diffractometer equipped Cu Kα radiation (λ=1.54178 Å) of:

| | | | |
|---|---|---|---|
| a | 10.3 ± 0.1 Å | α | 90° |
| b | 12.5 ± 0.1 Å | β | 90° |
| c | 12.8 ± 0.1 Å | γ | 90°. |

In some embodiments, the Compound II free form Form C is characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 10.3 ± 0.1 Å | α | 90° |
| b | 12.3 ± 0.1 Å | β | 90° |
| c | 12.7 ± 0.1 Å | γ | 90°. |

Some embodiments of the disclosure provide a method of preparing Compound II free form Form C comprising:

adding 0.5 ml MEK to Compound II free form Hemihydrate Form A;

stirring at 20° C. overnight; and

isolating the solids.

### Compound II Free Form Form A

Some embodiments of the disclosure provide a free form of Compound II (Compound II free form Form A). In some embodiments, the Compound II free form Form A is substantially pure.

In some embodiments, the Compound II free form Form A is characterized by an X-ray powder diffractogram comprising a signal at 9.1±0.2 two-theta. In some embodiments, the Compound II free form Form A is characterized by an X-ray powder diffractogram comprising a signal at 11.7±0.2 two-theta. In some embodiments, the Compound II free form Form A is characterized by an X-ray powder diffractogram comprising a signal at 13.9±0.2 two-theta. In some embodiments, the Compound II free form Form A is characterized by an X-ray powder diffractogram comprising a signal at 14.1±0.2 two-theta. In some embodiments, the Compound II free form Form A is characterized by an X-ray powder diffractogram comprising a signal at 20.5±0.2 two-theta.

In some embodiments, the Compound II free form Form A is characterized by an X-ray powder diffractogram comprising a signal at two or more two-theta values chosen from 9.1±0.2, 11.7±0.2, 13.9±0.2, 14.1±0.2, and 20.5±0.2. In some embodiments, the Compound II free form Form A is characterized by an X-ray powder diffractogram comprising a signal at three or more two-theta values chosen from 9.1±0.2, 11.7±0.2, 13.9±0.2, 14.1±0.2, and 20.5±0.2. In some embodiments, the Compound II free form Form A is characterized by an X-ray powder diffractogram comprising a signal at four or more two-theta values chosen from 9.1±0.2, 11.7±0.2, 13.9±0.2, 14.1±0.2, and 20.5±0.2. In some embodiments, the Compound II free form Form A is characterized by an X-ray powder diffractogram comprising signals at 9.1±0.2 two-theta, 11.7±0.2 two-theta, 13.9±0.2 two-theta, 14.1±0.2 two-theta, and 20.5±0.2 two-theta.

In some embodiments, the Compound II free form Form A is characterized by an X-ray powder diffractogram comprising (a) a signal at one or more two-theta values chosen from 9.1±0.2, 11.7±0.2, 13.9±0.2, 14.1±0.2, and 20.5±0.2; and (b) a signal at one or more (e.g., two, three, four, or five) two-theta values chosen from 16.6±0.2, 17.3±0.2, 18.3±0.2, 22.1±0.2, and 24.4±0.2. In some embodiments, the Compound II free form Form A is characterized by an X-ray powder diffractogram comprising (a) a signal at two or more two-theta values chosen from 9.1±0.2, 11.7±0.2, 13.9±0.2, 14.1±0.2, and 20.5±0.2; and (b) a signal at one or more (e.g., two, three, four, or five) two-theta values chosen from 16.6±0.2, 17.3±0.2, 18.3±0.2, 22.1±0.2, and 24.4±0.2. In some embodiments, the Compound II free form Form A is characterized by an X-ray powder diffractogram comprising (a) a signal at three or more two-theta values chosen from 9.1±0.2, 11.7±0.2, 13.9±0.2, 14.1±0.2, and 20.5±0.2; and (b) a signal at one or more (e.g., two, three, four, or five) two-theta values chosen from 16.6±0.2, 17.3±0.2, 18.3±0.2, 22.1±0.2, and 24.4±0.2. In some embodiments, the Compound II free form Form A is characterized by an X-ray powder diffractogram comprising (a) signals at 9.1±0.2 two-theta, 11.7±0.2 two-theta, 13.9±0.2 two-theta, 14.1±0.2 two-theta, and 20.5±0.2 two-theta; and (b) a signal at one or more (e.g., two, three, four, or five) two-theta values chosen from 16.6±0.2, 17.3±0.2, 18.3±0.2, 22.1±0.2, and 24.4±0.2.

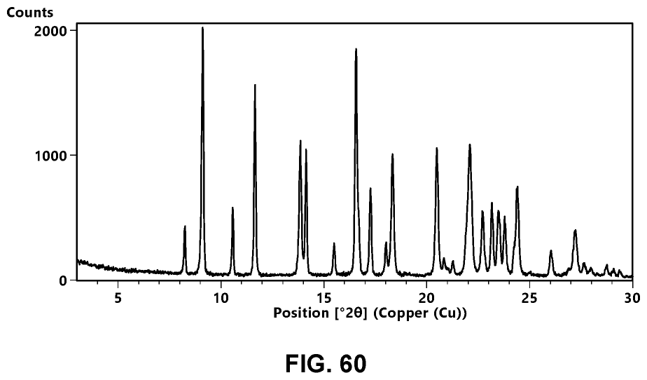

In some embodiments, the Compound II free form Form A is characterized by an X-ray powder diffractogram substantially similar to that in FIG. 60.

In some embodiments, the Compound II free form Form A is characterized by a TGA thermogram showing negligible weight loss from ambient temperature up to 200° C.

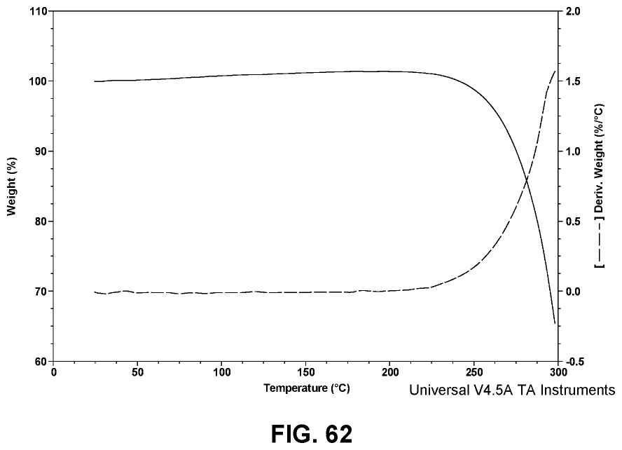

In some embodiments, the Compound II free form Form A is characterized by a TGA thermogram substantially similar to that in FIG. 62.

In some embodiments, the Compound II free form Form A is characterized by a DSC curve having an endothermic peak at about 130° C.

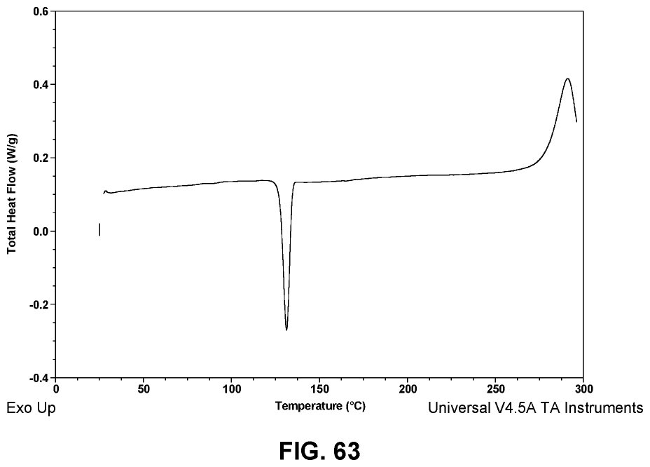

In some embodiments, the Compound II free form Form A is characterized by a DSC curve substantially similar to that in FIG. 63.

In some embodiments, the Compound II free form Form A is characterized by a ¹³C NMR spectrum comprising one or more (e.g., two or more, three or more, four) signals chosen from 143.6±0.2 ppm, 134.1±0.2 ppm, 128.8±0.2 ppm, and 123.4±0.2 ppm. In some embodiments, the Compound II free form Form A is characterized by a ¹³C NMR spectrum comprising signals at 143.6±0.2 ppm, 134.1±0.2 ppm, 128.8±0.2 ppm, and 123.4±0.2 ppm.

In some embodiments, the Compound II free form Form A is characterized by a ¹³C NMR spectrum comprising one or more (e.g., two or more, three or more, four or more, five) signals chosen from 68.3±0.2 ppm, 48.9±0.2 ppm, 39.1±0.2 ppm, and 21.6±0.2 ppm. In some embodiments, the Compound II free form Form A is characterized by a ¹³C NMR spectrum comprising signals at 68.3±0.2 ppm, 48.9±0.2 ppm, 39.6±0.2 ppm, 39.1±0.2 ppm, and 21.6±0.2 ppm.

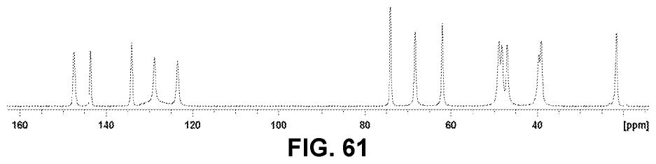

In some embodiments, the Compound II free form Form A is characterized by a ¹³C NMR spectrum substantially similar to that in FIG. 61.

In some embodiments, the Compound II free form Form A is characterized by a single crystal unit cell characterized by a monoclinic crystal system, a 12 space group, and unit cell dimensions measured at 298 K on a Bruker diffractometer equipped Cu Kα radiation (λ=1.54178 Å) of:

| | | | |
|---|---|---|---|
| a | 10.1 ± 0.1 Å | α | 90° |
| b | 8.0 ± 0.1 Å | β | 101.0 ± 0.1° |
| c | 21.8 ± 0.1 Å | γ | 90°. |

Some embodiments of the disclosure provide a method of preparing Compound II Form A comprising:

desolvating Compound II free form MeOH Solvate in a 40° C. vacuum oven; and

isolating the solids.

### Compound II Free Form Form B

Some embodiments of the disclosure provide a free form of Compound II (Compound II free form Form B). In some embodiments, the Compound II free form Form B is substantially pure.

In some embodiments, the Compound II free form Form B is characterized by a ¹³C NMR spectrum comprising one or more (e.g., two or more, three or more, four or more, five) signals chosen from 22.4±0.2 ppm, 22.6±0.2 ppm, 47.7±0.2 ppm, 64.1±0.2 ppm, and 74.6±0.2 ppm.

In some embodiments, the Compound II free form Form B is characterized by a ¹³C NMR spectrum comprising one or more (e.g., two or more, three or more, four or more, five) signals chosen from 22.4±0.2 ppm, 22.6±0.2 ppm, 38.5±0.2 ppm, 132.9±0.2 ppm, and 139.4±0.2 ppm.

In some embodiments, the Compound II free form Form B is characterized by a ¹³C NMR spectrum measured comprising one or more (e.g., two or more, three or more, four or more, five or more, six or more, seven or more, eight or more, nine or more) signals chosen from 22.4±0.2 ppm, 22.6±0.2 ppm, 44.3±0.2 ppm, 47.3±0.2 ppm, 47.7±0.2 ppm, 61.8±0.2 ppm, 64.1±0.2 ppm, 67.6±0.2 ppm, 74.6±0.2 ppm, and 139.4±0.2 ppm.

In some embodiments, the Compound II free form Form B is characterized by a ¹³C NMR spectrum comprising one or more (e.g., two or more, three or more, four or more, five or more, six or more, seven or more, eight or more, nine or more, ten) signals chosen from 22.4±0.2 ppm, 22.6±0.2 ppm, 35.3±0.2 ppm, 38.5±0.2 ppm, 39.8±0.2 ppm, 124.4±0.2 ppm, 132.9±0.2 ppm, 139.4±0.2 ppm, 141.5±0.2 ppm, and 142.2±0.2 ppm.

In some embodiments, the Compound II free form Form B is characterized by a ¹³C NMR spectrum comprising signals at 22.4±0.2 ppm, 22.6±0.2 ppm, 47.7±0.2 ppm, 64.1±0.2 ppm, and 74.6±0.2 ppm.

In some embodiments, the Compound II free form Form B is characterized by a ¹³C NMR spectrum comprising signals at 22.4±0.2 ppm, 22.6±0.2 ppm, 38.5±0.2 ppm, 132.9±0.2 ppm, and 139.4±0.2 ppm.

In some embodiments, the Compound II free form Form B is characterized by a ¹³C NMR spectrum comprising signals at 22.4±0.2 ppm, 22.6±0.2 ppm, 44.3±0.2 ppm, 47.3±0.2 ppm, 47.7±0.2 ppm, 61.8±0.2 ppm, 64.1±0.2 ppm, 67.6±0.2 ppm, 74.6±0.2 ppm, and 139.4±0.2 ppm.

In some embodiments, the Compound II free form Form B is characterized by a ¹³C NMR spectrum comprising signals at 22.4±0.2 ppm, 22.6±0.2 ppm, 35.3±0.2 ppm, 38.5±0.2 ppm, 39.8±0.2 ppm, 124.4±0.2 ppm, 132.9±0.2 ppm, 139.4±0.2 ppm, 141.5±0.2 ppm, and 142.2±0.2 ppm.

In some embodiments, the Compound II free form Form B is characterized by a ¹³C NMR spectrum comprising one or more (e.g., two or more, three or more, four or more, five) signals chosen from 22.4±0.2 ppm, 22.6±0.2 ppm, 44.3±0.2 ppm, 47.3±0.2 ppm, 64.1±0.2 ppm, 67.6±0.2 ppm, 74.6±0.2 ppm, 132.9±0.2 ppm, 139.4±0.2 ppm.

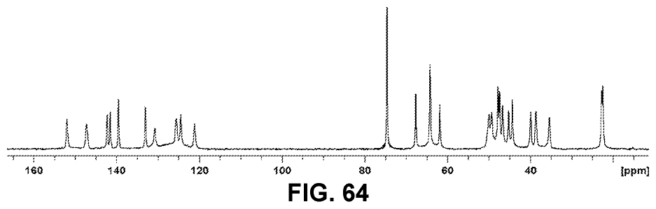

In some embodiments, the Compound II free form Form B is characterized by a 3C NMR spectrum substantially similar to that in FIG. 64.

In some embodiments, the Compound II free form Form B is characterized by a single crystal unit cell characterized by a monoclinic crystal system, a P2₁ space group, and unit cell dimensions measured at 100 K on a Bruker diffractometer equipped Cu Kα radiation (λ=1.54178 Å) of:

| | | | |
|---|---|---|---|
| a | 13.4 ± 0.1 Å | α | 90° |
| b | 8.1 ± 0.1 Å | β | 101.1 ± 0.1° |
| c | 16.0 ± 0.1 Å | γ | 90°. |

Some embodiments of the disclosure provide a method of preparing Compound II Form B comprising:

loading Compound II free form Hemihydrate Form A into an ssNMR rotor;

drying overnight in an 80° C. oven; and

sealing with rotor cap before removing solid from oven for analysis.

### Compound II Free Form Quarter Hydrate

Some embodiments of the disclosure provide a free form of Compound II (Compound II free form Quarter Hydrate). In some embodiments, the Compound II free form Quarter Hydrate is substantially pure.

In some embodiments, the Compound II free form Quarter Hydrate is characterized by a ¹³C NMR spectrum comprising a signal at 64.5±0.2 ppm. In some embodiments, the Compound II free form Quarter Hydrate is characterized by a ¹³C NMR spectrum comprising one or more (e.g., two, three, or four) signals chosen from 151.8±0.2 ppm, 151.5±0.2 ppm, 121.1±0.2 ppm, and 35.3±0.2 ppm. In some embodiments, the Compound II free form Quarter Hydrate is characterized by a ¹³C NMR spectrum comprising signals at 151.8±0.2 ppm, 151.5±0.2 ppm, 121.1±0.2 ppm, 64.5±0.2 ppm, and 35.3±0.2 ppm.

In some embodiments, the Compound II free form Quarter Hydrate is characterized by a ¹³C NMR spectrum comprising (a) one or more (e.g., two, three, or more, four) signals chosen from 151.8±0.2 ppm, 151.5±0.2 ppm, ppm, 121.1±0.2 ppm, and 35.3±0.2 ppm; and (b) one or more (e.g., two or more, three or more, four or more, five or more, six or more, seven or more, eight) signals at 74.4±0.2 ppm, 67.6±0.2 ppm, 64.5±0.2 ppm, 61.8±0.2 ppm, 47.5±0.2 ppm, 47.2±0.2 ppm, 44.1±0.2 ppm, and 22.1±0.2 ppm. In some embodiments, the Compound II free form Quarter Hydrate is characterized by a ¹³C NMR spectrum comprising (a) a signal at 64.5±0.2 ppm; and (b) one or more (e.g., two or more, three or more, four or more, five or more, six or more, seven) signals at 74.4±0.2 ppm, 67.6±0.2 ppm, 61.8±0.2 ppm, 47.5±0.2 ppm, 47.2±0.2 ppm, 44.1±0.2 ppm, and 22.1±0.2 ppm.

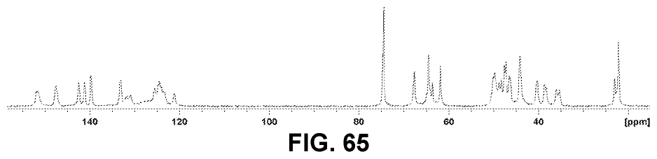

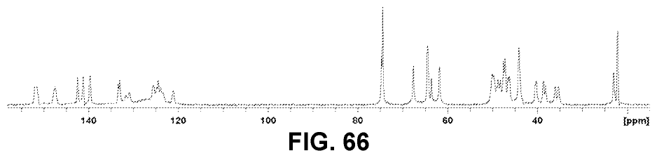

In some embodiments, the Compound II free form Quarter Hydrate is characterized by a ¹³C NMR spectrum substantially similar to that in FIG. 65 or FIG. 66.

In some embodiments, the Compound II free form Quarter Hydrate is characterized by a single crystal unit cell characterized by a monoclinic crystal system, a P21 space group, and unit cell dimensions measured at 100 K on a Bruker diffractometer equipped Cu Kα radiation (λ=1.54178 Å) of:

| | | | |
|---|---|---|---|
| a | 18.9 ± 0.1 Å | α | 90° |
| b | 8.1 ± 0.1 Å | β | 99.1 ± 0.1° |
| c | 22.6 ± 0.1 Å | γ | 90°. |

Some embodiments of the disclosure provide a method of preparing Compound II free form Quarter Hydrate comprising:

dehydrating Compound II free form Hemihydrate Form A in isothermal 80° C. TGA for 1 hour;

unloading the solid to pack in the rotor as quickly as possible; and

sealing with rotor cap as soon as the solid is loaded for analysis.

### Compound II Free Form Hydrate Mixture

In some embodiments, the Compound II free form Hydrate Mixture is characterized by an X-ray powder diffractogram comprising a signal at 8.6±0.2 two-theta. In some embodiments, the Compound II free form Hydrate Mixture is characterized by an X-ray powder diffractogram comprising a signal at 24.1±0.2 two-theta. In some embodiments, the Compound II free form Hydrate Mixture is characterized by an X-ray powder diffractogram comprising a signal at 24.5±0.2 two-theta. In some embodiments, the Compound II free form Hydrate Mixture is characterized by an X-ray powder diffractogram comprising a signal at 13.7±0.2 two-theta. In some embodiments, the Compound II free form Hydrate Mixture is characterized by an X-ray powder diffractogram comprising a signal at 3.6±0.2 two-theta. In some embodiments, the Compound II free form Hydrate Mixture is characterized by an X-ray powder diffractogram comprising a signal at 19.9±0.2 two-theta.

In some embodiments, the Compound II free form Hydrate Mixture is characterized by an X-ray powder diffractogram comprising a signal at one or more (e.g., two or more, three or more, four or more, five or more, six) two-theta values chosen from 3.6±0.2, 8.6±0.2, 13.7±0.2, 19.9±0.2, 24.1±0.2, and 24.5±0.2. In some embodiments, the Compound II free form Hydrate Mixture is characterized by an X-ray powder diffractogram comprising signals at 3.6±0.2 two-theta, 8.6±0.2 two-theta, 13.7±0.2 two-theta, 19.9±0.2 two-theta, 24.1±0.2 two-theta, and 24.5±0.2 two-theta.

In some embodiments, the Compound II free form Hydrate Mixture is characterized by an X-ray powder diffractogram comprising (a) a signal at one or more (e.g., two or more, three or more, four or more, five or more, six) two-theta values chosen from 3.6±0.2, 8.6±0.2, 13.7±0.2, 19.9±0.2, 24.1±0.2, and 24.5±0.2; and (b) a signal at one or more (e.g., two or more, three or more, four) two-theta values chosen from 22.2±0.2, 21.6±0.2, 17.0±0.2, and 14.6±0.2. In some embodiments, the Compound II free form Hydrate Mixture is characterized by an X-ray powder diffractogram comprising (a) a signal at one or more (e.g., two or more, three or more, four or more, five or more, six) two-theta values chosen from 3.6±0.2, 8.6±0.2, 13.7±0.2, 19.9±0.2, 24.1±0.2, and 24.5±0.2; and (b) signals at 22.2±0.2 two-theta, 21.6±0.2 two-theta, 17.0±0.2 two-theta, and 14.6±0.2 two-theta.

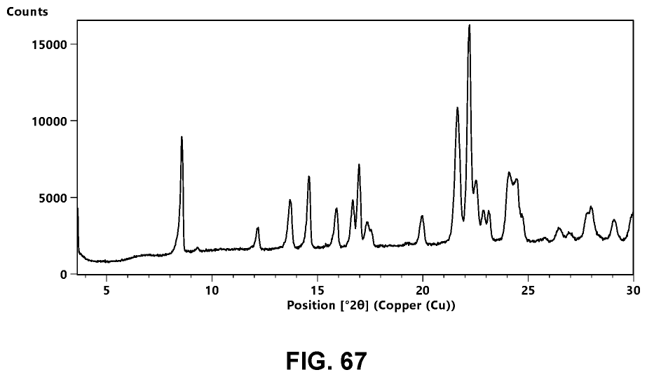

In some embodiments, the Compound II free form Hydrate Mixture is characterized by an X-ray powder diffractogram substantially similar to that in FIG. 67.

Some embodiments of the disclosure provide a method of preparing Compound II free form Hydrate Mixture comprising:

equilibrating Compound II Neat Form A for 3 days in a humidified chamber set at 95% RH; and

isolating the solids.

### Compound II Free Form Monohydrate

In some embodiments, the Compound II free form Monohydrate is characterized by a ¹³C NMR spectrum comprising a signal at 134.1±0.2 ppm. In some embodiments, the Compound II free form Monohydrate is characterized by a ¹³C NMR spectrum comprising a signal at 21.1±0.2 ppm. In some embodiments, the Compound II free form Monohydrate is characterized by a ¹³C NMR spectrum comprising a signal at 134.1±0.2 ppm and a signal at 21.1±0.2 ppm.

In some embodiments, the Compound II free form Monohydrate is characterized by a ¹³C NMR spectrum comprising (a) a signal at 134.1±0.2 ppm and/or a signal at 21.1±0.2 ppm; and (b) one or more signals (e.g., two, three, four, or five) signals chosen from 74.5±0.2 ppm, 62.4±0.2 ppm, 49.0±0.2 ppm, 39.1±0.2 ppm, and 21.7±0.2 ppm. In some embodiments, the Compound II free form Monohydrate is characterized by a ¹³C NMR spectrum comprising (a) a signal at 134.1±0.2 ppm and/or a signal at 21.1±0.2 ppm; and (b) signals at 74.5±0.2 ppm, 62.4±0.2 ppm, 49.0±0.2 ppm, 39.1±0.2 ppm, and 21.7±0.2 ppm.

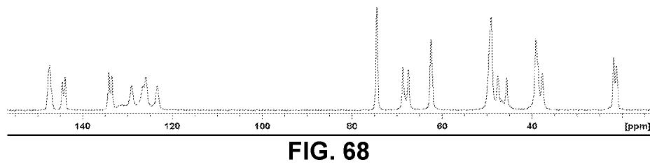

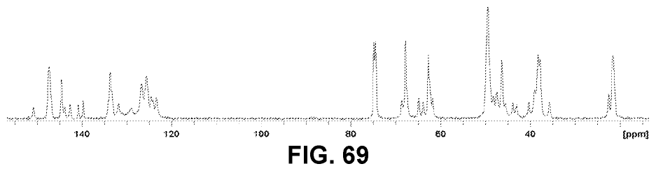

In some embodiments, the Compound II free form Monohydrate is characterized by a ¹³C NMR spectrum substantially similar to that in FIG. 68.

Some embodiments of the disclosure provide a method of preparing Compound II free form Monohydrate comprising:

humidifying Compound II Neat Form A in 69% RH chamber equilibrating in saturated potassium iodide for 1-2 months under static conditions; and

isolating the solid.

### Compound II Free Form Dihydrate

In some embodiments, the Compound II free form Dihydrate is characterized by a ¹³C NMR spectrum comprising a signal at 143.8±0.2 ppm and a signal at 38.2±0.2 ppm. In some embodiments, the Compound II free form Dihydrate is characterized by a ¹³C NMR spectrum comprising (a) one or more (e.g., two, three. four, five, or six) signals chosen from 143.8±0.2 ppm, 128.9±0.2 ppm, 126.6±0.2 ppm, 68.6±0.2 ppm, 62.7±0.2 ppm, and 37.8±0.2 ppm; and (b) one or more (e.g., two, three. four, or five) signals chosen from 131.8±0.2 ppm, 124.5±0.2 ppm, 124.1±0.2 ppm, 38.2±0.2 ppm, and 22.5±0.2 ppm.

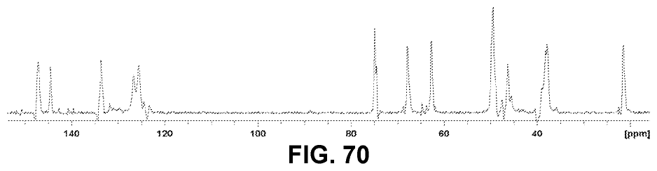

In some embodiments, the Compound II free form Dihydrate is characterized by a ¹³C NMR spectrum substantially similar to that in FIG. 69 or FIG. 70.

Some embodiments of the disclosure provide a method of preparing Compound II 94% RH Hydrate comprising:

humidifying Compound II Neat Form A in 94% RH chamber equilibrating in saturated potassium nitrate for 12 days under static conditions; and

isolating the solid.

### Compound II Free Form EtOH Solvate Form B

Some embodiments of the disclosure provide an EtOH solvate form of Compound II (Compound II free form EtOH Solvate Form B). In some embodiments, the Compound II free form EtOH Solvate Form B is substantially pure.

In some embodiments, the Compound II free form EtOH Solvate Form B is characterized by an X-ray powder diffractogram comprising a signal at 11.6±0.2 two-theta. In some embodiments, the Compound II free form EtOH Solvate Form B is characterized by an X-ray powder diffractogram comprising a signal at 17.1±0.2 two-theta. In some embodiments, the Compound II free form EtOH Solvate Form B is characterized by an X-ray powder diffractogram comprising a signal at 23.8±0.2 two-theta.

In some embodiments, the Compound II free form EtOH Solvate Form B is characterized by an X-ray powder diffractogram comprising a signal at 11.6±0.2 two-theta and a signal at 17.1±0.2 two-theta. In some embodiments, the Compound II free form EtOH Solvate Form B is characterized by an X-ray powder diffractogram comprising a signal at 11.6±0.2 two-theta and a signal at 23.8±0.2 two-theta. In some embodiments, the Compound II free form EtOH Solvate Form B is characterized by an X-ray powder diffractogram comprising a signal at 17.1±0.2 two-theta and a signal at 23.8±0.2 two-theta. In some embodiments, the Compound II free form EtOH Solvate Form B is characterized by an X-ray powder diffractogram comprising a signal at 11.6±0.2 two-theta, a signal at 17.1±0.2 two-theta, and a signal at 23.8±0.2 two-theta.

In some embodiments, the Compound II free form EtOH Solvate Form B is characterized by an X-ray powder diffractogram comprising (a) a signal at one or more two-theta values chosen from 11.6±0.2, 17.1±0.2, and 23.8±0.2; and (b) a signal at one or more (e.g., two, three, or four) two-theta values chosen from 7.6±0.2, 16.6±0.2, 23.3±0.2 and 23.7±0.2. In some embodiments, the Compound II free form EtOH Solvate Form B is characterized by an X-ray powder diffractogram comprising (a) a signal at two or more two-theta values chosen from 11.6±0.2, 17.1±0.2, and 23.8±0.2; and (b) a signal at one or more (e.g., two, three, or four) two-theta values chosen from 7.6±0.2, 16.6±0.2, 23.3±0.2 and 23.7±0.2. In some embodiments, the Compound II free form EtOH Solvate Form B is characterized by an X-ray powder diffractogram comprising (a) a signal 11.6±0.2 two-theta, 17.1±0.2 two-theta, and 23.8±0.2 two-theta; and (b) a signal at one or more (e.g., two, three, or four) two-theta values chosen from 7.6±0.2, 16.6±0.2, 23.3±0.2 and 23.7±0.2.

In some embodiments, the Compound II free form EtOH Solvate Form B is characterized by an X-ray powder diffractogram comprising a signal 7.6±0.2 two-theta, 11.6±0.2 two-theta, 16.6±0.2 two-theta, 17.1±0.2 two-theta, 23.3±0.2 two-theta, 23.7±0.2 two-theta, and 23.8±0.2 two-theta.

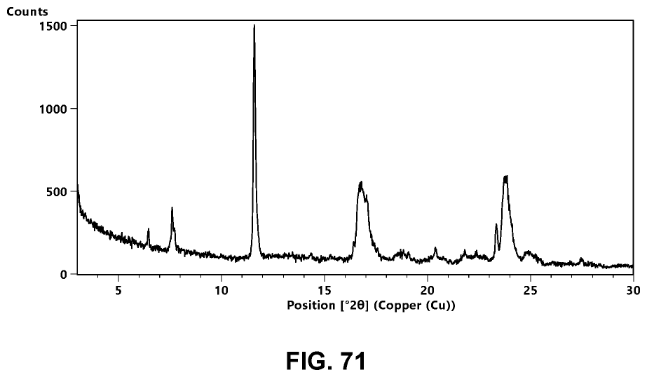

In some embodiments, the Compound II free form EtOH Solvate Form B is characterized by an X-ray powder diffractogram substantially similar to that in FIG. 71.

In some embodiments, the Compound II free form EtOH Solvate Form B is characterized by a TGA thermogram showing about 9% weight loss from ambient temperature up to 200° C.

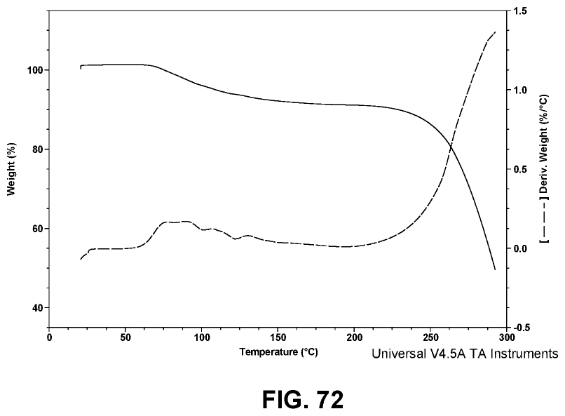

In some embodiments, the Compound II free form EtOH Solvate Form B is characterized by a TGA thermogram substantially similar to that in FIG. 72.

In some embodiments, the Compound II free form EtOH Solvate Form B is characterized by a DSC curve having endothermic peaks at about 67° C. and 105° C.

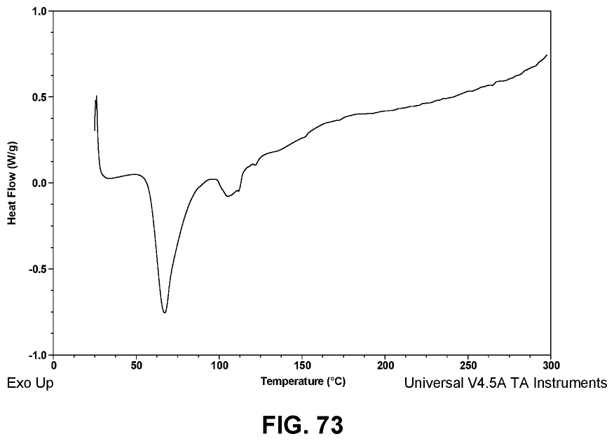

In some embodiments, the Compound II free form EtOH Solvate Form B is characterized by a DSC curve substantially similar to that in FIG. 73.

Some embodiments of the disclosure provide a method of preparing Compound II free form EtOH Solvate Form B comprising:

slowly evaporating Compound II in EtOH at 4° C.; and

isolating the solids.

### Compound II Free Form IPA Solvate

Some embodiments of the disclosure provide an IPA solvate form of Compound II (Compound II free form IPA Solvate). In some embodiments, the Compound II free form IPA Solvate is substantially pure.

In some embodiments, the Compound II free form IPA Solvate is characterized by an X-ray powder diffractogram comprising a signal at 8.4±0.2 two-theta. In some embodiments, the Compound II free form IPA Solvate is characterized by an X-ray powder diffractogram comprising a signal at 11.7±0.2 two-theta. In some embodiments, the Compound II free form IPA Solvate is characterized by an X-ray powder diffractogram comprising a signal at 21.6±0.2 two-theta. In some embodiments, the Compound II free form IPA Solvate is characterized by an X-ray powder diffractogram comprising a signal at 23.3±0.2 two-theta.

In some embodiments, the Compound II free form IPA Solvate is characterized by an X-ray powder diffractogram comprising signals at two or more two-theta values chosen from 8.4±0.2, 11.7±0.2, 21.6±0.2, and 23.3±0.2. In some embodiments, the Compound II free form IPA Solvate is characterized by an X-ray powder diffractogram comprising signals at three or more two-theta values chosen from 8.4±0.2, 11.7±0.2, 21.6±0.2, and 23.3±0.2. In some embodiments, the Compound II free form IPA Solvate is characterized by an X-ray powder diffractogram comprising a signal at 8.4±0.2 two-theta, 11.7±0.2 two-theta, 21.6±0.2 two-theta, and 23.3±0.2 two-theta.

In some embodiments, the Compound II free form IPA Solvate is characterized by an X-ray powder diffractogram comprising (a) signals at two or more two-theta values chosen from 8.4±0.2, 11.7±0.2, 21.6±0.2, and 23.3±0.2; and (b) a signal at one or more (e.g., two, three, or four) two-theta values chosen from 17.0±0.2, 19.9±0.2, 21.9±0.2 and 22.1±0.2. In some embodiments, the Compound II free form IPA Solvate is characterized by an X-ray powder diffractogram comprising (a) signals at three or more two-theta values chosen from 8.4±0.2, 11.7±0.2, 21.6±0.2, and 23.3±0.2; and (b) signals at one or more (e.g., two, three, or four) two-theta values chosen from 17.0±0.2, 19.9±0.2, 21.9±0.2 and 22.1±0.2. In some embodiments, the Compound II free form IPA Solvate is characterized by an X-ray powder diffractogram comprising (a) signals at 8.4±0.2 two-theta, 11.7±0.2 two-theta, 21.6±0.2 two-theta, and 23.3±0.2 two-theta; and (b) a signal at one or more (e.g., two, three, or four) two-theta values chosen from 17.0±0.2, 19.9±0.2, 21.9±0.2 and 22.1±0.2.

In some embodiments, the Compound II free form IPA Solvate is characterized by an X-ray powder diffractogram comprising signals at 8.4±0.2 two-theta, 11.7±0.2 two-theta, 17.0±0.2 two-theta, 19.9±0.2 two-theta, 21.6±0.2 two-theta, 21.9±0.2 two-theta, 22.1±0.2 two-theta, and 23.3±0.2 two-theta.

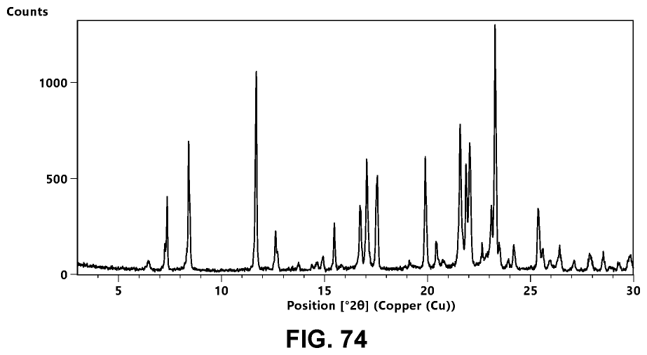

In some embodiments, the Compound II free form IPA Solvate is characterized by an X-ray powder diffractogram substantially similar to that in FIG. 74.

In some embodiments, the Compound II free form IPA Solvate is characterized by a ¹³C NMR spectrum comprising one or more signals chosen from 147.5±0.2 ppm, 74.5±0.2 ppm, and 49.5±0.2 ppm. In some embodiments, the Compound II free form IPA Solvate is characterized by a ¹³C NMR spectrum comprising two or more signals chosen from 147.5±0.2 ppm, 74.5±0.2 ppm, and 49.5±0.2 ppm. In some embodiments, the Compound II free form IPA Solvate is characterized by a ¹³C NMR spectrum comprising signals at 147.5±0.2 ppm, 74.5±0.2 ppm, and 49.5±0.2 ppm.

In some embodiments, the Compound II free form IPA Solvate is characterized by a ¹³C NMR spectrum comprising one or more (e.g., two, three, four, five, six, seven, eight, nine, or more) signals chosen from 147.5±0.2 ppm, 143.0±0.2 ppm, 74.9±0.2 ppm, 74.5±0.2 ppm, 61.7±0.2 ppm 49.5±0.2 ppm, 48.9±0.2 ppm, 22.4±0.2 ppm, 22.0±0.2 ppm, 21.7±0.2 ppm. In some embodiments, the Compound II free form IPA Solvate is characterized by a ¹³C NMR spectrum comprising signals at 147.5±0.2 ppm, 143.0±0.2 ppm, 74.9±0.2 ppm, 74.5±0.2 ppm, 61.7±0.2 ppm, 49.5±0.2 ppm, 48.9±0.2 ppm, 22.4±0.2 ppm, 22.0±0.2 ppm, 21.7±0.2 ppm.

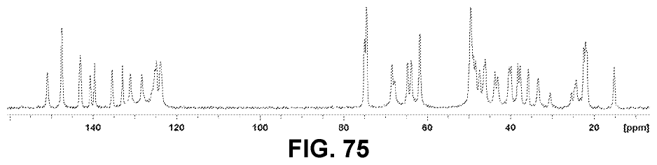

In some embodiments, the Compound II free form IPA Solvate is characterized by a ¹³C NMR spectrum substantially similar to that in FIG. 75.

Some embodiments of the disclosure provide a method of preparing Compound II free form IPA Solvate comprising:

making a slurry of Compound II free form Hemihydrate Form A in 50/50 IPA/heptane (vol/vol);

shaking overnight in a shaker block at 20° C. and 1000 rpm; and

isolating the solids.

### Compound II Free Form MEK Solvate

Some embodiments of the disclosure provide an MEK solvate form of Compound II (Compound II free form MEK Solvate). In some embodiments, the Compound II free form MEK Solvate is substantially pure.

In some embodiments, the Compound II free form MEK Solvate is characterized by a ¹³C NMR spectrum comprising one or more (e.g., two, three, four, five, six, or more) signals chosen from 8.2±0.2 ppm, 23.2±0.2 ppm, 30.0±0.2 ppm, 35.0±0.2 ppm, 35.7±0.2 ppm 39.3±0.2 ppm, and 63.3±0.2 ppm. In some embodiments, the Compound II free form MEK Solvate is characterized by a ¹³C NMR spectrum comprising signals at 8.2±0.2 ppm, 23.2±0.2 ppm, 30.0±0.2 ppm, 35.0±0.2 ppm, 35.7±0.2 ppm 39.3±0.2 ppm, and 63.3±0.2 ppm.

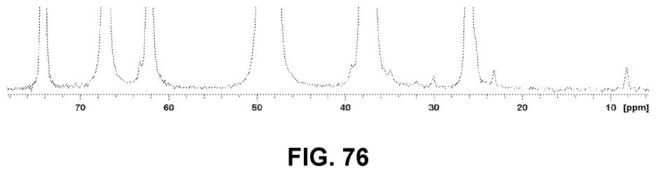

In some embodiments, the Compound II free form MEK Solvate is characterized by a ¹³C NMR spectrum substantially similar to that in FIG. 76.

Some embodiments of the disclosure provide a method of preparing Compound II free form MEK Solvate comprising:

charging Compound II free form Hemihydrate Form A to a jacketed reactor and adding methyl ethyl ketone;

agitating at 300 rpm in a reactor at 45° C.;

adding Compound II free form Hemihydrate Form A as seeds and holding at 45° C. for 30 minutes;

cooling to 20° C. for 1 hour; and

isolating the solids.

### Compound II Free Form Meoh Solvate

Some embodiments of the disclosure provide an MeOH solvate form of Compound II (Compound II free form MeOH Solvate). In some embodiments, the Compound II free form MeOH Solvate is substantially pure.

In some embodiments, the Compound II free form MeOH Solvate is characterized by an X-ray powder diffractogram comprising a signal at 13.4±0.2 two-theta. In some embodiments, the Compound II free form MeOH Solvate is characterized by an X-ray powder diffractogram comprising a signal at 16.6±0.2 two-theta. In some embodiments, the Compound II free form MeOH Solvate is characterized by an X-ray powder diffractogram comprising a signal at 24.3±0.2 two-theta. In some embodiments, the Compound II free form MeOH Solvate is characterized by an X-ray powder diffractogram comprising a signal at 24.4±0.2 two-theta. In some embodiments, the Compound II free form MeOH Solvate is characterized by an X-ray powder diffractogram comprising a signal at 26.3±0.2 two-theta.

In some embodiments, the Compound II free form MeOH Solvate is characterized by an X-ray powder diffractogram comprising a signal at two or more two-theta values chosen from 13.4±0.2, 16.6±0.2, 24.3±0.2, 24.4±0.2, and 26.3±0.2. In some embodiments, the Compound II free form MeOH Solvate is characterized by an X-ray powder diffractogram comprising a signal at three or more two-theta values chosen from 13.4±0.2, 16.6±0.2, 24.3±0.2, 24.4±0.2, and 26.3±0.2. In some embodiments, the Compound II free form MeOH Solvate is characterized by an X-ray powder diffractogram comprising a signal at four or more two-theta values chosen from 13.4±0.2, 16.6±0.2, 24.3±0.2, 24.4±0.2, and 26.3±0.2. In some embodiments, the Compound II free form MeOH Solvate is characterized by an X-ray powder diffractogram comprising a signal at 13.4±0.2 two-theta, 16.6±0.2 two-theta, 24.3±0.2 two-theta, 24.4±0.2 two-theta, and 26.3±0.2 two-theta.

In some embodiments, the Compound II free form MeOH Solvate is characterized by an X-ray powder diffractogram comprising (a) a signal at two or more two-theta values chosen from 13.4±0.2, 16.6±0.2, 24.3±0.2, 24.4±0.2, and 26.3±0.2; and (b) a signal at one or more (e.g., two, three, or four) two-theta values chosen from 12.0±0.2, 21.2±0.2, 24.1±0.2, and 24.2±0.2. In some embodiments, the Compound II free form MeOH Solvate is characterized by an X-ray powder diffractogram comprising (a) a signal at three or more two-theta values chosen from 13.4±0.2, 16.6±0.2, 24.3±0.2, 24.4±0.2, and 26.3±0.2; and (b) a signal at one or more (e.g., two, three, or four) two-theta values chosen from 12.0±0.2, 21.2±0.2, 24.1±0.2, and 24.2±0.2. In some embodiments, the Compound II free form MeOH Solvate is characterized by an X-ray powder diffractogram comprising (a) a signal at 13.4±0.2 two-theta, 16.6±0.2 two-theta, 24.3±0.2 two-theta, 24.4±0.2 two-theta, and 26.3±0.2 two-theta; and (b) a signal at one or more (e.g., two, three, or four) two-theta values chosen from 12.0±0.2, 21.2±0.2, 24.1±0.2, and 24.2±0.2.

In some embodiments, the Compound II free form MeOH Solvate is characterized by an X-ray powder diffractogram comprising a signal 12.0±0.2 two-theta, 13.4±0.2 two-theta, 16.6±0.2 two-theta, 21.2±0.2 two-theta, 24.1±0.2 two-theta, and 24.2±0.2, 24.3±0.2 two-theta, 24.4±0.2 two-theta.

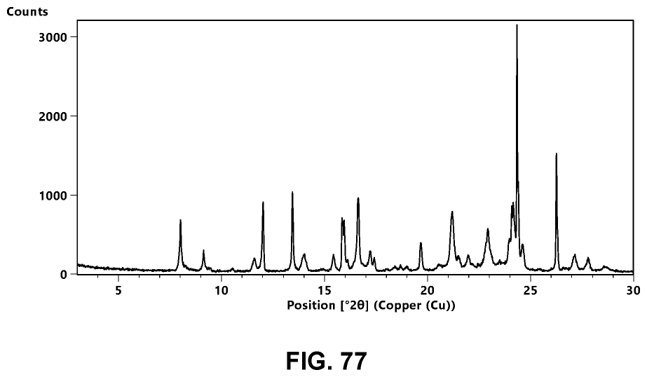

In some embodiments, the Compound II free form MeOH Solvate is characterized by an X-ray powder diffractogram substantially similar to that in FIG. 77.

In some embodiments, the Compound II free form MeOH Solvate is characterized by a TGA thermogram showing 0.87% weight loss from ambient temperature up to 150° C.

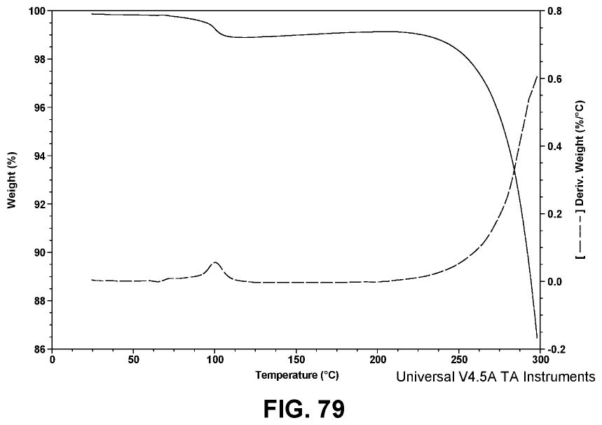

In some embodiments, the Compound II free form MeOH Solvate is characterized by a TGA thermogram substantially similar to that in FIG. 79.

In some embodiments, the Compound II free form MeOH Solvate is characterized by a DSC curve having endothermic peaks at about 79° C., 112° C., and 266° C.

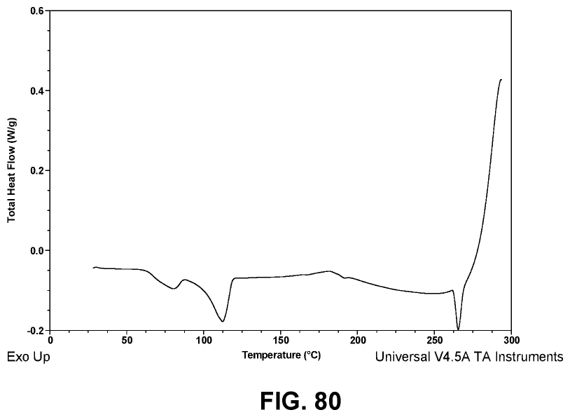

In some embodiments, the Compound II free form MeOH Solvate is characterized by a DSC curve substantially similar to that in FIG. 80.

In some embodiments, the Compound II free form MeOH Solvate is characterized by a ¹³C NMR spectrum comprising one or more (e.g., two, three, four, five, or six) signals chosen from 133.6±0.2 ppm, 74.8±0.2 ppm, 67.7±0.2 ppm, 62.6±0.2 ppm, 49.8±0.2 ppm, and 21.2±0.2 ppm. In some embodiments, the Compound II free form MeOH Solvate is characterized by a ¹³C NMR spectrum comprising signals at 133.6±0.2 ppm, 74.8±0.2 ppm, 67.7±0.2 ppm, 62.6±0.2 ppm, 49.8±0.2 ppm, and 21.2±0.2 ppm.

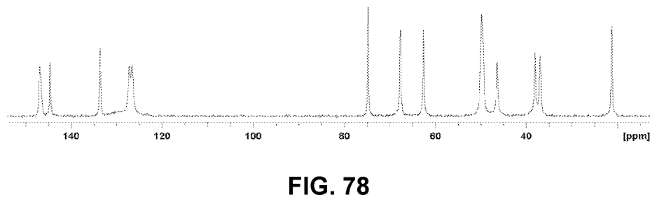

In some embodiments, the Compound II free form MeOH Solvate is characterized by a ¹³C NMR spectrum substantially similar to that in FIG. 78.

In some embodiments, the Compound II free form MeOH Solvate is characterized by a single crystal unit cell characterized by a monoclinic crystal system, a C2 space group, and unit cell dimensions measured at 100 K on a Bruker diffractometer equipped Cu Kα radiation (λ=1.54178 Å) of:

| | | | |
|---|---|---|---|
| a | 22.2 ± 0.1 Å | α | 90° |
| b | 7.8 ± 0.1 Å | β | 114.5 ± 0.1° |
| c | 11.9 ± 0.1 Å | γ | 90°. |

Some embodiments of the disclosure provide a method of preparing Compound II free form MeOH Solvate comprising:

mixing Amorphous free form Compound II with MeOH followed by rotary evaporation; and

isolating the solids.

### Amorphous Free Form Compound II

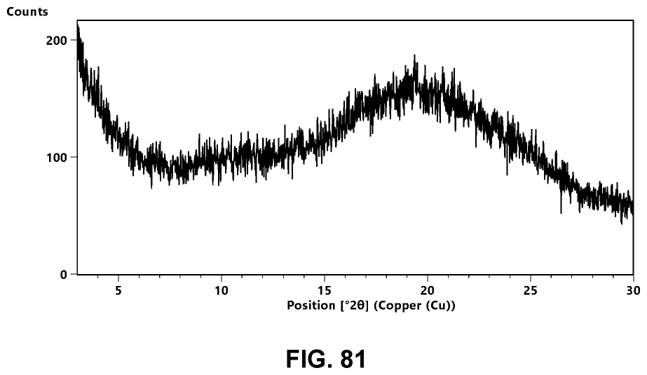

Some embodiments of the disclosure provide an amorphous form of Compound II (Amorphous free form Compound II). In some embodiments, the Amorphous free form Compound II is substantially pure. In some embodiments, the Amorphous free form Compound II is characterized by an X-ray powder diffractogram substantially similar to that in FIG. 81.

In some embodiments, the Amorphous free form Compound II is characterized by a TGA thermogram showing 0.7% weight loss from ambient temperature up to 150° C.

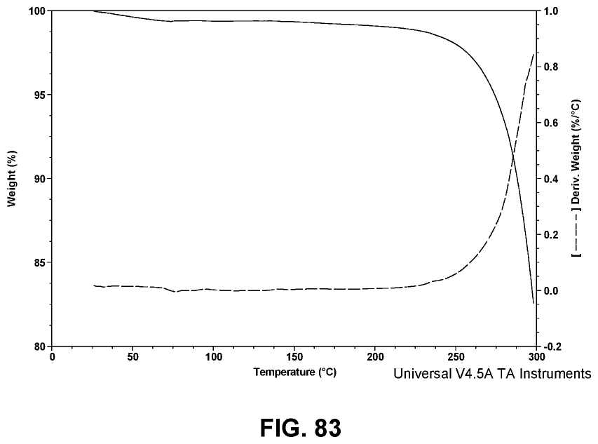

In some embodiments, the Amorphous free form Compound II is characterized by a TGA thermogram substantially similar to that in FIG. 83.

In some embodiments, the Amorphous free form Compound II is characterized by a DSC curve showing glass transition about 78-88° C.

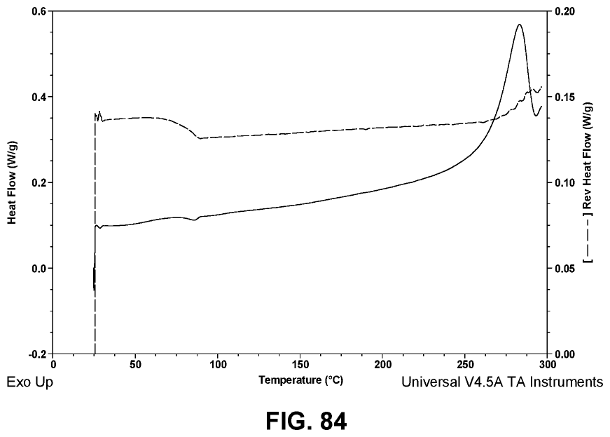

In some embodiments, the Amorphous free form Compound II is characterized by a DSC curve substantially similar to that in FIG. 84.

In some embodiments, the Amorphous free form Compound II is characterized by a ¹³C NMR spectrum comprising one or more (e.g., two, three, or four) signals chosen from 74.3±0.2 ppm, 63.0±0.2 ppm, 48.2±0.2 ppm, and 37.2±0.2 ppm. In some embodiments, the Amorphous free form Compound II is characterized by a ¹³C NMR spectrum comprising signals at 74.3±0.2 ppm, 63.0±0.2 ppm, 48.2±0.2 ppm, and 37.2±0.2 ppm.

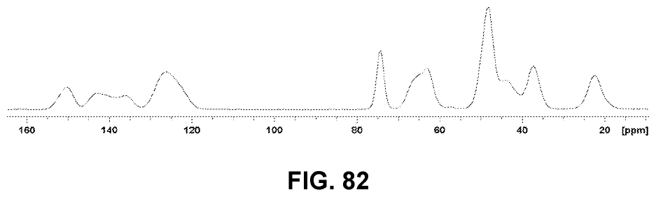

In some embodiments, the Compound II free form MeOH Solvate is characterized by a ¹³C NMR spectrum substantially similar to that in FIG. 82.

### Compound II Phosphate Salt Acetone Solvate Form A

Some embodiments of the disclosure provide a phosphate salt, acetone solvate form of Compound II (Compound II Phosphate Salt Acetone Solvate Form A). In some embodiments, the Compound II Phosphate Salt Acetone Solvate Form A is substantially pure.

In some embodiments, the Compound II Phosphate Salt Acetone Solvate Form A is characterized by an X-ray powder diffractogram comprising a signal at 8.7±0.2 two-theta. In some embodiments, the Compound II Phosphate Salt Acetone Solvate Form A is characterized by an X-ray powder diffractogram comprising a signal at 9.4±0.2 two-theta. In some embodiments, the Compound II Phosphate Salt Acetone Solvate Form A is characterized by an X-ray powder diffractogram comprising a signal at 15.0±0.2 two-theta. In some embodiments, the Compound II Phosphate Salt Acetone Solvate Form A is characterized by an X-ray powder diffractogram comprising a signal at 18.4±0.2 two-theta.

In some embodiments, the Compound II Phosphate Salt Acetone Solvate Form A is characterized by an X-ray powder diffractogram comprising a signal at two or more two-theta values chosen from 8.7±0.2, 9.4±0.2, 15.0±0.2, and 18.4±0.2. In some embodiments, the Compound II Phosphate Salt Acetone Solvate Form A is characterized by an X-ray powder diffractogram comprising a signal at three or more two-theta values chosen from 8.7±0.2, 9.4±0.2, 15.0±0.2, and 18.4±0.2. In some embodiments, the Compound II Phosphate Salt Acetone Solvate Form A is characterized by an X-ray powder diffractogram comprising a signal at 8.7±0.2 two-theta, 9.4±0.2 two-theta, 15.0±0.2 two-theta, and 18.4±0.2 two-theta.

In some embodiments, the Compound II Phosphate Salt Acetone Solvate Form A is characterized by an X-ray powder diffractogram comprising (a) a signal at 8.7±0.2 two-theta, 9.4±0.2 two-theta, 15.0±0.2 two-theta, and 18.4±0.2 two-theta; and (b) a signal at one or more two-theta values chosen from 10.4±0.2, 18.8±0.2, 20.8±0.2, and 22.6±0.2. In some embodiments, the Compound II Phosphate Salt Acetone Solvate Form A is characterized by an X-ray powder diffractogram comprising (a) a signal at 8.7±0.2 two-theta, 9.4±0.2 two-theta, 15.0±0.2 two-theta, and 18.4±0.2 two-theta; and (b) a signal at two or more two-theta values chosen from 10.4±0.2, 18.8±0.2, 20.8±0.2, and 22.6±0.2. In some embodiments, the Compound II Phosphate Salt Acetone Solvate Form A is characterized by an X-ray powder diffractogram comprising (a) a signal at 8.7±0.2 two-theta, 9.4±0.2 two-theta, 15.0±0.2 two-theta, and 18.4±0.2 two-theta; and (b) a signal at three or more (e.g., two, three, or four) two-theta values chosen from 10.4±0.2, 18.8±0.2, 20.8±0.2, and 22.6±0.2.

In some embodiments, the Compound II Phosphate Salt Acetone Solvate Form A is characterized by an X-ray powder diffractogram comprising a signal 8.7±0.2 two-theta, 9.4±0.2 two-theta, 10.4±0.2 two-theta, 15.0±0.2 two-theta, 18.4±0.2 two-theta 18.8±0.2 two-theta, 20.8±0.2 two-theta, and 22.6±0.2 two-theta.

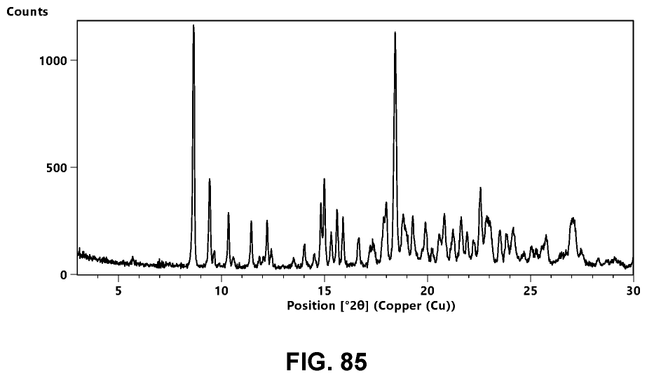

In some embodiments, the Compound II Phosphate Salt Acetone Solvate Form A is characterized by an X-ray powder diffractogram substantially similar to that in FIG. 85.

In some embodiments, the Compound II Phosphate, Acetone Solvate is characterized by a TGA thermogram showing 0.9% weight loss from ambient temperature up to 200° C.

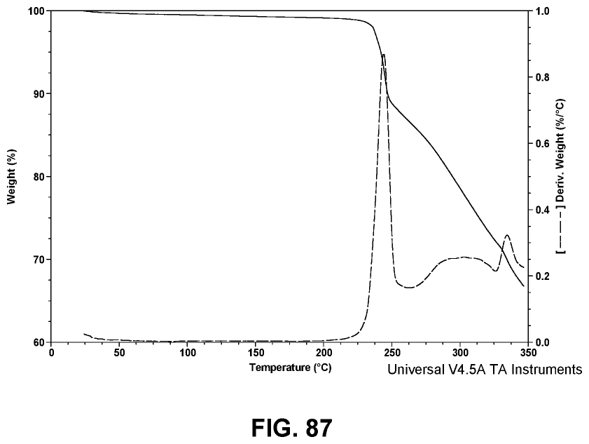

In some embodiments, the Compound II Phosphate, Acetone Solvate is characterized by a TGA thermogram substantially similar to that in FIG. 87.

In some embodiments, the Compound II Phosphate, Acetone Solvate is characterized by a DSC curve having an endothermic peak at about 242° C.

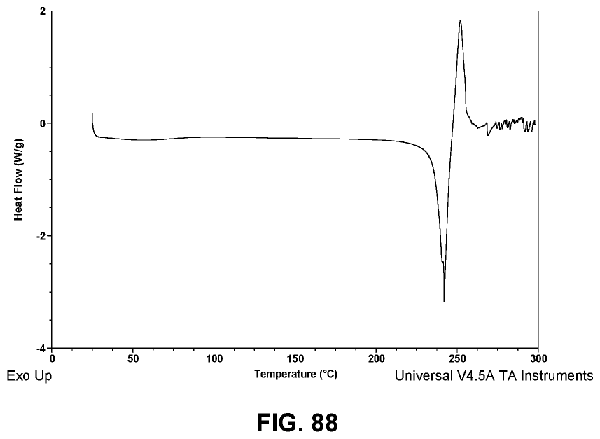

In some embodiments, the Compound II Phosphate, Acetone Solvate is characterized by a DSC curve substantially similar to that in FIG. 88.

In some embodiments, the Compound II Phosphate, Acetone Solvate is characterized by a ¹³C NMR spectrum comprising one or more (e.g., two, three, four, five, six, seven, eight, nine, or ten) signals chosen from 142.3±0.2 ppm, 126.3±0.2 ppm, 73.0±0.2 ppm, 72.3±0.2 ppm, 64.8±0.2 ppm, 62.0±0.2 ppm, 49.4±0.2 ppm, 47.9±0.2 ppm, and 38.2±0.2 ppm. In some embodiments, the Compound II Phosphate, Acetone Solvate is characterized by a ¹³C NMR spectrum comprising signals at 142.3±0.2 ppm, 126.3±0.2 ppm, 73.0±0.2 ppm, 72.3±0.2 ppm, 64.8±0.2 ppm, 62.0±0.2 ppm, 49.4±0.2 ppm, 47.9±0.2 ppm, and 38.2±0.2 ppm.

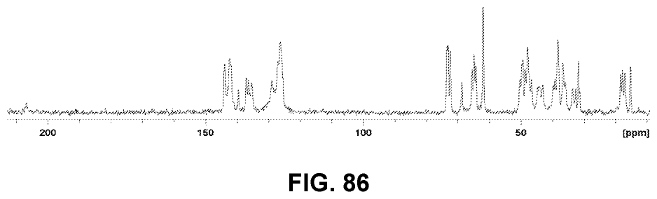

In some embodiments, the Compound II Phosphate, Acetone Solvate is characterized by a ¹³C NMR spectrum substantially similar to that in FIG. 86.

Some embodiments of the disclosure provide a method of preparing Compound II Phosphate, Acetone Solvate comprising:

(a) combining Compound II Phosphate Salt Hemihydrate Form A to a mixture of acetone and water,

stirring at ambient temperature for three days, and

isolating the solids; or

(b) adding Compound II Phosphate Salt Hemihydrate Form A to a mixture of acetone and water at room temperature to form a suspension;

stirring overnight and filtering to obtain a clear saturated solution;

adding equal amounts of Compound II Phosphate Salt Hemihydrate Form A and Compound II Phosphate Salt Form C to the saturated solution;

stirring at ambient temperature for 4 days; and

isolating the solids.

### Compound II Phosphate Salt Form A

Some embodiments of the disclosure provide a phosphate salt form of Compound II (Compound II Phosphate Salt Form A). In some embodiments, the Compound II Phosphate Salt Acetone Solvate Form A is substantially pure.

In some embodiments, the Compound II Phosphate Salt Form A is characterized by an X-ray powder diffractogram comprising a signal at 7.0±0.2 two-theta. In some embodiments, the Compound II Phosphate Salt Form A is characterized by an X-ray powder diffractogram comprising a signal at 9.9±0.2 two-theta. In some embodiments, the Compound II Phosphate Salt Form A is characterized by an X-ray powder diffractogram comprising a signal at 14.1±0.2 two-theta. In some embodiments, the Compound II Phosphate Salt Form A is characterized by an X-ray powder diffractogram comprising a signal at 17.5±0.2 two-theta. In some embodiments, the Compound II Phosphate Salt Form A is characterized by an X-ray powder diffractogram comprising a signal at 19.9±0.2 two-theta.

In some embodiments, the Compound II Phosphate Salt Form A is characterized by an X-ray powder diffractogram comprising a signal at two or more two-theta values chosen from 7.0±0.2, 9.9±0.2, 14.1±0.2, 17.5±0.2 and 19.9±0.2. In some embodiments, the Compound II Phosphate Salt Form A is characterized by an X-ray powder diffractogram comprising a signal at three or more two-theta values chosen from 7.0±0.2, 9.9±0.2, 14.1±0.2, 17.5±0.2 and 19.9±0.2. In some embodiments, the Compound II Phosphate Salt Form A is characterized by an X-ray powder diffractogram comprising a signal at four or more two-theta values chosen from 7.0±0.2±0.2, 9.9±0.2, 14.1±0.2, 17.5±0.2 and 19.9±0.2. In some embodiments, the Compound II Phosphate Salt Form A is characterized by an X-ray powder diffractogram comprising a signal at 7.0±0.2 two-theta, 9.9±0.2 two-theta, 14.1±0.2 two-theta, 17.5±0.2 two-theta, and 19.9±0.2 two-theta.

In some embodiments, the Compound II Phosphate Salt Form A is characterized by an X-ray powder diffractogram comprising (a) a signal at one or more (e.g., two, three, four, or five) two-theta values chosen from 7.0±0.2, 9.9±0.2, 14.1±0.2, 17.5±0.2 and 19.9±0.2; and (b) a signal at one or more two-theta values chosen from 8.9±0.2, 16.9±0.2, 18.5±0.2, and 21.6±0.2. In some embodiments, the Compound II Phosphate Salt Form A is characterized by an X-ray powder diffractogram comprising (a) a signal at one or more (e.g., two, three, four, or five) two-theta values chosen from 7.0±0.2, 9.9±0.2, 14.1±0.2, 17.5±0.2 and 19.9±0.2; and (b) a signal at two or more two-theta values chosen from 8.9±0.2, 16.9±0.2, 18.5±0.2, and 21.6±0.2. In some embodiments, the Compound II Phosphate Salt Form A is characterized by an X-ray powder diffractogram comprising (a) a signal at 7.0±0.2, 9.9±0.2, 14.1±0.2, 17.5±0.2 and 19.9±0.2; and (b) a signal at three or more (e.g., two, three, or four) two-theta values chosen from 8.9±0.2, 16.9±0.2, 18.5±0.2, and 21.6±0.2.

In some embodiments, the Compound II Phosphate Salt Form A is characterized by an X-ray powder diffractogram comprising a signal 7.0±0.2 two-theta, 8.9±0.2, 9.9±0.2 two-theta, 14.1±0.2 two-theta, 16.9±0.2, 17.5±0.2 two-theta, 18.5±0.2, 19.9±0.2 two-theta, and 21.6±0.2 two-theta.

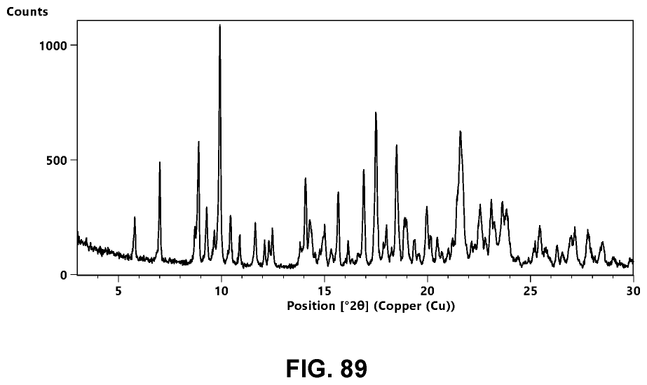

In some embodiments, the Compound II Phosphate Salt Form A is characterized by an X-ray powder diffractogram substantially similar to that in FIG. 89.

In some embodiments, the Compound II Phosphate Salt Form A is characterized by a TGA thermogram showing negligible weight loss from ambient temperature up to 200° C.

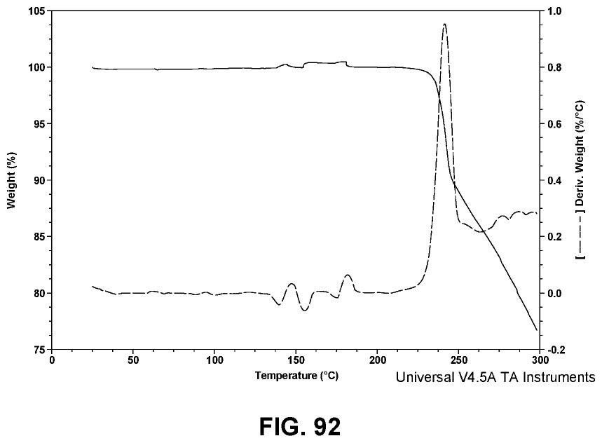

In some embodiments, the Compound II Phosphate Salt Form A is characterized by a TGA thermogram substantially similar to that in FIG. 92.

In some embodiments, the Compound II Phosphate Salt Form A is characterized by a DSC curve having endothermic peaks at about 228° C. and 237° C.

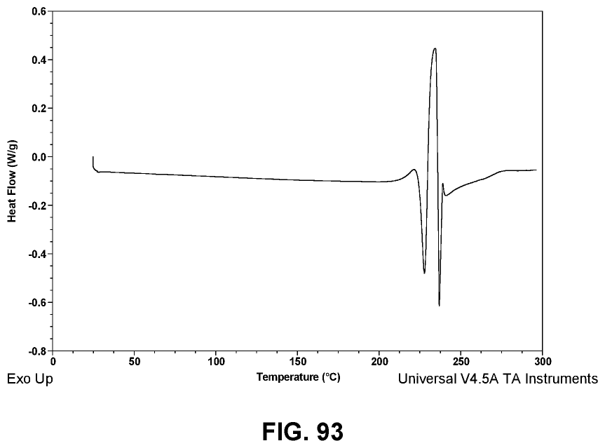

In some embodiments, the Compound II Phosphate Salt Form A is characterized by a DSC curve substantially similar to that in FIG. 93.

In some embodiments, the Compound II Phosphate Salt Form A is characterized by a ¹³C NMR spectrum comprising one or more signals chosen from 72.1±0.2 ppm, 62.0±0.2 ppm, 49.4±0.2 ppm, and 17.5±0.2 ppm. In some embodiments, the Compound II Phosphate Salt Form A is characterized by a ¹³C NMR spectrum comprising two or more signals chosen from 72.1±0.2 ppm, 62.0±0.2 ppm, 49.4±0.2 ppm, and 17.5±0.2 ppm. In some embodiments, the Compound II Phosphate Salt Form A is characterized by a ¹³C NMR spectrum comprising three or more signals chosen from 72.1±0.2 ppm, 62.0±0.2 ppm, 49.4±0.2 ppm, and 17.5±0.2 ppm. In some embodiments, the Compound II Phosphate Salt Form A is characterized by a ¹³C NMR spectrum comprising signals at 72.1±0.2 ppm, 62.0±0.2 ppm, and 49.4±0.2 ppm, and 17.5±0.2 ppm.

In some embodiments, the Compound II Phosphate Salt Form A is characterized by a ¹³C NMR spectrum comprising (a) one or more signals chosen from 72.1±0.2 ppm, 62.0±0.2 ppm, 49.4±0.2 ppm, and 17.5±0.2 ppm; and (b) one or more signals chosen from 72.9±0.2 ppm, 64.4±0.2 ppm, and 64.1±0.2 ppm. In some embodiments, the Compound II Phosphate Salt Form A is characterized by a ¹³C NMR spectrum comprising (a) one or more signals chosen from 72.1±0.2 ppm, 62.0±0.2 ppm, 49.4±0.2 ppm, and 17.5±0.2 ppm; and (b) signals at 72.9±0.2 ppm, 64.4±0.2 ppm, and 64.1±0.2 ppm. In some embodiments, the Compound II Phosphate Salt Form A is characterized by a ¹³C NMR spectrum comprising signals at 72.9±0.2 ppm, 72.1±0.2 ppm, 64.4±0.2 ppm, 64.1±0.2 ppm, 62.0±0.2 ppm, and 49.4±0.2 ppm, and 17.5±0.2 ppm.

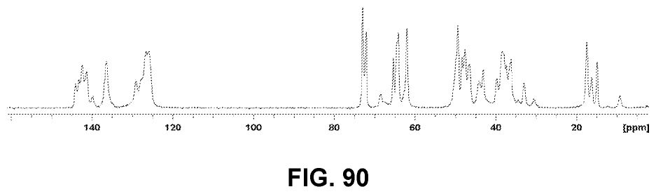

In some embodiments, the Compound II Phosphate Salt Form A is characterized by a ¹³C NMR spectrum substantially similar to that in FIG. 90.

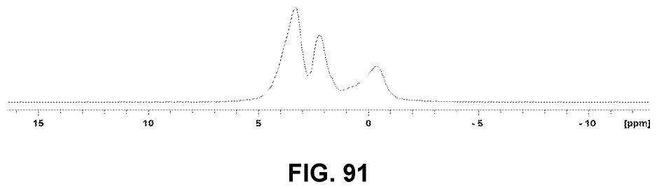

In some embodiments, the Compound II Phosphate Salt Form A is characterized by a ³¹P CPMAS spectrum comprising one or more signals chosen from 3.3±0.2 ppm, 2.2±0.2 ppm, and −0.4±0.2 ppm. In some embodiments, the Compound II Phosphate Salt Form A is characterized by a ³¹P CPMAS spectrum comprising signals at 3.3±0.2 ppm, 2.2±0.2 ppm, and −0.4±0.2 ppm. In some embodiments, the Compound II Phosphate Salt Form A is characterized by a ³¹P CPMAS spectrum substantially similar to that in FIG. 91.

Some embodiments of the disclosure provide a method of preparing Compound II Phosphate Salt Form A comprising:

adding MEK followed by phosphoric acid to Amorphous free form Compound II,

stirring at ambient temperature for 48 hours;

filtering and washing the solids with 4:1 n-heptane/MEK (v/v);

drying in a vacuum oven 18 hours at 60° C.; and

isolating the solids.

### Compound II Phosphate Salt Form C

Some embodiments of the disclosure provide a phosphate salt form of Compound II (Compound II Phosphate Salt Form C). In some embodiments, the Compound II Phosphate, Acetone Solvate Form C is substantially pure.

In some embodiments, the Compound II Phosphate Salt Form C is characterized by an X-ray powder diffractogram comprising a signal at 13.5±0.2 two-theta. In some embodiments, the Compound II Phosphate Salt Form C is characterized by an X-ray powder diffractogram comprising a signal at 13.7±0.2 two-theta. In some embodiments, the Compound II Phosphate Salt Form C is characterized by an X-ray powder diffractogram comprising a signal at 15.0±0.2 two-theta.

In some embodiments, the Compound II Phosphate Salt Form C is characterized by an X-ray powder diffractogram comprising a signal at two or more two-theta values chosen from 13.5±0.2, 13.7±0.2, and 15.0±0.2. In some embodiments, the Compound II Phosphate Salt Form C is characterized by an X-ray powder diffractogram comprising signals at 13.5±0.2±0.2 two-theta, 13.7±0.2 two-theta, and 15.0±0.2 two-theta.

In some embodiments, the Compound II Phosphate Salt Form C is characterized by an X-ray powder diffractogram comprising (a) signals at 13.5±0.2±0.2 two-theta, 13.7±0.2 two-theta, and 15.0±0.2 two-theta; and (b) a signal at one or more two-theta values chosen from 9.1±0.2, 9.4±0.2, 10.4±0.2, 11.0±0.2, and 18.6±0.2. In some embodiments, the Compound II Phosphate Salt Form C is characterized by an X-ray powder diffractogram comprising (a) signals at 13.5±0.2 two-theta, 13.7±0.2 two-theta, and 15.0±0.2 two-theta; and (b) a signal at two or more two-theta values chosen from 9.1±0.2, 9.4±0.2, 10.4±0.2, 11.0±0.2, and 18.6±0.2. In some embodiments, the Compound II Phosphate Salt Form C is characterized by an X-ray powder diffractogram comprising (a) signals at 13.5±0.2 two-theta, 13.7±0.2 two-theta, and 15.0±0.2 two-theta; and (b) a signal at three or more two-theta values chosen from 9.1±0.2, 9.4±0.2, 10.4±0.2, 11.0±0.2, and 18.6±0.2. In some embodiments, the Compound II Phosphate Salt Form C is characterized by an X-ray powder diffractogram comprising signals at 9.1±0.2 two-theta, 9.4±0.2 two-theta, 10.4±0.2 two-theta, 11.0±0.2 two-theta, 13.5±0.2±0.2 two-theta, 13.7±0.2 two-theta, 15.0±0.2 two-theta, and 18.6±0.2 two-theta.

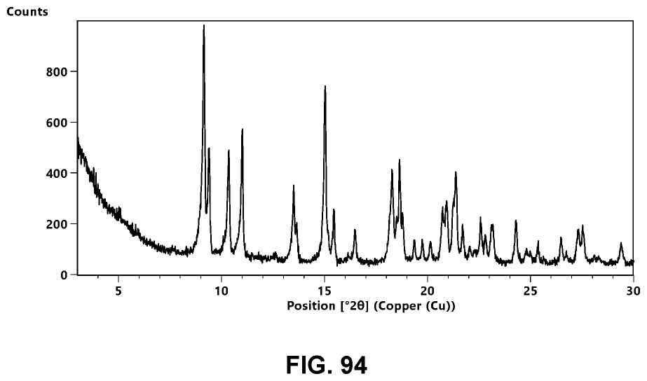

In some embodiments, the Compound II Phosphate Salt Form C is characterized by an X-ray powder diffractogram substantially similar to that in FIG. 94.

In some embodiments, the Compound II Phosphate Salt Form C is characterized by a TGA thermogram showing 1.6% weight loss from ambient temperature up to 150° C.

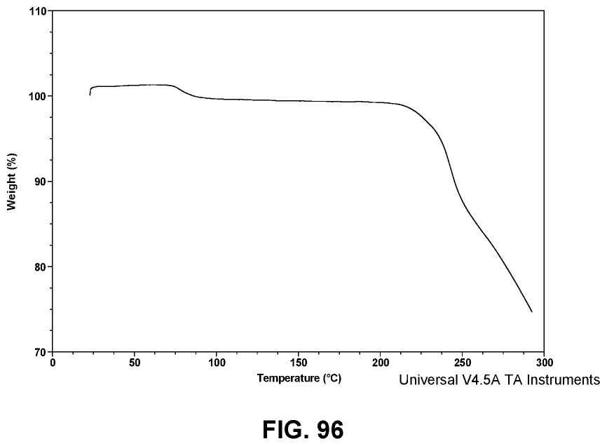

In some embodiments, the Compound II Phosphate Salt Form C is characterized by a TGA thermogram substantially similar to that in FIG. 96.

In some embodiments, the Compound II Phosphate Salt Form C is characterized by a DSC curve having an endothermic peak at about 244° C.

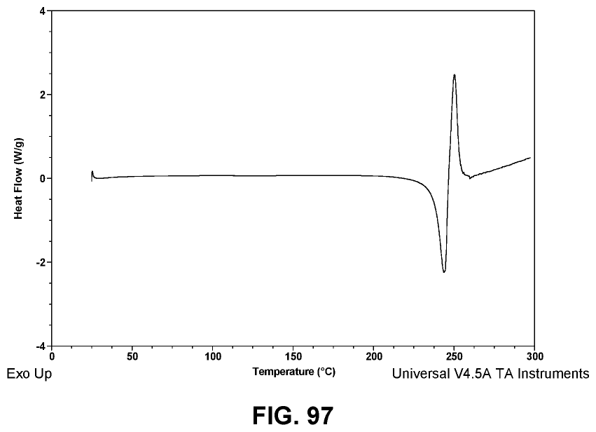

In some embodiments, the Compound II Phosphate Salt Form C is characterized by a DSC curve substantially similar to that in FIG. 97.

In some embodiments, the Compound II Phosphate Salt Form C is characterized by a ¹³C NMR spectrum comprising one or more signals chosen from 139.0±0.2 ppm, 127.8±0.2 ppm, 66.5±0.2 ppm, 62.5±0.2 ppm, and 16.8±0.2 ppm. In some embodiments, the Compound II Phosphate Salt Form C is characterized by a ¹³C NMR spectrum comprising two or more signals chosen from 139.0±0.2 ppm, 127.8±0.2 ppm, 66.5±0.2 ppm, 62.5±0.2 ppm, and 16.8±0.2 ppm. In some embodiments, the Compound II Phosphate Salt Form C is characterized by a ¹³C NMR spectrum comprising three or more signals chosen from 139.0±0.2 ppm, 127.8±0.2 ppm, 66.5±0.2 ppm, 62.5±0.2 ppm, and 16.8±0.2 ppm. In some embodiments, the Compound II Phosphate Salt Form C is characterized by a ¹³C NMR spectrum comprising signals at 139.0±0.2 ppm, 127.8±0.2 ppm, 66.5±0.2 ppm, 62.5±0.2 ppm, and 16.8±0.2 ppm.

In some embodiments, the Compound II Phosphate Salt Form C is characterized by a ¹³C NMR spectrum comprising (a) one or more signals chosen from 139.0±0.2 ppm, 127.8±0.2 ppm, 66.5±0.2 ppm, 62.5±0.2 ppm, and 16.8±0.2 ppm; and (b) one or more (e.g., two, three, four, or five) signals chosen from 143.0±0.2 ppm, 140.3±0.2 ppm, 139.6±0.2 ppm, 72.7±0.2 ppm, 64.1±0.2 ppm, and 47.7±0.2 ppm. In some embodiments, the Compound II Phosphate Salt Form C is characterized by a ¹³C NMR spectrum comprising (a) two or more signals chosen from 139.0±0.2 ppm, 127.8±0.2 ppm, 66.5±0.2 ppm, 62.5±0.2 ppm, and 16.8±0.2 ppm; and (b) one or more (e.g., two, three, four, or five) signals chosen from 143.0±0.2 ppm, 140.3±0.2 ppm, 139.6±0.2 ppm, 72.7±0.2 ppm, 64.1±0.2 ppm, and 47.7±0.2 ppm. In some embodiments, the Compound II Phosphate Salt Form C is characterized by a ¹³C NMR spectrum comprising (a) signals at 139.0±0.2 ppm, 127.8±0.2 ppm, 66.5±0.2 ppm, 62.5±0.2 ppm, and 16.8±0.2 ppm; and (b) one or more (e.g., two, three, four, or five) signals chosen from 143.0±0.2 ppm, 140.3±0.2 ppm, 139.6±0.2 ppm, 72.7±0.2 ppm, 64.1±0.2 ppm, and 47.7±0.2 ppm.

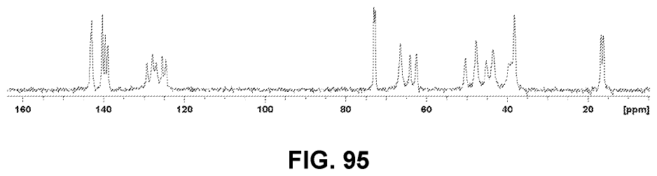

In some embodiments, the Compound II Phosphate Salt Form C is characterized by a ¹³C NMR spectrum substantially similar to that in FIG. 95.

Some embodiments of the disclosure provide a method of preparing Compound II Phosphate Salt Form C comprising:

preparing a slurry of Compound II Phosphate Salt Hemihydrate Form A in 1-butanol at 80° C.; and

centrifuging slurry to isolate the solids.

Another aspect of the disclosure provides pharmaceutical compositions comprising a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I Form B, and Compound I free form Form C. In some embodiments, the pharmaceutical composition comprising a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is administered to a patient in need thereof.

Another aspect of the disclosure provides pharmaceutical compositions comprising a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C. In some embodiments, the pharmaceutical composition comprising a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising a solid form of Compound I chosen from Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I Form B, and Compound I free form Form C or a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C can be administered as a separate composition concurrently with, prior to, or subsequent to, a composition comprising at least one other active therapeutic agent. In some embodiments, a pharmaceutical composition comprising a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C or a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C can be administered as a separate composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the solid forms of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as, e.g., human serum albumin), buffer substances (such as, e.g., phosphates, glycine, sorbic acid, and potassium sorbate), partial glyceride mixtures of saturated vegetable fatty acids, water, salts, and electrolytes (such as, e.g., protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as, e.g., lactose, glucose, and sucrose), starches (such as, e.g., corn starch and potato starch), cellulose and its derivatives (such as, e.g., sodium carboxymethyl cellulose, ethyl cellulose, and cellulose acetate), powdered tragacanth, malt, gelatin, talc, excipients (such as, e.g., cocoa butter and suppository waxes), oils (such as, e.g., peanut oil, cottonseed oil, safflower oil, sesame oil, olive oil, corn oil, and soybean oil), glycols (such as, e.g., propylene glycol and polyethylene glycol), esters (such as, e.g., ethyl oleate and ethyl laurate), agar, buffering agents (such as, e.g., magnesium hydroxide and aluminum hydroxide), alginic acid, pyrogen-free water, isotonic saline, Ringer's solution, ethyl alcohol, phosphate buffer solutions, non-toxic compatible lubricants (such as, e.g., sodium lauryl sulfate and magnesium stearate), coloring agents, releasing agents, coating agents, sweetening agents, flavoring agents, perfuming agents, preservatives, and antioxidants. In some embodiments, the pharmaceutically acceptable carrier is citrate buffer.

In some embodiments, the solid form of Compound I is a crystalline solid consisting of 1% to 99% Compound I Phosphate Salt Hydrate Form A relative to the total weight of the crystalline solid Compound I. In some embodiments, the crystalline solid consists of 2% to 99% Compound I Phosphate Salt Hydrate Form A relative to the total weight of the crystalline solid Compound I. In some embodiments, the crystalline solid consists of 5% to 99% Compound I Phosphate Salt Hydrate Form A relative to the total weight of the crystalline solid Compound I. In some embodiments, the crystalline solid consists of 10% to 99% Compound I Phosphate Salt Hydrate Form A relative to the total weight of the crystalline solid Compound I. In some embodiments, the crystalline solid consists of 15% to 99% Compound I Phosphate Salt Hydrate Form A relative to the total weight of the crystalline solid Compound I. In some embodiments, the crystalline solid consists of 20% to 99% Compound I Phosphate Salt Hydrate Form A relative to the total weight of the crystalline solid Compound I. In some embodiments, the crystalline solid consists of 25% to 99% Compound I Phosphate Salt Hydrate Form A relative to the total weight of the crystalline solid Compound I. In some embodiments, the crystalline solid consists of 30% to 99% Compound I Phosphate Salt Hydrate Form A relative to the total weight of the crystalline solid Compound I. In some embodiments, the crystalline solid consists of 35% to 99% Compound I Phosphate Salt Hydrate Form A relative to the total weight of the crystalline solid Compound I. In some embodiments, the crystalline solid consists of 45% to 99% Compound I Phosphate Salt Hydrate Form A relative to the total weight of the crystalline solid Compound I. In some embodiments, the crystalline solid consists of 50% to 99% Compound I Phosphate Salt Hydrate Form A relative to the total weight of the crystalline solid Compound I. In some embodiments, the crystalline solid consists of 55% to 99% Compound I Phosphate Salt Hydrate Form A relative to the total weight of the crystalline solid Compound I. In some embodiments, the crystalline solid consists of 60% to 99% Compound I Phosphate Salt Hydrate Form A relative to the total weight of the crystalline solid Compound I. In some embodiments, the crystalline solid consists of 65% to 99% Compound I Phosphate Salt Hydrate Form A relative to the total weight of the crystalline solid Compound I. In some embodiments, the crystalline solid consists of 70% to 99% Compound I Phosphate Salt Hydrate Form A relative to the total weight of the crystalline solid Compound I. In some embodiments, the crystalline solid consists of 75% to 99% Compound I Phosphate Salt Hydrate Form A relative to the total weight of the crystalline solid Compound I. In some embodiments, the crystalline solid consists of 80% to 99% Compound I Phosphate Salt Hydrate Form A relative to the total weight of the crystalline solid Compound I. In some embodiments, the crystalline solid consists of 85% to 99% Compound I Phosphate Salt Hydrate Form A relative to the total weight of the crystalline solid Compound I. In some embodiments, the crystalline solid consists of 90% to 99% Compound I Phosphate Salt Hydrate Form A relative to the total weight of the crystalline solid Compound I. In some embodiments, the crystalline solid consists of 95% to 99% Compound I Phosphate Salt Hydrate Form A relative to the total weight of the crystalline solid Compound I.

In some embodiments, the solid form of Compound I is a crystalline solid consisting of 1% to 99% Compound I free form Monohydrate relative to the total weight of the crystalline solid Compound I. In some embodiments, the crystalline solid consists of 2% to 99% Compound I free form Monohydrate relative to the total weight of the crystalline solid Compound I. In some embodiments, the crystalline solid consists of 5% to 99% Compound I free form Monohydrate relative to the total weight of the crystalline solid Compound I. In some embodiments, the crystalline solid consists of 10% to 99% Compound I free form Monohydrate relative to the total weight of the crystalline solid Compound I. In some embodiments, the crystalline solid consists of 15% to 99% Compound I free form Monohydrate relative to the total weight of the crystalline solid Compound I. In some embodiments, the crystalline solid consists of 20% to 99% Compound I free form Monohydrate relative to the total weight of the crystalline solid Compound I. In some embodiments, the crystalline solid consists of 25% to 99% Compound I free form Monohydrate relative to the total weight of the crystalline solid Compound I. In some embodiments, the crystalline solid consists of 30% to 99% Compound I free form Monohydrate relative to the total weight of the crystalline solid Compound I. In some embodiments, the crystalline solid consists of 35% to 99% Compound I free form Monohydrate relative to the total weight of the crystalline solid Compound I. In some embodiments, the crystalline solid consists of 45% to 99% Compound I free form Monohydrate relative to the total weight of the crystalline solid Compound I. In some embodiments, the crystalline solid consists of 50% to 99% Compound I free form Monohydrate relative to the total weight of the crystalline solid Compound I. In some embodiments, the crystalline solid consists of 55% to 99% Compound I free form Monohydrate relative to the total weight of the crystalline solid Compound I. In some embodiments, the crystalline solid consists of 60% to 99% Compound I free form Monohydrate relative to the total weight of the crystalline solid Compound I. In some embodiments, the crystalline solid consists of 65% to 99% Compound I free form Monohydrate relative to the total weight of the crystalline solid Compound I. In some embodiments, the crystalline solid consists of 70% to 99% Compound I free form Monohydrate relative to the total weight of the crystalline solid Compound I. In some embodiments, the crystalline solid consists of 75% to 99% Compound I free form Monohydrate relative to the total weight of the crystalline solid Compound I. In some embodiments, the crystalline solid consists of 80% to 99% Compound I free form Monohydrate relative to the total weight of the crystalline solid Compound I. In some embodiments, the crystalline solid consists of 85% to 99% Compound I free form Monohydrate relative to the total weight of the crystalline solid Compound I. In some embodiments, the crystalline solid consists of 90% to 99% Compound I free form Monohydrate relative to the total weight of the crystalline solid Compound I. In some embodiments, the crystalline solid consists of 95% to 99% Compound I free form Monohydrate relative to the total weight of the crystalline solid Compound I.

In some embodiments, the solid form of Compound I is a crystalline solid consisting of 1% to 99% Compound I Phosphate Salt Methanol Solvate relative to the total weight of the crystalline solid Compound I. In some embodiments, the crystalline solid consists of 2% to 99% Compound I Phosphate Salt Methanol Solvate relative to the total weight of the crystalline solid Compound I. In some embodiments, the crystalline solid consists of 5% to 99% Compound I Phosphate Salt Methanol Solvate relative to the total weight of the crystalline solid Compound I. In some embodiments, the crystalline solid consists of 10% to 99% Compound I Phosphate Salt Methanol Solvate relative to the total weight of the crystalline solid Compound I. In some embodiments, the crystalline solid consists of 15% to 99% Compound I Phosphate Salt Methanol Solvate relative to the total weight of the crystalline solid Compound I. In some embodiments, the crystalline solid consists of 20% to 99% Compound I Phosphate Salt Methanol Solvate relative to the total weight of the crystalline solid Compound I. In some embodiments, the crystalline solid consists of 25% to 99% Compound I Phosphate Salt Methanol Solvate relative to the total weight of the crystalline solid Compound I. In some embodiments, the crystalline solid consists of 30% to 99% Compound I Phosphate Salt Methanol Solvate relative to the total weight of the crystalline solid Compound I. In some embodiments, the crystalline solid consists of 35% to 99% Compound I Phosphate Salt Methanol Solvate relative to the total weight of the crystalline solid Compound I. In some embodiments, the crystalline solid consists of 45% to 99% Compound I Phosphate Salt Methanol Solvate relative to the total weight of the crystalline solid Compound I. In some embodiments, the crystalline solid consists of 50% to 99% Compound I Phosphate Salt Methanol Solvate relative to the total weight of the crystalline solid Compound I. In some embodiments, the crystalline solid consists of 55% to 99% Compound I Phosphate Salt Methanol Solvate relative to the total weight of the crystalline solid Compound I. In some embodiments, the crystalline solid consists of 60% to 99% Compound I Phosphate Salt Methanol Solvate relative to the total weight of the crystalline solid Compound I. In some embodiments, the crystalline solid consists of 65% to 99% Compound I Phosphate Salt Methanol Solvate relative to the total weight of the crystalline solid Compound I. In some embodiments, the crystalline solid consists of 70% to 99% Compound I Phosphate Salt Methanol Solvate relative to the total weight of the crystalline solid Compound I. In some embodiments, the crystalline solid consists of 75% to 99% Compound I Phosphate Salt Methanol Solvate relative to the total weight of the crystalline solid Compound I. In some embodiments, the crystalline solid consists of 80% to 99% Compound I Phosphate Salt Methanol Solvate relative to the total weight of the crystalline solid Compound I. In some embodiments, the crystalline solid consists of 85% to 99% Compound I Phosphate Salt Methanol Solvate relative to the total weight of the crystalline solid Compound I. In some embodiments, the crystalline solid consists of 90% to 99% Compound I Phosphate Salt Methanol Solvate relative to the total weight of the crystalline solid Compound I. In some embodiments, the crystalline solid consists of 95% to 99% Compound I Phosphate Salt Methanol Solvate relative to the total weight of the crystalline solid Compound I.

In some embodiments, the solid form of Compound I is a crystalline solid consisting of 1% to 99% Compound I Phosphate Salt MEK Solvate relative to the total weight of the crystalline solid Compound I. In some embodiments, the crystalline solid consists of 2% to 99% Compound I Phosphate Salt MEK Solvate relative to the total weight of the crystalline solid Compound I. In some embodiments, the crystalline solid consists of 5% to 99% Compound I Phosphate Salt MEK Solvate relative to the total weight of the crystalline solid Compound I. In some embodiments, the crystalline solid consists of 10% to 99% Compound I Phosphate Salt MEK Solvate relative to the total weight of the crystalline solid Compound I. In some embodiments, the crystalline solid consists of 15% to 99% Compound I Phosphate Salt MEK Solvate relative to the total weight of the crystalline solid Compound I. In some embodiments, the crystalline solid consists of 20% to 99% Compound I Phosphate Salt MEK Solvate relative to the total weight of the crystalline solid Compound I. In some embodiments, the crystalline solid consists of 25% to 99% Compound I Phosphate Salt MEK Solvate relative to the total weight of the crystalline solid Compound I. In some embodiments, the crystalline solid consists of 30% to 99% Compound I Phosphate Salt MEK Solvate relative to the total weight of the crystalline solid Compound I. In some embodiments, the crystalline solid consists of 35% to 99% Compound I Phosphate Salt MEK Solvate relative to the total weight of the crystalline solid Compound I. In some embodiments, the crystalline solid consists of 45% to 99% Compound I Phosphate Salt MEK Solvate relative to the total weight of the crystalline solid Compound I. In some embodiments, the crystalline solid consists of 50% to 99% Compound I Phosphate Salt MEK Solvate relative to the total weight of the crystalline solid Compound I. In some embodiments, the crystalline solid consists of 55% to 99% Compound I Phosphate Salt MEK Solvate relative to the total weight of the crystalline solid Compound I. In some embodiments, the crystalline solid consists of 60% to 99% Compound I Phosphate Salt MEK Solvate relative to the total weight of the crystalline solid Compound I. In some embodiments, the crystalline solid consists of 65% to 99% Compound I Phosphate Salt MEK Solvate relative to the total weight of the crystalline solid Compound I. In some embodiments, the crystalline solid consists of 70% to 99% Compound I Phosphate Salt MEK Solvate relative to the total weight of the crystalline solid Compound I. In some embodiments, the crystalline solid consists of 75% to 99% Compound I Phosphate Salt MEK Solvate relative to the total weight of the crystalline solid Compound I. In some embodiments, the crystalline solid consists of 80% to 99% Compound I Phosphate Salt MEK Solvate relative to the total weight of the crystalline solid Compound I. In some embodiments, the crystalline solid consists of 85% to 99% Compound I Phosphate Salt MEK Solvate relative to the total weight of the crystalline solid Compound I. In some embodiments, the crystalline solid consists of 90% to 99% Compound I Phosphate Salt MEK Solvate relative to the total weight of the crystalline solid Compound I. In some embodiments, the crystalline solid consists of 95% to 99% Compound I Phosphate Salt MEK Solvate relative to the total weight of the crystalline solid Compound I.

In some embodiments of the disclosure, a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is used to treat an APOL1 mediated disease (such as, e.g., an APOL1 mediated kidney disease). In some embodiments, the APOL1 mediated disease is chosen from ESKD, FSGS, HIV-associated nephropathy, NDKD, arterionephrosclerosis, lupus nephritis, microalbuminuria, and chronic kidney disease. In some embodiments, the APOL1 mediated disease treated with a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is FSGS. In some embodiments, the APOL1 mediated disease treated with a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is NDKD. In some embodiments, the APOL1 mediated disease treated with a solid form of Compound I chosen from Compound I Phosphate Salt Hydrate Form A, Compound I free form Monohydrate Compound I Maleate Form A (salt or co-crystal), Compound I Maleate Form B (salt or co-crystal), Compound I Fumaric Acid Form A (salt or co-crystal), Compound I Form B, and Compound I free form Form C is ESKD. In some embodiments, the APOL1 mediated disease treated with a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C is cancer. In some embodiments, the APOL1 mediated disease treated with a solid form of Compound I chosen from Compound I Phosphate Salt Hydrate Form A, Compound I free form Monohydrate, Compound I Compound I Maleate Form A (salt or co-crystal), Compound I Maleate Form B (salt or co-crystal), Compound I Fumaric Acid Form A (salt or co-crystal), Compound I Form B, and Compound I free form Form C is pancreatic cancer. In some embodiments, the patient with APOL1 mediated disease (such as, e.g., APOL1 mediated kidney disease) to be treated with a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I free form Form B, and Compound I free form Form C has two APOL1 risk alleles. In some embodiments, the patient with APOL1 mediated disease (such as, e.g., APOL1 mediated kidney disease) is homozygous for APOL1 genetic risk alleles G1: S342G:I384M. In some embodiments, the patient with APOL1 mediated disease (such as, e.g., APOL1 mediated kidney disease) is homozygous for APOL1 genetic risk alleles G2: N388del:Y389del. In some embodiments, the patient with APOL1 mediated disease (such as, e.g., APOL1 mediated kidney disease) is heterozygous for APOL1 genetic risk alleles G1: S342G:I384M and G2: N388del:Y389del.

In some embodiments, the solid form of Compound II is a crystalline solid consisting of 1% to 99% Compound II Phosphate Salt Hemihydrate Form A relative to the total weight of the crystalline solid Compound II. In some embodiments, the crystalline solid consists of 2% to 99% Compound II Phosphate Salt Hemihydrate Form A relative to the total weight of the crystalline solid Compound II. In some embodiments, the crystalline solid consists of 5% to 99% Compound II Phosphate Salt Hemihydrate Form A relative to the total weight of the crystalline solid Compound II. In some embodiments, the crystalline solid consists of 10% to 99% Compound II Phosphate Salt Hemihydrate Form A relative to the total weight of the crystalline solid Compound II. In some embodiments, the crystalline solid consists of 15% to 99% Compound II Phosphate Salt Hemihydrate Form A relative to the total weight of the crystalline solid Compound II. In some embodiments, the crystalline solid consists of 20% to 99% Compound II Phosphate Salt Hemihydrate Form A relative to the total weight of the crystalline solid Compound II. In some embodiments, the crystalline solid consists of 25% to 99% Compound II Phosphate Salt Hemihydrate Form A relative to the total weight of the crystalline solid Compound II. In some embodiments, the crystalline solid consists of 30% to 99% Compound II Phosphate Salt Hemihydrate Form A relative to the total weight of the crystalline solid Compound II. In some embodiments, the crystalline solid consists of 35% to 99% Compound II Phosphate Salt Hemihydrate Form A relative to the total weight of the crystalline solid Compound II. In some embodiments, the crystalline solid consists of 45% to 99% Compound II Phosphate Salt Hemihydrate Form A relative to the total weight of the crystalline solid Compound II. In some embodiments, the crystalline solid consists of 50% to 99% Compound II Phosphate Salt Hemihydrate Form A relative to the total weight of the crystalline solid Compound II. In some embodiments, the crystalline solid consists of 55% to 99% Compound II Phosphate Salt Hemihydrate Form A relative to the total weight of the crystalline solid Compound II. In some embodiments, the crystalline solid consists of 60% to 99% Compound II Phosphate Salt Hemihydrate Form A relative to the total weight of the crystalline solid Compound II. In some embodiments, the crystalline solid consists of 65% to 99% Compound II Phosphate Salt Hemihydrate Form A relative to the total weight of the crystalline solid Compound II. In some embodiments, the crystalline solid consists of 70% to 99% Compound II Phosphate Salt Hemihydrate Form A relative to the total weight of the crystalline solid Compound II. In some embodiments, the crystalline solid consists of 75% to 99% Compound II Phosphate Salt Hemihydrate Form A relative to the total weight of the crystalline solid Compound II. In some embodiments, the crystalline solid consists of 80% to 99% Compound II Phosphate Salt Hemihydrate Form A relative to the total weight of the crystalline solid Compound II. In some embodiments, the crystalline solid consists of 85% to 99% Compound II Phosphate Salt Hemihydrate Form A relative to the total weight of the crystalline solid Compound II. In some embodiments, the crystalline solid consists of 90% to 99% Compound II Phosphate Salt Hemihydrate Form A relative to the total weight of the crystalline solid Compound II. In some embodiments, the crystalline solid consists of 95% to 99% Compound II Phosphate Salt Hemihydrate Form A relative to the total weight of the crystalline solid Compound II.

In some embodiments, the solid form of Compound II is a crystalline solid consisting of 1% to 99% Compound II free form Hemihydrate Form A relative to the total weight of the crystalline solid Compound II. In some embodiments, the crystalline solid consists of 2% to 99% Compound II free form Hemihydrate Form A relative to the total weight of the crystalline solid Compound II. In some embodiments, the crystalline solid consists of 5% to 99% Compound II free form Hemihydrate Form A relative to the total weight of the crystalline solid Compound II. In some embodiments, the crystalline solid consists of 10% to 99% Compound II free form Hemihydrate Form A relative to the total weight of the crystalline solid Compound II. In some embodiments, the crystalline solid consists of 15% to 99% Compound II free form Hemihydrate Form A relative to the total weight of the crystalline solid Compound II. In some embodiments, the crystalline solid consists of 20% to 99% Compound II free form Hemihydrate Form A relative to the total weight of the crystalline solid Compound II. In some embodiments, the crystalline solid consists of 25% to 99% Compound II free form Hemihydrate Form A relative to the total weight of the crystalline solid Compound II. In some embodiments, the crystalline solid consists of 30% to 99% Compound II free form Hemihydrate Form A relative to the total weight of the crystalline solid Compound II. In some embodiments, the crystalline solid consists of 35% to 99% Compound II free form Hemihydrate Form A relative to the total weight of the crystalline solid Compound II. In some embodiments, the crystalline solid consists of 45% to 99% Compound II free form Hemihydrate Form A relative to the total weight of the crystalline solid Compound II. In some embodiments, the crystalline solid consists of 50% to 99% Compound II free form Hemihydrate Form A relative to the total weight of the crystalline solid Compound II. In some embodiments, the crystalline solid consists of 55% to 99% Compound II free form Hemihydrate Form A relative to the total weight of the crystalline solid Compound II. In some embodiments, the crystalline solid consists of 60% to 99% Compound II free form Hemihydrate Form A relative to the total weight of the crystalline solid Compound II. In some embodiments, the crystalline solid consists of 65% to 99% Compound II free form Hemihydrate Form A relative to the total weight of the crystalline solid Compound II. In some embodiments, the crystalline solid consists of 70% to 99% Compound II free form Hemihydrate Form A relative to the total weight of the crystalline solid Compound II. In some embodiments, the crystalline solid consists of 75% to 99% Compound II free form Hemihydrate Form A relative to the total weight of the crystalline solid Compound II. In some embodiments, the crystalline solid consists of 80% to 99% Compound II free form Hemihydrate Form A relative to the total weight of the crystalline solid Compound II. In some embodiments, the crystalline solid consists of 85% to 99% Compound II free form Hemihydrate Form A relative to the total weight of the crystalline solid Compound II. In some embodiments, the crystalline solid consists of 90% to 99% Compound II free form Hemihydrate Form A relative to the total weight of the crystalline solid Compound II. In some embodiments, the crystalline solid consists of 95% to 99% Compound II free form Hemihydrate Form A relative to the total weight of the crystalline solid Compound II.

In some embodiments, the solid form of Compound II is a crystalline solid consisting of 1% to 99% Compound II free form Form C relative to the total weight of the crystalline solid Compound II. In some embodiments, the crystalline solid consists of 2% to 99% Compound II free form Form C relative to the total weight of the crystalline solid Compound II. In some embodiments, the crystalline solid consists of 5% to 99% Compound II free form Form C relative to the total weight of the crystalline solid Compound II. In some embodiments, the crystalline solid consists of 10% to 99% Compound II free form Form C relative to the total weight of the crystalline solid Compound II. In some embodiments, the crystalline solid consists of 15% to 99% Compound II free form Form C relative to the total weight of the crystalline solid Compound II. In some embodiments, the crystalline solid consists of 20% to 99% Compound II free form Form C relative to the total weight of the crystalline solid Compound II. In some embodiments, the crystalline solid consists of 25% to 99% Compound II free form Form C relative to the total weight of the crystalline solid Compound II. In some embodiments, the crystalline solid consists of 30% to 99% Compound II free form Form C relative to the total weight of the crystalline solid Compound II. In some embodiments, the crystalline solid consists of 35% to 99% Compound II free form Form C relative to the total weight of the crystalline solid Compound II. In some embodiments, the crystalline solid consists of 45% to 99% Compound II free form Form C relative to the total weight of the crystalline solid Compound II. In some embodiments, the crystalline solid consists of 50% to 99% Compound II free form Form C relative to the total weight of the crystalline solid Compound II. In some embodiments, the crystalline solid consists of 55% to 99% Compound II free form Form C relative to the total weight of the crystalline solid Compound II. In some embodiments, the crystalline solid consists of 60% to 99% Compound II free form Form C relative to the total weight of the crystalline solid Compound II. In some embodiments, the crystalline solid consists of 65% to 99% Compound II free form Form C relative to the total weight of the crystalline solid Compound II. In some embodiments, the crystalline solid consists of 70% to 99% Compound II free form Form C relative to the total weight of the crystalline solid Compound II. In some embodiments, the crystalline solid consists of 75% to 99% Compound II free form Form C relative to the total weight of the crystalline solid Compound II. In some embodiments, the crystalline solid consists of 80% to 99% Compound II free form Form C relative to the total weight of the crystalline solid Compound II. In some embodiments, the crystalline solid consists of 85% to 99% Compound II free form Form C relative to the total weight of the crystalline solid Compound II. In some embodiments, the crystalline solid consists of 90% to 99% Compound II free form Form C relative to the total weight of the crystalline solid Compound II. In some embodiments, the crystalline solid consists of 95% to 99% Compound II free form Form C relative to the total weight of the crystalline solid Compound II.

In some embodiments of the disclosure, a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C is used to treat an APOL1 mediated disease (such as, e.g., an APOL1 mediated kidney disease). In some embodiments, the APOL1 mediated disease is chosen from ESKD, FSGS, HIV-associated nephropathy, NDKD, arterionephrosclerosis, lupus nephritis, microalbuminuria, and chronic kidney disease. In some embodiments, the APOL1 mediated disease treated with a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form Cis FSGS. In some embodiments, the APOL1 mediated disease treated with a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C is NDKD. In some embodiments, the APOL1 mediated disease treated with a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Compound II free form IPA Solvate, Compound II free form MEK Solvate, Compound II free form MeOH Solvate, Amorphous free form Compound II, Compound II Phosphate Salt Acetone Solvate Form A, Compound II Phosphate Salt Form A, and Compound II Phosphate Salt Form C is ESKD. In some embodiments, the APOL1 mediated disease treated with a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C is cancer. In some embodiments, the APOL1 mediated disease treated with a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C is pancreatic cancer. In some embodiments, the patient with APOL1 mediated disease (such as, e.g., APOL1 mediated kidney disease) to be treated with a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C has two APOL1 risk alleles. In some embodiments, the patient with APOL1 mediated disease (such as, e.g., APOL1 mediated kidney disease) is homozygous for APOL1 genetic risk alleles G1: S342G:I384M. In some embodiments, the patient with APOL1 mediated disease (such as, e.g., APOL1 mediated kidney disease) is homozygous for APOL1 genetic risk alleles G2: N388del:Y389del. In some embodiments, the patient with APOL1 mediated disease (such as, e.g., APOL1 mediated kidney disease) is heterozygous for APOL1 genetic risk alleles G1: S342G:1384M and G2: N388del:Y389del.

In some embodiments, the methods of the disclosure comprise administering a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I Form B, and Compound I free form Form C to a patient in need thereof. In some embodiments, said patient in need thereof possesses APOL1 genetic variants, i.e., G1: S342G:I384M and G2: N388del:Y389del.

In some embodiments, the methods of the disclosure comprise administering a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C to a patient in need thereof. In some embodiments, said patient in need thereof possesses APOL1 genetic variants, i.e., G1: S342G:1384M and G2: N388del:Y389del.

Another aspect of the disclosure provides methods of inhibiting APOL1 activity comprising contacting said APOL1 with a solid form of Compound I chosen from Compound I Phosphate Salt Hydrate Form A, Compound I free form Monohydrate, Compound I Maleate Form A (salt or co-crystal), Compound I Maleate Form B (salt or co-crystal), Compound I Fumaric Acid Form A (salt or co-crystal), Compound I Form B, and Compound I free form Form C.

Another aspect of the disclosure provides methods of inhibiting APOL1 activity comprising contacting said APOL1 with a solid form of Compound II chosen from Compound II Phosphate Salt Hemihydrate Form A, Compound II free form Hemihydrate Form A, Compound II free form Form C, Compound II free form Form A, Compound II free form Form B, Compound II free form Quarter Hydrate, Compound II free form Hydrate Mixture, Compound II free form Monohydrate, Compound II free form Dihydrate, Compound II free form EtOH Solvate Form B, Amorphous free form Compound II, Compound II Phosphate Salt Form A, and Compound II Phosphate Salt Form C.

### Syntheses of Compound I and Compound II

The disclosure features methods for preparing Compound I, Compound II, solid forms of Compound I, and solid forms of Compound II.

In some embodiments, Compound I is prepared according to Scheme 1.

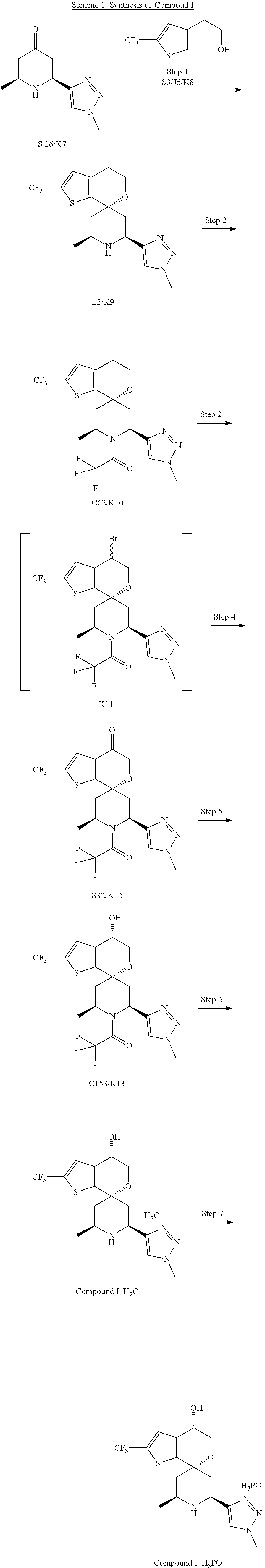

In some embodiments, Compound I is isolated in the form of Compound I.H₂O.

In some embodiments, Compound I is isolated in the form of Compound I I.H₃PO₄.

In some embodiments, Compound I.H₃PO₄ is prepared by converting Compound I.H₂O into Compound I.H₃PO₄.

In some embodiments, converting Compound I.H₂O into Compound I.H₃PO₄ is performed in the presence of methyl ethyl ketone (MEK), water (H₂O), and phosphoric acid (H₃PO₄).

In some embodiments, Compound I.H₃PO₄ is triturated from a 1:1 mixture of MEK/MeOH.

In some embodiments, Compound I.H₂O is prepared by converting Compound C153/K13:

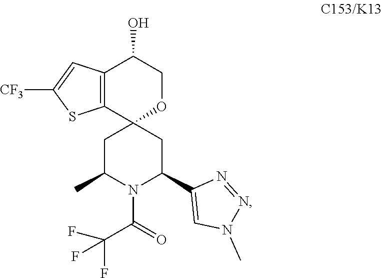 [SMILES: C[C@H]1C[C@@]2(C[C@@H](c3cn(C)nn3)N1C(=O)C(F)(F)F)OC[C@@H](O)c1cc(C(F)(F)F)sc12]

into Compound I.H₂O.

In some embodiments, converting Compound C153/K13 into Compound I.H₂O is performed in the presence of a hydroxide base and a protic solvent.

In some embodiments, converting Compound C153/K13 into Compound I.H₂O is performed in the presence of a hydroxide base selected from lithium hydroxide, sodium hydroxide, and potassium hydroxide.

In some embodiments, converting Compound C153/K13 into Compound I.H₂O is performed in the presence of a protic solvent selected from methanol, ethanol, and 2-propanol.

In some embodiments, converting Compound C153/K13 into Compound I.H₂O is performed in the presence of a hydroxide base and methanol.

In some embodiments, converting Compound C153/K13 into Compound I.H₂O is performed in the presence of sodium hydroxide and a protic solvent.

In some embodiments, converting Compound C153/K13 into Compound I.H₂O is performed in the presence of sodium hydroxide (NaOH) and methanol (MeOH).

In some embodiments, Compound C153/K13 is prepared by converting Compound S32/K12:

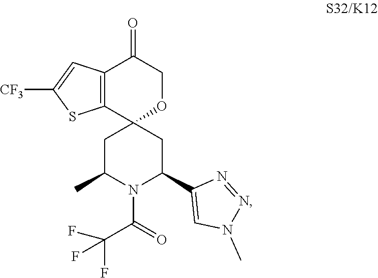 [SMILES: C[C@H]1C[C@@]2(C[C@@H](c3cn(C)nn3)N1C(=O)C(F)(F)F)OCC(=O)c1cc(C(F)(F)F)sc12]

into Compound C153/K13.

In some embodiments, converting Compound C154/K15 into Compound S33/K17 is performed in the presence of cobaltous diacetate tetrahydrate (Co(OAc)₂.4H₂O), N-hydroxyphthalimide, and oxygen (02).

In some embodiments, converting Compound S32/K12 into Compound C153/K13 is performed in the presence of pentamethylcyclopentadienyl rhodium chloride dimer (RhCl₂Cp*)₂, (R,R)—N-(p-toluenesulfonyl)-1,2-diphenylethylenediamine ((R,R)-TsDPEN), formic acid (HCO₂H), and triethylamine (Et₃N).

In some embodiments, converting Compound S32/K12 into Compound C153/K13 is performed in the presence of 0.2 mol % of pentamethylcyclopentadienyl rhodium chloride dimer (RhCl₂Cp*)₂.

In some embodiments, converting Compound S32/K12 into Compound C153/K13 is performed in the presence of 0.05 mol % of pentamethylcyclopentadienyl rhodium chloride dimer (RhCl₂Cp*)₂.

In some embodiments, purifying Compound C153/K13 comprises a rhodium remediation using a resin.

In some embodiments, purifying Compound C153/K13 comprises a rhodium remediation using a DMT resin.

In some embodiments, purifying Compound C153/K13 comprises a rhodium remediation using SiliaMetS® DMT resin.

In some embodiments, purifying Compound C153/K13 comprises a rhodium remediation using Florisil®.

In some embodiments, Compound S32/K12 is prepared by converting Compound C62/K10:

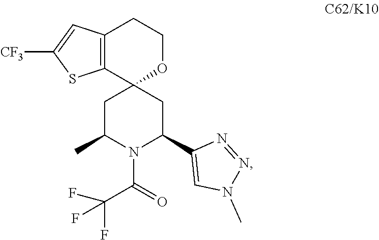 [SMILES: C[C@H]1C[C@@]2(C[C@@H](c3cn(C)nn3)N1C(=O)C(F)(F)F)OCCc1cc(C(F)(F)F)sc12]

into Compound S32/K12.

In some embodiments, converting Compound C62/K10 into Compound S32/K12 comprises:

(i) converting Compound C62/K10 into Compound Kit:

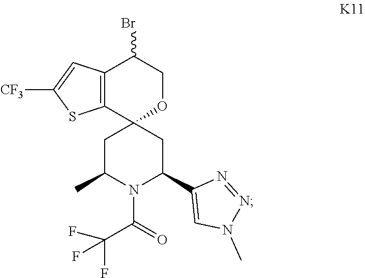

and

(ii) converting Compound K11 into Compound S32/K12.

In some embodiments, converting Compound C62/K10 into Compound K11 is performed in the presence of 1,3-dibromo-5,5-dimethylhydantoin and a radical initiator.

In some embodiments, converting Compound C62/K10 into Compound K11 is performed in the presence of 1,3-dibromo-5,5-dimethylhydantoin and 2,2′-azo-bis-isobutyronitrile (AIBN).

Bromination may also be effected using catalytic ZrCl₄ or ZrBr₄, instead of AIBN, in dichloromethane and other solvents, which allows for a potential decrease in temperature down to 0° C. and removal of AIBN, which is a thermal hazard liability as it has a low thermal onset temperature.

In some embodiments, converting Compound C62/K10 into Compound K11 is performed at 75° C.

In some embodiments, converting Compound C62/K10 into Compound K11 is performed at 50° C.

In some embodiments, converting Compound Kit into Compound S32/K12 is performed in the presence of an amine base.

In some embodiments, converting Compound Kit into Compound S32/K12 is performed in the presence of triethylamine (Et₃N).

In some embodiments, Compound C62/K10 is prepared by converting Compound L2/K9:

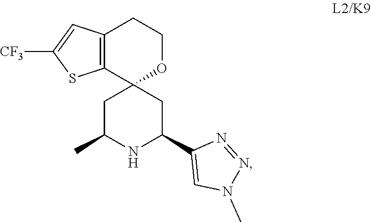 [SMILES: C[C@H]1C[C@@]2(C[C@@H](c3cn(C)nn3)N1)OCCc1cc(C(F)(F)F)sc12]

into Compound C62/K10.

In some embodiments, converting Compound L2/K9 into Compound C62/K10 is performed in the presence of trifluoroacetic anhydride (TFAA) and an amine base.

In some embodiments, converting Compound L2/K9 into Compound C62/K10 is performed in the presence of trifluoroacetic anhydride (TFAA) and N,N-diisopropylethylamine (DIPEA).

In some embodiments, converting Compound L2/K9 into Compound C62/K10 is performed in the presence of trifluoroacetic anhydride (TFAA) and triethylamine (Et₃N).

In some embodiments, Compound L2/K9 is prepared by reacting Compound S26/K7:

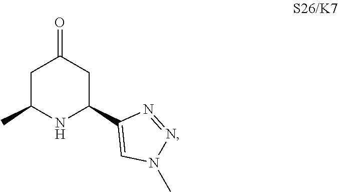

with Compound S3/J6/K8:

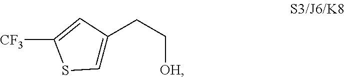 [SMILES: OCCc1csc(C(F)(F)F)c1]

to produce Compound L2/K9.

In some embodiments, reacting Compound S26/K7 with Compound S3/J6/K8 is performed in the presence of an acid.

In some embodiments, reacting Compound S26/K7 with Compound S3/J6/K8 is performed in the presence of a sulfonic acid.

In some embodiments, reacting Compound S26/K7 with Compound S3/J6/K8 is performed in the presence of methanesulfonic acid (MsOH).

In some embodiments, reacting Compound S26/K7 with Compound S3/J6/K8 is performed at 39° C.

In some embodiments, reacting Compound S26/K7 with Compound S3/J6/K8 is performed at 45° C.

In some embodiments, Compound L2/K9 is crystallized using MTBE/n-heptane.

In some embodiments, Compound L2/K9 is crystallized using 9:10 MTBE/n-heptane.

In some embodiments, Compound I is prepared using a compound of the disclosure.

In some embodiments, Compound I is prepared using a compound selected from:

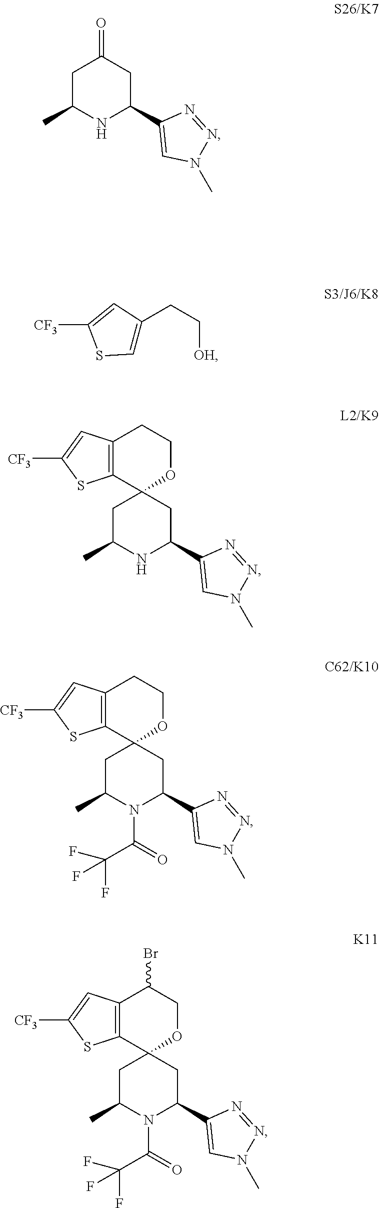

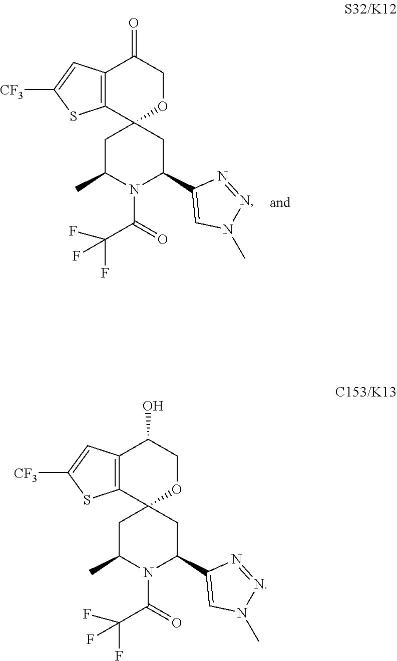 [SMILES: C[C@H]1C[C@@]2(C[C@@H](c3cn(C)nn3)N1C(=O)C(F)(F)F)OCC(=O)c1cc(C(F)(F)F)sc12 ; C[C@H]1C[C@@]2(C[C@@H](c3cn(C)nn3)N1C(=O)C(F)(F)F)OC[C@@H](O)c1cc(C(F)(F)F)sc12]

In some embodiments, a compound of the disclosure is selected from:

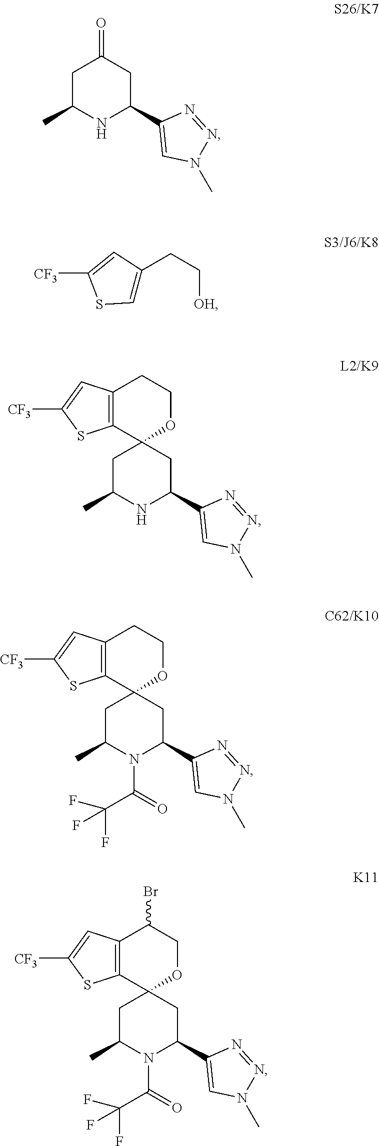

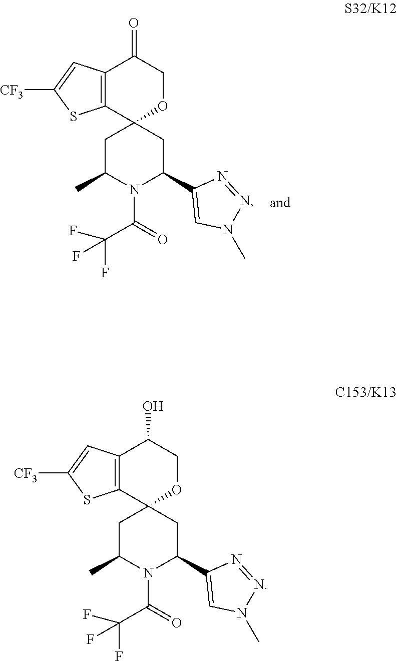 [SMILES: C[C@H]1C[C@@]2(C[C@@H](c3cn(C)nn3)N1C(=O)C(F)(F)F)OCC(=O)c1cc(C(F)(F)F)sc12 ; C[C@H]1C[C@@]2(C[C@@H](c3cn(C)nn3)N1C(=O)C(F)(F)F)OC[C@@H](O)c1cc(C(F)(F)F)sc12]

There are several non-limiting advantages to forming Compound I according to Scheme 1 and the embodiments described above. These advantages are even more apparent when manufacturing Compound I on an industrial scale. The parameters for Step 3 and Step 4 (Scheme 1) have also been improved, resulting in a significant reduction in the reaction temperature and improved process safety profile. The parameters for Step 5 (Scheme 1) have also been optimized, enabling a significant reduction in the amount of rhodium catalyst used and providing a method for rhodium remediation via Florosil®. Finally, Step 6 (Scheme 1) has been improved by adding an optional re-trituration procedure that is used for reducing residual solvents in the product.

In some embodiments, Compound II is prepared according to Scheme 2.

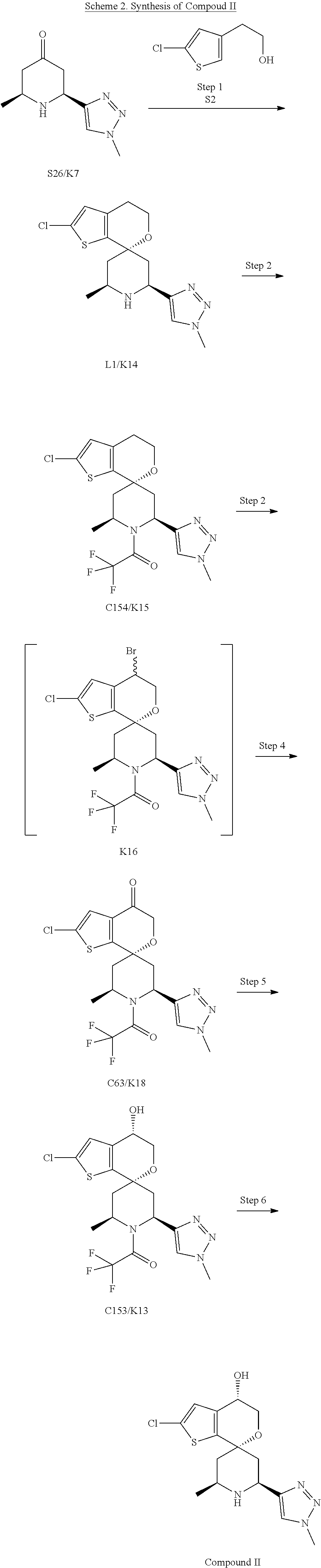

In some embodiments, Compound II is isolated in the form of Compound II free form Form C.

In some embodiments, Compound II is prepared by converting Compound C63/K18:

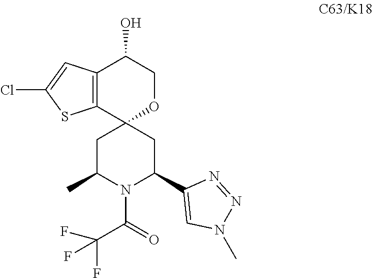 [SMILES: C[C@H]1C[C@@]2(C[C@@H](c3cn(C)nn3)N1C(=O)C(F)(F)F)OC[C@@H](O)c1cc(Cl)sc12]

into Compound II.

In some embodiments, converting Compound C63/K18 into Compound II is performed in the presence of a hydroxide base and a protic solvent.

In some embodiments, converting Compound C63/K18 into Compound II is performed in the presence of a hydroxide base selected from lithium hydroxide, sodium hydroxide, and potassium hydroxide.

In some embodiments, converting Compound C63/K18 into Compound II is performed in the presence of a protic solvent selected from methanol, ethanol, and 2-propanol.

In some embodiments, converting Compound C63/K18 into Compound II is performed in the presence of a hydroxide base and methanol.

In some embodiments, converting Compound C63/K18 into Compound II is performed in the presence of a hydroxide base and 2-propanol.

In some embodiments, converting Compound C63/K18 into Compound II is performed in the presence of sodium hydroxide and a protic solvent.

In some embodiments, converting Compound C63/K18 into Compound II is performed in the presence of sodium hydroxide (NaOH) and methanol (MeOH).

In some embodiments, crystallizing Compound II in the presence of MEK/water produces Compound II free form Hemihydrate Form A.

In some embodiments, converting Compound C63/K18 into Compound II is performed in the presence of sodium hydroxide (NaOH) and 2-propanol.

In some embodiments, crystallizing Compound II in the presence of MEK produces Compound II free form Form C.

In some embodiments, Compound C63/K18 is prepared by converting Compound S33/K17:

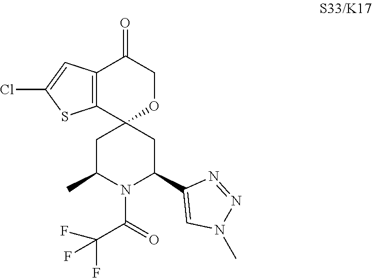 [SMILES: C[C@H]1C[C@@]2(C[C@@H](c3cn(C)nn3)N1C(=O)C(F)(F)F)OCC(=O)c1cc(Cl)sc12]

into Compound C63/K18.

In some embodiments, converting Compound S33/K17 into Compound C63/K18 is performed in the presence of pentamethylcyclopentadienyl rhodium chloride dimer (RhCl₂Cp*)₂, (R,R)—N-(p-toluenesulfonyl)-1,2-diphenylethylenediamine ((R,R)-TsDPEN), formic acid (HCO₂H), and triethylamine (Et₃N).

In some embodiments, converting Compound S33/K17 into Compound C63/K18 is performed in the presence of 0.5 mol % of pentamethylcyclopentadienyl rhodium chloride dimer (RhCl₂Cp*)₂.

In some embodiments, converting Compound S33/K17 into Compound C63/K18 is performed in the presence of 0.05 mol % of pentamethylcyclopentadienyl rhodium chloride dimer (RhCl₂Cp*)₂.

In some embodiments, Compound S33/K17 is prepared by converting Compound C154/K15:

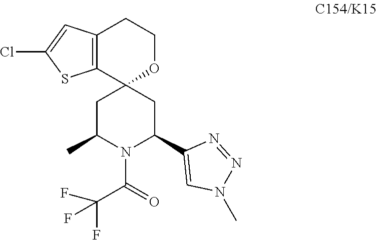 [SMILES: C[C@H]1C[C@@]2(C[C@@H](c3cn(C)nn3)N1C(=O)C(F)(F)F)OCCc1cc(Cl)sc12]

into Compound S33/K17.

In some embodiments, converting Compound C154/K15 into Compound S33/K17 is performed in the presence of cobaltous diacetate tetrahydrate (Co(OAc)₂.4H₂O), N-hydroxyphthalimide, and oxygen (02).

In some embodiments, converting Compound C154/K15 into Compound S33/K17 comprises:

(i) converting Compound C154/K15 into Compound K16:

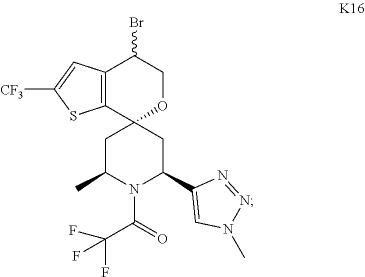

and

(ii) converting Compound K16 into Compound S33/K17.

In some embodiments, converting Compound C154/K15 into Compound K16 is performed in the presence of 1,3-dibromo-5,5-dimethylhydantoin and a radical initiator.

In some embodiments, converting Compound C154/K15 into Compound K16 is performed in the presence of 1,3-dibromo-5,5-dimethylhydantoin and 2,2′-azo-bis-isobutyronitrile (AIBN).

In some embodiments, converting Compound C154/K15 into Compound K16 is performed in chlorobenzene at 75° C.

In some embodiments, converting Compound C154/K15 into Compound K16 is performed in chlorobenzene/1,4-dioxane mixture at 50° C.

In some embodiments, converting Compound K16 into Compound S33/K17 is performed in the presence of triethylamine (Et₃N) and DMSO at 75° C.

In some embodiments, converting Compound K16 into Compound S33/K17 is performed in the presence of triethylamine (Et₃N) and DMSO at 65° C.

In some embodiments, Compound C154/K15 is prepared by converting Compound L1/K14:

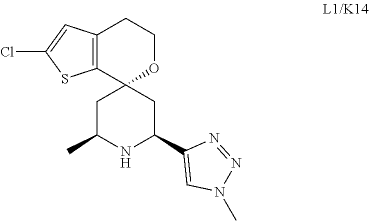 [SMILES: C[C@H]1C[C@@]2(C[C@@H](c3cn(C)nn3)N1)OCCc1cc(Cl)sc12]

into Compound C154/K15.

In some embodiments, converting Compound L1/K14 into Compound C154/K15 is performed in the presence of trifluoroacetic anhydride (TFAA) and N,N-diisopropylethylamine (DIPEA).

In some embodiments, converting Compound L1/K14 into Compound C154/K15 is performed in the presence of trifluoroacetic anhydride (TFAA) and triethylamine (Et₃N).

In some embodiments, Compound L1/K14 is prepared by reacting Compound S26/K7:

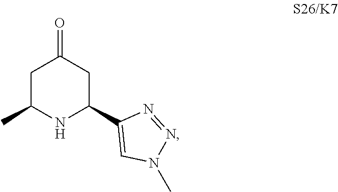

with Compound S2:

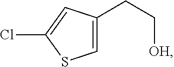

to produce Compound L1/K14.

In some embodiments, reacting Compound S26/K7 with Compound S2 is performed in the presence of an acid.

In some embodiments, reacting Compound S26/K7 with Compound S2 is performed in the presence of a sulfonic acid.

In some embodiments, reacting Compound S26/K7 with Compound S2 is performed in the presence of methanesulfonic acid (MsOH).

In some embodiments, Compound L1/K14 is purified by silica gel chromatography.

In some embodiments, Compound L1/K14 is purified by crystallization from MTBE.

In some embodiments, Compound L1/K14 is purified by crystallization from MTBE/n-heptane.

In some embodiments, Compound II is prepared using a compound of the disclosure.

In some embodiments, Compound II is prepared using a compound selected from:

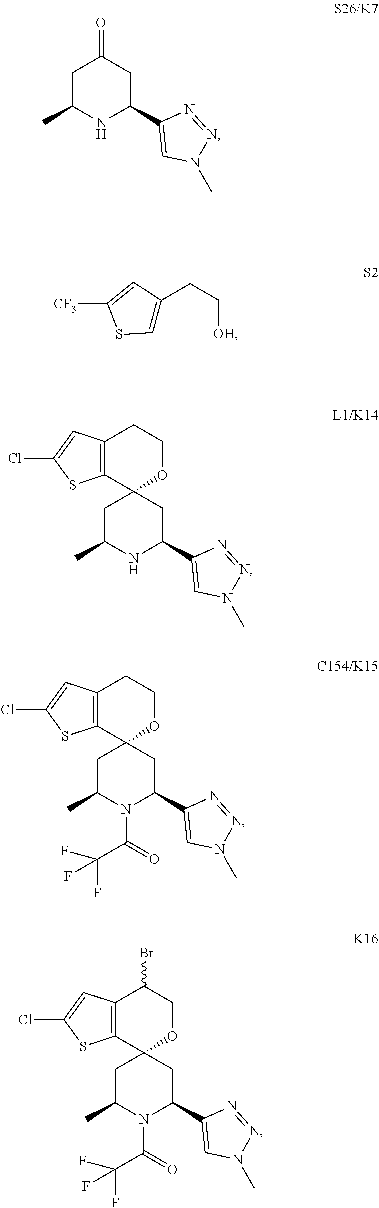

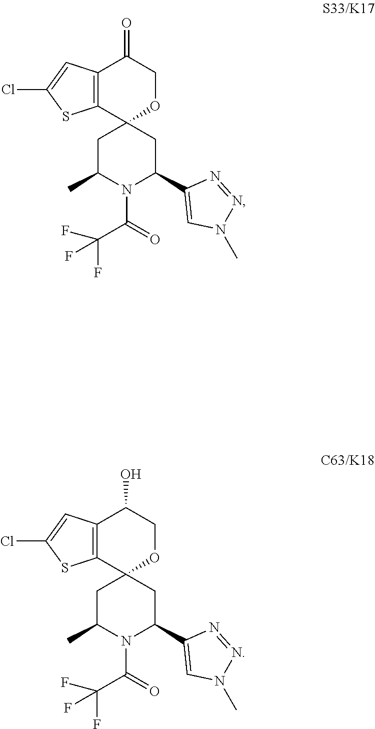 [SMILES: C[C@H]1C[C@@]2(C[C@@H](c3cn(C)nn3)N1C(=O)C(F)(F)F)OCC(=O)c1cc(Cl)sc12 ; C[C@H]1C[C@@]2(C[C@@H](c3cn(C)nn3)N1C(=O)C(F)(F)F)OC[C@@H](O)c1cc(Cl)sc12]

In some embodiments, a compound of the disclosure is selected from:

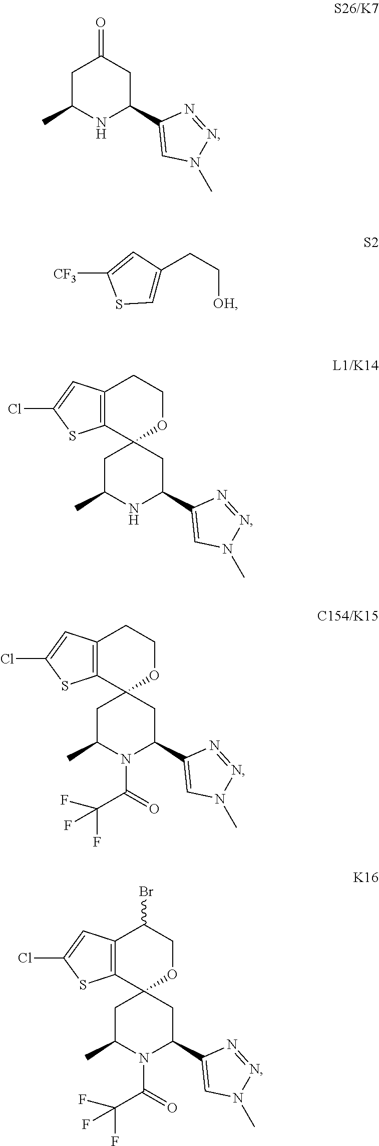

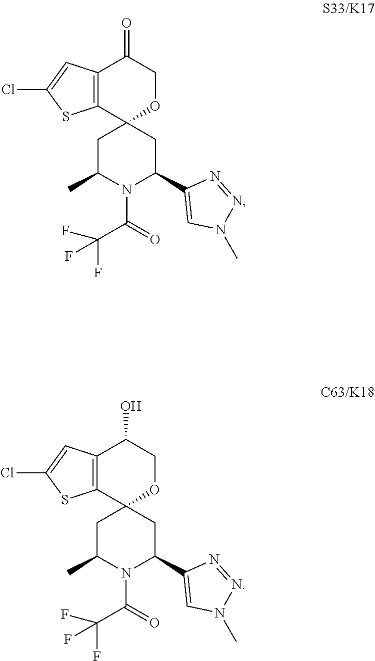 [SMILES: C[C@H]1C[C@@]2(C[C@@H](c3cn(C)nn3)N1C(=O)C(F)(F)F)OCC(=O)c1cc(Cl)sc12 ; C[C@H]1C[C@@]2(C[C@@H](c3cn(C)nn3)N1C(=O)C(F)(F)F)OC[C@@H](O)c1cc(Cl)sc12]

There are several non-limiting advantages to forming Compound II according to Scheme 2 and the embodiments described above. These advantages are even more apparent when manufacturing Compound II on an industrial scale. For example, the crystallization/isolation of Step 1 (Scheme 2) has been improved, resulting in better slurry properties, improved scalability, processability, and throughput for Step 1. The parameters for Step 3 and Step 4 (Scheme 2) have also been improved in several ways, including by changing the amount and addition profile of AIBN, resulting in a significant reduction in the reaction temperature and improved process safety profile. The parameters for Step 5 (Scheme 2) have also been optimized, enabling a significant reduction in the amount of rhodium catalyst used. Finally, the process of Step 6 (Scheme 2) has been developed to allow for isolation of Form C.

In some embodiments, Compound I is prepared according to Scheme 3.

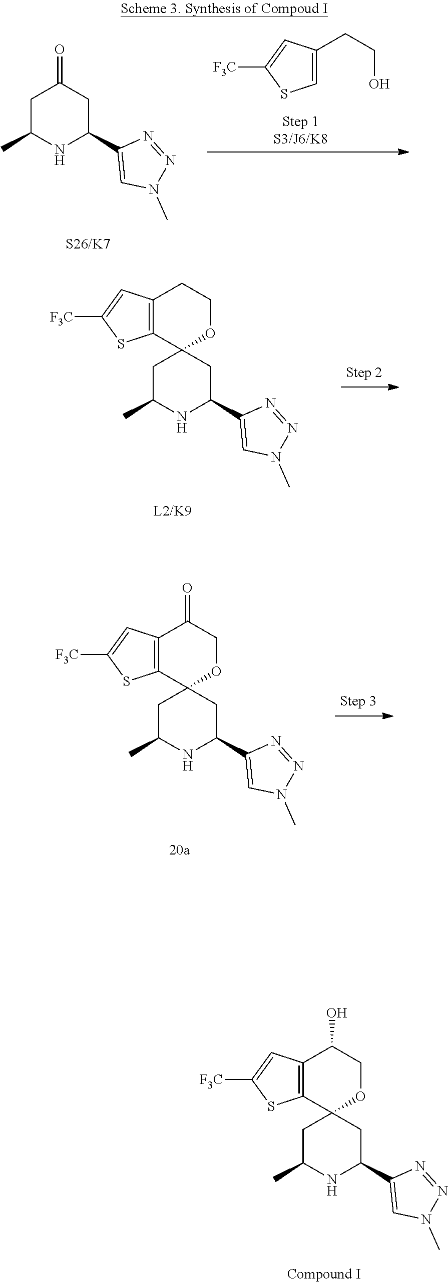

In some embodiments, Compound I is prepared by converting Compound 20a:

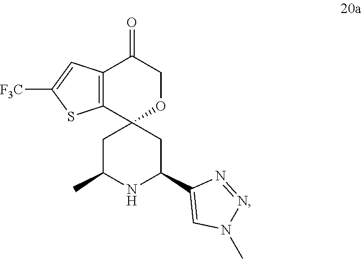 [SMILES: C[C@H]1C[C@@]2(C[C@@H](c3cn(C)nn3)N1)OCC(=O)c1cc(C(F)(F)F)sc12]

into Compound I.

In some embodiments, converting Compound 20a into Compound I is performed in the presence of pentamethylcyclopentadienyl rhodium chloride dimer (RhCl₂Cp*)₂, (R,R)—N-(p-toluenesulfonyl)-1,2-diphenylethylenediamine ((R,R)-TsDPEN), formic acid (HCO₂H), and triethylamine (Et₃N).

In some embodiments, converting Compound 20a into Compound I is performed at −15 to 0° C.

In some embodiments, Compound 20a is prepared by converting Compound L2/K9:

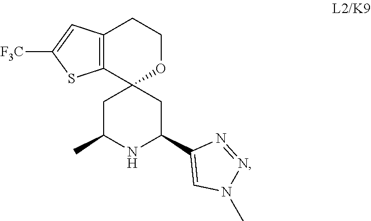 [SMILES: C[C@H]1C[C@@]2(C[C@@H](c3cn(C)nn3)N1)OCCc1cc(C(F)(F)F)sc12]

into Compound 20a.

In some embodiments, converting Compound L2/K9 into Compound 20a is performed in the presence of 2,4,6-triphenylpyrylium tetrafluoroborate, an acid, 460 nm LEDs, and air/N2.

In some embodiments, converting Compound L2/K9 into Compound 20a is performed in the presence of 2,4,6-triphenylpyrylium tetrafluoroborate, methanesulfonic acid (MsOH), 460 nm LEDs, and air/N2.

In some embodiments, converting Compound L2/K9 into Compound 20a is performed in the presence of copper (II) acetate, ammonium persulfate, and water.

In some embodiments, Compound L2/K9 is prepared by reacting Compound S26/K7:

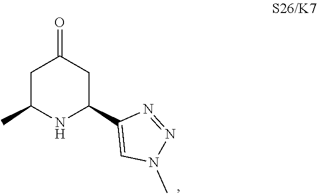

with Compound S3/J6/K8:

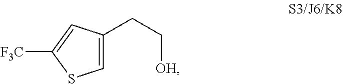 [SMILES: OCCc1csc(C(F)(F)F)c1]

to produce Compound L2/K9.

In some embodiments, reacting Compound S26/K7 with Compound S3/J6/K8 is performed in the presence of methanesulfonic acid (MsOH).

In some embodiments, reacting Compound S26/K7 with Compound S3/J6/K8 is performed at 39° C.

In some embodiments, Compound I is prepared using a compound selected from:

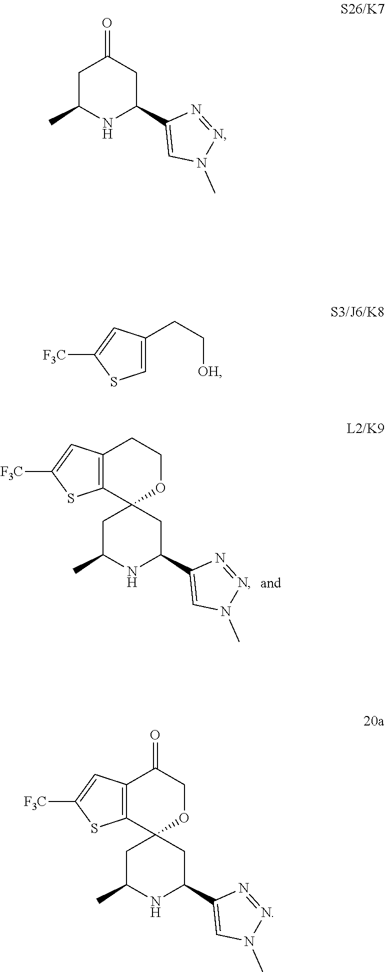 [SMILES: C[C@H]1CC(=O)C[C@@H](c2cn(C)nn2)N1 ; OCCc1csc(C(F)(F)F)c1 ; C[C@H]1C[C@@]2(C[C@@H](c3cn(C)nn3)N1)OCCc1cc(C(F)(F)F)sc12 ; "and" ; C[C@H]1C[C@@]2(C[C@@H](c3cn(C)nn3)N1)OCC(=O)c1cc(C(F)(F)F)sc12]

In some embodiments, a compound of the disclosure is selected from:

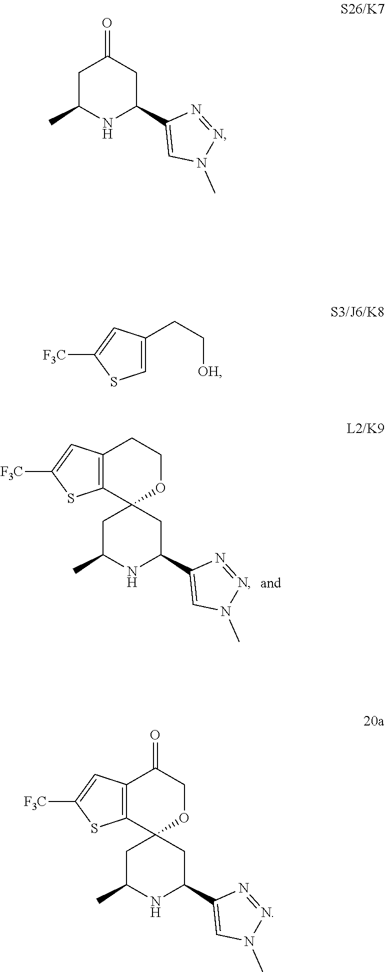 [SMILES: C[C@H]1CC(=O)C[C@@H](c2cn(C)nn2)N1 ; OCCc1csc(C(F)(F)F)c1 ; C[C@H]1C[C@@]2(C[C@@H](c3cn(C)nn3)N1)OCCc1cc(C(F)(F)F)sc12 ; "and" ; C[C@H]1C[C@@]2(C[C@@H](c3cn(C)nn3)N1)OCC(=O)c1cc(C(F)(F)F)sc12]

Preparing Compound I according to Scheme 3 uses a significantly shorter route (three overall steps), which results in a higher yield/throughput.

In some embodiments, Compound II is prepared according to Scheme 4.

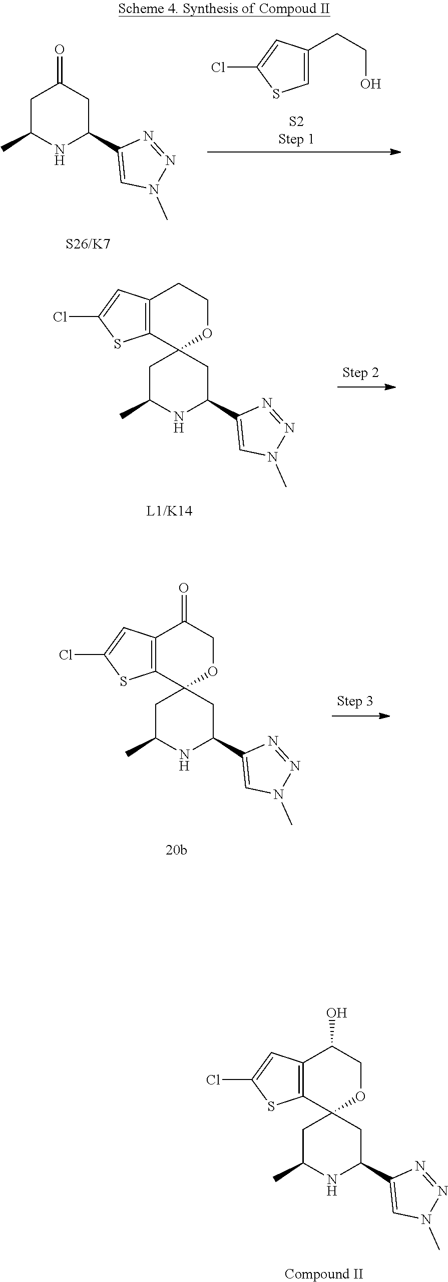

In some embodiments, Compound II is prepared by converting Compound 20b:

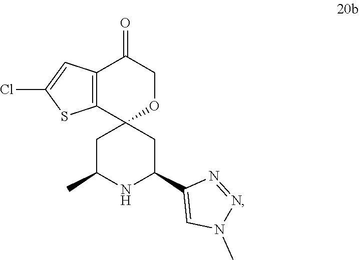 [SMILES: C[C@H]1C[C@@]2(C[C@@H](c3cn(C)nn3)N1)OCC(=O)c1cc(Cl)sc12]

into Compound II.

In some embodiments, converting Compound 20b into Compound II is performed in the presence of pentamethylcyclopentadienyl rhodium chloride dimer (RhCl₂Cp*)₂, (R,R)—N-(p-toluenesulfonyl)-1,2-diphenylethylenediamine ((R,R)-TsDPEN), formic acid (HCO₂H), and triethylamine (Et₃N).

In some embodiments, converting Compound 20b into Compound II is performed at −15 to 0° C.

In some embodiments, Compound 20b is prepared by converting Compound L1/K14:

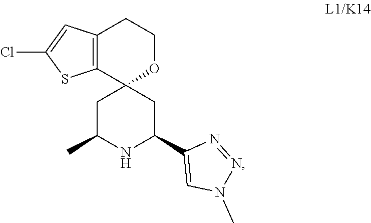 [SMILES: C[C@H]1C[C@@]2(C[C@@H](c3cn(C)nn3)N1)OCCc1cc(Cl)sc12]

into Compound 20b.

In some embodiments, converting Compound L1/K14 into Compound 20b is performed in the presence of 2,4,6-triphenylpyrylium tetrafluoroborate, an acid, 460 nm LEDs, and air/N2.

In some embodiments, converting Compound L1/K14 into Compound 20b is performed in the presence of 2,4,6-triphenylpyrylium tetrafluoroborate, methanesulfonic acid (MsOH), 460 nm LEDs, and air/N2.

In some embodiments, converting Compound L1/K14 into Compound 20b is performed in the presence of copper (II) acetate, ammonium persulfate, and water.

In some embodiments, Compound L1/K14 is prepared by reacting Compound S26/K7:

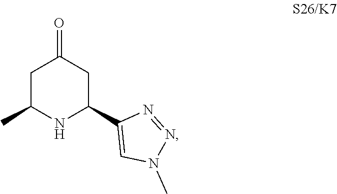

with Compound S2:

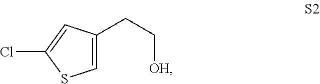

to produce Compound L1/K14.

In some embodiments, reacting Compound S26/K7 with Compound S2 is performed in the presence of methanesulfonic acid (MsOH).

In some embodiments, reacting Compound S26/K7 with Compound S2 is performed at 39° C.

In some embodiments, Compound II is prepared using a compound selected from:

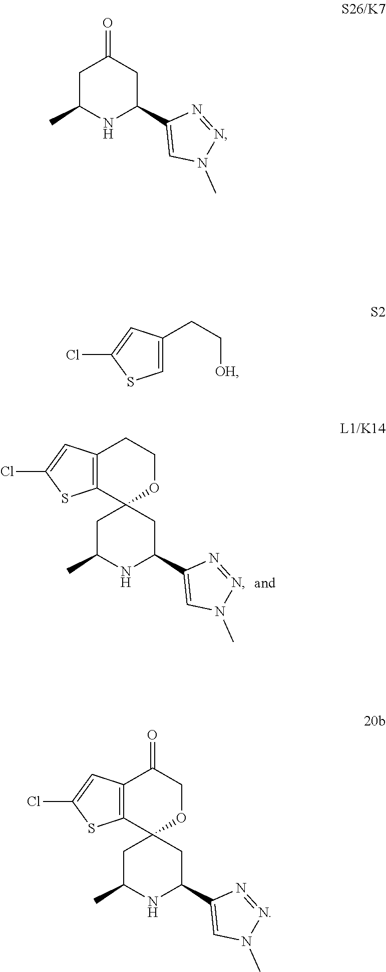 [SMILES: C[C@H]1CC(=O)C[C@@H](c2cn(C)nn2)N1 ; OCCc1csc(Cl)c1 ; C[C@H]1C[C@@]2(C[C@@H](c3cn(C)nn3)N1)OCCc1cc(Cl)sc12 ; "and" ; C[C@H]1C[C@@]2(C[C@@H](c3cn(C)nn3)N1)OCC(=O)c1cc(Cl)sc12]

In some embodiments, a compound of the disclosure is selected from:

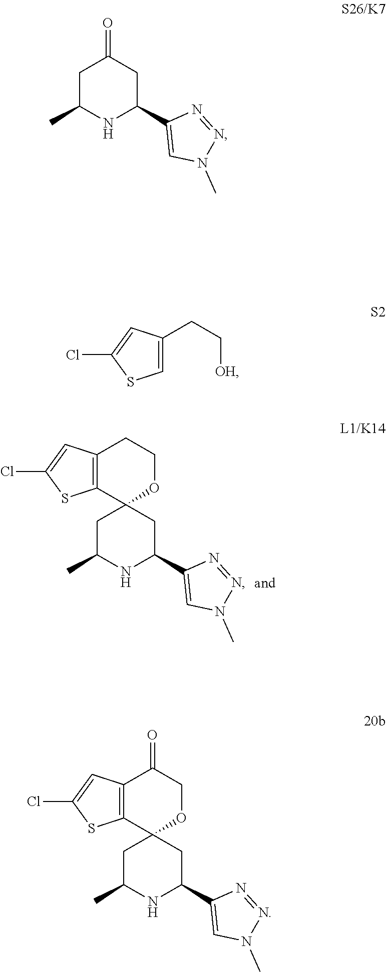 [SMILES: C[C@H]1CC(=O)C[C@@H](c2cn(C)nn2)N1 ; OCCc1csc(Cl)c1 ; C[C@H]1C[C@@]2(C[C@@H](c3cn(C)nn3)N1)OCCc1cc(Cl)sc12 ; "and" ; C[C@H]1C[C@@]2(C[C@@H](c3cn(C)nn3)N1)OCC(=O)c1cc(Cl)sc12]

Preparing Compound II according to Scheme 4 uses a significantly shorter route (three overall steps) and results in a higher yield/throughput.

### Non-Limiting Exemplary Embodiments

Without limitation, some embodiments of this disclosure include:

### 1. Compound I Phosphate Salt Hydrate Form A.

2. Compound I Phosphate Salt Hydrate Form A according to Embodiment 1, characterized by an X-ray powder diffractogram comprising a signal at 8.6±0.2, 19.9±0.2, and/or 28.3±0.2 two-theta.
3. Compound I Phosphate Salt Hydrate Form A according to Embodiment 1, characterized by an X-ray powder diffractogram comprising signals at two or more two-theta values chosen from 8.6±0.2, 19.9±0.2, and 28.3±0.2.
4. Compound I Phosphate Salt Hydrate Form A according to Embodiment 1, characterized by an X-ray powder diffractogram comprising signals at 8.6±0.2, 19.9±0.2, and 28.3±0.2 two-theta.
5. Compound I Phosphate Salt Hydrate Form A according to Embodiment 1, characterized by an X-ray powder diffractogram measured at 25±2° C. and 5% relative humidity (RH) comprising (a) a signal at the following two-theta values: 8.6±0.2, 19.9±0.2, and 28.3±0.2; and (b) a signal at one or more two-theta values chosen from 17.2±0.2, 20.4±0.2, and 22.8±0.2.
6. Compound I Phosphate Salt Hydrate Form A according to Embodiment 1, characterized by an X-ray powder diffractogram measured at 25±2° C. and 5% relative humidity (RH) comprising signals at 8.6±0.2, 17.2±0.2, 19.9±0.2, 20.4±0.2, 22.8±0.2, and 28.3±0.2.
7. Compound I Phosphate Salt Hydrate Form A according to Embodiment 1, characterized by an X-ray powder diffractogram measured at 25±2° C. and 5% relative humidity (RH) comprising (a) a signal at the following two-theta values: 8.6±0.2, 19.9±0.2, and 28.3±0.2; and (b) a signal at one or more two-theta values chosen from 17.2±0.2, 20.4±0.2, 21.1±0.2, 21.9±0.2, and 22.8±0.2.
8. Compound I Phosphate Salt Hydrate Form A according to Embodiment 1, characterized by an X-ray powder diffractogram measured at 25±2° C. and 5% relative humidity (RH) comprising signals at 8.6±0.2, 17.2±0.2, 19.9±0.2, 20.4±0.2, 21.1±0.2, 21.9±0.2, 22.8±0.2, and 28.3±0.2 two-theta.
9. Compound I Phosphate Salt Hydrate Form A according to Embodiment 1, characterized by an X-ray powder diffractogram measured at 25±2° C. and 5% relative humidity (RH) substantially similar to that in FIG. 6.
10. Compound I Phosphate Salt Hydrate Form A according to any one of Embodiments 1-9, characterized by an X-ray powder diffractogram measured at 25±2° C. and 40% relative humidity (RH) comprising (a) a signal at the following two-theta values: 8.6±0.2, 19.9±0.2, and 28.3±0.2; and (b) a signal at one or more two-theta values chosen from 20.4±0.2, 21.0±0.2, and 22.8±0.2.
11. Compound I Phosphate Salt Hydrate Form A according to any one of Embodiments 1-9, characterized by an X-ray powder diffractogram measured at 25±2° C. and 40% relative humidity (RH) comprising signals at 8.6±0.2, 19.9±0.2, 20.4±0.2, 21.0±0.2, 22.8±0.2, and 28.3±0.2.
12. Compound I Phosphate Salt Hydrate Form A according to any one of Embodiments 1-9, characterized by an X-ray powder diffractogram measured at 25±2° C. and 40% relative humidity (RH) comprising (a) a signal at the following two-theta values: 8.6±0.2, 19.9±0.2, and 28.3±0.2; and (b) a signal at one or more two-theta values chosen from 17.2±0.2, 20.4±0.2, 21.0±0.2, 22.8±0.2, and 27.8±0.2.
13. Compound I Phosphate Salt Hydrate Form A according to any one of Embodiments 1-9, characterized by an X-ray powder diffractogram measured at 25±2° C. and 40% relative humidity (RH) comprising signals at 8.6±0.2, 17.2±0.2, 19.9±0.2, 20.4±0.2, 21.0±0.2, 22.8±0.2, 27.8±0.2, and 28.3±0.2 two-theta.
14. Compound I Phosphate Salt Hydrate Form A according to any one of Embodiments 1-9, characterized by an X-ray powder diffractogram measured at 25±2° C. and 40% relative humidity (RH) substantially similar to that in FIG. 5.
15. Compound I Phosphate Salt Hydrate Form A according to any one of Embodiments 1-14, characterized by an X-ray powder diffractogram measured at 25±2° C. and 90% relative humidity (RH) comprising (a) a signal at the following two-theta values: 8.6±0.2, 19.9±0.2, and 28.3±0.2; and (b) a signal at one or more two-theta values chosen from 20.4±0.2, 21.0±0.2, and 27.8±0.2.
16. Compound I Phosphate Salt Hydrate Form A according to any one of Embodiments 1-14, characterized by an X-ray powder diffractogram measured at 25±2° C. and 90% relative humidity (RH) comprising signals at 8.6±0.2, 19.9±0.2, 20.4±0.2, 21.0±0.2, 27.8±0.2, and 28.3±0.2.
17. Compound I Phosphate Salt Hydrate Form A according to any one of Embodiments 1-14, characterized by an X-ray powder diffractogram measured at 25±2° C. and 90% relative humidity (RH) comprising (a) a signal at the following two-theta values: 8.6±0.2, 19.9±0.2, and 28.3±0.2; and (b) a signal at one or more two-theta values chosen from 17.2±0.2, 20.4±0.2, 21.0±0.2, 22.8±0.2, and 27.8±0.2.
18. Compound I Phosphate Salt Hydrate Form A according to any one of Embodiments 1-14, characterized by an X-ray powder diffractogram measured at 25±2° C. and 90% relative humidity (RH) comprising signals at 8.6±0.2, 17.2±0.2, 19.9±0.2, 20.4±0.2, 21.0±0.2, 22.8±0.2, 27.8±0.2, and 28.3±0.2 two-theta.
19. Compound I Phosphate Salt Hydrate Form A according to any one of Embodiments 1-14, characterized by an X-ray powder diffractogram measured at 25±2° C. and 90% relative humidity (RH) substantially similar to that in FIG. 6.
20. Compound I Phosphate Salt Hydrate Form A according to any one of Embodiments 1-19, characterized by a ¹³C NMR spectrum measured at 43% relative humidity (RH) comprising one or more signals chosen from 16.0±0.2 ppm, 38.4±0.2 ppm, 128.6±0.2 ppm, 139.3±0.2 ppm, and 141.7±0.2 ppm.
21. Compound I Phosphate Salt Hydrate Form A according to any one of Embodiments 1-19, characterized by a ¹³C NMR spectrum measured at 43% relative humidity (RH) comprising one or more signals chosen from 16.0±0.2 ppm, 36.7±0.2 ppm, 38.4±0.2 ppm, 126.6±0.2 ppm, 128.6±0.2 ppm, 129.4±0.2 ppm, 139.3±0.2 ppm, 141.7±0.2 ppm, 144±0.2 ppm, and 145.8±0.2 ppm.
22. Compound I Phosphate Salt Hydrate Form A according to any one of Embodiments 1-19, characterized by a ¹³C NMR spectrum measured at 43% relative humidity (RH) comprising signals at 16.0±0.2 ppm, 38.4±0.2 ppm, 128.6±0.2 ppm, 139.3±0.2 ppm, and 141.7±0.2 ppm.
23. Compound I Phosphate Salt Hydrate Form A according to any one of Embodiments 1-19, characterized by a ¹³C NMR spectrum measured at 43% relative humidity (RH) comprising signals at 16.0±0.2 ppm, 36.7±0.2 ppm, 38.4±0.2 ppm, 126.6±0.2 ppm, 128.6±0.2 ppm, 129.4±0.2 ppm, 139.3±0.2 ppm, 141.7±0.2 ppm, 144±0.2 ppm, and 145.8±0.2 ppm.
24. Compound I Phosphate Salt Hydrate Form A according to any one of Embodiments 1-19, characterized by a ¹³C NMR spectrum measured at 43% relative humidity (RH) substantially similar to that in FIG. 7.
25. Compound I Phosphate Salt Hydrate Form A according to any one of Embodiments 1-24, characterized by a ¹⁹F NMR spectrum measured at 43% relative humidity (RH) comprising one or more signals chosen from −57.4±0.2 ppm and −53.8±0.2 ppm.
26. Compound I Phosphate Salt Hydrate Form A according to any one of Embodiments 1-24, characterized by a ¹⁹F NMR spectrum measured at 43% relative humidity (RH) comprising signals at −57.4±0.2 ppm and −53.8±0.2 ppm.
27. Compound I Phosphate Salt Hydrate Form A according to any one of Embodiments 1-24, characterized by a ¹⁹F NMR spectrum measured at 43% relative humidity (RH) substantially similar to that in FIG. 8.
28. Compound I Phosphate Salt Hydrate Form A according to any one of Embodiments 1-27, characterized by a ³¹P NMR spectrum measured at 43% relative humidity (RH) comprising one or more signals chosen from 2.6±0.2 ppm and 4.2±0.2 ppm.
29. Compound I Phosphate Salt Hydrate Form A according to any one of Embodiments 1-27, characterized by a ³¹P NMR spectrum measured at 43% relative humidity (RH) comprising signals at 2.6±0.2 ppm and 4.2±0.2 ppm.
30. Compound I Phosphate Salt Hydrate Form A according to any one of Embodiments 1-27, characterized by a ³¹P NMR spectrum measured at 43% relative humidity (RH) substantially similar to that in FIG. 10.
31. Compound I Phosphate Salt Hydrate Form A according to any one of Embodiments 1-30, characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 8.9 ± 0.1 Å | α | 90° |
| b | 10.5 ± 0.1 Å | β | 90° |
| c | 45.0 ± 0.1 Å | γ | 90°. |

32. A pharmaceutical composition comprising Compound I Phosphate Salt Hydrate Form A according to any one of Embodiments 1-31 and a pharmaceutically acceptable carrier.
33. A method of treating an APOL1 mediated disease comprising administering to a patient in need thereof Compound I Phosphate Salt Hydrate Form A according to any one of Embodiments 1-31 or a pharmaceutical composition according to Embodiment 32.
34. The method according to Embodiment 33, wherein the APOL1 mediated disease is an APOL1 mediated kidney disease.
35. The method according to Embodiment 34, wherein the APOL1 mediated kidney disease is chosen from ESKD, NDKD, FSGS, HIV-associated nephropathy, arterionephrosclerosis, lupus nephritis, microalbuminuria, and chronic kidney disease.
36. The method according to Embodiment 34 or 35, wherein the APOL1 mediated kidney disease is FSGS or NDKD.
37. The method according to any one of Embodiments 34-36, wherein the APOL1 mediated kidney disease is associated with APOL1 genetic alleles chosen from homozygous G1: S342G:I384M and homozygous G2: N388del:Y389del.
38. The method according to any one of Embodiments 34-36, wherein the APOL1 mediated kidney disease is associated with compound heterozygous G1: S342G:I384M and G2: N388del:Y389del APOL1 genetic alleles.
39. The method according to Embodiment 33, wherein the APOL1 mediated disease is cancer.
40. The method according to Embodiment 33 or 39, wherein the APOL1 mediated disease is pancreatic cancer.
41. A method of inhibiting APOL1 activity comprising contacting said APOL1 with Compound I Phosphate Salt Hydrate Form A according to any one of Embodiments 1-31 or a pharmaceutical composition according to Embodiment 32.
42. The method according to Embodiment 41, wherein the APOL1 is associated with APOL1 genetic alleles chosen from homozygous G1: S342G:I384M and homozygous G2: N388del:Y389del.
43. The method according to Embodiment 41, wherein the APOL1 is associated with compound heterozygous G1: S342G:I384M and G2: N388del:Y389del APOL1 genetic alleles.
44. Use of Compound I Phosphate Salt Hydrate Form A according to any one of Embodiments 1-31 in the manufacture of a medicament for treating APOL1 mediated disease (e.g., APOL1 mediated kidney disease).
45. Compound I Phosphate Salt Hydrate Form A according to any one of Embodiments 1-31 for use in treating APOL1 mediated disease (e.g., APOL1 mediated kidney disease).
46. A method of preparing Compound I Phosphate Salt Hydrate Form A comprising drying Compound I Phosphate Salt Methanol Solvate at about 50° C.
47. A method of preparing Compound I Phosphate Salt Hydrate Form A comprising:

charging Compound I free form Monohydrate and MEK to a reactor;

agitating the reactor;

adding water to the reactor and further agitating;

seeding the reactor with Compound I Phosphate Salt Hydrate Form A;

slowly adding a phosphoric acid solution to the reactor; and

agitating the reactor at about 20° C.

48. A method of preparing Compound I Phosphate Salt Hydrate Form A comprising:

charging Compound I Monohydrate and MEK to a reactor;

agitating the reactor;

adding water to the reactor and further agitating;

slowly adding a phosphoric acid solution to the reactor; and

agitating the reactor at about 20° C.

49. Compound I free form Monohydrate.
50. Compound I free form Monohydrate according to Embodiment 49, characterized by an X-ray powder diffractogram comprising a signal at 8.7±0.2, 12.8±0.2, 16.7±0.2, and/or 21.7±0.2 two-theta.
51. Compound I free form Monohydrate according to Embodiment 49, characterized by an X-ray powder diffractogram comprising at two or more two-theta values chosen from 8.7±0.2, 12.8±0.2, 16.7±0.2, and 21.7±0.2.
52. Compound I free form Monohydrate according to Embodiment 49, characterized by an X-ray powder diffractogram comprising signals at 8.7±0.2, 12.8±0.2, 16.7±0.2, and 21.7±0.2 two-theta.
53. Compound I free form Monohydrate according to Embodiment 49, characterized by an X-ray powder diffractogram comprising (a) a signal at the following two-theta values: 8.7±0.2, 12.8±0.2, 16.7±0.2, and 21.7±0.2; and (b) a signal at one or more two-theta values chosen from 13.8±0.2, 19.8±0.2, and 25.8±0.2.
54. Compound I free form Monohydrate according to Embodiment 49, characterized by an X-ray powder diffractogram comprising signals at 8.7±0.2, 12.8±0.2, 13.8±0.2, 16.7±0.2, 19.8±0.2, 21.7±0.2, and 25.8±0.2 two-theta.
55. Compound I free form Monohydrate according to Embodiment 49, characterized by an X-ray powder diffractogram comprising (a) a signal at the following two-theta values: 8.7±0.2, 12.8±0.2, 16.7±0.2, and 21.7±0.2; and (b) a signal at one or more two-theta values chosen from 13.8±0.2, 15.5±0.2, 19.8±0.2, 24.3±0.2, and 25.8±0.2.
56. Compound I free form Monohydrate according to Embodiment 49, characterized by an X-ray powder diffractogram comprising signals at 8.7±0.2, 12.8±0.2, 13.8±0.2, 15.5±0.2, 16.7±0.2, 19.8±0.2, 21.7±0.2, 24.3±0.2, and 25.8±0.2 two-theta.
57. Compound I free form Monohydrate according to Embodiment 49, characterized by an X-ray powder diffractogram substantially similar to that in FIG. 14.
58. Compound I free form Monohydrate according to any one of Embodiments 49-57, characterized by a ¹³C NMR spectrum measured at 43% relative humidity (RH) comprising one or more signals chosen from 24.9±0.2 ppm, 35.1±0.2 ppm, 39.3±0.2 ppm, 135.3±0.2 ppm, and 149.6±0.2 ppm.
59. Compound I free form Monohydrate according to any one of Embodiments 49-57, characterized by a ¹³C NMR spectrum measured at 43% relative humidity (RH) comprising one or more signals chosen from 24.9±0.2 ppm, 35.1±0.2 ppm, 39.3±0.2 ppm, 126.2±0.2 ppm, 127.7±0.2 ppm, 129.6±0.2 ppm, 135.3±0.2 ppm, 149.4±0.2 ppm, and 149.6±0.2 ppm.
60. Compound I free form Monohydrate according to any one of Embodiments 49-57, characterized by a ¹³C NMR spectrum measured at 43% relative humidity (RH) comprising signals at 24.9±0.2 ppm, 35.1±0.2 ppm, 39.3±0.2 ppm, 135.3±0.2 ppm, and 149.6±0.2 ppm.
61. Compound I free form Monohydrate according to any one of Embodiments 49-57, characterized by a ¹³C NMR spectrum measured at 43% relative humidity (RH) comprising signals at 24.9±0.2 ppm, 35.1±0.2 ppm, 39.3±0.2 ppm, 126.2±0.2 ppm, 127.7±0.2 ppm, 129.6±0.2 ppm, 135.3±0.2 ppm, 149.4±0.2 ppm, and 149.6±0.2 ppm.
62. Compound I free form Monohydrate according to any one of Embodiments 49-57, characterized by a ¹³C NMR spectrum measured at 43% relative humidity (RH) substantially similar to that in FIG. 15.
63. Compound I free form Monohydrate according to any one of Embodiments 49-57, characterized by a ¹³C NMR spectrum measured after dehydration (80° C. in rotor overnight (2×), 80° C. weekend incubation with P₂O₅) comprising one or more signals chosen from 25.6±0.2 ppm, 35.8±0.2 ppm, 36.8±0.2 ppm, 135.3±0.2 ppm, and 150±0.2 ppm.
64. Compound I free form Monohydrate according to any one of Embodiments 49-57, characterized by a ¹³C NMR spectrum measured after dehydration (80° C. in rotor overnight (2×), 80° C. weekend incubation with P₂O₅) comprising one or more signals chosen from 25.6±0.2 ppm, 35.8±0.2 ppm, 36.8±0.2 ppm, 126.6±0.2 ppm, 127.2±0.2 ppm, 129.6±0.2 ppm, 135.3±0.2 ppm, 150±0.2 ppm, and 150.9±0.2 ppm.
65. Compound I free form Monohydrate according to any one of Embodiments 49-57, characterized by a ¹³C NMR spectrum measured after dehydration (80° C. in rotor overnight (2×), 80° C. weekend incubation with P₂O₅) comprising signals at 25.6±0.2 ppm, 35.8±0.2 ppm, 36.8±0.2 ppm, 135.3±0.2 ppm, and 150±0.2 ppm.
66. Compound I free form Monohydrate according to any one of Embodiments 49-57, characterized by a ¹³C NMR spectrum measured after dehydration (80° C. in rotor overnight (2×), 80° C. weekend incubation with P₂O₅) comprising signals at 25.6±0.2 ppm, 35.8±0.2 ppm, 36.8±0.2 ppm, 126.6±0.2 ppm, 127.2±0.2 ppm, 129.6±0.2 ppm, 135.3±0.2 ppm, 150±0.2 ppm, and 150.9±0.2 ppm.
67. Compound I free form Monohydrate according to any one of Embodiments 49-57, characterized by a ¹³C NMR spectrum measured after dehydration (80° C. in rotor overnight (2×), 80° C. weekend incubation with P₂O₅) substantially similar to that in FIG. 16.
68. Compound I free form Monohydrate according to any one of Embodiments 49-67, characterized by a ¹⁹F NMR spectrum measured at 43% relative humidity (RH) comprising a signal at −55.8±0.2 ppm.
69. Compound I free form Monohydrate according to any one of Embodiments 49-67, characterized by a ¹⁹F NMR spectrum measured at 43% relative humidity (RH) substantially similar to that in FIG. 17.
70. Compound I free form Monohydrate according to any one of Embodiments 49-67, characterized by a ¹⁹F NMR spectrum measured after dehydration (80° C. in rotor overnight (2×), 80° C. weekend incubation with P₂O₅) comprising a signal at −55.5±0.2 ppm.
71. Compound I free form Monohydrate according to any one of Embodiments 49-67, characterized by a ¹⁹F NMR spectrum measured after dehydration (80° C. in rotor overnight (2×), 80° C. weekend incubation with P₂O₅) substantially similar to that in FIG. 18.
72. Compound I free form Monohydrate according to any one of Embodiments 49-71, characterized by a tetragonal crystal system, a P43 space group, and unit cell dimensions measured at 100 K on a Bruker diffractometer equipped Cu Kα radiation (λ=1.54178 Å) of:

| | | | |
|---|---|---|---|
| a | 14.2 ± 0.1 Å | α | 90° |
| b | 14.2 ± 0.1 Å | β | 90° |
| c | 9.3 ± 0.1 Å | γ | 90°. |

73. Compound I free form Monohydrate according to any one of Embodiments 49-72, characterized by a tetragonal crystal system, a P43 space group, and unit cell dimensions measured at 100 K on a Bruker diffractometer equipped Cu Kα radiation (λ=1.54178 Å) after drying at 325 K under dry nitrogen for 1 hour of:

| | | | |
|---|---|---|---|
| a | 14.3 ± 0.1 Å | α | 90° |
| b | 14.3 ± 0.1 Å | β | 90° |
| c | 9.2 ± 0.1 Å | γ | 90°. |

74. A pharmaceutical composition comprising Compound I free form Monohydrate according to any one of Embodiments 49-73 and a pharmaceutically acceptable carrier.
75. A method of treating an APOL1 mediated disease comprising administering to a patient in need thereof Compound I free form Monohydrate according to any one of Embodiments 49-73 or a pharmaceutical composition according to Embodiment 74.
76. The method according to Embodiment 75, wherein the APOL1 mediated disease is an APOL1 mediated kidney disease.
77. The method according to Embodiment 76, wherein the APOL1 mediated kidney disease is chosen from ESKD, NDKD, FSGS, HIV-associated nephropathy, arterionephrosclerosis, lupus nephritis, microalbuminuria, and chronic kidney disease.
78. The method according to Embodiment 76 or 77, wherein the APOL1 mediated kidney disease is FSGS or NDKD.
79. The method according to any one of Embodiments 76-78, wherein the APOL1 mediated kidney disease is associated with APOL1 genetic alleles chosen from homozygous G1: S342G:I384M and homozygous G2: N388del:Y389del.
80. The method according to any one of Embodiments 76-78, wherein the APOL1 mediated kidney disease is associated with compound heterozygous G1: S342G:I384M and G2: N388del:Y389del APOL1 genetic alleles.
81. The method according to Embodiment 75, wherein the APOL1 mediated disease is cancer.
82. The method according to Embodiment 75 or 81, wherein the APOL1 mediated disease is pancreatic cancer.
83. A method of inhibiting APOL1 activity comprising contacting said APOL1 with Compound I free form Monohydrate according to any one of Embodiments 49-73 or a pharmaceutical composition according to Embodiment 74.
84. The method according to Embodiment 83, wherein the APOL1 is associated with APOL1 genetic alleles chosen from homozygous G1: S342G:I384M and homozygous G2: N388del:Y389del.
85. The method according to Embodiment 83, wherein the APOL1 is associated with compound heterozygous G1: S342G:I384M and G2: N388del:Y389del APOL1 genetic alleles.
86. Use of Compound I free form Monohydrate according to any one of Embodiments 49-73 in the manufacture of a medicament for treating APOL1 mediated disease (e.g., APOL1 mediated kidney disease).
87. Compound I free form Monohydrate according to any one of Embodiments 49-73 for use in treating APOL1 mediated disease (e.g., APOL1 mediated kidney disease).
88. A method of preparing Compound I free form Monohydrate comprising:

adding amorphous Compound I to saline to create a solution;

incubating the solution at ambient temperature;

filtering the solution to obtain a solid material; and

drying the solid material.

### 89. Compound I Phosphate Salt Methanol Solvate.

90. Compound I Phosphate Salt Methanol Solvate according to Embodiment 89, characterized by an X-ray powder diffractogram comprising a signal at 12.7±0.2, 14.8±0.2, and/or 20.7±0.2 two-theta.
91. Compound I Phosphate Salt Methanol Solvate according to Embodiment 89, characterized by an X-ray powder diffractogram comprising at two or more two-theta values chosen from 12.7±0.2, 14.8±0.2, and 20.7±0.2.
92. Compound I Phosphate Salt Methanol Solvate according to Embodiment 89, characterized by an X-ray powder diffractogram comprising signals at 12.7±0.2, 14.8±0.2, and 20.7±0.2 two-theta.
93. Compound I Phosphate Salt Methanol Solvate according to Embodiment 89, characterized by an X-ray powder diffractogram comprising (a) a signal at the following two-theta values: 12.7±0.2, 14.8±0.2, and 20.7±0.2; and (b) a signal at one or more two-theta values chosen from 8.5±0.2, 15.8±0.2, and 19.5±0.2.
94. Compound I Phosphate Salt Methanol Solvate according to Embodiment 89, characterized by an X-ray powder diffractogram comprising signals at 8.5±0.2, 12.7±0.2, 14.8±0.2, 15.8±0.2, 19.5±0.2, and 20.7±0.2 two-theta.
95. Compound I Phosphate Salt Methanol Solvate according to Embodiment 89, characterized by an X-ray powder diffractogram comprising (a) a signal at the following two-theta values: 12.7±0.2, 14.8±0.2, and 20.7±0.2; and (b) a signal at one or more two-theta values chosen from 8.5±0.2, 13.9±0.2, 15.8±0.2, 18.7±0.2, and 19.5±0.2.
96. Compound I Phosphate Salt Methanol Solvate according to Embodiment 89, characterized by an X-ray powder diffractogram comprising signals at 8.5±0.2, 12.7±0.2, 13.9±0.2, 14.8±0.2, 15.8±0.2, 18.7±0.2, 19.5±0.2, and 20.7±0.2 two-theta.
97. Compound I Phosphate Salt Methanol Solvate according to Embodiment 89, characterized by an X-ray powder diffractogram substantially similar to that in FIG. 1.
98. Compound I Phosphate Salt Methanol Solvate according to any one of Embodiments 89-97, characterized by a ¹³C NMR spectrum comprising one or more signals chosen from 15.7±0.2 ppm, 17.7±0.2 ppm, 38.9±0.2 ppm, 129.4±0.2 ppm, and 140.6±0.2 ppm.
99. Compound I Phosphate Salt Methanol Solvate according to any one of Embodiments 89-97, characterized by a ¹³C NMR spectrum comprising one or more signals chosen from 15.7±0.2 ppm, 17.7±0.2 ppm, 36.8±0.2 ppm, 37.7±0.2 ppm, 38.9±0.2 ppm, 127.9±0.2 ppm, 128.5±0.2 ppm, 129.4±0.2 ppm, 139.5±0.2 ppm, and 140.6±0.2 ppm.
100. Compound I Phosphate Salt Methanol Solvate according to any one of Embodiments 89-97, characterized by a ¹³C NMR spectrum comprising signals at 15.7±0.2 ppm, 17.7±0.2 ppm, 38.9±0.2 ppm, 129.4±0.2 ppm, and 140.6±0.2 ppm.
101. Compound I Phosphate Salt Methanol Solvate according to any one of Embodiments 89-97, characterized by a ¹³C NMR spectrum comprising signals at 15.7±0.2 ppm, 17.7±0.2 ppm, 36.8±0.2 ppm, 37.7±0.2 ppm, 38.9±0.2 ppm, 127.9±0.2 ppm, 128.5±0.2 ppm, 129.4±0.2 ppm, 139.5±0.2 ppm, and 140.6±0.2 ppm.
102. Compound I Phosphate Salt Methanol Solvate according to any one of Embodiments 89-97, characterized by a ¹³C NMR spectrum substantially similar to that in FIG. 2.
103. Compound I Phosphate Salt Methanol Solvate according to any one of Embodiments 89-102, characterized by a ¹⁹F NMR spectrum comprising one or more signals chosen from −57.7±0.2 ppm and −54.7±0.2 ppm.
104. Compound I Phosphate Salt Methanol Solvate according to any one of Embodiments 89-102, characterized by a ¹⁹F NMR spectrum comprising signals at −57.7±0.2 ppm and −54.7±0.2 ppm.
105. Compound I Phosphate Salt Methanol Solvate according to any one of Embodiments 89-102, characterized by a ¹⁹F NMR spectrum substantially similar to that in FIG. 3.
106. Compound I Phosphate Salt Methanol Solvate according to any one of Embodiments 89-105, characterized by a ³¹P NMR spectrum comprising one or more signals chosen from 1.8±0.2 ppm and 2.5±0.2 ppm.
107. Compound I Phosphate Salt Methanol Solvate according to any one of Embodiments 89-105, characterized by a ³¹P NMR spectrum comprising signals at 1.8±0.2 ppm and 2.5±0.2 ppm.
108. Compound I Phosphate Salt Methanol Solvate according to any one of Embodiments 89-105, characterized by a ³¹P NMR spectrum substantially similar to that in FIG. 4.
109. Compound I Phosphate Salt Methanol Solvate according to any one of Embodiments 89-108, characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 9.4 ± 0.1 Å | α | 90° |
| b | 10.5 ± 0.1 Å | β | 90° |
| c | 44.6 ± 0.1 Å | γ | 90°. |

110. A pharmaceutical composition comprising Compound I Phosphate Salt Methanol Solvate according to any one of Embodiments 89-109 and a pharmaceutically acceptable carrier.
111. A method of treating an APOL1 mediated disease comprising administering to a patient in need thereof Compound I Phosphate Salt Methanol Solvate according to any one of Embodiments 89-109 or a pharmaceutical composition according to Embodiment 110.
112. The method according to Embodiment 111, wherein the APOL1 mediated disease is an APOL1 mediated kidney disease.
113. The method according to Embodiment 112, wherein the APOL1 mediated kidney disease is chosen from ESKD, NDKD, FSGS, HIV-associated nephropathy, arterionephrosclerosis, lupus nephritis, microalbuminuria, and chronic kidney disease.
114. The method according to Embodiment 112 or 113, wherein the APOL1 mediated kidney disease is FSGS or NDKD.
115. The method according to any one of Embodiments 112-114, wherein the APOL1 mediated kidney disease is associated with APOL1 genetic alleles chosen from homozygous G1: S342G:I384M and homozygous G2: N388del:Y389del.
116. The method according to any one of Embodiments 112-114, wherein the APOL1 mediated kidney disease is associated with compound heterozygous G1: S342G:I384M and G2: N388del:Y389del APOL1 genetic alleles.
117. The method according to Embodiment 111, wherein the APOL1 mediated disease is cancer.
118. The method according to Embodiment 111 or 117, wherein the APOL1 mediated disease is pancreatic cancer.
119. A method of inhibiting APOL1 activity comprising contacting said APOL1 with Compound I Phosphate Salt Methanol Solvate according to any one of Embodiments 89-109 or a pharmaceutical composition according to Embodiment 110.
120. The method according to Embodiment 119, wherein the APOL1 is associated with APOL1 genetic alleles chosen from homozygous G1: S342G:I384M and homozygous G2: N388del:Y389del.
121. The method according to Embodiment 119, wherein the APOL1 is associated with compound heterozygous G1: S342G:I384M and G2: N388del:Y389del APOL1 genetic alleles.
122. Use of Compound I Phosphate Salt Methanol Solvate according to any one of Embodiments 89-109 in the manufacture of a medicament for treating APOL1 mediated disease (e.g., APOL1 mediated kidney disease).
123. Compound I Phosphate Salt Methanol Solvate according to any one of Embodiments 89-109 for use in treating APOL1 mediated disease (e.g., APOL1 mediated kidney disease).
124. A method of Compound I Phosphate Salt Methanol Solvate comprising:

adding amorphous Compound I to MEK to create a solution;

adding H₃PO₄ to the solution;

incubating the solution at ambient temperature;

filtering the solution to isolate a solid material; and

washing the solid material.

### 125. Compound I Phosphate Salt MEK Solvate.

126. Compound I Phosphate Salt MEK Solvate according to Embodiment 125, characterized by an X-ray powder diffractogram comprising a signal at 8.6±0.2, 15.4±0.2, and/or 20.1±0.2 two-theta.
127. Compound I Phosphate Salt MEK Solvate according to Embodiment 125, characterized by an X-ray powder diffractogram comprising at two or more two-theta values chosen from 8.6±0.2, 15.4±0.2, and 20.1±0.2.
128. Compound I Phosphate Salt MEK Solvate according to Embodiment 125, characterized by an X-ray powder diffractogram comprising signals at 8.6±0.2, 15.4±0.2, and 20.1±0.2 two-theta.
129. Compound I Phosphate Salt MEK Solvate according to Embodiment 125, characterized by an X-ray powder diffractogram comprising (a) a signal at the following two-theta values: 8.6±0.2, 15.4±0.2, and 20.1±0.2; and (b) a signal at one or more two-theta values chosen from 15.7±0.2, 18.2±0.2, and 19.4±0.2.
130. Compound I Phosphate Salt MEK Solvate according to Embodiment 125, characterized by an X-ray powder diffractogram comprising signals at 8.6±0.2, 15.4±0.2, 15.7±0.2, 18.2±0.2, 19.4±0.2, and 20.1±0.2 two-theta.
131. Compound I Phosphate Salt MEK Solvate according to Embodiment 125, characterized by an X-ray powder diffractogram comprising (a) a signal at the following two-theta values: 8.6±0.2, 15.4±0.2, and 20.1±0.2; and (b) a signal at one or more two-theta values chosen from 15.7±0.2, 18.2±0.2, 19.4±0.2, 21.7±0.2, and 21.9±0.2.
132. Compound I Phosphate Salt MEK Solvate according to Embodiment 125, characterized by an X-ray powder diffractogram comprising signals at 8.6±0.2, 15.4±0.2, 15.7±0.2, 18.2±0.2, 19.4±0.2, 20.1±0.2, 21.7±0.2, and 21.9±0.2 two-theta.
133. Compound I Phosphate Salt MEK Solvate according to Embodiment 125, characterized by an X-ray powder diffractogram substantially similar to that in FIG. 21.
134. Compound I Phosphate Salt MEK Solvate according to any one of Embodiments 125-133, characterized by a ¹³C NMR spectrum comprising one or more signals chosen from 16.0±0.2 ppm, 37.5±0.2 ppm, 38.4±0.2 ppm, 126.5±0.2 ppm, and 142.0±0.2 ppm.
135. Compound I Phosphate Salt MEK Solvate according to any one of Embodiments 125-133, characterized by a ¹³C NMR spectrum comprising one or more signals chosen from 7.4±0.2 ppm, 16.0±0.2 ppm, 36.8±0.2 ppm, 37.5±0.2 ppm, 38.4±0.2 ppm, 126.5±0.2 ppm, 128.7±0.2 ppm, 129.6±0.2 ppm, 139.4±0.2 ppm, and 142.0±0.2 ppm.
136. Compound I Phosphate Salt MEK Solvate according to any one of Embodiments 125-133, characterized by a ¹³C NMR spectrum comprising signals at 16.0±0.2 ppm, 37.5±0.2 ppm, 38.4±0.2 ppm, 126.5±0.2 ppm, and 142.0±0.2 ppm.
137. Compound I Phosphate Salt MEK Solvate according to any one of Embodiments 125-133, characterized by a ¹³C NMR spectrum comprising signals at 7.4±0.2 ppm, 16.0±0.2 ppm, 36.8±0.2 ppm, 37.5±0.2 ppm, 38.4±0.2 ppm, 126.5±0.2 ppm, 128.7±0.2 ppm, 129.6±0.2 ppm, 139.4±0.2 ppm, and 142.0±0.2 ppm.
138. Compound I Phosphate Salt MEK Solvate according to any one of Embodiments 125-137, characterized by a ¹³C NMR spectrum substantially similar to that in FIG. 22.
139. Compound I Phosphate Salt MEK Solvate according to any one of Embodiments 125-138, characterized by a ¹⁹F NMR spectrum comprising one or more signals chosen from −53.6±0.2 ppm, −55.2±0.2 ppm, and −57.2±0.2 ppm.
140. Compound I Phosphate Salt MEK Solvate according to any one of Embodiments 125-138, characterized by a ¹⁹F NMR spectrum comprising signals at −53.6±0.2 ppm, −55.2±0.2 ppm, and −57.2±0.2 ppm.
141. Compound I Phosphate Salt MEK Solvate according to any one of Embodiments 125-138, characterized by a ¹⁹F NMR spectrum substantially similar to that in FIG. 23.
142. Compound I Phosphate Salt MEK Solvate according to any one of Embodiments 125-141, characterized by a ³¹P NMR spectrum comprising one or more signals chosen from 0.1±0.2 ppm, 2.7±0.2 ppm, and 4.8±0.2 ppm.
143. Compound I Phosphate Salt MEK Solvate according to any one of Embodiments 125-141, characterized by a ³¹P NMR spectrum comprising signals at 0.1±0.2 ppm, 2.7±0.2 ppm, and 4.8±0.2 ppm.
144. A pharmaceutical composition comprising Compound I Phosphate Salt MEK Solvate according to any one of Embodiments 125-143 and a pharmaceutically acceptable carrier.
145. A method of treating an APOL1 mediated disease comprising administering to a patient in need thereof Compound I Phosphate Salt MEK Solvate according to any one of Embodiments 125-143 or a pharmaceutical composition according to Embodiment 144.
146. The method according to Embodiment 145, wherein the APOL1 mediated disease is an APOL1 mediated kidney disease.
147. The method according to Embodiment 146, wherein the APOL1 mediated kidney disease is chosen from ESKD, NDKD, FSGS, HIV-associated nephropathy, arterionephrosclerosis, lupus nephritis, microalbuminuria, and chronic kidney disease.
148. The method according to Embodiment 146 or 147, wherein the APOL1 mediated kidney disease is FSGS or NDKD.
149. The method according to any one of Embodiments 146-148, wherein the APOL1 mediated kidney disease is associated with APOL1 genetic alleles chosen from homozygous G1: S342G:I384M and homozygous G2: N388del:Y389del.
150. The method according to any one of Embodiments 146-148, wherein the APOL1 mediated kidney disease is associated with compound heterozygous G1: S342G:I384M and G2: N388del:Y389del APOL1 genetic alleles.
151. The method according to Embodiment 145, wherein the APOL1 mediated disease is cancer.
152. The method according to Embodiment 145 or 151, wherein the APOL1 mediated disease is pancreatic cancer.
153. A method of inhibiting APOL1 activity comprising contacting said APOL1 with Compound I Phosphate Salt MEK Solvate according to any one of Embodiments 125-143 or a pharmaceutical composition according to Embodiment 144.
154. The method according to Embodiment 153, wherein the APOL1 is associated with APOL1 genetic alleles chosen from homozygous G1: S342G:I384M and homozygous G2: N388del:Y389del.
155. The method according to Embodiment 153, wherein the APOL1 is associated with compound heterozygous G1: S342G:I384M and G2: N388del:Y389del APOL1 genetic alleles.
156. Use of Compound I Phosphate Salt MEK Solvate according to any one of Embodiments 125-143 in the manufacture of a medicament for treating APOL1 mediated disease (e.g., APOL1 mediated kidney disease).
157. Compound I Phosphate Salt MEK Solvate according to any one of Embodiments 125-143 for use in treating APOL1 mediated disease (e.g., APOL1 mediated kidney disease).
158. A method of preparing Compound I Phosphate Salt MEK Solvate comprising:
adding Compound I Phosphate Salt Hydrate Form A to MEK and mixing to form a slurry;
incubating the slurry at a reduced temperature to obtain a solid material; and
centrifuging the solid material.

### 159. Compound II Phosphate Salt Hemihydrate Form A.

160. Compound II Phosphate Salt Hemihydrate Form A according to Embodiment 159, characterized by an X-ray powder diffractogram comprising a signal at 9.1±0.2, 16.7±0.2, and/or 18.7±0.2 two-theta.
161. Compound II Phosphate Salt Hemihydrate Form A according to Embodiment 159, characterized by an X-ray powder diffractogram comprising at two or more two-theta values chosen from 9.1±0.2, 16.7±0.2, and 18.7±0.2.
162. Compound II Phosphate Salt Hemihydrate Form A according to Embodiment 159, characterized by an X-ray powder diffractogram comprising signals at 9.1±0.2, 16.7±0.2, and 18.7±0.2 two-theta.
163. Compound II Phosphate Salt Hemihydrate Form A according to Embodiment 159, characterized by an X-ray powder diffractogram comprising (a) a signal at the following two-theta values: 9.1±0.2, 16.7±0.2, and 18.7±0.2; and (b) a signal at one or more two-theta values chosen from 14.9±0.2, 15.7±0.2, and 20.0±0.2.
164. Compound II Phosphate Salt Hemihydrate Form A according to Embodiment 159, characterized by an X-ray powder diffractogram comprising signals at 9.1±0.2, 14.9±0.2, 15.7±0.2, 16.7±0.2, 18.7±0.2, and 20.0±0.2 two-theta.
165. Compound II Phosphate Salt Hemihydrate Form A according to Embodiment 159, characterized by an X-ray powder diffractogram comprising (a) a signal at the following two-theta values: 9.1±0.2, 16.7±0.2, and 18.7±0.2; and (b) a signal at one or more two-theta values chosen from 10.1±0.2, 14.9±0.2, 15.7±0.2, 18.4±0.2, and 20.0±0.2.
166. Compound II Phosphate Salt Hemihydrate Form A according to Embodiment 159, characterized by an X-ray powder diffractogram comprising signals at 9.1±0.2, 10.1±0.2, 14.9±0.2, 15.7±0.2, 16.7±0.2, 18.4±0.2, 18.7±0.2, and 20.0±0.2 two-theta.
167. Compound II Phosphate Salt Hemihydrate Form A according to Embodiment 159, characterized by an X-ray powder diffractogram substantially similar to that in FIG. 24.
168. Compound II Phosphate Salt Hemihydrate Form A according to any one of Embodiments 159-167, characterized by a ¹³C NMR spectrum comprising one or more signals chosen from 15.3±0.2 ppm, 15.8±0.2 ppm, 16.6±0.2 ppm, 39.9±0.2 ppm, and 141.3±0.2 ppm.
169. Compound II Phosphate Salt Hemihydrate Form A according to any one of Embodiments 159-167, characterized by a ¹³C NMR spectrum comprising one or more signals chosen from 15.3±0.2 ppm, 15.8±0.2 ppm, 16.6±0.2 ppm, 18.4±0.2 ppm, 38.6±0.2 ppm, 39.9±0.2 ppm, 126.6±0.2 ppm, 127.1±0.2 ppm, 136.8±0.2 ppm, and 141.3±0.2 ppm.
170. Compound II Phosphate Salt Hemihydrate Form A according to any one of Embodiments 159-167, characterized by a ¹³C NMR spectrum comprising signals at 15.3±0.2 ppm, 15.8±0.2 ppm, 16.6±0.2 ppm, 39.9±0.2 ppm, and 141.3±0.2 ppm.
171. Compound II Phosphate Salt Hemihydrate Form A according to any one of Embodiments 159-167, characterized by a ¹³C NMR spectrum comprising signals at 15.3±0.2 ppm, 15.8±0.2 ppm, 16.6±0.2 ppm, 18.4±0.2 ppm, 38.6±0.2 ppm, 39.9±0.2 ppm, 126.6±0.2 ppm, 127.1±0.2 ppm, 136.8±0.2 ppm, and 141.3±0.2 ppm.
172. Compound II Phosphate Salt Hemihydrate Form A according to any one of Embodiments 159-167, characterized by a ¹³C NMR spectrum substantially similar to that in FIG. 25.
173. Compound II Phosphate Salt Hemihydrate Form A according to any one of Embodiments 159-172, characterized by a ¹³C NMR spectrum measured after dehydration comprising one or more signals chosen from 16.5±0.2 ppm, 38.5±0.2 ppm, 39.3±0.2 ppm, 125.6±ppm, and 127.5±0.2 ppm.
174. Compound II Phosphate Salt Hemihydrate Form A according to any one of Embodiments 159-172, characterized by a ¹³C NMR spectrum measured after dehydration comprising one or more signals chosen from 16.5±0.2 ppm, 36.6±0.2 ppm, 37±0.2 ppm, 38.5±0.2 ppm, 39.3±0.2 ppm, 125.6±0.2 ppm, 127.5±0.2 ppm, 136.8±0.2 ppm, 141.3±0.2 ppm, and 143±0.2 ppm.
175. Compound II Phosphate Salt Hemihydrate Form A according to any one of Embodiments 159-172, characterized by a ¹³C NMR spectrum measured after dehydration comprising signals at 16.5±0.2 ppm, 38.5±0.2 ppm, 39.3±0.2 ppm, 125.6±ppm, and 127.5±0.2 ppm.
176. Compound II Phosphate Salt Hemihydrate Form A according to any one of Embodiments 159-172, characterized by a ¹³C NMR spectrum measured after dehydration comprising signals at 16.5±0.2 ppm, 36.6±0.2 ppm, 37±0.2 ppm, 38.5±0.2 ppm, 39.3±0.2 ppm, 125.6±0.2 ppm, 127.5±0.2 ppm, 136.8±0.2 ppm, 141.3±0.2 ppm, and 143±0.2 ppm.
177. Compound II Phosphate Salt Hemihydrate Form A according to any one of Embodiments 159-172, characterized by a ¹³C NMR spectrum measured after dehydration substantially similar to that in FIG. 26.
178. Compound II Phosphate Salt Hemihydrate Form A according to any one of Embodiments 159-177, characterized by a ³¹P NMR spectrum comprising one or more signals chosen from −1.8±0.2 ppm, −1.1±0.2 ppm, and 3.1±0.2 ppm.
179. Compound II Phosphate Salt Hemihydrate Form A according to any one of Embodiments 159-177, characterized by a ³¹P NMR spectrum comprising signals at −1.8±0.2 ppm, −1.1±0.2 ppm, and 3.1±0.2 ppm.
180. Compound II Phosphate Salt Hemihydrate Form A according to any one of Embodiments 159-177, characterized by a ³¹P NMR spectrum substantially similar to that in FIG. 27A.
181. Compound II Phosphate Salt Hemihydrate Form A according to any one of Embodiments 159-180, characterized by a ³¹P NMR spectrum measured after dehydration comprising one or more signals chosen from 3.0±0.2 ppm, 3.2±0.2 ppm, 4.4±0.2 ppm, and 5.6±0.2 ppm.
182. Compound II Phosphate Salt Hemihydrate Form A according to any one of Embodiments 159-180, characterized by a ³¹P NMR spectrum measured after dehydration comprising signals at 3.0±0.2 ppm, 3.2±0.2 ppm, 4.4±0.2 ppm, and 5.6±0.2 ppm.
183. Compound II Phosphate Salt Hemihydrate Form A according to any one of Embodiments 159-180, characterized by a ³¹P NMR spectrum measured after dehydration substantially similar to that in FIG. 27B.
184. Compound II Phosphate Salt Hemihydrate Form A according to any one of Embodiments 159-183, characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 9.2 ± 0.1 Å | α | 90° |
| b | 23.5 ± 0.1 Å | β | 90° |
| c | 38.3 ± 0.1 Å | γ | 90°. |

185. A pharmaceutical composition comprising Compound II Phosphate Salt Hemihydrate Form A according to any one of Embodiments 159-184 and a pharmaceutically acceptable carrier.
186. A method of treating an APOL1 mediated disease comprising administering to a patient in need thereof Compound II Phosphate Salt Hemihydrate Form A according to any one of Embodiments 159-184 or a pharmaceutical composition according to Embodiment 185.
187. The method according to Embodiment 186, wherein the APOL1 mediated disease is an APOL1 mediated kidney disease.
188. The method according to Embodiment 187, wherein the APOL1 mediated kidney disease is chosen from ESKD, NDKD, FSGS, HIV-associated nephropathy, arterionephrosclerosis, lupus nephritis, microalbuminuria, and chronic kidney disease.
189. The method according to Embodiment 187 or 188, wherein the APOL1 mediated kidney disease is FSGS or NDKD.
190. The method according to any one of Embodiments 187-189, wherein the APOL1 mediated kidney disease is associated with APOL1 genetic alleles chosen from homozygous G1: S342G:I384M and homozygous G2: N388del:Y389del.
191. The method according to any one of Embodiments 187-189, wherein the APOL1 mediated kidney disease is associated with compound heterozygous G1: S342G:I384M and G2: N388del:Y389del APOL1 genetic alleles.
192. The method according to Embodiment 186, wherein the APOL1 mediated disease is cancer.
193. The method according to Embodiment 186 or 192, wherein the APOL1 mediated disease is pancreatic cancer.
194. A method of inhibiting APOL1 activity comprising contacting said APOL1 with Compound II Phosphate Salt Hemihydrate Form A according to any one of Embodiments 159-184 or a pharmaceutical composition according to Embodiment 185.
195. The method according to Embodiment 194, wherein the APOL1 is associated with APOL1 genetic alleles chosen from homozygous G1: S342G:I384M and homozygous G2: N388del:Y389del.
196. The method according to Embodiment 194, wherein the APOL1 is associated with compound heterozygous G1: S342G:I384M and G2: N388del:Y389del APOL1 genetic alleles.
197. Use of Compound II Phosphate Salt Hemihydrate Form A according to any one of Embodiments 159-184 in the manufacture of a medicament for treating APOL1 mediated disease (e.g., APOL1 mediated kidney disease).
198. Compound II Phosphate Salt Hemihydrate Form A according to any one of Embodiments 159-184 for use in treating APOL1 mediated disease (e.g., APOL1 mediated kidney disease).
199. A method of preparing Compound II Phosphate Salt Hemihydrate Form A comprising:

adding Compound II free form Hemihydrate Form A to 2-MeTHF to form a solution;

adding H₃PO₄ dropwise to the solution;

stirring the solution at ambient temperature;

collecting a solid material by centrifugation; and

drying the solid material.

200. A method of preparing Compound II Phosphate Salt Hemihydrate Form A comprising:

charging Compound II free form Hemihydrate Form A and 2-MeTHF to a reactor;

agitating the reactor at about 40° C.;

seeding the reactor with Compound II Phosphate Salt Hemihydrate Form A;

slowly adding a phosphoric acid solution to the reactor to form a slurry;

cooling the slurry;

agitating the cooled slurry and filtering under vacuum to yield a wet cake; and

drying the wet cake.

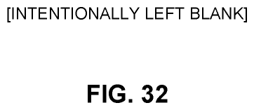

201. Compound II free form Hemihydrate Form A.
202. Compound II free form Hemihydrate Form A according to Embodiment 201, characterized by an X-ray powder diffractogram measured at ambient temperature comprising a signal at 17.1±0.2, 19.1±0.2, and/or 20.4±0.2 two-theta.
203. Compound II free form Hemihydrate Form A according to Embodiment 201, characterized by an X-ray powder diffractogram measured at ambient temperature comprising at two or more two-theta values chosen from 17.1±0.2, 19.1±0.2, and 20.4±0.2.
204. Compound II free form Hemihydrate Form A according to Embodiment 201, characterized by an X-ray powder diffractogram measured at ambient temperature comprising signals at 17.1±0.2, 19.1±0.2, and 20.4±0.2 two-theta.
205. Compound II free form Hemihydrate Form A according to Embodiment 201, characterized by an X-ray powder diffractogram measured at ambient temperature comprising (a) a signal at the following two-theta values: 17.1±0.2, 19.1±0.2, and 20.4±0.2; and (b) a signal at one or more two-theta values chosen from 5.7±0.2, 6.5±0.2, and 14.4±0.2.
206. Compound II free form Hemihydrate Form A according to Embodiment 201, characterized by an X-ray powder diffractogram measured at ambient temperature comprising signals at 5.7±0.2, 6.5±0.2, 14.4±0.2, 17.1±0.2, 19.1±0.2, and 20.4±0.2 two-theta.
207. Compound II free form Hemihydrate Form A according to Embodiment 201, characterized by an X-ray powder diffractogram measured at ambient temperature comprising (a) a signal at the following two-theta values: 17.1±0.2, 19.1±0.2, and 20.4±0.2; and (b) a signal at one or more two-theta values chosen from 5.7±0.2, 6.5±0.2, 11.4±0.2, 12.1±0.2, and 14.4±0.2.
208. Compound II free form Hemihydrate Form A according to Embodiment 201, characterized by an X-ray powder diffractogram measured at ambient temperature comprising signals at 5.7±0.2, 6.5±0.2, 11.4±0.2, 12.1±0.2, 14.4±0.2, 17.1±0.2, 19.1±0.2, and 20.4±0.2 two-theta.
209. Compound II free form Hemihydrate Form A according to Embodiment 201, characterized by an X-ray powder diffractogram measured at ambient temperature substantially similar to that in FIG. 30A.
210. Compound II free form Hemihydrate Form A according to any one of Embodiments 201-209, characterized by an X-ray powder diffractogram measured at a temperature in the range of 40° C. to 50° C. comprising a signal at 11.3±0.2, 19.0±0.2, and/or 20.1±0.2 two-theta.
211. Compound II free form Hemihydrate Form A according to any one of Embodiments 201-209, characterized by an X-ray powder diffractogram measured at a temperature in the range of 40° C. to 50° C. comprising at two or more two-theta values chosen from 11.3±0.2, 19.0±0.2, and 20.1±0.2.
212. Compound II free form Hemihydrate Form A according to any one of Embodiments 201-209, characterized by an X-ray powder diffractogram measured at a temperature in the range of 40° C. to 50° C. comprising signals at 11.3±0.2, 19.0±0.2, and 20.1±0.2 two-theta.
213. Compound II free form Hemihydrate Form A according to any one of Embodiments 201-209, characterized by an X-ray powder diffractogram measured at a temperature in the range of 40° C. to 50° C. comprising (a) a signal at the following two-theta values: 11.3±0.2, 19.0±0.2, and 20.1±0.2; and (b) a signal at one or more two-theta values chosen from 5.6±0.2, 22.3±0.2, and 25.1±0.2.
214. Compound II free form Hemihydrate Form A according to any one of Embodiments 201-209, characterized by an X-ray powder diffractogram measured at a temperature in the range of 40° C. to 50° C. comprising signals at 5.6±0.2, 11.3±0.2, 19.0±0.2, 20.1±0.2, 22.3±0.2, and 25.1±0.2 two-theta.
215. Compound II free form Hemihydrate Form A according to any one of Embodiments 201-209, characterized by an X-ray powder diffractogram measured at a temperature in the range of 40° C. to 50° C. comprising (a) a signal at the following two-theta values: 11.3±0.2, 19.0±0.2, and 20.1±0.2; and (b) a signal at one or more two-theta values chosen from 5.6±0.2, 22.3±0.2, 24.8±0.2, 25.1±0.2, and 27.8±0.2.
216. Compound II free form Hemihydrate Form A according to any one of Embodiments 201-209, characterized by an X-ray powder diffractogram measured at a temperature in the range of 40° C. to 50° C. comprising signals at 5.6±0.2, 11.3±0.2, 19.0±0.2, 20.1±0.2, 22.3±0.2, 24.8±0.2, 25.1±0.2, and 27.8±0.2 two-theta.
217. Compound II free form Hemihydrate Form A according to any one of Embodiments 201-209, characterized by an X-ray powder diffractogram measured at a temperature in the range of 40° C. to 50° C. substantially similar to that in FIG. 30B.
218. Compound II free form Hemihydrate Form A according to any one of Embodiments 201-217, characterized by an X-ray powder diffractogram measured at a temperature in the range of 60° C. to 90° C. comprising a signal at 5.5±0.2, 19.2±0.2, and/or 19.8±0.2 two-theta.
219. Compound II free form Hemihydrate Form A according to any one of Embodiments 201-217, characterized by an X-ray powder diffractogram measured at a temperature in the range of 60° C. to 90° C. comprising at two or more two-theta values chosen from 5.5±0.2, 19.2±0.2, and 19.8±0.2.
220. Compound II free form Hemihydrate Form A according to any one of Embodiments 201-217, characterized by an X-ray powder diffractogram measured at a temperature in the range of 60° C. to 90° C. comprising signals at 5.5±0.2, 19.2±0.2, and 19.8±0.2 two-theta.
221. Compound II free form Hemihydrate Form A according to any one of Embodiments 201-217, characterized by an X-ray powder diffractogram measured at a temperature in the range of 60° C. to 90° C. comprising (a) a signal at the following two-theta values: 5.5±0.2, 19.2±0.2, and 19.8±0.2; and (b) a signal at one or more two-theta values chosen from 11.0±0.2, 21.8±0.2, and 27.2±0.2.
222. Compound II free form Hemihydrate Form A according to any one of Embodiments 201-217, characterized by an X-ray powder diffractogram measured at a temperature in the range of 60° C. to 90° C. comprising signals at 5.5±0.2, 11.0±0.2, 19.2±0.2, 19.8±0.2, 21.8±0.2, and 27.2±0.2 two-theta.
223. Compound II free form Hemihydrate Form A according to any one of Embodiments 201-217, characterized by an X-ray powder diffractogram measured at a temperature in the range of 60° C. to 90° C. comprising (a) a signal at the following two-theta values: 5.5±0.2, 19.2±0.2, and 19.8±0.2; and (b) a signal at one or more two-theta values chosen from 11.0±0.2, 19.0±0.2, 21.8±0.2, 24.7±0.2, and 27.2±0.2.
224. Compound II free form Hemihydrate Form A according to any one of Embodiments 201-217, characterized by an X-ray powder diffractogram measured at a temperature in the range of 60° C. to 90° C. comprising signals at 5.5±0.2, 11.0±0.2, 19.0±0.2, 19.2±0.2, 19.8±0.2, 21.8±0.2, 24.7±0.2, and 27.2±0.2 two-theta.
225. Compound II free form Hemihydrate Form A according to any one of Embodiments 201-217, characterized by an X-ray powder diffractogram measured at a temperature in the range of 60° C. to 90° C. substantially similar to that in FIG. 30C.
226. Compound II free form Hemihydrate Form A according to any one of Embodiments 201-225, characterized by a ¹³C NMR spectrum comprising one or more signals chosen from 21.9±0.2 ppm, 22.6±0.2 ppm, 133.2±0.2 ppm, 139.8±0.2 ppm, and 140.9±0.2 ppm.
227. Compound II free form Hemihydrate Form A according to any one of Embodiments 201-225, characterized by a ¹³C NMR spectrum comprising one or more signals chosen from 21.9±0.2 ppm, 22.6±0.2 ppm, 38.4±0.2 ppm, 124.2±0.2 ppm, 124.7±0.2 ppm, 133.2±0.2 ppm, 139.8±0.2 ppm, 140.9±0.2 ppm, 142.7±0.2 ppm, and 147.6±0.2 ppm.
228. Compound II free form Hemihydrate Form A according to any one of Embodiments 201-225, characterized by a ¹³C NMR spectrum comprising signals at 21.9±0.2 ppm, 22.6±0.2 ppm, 133.2±0.2 ppm, 139.8±0.2 ppm, and 140.9±0.2 ppm.
229. Compound II free form Hemihydrate Form A according to any one of Embodiments 201-225, characterized by a ¹³C NMR spectrum comprising signals at 21.9±0.2 ppm, 22.6±0.2 ppm, 38.4±0.2 ppm, 124.2±0.2 ppm, 124.7±0.2 ppm, 133.2±0.2 ppm, 139.8±0.2 ppm, 140.9±0.2 ppm, 142.7±0.2 ppm, and 147.6±0.2 ppm.
230. Compound II free form Hemihydrate Form A according to any one of Embodiments 201-225, characterized by a ¹³C NMR spectrum substantially similar to that in FIG. 31.
231. Compound II free form Hemihydrate Form A according to any one of Embodiments 201-230, characterized by a ¹³C NMR spectrum measured after dehydration (weekend at ambient temperature and overnight at about 80° C. in rotor) comprising one or more signals chosen from 22.4±0.2 ppm, 22.6±0.2 ppm, 38.5±0.2 ppm, 132.9±0.2 ppm, and 139.4±0.2 ppm.
232. Compound II free form Hemihydrate Form A according to any one of Embodiments 201-230, characterized by a ¹³C NMR spectrum measured after dehydration (weekend at ambient temperature and overnight at about 80° C. in rotor) comprising one or more signals chosen from 22.4±0.2 ppm, 22.6±0.2 ppm, 35.3±0.2 ppm, 38.5±0.2 ppm, 39.8±0.2 ppm, 124.4±0.2 ppm, 132.9±0.2 ppm, 139.4±0.2 ppm, 141.5±0.2 ppm, and 142.2±0.2 ppm.
233. Compound II free form Hemihydrate Form A according to any one of Embodiments 201-230, characterized by a ¹³C NMR spectrum measured after dehydration (weekend at ambient temperature and overnight at about 80° C. in rotor) comprising signals at 22.4±0.2 ppm, 22.6±0.2 ppm, 38.5±0.2 ppm, 132.9±0.2 ppm, and 139.4±0.2 ppm.
234. Compound II free form Hemihydrate Form A according to any one of Embodiments 201-230, characterized by a ¹³C NMR spectrum measured after dehydration (weekend at ambient temperature and overnight at about 80° C. in rotor) comprising signals at 22.4±0.2 ppm, 22.6±0.2 ppm, 35.3±0.2 ppm, 38.5±0.2 ppm, 39.8±0.2 ppm, 124.4±0.2 ppm, 132.9±0.2 ppm, 139.4±0.2 ppm, 141.5±0.2 ppm, and 142.2±0.2 ppm.
235. Compound II free form Hemihydrate Form A according to any one of Embodiments 201-230, characterized by a ¹³C NMR spectrum measured after dehydration (weekend at ambient temperature and overnight at about 80° C. in rotor) substantially similar to that in FIG. 32.
236. Compound II free form Hemihydrate Form A according to any one of Embodiments 201-235, characterized by a monoclinic crystal system, a P21 space group, and unit cell dimensions measured at 100 K on a Bruker diffractometer equipped Cu Kα radiation (λ=1.54178 Å) of:

| | | | |
|---|---|---|---|
| a | 13.8 ± 0.1 Å | α | 90° |
| b | 8.1 ± 0.1 Å | β | 100.2 ± 0.1° |
| c | 15.6 ± 0.1 Å | γ | 90°. |

237. A pharmaceutical composition comprising Compound II free form Hemihydrate Form A according to any one of Embodiments 201-236 and a pharmaceutically acceptable carrier.
238. A method of treating an APOL1 mediated disease comprising administering to a patient in need thereof Compound II free form Hemihydrate Form A according to any one of Embodiments 201-236 or a pharmaceutical composition according to Embodiment 237.
239. The method according to Embodiment 238, wherein the APOL1 mediated disease is an APOL1 mediated kidney disease.
240. The method according to Embodiment 239, wherein the APOL1 mediated kidney disease is chosen from ESKD, NDKD, FSGS, HIV-associated nephropathy, arterionephrosclerosis, lupus nephritis, microalbuminuria, and chronic kidney disease.
241. The method according to Embodiment 239 or 240, wherein the APOL1 mediated kidney disease is FSGS or NDKD.
242. The method according to any one of Embodiments 239-241, wherein the APOL1 mediated kidney disease is associated with APOL1 genetic alleles chosen from homozygous G1: S342G:1384M and homozygous G2: N388del:Y389del.
243. The method according to any one of Embodiments 239-241, wherein the APOL1 mediated kidney disease is associated with compound heterozygous G1: S342G:1384M and G2: N388del:Y389del APOL1 genetic alleles.
244. The method according to Embodiment 238, wherein the APOL1 mediated disease is cancer.
245. The method according to Embodiment 238 or 244, wherein the APOL1 mediated disease is pancreatic cancer.
246. A method of inhibiting APOL1 activity comprising contacting said APOL1 with Compound II free form Hemihydrate Form A according to any one of Embodiments 201-236 or a pharmaceutical composition according to Embodiment 237.
247. The method according to Embodiment 246, wherein the APOL1 is associated with APOL1 genetic alleles chosen from homozygous G1: S342G:1384M and homozygous G2: N388del:Y389del.
248. The method according to Embodiment 246, wherein the APOL1 is associated with compound heterozygous G1: S342G:1384M and G2: N388del:Y389del APOL1 genetic alleles.
249. Use of Compound II free form Hemihydrate Form A according to any one of Embodiments 201-236 in the manufacture of a medicament for treating APOL1 mediated disease (e.g., APOL1 mediated kidney disease).
250. Compound II free form Hemihydrate Form A according to any one of Embodiments 201-236 for use in treating APOL1 mediated disease (e.g., APOL1 mediated kidney disease).
251. A method of preparing Compound II free form Hemihydrate Form A comprising:

adding Amorphous free form Compound II to MEK to produce a solution;

adding water and n-Heptane to the solution;

stirring the solution at ambient temperature;

filtering the solution to obtain a solid material; and

drying the solid material.

252. Compound II free form Form C.
253. Compound II free form Form C according to Embodiment 252, characterized by an X-ray powder diffractogram measured at ambient temperature comprising a signal at 13.0±0.2 two-theta.
254. Compound II free form Form C according to Embodiment 252, characterized by an X-ray powder diffractogram measured at ambient temperature comprising a signal at 18.5±0.2 two-theta.
255. Compound II free form Form C according to Embodiment 252, characterized by an X-ray powder diffractogram comprising a signal at 21.6±0.2 two-theta.
256. Compound II free form Form C according to Embodiments 252, characterized by an X-ray powder diffractogram comprising a signal at each of two-theta values 13.0±0.2, 18.5±0.2, and 21.6±0.2.
257. Compound II free form Form C according to Embodiment 252, characterized by an X-ray powder diffractogram comprising a signal at one or more two-theta values chosen from 13.0±0.2, 17.7±0.2, 18.5±0.2, and 21.6±0.2.
258. Compound II free form Form C according to Embodiment 252, characterized by an X-ray powder diffractogram comprising signals at two or more two-theta values chosen from 13.0±0.2, 17.7±0.2, 18.5±0.2, and 21.6±0.2.
259. Compound II free form Form C according to Embodiment 252, characterized by an X-ray powder diffractogram comprising signals at three or more two-theta values chosen from 13.0±0.2, 17.7±0.2, 18.5±0.2, and 21.6±0.2.
260. Compound II free form Form C according to Embodiment 252, characterized by an X-ray powder diffractogram comprising signals at two-theta values 13.0±0.2, 17.7±0.2, 18.5±0.2, and 21.6±0.2.
261. Compound II free form Form C according to Embodiment 252, characterized by an X-ray powder diffractogram comprising a signal at one or more two-theta values chosen from 13.0±0.2, 15.7±0.2, 17.7±0.2, 18.5±0.2, 19.8±0.2, 21.6±0.2 and 23.6±0.2.
262. Compound II free form Form C according to Embodiment 252, characterized by an X-ray powder diffractogram comprising signals at two or more two-theta values chosen from 13.0±0.2, 15.7±0.2, 17.7±0.2, 18.5±0.2, 19.8±0.2, 21.6±0.2 and 23.6±0.2.
263. Compound II free form Form C according to Embodiment 252, characterized by an X-ray powder diffractogram comprising signals at three or more two-theta values chosen from 13.0±0.2, 15.7±0.2, 17.7±0.2, 18.5±0.2, 19.8±0.2, 21.6±0.2 and 23.6±0.2.
264. Compound II free form Form C according to Embodiment 252, characterized by an X-ray powder diffractogram comprising signals at four or more two-theta values chosen from 13.0±0.2, 15.7±0.2, 17.7±0.2, 18.5±0.2, 19.8±0.2, 21.6±0.2 and 23.6±0.2.
265. Compound II free form Form C according to Embodiment 252, characterized by an X-ray powder diffractogram comprising signals at five or more two-theta values chosen from 13.0±0.2, 15.7±0.2, 17.7±0.2, 18.5±0.2, 19.8±0.2, 21.6±0.2 and 23.6±0.2.
266. Compound II free form Form C according to Embodiment 252, characterized by an X-ray powder diffractogram comprising signals at six or more two-theta values chosen from 13.0±0.2, 15.7±0.2, 17.7±0.2, 18.5±0.2, 19.8±0.2, 21.6±0.2 and 23.6±0.2.
267. Compound II free form Form C according to Embodiment 252, characterized by an X-ray powder diffractogram comprising signals at at two-theta values 13.0±0.2, 15.7±0.2, 17.7±0.2, 18.5±0.2, 19.8±0.2, 21.6±0.2 and 23.6±0.2.
268. Compound II free form Form C according to Embodiment 252, characterized by an X-ray powder diffractogram comprising (a) a signal at one, two or three of the following two-theta values: 13.0±0.2, 18.5±0.2, and 21.6±0.2; and (b) a signal at one or more two-theta values chosen from 11.1±0.2, 15.5±0.2, and 15.7±0.2, 16.5±0.2, 17.1±0.2, 17.7±0.2, 17.9±0.2, 19.8±0.2, 22.0±0.2, 23.3±0.2, 23.6±0.2, 24.0±0.2, 26.3±0.2, 26.7±0.2, 26.8±0.2, 30.6±0.2.
269. Compound II free form Form C according to Embodiment 252, characterized by an X-ray powder diffractogram comprising (a) a signal at each of two-theta values 13.0±0.2, 18.5±0.2, and 21.6±0.2; and (b) a signal at one or more two-theta values chosen from 11.1±0.2, 15.5±0.2, and 15.7±0.2, 16.5±0.2, 17.1±0.2, 17.7±0.2, 17.9±0.2, 19.8±0.2, 22.0±0.2, 23.3±0.2, 23.6±0.2, 24.0±0.2, 26.3±0.2, 26.7±0.2, 26.8±0.2, 30.6±0.2.
270. Compound II free form Form C according to Embodiment 252, characterized by an X-ray powder diffractogram substantially similar to that in FIG. 35.
271. Compound II free form Form C according to any one of Embodiments 252 to 270, characterized by a TGA thermogram showing negligible weight loss from ambient temperature up to 200° C.
272. Compound II free form Form C according to any one of Embodiments 252 to 271, characterized by a TGA thermogram substantially similar to that in FIG. 36.
273. Compound II free form Form C according to any one of Embodiments 252 to 272, characterized by a DSC curve having an endothermic peak at about 218° C.
274. Compound II free form Form C according to any one of Embodiments 252 to 273, characterized by a DSC curve substantially similar to that in FIG. 37.
275. Compound II free form Form C according to any one of Embodiments 252 to 274, characterized by a ¹³C NMR spectrum comprising one or more signals chosen from 149.3±0.2 ppm, 144.3±0.2 ppm, 135.0±0.2 ppm, 127.2±0.2 ppm, and 124.5±0.2 ppm.
276. Compound II free form Form C according to any one of Embodiments 252 to 274, characterized by a ¹³C NMR spectrum comprising signals at 149.3±0.2 ppm, 144.3±0.2 ppm, 135.0±0.2 ppm, 127.2±0.2 ppm, and 124.5±0.2 ppm.
277. Compound II free form Form C according to any one of Embodiments 252 to 274, characterized by a ¹³C NMR spectrum comprising one or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
278. Compound II free form Form C according to any one of Embodiments 252 to 274, characterized by a ¹³C NMR spectrum comprising two or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
279. Compound II free form Form C according to any one of Embodiments 252 to 274, characterized by a ¹³C NMR spectrum comprising three or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
280. Compound II free form Form C according to any one of Embodiments 252 to 274, characterized by a ¹³C NMR spectrum comprising four or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
281. Compound II free form Form C according to any one of Embodiments 252 to 274, characterized by a ¹³C NMR spectrum comprising five or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
282. Compound II free form Form C according to any one of Embodiments 252 to 274, characterized by a ¹³C NMR spectrum comprising six or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
283. Compound II free form Form C according to any one of Embodiments 252 to 274, characterized by a ¹³C NMR spectrum comprising seven or more signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
284. Compound II free form Form C according to any one of Embodiments 252 to 274, characterized by a ¹³C NMR spectrum comprising signals at 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
285. Compound II free form Form C according to any one of Embodiments 252 to 274, characterized by a ¹³C NMR spectrum comprising (a) one or more (e.g., two or more, three or more, four or more, etc.) signals chosen from 149.3±0.2 ppm, 144.3±0.2 ppm, 135.0±0.2 ppm, 127.2±0.2 ppm, and 124.5±0.2 ppm; and (b) one or more (e.g., two or more, three or more, four or more, etc.) signals chosen from 74.0±0.2 ppm, 66.9±0.2 ppm, 62.0±0.2 ppm, 49.4±0.2 ppm, 47.8±0.2 ppm, 37.7±0.2 ppm, 36.8±0.2 ppm, and 25.9±0.2 ppm.
286. Compound II free form Form C according to any one of Embodiments 252 to 285, characterized by a ¹³C NMR spectrum substantially similar to that in FIG. 38.
287. Compound II free form Form C according to any one of Embodiments 252 to 286, having a single crystal unit cell characterized by an orthorhombic crystal system, a P2₁2₁2₁ space group, and unit cell dimensions measured at 298 K on a Bruker diffractometer equipped Cu Kα radiation (λ=1.54178 Å) of:

| | | | |
|---|---|---|---|
| a | 10.3 ± 0.1 Å | α | 90° |
| b | 12.5 ± 0.1 Å | β | 90° |
| c | 12.8 ± 0.1 Å | γ | 90°. |

288. Compound II free form Form C according to any one of Embodiments 252 to 286, having a single crystal unit cell characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 10.3 ± 0.1 Å | α | 90° |
| b | 12.3 ± 0.1 Å | β | 90° |
| c | 12.7 ± 0.1 Å | γ | 90°. |

289. A method of preparing Compound II free form Form C comprising:

adding 0.5 ml MEK to Compound II free form Hemihydrate Form A;

stirring at 20° C. overnight; and

isolating Compound II free form Form C.

290. A pharmaceutical composition comprising Compound II free form Form C according to any one of Embodiments 252-288 and a pharmaceutically acceptable carrier.
291. A method of treating an APOL1 mediated disease comprising administering to a patient in need thereof Compound II free form Form C according to any one of Embodiments 252-288 or a pharmaceutical composition according to Embodiment 290.
292. The method according to Embodiment 291, wherein the APOL1 mediated disease is an APOL1 mediated kidney disease.
293. The method according to Embodiment 292, wherein the APOL1 mediated kidney disease is chosen from ESKD, NDKD, FSGS, HIV-associated nephropathy, arterionephrosclerosis, lupus nephritis, microalbuminuria, and chronic kidney disease.
294. The method according to Embodiment 291 or 292, wherein the APOL1 mediated kidney disease is FSGS or NDKD.
295. The method according to any one of Embodiments 291-294, wherein the APOL1 mediated kidney disease is associated with APOL1 genetic alleles chosen from homozygous G1: S342G:I384M and homozygous G2: N388del:Y389del.
296. The method according to any one of Embodiments 291-294, wherein the APOL1 mediated kidney disease is associated with compound heterozygous G1: S342G:I384M and G2: N388del:Y389del APOL1 genetic alleles.
297. The method according to Embodiment 291, wherein the APOL1 mediated disease is cancer.
298. The method according to Embodiment 291 or 297, wherein the APOL1 mediated disease is pancreatic cancer.
299. A method of inhibiting APOL1 activity comprising contacting said APOL1 with Compound II free form Hemihydrate Form A according to any one of Embodiments 252-288 or a pharmaceutical composition according to Embodiment 290.
300. The method according to Embodiment 299, wherein the APOL1 is associated with APOL1 genetic alleles chosen from homozygous G1: S342G:1384M and homozygous G2: N388del:Y389del.
301. The method according to Embodiment 299, wherein the APOL1 is associated with compound heterozygous G1: S342G:1384M and G2: N388del:Y389del APOL1 genetic alleles.
302. Use of Compound II free form Form C according to any one of Embodiments 252-288 in the manufacture of a medicament for treating APOL1 mediated disease (e.g., APOL1 mediated kidney disease).
303. Compound II free form Form C according to any one of Embodiments 252-288 for use in treating APOL1 mediated disease (e.g., APOL1 mediated kidney disease).
304. Compound II free form Form A.
305. Compound II free form Form A according to Embodiment 304, characterized by an X-ray powder diffractogram measured at ambient temperature comprising a signal at 9.1±0.2 two-theta.
306. Compound II free form Form A according to Embodiment 304, characterized by an X-ray powder diffractogram measured at ambient temperature comprising a signal at 11.7±0.2 two-theta.
307. Compound II free form Form A according to Embodiment 304, characterized by an X-ray powder diffractogram comprising a signal at 13.9±0.2 two-theta.
308. Compound II free form Form A according to Embodiment 304, characterized by an X-ray powder diffractogram comprising a signal at 14.1±0.2 two-theta.
309. Compound II free form Form A according to Embodiments 304, characterized by an X-ray powder diffractogram comprising a signal at two or more of two-theta values 9.1±0.2, 11.7±0.2, 13.9±0.2, and 14.1±0.2.
310. Compound II free form Form A according to Embodiment 304, characterized by an X-ray powder diffractogram comprising a signal at three or more two-theta values chosen from 9.1±0.2, 11.7±0.2, 13.9±0.2, and 14.1±0.2.
311. Compound II free form Form A according to Embodiment 304, characterized by an X-ray powder diffractogram comprising signals at 9.1±0.2 two-theta, 11.7±0.2 two-theta, 13.9±0.2 two-theta, and 14.1±0.2 two-theta.
312. Compound II free form Form A according to Embodiment 304, characterized by an X-ray powder diffractogram comprising (a) a signal at one or more two-theta values chosen from 9.1±0.2, 11.7±0.2, 13.9±0.2, 14.1±0.2, and 20.5±0.2; and (b) a signal at one or more (e.g., two, three, four, or five) two-theta values chosen from 16.6±0.2, 17.3±0.2, 18.3±0.2, 22.1±0.2, and 24.4±0.2.
313. Compound II free form Form A according to Embodiment 304, characterized by an X-ray powder diffractogram comprising (a) a signal at two or more two-theta values chosen from 9.1±0.2, 11.7±0.2, 13.9±0.2, 14.1±0.2, and 20.5±0.2; and (b) a signal at one or more (e.g., two, three, four, or five) two-theta values chosen from 16.6±0.2, 17.3±0.2, 18.3±0.2, 22.1±0.2, and 24.4±0.2.
314. Compound II free form Form A according to Embodiment 304, characterized by an X-ray powder diffractogram comprising (a) a signal at three or more two-theta values chosen from 9.1±0.2, 11.7±0.2, 13.9±0.2, 14.1±0.2, and 20.5±0.2; and (b) a signal at one or more (e.g., two, three, four, or five) two-theta values chosen from 16.6±0.2, 17.3±0.2, 18.3±0.2, 22.1±0.2, and 24.4±0.2.
315. Compound II free form Form A according to Embodiment 304, characterized by an X-ray powder diffractogram comprising (a) signals at 9.1±0.2 two-theta, 11.7±0.2 two-theta, 13.9±0.2 two-theta, 14.1±0.2 two-theta, and 20.5±0.2 two-theta; and (b) a signal at one or more (e.g., two, three, four, or five) two-theta values chosen from 16.6±0.2, 17.3±0.2, 18.3±0.2, 22.1±0.2, and 24.4±0.2.
316. Compound II free form Form A according to Embodiment 304, characterized by an X-ray powder diffractogram comprising signals at 9.1±0.2 two-theta, 11.7±0.2 two-theta, 13.9±0.2 two-theta, 14.1±0.2 two-theta, 16.6±0.2 two-theta, 17.3±0.2 two-theta, 18.3±0.2 two-theta, 22.1±0.2 two-theta, 20.5±0.2 two-theta, and 24.4±0.2 two-theta.
317. Compound II free form Form A according to Embodiment 304, characterized by an X-ray powder diffractogram substantially similar to that in FIG. 60.
318. Compound II free form Form A according to any one of Embodiments 304 to 317, characterized by a TGA thermogram showing negligible weight loss from ambient temperature up to 200° C.
319. Compound II free form Form A according to any one of Embodiments 304 to 317, characterized by a TGA thermogram substantially similar to that in FIG. 62.
320. Compound II free form Form A according to any one of Embodiments 304 to 319, characterized by a DSC curve having an endothermic peak at about 130° C.
321. Compound II free form Form A according to any one of Embodiments 304 to 319, characterized by a DSC curve substantially similar to that in FIG. 63.
322. Compound II free form Form A according to any one of Embodiments 304 to 321, characterized by a ¹³C NMR spectrum comprising one or more (e.g., two, three, four, five, six, seven, or eight) signals chosen from 143.6±0.2 ppm, 134.1±0.2 ppm, 128.8±0.2 ppm, 123.4±0.2 ppm, 68.3±0.2 ppm, 48.9±0.2 ppm, 39.1±0.2 ppm, and 21.6±0.2 ppm.
323. Compound II free form Form A according to any one of Embodiments 304 to 321, characterized by a ¹³C NMR spectrum comprising signals at 143.6±0.2 ppm, 134.1±0.2 ppm, 128.8±0.2 ppm, 123.4±0.2 ppm.
324. Compound II free form Form A according to any one of Embodiments 304 to 321, characterized by a ¹³C NMR spectrum comprising signals at 68.3±0.2 ppm, 48.9±0.2 ppm, 39.1±0.2 ppm, and 21.6±0.2 ppm.
325. Compound II free form Form A according to any one of Embodiments 304 to 321, characterized by a ¹³C NMR spectrum comprising signals at 143.6±0.2 ppm, 134.1±0.2 ppm, 128.8±0.2 ppm, 123.4±0.2 ppm, 68.3±0.2 ppm, 48.9±0.2 ppm, 39.1±0.2 ppm, and 21.6±0.2 ppm.
326. Compound II free form Form A according to any one of Embodiments 304 to 321, characterized by a ¹³C NMR spectrum substantially similar to that in FIG. 61.
327. Compound II free form Form A according to any one of Embodiments 304 to 325, having a single crystal unit cell characterized by a monoclinic crystal system, a/2 space group, and unit cell dimensions measured at 298 K on a Bruker diffractometer equipped Cu Kα radiation (λ=1.54178 Å) of:

| | | | |
|---|---|---|---|
| a | 10.1 ± 0.1 Å | α | 90° |
| b | 8.0 ± 0.1 Å | β | 101.0 ± 0.1° |
| c | 21.8 ± 0.1 Å | γ | 90°. |

328. A method of preparing Compound II free form Form A comprising:

desolvating Compound II free form MeOH Solvate in a 40° C. vacuum oven; and

isolating Compound II Form A.

329. Compound II free form Form B.
330. Compound II free form Form B according to Embodiment 329, characterized by a ¹³C NMR spectrum comprising one or more (e.g., two or more, three or more, four or more, five) signals chosen from 22.4±0.2 ppm, 22.6±0.2 ppm, 47.7±0.2 ppm, 64.1±0.2 ppm, and 74.6±0.2 ppm.
331. Compound II free form Form B according to Embodiment 329, characterized by a ¹³C NMR spectrum comprising signals at one or more (e.g., two or more, three or more, four or more, five) signals chosen from 22.4±0.2 ppm, 22.6±0.2 ppm, 38.5±0.2 ppm, 132.9±0.2 ppm, and 139.4±0.2 ppm.
332. Compound II free form Form B according to Embodiment 329, characterized by a ¹³C NMR spectrum comprising signals at one or more (e.g., two, three, four, five, six, seven, eight, nine, or ten) signals chosen from 22.4±0.2 ppm, 22.6±0.2 ppm, 44.3±0.2 ppm, 47.3±0.2 ppm, 47.7±0.2 ppm, 61.8±0.2 ppm, 64.1±0.2 ppm, 67.6±0.2 ppm, 74.6±0.2 ppm, and 139.4±0.2 ppm.
333. Compound II free form Form B according to Embodiment 329, characterized by a ¹³C NMR spectrum comprising signals at one or more (e.g., two, three, four, five, six, seven, eight, nine, or ten) signals chosen from 22.4±0.2 ppm, 22.6±0.2 ppm, 35.3±0.2 ppm, 38.5±0.2 ppm, 39.8±0.2 ppm, 124.4±0.2 ppm, 132.9±0.2 ppm, 139.4±0.2 ppm, 141.5±0.2 ppm, and 142.2±0.2 ppm.
334. Compound II free form Form B according to Embodiment 329, characterized by a ¹³C NMR spectrum comprising signals at 22.4±0.2 ppm, 22.6±0.2 ppm, 47.7±0.2 ppm, 64.1±0.2 ppm, and 74.6±0.2 ppm.
335. Compound II free form Form B according to Embodiment 329, characterized by a ¹³C NMR spectrum comprising signals at 22.4±0.2 ppm, 22.6±0.2 ppm, 38.5±0.2 ppm, 132.9±0.2 ppm, and 139.4±0.2 ppm.
336. Compound II free form Form B according to Embodiment 329, characterized by a ¹³C NMR spectrum comprising signals at 22.4±0.2 ppm, 22.6±0.2 ppm, 44.3±0.2 ppm, 47.3±0.2 ppm, 47.7±0.2 ppm, 61.8±0.2 ppm, 64.1±0.2 ppm, 67.6±0.2 ppm, 74.6±0.2 ppm, and 139.4±0.2 ppm.
337. Compound II free form Form B according to Embodiment 329, characterized by a ¹³C NMR spectrum comprising signals at 22.4±0.2 ppm, 22.6±0.2 ppm, 35.3±0.2 ppm, 38.5±0.2 ppm, 39.8±0.2 ppm, 124.4±0.2 ppm, 132.9±0.2 ppm, 139.4±0.2 ppm, 141.5±0.2 ppm, and 142.2±0.2 ppm.
338. Compound II free form Form B according to any one of Embodiments 329 to 338, characterized by a ¹³C NMR spectrum substantially similar to that in FIG. 64.
339. Compound II free form Form B according to any one of Embodiments 329 to 338, having a single crystal unit cell characterized by a monoclinic crystal system, a P21 space group, and unit cell dimensions measured at 100 K on a Bruker diffractometer equipped Cu Kα radiation (λ=1.54178 Å) of:

| | | | |
|---|---|---|---|
| a | 13.4 ± 0.1 Å | α | 90° |
| b | 8.1 ± 0.1 Å | β | 101.1 ± 0.1° |
| c | 16.0 ± 0.1 Å | γ | 90°. |

340. A method of preparing Compound II free form Form B comprising:

loading Compound II free form Hemihydrate Form A into an ssNMR rotor;

drying overnight in an 80° C. oven; and

sealing with rotor cap before removing solid from oven.

341. Compound II free form Quarter Hydrate.
342. Compound II free form Quarter Hydrate according to Embodiment 341, characterized by a ¹³C NMR spectrum comprising a signal at 64.5±0.2 ppm.
343. Compound II free form Quarter Hydrate according to Embodiment 341, characterized by a ¹³C NMR spectrum comprising one or more (e.g., two, three, or four) signals chosen from 151.8±0.2 ppm, 151.5±0.2 ppm, 121.1±0.2 ppm, and 35.3±0.2 ppm.
344. Compound II free form Quarter Hydrate according to Embodiment 341, characterized by a ¹³C NMR spectrum comprising signals at 151.8±0.2 ppm, 151.5±0.2 ppm, 121.1±0.2 ppm, 64.5±0.2 ppm, and 35.3±0.2 ppm.
345. Compound II free form Quarter Hydrate according to Embodiment 341, characterized by a ¹³C NMR spectrum comprising (a) one or more (e.g., two, three, or more, four) signals chosen from 151.8±0.2 ppm, 151.5±0.2 ppm, ppm, 121.1±0.2 ppm, and 35.3±0.2 ppm; and (b) one or more (e.g., two or more, three or more, four or more, five or more, six or more, seven or more, eight) signals at 74.4±0.2 ppm, 67.6±0.2 ppm, 64.5±0.2 ppm, 61.8±0.2 ppm, 47.5±0.2 ppm, 47.2±0.2 ppm, 44.1±0.2 ppm, and 22.1±0.2 ppm.
346. Compound II free form Quarter Hydrate according to Embodiment 341, characterized by a ¹³C NMR spectrum comprising (a) a signal at 64.5±0.2 ppm; and (b) one or more (e.g., two or more, three or more, four or more, five or more, six or more, seven) signals at 74.4±0.2 ppm, 67.6±0.2 ppm, 61.8±0.2 ppm, 47.5±0.2 ppm, 47.2±0.2 ppm, 44.1±0.2 ppm, and 22.1±0.2 ppm.
347. Compound II free form Quarter Hydrate according to any one of Embodiments 341 to 346, characterized by a ¹³C NMR spectrum substantially similar to that in FIG. 66.
348. Compound II free form Quarter Hydrate according to any one of Embodiments 341 to 347, having a single crystal unit cell characterized by a monoclinic crystal system, a P21 space group, and unit cell dimensions measured at 100 K on a Bruker diffractometer equipped Cu Kα radiation (λ=1.54178 Å) of:

| | | | |
|---|---|---|---|
| a | 18.9 ± 0.1 Å | α | 90° |
| b | 8.1 ± 0.1 Å | β | 99.1 ± 0.1° |
| c | 22.6 ± 0.1 Å | γ | 90°. |

349. A method of preparing Compound II free form Quarter Hydrate comprising:

dehydrating Compound II free form Hemihydrate Form A in isothermal 80° C. TGA;

unloading the solid to pack in the rotor as quickly as possible; and

sealing with rotor cap as soon as the solid was loaded.

350. Compound II free form Hydrate Mixture.
351. Compound II free form Hydrate Mixture according to Embodiment 350, characterized by an X-ray powder diffractogram measured at ambient temperature comprising a signal at 8.6±0.2 two-theta.
352. Compound II free form Hydrate Mixture according to Embodiment 350, characterized by an X-ray powder diffractogram measured at ambient temperature comprising a signal at 24.1±0.2 two-theta.
353. Compound II free form Hydrate Mixture according to Embodiment 350, characterized by an X-ray powder diffractogram comprising a signal at 24.5±0.2 two-theta.
354. Compound II free form Hydrate Mixture according to Embodiment 350, characterized by an X-ray powder diffractogram comprising a signal at 13.7±0.2 two-theta.
355. Compound II free form Hydrate Mixture according to Embodiment 350, characterized by an X-ray powder diffractogram comprising a signal at 3.6±0.2 two-theta.
356. Compound II free form Hydrate Mixture according to Embodiment 350, characterized by an X-ray powder diffractogram comprising a signal at 19.9±0.2 two-theta.
357. Compound II free form Hydrate Mixture according to Embodiments 350, characterized by an X-ray powder diffractogram comprising a signal at one or more (e.g., two or more, three or more, four or more, five or more, six) two-theta values chosen from 3.6±0.2, 8.6±0.2, 13.7±0.2, 19.9±0.2, 24.1±0.2, and 24.5±0.2.
358. Compound II free form Hydrate Mixture according to Embodiment 350, characterized by an X-ray powder diffractogram comprising signals at 3.6±0.2 two-theta, 8.6±0.2 two-theta, 13.7±0.2 two-theta, 19.9±0.2 two-theta, 24.1±0.2 two-theta, and 24.5±0.2 two-theta.
359. Compound II free form Hydrate Mixture according to Embodiment 350, characterized by an X-ray powder diffractogram comprising (a) a signal at one or more (e.g., two or more, three or more, four or more, five or more, six) two-theta values chosen from 3.6±0.2, 8.6±0.2, 13.7±0.2, 19.9±0.2, 24.1±0.2, and 24.5±0.2; and (b) a signal at one or more (e.g., two or more, three or more, four) two-theta values chosen from 22.2±0.2, 21.6±0.2, 17.0±0.2, and 14.6±0.2.
360. Compound II free form Hydrate Mixture according to Embodiment 350, characterized by an X-ray powder diffractogram comprising ((a) a signal at one or more (e.g., two or more, three or more, four or more, five or more, six) two-theta values chosen from 3.6±0.2, 8.6±0.2, 13.7±0.2, 19.9±0.2, 24.1±0.2, and 24.5±0.2; and (b) signals at 22.2±0.2 two-theta, 21.6±0.2 two-theta, 17.0±0.2 two-theta, and 14.6±0.2 two-theta.
361. Compound II free form Hydrate Mixture according to Embodiment 350, characterized by an X-ray powder diffractogram substantially similar to that in FIG. 67.
362. A method of preparing Compound II free form Hydrate Mixture comprising:

equilibrating Compound II Neat Form A for 3 days in a humidified chamber set at 95% RH; and

isolating the solid.

363. Compound II free form Monohydrate.
364. Compound II free form Monohydrate according to Embodiment 363, characterized by a ¹³C NMR spectrum comprising a signal at 134.1±0.2 ppm.
365. Compound II free form Monohydrate according to Embodiment 363, characterized by a ¹³C NMR spectrum comprising a signal at 21.1±0.2 ppm.
366. Compound II free form Monohydrate according to Embodiment 363, characterized by a ¹³C NMR spectrum comprising a signal at 134.1±0.2 ppm and a signal at 21.1±0.2 ppm.
367. Compound II free form Monohydrate according to Embodiment 363, characterized by a ¹³C NMR spectrum comprising (a) a signal at 134.1±0.2 ppm and/or a signal at 21.1±0.2 ppm; and (b) one or more signals (e.g., two, three, four, or five) signals chosen from 74.5±0.2 ppm, 62.4±0.2 ppm, 49.0±0.2 ppm, 39.1±0.2 ppm, and 21.7±0.2 ppm.
368. Compound II free form Monohydrate according to Embodiment 363, characterized by a ¹³C NMR spectrum comprising (a) a signal at 134.1±0.2 ppm and/or a signal at 21.1±0.2 ppm; and (b) signals at 74.5±0.2 ppm, 62.4±0.2 ppm, 49.0±0.2 ppm, 39.1±0.2 ppm, and 21.7±0.2 ppm.
369. Compound II free form Monohydrate according to Embodiment 363, characterized by a ¹³C NMR spectrum substantially similar to that in FIG. 68.
370. A method of preparing Compound II free form Monohydrate comprising:

humidifying Compound II free form A in 69% RH chamber equilibrating in saturated potassium iodide for 1-2 months under static conditions; and

isolating the solid.

371. Compound II free form Dihydrate.
372. Compound II free form Dihydrate according to Embodiment 363, characterized by a ¹³C NMR spectrum comprising a signal at 143.8±0.2 ppm and a signal at 38.2±0.2 ppm.
373. Compound II free form Dihydrate according to Embodiment 363, characterized by a ¹³C NMR spectrum comprising (a) one or more signals chosen from 143.8±0.2 ppm, 128.9±0.2 ppm, 126.6±0.2 ppm, 68.6±0.2 ppm, 62.7±0.2 ppm, and 37.8±0.2 ppm; and (b) one or more signals chosen from 131.8±0.2 ppm, 124.5±0.2 ppm, 124.1±0.2 ppm, 38.2±0.2 ppm, and 22.5±0.2 ppm.
374. Compound II free form Dihydrate according to Embodiment 363, characterized by a ¹³C NMR spectrum comprising (a) two or more signals chosen from 143.8±0.2 ppm, 128.9±0.2 ppm, 126.6±0.2 ppm, 68.6±0.2 ppm, 62.7±0.2 ppm, and 37.8±0.2 ppm; and (b) two or more signals chosen from 131.8±0.2 ppm, 124.5±0.2 ppm, 124.1±0.2 ppm, 38.2±0.2 ppm, and 22.5±0.2 ppm.
375. Compound II free form Dihydrate according to Embodiment 363, characterized by a ¹³C NMR spectrum comprising (a) three or more signals chosen from 143.8±0.2 ppm, 128.9±0.2 ppm, 126.6±0.2 ppm, 68.6±0.2 ppm, 62.7±0.2 ppm, and 37.8±0.2 ppm; and (b) three or more signals chosen from 131.8±0.2 ppm, 124.5±0.2 ppm, 124.1±0.2 ppm, 38.2±0.2 ppm, and 22.5±0.2 ppm.
376. Compound II free form Dihydrate according to Embodiment 363, characterized by a ¹³C NMR spectrum comprising (a) four or more signals chosen from 143.8±0.2 ppm, 128.9±0.2 ppm, 126.6±0.2 ppm, 68.6±0.2 ppm, 62.7±0.2 ppm, and 37.8±0.2 ppm; and (b) four or more signals chosen from 131.8±0.2 ppm, 124.5±0.2 ppm, 124.1±0.2 ppm, 38.2±0.2 ppm, and 22.5±0.2 ppm.
377. Compound II free form Dihydrate according to Embodiment 363, characterized by a ¹³C NMR spectrum substantially similar to that in FIG. 70.
378. A method of preparing Compound II free form Dihydrate comprising:

humidifying Compound II Neat Form A in 94% RH chamber equilibrating in saturated potassium nitrate for 12 days under static conditions; and

isolating the solid.

379. Compound II free form EtOH Solvate Form B.
380. Compound II free form EtOH Solvate Form B according to Embodiment 379, characterized by an X-ray powder diffractogram measured at ambient temperature comprising a signal at 11.6±0.2 two-theta.
381. Compound II free form EtOH Solvate Form B according to Embodiment 379, characterized by an X-ray powder diffractogram measured at ambient temperature comprising a signal at 17.1±0.2 two-theta.
382. Compound II free form EtOH Solvate Form B according to Embodiment 379, characterized by an X-ray powder diffractogram comprising a signal at 23.8±0.2 two-theta.
383. Compound II free form EtOH Solvate Form B according to Embodiments 379, characterized by an X-ray powder diffractogram comprising a signal at two or more of two-theta values 11.6±0.2, 17.1±0.2, and 23.8±0.2.
384. Compound II free form EtOH Solvate Form B according to Embodiment 379, characterized by an X-ray powder diffractogram comprising signals at 11.6±0.2 two-theta, 17.1±0.2 two-theta, and 23.8±0.2 two-theta.
385. Compound II free form EtOH Solvate Form B according to Embodiment 379, characterized by an X-ray powder diffractogram comprising (a) a signal at one or more two-theta values chosen from 11.6±0.2, 17.1±0.2, and 23.8±0.2; and (b) a signal at one or more (e.g., two, three, or four) two-theta values chosen from 7.6±0.2, 16.6±0.2, 23.3±0.2 and 23.7±0.2.
386. Compound II free form EtOH Solvate Form B according to Embodiment 379, characterized by an X-ray powder diffractogram comprising (a) a signal at two or more two-theta values chosen from 11.6±0.2, 17.1±0.2, and 23.8±0.2; and (b) a signal at one or more (e.g., two, three, or four) two-theta values chosen from 7.6±0.2, 16.6±0.2, 23.3±0.2 and 23.7±0.2.
387. Compound II free form EtOH Solvate Form B according to Embodiment 379, characterized by an X-ray powder diffractogram comprising (a) signals at 11.6±0.2 two-theta, 17.1±0.2 two-theta, and 23.8±0.2 two-theta; and (b) a signal at one or more (e.g., two, three, four, or five) two-theta values chosen from 16.6±0.2, 17.3±0.2, 18.3±0.2, 22.1±0.2, and 24.4±0.2.
388. Compound II free form EtOH Solvate Form B according to Embodiment 379, characterized by an X-ray powder diffractogram signals at 11.6±0.2 two-theta, 16.6±0.2 two-theta, 17.1±0.2 two-theta, 17.3±0.2 two-theta, 18.3±0.2 two-theta, 22.1±0.2 two-theta, 23.8±0.2 two-theta, and 24.4±0.2 two-theta.
389. Compound II free form EtOH Solvate Form B according to Embodiment 379, characterized by an X-ray powder diffractogram substantially similar to that in FIG. 71.
390. Compound II free form EtOH Solvate Form B according to any one of Embodiments 379 to 389, characterized by a TGA thermogram showing about 9% weight loss from ambient temperature up to 200° C.
391. Compound II free form EtOH Solvate Form B according to any one of Embodiments 379 to 389, characterized by a TGA thermogram substantially similar to that in FIG. 72.
392. Compound II free form EtOH Solvate Form B according to any one of Embodiments 379 to 391, characterized by a DSC curve having endothermic peaks at about 67° C. and 105° C.
393. Compound II free form EtOH Solvate Form B according to any one of Embodiments 379 to 391, characterized by a DSC curve substantially similar to that in FIG. 73.
394. A method of preparing Compound II free form EtOH Solvate Form B comprising:

slowly evaporating Compound II in EtOH at 4° C.; and

isolating the solids.

395. Compound II free form IPA Solvate.
396. Compound II free form IPA Solvate according to Embodiment 395, characterized by an X-ray powder diffractogram measured at ambient temperature comprising a signal at 8.4±0.2 two-theta.
397. Compound II free form IPA Solvate according to Embodiment 395, characterized by an X-ray powder diffractogram measured at ambient temperature comprising a signal at 11.7±0.2 two-theta.
398. Compound II free form IPA Solvate according to Embodiment 395, characterized by an X-ray powder diffractogram comprising a signal at 21.6±0.2 two-theta.
399. Compound II free form IPA Solvate according to Embodiment 395, characterized by an X-ray powder diffractogram comprising a signal at 23.3±0.2 two-theta.
400. Compound II free form IPA Solvate according to Embodiments 395, characterized by an X-ray powder diffractogram comprising a signal at two or more of two-theta values 8.4±0.2, 11.7±0.2, 21.6±0.2, and 23.3±0.2.
401. Compound II free form IPA Solvate according to Embodiments 395, characterized by an X-ray powder diffractogram comprising a signal at three or more of two-theta values 8.4±0.2, 11.7±0.2, 21.6±0.2, and 23.3±0.2.
402. Compound II free form IPA Solvate according to Embodiment 395, characterized by an X-ray powder diffractogram comprising signals at 8.4±0.2 two-theta, 11.7±0.2 two-theta, 21.6±0.2 two-theta, and 23.3±0.2 two-theta.
403. Compound II free form IPA Solvate according to Embodiment 395, characterized by an X-ray powder diffractogram comprising (a) a signal at one or more two-theta values chosen from 8.4±0.2, 11.7±0.2, 21.6±0.2, and 23.3±0.2; and (b) a signal at one or more (e.g., two, three, or four) two-theta values chosen from 17.0±0.2, 19.9±0.2, 21.9±0.2 and 22.1±0.2.
404. Compound II free form IPA Solvate according to Embodiment 395, characterized by an X-ray powder diffractogram comprising (a) a signal at two or more two-theta values chosen from 8.4±0.2, 11.7±0.2, 21.6±0.2, and 23.3±0.2; and (b) a signal at one or more (e.g., two, three, or four) two-theta values chosen from 17.0±0.2, 19.9±0.2, 21.9±0.2 and 22.1±0.2.
405. Compound II free form IPA Solvate according to Embodiment 395, characterized by an X-ray powder diffractogram comprising (a) a signal at two-theta values 8.4±0.2, 11.7±0.2, 21.6±0.2, and 23.3±0.2; and (b) a signal at one or more (e.g., two, three, or four) two-theta values chosen from 17.0±0.2, 19.9±0.2, 21.9±0.2 and 22.1±0.2.
406. Compound II free form IPA Solvate according to Embodiment 395, characterized by an X-ray powder diffractogram comprising (a) a signal at three or more two-theta values chosen from 8.4±0.2, 11.7±0.2, 21.6±0.2, and 23.3±0.2; and (b) a signal at one or more (e.g., two, three, or four) two-theta values chosen from 17.0±0.2, 19.9±0.2, 21.9±0.2 and 22.1±0.2.
407. Compound II free form IPA Solvate according to Embodiment 395, characterized by an X-ray powder diffractogram signals at 8.4±0.2 two-theta, 11.7±0.2 two-theta, 17.0±0.2 two-theta, 19.9±0.2 two-theta, 21.6±0.2 two-theta, 21.9±0.2 two-theta, 22.1±0.2 two-theta, and 23.3±0.2 two-theta.
408. Compound II free form IPA Solvate according to Embodiment 395, characterized by an X-ray powder diffractogram substantially similar to that in FIG. 74.
409. Compound II free form IPA Solvate according to any one of Embodiments 395 to 408, characterized by a ¹³C NMR spectrum comprising one or more (e.g., two, or three) signals chosen from 147.5±0.2 ppm, 74.5±0.2 ppm, and 49.5±0.2 ppm.
410. Compound II free form IPA Solvate according to any one of Embodiments 395 to 408, characterized by a ¹³C NMR spectrum comprising two signals chosen from 147.5±0.2 ppm, 74.5±0.2 ppm, and 49.5±0.2 ppm.
411. Compound II free form IPA Solvate according to any one of Embodiments 395 to 408, characterized by a ¹³C NMR spectrum comprising signals at 147.5±0.2 ppm, 74.5±0.2 ppm, and 49.5±0.2 ppm.
412. Compound II free form IPA Solvate according to any one of Embodiments 395 to 408, characterized by a ¹³C NMR spectrum comprising one or more (e.g., two, three, four, five, six, seven, eight, nine, or more) signals chosen from 147.5±0.2 ppm, 143.0±0.2 ppm, 74.9±0.2 ppm, 74.5±0.2 ppm, 61.7±0.2 ppm 49.5±0.2 ppm, 48.9±0.2 ppm, 22.4±0.2 ppm, 22.0±0.2 ppm, 21.7±0.2 ppm.
413. Compound II free form IPA Solvate according to any one of Embodiments 395 to 408, characterized by a ¹³C NMR spectrum comprising signals at 147.5±0.2 ppm, 143.0±0.2 ppm, 74.9±0.2 ppm, 74.5±0.2 ppm, 61.7±0.2 ppm, 49.5±0.2 ppm, 48.9±0.2 ppm, 22.4±0.2 ppm, 22.0±0.2 ppm, 21.7±0.2 ppm.
414. Compound II free form IPA Solvate according to any one of Embodiments 395 to 408, characterized by a ¹³C NMR spectrum substantially similar to that in FIG. 75.
415. A method of preparing Compound II free form IPA Solvate comprising:

making a slurry of Compound II free form Hemihydrate Form A in 50/50 IPA/heptane (vol/vol);

shaking overnight in a shaker block at 20° C. and 1000 rpm; and

isolating the solids.

416. Compound II free form MEK Solvate.
417. Compound II free form MEK Solvate according to Embodiment 416, characterized by a ¹³C NMR spectrum comprising one or more (e.g., two, three, four, five, six, or more) signals chosen from 8.2±0.2 ppm, 23.2±0.2 ppm, 30.0±0.2 ppm, 35.0±0.2 ppm, 35.7±0.2 ppm 39.3±0.2 ppm, and 63.3±0.2 ppm.
418. Compound II free form MEK Solvate according to Embodiment 416, characterized by a ¹³C NMR spectrum comprising signals at 8.2±0.2 ppm, 23.2±0.2 ppm, 30.0±0.2 ppm, 35.0±0.2 ppm, 35.7±0.2 ppm 39.3±0.2 ppm, and 63.3±0.2 ppm.
419. Compound II free form MEK Solvate according to Embodiment 416, characterized by a ¹³C NMR spectrum substantially similar to that in FIG. 76.
420. A method of preparing Compound II free form MEK Solvate comprising:

charging Compound II free form Hemihydrate Form A to a jacketed reactor and adding methyl ethyl ketone;

agitating at 300 rpm in a reactor at 45° C.;

adding Compound II free form Hemihydrate Form A as seeds and holding at 45° C. for 30 minutes;

cooling to 20° C. for 1 hour; and

isolating the solids.

421. Compound II free form MeOH Solvate.
422. Compound II free form MeOH Solvate according to Embodiment 421, characterized by an X-ray powder diffractogram measured at ambient temperature comprising a signal at 13.4±0.2 two-theta.
423. Compound II free form MeOH Solvate according to Embodiment 421, characterized by an X-ray powder diffractogram measured at ambient temperature comprising a signal at 16.6±0.2 two-theta.
424. Compound II free form MeOH Solvate according to 421, characterized by an X-ray powder diffractogram comprising a signal at 24.3±0.2 two-theta.
425. Compound II free form MeOH Solvate according to 252, characterized by an X-ray powder diffractogram comprising a signal at 24.4±0.2 two-theta.
426a. Compound II free form MeOH Solvate according to 421, characterized by an X-ray powder diffractogram comprising a signal at 26.3±0.2 two-theta.
426b. Compound II free form MeOH Solvate according to Embodiment 421, characterized by an X-ray powder diffractogram comprising signals at two or more two-theta values chosen from 13.4±0.2, 16.6±0.2, 24.3±0.2, 24.4±0.2, and 26.3±0.2.
427. Compound II free form MeOH Solvate according to Embodiment 421, characterized by an X-ray powder diffractogram comprising signals at three or more two-theta values chosen from 13.4±0.2, 16.6±0.2, 24.3±0.2, 24.4±0.2, and 26.3±0.2.
428. Compound II free form MeOH Solvate according to Embodiment 421, characterized by an X-ray powder diffractogram comprising signals at four or more two-theta values chosen from 13.4±0.2, 16.6±0.2, 24.3±0.2, 24.4±0.2, and 26.3±0.2.
429. Compound II free form MeOH Solvate according to Embodiment 421, characterized by an X-ray powder diffractogram comprising signals at 13.4±0.2 two-theta, 16.6±0.2 two-theta, 24.3±0.2 two-theta, 24.4±0.2 two-theta, and 26.3±0.2 two-theta.
430. Compound II free form MeOH Solvate according to Embodiment 421, characterized by an X-ray powder diffractogram comprising (a) a signal at two or more two-theta values chosen from 13.4±0.2, 16.6±0.2, 24.3±0.2, 24.4±0.2, and 26.3±0.2; and (b) a signal at one or more (e.g., two, three, or four) two-theta values chosen from 12.0±0.2, 21.2±0.2, 24.1±0.2, and 24.2±0.2.
431. Compound II free form MeOH Solvate according to Embodiment 421, characterized by an X-ray powder diffractogram comprising (a) a signal at three or more two-theta values chosen from 13.4±0.2, 16.6±0.2, 24.3±0.2, 24.4±0.2, and 26.3±0.2; and (b) a signal at one or more (e.g., two, three, or four) two-theta values chosen from 12.0±0.2, 21.2±0.2, 24.1±0.2, and 24.2±0.2.
432. Compound II free form MeOH Solvate according to Embodiment 421, characterized by an X-ray powder diffractogram comprising (a) signals at 13.4±0.2 two-theta, 16.6±0.2 two-theta, 24.3±0.2 two-theta, 24.4±0.2 two-theta, and 26.3±0.2 two-theta; and (b) a signal at one or more (e.g., two, three, or four) two-theta values chosen from 12.0±0.2, 21.2±0.2, 24.1±0.2, and 24.2±0.2.
433. Compound II free form MeOH Solvate according to Embodiment 421, characterized by an X-ray powder diffractogram comprising signals at 12.0±0.2 two-theta, 13.4±0.2 two-theta, 16.6±0.2 two-theta, 21.2±0.2 two-theta, 24.1±0.2 two-theta, and 24.2±0.2, 24.3±0.2 two-theta, 24.4±0.2 two-theta.
434. Compound II free form MeOH Solvate according to Embodiment 421, characterized by an X-ray powder diffractogram substantially similar to that in FIG. 77.
435. Compound II free form MeOH Solvate according to any one of Embodiments 421 to 434, characterized by a TGA thermogram showing 0.87% weight loss from ambient temperature up to 150° C.
436. Compound II free form MeOH Solvate according to any one of Embodiments 421 to 434, characterized by a TGA thermogram substantially similar to that in FIG. 79.
437. Compound II free form MeOH Solvate according to any one of Embodiments 421 to 436, characterized by a DSC curve having endothermic peaks at about 79° C., 112° C., and 266° C.
438. Compound II free form MeOH Solvate according to any one of Embodiments 421 to 436, characterized by a DSC curve substantially similar to that in FIG. 80.
439. Compound II free form MeOH Solvate according to any one of Embodiments 421 to 438, characterized by a ¹³C NMR spectrum comprising one or more (e.g., two, three, four, five, or six) signals chosen from 133.6±0.2 ppm, 74.8±0.2 ppm, 67.7±0.2 ppm, 62.6±0.2 ppm, 49.8±0.2 ppm, and 21.2±0.2 ppm.
440. Compound II free form MeOH Solvate according to any one of Embodiments 421 to 438, characterized by a ¹³C NMR spectrum comprising signals at 133.6±0.2 ppm, 74.8±0.2 ppm, 67.7±0.2 ppm, 62.6±0.2 ppm, 49.8±0.2 ppm, and 21.2±0.2 ppm.
441. Compound II free form MeOH Solvate according to any one of Embodiments 421 to 438, characterized by a ¹³C NMR spectrum substantially similar to that in FIG. 78.
442. Compound II free form MeOH Solvate according to any one of Embodiments 421 to 441, having a single crystal unit cell characterized by a monoclinic crystal system, a C2 space group, and unit cell dimensions measured at 100 K on a Bruker diffractometer equipped Cu Kα radiation (λ=1.54178 Å) of:

| | | | |
|---|---|---|---|
| a | 22.2 ± 0.1 Å | α | 90° |
| b | 7.8 ± 0.1 Å | β | 114.5 ± 0.1° |
| c | 11.9 ± 0.1 Å | γ | 90°. |

443. A method of preparing Compound II free form MeOH Solvate comprising:

mixing Amorphous free form Compound II with MeOH followed by rotary evaporation; and

isolating Compound II free form MeOH Solvate.

### 444. Compound II Phosphate Salt, Acetone Solvate Form A.

445. Compound II Phosphate Salt Acetone Solvate Form A according to Embodiment 444, characterized by an X-ray powder diffractogram measured at ambient temperature comprising a signal at 8.7±0.2 two-theta.
446. Compound II Phosphate Salt Acetone Solvate Form A according to Embodiment 444, characterized by an X-ray powder diffractogram measured at ambient temperature comprising a signal at 9.4±0.2 two-theta.
447. Compound II Phosphate Salt Acetone Solvate Form A according to 444, characterized by an X-ray powder diffractogram comprising a signal at 15.0±0.2 two-theta.
448. Compound II Phosphate Salt Acetone Solvate Form A according to 444, characterized by an X-ray powder diffractogram comprising a signal at 18.4±0.2 two-theta.
449. Compound II Phosphate Salt Acetone Solvate Form A according to 444, characterized by an X-ray powder diffractogram comprising a signal at 26.3±0.2 two-theta.
450. Compound II Phosphate Salt Acetone Solvate Form A according to Embodiment 444, characterized by an X-ray powder diffractogram comprising signals at two or more two-theta values chosen from 8.7±0.2, 9.4±0.2, 15.0±0.2, and 18.4±0.2.
451. Compound II Phosphate Salt Acetone Solvate Form A according to Embodiment 444, characterized by an X-ray powder diffractogram comprising signals at three or more two-theta values chosen from 8.7±0.2, 9.4±0.2, 15.0±0.2, and 18.4±0.2.
452. Compound II Phosphate Salt Acetone Solvate Form A according to Embodiment 444, characterized by an X-ray powder diffractogram comprising signals at 8.7±0.2 two-theta, 9.4±0.2 two-theta, 15.0±0.2 two-theta, and 18.4±0.2 two-theta.
453a. Compound II Phosphate Salt Acetone Solvate Form A according to Embodiment 444, characterized by an X-ray powder diffractogram comprising (a) signals at 8.7±0.2 two-theta, 9.4±0.2 two-theta, 15.0±0.2 two-theta, and 18.4±0.2 two-theta; and (b) a signal at one or more two-theta values chosen from 10.4±0.2, 18.8±0.2, 20.8±0.2, and 22.6±0.2.
453b. Compound II Phosphate Salt Acetone Solvate Form A according to Embodiment 444, characterized by an X-ray powder diffractogram comprising (a) signals at 8.7±0.2 two-theta, 9.4±0.2 two-theta, 15.0±0.2 two-theta, and 18.4±0.2 two-theta; and (b) signals at two or more two-theta values chosen from 10.4±0.2, 18.8±0.2, 20.8±0.2, and 22.6±0.2.
454. Compound II Phosphate Salt Acetone Solvate Form A according to Embodiment 444, characterized by an X-ray powder diffractogram comprising (a) a signal at 8.7±0.2 two-theta, 9.4±0.2 two-theta, 15.0±0.2 two-theta, and 18.4±0.2 two-theta; and (b) signals at three or more (e.g., two, three, or four) two-theta values chosen from 10.4±0.2, 18.8±0.2, 20.8±0.2, and 22.6±0.2.
455. Compound II Phosphate Salt Acetone Solvate Form A according to Embodiment 444, characterized by an X-ray powder diffractogram comprising signals at 8.7±0.2 two-theta, 9.4±0.2 two-theta, 10.4±0.2 two-theta, 15.0±0.2 two-theta, 18.4±0.2 two-theta 18.8±0.2 two-theta, 20.8±0.2 two-theta, and 22.6±0.2 two-theta.
456. Compound II Phosphate Salt Acetone Solvate Form A according to Embodiment 444, characterized by an X-ray powder diffractogram substantially similar to that in FIG. 85.
457. Compound II Phosphate Salt Acetone Solvate Form A according to any one of Embodiments 444 to 456, characterized by a TGA thermogram showing 0.9% weight loss from ambient temperature up to 200° C.
458. Compound II Phosphate Salt Acetone Solvate Form A according to any one of Embodiments 444 to 456, characterized by a TGA thermogram substantially similar to that in FIG. 87.
459. Compound II Phosphate Salt Acetone Solvate Form A according to any one of Embodiments 444 to 458, characterized by a DSC curve having an endothermic peak at about 242° C.
460. Compound II Phosphate Salt Acetone Solvate Form A according to any one of Embodiments 444 to 458, characterized by a DSC curve substantially similar to that in FIG. 88.
461. Compound II Phosphate Salt Acetone Solvate Form A according to any one of Embodiments 444 to 460, characterized by a ¹³C NMR spectrum comprising one or more (e.g., two, three, four, five, six, seven, eight, nine, or ten) signals chosen from 142.3±0.2 ppm, 126.3±0.2 ppm, 73.0±0.2 ppm, 72.3±0.2 ppm, 64.8±0.2 ppm, 62.0±0.2 ppm, 49.4±0.2 ppm, 47.9±0.2 ppm, and 38.2±0.2 ppm.
462. Compound II Phosphate Salt Acetone Solvate Form A according to any one of Embodiments 444 to 460, characterized by a ¹³C NMR spectrum comprising signals at 142.3±0.2 ppm, 126.3±0.2 ppm, 73.0±0.2 ppm, 72.3±0.2 ppm, 64.8±0.2 ppm, 62.0±0.2 ppm, 49.4±0.2 ppm, 47.9±0.2 ppm, and 38.2±0.2 ppm.
463. Compound II Phosphate Salt Acetone Solvate Form A according to any one of Embodiments 444 to 460, characterized by a ¹³C NMR spectrum substantially similar to that in FIG. 86.
464. A method of preparing Compound II Phosphate Salt Acetone Solvate Form A comprising:

combining Compound II Phosphate Salt Hemihydrate Form A to a mixture of acetone and water,

stirring at ambient temperature for three days, and

isolating the solids.

465. A method of preparing Compound II Phosphate Salt Acetone Solvate Form A comprising:

adding Compound II Phosphate Salt Hemihydrate Form A to a mixture of acetone and water at room temperature to form a suspension;

stirring overnight and filtering to obtain a clear saturated solution;

adding equal amounts of Compound II Phosphate Salt Hemihydrate Form A and Compound II Phosphate Salt Form C to the saturated solution;

stirring at ambient temperature for 4 days; and

isolating the solids.

### 466. Compound II Phosphate Salt Form A.

467. Compound II Phosphate Salt Form A according to Embodiment 466, characterized by an X-ray powder diffractogram measured at ambient temperature comprising a signal at 7.0±0.2 two-theta.
468. Compound II Phosphate Salt Form A according to Embodiment 466, characterized by an X-ray powder diffractogram measured at ambient temperature comprising a signal at 9.9±0.2 two-theta.
469. Compound II Phosphate Salt Form A according to 466, characterized by an X-ray powder diffractogram comprising a signal at 14.1±0.2 two-theta.
470. Compound II Phosphate Salt Form A according to 466, characterized by an X-ray powder diffractogram comprising a signal at 17.5±0.2 two-theta.
471. Compound II Phosphate Salt Form A according to 466, characterized by an X-ray powder diffractogram comprising a signal at 19.9±0.2 two-theta.
472. Compound II Phosphate Salt Form A according to Embodiment 466, characterized by an X-ray powder diffractogram comprising signals at two or more two-theta values chosen from 7.0±0.2, 9.9±0.2, 14.1±0.2, 17.5±0.2 and 19.9±0.2.
473. Compound II Phosphate Salt Form A according to Embodiment 466, characterized by an X-ray powder diffractogram comprising signals at three or more two-theta values chosen from 7.0±0.2, 9.9±0.2, 14.1±0.2, 17.5±0.2 and 19.9±0.2.
474. Compound II Phosphate Salt Form A according to Embodiment 466, characterized by an X-ray powder diffractogram comprising signals at four or more two-theta values chosen from 7.0±0.2, 9.9±0.2, 14.1±0.2, 17.5±0.2 and 19.9±0.2.
475. Compound II Phosphate Salt Form A according to Embodiment 466, characterized by an X-ray powder diffractogram comprising signals at 7.0±0.2 two-theta, 9.9±0.2 two-theta, 14.1±0.2 two-theta, 17.5±0.2 two-theta, and 19.9±0.2 two-theta.
476. Compound II Phosphate Salt Form A according to Embodiment 466, characterized by an X-ray powder diffractogram comprising (a) a signal at one or more (e.g., two, three, four, or five) two-theta values chosen from 7.0±0.2, 9.9±0.2, 14.1±0.2, 17.5±0.2 and 19.9±0.2; and (b) a signal at one or more two-theta values chosen from 8.9±0.2, 16.9±0.2, 18.5±0.2, and 21.6±0.2.
477. Compound II Phosphate Salt Form A according to Embodiment 466, characterized by an X-ray powder diffractogram comprising (a) a signal at one or more (e.g., two, three, four, or five) two-theta values chosen from 7.0±0.2, 9.9±0.2, 14.1±0.2, 17.5±0.2 and 19.9±0.2; and (b) a signal at two or more two-theta values chosen from 8.9±0.2, 16.9±0.2, 18.5±0.2, and 21.6±0.2.
478. Compound II Phosphate Salt Form A according to Embodiment 466, characterized by an X-ray powder diffractogram comprising (a) a signal at 7.0±0.2, 9.9±0.2, 14.1±0.2, 17.5±0.2 and 19.9±0.2; and (b) a signal at three or more (e.g., two, three, or four) two-theta values chosen from 8.9±0.2, 16.9±0.2, 18.5±0.2, and 21.6±0.2.
479. Compound II Phosphate Salt Form A according to Embodiment 466, characterized by an X-ray powder diffractogram comprising signals at 7.0±0.2 two-theta, 8.9±0.2, 9.9±0.2 two-theta, 14.1±0.2 two-theta, 16.9±0.2, 17.5±0.2 two-theta, 18.5±0.2, 19.9±0.2 two-theta, and 21.6±0.2 two-theta.
480. Compound II Phosphate Salt Form A according to Embodiment 466, characterized by an X-ray powder diffractogram substantially similar to that in FIG. 89.
481. Compound II Phosphate Salt Form A according to any one of Embodiments 466 to 480, characterized by a TGA thermogram showing negligible weight loss from ambient temperature up to 200° C.
482. Compound II Phosphate Salt Form A according to any one of Embodiments 466 to 480, characterized by a TGA thermogram substantially similar to that in FIG. 92.
483. Compound II Phosphate Salt Form A according to any one of Embodiments 466 to 482, characterized by a DSC curve having endothermic peaks at about 228° C. and 237° C.
484. Compound II Phosphate Salt Form A according to any one of Embodiments 466 to 482, characterized by a DSC curve substantially similar to that in FIG. 93.
485. Compound II Phosphate Salt Form A according to any one of Embodiments 466 to 484, characterized by a ¹³C NMR spectrum comprising one or more signals chosen from 72.1±0.2 ppm, 62.0±0.2 ppm, 49.4±0.2 ppm, and 17.5±0.2 ppm.
486. Compound II Phosphate Salt Form A according to any one of Embodiments 466 to 484, characterized by a ¹³C NMR spectrum comprising two or more signals chosen from 72.1±0.2 ppm, 62.0±0.2 ppm, 49.4±0.2 ppm, and 17.5±0.2 ppm.
487. Compound II Phosphate Salt Form A according to any one of Embodiments 466 to 484, characterized by a ¹³C NMR spectrum comprising three or more signals chosen from 72.1±0.2 ppm, 62.0±0.2 ppm, 49.4±0.2 ppm, and 17.5±0.2 ppm.
488. Compound II Phosphate Salt Form A according to any one of Embodiments 466 to 484, characterized by a ¹³C NMR spectrum comprising signals at 72.1±0.2 ppm, 62.0±0.2 ppm, 49.4±0.2 ppm, and 17.5±0.2 ppm.
489. Compound II Phosphate Salt Form A according to any one of Embodiments 466 to 484, characterized by a ¹³C NMR spectrum comprising (a) one or more signals chosen from 72.1±0.2 ppm, 62.0±0.2 ppm, 49.4±0.2 ppm, and 17.5±0.2 ppm; and (b) one or more signals chosen from 72.9±0.2 ppm, 64.4±0.2 ppm, and 64.1±0.2 ppm.
490. Compound II Phosphate Salt Form A according to any one of Embodiments 466 to 484, characterized by a ¹³C NMR spectrum comprising (a) one or more signals chosen from 72.1±0.2 ppm, 62.0±0.2 ppm, 49.4±0.2 ppm, and 17.5±0.2 ppm; and (b) signals at 72.9±0.2 ppm, 64.4±0.2 ppm, and 64.1±0.2 ppm.
491. Compound II Phosphate Salt Form A according to any one of Embodiments 466 to 484, characterized by a ¹³C NMR spectrum comprising signals at 72.9±0.2 ppm, 72.1±0.2 ppm, 64.4±0.2 ppm, 64.1±0.2 ppm, 62.0±0.2 ppm, and 49.4±0.2 ppm, and 17.5±0.2 ppm.
492. Compound II Phosphate Salt Form A according to any one of Embodiments 466 to 484, characterized by a ¹³C NMR spectrum substantially similar to that in FIG. 90.
493. Compound II Phosphate Salt Form A according to any one of Embodiments 466 to 492, characterized by a ³¹P CPMAS spectrum comprising one or more signals chosen from 3.3±0.2 ppm, 2.2±0.2 ppm, and −0.4±0.2 ppm.
494. Compound II Phosphate Salt Form A according to any one of Embodiments 466 to 492, characterized by a ³¹P CPMAS spectrum comprising signals at 3.3±0.2 ppm, 2.2±0.2 ppm, and −0.4±0.2 ppm.
495. Compound II Phosphate Salt Form A according to any one of Embodiments 466 to 492, characterized by a ³¹P CPMAS spectrum substantially similar to that in FIG. 91.
496. A method of preparing Compound II Phosphate Salt Form A comprising:

adding MEK followed by phosphoric acid to Amorphous free form Compound II;

stirring at ambient temperature for 48 hours;

filtering and washing the solids with 4:1 n-heptane/MEK (v/v);

drying in a vacuum oven 18 hours at 60° C.; and

isolating the solids. 497. Compound II Phosphate Salt Form C.

498. Compound II Phosphate Salt Form C according to Embodiment 497, characterized by an X-ray powder diffractogram measured at ambient temperature comprising a signal at 13.5±0.2 two-theta.
499. Compound II Phosphate Salt Form C according to Embodiment 497, characterized by an X-ray powder diffractogram measured at ambient temperature comprising a signal at 13.7±0.2 two-theta.
500. Compound II Phosphate Salt Form C according to Embodiment 497, characterized by an X-ray powder diffractogram comprising a signal at 15.0±0.2 two-theta.
501. Compound II Phosphate Salt Form C according to Embodiment 497, characterized by an X-ray powder diffractogram comprising signals at two or more two-theta values chosen from 13.5±0.2, 13.7±0.2, and 15.0±0.2.
502. Compound II Phosphate Salt Form C according to Embodiment 497, characterized by an X-ray powder diffractogram comprising signals at 13.5±0.2 two-theta, 13.7±0.2 two-theta, and 15.0±0.2 two-theta.
503. Compound II Phosphate Salt Form C according to Embodiment 497, characterized by an X-ray powder diffractogram comprising (a) signals at 13.5±0.2 two-theta, 13.7±0.2 two-theta, and 15.0±0.2 two-theta; and (b) a signal at one or more two-theta values chosen from 9.1±0.2, 9.4±0.2, 10.4±0.2, 11.0±0.2, and 18.6±0.2.
504. Compound II Phosphate Salt Form C according to Embodiment 497, characterized by an X-ray powder diffractogram comprising (a) signals at 13.5±0.2 two-theta, 13.7±0.2 two-theta, and 15.0±0.2 two-theta; and (b) a signal at two or more two-theta values chosen from 9.1±0.2, 9.4±0.2, 10.4±0.2, 11.0±0.2, and 18.6±0.2.
505. Compound II Phosphate Salt Form C according to Embodiment 497, characterized by an X-ray powder diffractogram comprising (a) signals at 13.5±0.2 two-theta, 13.7±0.2 two-theta, and 15.0±0.2 two-theta; and (b) a signal at three or more two-theta values chosen from 9.1±0.2, 9.4±0.2, 10.4±0.2, 11.0±0.2, and 18.6±0.2.
506. Compound II Phosphate Salt Form C according to Embodiment 497, characterized by an X-ray powder diffractogram comprising signals at 9.1±0.2 two-theta, 9.4±0.2 two-theta, 10.4±0.2 two-theta, 11.0±0.2 two-theta, 13.5±0.2±0.2 two-theta, 13.7±0.2 two-theta, 15.0±0.2 two-theta, and 18.6±0.2 two-theta.
507. Compound II Phosphate Salt Form C according to Embodiment 497, characterized by an X-ray powder diffractogram substantially similar to that in FIG. 94.
508. Compound II Phosphate Salt Form C according to any one of Embodiments 497 to 507, characterized by a TGA thermogram showing 1.6% weight loss from ambient temperature up to 150° C.
509. Compound II Phosphate Salt Form C according to any one of Embodiments 497 to 507, characterized by a TGA thermogram substantially similar to that in FIG. 96.
510. Compound II Phosphate Salt Form C according to any one of Embodiments 497 to 509, characterized by a DSC curve having an endothermic peak at about 244° C.
511. Compound II Phosphate Salt Form C according to any one of Embodiments 497 to 509, characterized by a DSC curve substantially similar to that in FIG. 97.
512. Compound II Phosphate Salt Form C according to any one of Embodiments 497 to 511, characterized by a ¹³C NMR spectrum comprising one or more signals chosen from 139.0±0.2 ppm, 127.8±0.2 ppm, 66.5±0.2 ppm, 62.5±0.2 ppm, and 16.8±0.2 ppm.
513. Compound II Phosphate Salt Form C according to any one of Embodiments 497 to 511, characterized by a ¹³C NMR spectrum comprising two or more signals chosen from 139.0±0.2 ppm, 127.8±0.2 ppm, 66.5±0.2 ppm, 62.5±0.2 ppm, and 16.8±0.2 ppm.
514. Compound II Phosphate Salt Form C according to any one of Embodiments 497 to 511, characterized by a ¹³C NMR spectrum comprising three or more signals chosen from 139.0±0.2 ppm, 127.8±0.2 ppm, 66.5±0.2 ppm, 62.5±0.2 ppm, and 16.8±0.2 ppm.
515. Compound II Phosphate Salt Form C according to any one of Embodiments 497 to 511, characterized by a ¹³C NMR spectrum comprising four or more signals chosen from 139.0±0.2 ppm, 127.8±0.2 ppm, 66.5±0.2 ppm, 62.5±0.2 ppm, and 16.8±0.2 ppm.
516. Compound II Phosphate Salt Form C according to any one of Embodiments 497 to 511, characterized by a ¹³C NMR spectrum comprising signals at 139.0±0.2 ppm, 127.8±0.2 ppm, 66.5±0.2 ppm, 62.5±0.2 ppm, and 16.8±0.2 ppm.
517. Compound II Phosphate Salt Form C according to any one of Embodiments 497 to 511, characterized by a ¹³C NMR spectrum comprising (a) one or more signals chosen from 139.0±0.2 ppm, 127.8±0.2 ppm, 66.5±0.2 ppm, 62.5±0.2 ppm, and 16.8±0.2 ppm; and (b) one or more (e.g., two, three, four, or five) signals chosen from 143.0±0.2 ppm, 140.3±0.2 ppm, 139.6±0.2 ppm, 72.7±0.2 ppm, 64.1±0.2 ppm, and 47.7±0.2 ppm.
518. Compound II Phosphate Salt Form C according to any one of Embodiments 497 to 511, characterized by a ¹³C NMR spectrum comprising (a) two or more signals chosen from 139.0±0.2 ppm, 127.8±0.2 ppm, 66.5±0.2 ppm, 62.5±0.2 ppm, and 16.8±0.2 ppm; and (b) one or more (e.g., two, three, four, or five) signals chosen from 143.0±0.2 ppm, 140.3±0.2 ppm, 139.6±0.2 ppm, 72.7±0.2 ppm, 64.1±0.2 ppm, and 47.7±0.2 ppm.
519. Compound II Phosphate Salt Form C according to any one of Embodiments 497 to 511, characterized by a ¹³C NMR spectrum comprising (a) signals at 139.0±0.2 ppm, 127.8±0.2 ppm, 66.5±0.2 ppm, 62.5±0.2 ppm, and 16.8±0.2 ppm; and (b) one or more (e.g., two, three, four, or five) signals chosen from 143.0±0.2 ppm, 140.3±0.2 ppm, 139.6±0.2 ppm, 72.7±0.2 ppm, 64.1±0.2 ppm, and 47.7±0.2 ppm.
520. Compound II Phosphate Salt Form C according to any one of Embodiments 497 to 511, characterized by a ¹³C NMR spectrum substantially similar to that in FIG. 95.
521. A method of preparing Compound II Phosphate Salt Form C comprising:

preparing a slurry of Compound II Phosphate Salt Hemihydrate Form A in 1-butanol at 80° C.; and

centrifuging slurry to isolate the solids.

### 522. Compound I Maleate (Salt or Co-Crystal) Form A.

523. Compound I Maleate (Salt or Co-Crystal) Form A according to Embodiment 522, characterized by an X-ray powder diffractogram comprising a signal at 27.6±0.2 two-theta.
524. Compound I Maleate (Salt or Co-Crystal) Form A according to Embodiment 522, characterized by an X-ray powder diffractogram comprising signals at signal at 27.6±0.2 two-theta and 20.0±0.2 two-theta.
525. Compound I Maleate (Salt or Co-Crystal) Form A according to Embodiment 522, characterized by an X-ray powder diffractogram comprising signals at 8.6±0.2, 19.9±0.2, and 28.3±0.2 two-theta.
526. Compound I Maleate (Salt or Co-Crystal) Form A according to Embodiment 522, characterized by an X-ray powder diffractogram comprising (a) a signal at 27.6±0.2 two-theta and (b) a signal at one or more two-theta values chosen from 13.7±0.2, 14.5±0.2, 15.5±0.2, 18.3±0.2, and 20.0±0.2.
527. Compound I Maleate (Salt or Co-Crystal) Form A according to Embodiment 522, characterized by an X-ray powder diffractogram comprising (a) a signal at 27.6±0.2 two-theta and (b) signals at two or more two-theta values chosen from 13.7±0.2, 14.5±0.2, 15.5±0.2, 18.3±0.2, and 20.0±0.2.
528. Compound I Maleate (Salt or Co-Crystal) Form A according to Embodiment 522, characterized by an X-ray powder diffractogram comprising (a) a signal at 27.6±0.2 two-theta and (b) signals at three or more two-theta values chosen from 13.7±0.2, 14.5±0.2, 15.5±0.2, 18.3±0.2, and 20.0±0.2.
529. Compound I Maleate (Salt or Co-Crystal) Form A according to Embodiment 522, characterized by an X-ray powder diffractogram comprising (a) a signal at 27.6±0.2 two-theta and (b) signals at four or more two-theta values chosen from 13.7±0.2, 14.5±0.2, 15.5±0.2, 18.3±0.2, and 20.0±0.2.
530. Compound I Maleate (Salt or Co-Crystal) Form A according to Embodiment 522, characterized by an X-ray powder diffractogram comprising signals at 27.6±0.2 two-theta, 13.7±0.2 two-theta, 14.5±0.2 two-theta, 15.5±0.2 two-theta, 18.3±0.2 two-theta, and 20.0±0.2 two-theta.
531. Compound I Maleate (Salt or Co-Crystal) Form A according to Embodiment 522, characterized by an X-ray powder diffractogram substantially similar to that in FIG. 39.
532. Compound I Maleate (Salt or Co-Crystal) Form A according to any one of Embodiments 522 to 531, characterized by a TGA thermogram showing minimal weight loss until degradation.
533. Compound I Maleate (Salt or Co-Crystal) Form A according to any one of Embodiments 522 to 531, characterized by a TGA thermogram substantially similar to that in FIG. 40.
534. Compound I Maleate (Salt or Co-Crystal) Form A according to any one of Embodiments 522 to 533, characterized by a DSC curve having an endothermic peak at about 201° C.
535. Compound I Maleate (Salt or Co-Crystal) Form A according to any one of Embodiments 522 to 531, characterized by a DSC curve substantially similar to that in FIG. 41.
536. A method of preparing Compound I Maleate (Salt or Co-Crystal) Form A comprising:

dissolving Compound I Monohydrate in acetonitrile;

adding maleic acid to form a suspension and stirring at ambient temperature for 3 days;

centrifuging the suspension and air drying the resulting wet cake; and

isolating the solids.

### 537. Compound I Maleate (Salt or Co-Crystal) Form B.

538. Compound I Maleate (Salt or Co-Crystal) Form B according to Embodiment 537, characterized by an X-ray powder diffractogram comprising a signal at 4.9 two-theta.
539. Compound I Maleate (Salt or Co-Crystal) Form B according to Embodiment 537, characterized by an X-ray powder diffractogram comprising a signal at 26.0 two-theta.
540. Compound I Maleate (Salt or Co-Crystal) Form B according to Embodiment 537, characterized by an X-ray powder diffractogram comprising signals at signal at 4.9±0.2 two-theta and 26.0±0.2 two-theta.
541. Compound I Maleate (Salt or Co-Crystal) Form B according to Embodiment 522, characterized by an X-ray powder diffractogram comprising (a) a signal at 4.9±0.2 two-theta and/or a signal at 26.0±0.2 two-theta; and (b) a signal at one or more two-theta values chosen from 13.8±0.2, 14.7±0.2, 15.4±0.2, 18.3±0.2, and 19.6±0.2.
542. Compound I Maleate (Salt or Co-Crystal) Form B according to Embodiment 537, characterized by an X-ray powder diffractogram comprising (a) a signal at 4.9±0.2 two-theta and/or a signal at 26.0±0.2 two-theta; and (b) signals at two or more two-theta values chosen from 13.8±0.2, 14.7±0.2, 15.4±0.2, 18.3±0.2, and 19.6±0.2.
543. Compound I Maleate (Salt or Co-Crystal) Form B according to Embodiment 522, characterized by an X-ray powder diffractogram comprising (a) a signal at 4.9±0.2 two-theta and/or a signal at 26.0±0.2 two-theta; and (b) signals at three or more two-theta values chosen from 13.8±0.2, 14.7±0.2, 15.4±0.2, 18.3±0.2, and 19.6±0.2.
544. Compound I Maleate (Salt or Co-Crystal) Form B according to Embodiment 537, characterized by an X-ray powder diffractogram comprising (a) a signal at 4.9±0.2 two-theta and/or a signal at 26.0±0.2 two-theta; and (b) signals at four or more two-theta values chosen from 13.8±0.2, 14.7±0.2, 15.4±0.2, 18.3±0.2, and 19.6±0.2.
545. Compound I Maleate (Salt or Co-Crystal) Form B according to Embodiment 537, characterized by an X-ray powder diffractogram comprising signals at 4.9±0.2 two-theta, 13.8±0.2 two-theta, 14.7±0.2 two-theta, 15.4±0.2 two-theta, 18.3±0.2 two-theta, 19.6±0.2 two-theta, and 26.0±0.2 two-theta.
546. Compound I Maleate (Salt or Co-Crystal) Form B according to Embodiment 537, characterized by an X-ray powder diffractogram substantially similar to that in FIG. 42.
547. Compound I Maleate (Salt or Co-Crystal) Form B according to any one of Embodiments 537 to 546, characterized by a TGA thermogram showing minimal weight loss until degradation.
548. Compound I Maleate (Salt or Co-Crystal) Form B according to any one of Embodiments 537 to 546, characterized by a TGA thermogram substantially similar to that in FIG. 43.
549. Compound I Maleate (Salt or Co-Crystal) Form B according to any one of Embodiments 537 to 548, characterized by a DSC curve having an endothermic peak at about 206° C.
550. Compound I Maleate (Salt or Co-Crystal) Form B according to any one of Embodiments 537 to 548, characterized by a DSC curve substantially similar to that in FIG. 44.
551. A method of preparing Compound I Maleate (Salt or Co-Crystal) Form B comprising:

dissolving Compound I Monohydrate in ethanol;

adding maleic acid and stirring at ambient temperature for 3 days;

fast evaporating for 5 days; and

isolating the solids.

### 552. Compound I Fumaric Acid (Salt or Co-Crystal) Form A.

553. Compound I Fumaric Acid (Salt or Co-Crystal) Form A according to Embodiment 552, characterized by an X-ray powder diffractogram comprising a signal at 21.5 two-theta.
554. Compound I Fumaric Acid (Salt or Co-Crystal) Form A according to Embodiment 552, characterized by an X-ray powder diffractogram comprising a signal at 14.4 two-theta.
555. Compound I Fumaric Acid (Salt or Co-Crystal) Form A according to Embodiment 552, characterized by an X-ray powder diffractogram comprising a signal at 16.9 two-theta.
556. Compound I Fumaric Acid (Salt or Co-Crystal) Form A according to Embodiment 552, characterized by an X-ray powder diffractogram comprising a signal at 20.7 two-theta.
557. Compound I Fumaric Acid (Salt or Co-Crystal) Form A according to Embodiment 552, characterized by an X-ray powder diffractogram comprising signals at two or more two-theta values chosen from 14.4±0.2, 14.6±0.2, 16.9±0.2, 20.7±0.2, 20.9±0.2, and 21.5±0.2.
558. Compound I Fumaric Acid (Salt or Co-Crystal) Form A according to Embodiment 552, characterized by an X-ray powder diffractogram comprising signals at three or more two-theta values chosen from 14.4±0.2, 14.6±0.2, 16.9±0.2, 20.7±0.2, 20.9±0.2, and 21.5±0.2.
559. Compound I Fumaric Acid (Salt or Co-Crystal) Form A according to Embodiment 522, characterized by an X-ray powder diffractogram comprising signals at four or more two-theta values chosen from 14.4±0.2, 14.6±0.2, 16.9±0.2, 20.7±0.2, 20.9±0.2, and 21.5±0.2.
560. Compound I Fumaric Acid (Salt or Co-Crystal) Form A according to Embodiment 552, characterized by an X-ray powder diffractogram comprising signals at five or more two-theta values chosen from 14.4±0.2, 14.6±0.2, 16.9±0.2, 20.7±0.2, 20.9±0.2, and 21.5±0.2.
561. Compound I Fumaric Acid (Salt or Co-Crystal) Form A according to Embodiment 552, characterized by an X-ray powder diffractogram comprising signals at 14.4±0.2 two-theta, 14.6±0.2 two-theta, 16.9±0.2 two-theta, 20.7±0.2 two-theta, 20.9±0.2 two-theta, and 21.5±0.2 two-theta.
562. Compound I Fumaric Acid (Salt or Co-Crystal) Form A according to Embodiment 552, characterized by an X-ray powder diffractogram comprising (a) a signal at 21.5±0.2 two-theta and/or a signal at 16.9±0.2 two-theta; and (b) a signal at one, two, three, four, five, six, seven, eight, nine, ten, or more two-theta values chosen from 9.5±0.2, 14.4±0.2, 14.6±0.2, 15.6±0.2, 16.9±0.2, 17.3±0.2, 17.5±0.2, 19.1±0.2, 19.5±0.2, 19.7±0.2, 20.7±0.2, 20.9±0.2, 21.0±0.2, 22.5±0.2, 23.2±0.2, 25.7±0.2, 28.3±0.2, and 29.4±0.2.
563. Compound I Fumaric Acid (Salt or Co-Crystal) Form A according to Embodiment 552, characterized by an X-ray powder diffractogram substantially similar to that in FIG. 45.
564. Compound I Fumaric Acid (Salt or Co-Crystal) Form A according to any one of Embodiments 552 to 563, characterized by a TGA thermogram showing minimal weight loss from ambient temperature up to 100° C.
565. Compound I Fumaric Acid (Salt or Co-Crystal) Form A according to any one of Embodiments 552 to 563, characterized by a TGA thermogram substantially similar to that in FIG. 48.
566. Compound I Fumaric Acid (Salt or Co-Crystal) Form A according to any one of Embodiments 552 to 565, characterized by a DSC curve having two endothermic peaks at about 137° C. and 165° C.
567. Compound I Fumaric Acid (Salt or Co-Crystal) Form A according to any one of Embodiments 552 to 565, characterized by a DSC curve substantially similar to that in FIG. 49.
568. Compound I Fumaric Acid (Salt or Co-Crystal) Form A according to any one of Embodiments 552 to 567, characterized by a ¹³C NMR spectrum comprising one or more signals chosen from 172.4±0.2 ppm, 128.1±0.2 ppm, 72.9±0.2 ppm, and 17.2±0.2 ppm.
569. Compound I Fumaric Acid (Salt or Co-Crystal) Form A according to any one of Embodiments 552 to 567, characterized by a ¹³C NMR spectrum comprising two or more signals chosen from 172.4±0.2 ppm, 128.1±0.2 ppm, 72.9±0.2 ppm, and 17.2±0.2 ppm.
570. Compound I Fumaric Acid (Salt or Co-Crystal) Form A according to any one of Embodiments 552 to 567, characterized by a ¹³C NMR spectrum comprising three or more signals chosen from 172.4±0.2 ppm, 128.1±0.2 ppm, 72.9±0.2 ppm, and 17.2±0.2 ppm.
571. Compound I Fumaric Acid (Salt or Co-Crystal) Form A according to any one of Embodiments 552 to 567, characterized by a ¹³C NMR spectrum comprising signals at 172.4±0.2 ppm, 128.1±0.2 ppm, 72.9±0.2 ppm, and 17.2±0.2 ppm.
572. Compound I Fumaric Acid (Salt or Co-Crystal) Form A according to any one of Embodiments 552 to 567, characterized by a ¹³C NMR spectrum comprising one or more (e.g., two or more, three or more, four or more, etc.) signals chosen from 172.4±0.2 ppm, 171.4±0.2 ppm, 148.4±0.2 ppm, 143.8±0.2 ppm, 142.1±0.2 ppm, 135.5±0.2 ppm, 130.7±0.2 ppm, 128.1±0.2 ppm, 127.3±0.2 ppm, 124.3±0.2 ppm, 121.5±0.2 ppm, 72.9±0.2 ppm, 65.7±0.2 ppm, 61.8±0.2 ppm, 50.8±0.2 ppm, 48.3±0.2 ppm, 47.3±0.2 ppm, 42.0±0.2 ppm, 38.3±0.2 ppm, 34.6±0.2 ppm, and 17.2±0.2 ppm.
573. Compound I Fumaric Acid (Salt or Co-Crystal) Form A according to any one of Embodiments 552 to 567, characterized by a ¹³C NMR spectrum substantially similar to that in FIG. 46.
574. Compound I Fumaric Acid (Salt or Co-Crystal) Form A according to any one of Embodiments 552 to 573, characterized by a ¹⁹F MAS spectrum comprising a single signal at −55.8±0.2 ppm.
575. Compound I Fumaric Acid (Salt or Co-Crystal) Form A according to any one of Embodiments 552 to 573, characterized by a ¹⁹F MAS spectrum substantially similar to that in FIG. 47.
576. A method of preparing Compound I Fumaric Acid (Salt or Co-Crystal) Form A comprising:

adding a vial containing ceramic beads and water to a high through-put ball-mill containing a 3:4 ratio of Compound I Monohydrate and fumaric acid;

running ball mill for three cycles of 60 seconds with 10 second pauses between cycles;

place in vacuum oven at 45° C. overnight; and

isolating the solids.

577. Compound I free form Form B.
578. Compound I free form Form B according to Embodiment 577, characterized by an X-ray powder diffractogram comprising a signal at 21.6 two-theta.
579. Compound I free form Form B according to Embodiment 577, characterized by an X-ray powder diffractogram comprising a signal at 13.9 two-theta.
580. Compound I free form Form B according to Embodiment 577, characterized by an X-ray powder diffractogram comprising a signal at 19.1 two-theta.
581. Compound I free form Form B according to Embodiment 577, characterized by an X-ray powder diffractogram comprising a signal at 11.7 two-theta.
582. Compound I free form Form B according to Embodiment 577, characterized by an X-ray powder diffractogram comprising a signal at 14.2 two-theta.
583. Compound I free form Form B according to Embodiment 577, characterized by an X-ray powder diffractogram comprising a signal at 24.6 two-theta.
584. Compound I free form Form B according to Embodiment 577, characterized by an X-ray powder diffractogram comprising signals at two or more two-theta values chosen from 11.7±0.2, 13.9±0.2, 14.2±0.2, 19.1±0.2, 21.6±0.2, and 24.6±0.2.
585. Compound I free form Form B according to Embodiment 577, characterized by an X-ray powder diffractogram comprising signals at three or more two-theta values chosen from 11.7±0.2, 13.9±0.2, 14.2±0.2, 19.1±0.2, 21.6±0.2, and 24.6±0.2.
586. Compound I free form Form B according to Embodiment 577, characterized by an X-ray powder diffractogram comprising signals at four or more two-theta values chosen from 11.7±0.2, 13.9±0.2, 14.2±0.2, 19.1±0.2, 21.6±0.2, and 24.6±0.2.
587. Compound I free form Form B according to Embodiment 577, characterized by an X-ray powder diffractogram comprising signals at five or more two-theta values chosen from 11.7±0.2, 13.9±0.2, 14.2±0.2, 19.1±0.2, 21.6±0.2, and 24.6±0.2.
588. Compound I free form Form B according to Embodiment 577, characterized by an X-ray powder diffractogram comprising signals at 11.7±0.2 two-theta, 13.9±0.2 two-theta, 14.2±0.2 two-theta, 19.1±0.2 two-theta, 21.6±0.2 two-theta, and 24.6±0.2 two-theta.
589. Compound I free form Form B according to Embodiment 577, characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) a signal at one or more two-theta values chosen from 13.1±0.2, 20.6±0.2, 17.5±0.2, 15.8±0.2, and 18.9±0.2.
590. Compound I free form Form B according to Embodiment 577, characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, and 20.6±0.2 two-theta.
591. Compound I free form Form B according to Embodiment 577, characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, 20.6±0.2 two-theta, and 17.5±0.2 two-theta.
592. Compound I free form Form B according to Embodiment 577, characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, 20.6±0.2 two-theta, 17.5±0.2 two-theta, and 15.8±0.2 two-theta.
593. Compound I free form Form B according to Embodiment 577, characterized by an X-ray powder diffractogram comprising (a) a signal at one or more two-theta values chosen from 11.7±0.2, 13.9±0.2, 14.2±0.2, 19.1±0.2, 21.6±0.2, and 24.6±0.2 and (b) signals at 13.1±0.2 two-theta, 20.6±0.2 two-theta, 17.5±0.2 two-theta, 15.8±0.2 two-theta, and 18.9±0.2 two-theta.
594. Compound I free form Form B according to Embodiment 577, characterized by an X-ray powder diffractogram substantially similar to that in FIG. 50.
595. Compound I free form Form B according to any one of Embodiments 577 to 594, characterized by a TGA thermogram showing minimal weight loss from ambient temperature up to 180° C.
596. Compound I free form Form B according to any one of Embodiments 577 to 594, characterized by a TGA thermogram substantially similar to that in FIG. 53.
597. Compound I free form Form B according to any one of Embodiments 577 to 596, characterized by a DSC curve having a broad endothermic peak at about 132° C.
598. Compound I free form Form B according to any one of Embodiments 577 to 596, characterized by a DSC curve substantially similar to that in FIG. 54.
599. Compound I free form Form B according to any one of Embodiments 577 to 598, characterized by a ¹³C NMR spectrum comprising one or more signals chosen from 152.2±0.2 ppm, 148.1±0.2 ppm, and 140.0±0.2 ppm.
600. Compound I free form Form B according to any one of Embodiments 577 to 598, characterized by a ¹³C NMR spectrum comprising one or more signals chosen from 73.7±0.2 ppm, 47.9±0.2 ppm, and 23.5±0.2 ppm.
601. Compound I free form Form B according to any one of Embodiments 577 to 598, characterized by a ¹³C NMR spectrum comprising (a) one or more signals chosen from 152.2±0.2 ppm, 148.1±0.2 ppm, and 140.0±0.2 ppm and (b) one or more signals chosen from 73.7±0.2 ppm, 47.9±0.2 ppm, and 23.5±0.2 ppm.
602. Compound I free form Form B according to any one of Embodiments 577 to 598, characterized by a ¹³C NMR spectrum comprising signals at 152.2±0.2 ppm, 148.1±0.2 ppm, and 140.0±0.2 ppm.
603. Compound I free form Form B according to any one of Embodiments 577 to 598, characterized by a ¹³C NMR spectrum comprising signals at 73.7±0.2 ppm, 47.9±0.2 ppm, and 23.5±0.2 ppm.
604. Compound I free form Form B according to any one of Embodiments 577 to 598, characterized by a ¹³C NMR spectrum comprising signals at 152.2±0.2 ppm, 148.1±0.2 ppm, 140.0±0.2 ppm, 73.7±0.2 ppm, 47.9±0.2 ppm, and 23.5±0.2 ppm.
605. Compound I free form Form B according to any one of Embodiments 577 to 598, characterized by a ¹³C NMR spectrum substantially similar to that in FIG. 51.
606. Compound I free form Form B according to any one of Embodiments 577 to 605, characterized by a ¹⁹F MAS spectrum comprising a single signal at −54.8±0.2 ppm.
607. Compound I free form Form B according to any one of Embodiments 577 to 605, characterized by a ¹⁹F MAS spectrum substantially similar to that in FIG. 52.
608. Compound I free form Form B according to any one of Embodiments 577 to 605, characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 8.1 ± 0.1 Å | α | 90° |
| b | 11.8 ± 0.1 Å | β | 90° |
| c | 18.9 ± 0.1 Å | γ | 90°. |

609. Compound I free form Form B according to any one of Embodiments 577 to 605, characterized by an orthorhombic crystal system, a P2₁2₁2₁ space group, and unit cell dimensions measured at 298 K on a Bruker diffractometer equipped Cu Kα radiation (λ=1.54178 Å) of:

| | | | |
|---|---|---|---|
| a | 8.2 ± 0.1 Å | α | 90° |
| b | 11.9 ± 0.1 Å | β | 90° |
| c | 19.1 ± 0.1 Å | γ | 90°. |

610. A method of preparing Compound I free form Form B comprising:

heating Compound I Monohydrate to 120° C. for two hours;

cooling oven to 90° C. with amorphous material in the oven and maintaining at 90° C. for 5 days; and

isolating the solid Compound I free form Form B.

611. Compound I free form Form C.
612. Compound I free form Form C according to Embodiment 611, characterized by an X-ray powder diffractogram comprising a signal at 11.1 two-theta.
613. Compound I free form Form C according to Embodiment 611, characterized by an X-ray powder diffractogram comprising a signal at 25.7 two-theta.
614. Compound I free form Form C according to Embodiment 611, characterized by an X-ray powder diffractogram comprising a signal at 14.7 two-theta.
615. Compound I free form Form C according to Embodiment 611, characterized by an X-ray powder diffractogram comprising a signal at 11.7 two-theta.
616a. Compound I free form Form C according to Embodiment 611, characterized by an X-ray powder diffractogram comprising a signal at 21.09 two-theta.
616b. Compound I free form Form C according to Embodiment 611, characterized by an X-ray powder diffractogram comprising a signal at 25.9 two-theta.
617. Compound I free form Form C according to Embodiment 611, characterized by an X-ray powder diffractogram comprising signals at two or more two-theta values chosen from 11.1±0.2, 14.7±0.2, 21.0±0.2, and 25.7±0.2.
618. Compound I free form Form C according to Embodiment 611, characterized by an X-ray powder diffractogram comprising signals at three or more two-theta values chosen from 11.1±0.2, 14.7±0.2, 21.0±0.2, and 25.7±0.2.
619. Compound I free form Form C according to Embodiment 611, characterized by an X-ray powder diffractogram comprising signals at 11.1±0.2 two-theta, 14.7±0.2 two-theta, 21.0±0.2, and 25.7±0.2 two-theta.
620. Compound I free form Form C according to Embodiment 611, characterized by an X-ray powder diffractogram comprising (a) a signal at one or more two-theta values chosen from 11.1±0.2, 14.7±0.2, 21.0±0.2, and 25.7±0.2, and (b) a signal at one or more two-theta values chosen from 9.5±0.2, 12.9±0.2, 15.4±0.2, 17.7±0.2, 18.6±0.2, and 25.9±0.2.
621. Compound I free form Form C according to Embodiment 611, characterized by an X-ray powder diffractogram comprising (a) a signal at one or more two-theta values chosen from 11.1±0.2, 14.7±0.2, 21.0±0.2, and 25.7±0.2, and (b) a signal at two or more two-theta values chosen from 9.5±0.2, 12.9±0.2, 15.4±0.2, 17.7±0.2, 18.6±0.2, and 25.9±0.2.
622. Compound I free form Form C according to Embodiment 611, characterized by an X-ray powder diffractogram comprising (a) a signal at one or more two-theta values chosen from 11.1±0.2, 14.7±0.2, 21.0±0.2, and 25.7±0.2, and (b) a signal at three or more two-theta values chosen from 9.5±0.2, 12.9±0.2, 15.4±0.2, 17.7±0.2, 18.6±0.2, and 25.9±0.2.
623. Compound I free form Form C according to Embodiment 611, characterized by an X-ray powder diffractogram comprising (a) a signal at one or more two-theta values chosen from 11.1±0.2, 14.7±0.2, 21.0±0.2, and 25.7±0.2, and (b) a signal at four or more two-theta values chosen from 9.5±0.2, 12.9±0.2, 15.4±0.2, 17.7±0.2, 18.6±0.2, and 25.9±0.2.
624. Compound I free form Form C according to Embodiment 611, characterized by an X-ray powder diffractogram comprising (a) a signal at two or more two-theta values chosen from 11.1±0.2, 14.7±0.2, 21.0±0.2, and 25.7±0.2, and (b) a signal at one or more two-theta values chosen from 9.5±0.2, 12.9±0.2, 15.4±0.2, 17.7±0.2, 18.6±0.2, and 25.9±0.2.
625. Compound I free form Form C according to Embodiment 611, characterized by an X-ray powder diffractogram substantially similar to that in FIG. 55.
626. Compound I free form Form C according to any one of Embodiments 611 to 625, characterized by a TGA thermogram showing minimal weight loss from ambient temperature up to 190° C.
627. Compound I free form Form C according to any one of Embodiments 611 to 625, characterized by a TGA thermogram substantially similar to that in FIG. 58.
628. Compound I free form Form C according to any one of Embodiments 611 to 627, characterized by a DSC curve having an endothermic peak at about 134° C.
629. Compound I free form Form C according to any one of Embodiments 611 to 627, characterized by a DSC curve substantially similar to that in FIG. 59.
630. Compound I free form Form C according to any one of Embodiments 611 to 629, characterized by a ¹³C NMR spectrum comprising one or more signals chosen from 149.6±0.2 ppm, 149.2±0.2 ppm, and 137.1±0.2 ppm.
631. Compound I free form Form C according to any one of Embodiments 611 to 629, characterized by a ¹³C NMR spectrum comprising one or more signals chosen from 74.7±0.2 ppm, 62.4±0.2 ppm, 48.3±0.2 ppm, and 24.6±0.2 ppm.
632. Compound I free form Form C according to any one of Embodiments 611 to 629, characterized by a ¹³C NMR spectrum comprising (a) one or more signals chosen from 149.6±0.2 ppm, 149.2±0.2 ppm, and 137.1±0.2 ppm; and (b) one or more signals chosen from 74.5±0.2 ppm, 62.4±0.2 ppm, 48.3±0.2 ppm, and 24.6±0.2 ppm.
633. Compound I free form Form C according to any one of Embodiments 611 to 629, characterized by a ¹³C NMR spectrum comprising signals at 149.6±0.2 ppm, 149.2±0.2 ppm, and 137.1±0.2 ppm.
634. Compound I free form Form C according to any one of Embodiments 611 to 629, characterized by a ¹³C NMR spectrum comprising signals at 74.5±0.2 ppm, 62.4±0.2 ppm, 48.3±0.2 ppm, and 24.6±0.2 ppm.
635. Compound I free form Form C according to any one of Embodiments 611 to 629, characterized by a ¹³C NMR spectrum comprising signals at 149.6±0.2 ppm, 149.2±0.2 ppm, 137.1±0.2 ppm, 74.5±0.2 ppm, 62.4±0.2 ppm, 48.3±0.2 ppm, and 24.6±0.2 ppm.
636. Compound I free form Form C according to any one of Embodiments 611 to 629, characterized by a ¹³C NMR spectrum substantially similar to that in FIG. 56.
637. Compound I free form Form C according to any one of Embodiments 611 to 636, characterized by a ¹⁹F MAS spectrum comprising a single signal at −54.0±0.2 ppm.
638. Compound I free form Form C according to any one of Embodiments 611 to 636, characterized by a ¹⁹F MAS spectrum substantially similar to that in FIG. 57.
639. Compound I free form free form Form C according to any one of Embodiments 611 to 638, characterized by an orthorhombic crystal system, a P2₁2₁2₁ space group, and unit cell dimensions measured at 100 K on a Bruker diffractometer equipped Cu Kα radiation (λ=1.54178 Å) of:

| | | | |
|---|---|---|---|
| a | 10.1 ± 0.1 Å | α | 90° |
| b | 12.5 ± 0.1 Å | β | 90° |
| c | 13.4 ± 0.1 Å | γ | 90°. |

640. A method of preparing Compound I free form Form C comprising:

obtaining a seed of Compound I free form Form C by thermal treatment on a physical mixture Compound I monohydrate and Compound II free form Form C in a TGA pan;

thermal treating with TGA ramping at 10° C. per min to 120° C., isothermal at 120° C. for 60 minutes, and then cooling at 2° C. per min down to 25° C.;

adding the seed produced with this thermal treatment into a Compound I monohydrate heptane slurry and maintaining at 50° C. for 7 days; and

isolating the solid Compound I free form Form C. 641. Compound I Phosphate Salt Form B. 642. Compound I Phosphate Salt Form B according to Embodiment 641, characterized by an X-ray powder diffractogram comprising a signal at 9.7±0.2 two-theta.

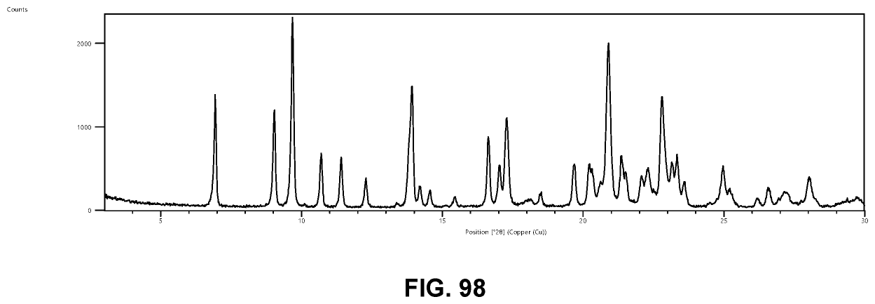

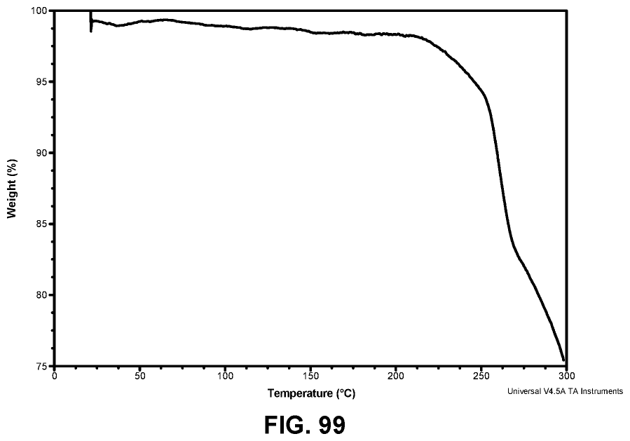

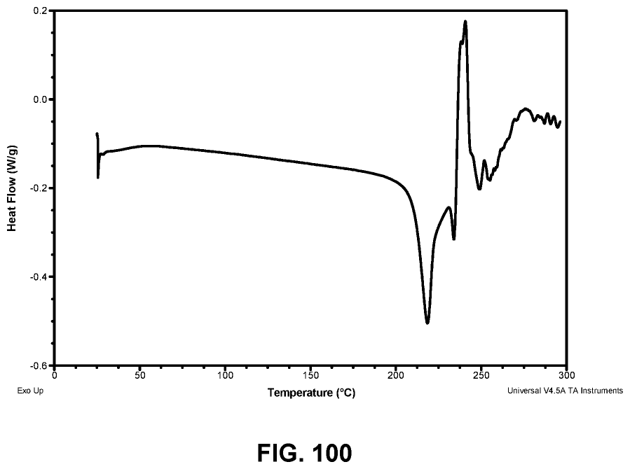

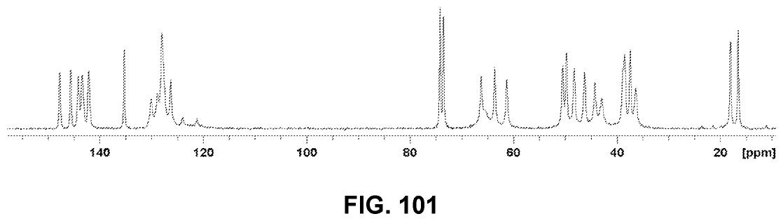

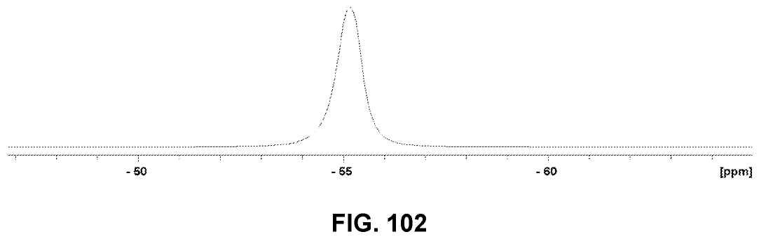

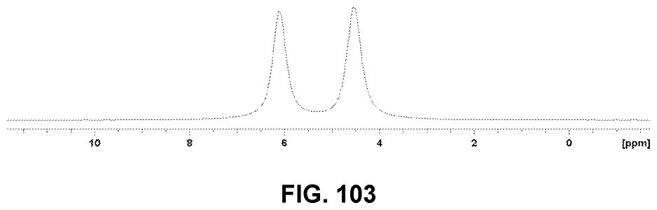

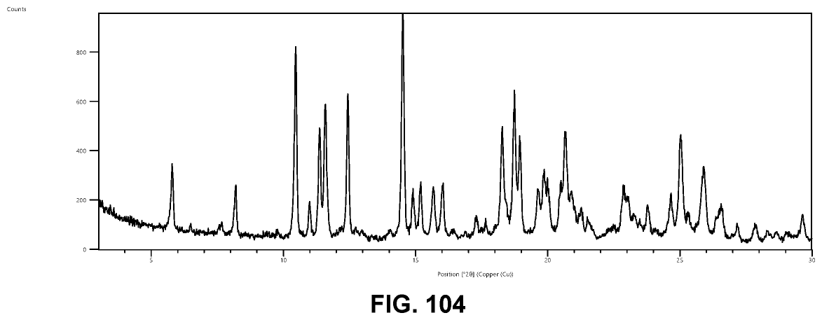

643. Compound I Phosphate Salt Form B according to Embodiment 641, characterized by an X-ray powder diffractogram comprising a signal at 13.9±0.2 two-theta.
644. Compound I Phosphate Salt Form B according to Embodiment 641, characterized by an X-ray powder diffractogram comprising a signal at 17.3±0.2 two-theta.
645. Compound I Phosphate Salt Form B according to Embodiment 641, characterized by an X-ray powder diffractogram comprising signals at two or more two-theta values chosen from 9.7±0.2, 13.9±0.2, and 17.3±0.2.
646. Compound I Phosphate Salt Form B according to Embodiment 641, characterized by an X-ray powder diffractogram comprising signals at 9.7±0.2 two-theta, 13.9±0.2 two-theta, and 17.3±0.2 two-theta.
647. Compound I Phosphate Salt Form B according to Embodiment 641, characterized by an X-ray powder diffractogram comprising (a) a signal at one or more two-theta values chosen from 9.7±0.2, 13.9±0.2, and 17.3±0.2; and (b) a signal at one or more two-theta values chosen from 6.9±0.2, 16.6±0.2, 17.0±0.2, 20.9±0.2, and 22.8±0.2.
648. Compound I Phosphate Salt Form B according to Embodiment 641, characterized by an X-ray powder diffractogram comprising (a) a signal at one or more two-theta values chosen from 9.7±0.2, 13.9±0.2, and 17.3±0.2; and (b) a signal at two or more two-theta values chosen from 6.9±0.2, 16.6±0.2, 17.0±0.2, 20.9±0.2, and 22.8±0.2.
649. Compound I Phosphate Salt Form B according to Embodiment 641, characterized by an X-ray powder diffractogram comprising (a) a signal at one or more two-theta values chosen from 9.7±0.2, 13.9±0.2, and 17.3±0.2; and (b) a signal at three or more two-theta values chosen from 6.9±0.2, 16.6±0.2, 17.0±0.2, 20.9±0.2, and 22.8±0.2.
650. Compound I Phosphate Salt Form B according to Embodiment 641, characterized by an X-ray powder diffractogram comprising (a) a signal at one or more two-theta values chosen from 9.7±0.2, 13.9±0.2, and 17.3±0.2; and (b) a signal at four or more two-theta values chosen from 6.9±0.2, 16.6±0.2, 17.0±0.2, 20.9±0.2, and 22.8±0.2.
651a. Compound I Phosphate Salt Form B according to Embodiment 641, characterized by an X-ray powder diffractogram comprising (a) a signal at two or more two-theta values chosen from 99.7±0.2, 13.9±0.2, and 17.3±0.2; and (b) a signal at one or more two-theta values chosen from 6.9±0.2, 16.6±0.2, 17.0±0.2, 20.9±0.2, and 22.8±0.2.
651b. Compound I Phosphate Salt Form B according to Embodiment 641, characterized by an X-ray powder diffractogram comprising signal a at two-theta values of 9.7±0.2, 13.9±0.2, 17.3±0.2, 6.9±0.2, 16.6±0.2, 17.0±0.2, 20.9±0.2, and 22.8±0.2.
652. Compound I Phosphate Salt Form B according to Embodiment 641, characterized by an X-ray powder diffractogram substantially similar to that in FIG. 98.
653. Compound I Phosphate Salt Form B according to any one of Embodiments 641 to 652, characterized by a TGA thermogram showing minimal weight loss from ambient temperature up to 210° C.
654. Compound I Phosphate Salt Form B according to any one of Embodiments 641 to 652, characterized by a TGA thermogram substantially similar to that in FIG. 99.
655. Compound I Phosphate Salt Form B according to any one of Embodiments 641 to 654, characterized by a DSC curve having endothermic peaks at about 218° C. and at about 235° C.
656. Compound I Phosphate Salt Form B according to any one of Embodiments 641 to 654, characterized by a DSC curve substantially similar to that in FIG. 100.
657. Compound I Phosphate Salt Form B according to any one of Embodiments 641 to 656, characterized by a ¹³C NMR spectrum comprising a signal at 48.2±0.2 ppm or 37.4±0.2 ppm.
658. Compound I Phosphate Salt Form B according to any one of Embodiments 641 to 656, characterized by a ¹³C NMR spectrum comprising signals at 48.2±0.2 ppm and 37.4±0.2 ppm.
659. Compound I Phosphate Salt Form B according to any one of Embodiments 641 to 656, characterized by a ¹³C NMR spectrum comprising (a) a signals at 48.2±0.2 ppm or 37.4±0.2 ppm; and (b) one or more signals chosen from 128.0±0.2 ppm, 74.2±0.2 ppm, and 66.2±0.2 ppm.
660. Compound I Phosphate Salt Form B according to any one of Embodiments 641 to 656, characterized by a ¹³C NMR spectrum comprising (a) signals at 48.2±0.2 ppm and 37.4±0.2 ppm; and (b) one or more signals chosen from 128.0±0.2 ppm, 74.2±0.2 ppm, and 66.2±0.2 ppm.
661. Compound I Phosphate Salt Form B according to any one of Embodiments 641 to 656, characterized by a ¹³C NMR spectrum comprising (a) signals at 48.2±0.2 ppm and 37.4±0.2 ppm; and (b) two or more signals chosen from 128.0±0.2 ppm, 74.2±0.2 ppm, and 66.2±0.2 ppm.
662. Compound I Phosphate Salt Form B according to any one of Embodiments 641 to 656, characterized by a ¹³C NMR spectrum comprising signals at 48.2±0.2 ppm, 37.4±0.2 ppm, 128.0±0.2 ppm, 74.2±0.2 ppm, and 66.2±0.2 ppm.
663. Compound I Phosphate Salt Form B according to any one of Embodiments 641 to 656, characterized by a ¹³C NMR spectrum substantially similar to that in FIG. 101.
664. Compound I Phosphate Salt Form B according to any one of Embodiments 641 to 656, characterized by a ¹⁹F MAS spectrum comprising a single signal at −55.2±0.2 ppm.
665. Compound I Phosphate Salt Form B according to any one of Embodiments 641 to 656, characterized by a ¹⁹F MAS spectrum substantially similar to that in FIG. 102.
666. Compound I Phosphate Salt Form B according to any one of Embodiments 641 to 656, characterized by a ³¹P CPMAS spectrum comprising a signal at 6.1±0.2 ppm or 4.5±0.2 ppm.
667. Compound I Phosphate Salt Form B according to any one of Embodiments 641 to 656, characterized by a ³¹P CPMAS spectrum comprising signals at 6.1±0.2 ppm and 4.5±0.2 ppm.
668. Compound I Phosphate Salt Form B according to any one of Embodiments 641 to 656, characterized by a ³¹P CPMAS spectrum substantially similar to that in FIG. 103.
669. A method of preparing Compound I Phosphate Salt Form B comprising:

adding 1-pentanol to Compound I Phosphate Salt Hydrate Form A;

stirring at ambient temperature for 2 weeks;

centrifuging and vacuum drying at 40° C. for 7 days; and

isolating the solid Compound I Phosphate Salt Form B.

670. Compound I Phosphate Salt Form C.

671. Compound I Phosphate Salt Form C according to Embodiment 670, characterized by an X-ray powder diffractogram comprising a signal at 5.8±0.2 two-theta.

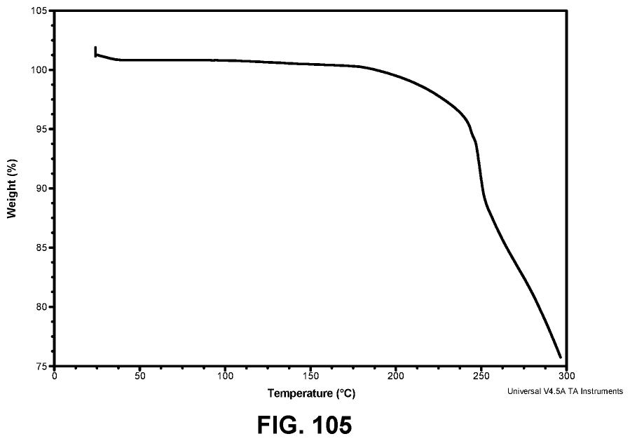

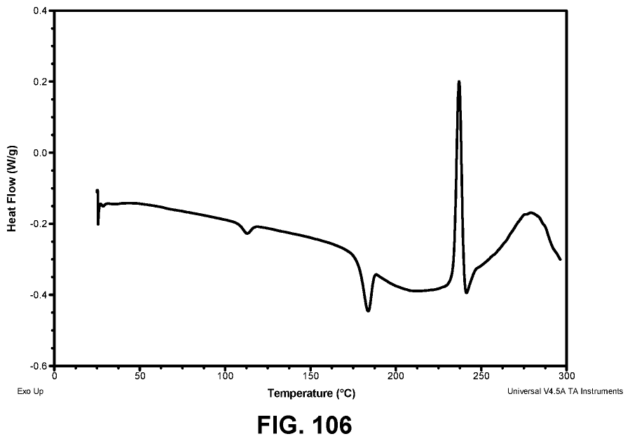

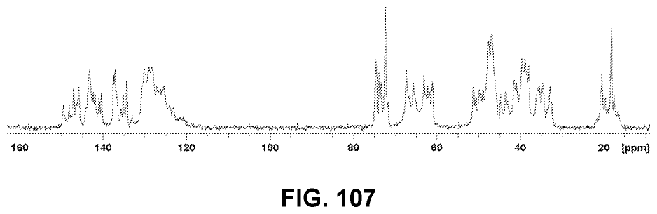

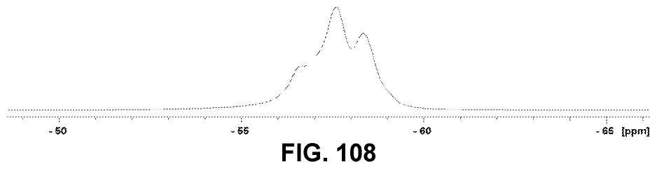

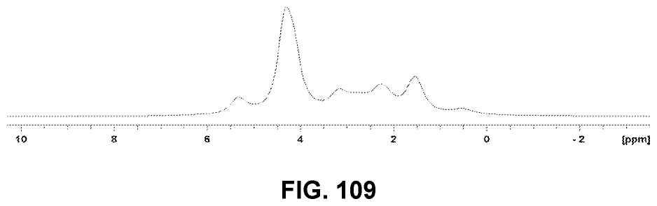

672. Compound I Phosphate Salt Form C according to Embodiment 670, characterized by an X-ray powder diffractogram comprising (a) a signal at 5.8±0.2 two-theta; and (b) one or more signals selected from 8.2±0.2, 10.4±0.2, and 14.5±0.2.
673. Compound I Phosphate Salt Form C according to Embodiment 670, characterized by an X-ray powder diffractogram comprising (a) a signal at 5.8±0.2 two-theta; and (b) two or more signals selected from 8.2±0.2, 10.4±0.2, and 14.5±0.2.
674. Compound I Phosphate Salt Form C according to Embodiment 670, characterized by an X-ray powder diffractogram comprising signals at 5.8±0.2 two-theta, 8.2±0.2 two-theta, 10.4±0.2 two-theta, and 14.5±0.2 two-theta.
675. Compound I Phosphate Salt Form C according to Embodiment 670, characterized by an X-ray powder diffractogram comprising (a) signals at 5.9±0.2 two-theta, 8.2±0.2 two-theta, 10.4±0.2 two-theta, and 14.5±0.2 two-theta; and (b) a signal at one or more two-theta values chosen from 12.4±0.2, 18.8±0.2, 11.6±0.2, and 25.0±0.2.
676. Compound I Phosphate Salt Form C according to Embodiment 670, characterized by an X-ray powder diffractogram comprising (a) signals at 5.8±0.2 two-theta, 8.2±0.2 two-theta, 10.4±0.2 two-theta, and 14.5±0.2 two-theta; and (b) a signal at two or more two-theta values chosen from 12.4±0.2, 18.8±0.2, 11.6±0.2, and 25.0±0.2.
677. Compound I Phosphate Salt Form C according to Embodiment 670, characterized by an X-ray powder diffractogram comprising (a) signals at 5.8±0.2 two-theta, 8.2±0.2 two-theta, 10.4±0.2 two-theta, and 14.5±0.2 two-theta; and (b) a signal at three or more two-theta values chosen from 12.4±0.2, 18.8±0.2, 11.6±0.2, and 25.0±0.2.
679. Compound I Phosphate Salt Form C according to Embodiment 670, characterized by an X-ray powder diffractogram substantially similar to that in FIG. 104.
680. Compound I Phosphate Salt Form C according to any one of Embodiments 670 to 679, characterized by a TGA thermogram showing minimal weight loss from ambient temperature up to 200° C.
681. Compound I Phosphate Salt Form C according to any one of Embodiments 670 to 680, characterized by a TGA thermogram substantially similar to that in FIG. 105.
682. Compound I Phosphate Salt Form C according to any one of Embodiments 670 to 681, characterized by a DSC curve having endothermic peaks at about 113° C. and at about 184° C.
683. Compound I Phosphate Salt Form C according to any one of Embodiments 670 to 682, characterized by a DSC curve substantially similar to that in FIG. 106.
684. Compound I Phosphate Salt Form C according to any one of Embodiments 670 to 683, characterized by a ¹³C NMR spectrum comprising one or more signals chosen from 39.7±0.2 ppm, 46.8±0.2 ppm, and 72.3±0.2 ppm.
685. Compound I Phosphate Salt Form C according to any one of Embodiments 670 to 683, characterized by a ¹³C NMR spectrum comprising signals at 39.7±0.2 ppm, 46.8±0.2 ppm, and 72.3±0.2 ppm.
686. Compound I Phosphate Salt Form C according to any one of Embodiments 670 to 683, characterized by a ¹³C NMR spectrum comprising (a) one or more signals chosen from 39.7±0.2 ppm, 46.8±0.2 ppm, and 72.3±0.2 ppm; and (b) one or more signals chosen from 67.3±0.2 ppm, 74.6±0.2 ppm, 137.1±0.2 ppm, and 143.2±0.2 ppm.
687. Compound I Phosphate Salt Form C according to any one of Embodiments 670 to 683, characterized by a ¹³C NMR spectrum comprising (a) one or more signals chosen from 39.7±0.2 ppm, 46.8±0.2 ppm, and 72.3±0.2 ppm; and (b) two or more signals chosen from 67.3±0.2 ppm, 74.6±0.2 ppm, 137.1±0.2 ppm, and 143.2±0.2 ppm.
688. Compound I Phosphate Salt Form C according to any one of Embodiments 670 to 683, characterized by a ¹³C NMR spectrum comprising (a) one or more signals chosen from 39.7±0.2 ppm, 46.8±0.2 ppm, and 72.3±0.2 ppm; and (b) three or more signals chosen from 67.3±0.2 ppm, 74.6±0.2 ppm, 137.1±0.2 ppm, and 143.2±0.2 ppm.
689. Compound I Phosphate Salt Form C according to any one of Embodiments 670 to 683, characterized by a ¹³C NMR spectrum comprising (a) signals at 39.7±0.2 ppm, 46.8±0.2 ppm, and 72.3±0.2 ppm; and (b) one or more signals chosen from 67.3±0.2 ppm, 74.6±0.2 ppm, 137.1±0.2 ppm, and 143.2±0.2 ppm.
690. Compound I Phosphate Salt Form C according to any one of Embodiments 670 to 683, characterized by a ¹³C NMR spectrum substantially similar to that in FIG. 107.
691. Compound I Phosphate Salt Form C according to any one of Embodiments 670 to 683, characterized by a ¹⁹F MAS spectrum comprising one or more signals chosen from −56.6±0.2 ppm, −57.6±0.2 ppm, −58.3±0.2 ppm, and −59.0±0.2 ppm.
692. Compound I Phosphate Salt Form C according to any one of Embodiments 670 to 683, characterized by a ¹⁹F MAS spectrum comprising signals at −56.6±0.2 ppm, −57.6±0.2 ppm, −58.3±0.2 ppm, and −59.0±0.2 ppm.
693. Compound I Phosphate Salt Form C according to any one of Embodiments 670 to 683, characterized by a ¹⁹F MAS spectrum substantially similar to that in FIG. 108.
694. Compound I Phosphate Salt Form C according to any one of Embodiments 670 to 683, characterized by a ³¹P CPMAS spectrum comprising one or more signals chosen from 5.3±0.2 ppm, 4.3±0.2 ppm, 3.2±0.2 ppm, 2.3±0.2 ppm, 1.5±0.2 ppm, and 0.6±0.2 ppm.
695. Compound I Phosphate Salt Form C according to any one of Embodiments 670 to 683, characterized by a ³¹P CPMAS spectrum comprising signals at 5.3±0.2 ppm, 4.3±0.2 ppm, 3.2±0.2 ppm, 2.3±0.2 ppm, 1.5±0.2 ppm, and 0.6±0.2 ppm.
696. Compound I Phosphate Salt Form C according to any one of Embodiments 670 to 683, characterized by a ³¹P CPMAS spectrum substantially similar to that in FIG. 109.
697. A method of preparing Compound I Phosphate Salt Form C comprising:

adding 1,4-dioxane to Compound I Phosphate Salt Hydrate Form A;

stirring at ambient temperature for 2 weeks;

centrifuging and vacuum drying at 40° C. for 7 days; and

isolating the solid Compound I Phosphate Salt Form C.

### 700. Compound I Phosphate Salt Crystalline Form Mixture.

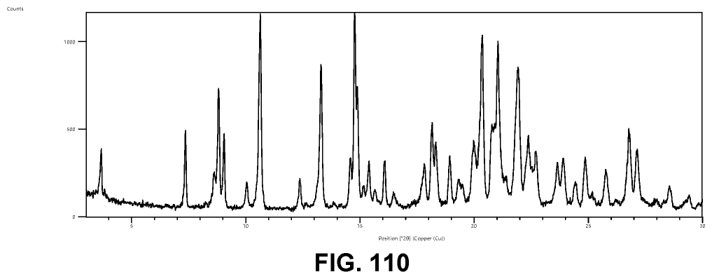

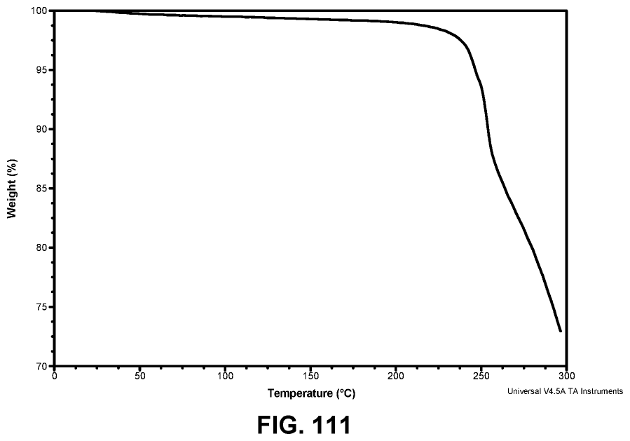

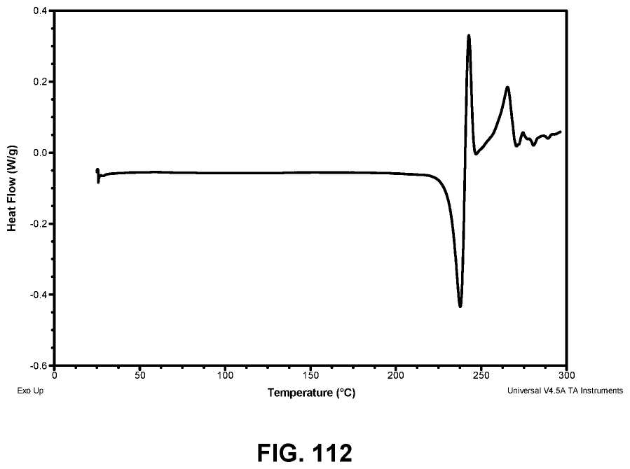

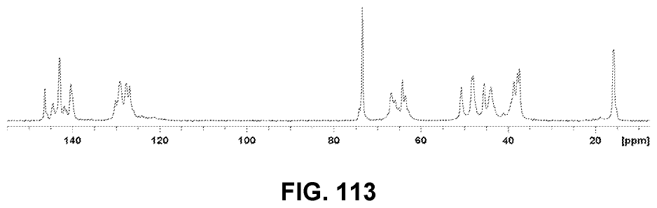

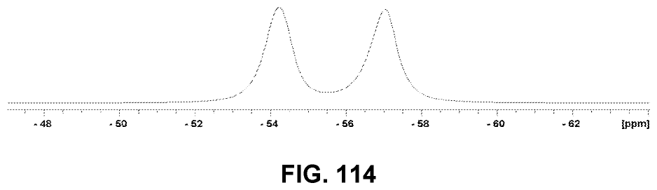

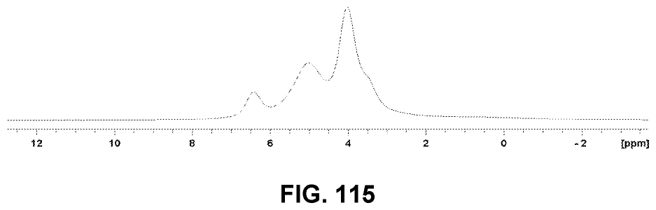

701. Compound I Phosphate Salt Crystalline Form Mixture according to Embodiment 700, characterized by an X-ray powder diffractogram comprising a signal at 13.3 two-theta.
702. Compound I Phosphate Salt Crystalline Form Mixture according to Embodiment 700, characterized by an X-ray powder diffractogram comprising a signal at 27.1 two-theta.
703. Compound I Phosphate Salt Crystalline Form Mixture according to Embodiment 700, characterized by an X-ray powder diffractogram comprising signals at 13.3±0.2 two-theta and 27.1±0.2 two-theta.
704. Compound I Phosphate Salt Crystalline Form Mixture according to Embodiment 700, characterized by an X-ray powder diffractogram comprising (a) a signal at one or more two-theta values chosen from 13.3±0.2 and 27.1±0.2; and (b) a signal at one or more two-theta values chosen from 7.3±0.2, 10.6±0.2, 14.8±0.2, 20.3±0.2, 21.0±0.2 and 21.9±0.2.
705. Compound I Phosphate Salt Crystalline Form Mixture according to Embodiment 700, characterized by an X-ray powder diffractogram comprising (a) a signal at one or more two-theta values chosen from 13.3±0.2 and 27.1±0.2; and (b) a signal at two or more two-theta values chosen from 7.3±0.2, 10.6±0.2, 14.8±0.2, 20.3±0.2, 21.0±0.2 and 21.9±0.2.
706. Compound I Phosphate Salt Crystalline Form Mixture according to Embodiment 700, characterized by an X-ray powder diffractogram comprising (a) a signal at one or more two-theta values chosen from 13.3±0.2 and 27.1±0.2; and (b) a signal at three or more two-theta values chosen from 7.3±0.2, 10.6±0.2, 14.8±0.2, 20.3±0.2, 21.0±0.2 and 21.9±0.2.
707. Compound I Phosphate Salt Crystalline Form Mixture according to Embodiment 700, characterized by an X-ray powder diffractogram comprising (a) a signal at one or more two-theta values chosen from 13.3±0.2 and 27.1±0.2; and (b) a signal at four or more two-theta values chosen from 7.3±0.2, 10.6±0.2, 14.8±0.2, 20.3±0.2, 21.0±0.2, and 21.9±0.2.
708. Compound I Phosphate Salt Crystalline Form Mixture according to Embodiment 700, characterized by an X-ray powder diffractogram comprising signals at two-theta values of 7.3±0.2, 10.6±0.2, 13.3±0.2, 14.8±0.2, 20.3±0.2, and 27.1±0.2.
709. Compound I Phosphate Salt Crystalline Form Mixture according to Embodiment 700, characterized by an X-ray powder diffractogram substantially similar to that in FIG. 110.
710. Compound I Phosphate Salt Crystalline Form Mixture according to any one of Embodiments 700 to 709, characterized by a TGA thermogram showing minimal weight loss from ambient temperature up to 200° C.
711. Compound I Phosphate Salt Crystalline Form Mixture according to any one of Embodiments 700 to 709, characterized by a TGA thermogram substantially similar to that in FIG. 111.
712. Compound I Phosphate Salt Crystalline Form Mixture according to any one of Embodiments 700 to 711, characterized by a DSC curve having an endothermic peak at about 237° C.
713. Compound I Phosphate Salt Crystalline Form Mixture according to any one of Embodiments 700 to 711, characterized by a DSC curve substantially similar to that in FIG. 112.
714. Compound I Phosphate Salt Crystalline Form Mixture according to any one of Embodiments 700 to 713, characterized by a ¹³C NMR spectrum comprising one or more signals chosen from 15.7±0.2 ppm, 15.8±0.2 ppm, 45.4±0.2 ppm, 64.2±0.2 ppm, 126.8±0.2 ppm and 127.6±0.2 ppm.
715. Compound I Phosphate Salt Crystalline Form Mixture according to any one of Embodiments 700 to 713, characterized by a ¹³C NMR spectrum comprising two or more signals chosen from 15.7±0.2 ppm, 15.8±0.2 ppm, 45.4±0.2 ppm, 64.2±0.2 ppm, 126.8±0.2 ppm and 127.6±0.2 ppm.
716. Compound I Phosphate Salt Crystalline Form Mixture according to any one of Embodiments 700 to 713, characterized by a ¹³C NMR spectrum comprising three or more signals chosen from 15.7±0.2 ppm, 15.8±0.2 ppm, 45.4±0.2 ppm, 64.2±0.2 ppm, 126.8±0.2 ppm and 127.6±0.2 ppm.
717. Compound I Phosphate Salt Crystalline Form Mixture according to any one of Embodiments 700 to 713, characterized by a ¹³C NMR spectrum comprising four or more signals chosen from 15.7±0.2 ppm, 15.8±0.2 ppm, 45.4±0.2 ppm, 64.2±0.2 ppm, 126.8±0.2 ppm and 127.6±0.2 ppm.
718. Compound I Phosphate Salt Crystalline Form Mixture according to any one of Embodiments 700 to 713, characterized by a ¹³C NMR spectrum comprising signals at 15.7±0.2 ppm, 15.8±0.2 ppm, 45.4±0.2 ppm, 64.2±0.2 ppm, 126.8±0.2 ppm and 127.6±0.2 ppm.
719. Compound I Phosphate Salt Crystalline Form Mixture according to any one of Embodiments 700 to 714, characterized by a ¹³C NMR spectrum substantially similar to that in FIG. 113.
720. Compound I Phosphate Salt Crystalline Form Mixture according to any one of Embodiments 700 to 715, characterized by a ¹⁹F MAS spectrum comprising one or more signals chosen from −54.2±0.2 and −57.0±0.2 ppm.
721. Compound I Phosphate Salt Crystalline Form Mixture according to any one of Embodiments 700 to 715, characterized by a ¹⁹F MAS spectrum comprising signals at −54.2±0.2 ppm and −57.0±0.2 ppm.
722. Compound I Phosphate Salt Crystalline Form Mixture according to any one of Embodiments 700 to 715, characterized by a ¹⁹F MAS spectrum substantially similar to that in FIG. 114.
723. Compound I Phosphate Salt Crystalline Form Mixture according to any one of Embodiments 700 to 718, characterized by a ³¹P CPMAS spectrum comprising one or more signals chosen from 6.4±0.2 ppm, 5.0±0.2 ppm, 4.0±0.2 ppm, and 3.5±0.2 ppm.
724. Compound I Phosphate Salt Crystalline Form Mixture according to any one of Embodiments 700 to 718, characterized by a ³¹P CPMAS spectrum comprising signals at 6.4±0.2 ppm, 5.0±0.2 ppm, 4.0±0.2 ppm, and 3.5±0.2 ppm.
725. Compound I Phosphate Salt Crystalline Form Mixture according to any one of Embodiments 700 to 718, characterized by a ³¹P CPMAS spectrum substantially similar to that in FIG. 115.
726. A method of preparing Compound I Phosphate Salt Crystalline Form Mixture comprising:

adding 2-MeTHF to Compound I free form Monohydrate;

stirring the solution while heating from ambient temperature to 30° C.;

adding a solution of phosphoric acid and 2-MeTHF over 2 hours;

cooling the slurry to ambient temperature over 2 hours;

vacuum drying at ambient temperature overnight;

washing the wet cake with 2-MeTHF under a nitrogen bleed at 50° C.; and

isolating the solid Compound I Phosphate Salt Crystalline Form Mixture.

### EXAMPLES

In order that the disclosure described herein may be more fully understood, the following examples are set forth. It should be understood that these examples are for illustrative purposes only and are not to be construed as limiting this disclosure in any manner.

Methods of preparation, along with the structure of and physicochemical data, for Compound I and Compound II are reported in International Application No. PCT/US2021/047754, filed on Aug. 26, 2021, the contents of which are incorporated herein by reference.

The compounds of the disclosure may be made according to standard chemical practices or as described herein. Throughout the following synthetic schemes and in the descriptions for preparing compounds, the following abbreviations are used:

### Abbreviations

ACN or MeCN=Acetonitrile
AcOH=Acetic acid
AIBN=Azobisisobutyronitrile
ARP=assay ready plate
BBBPY=4,4′-Di-tert-butyl-2,2′-dipyridyl
CBzCl=Benzyl chloroformate
CDI=Carbonyldiimidazole
CDMT=2-Chloro-4,6-dimethoxy-1,3,5-triazine
CMOS=complementary metal-oxide semiconductor
CPAD=charge-integrating pixel array
CPMAS=cross polarization magic angle spinning
CPME=Cyclopentyl methyl ether
DCC=N,N′-dicyclohexylcarbodiimide
DCM=Dichloromethane
DIPEA=N,N-Diisopropylethylamine or N-ethyl-N-isopropyl-propan-2-amine
DMAP=dimethylamino pyridine
DMA or DMAc=dimethyl acetamide
DME=dimethoxyethane
DMEM=Dulbecco's modified Eagle's medium
DMF=dimethylformamide
DMSO=dimethyl sulfoxide
DPPA=diphenylphosphoryl azide
DSC=differential scanning calorimetry
EDCL=1-(3-dimethylaminopropyl)-3-ethylcarbodiimide hydrochloride
e.r.=enantiomeric ratio
EtOAc=Ethyl Acetate
EtOH=ethanol
FBS=fetal bovine serum
FLU=fluorescent values
GC=gas chromatography
HATU=[dimethylamino(triazolo[4,5-b]pyridin-3-yloxy)methylene]-dimethyl-ammonium (Phosphorus Hexafluoride Ion)
HBSS=Hank's balanced salt solution
HCl=hydrochloric acid
HDMC=N-[(5-Chloro-3-oxido-1H-benzotriazol-1-yl)-4-morpholinylmethylene]-N-methylmethanaminium hexafluorophosphate
HEPES=4-(2-hydroxyethyl)-1-piperazineethanesulfonic acid
HOBt=Hydroxybenzotriazole
HPLC=High-performance liquid chromatography
IPA=isopropyl alcohol
iPrOAc=isopropyl acetate
KMOS=K-band multi object spectrograph
LCMS=liquid chromatography mass spectrometry
LDA=lithium diisopropyl amide
LED=light emitting diode
MAS=magic angle spinning
MEK=Methyl ethyl ketone
MeOH=methanol
MFSDA=Methyl fluorosulfonyldifluoroacetate
MsOH=methanesulfonic acid
MTBE=Methyl tert-butyl ether
NaCl=sodium chloride
NaHCO₃=sodium bicarbonate
NaOH=sodium hydroxide
NIS=N-iodosuccinimide
NMM=N-methyl morpholine
NMP=N-methyl pyrrolidine
NOE=nuclear Overhauser effect
PBS=phosphate-buffered saline
Pd(dppf)₂Cl₂=[1,1′-Bis(diphenylphosphino)ferrocene]dichloropalladium(II)
PdCl₂(PPh₃)₂=Bis(triphenylphosphine)palladium(II) dichloride
PP=polypropylene
PTSA=p-Toluenesulfonic acid monohydrate
PVDF=polyvinylidene fluoride
qNMR=quantitative nuclear magnetic resonance
RH=relative humidity
RPM=revolutions per minute
SCXRD=single crystal X-ray diffraction
SFC=supercritical fluid chromatography
ssNMR=solid-state nuclear magnetic resonance
T3P=2,4,6-Tripropyl-1,3,5,2,4,6-trioxatriphosphorinane-2,4,6-trioxide
TBAB=tetrabutylammonium bromide
TBAF=tetra-n-butylammonium fluoride
TEA=triethylamine
Tet=tetracycline
TFA=trifluoroacetic acid
TFAA=trifluoroacetic anhydride
TGA=thermogravimetric analysis
THF=tetrahydrofuran
THP=tetrahydropyran
TLC=thin layer chromatography
TMSS=Tris(trimethylsilyl)silane
(R,R)-TsDPEN=(R,R)—N-(p-toluenesulfonyl)-1,2-diphenylethylenediamine
XRPD=X-ray powder diffraction

### Example 1: Synthesis of Compound I

### 1. Preparation of Compound I Synthetic Precursors

### Preparation of K2

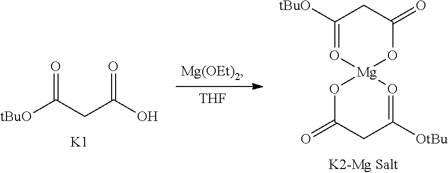

THF (3720 mL, 6.2 vol) was charged to a 5 L glass flask, then K1 (600 g, 3.47 mol, 576.92 mL, 92.6% purity by qNMR, 1 equiv) was added at 20° C. The mixture was cooled to 0° C. and Mg(OEt)₂ (198.46 g, 1.73 mol, 0.5 equiv) was charged to the reactor. The resulting mixture was stirred at 0-5° C. for 10 minutes, then warmed to 20° C. and stirred for 18 hours to give a milky white suspension. The hazy solution was distilled at 40° C. under reduced pressure to remove THF (3.1 L). n-Hexane (3.1 L) was added, and the mixture was stirred for 2 hours to give a thick slurry. The slurry was filtered, and the filter cake was washed with n-hexane (1×300 mL). The solid was dried under vacuum at 40° C. for 16 hours to provide 533.6 g of K2-Mg salt (89.8% yield). In addition to the Mg-salt, other salts of K2, such as, e.g., Na, K, and Ca, could be prepared and used in subsequent steps, e.g., in the preparation of K7.

### Preparation of K7

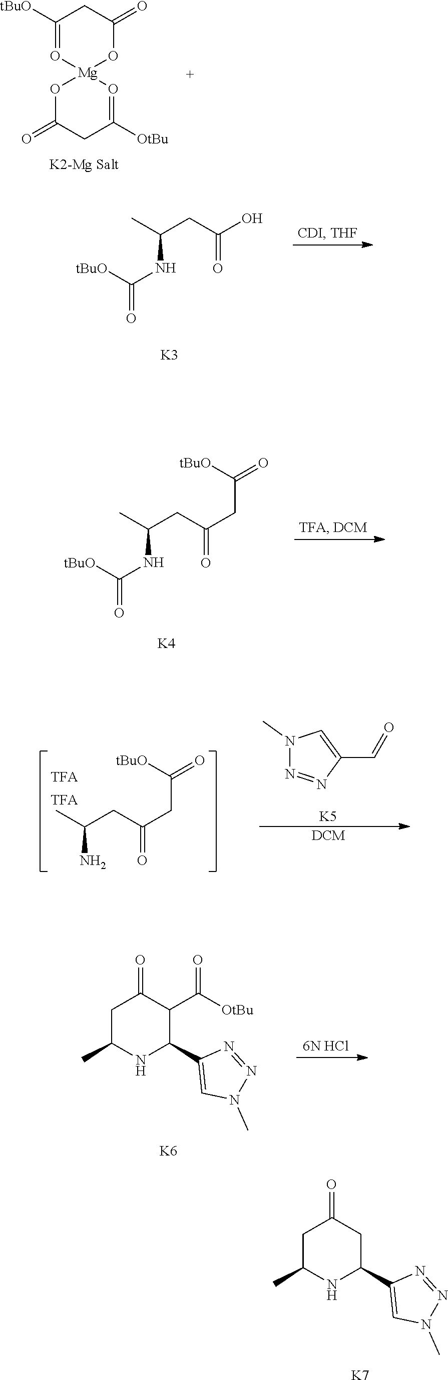

Step 1. K3 (600 g, 2.85 mol, 1 equiv, 96.5% purity by qNMR) was dissolved in anhydrous THF (3660 mL) in a 5000 mL glass flask. CDI (508.15 g, 3.13 mol, 1.1 equiv) was charged to the flask in 5 portions over 15 minutes to give a solution. Optionally, this step could be performed with other peptide coupling reagents, such as combinations of a carbodiimide (e.g., EDCl or DCC) and an appropriate activator (e.g., HOBt or DMAP); phosphonium and uronium reagents; thionyl chloride; and oxalyl chloride The resulting reaction mixture was stirred at 18° C. for 2.5 hours. K2-Mg salt (755.77 g, 2.02 mol, 91.7% purity, 0.71 eq) was charged to the reactor in 5 portions over 8 minutes. The resulting suspension was stirred for 18 hours at 18° C. The reaction mixture was diluted with methyl tert-butyl ether (1.8 L, 3 vol) and treated with 2 N HCl (7.1 L) to adjust the pH to 2.0-3.0. The organic layer was separated. The organic layer was combined and washed with saturated sodium bicarbonate (3.3 L). The organic layer was dried over anhydrous sodium sulfate and filtered, and the filtrate was evaporated at 40° C. under reduced pressure to give 862.3 g of K4 (96.9% yield).

Steps 2 and 3. A solution of K4 (570.0 g, 1.83 mol, 96.7% purity by qNMR, 1 equiv) in dichloromethane (2850 mL, 5 vol) was cooled to 5° C. At 0-5° C. and charged with trifluoroacetic acid (859.15 g, 7.54 mol, 557.89 mL, 4.12 equiv) over 80 minutes. Alternatively, this step could be accomplished with other organic acids like sulfonic acids (such as, e.g., methanesulfonic acid, benzenesulfonic acid, p-toluenesulfonic acid), phosphonic acids and carboxylic acids or mineral acids like HCl or H₃PO₄. The resulting solution was stirred at 5° C. for 1 hour, then warmed to 20° C. and stirred for 18 hours. K5 (180.76 g, 1.59 mol, 97.8% purity, 0.87 equiv) was charged as a solid in one portion, and the resulting solution was stirred for 18 hours at 20° C. The reaction mixture was diluted with saturated brine (1.14 L, 2 vol), cooled to 5 to 10° C., and then adjusted to pH 10 with 6 N sodium hydroxide (950 mL). The organic layer was separated and dried over sodium sulfate (400 g). The resulting solution was distilled at 30° C. under reduced pressure to remove DCM (1 L). MTBE (1.14 L) was charged, and the mixture was evaporated to dryness under reduced pressure to give an off-white solid 533.5 g. The residue was diluted with methyl tert-butyl ether (3.2 L, 6 vol) and stirred at 10-20° C. for 24 hours. The mixture was filtered, and the filter cake was washed with fresh methyl tert-butyl ether (453 mL, 0.85 vol) and dried under vacuum at 45° C. for 1 hour to provide 290.4 g of K6 (62.0% yield).

Step 4. To an aqueous solution of HCl (6 M, 1.52 L, 8.83 eq) was added K6 (303 g, 1.03 mol, 1 eq) in nine portions at 30-35° C. in a 3000 mL three-necked, round-bottomed flask. The mixture was stirred at 35° C. for 1 hour. A light yellow solution was obtained. TLC and LCMS analysis indicated K6 was reacted completely. HPLC indicated about 0.03% of K6 remained. When the reaction was completed, the mixture was cooled to 5° C. and charged with 3 g of solid K₃PO₄. 1.11 g of 45% KOH solution was charged portion-wise at a rate to keep the temperature less than 30° C. 156 g of 45% KOH was charged resulting in a pH of 11-12. The mixture was extracted with DCM (6×900 mL). The combined organic layers were dried over sodium sulfate (300 g) and concentrated at 25° C. under vacuum until a heavy slurry of product was obtained. n-Heptane (200 mL) was added, and the mixture was further concentrated at 25° C. to remove solvents (200 mL). The resulting solution was filtered, and the filter cake was washed with n-heptane (200 mL). The solid was dried under vacuum for 10 hours at 40° C. to give 186 g of K7 (92.9% yield).

### Preparation of K8

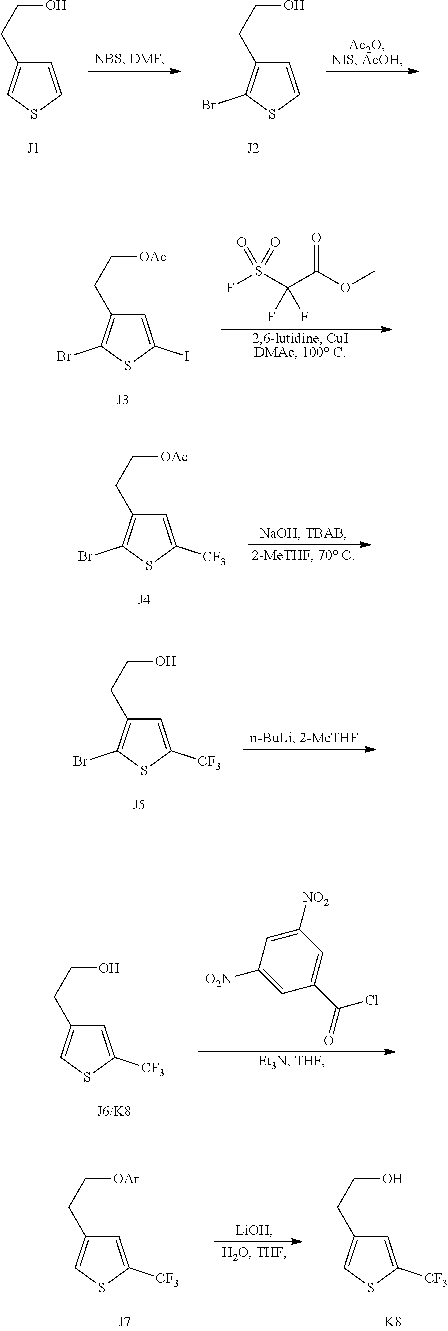

Step 1 (J2): J1 (85.0 kg, 663.1 mol, 1.0 equiv) was dissolved in DMF (162.3 kg) in a 1000 L reactor with stirring under nitrogen and then cooled to −10-0° C. N-bromosuccinimide (NBS) (122.7 kg, 689.6 mol, 1.04 eq) was dissolved in DMF (241.0 kg) in a separate, 500 L reactor with stirring under nitrogen. Optionally, other brominating agents such as, e.g., bromine, 1,3-dibromo-5,5-dimethylhydantoin, and others may be used for this step. The solution of NBS was added slowly to the 1000 L reactor over 5 hours while maintaining the temperature between −10-0° C. After the addition, the reaction mixture was held at −10-0° C. for 1-2 hours. A saturated aqueous solution of NaCl (480 kg) was added to the reaction mixture followed by EtOAc (460.7 kg), and the reaction mixture was stirred for 30 minutes. The organic layer was separated and the aqueous layer was extracted with EtOAc (230.4 kg). The organic layers were combined and washed with 0.5 N HCl (420.0 kg). After separation, a saturated solution of NaCl (300 kg) was added, and the mixture was stirred for 30 minutes. The phases were separated and the organic layer was concentrated at 40-50° C. to afford J2 (147.95 kg, 92.3% purity, 75% qNMR, 80.78% yield) as a brown liquid.

Step 2 (J3): J2 (147.95 kg, qNMR 75%, 535.8 mol, 1.0 equiv) was treated with AcOH (349.65 kg) and Ac₂O (82.05 kg, 803.7 mol, 1.5 eq) in a 1000 L reactor with stirring under nitrogen. Instead of Ac₂O, a suitable acetylating reagent like acetyl chloride may be used. The mixture was heated to 90-100° C. for 5-10 hours and until less than 0.5% J2 remained by GC. The mixture was cooled to 35-40° C., N-iodosuccinimide (NIS) (138.6 kg, 616.2 mol, 1.15 eq) was added to the 1000 L reactor, and the mixture was stirred at 35-40° C. for 6-10 hours. Alternatively, a different iodinating reagent, such as, e.g., iodine (I₂), 1,3-diiodo-5,5-dimethylhydantoin, and others, may be used for this step. When less than 0.5% of the intermediate remained, the mixture was cooled to 20-30° C. and transferred to a 3000 L reactor. A mixture of MTBE/heptane (250 kg/226.4 kg) and water (333 kg) were added. The mixture was stirred for 30 minutes and then separated. The aqueous layer was extracted with a mixture of MTBE/heptane (250 kg/226.4 kg). The organic layers were combined, and a 13% solution of aqueous NaHSO₃ (510.6 kg) was added. After stirring the mixture for 30 minutes, the layers were separated, and the organic layer was washed with 1 M NaOH (461.8 kg) and water (333 kg). The organic layer was concentrated at 40-60° C. to afford J3 (220.75 kg, 92.3% purity, 85.57% qNMR, 94% yield) as a brown liquid.

Step 3 (J4): J3 (111 kg, 85.57% qNMR, 252.2 mol, 1.0 equiv), Cupper iodide (CuI) (12.06 kg, 63.3 mol, 0.25 equiv), and 2,6-lutidine (6.78 kg, 63.3 mol, 0.25 equiv) were dissolved in DMAc (356.25 kg) in a 3000 L reactor with stirring under nitrogen and then heated to 85-100° C. Methyl fluorosulfonyldifluoroacetate (MFSDA, 194.65 kg, 1013.2 mol, 4.0 equiv) was added to the 3000 L reactor while maintaining the temperature between 85-100° C. Alternatively, other suitable trifluoromethylating reagents may be used for this step. After the reaction mixture was held at 90-95° C. for 1-4 hours, less than 5.0% J3 remained and the reaction mixture was cooled to 5-15° C. In another 3000 L reactor, water (1140 kg) and n-heptane (439.3 kg) were charged, and the mixture was cooled to 10-20° C. The reaction was quenched to this reactor at 10-20° C., and the resulting mixture was stirred for 30 minutes. The layers were filtered and then separated. The aqueous phase was extracted with n-heptane (220 kg), and the combined organics were washed with 20% NaCl (570 kg) and dried with MgSO₄ (9.5 kg, 10% w/w). The mixture was filtered and concentrated at 35-45° C. to give crude J4. This same procedure was repeated on three additional batches of J4 (109.8 kg, qNMR 85.57%)+(110.2 kg, qNMR 85.1%)+(108.15 kg, qNMR 85.1%). The four total batches of crude J4 were combined and distilled to afford J4 (246.5 kg, 89.6% purity, 87% qNMR, 67.7% yield) as a yellow liquid.

Step 4 (J5): NaOH (61.63 kg, 1540.8 mol, 2.28 equiv) was dissolved in water (493 kg) in a 3000 L reactor with stirring. J4 (246.5 kg, 87% qNMR, 676.2 mol, 1.0 equiv) and tetrabutylammonium bromide (TBAB, 12.33 kg, 38.25 mol, 0.057 eq) were charged, followed by 2-MeTHF (1059.95 kg). Optionally, metal hydroxides, such as, e.g., KOH, CsOH and LiOH, may be used in this step. The reaction mixture was heated to 65-75° C. and held at that temperature for 1-4 hours, at which time less than 1.0% J4 remained by HPLC analysis. The reaction mixture was cooled to 30° C., and the phases were separated. The organic layer was washed twice with water (739.5 kg) and dried over MgSO₄ (36.98 kg). The mixture was filtered and concentrated to dryness at 40-50° C. n-Heptane (167.6 kg) was added, and the mixture was again concentrated to remove residual water. This process was repeated one time to afford J5 (203.2 kg, 89.46% qNMR, 94.57% purity, 97.72% yield) as a yellow liquid.

Step 5 (J6/K8): J5 (203.2 kg, 89.46% qNMR, 660.8 mol, 1.0 equiv) was dissolved in THF (817.2 kg) in a 2000 L reactor with stirring under nitrogen. The solution was cooled to −50 to −30° C. and charged with n-BuLi (377.5 kg, 1387.7 mol, 2.1 equiv) while maintaining the temperature between −50 to −30° C. After the reaction mixture was held at −50 to −30° C. for 1-2 hours, less than 1.0% J5 remained. The mixture was quenched into 20% aqueous NH₄Cl (671.9 kg) at 15° C., and the resulting mixture was stirred for 30 minutes and separated. The aqueous phase was extracted with EtOAc (817 kg). The combined organic phases were washed twice with 20% aqueous NH₄Cl (671.9 kg), followed by 20% aqueous NaCl (408.6 kg) and then concentrated to dryness at 40-55° C. THF (100 kg) was added, and the mixture was concentrated to remove residual water. This process was repeated one time to afford J6/K8 (147.8 kg, 89.71% purity, 83.62% qNMR, 95.41% yield) as a yellow liquid.

Step 6 (J7): J6/K8 (147.8 kg, 83.62% qNMR, 627.0 mol, 1.0 equiv) and triethylamine (95.2 kg, 940.5 mol, 1.5 equiv) were dissolved in THF (587.0 kg) in a 3000 L reactor with stirring under nitrogen. The mixture was cooled to −10-0° C. 3,5-Dinitrobenzoyl chloride (173.5 kg, 752.4 mol, 1.2 equiv) was dissolved in THF (587.0 kg) in a separate 1000 L reactor, and the resulting solution was transferred into the 3000 L reactor at −10-5° C. After the reaction mixture was warmed to 10-20° C. and stirred for 1.5-2 hours, less than 1.0% J6/K8 remained. 8% aqueous NaHCO₃ (667.4 kg) and EtOAc (500 kg) were added to the 3000 L reactor. The mixture was stirred for 30 minutes and then separated. The organic layer was washed with an 8% aqueous solution of NaHCO₃ (667.4 kg), followed by 10% aqueous NaCl (680 kg), and then concentrated at 40-55° C. n-Heptane (168 kg) was added, and the mixture was concentrated at 40-55° C. EtOAc (300 kg) and n-heptane (420 kg) were added, and the mixture was heated to 65-75° C. with stirring for 1-2 hours. The slurry was cooled to 15-25° C., was stirred for 1-2 hours, and then was filtered. The solid was treated with a combination of EtOAc (450 kg) and EtOH (352 kg), and the resulting mixture was heated to 65-75° C. with stirring for 1-2 hours. The mixture was cooled to 5-10° C., stirred for 1-2 hours, and filtered. The filter cake was washed with EtOH (50 kg) and dried at 40-50° C. to afford J7 (206.4 kg, 99.04% purity, 83.59% yield) as a light yellow solid.

Step 7 (K8): LiOH.H₂O (66.57 kg, 1586.5 mol, 3.0 equiv) was dissolved in water (619.2 kg) in a 3000 L reactor with stirring. J7 (206.4 kg, 528.8 mol, 1.0 equiv) and THF (928.8 kg) were charged. Optionally, other metal hydroxides, such as, e.g., NaOH, KOH and CsOH may be used in this step. After stirring the mixture at 30-40° C. for 3 hours, less than 1% J7 remained. The layers were separated, and the THF layer was concentrated at 40-55° C. MTBE (1548 kg) was added, and the resulting mixture was washed twice with 8% aqueous NaHCO₃ (668.7 kg) and then washed with 20% aqueous NaCl (743 kg). The mixture was dried over MgSO₄ (20.64 kg, 10% w/w) for 1-2 hours and filtered. The organic phase was concentrated at 40-50° C.

n-Heptane (138 kg) was added, and the mixture was concentrated to remove residual MTBE. This process was repeated one time, and the resulting solution was concentrated to yield K8 (89.9 kg, 98.61% qNMR, 99.24% purity, 86.72% yield) as a light yellow, brown liquid.

### Alternative Preparation of S3/J6/K8

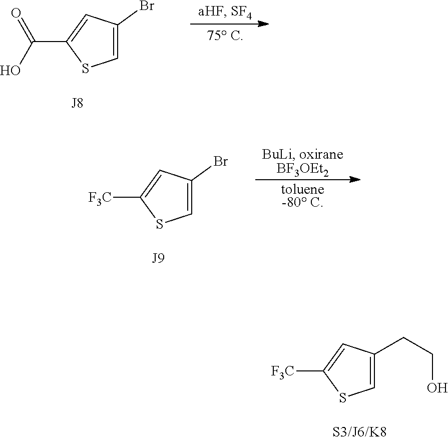

Step 1 (J9): 4-bromo-thiophene-2-carboxylic acid (Compound J8, 250 g, 1.207 mol, 1.0 equiv) was charged to a 15 L autoclave and pressure tested overnight. Anhydrous hydrofluoric acid (aHF, 250 mL, 1 vol) was cooled to −78° C. and charged to the vessel under a static vacuum over the course of 25 minutes at −32° C. SF₄ (391 g, 3.622 mol, 3.0 equiv) was charged under nitrogen over the course of 40 minutes. The reaction was heated to 75° C. for 36 h before the vessel was allowed to cool to room temperature. The volatiles were vented through a KOH scrubber and the contents of the vessel poured onto ice (500 g) using nitrogen and the reactor was rinsed with DCM (3 vol). The mixture was quenched with 3 N KOH (7.5 vol) until a pH of 13 was achieved. The layers were separated, and the aqueous layer was washed with DCM (2 vol). The combined organic layers were distilled to yield a crude residue. The crude material was purified via fractional distillation to afford 170.1 g (61%, 99.4% HPLC purity) of the desired Compound J9.

Step 2 (S3/J6/K8): A solution of Compound J9 (50 g, 0.216 mol, 1.0 equiv) in toluene (600 mL, 12 vol) was cooled to −80° C. under an N2 atmosphere. n-BuLi (2.5 M in hexanes, 93.3 mL, 0.233 mol, 1.08 equiv) was charged over the course of 60 minutes, keeping the internal reaction temperature below −78° C. After the addition was complete, the reaction mixture was stirred for 2 h at −80° C. Oxirane (37.6 g, 0.853 mol, 3.95 equiv) was purged into the reaction vessel over the course of 45 minutes, keeping the internal temperature below −75° C. The reaction was stirred for 20 minutes. BF₃OEt₃ (39.9 g, 0.281 mol, 1.3 equiv) was added dropwise over the course of 60 minutes, keeping the internal temperature below −75° C. The reaction mixture was stirred for 90 minutes at −80° C. After complete conversion was confirmed by HPLC, the reaction was slowly quenched with 2 N HCl (200 mL, 4 vol), keeping the internal temperature below −60° C. The internal temperature was adjusted to 25° C. and stirred for 30 minutes. The organic phase was collected and distilled to yield a crude residue. The residue was dissolved in toluene (300 mL, 6 vol) and washed with a 5% NaHCO₃ solution (150 mL, 3 vol) and water (150 mL, 3 vol). The resulting organic layer was concentrated under vacuum to yield a crude residue. Fractional distillation of the crude material afforded 23.2 g (55%, 99.8% HPLC purity) of the desired Compound.

### S3/J6/K8 Preparation of S3

### 2-[5-(trifluoromethyl)-3-thienyl]ethanol (S3

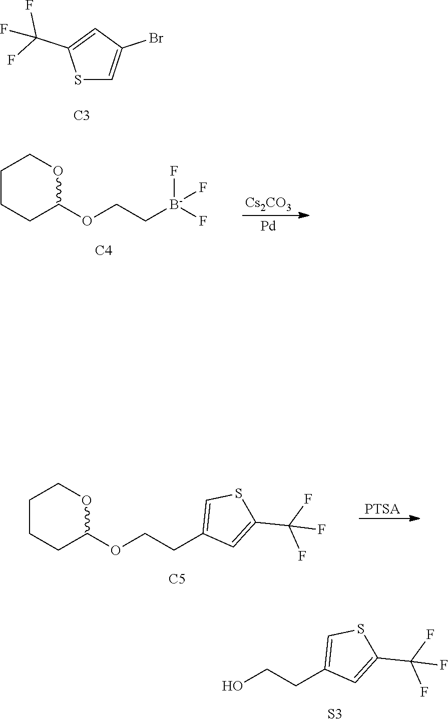

### Step 1. Synthesis of 2-[2-[5-(trifluoromethyl)-3-thienyl]ethoxy]tetra hydropyrane (C5

To a mixture of 4-bromo-2-(trifluoromethyl)thiophene C3 (9 g, 38.96 mmol), dicyclohexyl-[2-(2,6-diisopropoxyphenyl)phenyl]phosphane; methanesulfonate; N-methyl-2-phenyl-aniline; palladium (2+) (1.8 g, 2.117 mmol), and potassium trifluoro(2-tetrahydropyran-2-yloxyethyl)boranuide C4 (10 g, 42.36 mmol) was added toluene (75 mL) and water (25 mL). Nitrogen was passed over the top of the reaction before addition of Cs₂CO₃ (40 g, 122.8 mmol). A reflux condenser was added, and the reaction was heated at 100° C. for 48 hours. The reaction was diluted with EtOAc (150 mL) and water (100 mL). The two layers were separated and the aqueous layer was extracted with EtOAc (100 mL). The combined organics were washed with brine, dried over sodium sulfate, filtered, and concentrated in vacuo. Purification by silica gel chromatography (Gradient: 0-20% EtOAc in heptane) yielded the product 2-[2-[5-(trifluoromethyl)-3-thienyl]ethoxy]tetrahydropyran C5 (9 g, 82%). ¹H NMR (300 MHz, Chloroform-d) δ 7.37 (t, J=1.3 Hz, 1H), 7.22 (d, J=1.5 Hz, 1H), 4.62 (dd, J=4.2, 2.8 Hz, 1H), 3.96 (dt, J=9.6, 6.7 Hz, 1H), 3.75 (ddd, J=11.3, 8.0, 3.4 Hz, 1H), 3.62 (dt, J=9.6, 6.5 Hz, 1H), 3.55-3.41 (m, 1H), 2.93 (t, J=6.6 Hz, 2H), 1.83 (ddd, J=14.2, 6.6, 3.4 Hz, 1H), 1.73 (td, J=9.0, 4.2 Hz, 1H), 1.66-1.50 (m, 4H).

### Step 2. Synthesis of 2-[5-(trifluoromethyl)-3-thienyl]ethanol (S3

To a stirred solution of 2-[2-[5-(trifluoromethyl)-3-thienyl]ethoxy]tetrahydropyran C5 (1.8 g, 6.100 mmol) in MeOH (25 mL) was added 4-methylbenzenesulfonic acid monohydrate (1.2 g, 6.309 mmol) at room temperature. The reaction mixture was stirred at room temperature for 1 hour. The reaction mixture was diluted with water (100 mL) and extracted with MTBE (2×100 mL). The combined organic layers were washed with dilute NaHCO₃ (10 mL NaHCO₃ and 10 mL water) and brine (10 mL), dried over sodium sulfate, filtered, and evaporated under vacuum to get crude compound. Purification by silica gel chromatography (Gradient: 0-30% EtOAc in heptane) yielded the product 2-[5-(trifluoromethyl)-3-thienyl]ethanol S3 (820 mg, 69%). ¹H NMR (400 MHz, Chloroform-d) δ 7.35 (p, J=1.3 Hz, 1H), 7.23 (dt, J=1.7, 0.9 Hz, 1H), 3.85 (td, J=7.1, 6.5, 2.7 Hz, 2H), 2.87 (td, J=6.4, 0.8 Hz, 2H), 2.06 (d, J=4.3 Hz, 1H).

### Alternative Preparation of S3

### 2-[5-(trifluoromethyl)-3-thienyl]ethanol (S3

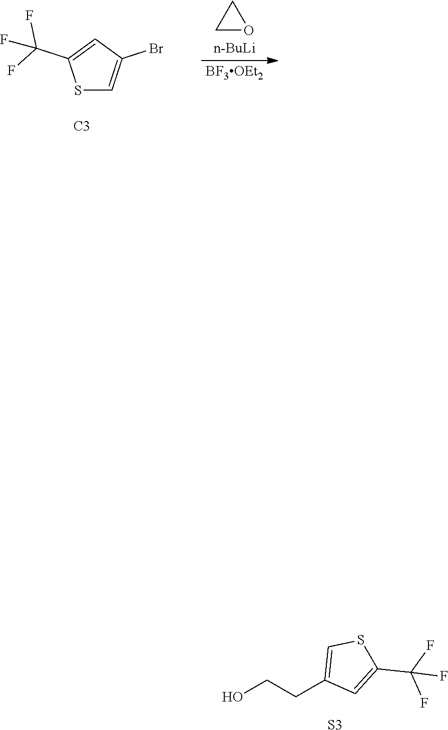

A solution of 4-bromo-2-(trifluoromethyl)thiophene C3 (50.13 g, 217.0 mmol) in Et₂O (500 mL) was cooled to −78° C. and nBuLi (91 mL of 2.48 M, 225.7 mmol) was added at a rate adapted to keep the temperature below −68° C. The reaction was stirred for 20 minutes and ethylene oxide (14 g, 317.8 mmol) was added at a rate to keep the temperature below −70° C. BF₃.OEt₂ (28 mL, 226.9 mmol) was added at a rate to keep the temperature below −68° C. The BF₃.OEt₂ addition was highly exothermic. The reaction was stirred for one hour at −78° C. and then poured into 500 mL of 1 N HCl and extracted with 500 mL of Et₂₀. The extract was dried with MgSO₄, filtered, and evaporated in vacuo. Purification by column chromatography (1600 g: isocratic gradient:10% CH₃CN-DCM) afforded 2-[5-(trifluoromethyl)-3-thienyl]ethanol S3 (22.48 g, 53%). ¹H NMR (300 MHz, Chloroform-d) δ 7.36 (t, J=1.3 Hz, 1H), 7.24 (d, J=1.5 Hz, 1H), 3.88 (q, J=6.0 Hz, 2H), 2.90 (t, J=6.3 Hz, 2H), 1.55 (t, J=5.4 Hz, 1H) ppm. 19F NMR (282 MHz, Chloroform-d) δ −55.36 ppm.

### Preparation of S23

### (3S)-3-aminobutanoic acid (S23

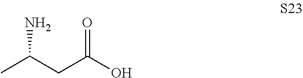

(3S)-3-aminobutanoic acid (S23) was obtained from commercial sources.

### Preparation of S25

### (4S)-4-aminopentan-2-one hydrochloride (S25

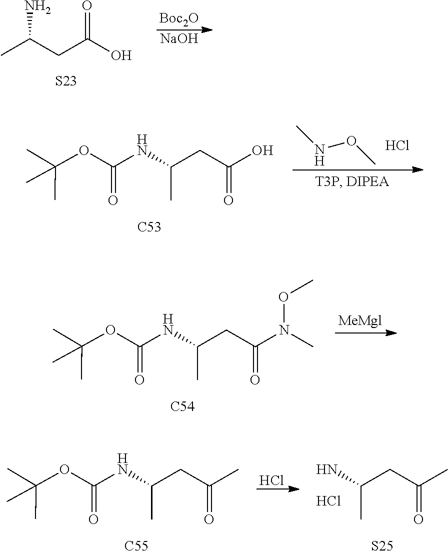

### Step 1. Synthesis of (3S)-3-(tert-butoxycarbonylamino)butanoic acid (C53

To a solution of (3S)-3-aminobutanoic acid S23 (100 g, 969.7 mmol) in dioxane (600 mL) was added aqueous NaOH solution (950 mL of 1 M, 950.0 mmol) over 15 minutes, followed by Boc₂O (300 g, 1.375 mol). The reaction mixture was stirred at room temperature for 12 hours. The reaction was partitioned with MTBE (1 L) and water (300 mL). The layers were separated, and the aqueous layer was extracted again with MTBE (500 mL). The aqueous layer was then acidified with 1 N HCl until pH=2 and extracted with DCM (3×600 mL). The combined organic layers were washed with brine, dried over MgSO₄, filtered, and concentrated in vacuo to yield (3S)-3-(tert-butoxycarbonylamino)butanoic acid C53 (176 g, 89%) as a white solid. ¹H NMR (300 MHz, Chloroform-d) δ 4.92 (s, 1H), 4.04 (s, 1H), 2.56 (dd, J=5.5, 2.9 Hz, 2H), 1.44 (s, 9H), 1.25 (d, J=6.8 Hz, 3H).

### Step 2. Synthesis of tert-butyl N-[(1S)-3-[methoxy(methyl)amino]-1-methyl-3-oxo-propyl]carbamate (C54

To a solution of (3S)-3-(tert-butoxycarbonylamino)butanoic acid C53 (160 g, 787.3 mmol) in DCM (1.5 L) was added N-methoxymethanamine (hydrochloride salt) (81 g, 830.4 mmol) followed by the addition of DIPEA (560 mL, 3.215 mol) over 10 minutes. The reaction mixture was cooled to 0° C. and T3P (600 g of 50% (w/w) in EtOAc, 942.9 mmol) was added over 45 minutes. Alternatively, other peptide coupling reagents may be used for this step. After the addition, the cooling bath was removed and the reaction was stirred at room temperature for 1 hour. The reaction mixture was cooled to 10° C. and aqueous 1 N NaOH solution (700 mL) was added and the solution stirred for 15 minutes. The organic phase was separated, washed with aqueous saturated ammonium chloride solution (200 mL) and brine (200 mL), dried, filtered through a silica plug, and concentrated in vacuo to afford tert-butyl N-[(1S)-3-[methoxy(methyl)amino]-1-methyl-3-oxo-propyl]carbamate C54 (180 g, 93%) as a clear, colorless viscous oil. ¹H NMR (300 MHz, Chloroform-d) δ 5.30 (s, 1H), 4.06 (ddd, J=14.3, 9.7, 6.0 Hz, 1H), 3.68 (s, 3H), 3.17 (s, 3H), 2.71 (dd, J=15.6, 5.2 Hz, 1H), 2.54 (dd, J=15.7, 5.7 Hz, 1H), 1.43 (s, 9H), 1.24 (d, J=6.8 Hz, 3H).

### Step 3. Synthesis of tert-butyl N-[(1S)-1-methyl-3-oxo-butyl]carbamate (C55

To a solution of tert-butyl N-[(1S)-3-[methoxy(methyl)amino]-1-methyl-3-oxo-propyl]carbamate C54 (220 g, 893.2 mmol) in THF (4 L) at 0° C. was added iodo(methyl)magnesium (900 mL of 3 M, 2.700 mol) over 40 minutes. Other sources of nucleophilc methyl, such as, e.g., MeLi and MeMgBr, may optionally be used for this step. The resulting reaction mixture was stirred at 0° C. for 4 hours. The reaction was quenched with saturated ammonium chloride solution (2 L), followed by MTBE (1 L) and water (2 L). The mixture was stirred for 30 minutes and the organic layer was separated. The aqueous phase was extracted with MTBE (1 L) and the combined organic layers were washed with saturated ammonium chloride solution (1 L), dried over MgSO₄, filtered, and concentrated in vacuo. Purification by silica gel chromatography (Gradient: 0-70% EtOAc in heptane) yielded the product tert-butyl N-[(1S)-1-methyl-3-oxo-butyl]carbamate C55 (115 g, 64%) as a white solid. ¹H NMR (300 MHz, Chloroform-d) δ 4.83 (s, 1H), 4.12-3.87 (m, 1H), 2.69 (dd, J=16.5, 5.2 Hz, 1H), 2.63-2.47 (m, 1H), 2.15 (d, J=2.3 Hz, 3H), 1.43 (d, J=2.4 Hz, 9H), 1.20 (dd, J=6.8, 2.4 Hz, 3H).

### Step 4. Synthesis of (4S)-4-aminopentan-2-one (Hydrochloride salt) (S25

To a solution of tert-butyl N-[(1S)-1-methyl-3-oxo-butyl]carbamate C55 (16.3 g, 80.18 mmol) in MeOH (30 mL) was added hydrogen chloride (50 mL of 4 M in dioxane, 200.0 mmol) over 3 minutes. Optionally, other mineral or organic acids may be used for this step. The reaction was stirred at room temperature for 5 hours and then concentrated under reduced pressure. The residue was co-evaporated with EtOH (2×30 mL) and dried under vacuum to afford (4S)-4-aminopentan-2-one (Hydrochloride salt) S25 (12 g, 98%) as a pink viscous oil. ¹H NMR (300 MHz, Chloroform-d) δ 8.06 (s, 3H), 3.48 (d, J=6.8 Hz, 1H), 2.88 (dd, J=18.0, 5.8 Hz, 1H), 2.75 (dd, J=18.0, 7.2 Hz, 1H), 2.13 (s, 3H), 1.17 (d, J=6.6 Hz, 3H).

### Preparation of S26 (Method A

### (2S,6S)-2-methyl-6-(1-methyltriazol-4-yl)piperidin-4-one (S26

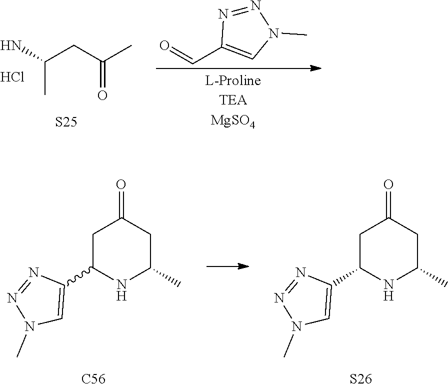

### Step 1. Synthesis of (2S)-2-methyl-6-(1-methyltriazol-4-yl)piperidin-4-one (C56

To a mixture of (4S)-4-aminopentan-2-one (Hydrochloride salt) S25 (12 g, 78.48 mmol) in EtOH (300 mL) was added 1-methyltriazole-4-carbaldehyde S17 (9 g, 81.01 mmol), L-Proline (2 g, 17.37 mmol), magnesium sulfate (12 g, 99.69 mmol), and TEA (13 mL, 93.27 mmol). The reaction mixture was stirred at room temperature overnight. The mixture was filtered and concentrated under reduced pressure. The crude residue was quenched with saturated sodium bicarbonate solution (150 mL) and extracted with DCM (3×100 mL). The combined organic layers were washed with brine (50 mL), dried over magnesium sulfate, filtered, and concentrated in vacuo. Purification by silica gel chromatography (Gradient: 0-60% of 20% MeOH/DCM in DCM) yielded the product (2S)-2-methyl-6-(1-methyltriazol-4-yl)piperidin-4-one C56 (6.7 g, 44%) as a 5:1 cis to trans ratio. Additionally, the e.r. at the stereocenter from S25 was eroded to ˜85%.

NMR for the major (cis) stereoisomer in C56: ¹H NMR (300 MHz, Chloroform-d) δ 7.47 (s, 1H), 4.26 (dd, J=10.3, 4.9 Hz, 1H), 4.11 (s, 3H), 3.17 (dqd, J=12.2, 6.2, 3.0 Hz, 1H), 2.73-2.56 (m, 2H), 2.47 (ddd, J=14.2, 3.0, 1.6 Hz, 1H), 2.21 (dd, J=14.2, 11.7 Hz, 2H), 1.28 (d, J=6.2 Hz, 3H).

NMR Rationalization of Stereoisomer Assignments in C56: Note that the major component in C56 was assigned as the cis stereoisomer using NMR coupling constant data for the peak at 4.26 ppm (C5-methylene proton). The triazole at C6 is assumed to occupy an equatorial position in the lowest energy conformation. The coupling between the axial CH at C4 and one of the CH protons at C5 (J=10.3 Hz) indicates a 180° relationship as defined by the Karplus equation. The minor trans product was removed in the subsequent re-crystallization step to afford S26.

### Step 2. Synthesis of (2S,6S)-2-methyl-6-(1-methyltriazol-4-yl)piperidin-4-one (S26

A solution of (2S)-2-methyl-6-(1-methyltriazol-4-yl)piperidin-4-one C56 (6.7 g) as a 5:1 cis to trans ratio in MTBE (100 mL) was heated to reflux for 30 minutes. Ethanol was added slowly until all solids dissolved (20 mL). The solution was refluxed for 30 minutes and allowed to slowly cool overnight. A solid crystalized out which was diluted with MTBE (30 mL), filtered, and dried under vacuum to afford (2S,6S)-2-methyl-6-(1-methyltriazol-4-yl)piperidin-4-one S26 (3.2 g, 48%) as a white solid with an enantiomeric ratio of >85%, which was carried through to all further compounds which utilized S26 as a starting material unless otherwise noted (excluding examples which were subjected to SFC purification). ¹H NMR (300 MHz, Chloroform-d) δ 7.45 (s, 1H), 4.23 (dd, J=10.3, 4.9 Hz, 1H), 4.09 (s, 3H), 3.14 (ddp, J=12.2, 6.1, 3.1 Hz, 1H), 2.71-2.52 (m, 2H), 2.44 (ddd, J=14.1, 3.0, 1.5 Hz, 1H), 2.27-2.00 (m, 2H), 1.26 (d, J=6.2 Hz, 3H).

### Alternative Preparation of S26 (Method B

### (2S,6S)-2-methyl-6-(1-methyltriazol-4-yl)piperidin-4-one (S26

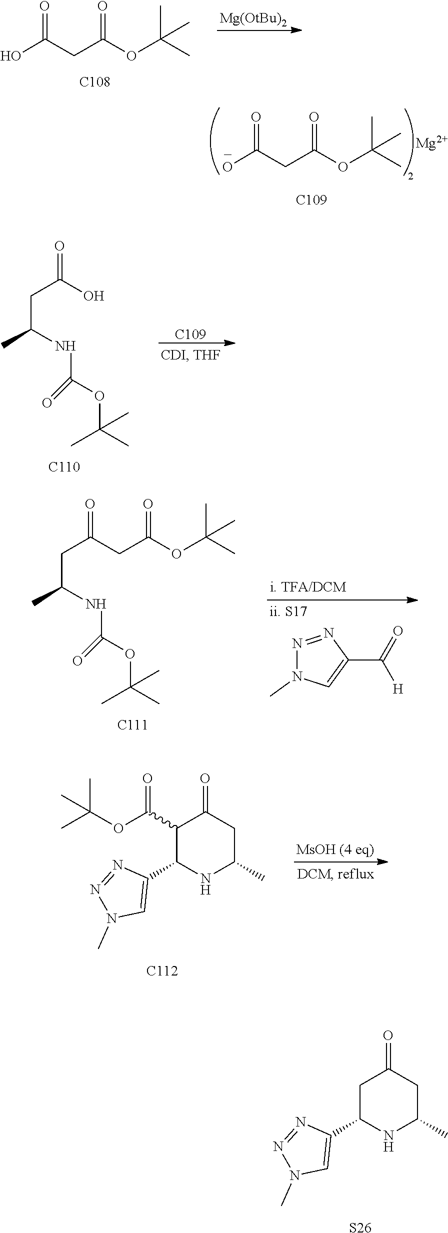

### Step 1. Synthesis of bis[(3-tert-butoxy-3-oxo-propanoyl)oxy] magnesium (C109

A solution of 3-tert-butoxy-3-oxo-propanoic acid C108 (321.51 g, 1.907 mol) in THF (2 L) was cooled to 5° C. in an ice-bath, and Mg(OEt)₂ (111.33 g, 953.5 mmol) was added. The reaction was stirred for 30 minutes at 0° C., removed from the cooling bath, and stirred at room temperature overnight. The reaction was filtered over a plug of Celite®, and the plug was washed with additional THF. The clear, colorless filtrate was evaporated in vacuo to afford a mushy solid. The solid was triturated with 1 L of diethyl ether and filtered. The filter-cake was washed with Et₂O and dried in vacuo. The filtrate was evaporated in vacuo again and was then triturated with a small volume of Et₂O and filtered to afford a second crop of the product. The crops were combined and dried in vacuo to afford bis[(3-tert-butoxy-3-oxo-propanoyl)oxy]magnesium C109 (294.49 g, 90%) as a white solid. ¹H NMR (300 MHz, Methanol-d₄) δ 4.92 (s, 4H), 1.48 (s, 18H) ppm.

### Step 2. Synthesis of tert-butyl (5S)-5-(tert-butoxycarbonylamino)-3-oxo-hexanoate (C111

To a solution of (3S)-3-(tert-butoxycarbonylamino)butanoic acid C110 (170.15 g, 837.2 mmol) in THF (1.5 L) was added CDI (149.8 g, 923.8 mmol). The milky suspension cleared over the next few minutes. Gas evolution was observed. The reaction was stirred for 3 hours at room temperature. Bis[(3-tert-butoxy-3-oxo-propanoyl)oxy]magnesium C109 (172.19 g, 502.6 mmol) was added. Another milky suspension was formed that cleared after stirring for 30 minutes. The reaction was stirred for 48 hours. The reaction was poured into 1.5 L of 1 N HCl and extracted with MTBE (1 L). The pH was confirmed to be approximately pH 3. The extract was washed with saturated aqueous NaHCO₃, separated, dried with MgSO₄, filtered, and evaporated in vacuo to afford tert-butyl (5S)-5-(tert-butoxycarbonylamino)-3-oxo-hexanoate C111 (248.5 g, 98.5%). ¹H NMR (300 MHz, Chloroform-d) δ 4.90 (d, J=18.1 Hz, 1H), 4.04 (dt, J=13.8, 6.6 Hz, 1H), 3.47-3.22 (m, 2H), 2.76 (qd, J=17.0, 5.7 Hz, 2H), 1.48 (s, 9H), 1.44 (s, 9H), 1.23 (d, J=6.8 Hz, 3H) ppm.

### Step 3. Synthesis of tert-butyl (2S,3R,6S)-6-methyl-2-(1-methyltriazol-4-yl)-4-oxo-piperidine-3-carboxylate (C112

To a solution of tert-butyl (5S)-5-(tert-butoxycarbonylamino)-3-oxo-hexanoate C111 (248.5 g, 824.5 mmol) in DCM (1.5 L) was added TFA (240 mL, 3.115 mol), and the reaction was stirred overnight. The reaction was evaporated in vacuo at 25° C. The solid that remained was triturated with 500 mL of pentane and filtered. The filter-cake was washed with pentane, and most of the solvent was pulled off of the filter-cake. The cake was transferred back to the reaction flask and dissolved in 1 L of DCM.

1-methyltriazole-4-carbaldehyde S17 (120.7 g, 1.086 mol) was added. The reaction was stirred at room temperature overnight. Brine (100 mL) was added and 6 N NaOH was added until the aqueous layer remained alkaline when the funnel was shaken. The organic layer was isolated, and the aqueous layer was extracted with DCM (1 L). The organic layers were combined, dried with MgSO₄, and filtered over a plug of silica gel. The plug was eluted with 10% MeOH in EtOAc. The filtrate was evaporated in vacuo to afford a solid that was triturated with MTBE (500 mL) and filtered. The filter-cake was washed with MTBE and dried in vacuo to give a crop of product. The mother liquor from the trituration was concentrated. The solid that precipitated was filtered to provide a second crop of the product. The crops were combined to give (2S,3R,6S)-6-methyl-2-(1-methyltriazol-4-yl)-4-oxo-piperidine-3-carboxylate C112 (105.45 g, 43%). ¹H NMR (300 MHz, Chloroform-d) δ 7.48 (s, 1H), 4.52 (d, J=11.0 Hz, 1H), 4.09 (s, 3H), 3.61 (dd, J=11.0, 1.0 Hz, 1H), 3.21 (ddd, J=11.7, 6.1, 2.9 Hz, 1H), 2.55 (dd, J=13.7, 2.9 Hz, 1H), 2.37-2.13 (m, 1H), 1.98 (s, 1H), 1.39 (s, 9H), 1.29 (d, J=6.3 Hz, 3H) ppm.

### Step 4. Synthesis of (2S,6S)-2-methyl-6-(1-methyltriazol-4-yl)piperidin-4-one (S26

To a solution of tert-butyl (2S,3R,6S)-6-methyl-2-(1-methyltriazol-4-yl)-4-oxo-piperidine-3-carboxylate C112 (70.59 g, 239.8 mmol) in DCM (750 mL) was added MsOH (62 mL, 955.4 mmol), and the reaction was heated to reflux for 6 hours. The reaction was cooled and poured into a separatory funnel. Brine (approx. 100 mL) was added. 6 N NaOH was added until the aqueous layer remained alkaline after shaking. The organic layer was separated and the aqueous layer was extracted with DCM (2×500 mL). The organic layers were combined, dried with MgSO₄, filtered, and evaporated in vacuo to afford (2S,6S)-2-methyl-6-(1-methyltriazol-4-yl)piperidin-4-one S26 (43.74 g, 94%). ¹H NMR (300 MHz, Chloroform-d) δ 7.46 (s, 1H), 4.20 (dd, J=10.1, 5.1 Hz, 1H), 4.06 (s, 3H), 3.11 (dqd, J=12.3, 6.2, 3.0 Hz, 1H), 2.73-2.48 (m, 2H), 2.40 (ddd, J=14.1, 3.0, 1.5 Hz, 1H), 2.25-2.00 (m, 2H), 1.23 (d, J=6.2 Hz, 3H) ppm.

### Preparation of L2

### (2'S,6'S, 7S)-2′-methyl-6′-(1-methyltriazol-4-yl)-2-(trifluoromethyl)spiro[4,5-dihydrothieno[2,3-c]pyran-7,4′-piperidine] (L2

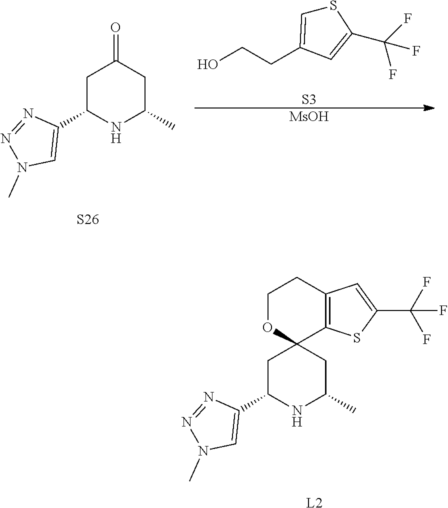

To a solution of (2S,6S)-2-methyl-6-(1-methyltriazol-4-yl)piperidin-4-one S26 (250 mg, 1.287 mmol) and 2-[5-(trifluoromethyl)-3-thienyl]ethanol S3 (350 mg, 1.748 mmol) in DCM (5 mL) was added MsOH (500 μL, 7.705 mmol) and the reaction was heated to 40° C. After 16 hours, additional MsOH (200 μL, 3.082 mmol) was added, and the reaction continued heating overnight. The mixture was diluted with water (4 mL) and DCM (5 mL) and quenched with aqueous NaOH (2 mL of 6 M, 12.00 mmol). The mixture was separated, extracted with DCM (2×5 mL), passed over a phase separator, and the organics concentrated in vacuo. Purification by silica gel chromatography (Gradient: 0-10% MeOH in DCM) yielded (2'S,6'S,7S)-2′-methyl-6′-(1-methyltriazol-4-yl)-2-(trifluoromethyl)spiro[4,5-dihydrothieno[2,3-c]pyran-7,4′-piperidine] L2 (445 mg, 93%) as a white solid. Note that relative stereochemistry in Compound L2 was assigned through NOE NMR studies. ¹H NMR (300 MHz, Chloroform-d) δ 7.46 (s, 1H), 7.14 (s, 1H), 4.47 (d, J=11.6 Hz, 1H), 4.08 (d, J=3.3 Hz, 3H), 4.00 (s, 2H), 3.36 (s, 1H), 2.72 (d, J=5.6 Hz, 2H), 2.41 (d, J=14.2 Hz, 1H), 2.12 (d, J=13.7 Hz, 1H), 1.86 (t, J=12.7 Hz, 1H), 1.49 (d, J=12.8 Hz, 1H), 1.15 (d, J=6.3 Hz, 3H). LCMS m/z 373.07 [M+H]⁺.

### Preparation of S32

### (2S,4S,6S)-2-methyl-6-(1-methyltriazol-4-yl)-1-(2,2,2-trifluoroacetyl)-2′-(trifluoromethyl)spiro[piperidine-4,7′-thieno[2,3-c]pyran]-4′-one (S32

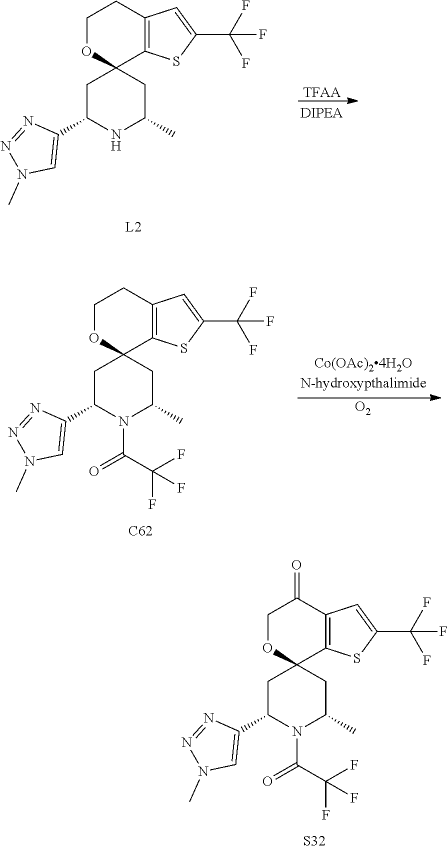

### Step 1. Synthesis of 2,2,2-trifluoro-1-[(2'S,6'S,7S)-2′-methyl-6′-(1-methyltriazol-4-yl)-2-(trifluoromethyl)spiro[4,5-dihydrothieno[2,3-c]pyran-7,4′-piperidine]-1′-yl]ethanone (C62

To a solution of (2'S,6'S,7S)-2′-methyl-6′-(1-methyltriazol-4-yl)-2-(trifluoromethyl)spiro[4,5-dihydrothieno[2,3-c]pyran-7,4′-piperidine] L2 (1260 mg, 3.352 mmol) dissolved in DCM (25 mL) cooled to −15° C. was added DIPEA (800 μL, 4.593 mmol), followed by TFAA (550 μL, 3.957 mmol). After 5 minutes, the mixture was quenched with 1 N HCl (25 mL) and the phases were separated. The organic layer was dried with MgSO₄, filtered, and concentrated. Purification by silica gel chromatography (Gradient: 0-50% EtOAc in Heptane) yielded 2,2,2-trifluoro-1-[(2'S,6'S,7S)-2′-methyl-6′-(1-methyltriazol-4-yl)-2-(trifluoromethyl)spiro[4,5-dihydrothieno[2,3-c]pyran-7,4′-piperidine]-1′-yl]ethanone C62 (1444 mg, 90%). ¹H NMR (400 MHz, Methanol-d₄) δ 7.93 (s, 1H), 7.27 (d, J=1.3 Hz, 1H), 5.63 (s, 1H), 4.46 (h, J=7.1 Hz, 1H), 4.11 (d, J=1.4 Hz, 3H), 3.96 (td, J=5.6, 1.7 Hz, 2H), 3.04 (s, 1H), 2.79-2.70 (m, 3H), 2.51 (s, 1H), 2.09 (dd, J=14.7, 7.3 Hz, 1H), 1.23 (q, J=9.6, 8.4 Hz, 3H). LCMS m/z 469.14 [M+H]⁺.

### Step 2. Synthesis of (2S,4S,6S)-2-methyl-6-(1-methyltriazol-4-yl)-1-(2,2,2-trifluoroacetyl)-2′-(trifluoromethyl)spiro[piperidine-4,7′-thieno[2,3-c]pyran]-4′-one (S32

To a mixture of 2,2,2-trifluoro-1-[(2'S,6'S,7S)-2′-methyl-6′-(1-methyltriazol-4-yl)-2-(trifluoromethyl)spiro[4,5-dihydrothieno[2,3-c]pyran-7,4′-piperidine]-1′-yl]ethanone C62 (708 mg, 1.511 mmol) in acetonitrile (10 mL) was added N-hydroxyphthalimide (165 mg, 1.011 mmol) and cobaltous diacetate tetrahydrate (35 mg, 0.1405 mmol), and then the mixture was vacuum purged with an oxygen balloon three times. The mixture was heated to 60° C. and stirred. After an hour and a half, the reaction was cooled to room temperature. The mixture was vacuum purged with nitrogen three times and then diluted with MTBE (25 mL) and saturated aqueous bicarbonate (25 mL). The layers were separated, and the organic layer was washed with aqueous NaHCO₃ (2×50 mL) and brine (50 mL). The organic layer was dried with Na₂SO₄, filtered, and concentrated. Purification by silica gel chromatography (Gradient: 0-50% EtOAc in heptane) afforded (2S,4S,6S)-2-methyl-6-(1-methyltriazol-4-yl)-1-(2,2,2-trifluoroacetyl)-2′-(trifluoromethyl)spiro[piperidine-4,7′-thieno[2,3-c]pyran]-4′-one S32 (207 mg, 26%), ¹H NMR (300 MHz, Methanol-d₄) δ 7.98 (s, 1H), 7.80 (d, J=1.4 Hz, 1H), 5.70 (s, 1H), 4.48 (s, 1H), 4.45 (s, 2H), 4.12 (s, 3H), 2.95 (dd, J=14.8, 9.8 Hz, 1H), 2.73 (s, 1H), 2.22 (dd, J=14.8, 8.4 Hz, 1H), 1.29 (s, 1H), 1.19 (d, J=14.9 Hz, 3H). LCMS m/z 483.45 [M+H]⁺.

### 2. Synthesis of Compound I Phosphate Salt Hydrate Form A

### (2S,4S,4'S,6S)-2-methyl-6-(1-methyl-1H-1,2,3-triazol-4-yl)-2′-(trifluoromethyl)-4′,5′-dihydrospiro[piperidine-4,7′-thieno[2,3-c]pyran]-4′-ol (Compound I) Phosphate Salt Hydrate

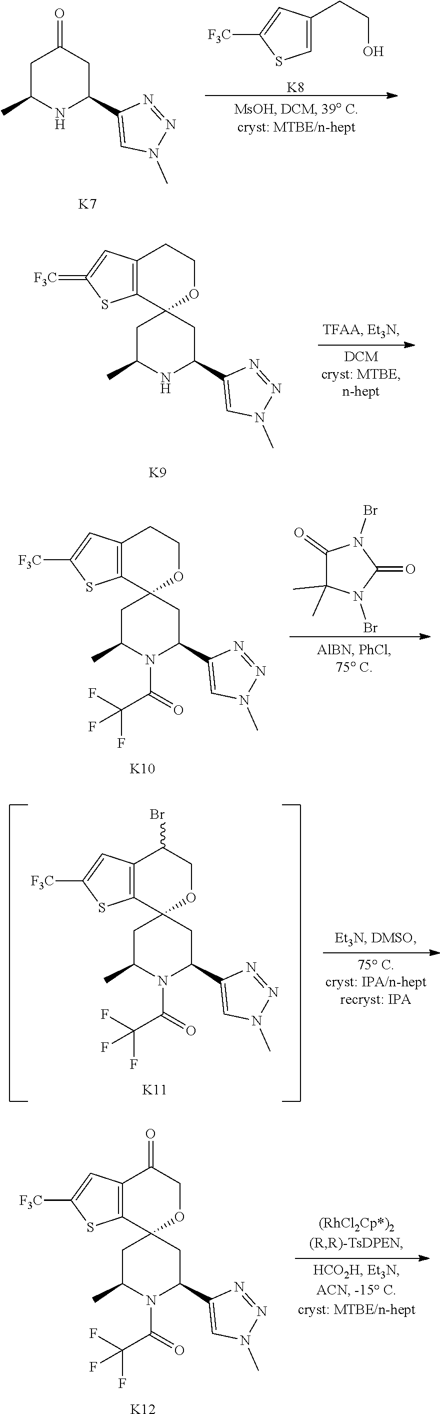

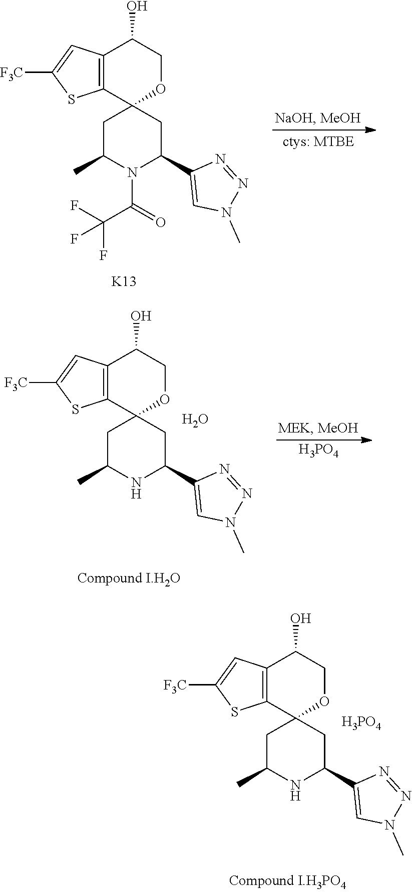

Step 1. A solution of K7 (70 g, 0.360 mol, 1.0 equiv) and 2-[5-trifluoromethyl)-3-thienyl]ethanol K₈ (74.2 g, 0.378 mol, 1.05 equiv) in dichloromethane (210 mL, 3 vol) was cooled to 5° C. Methanesulfonic acid (210.6 mL, 3.24 mol, 9 equiv) was charged to the reactor while maintaining an internal temperature of less than 30° C. Optionally, other organic or mineral acids may be used for this step. The resulting reaction mixture was heated to 39° C. After 18 hours, HPLC analysis indicated greater than 99% conversion to K₉. The reaction mixture was cooled to 30° C., charged with dichloromethane (280 mL, 4 vol), and further cooled to 0° C. The pH was adjusted to pH 10 with 4 N sodium hydroxide (830 mL). The organic layer was separated, and the aqueous phase was back-extracted with DCM (350 mL, 5 vol). The combined organics were washed with water (350 mL, 5 vol) and concentrated at reduced pressure to 3.5 total volumes. The batch was charged with MTBE (5 vol) and concentrated under reduced pressure to 3.5 total volumes. This put/take cycle was repeated three additional times, and the resulting 3.5 vol. mixture was diluted with MTBE (6.5 vol) to provide a 10 vol. mixture. The slurry was heated to 50° C. and stirred for 5 hours, then charged with n-heptane (700 mL, 10 vol) over 2 hours. The resulting suspension was cooled to 20° C. over 5 hours and stirred for 18 hours. The suspension was filtered, washed with 1:2 MTBE/n-heptane (2×140 mL, 2×2 vol), and dried under vacuum while flushing with nitrogen at 50° C. for 18 hours to give 103 g of K₉ (77% yield).

Step 2. A solution of K9 (50 g, 0.134 mol, 1.0 equiv) and triethylamine (22.5 mL, 0.161 mol, 1.2 equiv) in dichloromethane (380 mL, 7.6 vol) was cooled to 5° C. Alternatively, other amine bases may be employed for this step. At 5° C., trifluoroacetic acid anhydride (20.5 mL, 0.148 mol, 1.1 equiv) was charged to the reactor while keeping the internal temperature below 15° C. The resulting reaction mixture was stirred at 5° C. for 1 hour, at which time HPLC showed 99.8% conversion to K₁₀. The reaction mixture was charged at 5° C. with water (200 mL, 4 vol). The organic layer was separated and sequentially washed with 5% NaHCO₃ (200 mL, 4 vol), 2 N HCl (2×200 mL, 2×4 vol), and water (2×200 mL, 2×4 vol). The organic layer was concentrated under reduced pressure to 3.5 total volumes. MTBE (400 mL, 8 vol) was charged, and the batch was concentrated under reduced pressure to 3.5 vol. This put/take cycle was repeated two additional times, and the mixture was concentrated to 3 volumes after the final cycle. The solution was heated to 40° C. and charged with n-heptane (190 mL, 2 vol) over 1 hour. The batch was cooled to 20° C. over 2 hours to yield a suspension. n-Heptane (500 mL, 10 vol) was charged over 2 hours, and the resulting suspension was stirred for 18 hours. The suspension was filtered, washed with 5% MTBE/n-heptane (2×125 mL, 2×2.5 vol), and dried under vacuum while flushing with nitrogen at 50° C. for 18 hours to give 53 g of K10 (84% yield).

Steps 3 and 4. A suspension of K10 (70 g, 149.4 mmol, 1.0 equiv), and 1,3-dibromo-5,5′-dimethylhydantoin (29.9 g, 104.6 mmol, 0.7 equiv) in anhydrous chlorobenzene (280 L, 4 vol) was sparged with sub-surface nitrogen bubble for 60 minutes. Alternatively, other brominating agents, such as, e.g., NBS, may be used for this step. Bromination may also be effected using catalytic ZrCl₄ or ZrBr₄, instead of AIBN, in dichloromethane and other solvents, which allows for a potential decrease in temperature down to 0° C. and removal of AIBN, which is a thermal hazard liability as it has a low thermal onset temperature. The mixture was heated to 75° C. and charged at that temperature with a prepared solution of azobisisobutyronitrile (0.49 g, 3 mmol, 0.02 equiv) in anhydrous chlorobenzene (70 mL, 1 vol) over 60 minutes. This step may also be carried out at 50° C. After stirring for 2 hours at 75° C., HPLC analysis showed conversion to K11. The reaction mixture was cooled to 60° C. and charged with anhydrous, degassed DMSO (350 mL, 5 vol) over 30 minutes, followed by anhydrous, degassed triethylamine (104 mL, 747 mmol, 5 equiv) over 30 minutes. Optionally, other amine bases may be used to affect this transformation. The reactor headspace was well purged with nitrogen, and the batch was heated to 75° C. After 15 hours, HPLC analysis showed >99% conversion of K11 to K₁₂. The batch was cooled to 20° C. and diluted with dichloromethane (210 mL, 3 vol). The batch was further cooled to 5° C. and charged with water (350 mL, 5 vol) while keeping the solution temperature below 30° C. The organic layer was separated, and the aqueous layer was back-extracted with dichloromethane (210 mL, 3 vol). The organic phases were combined and washed sequentially with 2 N HCl (350 mL, 5 vol) and water (2×350 mL, 2×5 vol). The organic phase was concentrated under reduced pressure to 3 total volumes. The solution was charged with IPA (560 mL, 8 vol) and concentrated under reduced pressure to 3 volumes. This put/take cycle was repeated two additional times, giving a 3-volume solution that was further diluted with IPA (70 mL, 1 vol). The resulting 4 vol mixture was heated to 75° C. to provide a homogenous solution and then cooled to 50° C. The solution was seeded (0.1 wt %) at 50° C., stirred for 1 hour, and further cooled to 20° C. over 2 hours. After stirring an additional 18 hours at 20° C., the slurry was charged with n-heptane (70 mL, 1 vol) over 1 hour. The slurry was stirred for 4 hours at 20° C., filtered, washed with 1:1 IPA/n-heptane (2×70 mL, 2×2 vol), and dried under vacuum while flushing with nitrogen at 50° C. for 18 hours to give 31.2 g of K12 (43% yield from K10). The dried K12 was suspended in IPA (93 mL, 3 vol), heated to 80° C., and stirred at that temperature for 2 hours. The solution was cooled to 70° C. over 1 hour and stirred for 1 hour. The suspension was cooled to 20° C. over 5 hours and stirred at that temperature for 18 hours. The suspension was filtered, washed with 1:1 IPA/n-heptane 2×35 mL, 2×0.5 vol), and dried under vacuum while flushing with nitrogen at 50° C. for 18 hours to give 28.8 g of K12 (40% yield from K10).

Step 5. The pentamethylcyclopentadienylrhodium(III)chloride dimer (154 mg, 0.002 eq) and (R,R)-TsDPEN (182 mg, 0.004 eq) were combined in acetonitrile (240 mL, 4 vol), and the mixture was sparged with nitrogen while stirring at 20° C. for 1 hour. The mixture was cooled to −15° C. and a prepared mixture of formic acid (27.0 mL, 5.5 equiv) and triethylamine (38.1 mL, 2.2 eq) was added over 30 minutes and the resultant red/orange solution was stirred for 15 minutes at −15° C. A solution of K12 (60 g, 1.0 equiv) in acetonitrile (240 mL, 4 vol) was separately prepared and added to the cold catalyst solution over 45 minutes. Alternatively, a solution of K12, pentamethylcyclopentadienylrhodium(III)chloride dimer and (R,R)-TsDPEN in acetonitrile may be prepared and charged with a prepared solution of formic acid and triethylamine. The mixture was sparged with subsurface nitrogen bubble for 15 minutes, stirred at −15° C. for 20 hours, warmed to 0° C., and stirred for an additional 20 hours. The temperature was adjusted to 20° C. and the mixture was charged with MTBE (360 mL, 6 vol) and 18% NaCl (aq) (360 mL, 6 vol). The phases were mixed, and the phases separated. The organic phase was washed sequentially with 18% NaCl (aq) (2×360 mL, 6 vol), 4% NaHCO₃(aq) (360 mL, 6 vol), and 18% NaCl (aq.) (180 mL, 3 vol). The reaction solution was concentrated to 3 total volumes under reduced pressure, then solvent swapped to MTBE by adding MTBE (360 mL, 6 vol) and concentrating to 3 volumes under reduced pressure. This put/take cycle was repeated 3 additional times. The resulting solution was diluted to 4 total volumes with MTBE and charged with DCM (240 mL, 4 vol) and MTBE-pre-washed SiliaMetS DMT resin (30 g, 50 wt %). The mixture was stirred vigorously at 20° C. for 2 hours. The resin slurry was filtered under vacuum. The reaction flask was rinsed with a solution of 2:1 DCM:MTBE (120 mL, 2 vol) and the rinse was transferred to the resin. The resulting slurry was mixed, then filtered under vacuum. The resin was rinsed once more with a solution of 2:1 DCM:MTBE (120 mL, 2 vol) by adding it to the resin, mixing, then filtering under vacuum. The rinses and original filtrate were combined and transferred back to the reaction flask using 2:1 DCM:MTBE (30 mL, 0.5 vol) as a final rinse after the transfer. The filtrate was combined with MTBE-pre-washed SiliaMetS DMT resin (30 g, 50 wt %) and stirred vigorously for 2 hours at 20° C. The resin slurry was under vacuum. A solution of 2:1 DCM:MTBE (120 mL, 2 vol) was used to rinse the reaction flask, and the rinse was transferred to the resin in the frit. The slurry was mixed and filtered under vacuum. A solution of 2:1 DCM:MTBE (120 mL, 2 vol) was charged to the resin in the frit and the slurry was mixed, then filtered under vacuum. The combined filtrates were transferred back to the reaction flask using 2:1 DCM:MTBE (30 mL, 0.5 vol) as a rinse. The filtrate was combined with MTBE-pre-washed SiliaMetS DMT resin (30 g, 50 wt % loading) and stirred vigorously for 18 hours. The resultant resin slurry was filtered under vacuum. The reaction flask was rinsed with a solution of 2:1 DCM:MTBE (120 mL, 2 vol). The rinse was added to the resin in the frit, and the slurry was mixed then filtered under vacuum. A solution of 2:1 DCM:MTBE (120 mL, 2 vol) was added to the resin in the frit, and the slurry was mixed then filtered under vacuum. The SiliaMetS DMT may be replaced with Florisil or other filter aids, resins or activated carbons. The combined filtrate was transferred to a flask, then concentrated to 3 total volumes (180 mL) of solution. MTBE (480 mL, 8 vol) was added, and the solution was concentrated to 3 total volumes (180 mL). This put/take cycle was repeated two additional times. The resulting solution was diluted to 5 vol (300 mL) with MTBE, heated to 50° C. and stirred for 3 hours. n-Heptane (240 mL, 4 vol) was added over 60 minutes, and the slurry was maintained at 50° C. for an additional 1 hour. The slurry was cooled to 20° C. over 3 hours and stirred overnight. The slurry was filtered under vacuum. The cake was rinsed with 1:1 MTBE:heptane (2×60 mL, 2×1 vol), and the solids were dried under vacuum at 50° C. for 18 hours to yield 58.5 g of K13 (83% yield).

Step 6. K13 (43.5 g, 89 mmol, 1 equiv) and methanol (150.0 mL, 3 vol) were combined and agitated until full dissolution was observed. 6 N NaOH (89 mL, 6 eq) was added drop-wise over 30 minutes, and the mixture was heated to 60° C. and stirred for 1 hour at which time complete conversion to Compound I was achieved. Optionally, other metal hydroxides like LiOH, KOH or CsOH could be used for this step. The reaction solution was cooled to 15° C. and treated with isopropyl acetate (250 mL, 5.75 vol). Water (100 mL, 2.3 vol) was then added, and the mixture was agitated for 30 minutes. The phases were separated, and the aqueous phase was back-extracted with isopropyl acetate (250 mL, 5.75 vol). The organics were combined and washed with 10% NaCl (aq.) (2×250 mL, 2×5.75 vol) and water (250 mL, 5.75 vol). The organics were concentrated to 4.0 total volumes (174 mL). The solution was charged with MTBE (11.5 vol, 500 mL) and concentrated again to 4.0 vol. This put/take cycle was repeated three additional times. MTBE (75 mL, 1.75 vol) was added to give a 5.75 vol, 250 mL, solution. While stirring at 20° C., water (3.2 mL, 180 mmol, 2 eq) was added over 2 hours, inducing crystallization. The slurry was stirred at 20° C. for 1 hour, then heated to 50° C. and stirred at that temperature for 3 hours. The suspension was cooled to 20° C. and stirred for 18 hours. The slurry was filtered under vacuum, and the cake was washed with MTBE (100 mL, 2.3 vol). The solids were dried at 50° C. under vacuum for 18 hours to provide 29 g of Compound I free form Monohydrate (Compound I.H₂O) (81% yield).

Step 7. Method A. 1 eq. of Compound I free form monohydrate was charged to a reactor followed by 6 vol. of MEK. Agitation was started at 20° C. Once a clear solution was obtained, the solution was polish filtered and charged back to the reactor. Water (0.2 vol.) was added to the clear solution and agitation continued. 1 wt % of Compound I phosphate salt was added as seeds. In a separate container, 1.02 eq. of 85 wt % phosphoric acid was diluted with 3.8 vol. of MEK. This phosphoric acid solution was then added to the reactor slowly over 3 hours. The final slurry was agitated at 20° C. for 2 hours, then filtered under vacuum. The resulting wet cake was washed with 3 vol. of MEK. The wet cake was dried under vacuum with a nitrogen bleed at 80° C. to yield Compound I Phosphate Salt Hydrate Form A (Compound I.H₃PO₄) (about 90% yield).

Method B. 1 eq. of Compound I free form monohydrate was charged to a reactor, followed by 6 vol. of MEK. Agitation was commenced at 20° C., and once a clear solution was obtained, the solution was polish filtered and charged back to the reactor. Water (0.2 vol.) was added to the clear solution and agitation continued. In a separate container, 1.02 eq. of 85 wt % phosphoric acid was diluted with 3.8 vol. of MEK. This phosphoric acid solution was then added to the reactor slowly over 3 hours. The final slurry was agitated at 20° C. for 2 hours then filtered under vacuum. The resulting wet cake was washed with 3 vol. of MEK. The wet cake was dried under vacuum with a nitrogen bleed at 80° C. to yield Compound I Phosphate Salt Hydrate Form A (Compound I.H₃PO₄) (about 90% yield).

Note: Compound I Phosphate Salt Hydrate Form A is a crystalline hydrate.

### 3. Synthesis of Compound I (Amorphous

### (2S,4S,4'S,6S)-2-methyl-6-(1-methyl-1H-1,2,3-triazol-4-yl)-2′-(trifluoromethyl)-4′,5′-dihydrospiro[piperidine-4,7′-thieno[2,3-c]pyran]-4′-ol (Compound I), Amorphous Form

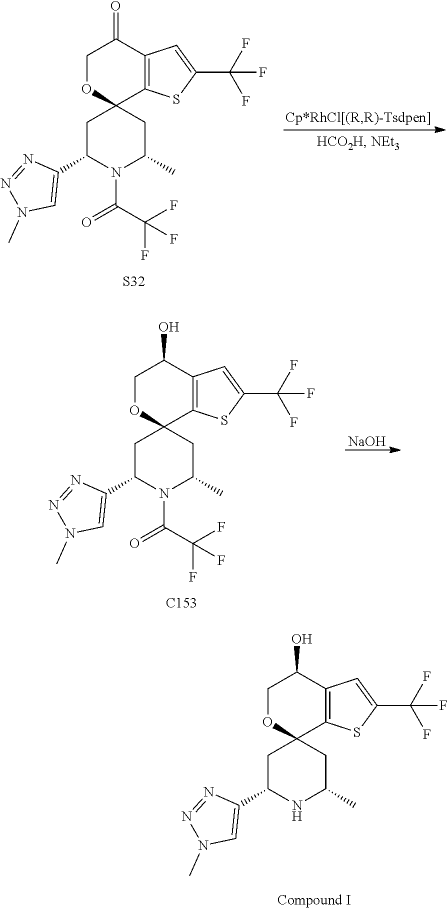

### Step 1. Synthesis of 2,2,2-trifluoro-1-[(2'S,4S,6'S, 7S)-4-hydroxy-2′-methyl-6′-(1-methyltriazol-4-yl)-2-(trifluoromethyl)spiro[4,5-dihydrothieno[2,3-c]pyran-7,4′-piperidine]-1′-yl]ethenone (C153

To (2S,4S,6S)-2-methyl-6-(1-methyltriazol-4-yl)-1-(2,2,2-trifluoroacetyl)-2′-(trifluoromethyl)spiro[piperidine-4,7′-thieno[2,3-c]pyran]-4′-one S32 (2.23 g, 4.63 mmol) in DCM (20 mL) was added a solution of 1,2,3,4,5 pentamethylcyclopentane rhodium (2+) tetrachloride (7 mg, 0.002 mmol) and N-[(1R,2R)-2-amino-1,2-diphenyl-ethyl]-4-methyl-benzenesulfonamide (8.5 mg, 0.005 mmol) in DCM (2 mL), followed by a solution of formic acid (0.9 mL, 5.15 mmol) and triethylamine (1.3 mL, 2.01 mmol). The flask was fitted with an empty balloon to capture the CO₂ off-gas byproduct. After three hours, the mixture was washed with saturated aqueous sodium bicarbonate (10 mL). The organic phase was separated, passed through a phase separator, and concentrated. Silica gel purification (Column: 40 g silica gel, Gradient: 0-50% EtOAc in Heptane) afforded 2,2,2-trifluoro-1-[(2'S,4S,6'S,7S)-4-hydroxy-2′-methyl-6′-(1-methyltriazol-4-yl)-2-(trifluoromethyl)spiro[4,5-dihydrothieno[2,3-c]pyran-7,4′-piperidine]-1′-yl]ethenone C153 (2.27 g, 100%) as a white solid. LCMS m/z 485.11 [M+H]⁺.

### Step 2. Synthesis of (2'S,4S,6'S, 7S)-2′-methyl-6′-(1-methyltriazol-4-yl)-2-(trifluoromethyl)spiro[4,5-dihydrothieno[2,3-c]pyran-7,4′-piperidine]-4-ol (Compound I

To a solution of 2,2,2-trifluoro-1-[(2'S,4S,6'S,7S)-4-hydroxy-2′-methyl-6′-(1-methyltriazol-4-yl)-2-(trifluoromethyl)spiro[4,5-dihydrothieno[2,3-c]pyran-7,4′-piperidine]-1′-yl]ethenone C153 (2.27 g, 4.63 mmol) in MeOH (45 mL) was added NaOH (8 mL of 6 M, 48.00 mmol), and the mixture was stirred at 60° C. After 75 minutes, the mixture was diluted with saturated aqueous ammonium chloride until pH 10 (about 40 mL), water (40 mL), and extracted with MTBE (100 mL). The aqueous layer was extracted with additional MTBE (2×50 mL), and the combined organic layers were washed with saturated aqueous NaCl, dried over MgSO₄, and concentrated to give amorphous (2'S,4S,6'S,7S)-2′-methyl-6′-(1-methyltriazol-4-yl)-2-(trifluoromethyl)spiro[4,5-dihydrothieno[2,3-c]pyran-7,4′-piperidine]-4-ol I (1.84 g, 88%). ¹H NMR (400 MHz, Chloroform-d) δ 7.48 (s, 1H), 7.39 (q, J=1.2 Hz, 1H), 4.58 (d, J=8.0 Hz, 1H), 4.44 (dd, J=11.7, 2.5 Hz, 1H), 4.09 (s, 4H), 4.01 (dd, J=12.5, 2.7 Hz, 1H), 3.43 (ddd, J=11.2, 6.4, 2.5 Hz, 1H), 2.48 (dt, J=13.8, 2.6 Hz, 1H), 2.16-2.07 (m, 2H), 1.77 (dd, J=13.9, 11.8 Hz, 1H), 1.63 (s, 1H), 1.28 (s, 1H), 1.18 (d, J=6.3 Hz, 3H). LCMS m/z 389.09 [M+H]⁺.

### Example 2: Solid Forms of Compound I

### Solid State NMR Experimental—Applies to all Solid Forms of Compound I Disclosed Herein

A Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using 1H MAS Ti saturation recovery relaxation experiment in order to set up proper recycle delay of the ¹³C and ³¹P cross-polarization (CP) MAS experiments. The fluorine relaxation time was measured using ¹⁹F MAS Ti saturation recovery relaxation experiment in order to set up proper recycle delay of the ¹⁹F MAS experiment. The CP contact time of carbon as well as phosphorus CPMAS experiments was set to 2 ms. A CP proton pulse with linear ramp (from 50% to 100%) was employed. The carbon Hartmann-Hahn match was optimized on external reference sample (glycine), while phosphorus Hartmann-Hahn match was optimized on the actual samples. All carbon, phosphorus and fluorine spectra were recorded with proton decoupling using TPPM15 decoupling sequence with the field strength of approximately 100 kHz.

1. Compound I Phosphate Salt Methanol Solvate

### A. Synthetic Procedure

Amorphous Compound I (50 mg) was added to MEK (0.3 mL). To this was added 0.27 mL of a 0.5 M stock solution of H₃PO₄ in MeOH. The sample was left at ambient temperature overnight. The solids were filtered using a 0.22 μm PVDF Eppendorf filter tube and washed with 4:1 n-Heptane/MEK (v/v) that was chilled over ice. Subsequent washes were performed with n-Heptane, resulting in a solid white powder. XRPD of the wet material showed the product was Compound I Phosphate Salt Methanol Solvate.

### B. X-Ray Powder Diffraction

The powder, X-ray powder diffraction (XRPD), diffractogram of Compound I Phosphate Salt Methanol Solvate was acquired at room temperature in transmission mode using a PANalytical Empyrean system equipped with a sealed tube source and a PIXcel 3D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96-well sample holder with mylar film and loaded into the instrument. The sample was scanned over the range of about 3° to about 40° 20 with a step size of 0.0131303° and 49 s per step. The XRPD diffractogram for Compound I Phosphate Salt Methanol Solvate is provided in FIG. 1 and the data is summarized below in Table 1.

TABLE 1
Peak list from XRPD diffractogram of Compound
I Phosphate Salt Methanol Solvate

| XRD Peaks | Angle (°2θ ± 0.2) | Intensity % |
|---|---|---|
| 1 | 15.8 | 100.0 |
| 2 | 20.7 | 89.2 |
| 3 | 12.7 | 59.5 |
| 4 | 8.5 | 54.2 |
| 5 | 19.5 | 45.5 |
| 6 | 18.7 | 36.8 |
| 7 | 13.9 | 35.6 |
| 8 | 10.2 | 30.3 |
| 9 | 22.5 | 29.5 |
| 10 | 21.5 | 27.4 |
| 11 | 3.9 | 26.4 |
| 12 | 20.0 | 24.9 |
| 13 | 19.2 | 24.5 |
| 14 | 24.9 | 24.0 |
| 15 | 19.6 | 23.3 |
| 16 | 21.8 | 21.5 |
| 17 | 27.4 | 21.3 |
| 18 | 12.9 | 21.0 |
| 19 | 25.2 | 20.8 |
| 20 | 14.8 | 20.7 |
| 21 | 17.3 | 18.0 |
| 22 | 9.6 | 17.8 |
| 23 | 20.4 | 17.0 |
| 24 | 17.6 | 15.9 |
| 25 | 16.0 | 15.5 |
| 26 | 11.4 | 13.9 |
| 27 | 18.4 | 13.7 |
| 28 | 25.5 | 12.5 |
| 29 | 27.9 | 12.2 |
| 30 | 27.6 | 11.1 |
| 31 | 12.5 | 10.8 |
| 32 | 23.5 | 10.7 |

### C. Solid-State NMR

The ¹³C CPMAS of Compound I Phosphate Salt Methanol Solvate (FIG. 2) was acquired at 275 K with 12.5 kHz spinning and using adamantane as a reference. The peaks are listed in Table 2 below.

TABLE 2
Peak list from ¹³C CPMAS of Compound
I Phosphate Salt Methanol Solvate

| Peak # | Chem Shift [ppm] | Intensity [rel] |
|---|---|---|
| 1 | 146.8 | 54.0 |
| 2 | 145.8 | 50.8 |
| 3 | 143.9 | 47.8 |
| 4 | 140.6 | 82.3 |
| 5 | 139.5 | 66.0 |
| 6 | 129.4 | 71.6 |
| 7 | 128.5 | 56.2 |
| 8 | 127.9 | 58.2 |
| 9 | 126.7 | 46.5 |
| 10 | 73.8 | 94.9 |
| 11 | 72.2 | 95.2 |
| 12 | 66.3 | 66.8 |
| 13 | 64.2 | 61.7 |
| 14 | 62.8 | 69.1 |
| 15 | 61.6 | 77.9 |
| 16 | 49.7 | 56.9 |
| 17 | 48.5 | 80.3 |
| 18 | 47.1 | 100.0 |
| 19 | 45.5 | 57.9 |
| 20 | 43.0 | 51.0 |
| 21 | 40.5 | 73.1 |
| 22 | 40.1 | 65.6 |
| 23 | 38.9 | 66.2 |
| 24 | 37.7 | 62.1 |
| 25 | 36.8 | 58.6 |
| 26 | 17.7 | 78.3 |
| 27 | 15.7 | 78.5 |

The ¹⁹F MAS of Compound I Phosphate Salt Methanol Solvate (FIG. 3) was acquired at 275 K with 12.5 kHz spinning and using adamantane as a reference, with ¹⁹F background subtracted. The peaks are listed in Table 3 below.

TABLE 3
Peak list from ¹⁹F MAS of Compound
I Phosphate Salt Methanol Solvate

| Peak # | Chem Shift [ppm] | Intensity [rel] |
|---|---|---|
| 1 | −54.7 | 11.8 |
| 2 | −57.7 | 12.5 |

The ³¹P CPMAS of Compound I Phosphate Salt Methanol Solvate (FIG. 4) was acquired at 275 K with 12.5 kHz spinning and using adamantane as a reference. The peaks are listed in Table 4 below.

TABLE 4
Peak list from ³¹P CPMAS of Compound
I Phosphate Salt Methanol Solvate

| Peak # | Chem Shift [ppm] | Intensity [rel] |
|---|---|---|
| 1 | 2.5 | 93.9 |
| 2 | 1.8 | 100.0 |

### D. Single Crystal Elucidation

Single crystals having the Compound I Phosphate Salt Methanol Solvate structure were grown from a 2-butanone (MEK), methanol, and water solution at room temperature (25±2° C.). X-ray diffraction data were acquired at 100 K on a Bruker diffractometer equipped with Cu K_{α} radiation (λ=1.54178 Å) and a CMOS detector. The structure was solved and refined using SHELX programs (Sheldrick, G. M., Acta Cryst., (2008) A64, 112-122). The peaks are summarized in Table 5 below.

TABLE 5
Single crystal elucidation of Compound
I Phosphate Salt Methanol Solvate

| Crystal System | Orthorhombic |
|---|---|
| Space Group | P2₁2₁2₁ |
| a (Å) | 9.3569(6) |
| b (Å) | 10.5460(6) |
| c (Å) | 44.580(3) |
| α (°) | 90 |
| β (°) | 90 |
| γ (°) | 90 |
| V (Å³) | 4399.0 |
| Z/Z′ | 4/2 |
| Temperature | 100 K |

2. Compound I Phosphate Salt Hydrate Form A

### A. Synthetic Procedure

Compound I Phosphate Salt Hydrate Form A was prepared by Method A or B described above.

Alternatively, 2.05 g Compound I Phosphate Salt Methanol Solvate was dried at 50° C. for 21 hours with N2 purge. The resultant solid was Compound I Phosphate Salt Hydrate Form A.

### B. X-Ray Powder Diffraction

The powder, X-ray powder diffraction (XRPD), diffractogram of Compound I Phosphate Salt Hydrate Form A (FIG. 5) was acquired at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder was placed in sample stage AP CHC stage and CHC chamber. The CHC chamber was attached to a water pump which collected the relative humidity. The relative humidity in the chamber was stepwise changed in increments, starting at 5% for 1 hour, then increased to 10% and held for an hour followed by 10% relative humidity (RH) stepwise increased to 60% and held for an hour at each, with a jump at 60% to 90% and held for 1 hour. The CHC Chamber was then held at 90% for an additional hour, then decreased from 90% to 80% and held for 3 hours, then from 80% to 70% and held for 3 hours, then from 70% to 60% and held for 3 hours, then from 60% to 10% decreased stepwise by 10% RH and held for an hour at each step and last decreased from 10% to 5% and held for an hour. At the hour time point, XRPD collection was run over the range of about 3° to about 40° 20 with a step size of 0.0131303° and 49 s per step.

Variable Humidity XRPD (VH-XRPD): Compound I Phosphate Salt Hydrate Form A was observed to have continuous peak shift which are all (within ±0.2° 20) from 5-90% relative humidity (FIG. 6, Table 6).

TABLE 6
Peak list from XRPD diffractogram of Compound I
Phosphate Salt Hydrate Form A

| | Relative Humidity | | Relative Humidity | | Relative Humidity | |
|---|---|---|---|---|---|---|
| | 40% | | 5% | | 90% | |
| XRD | Angle | Intensity | Angle | Intensity | Angle | Intensity |
| Peaks | (°2θ ± 0.2) | % | (°2θ ± 0.2) | % | (°2θ ± 0.2) | % |
| 1 | 19.9 | 100.0 | 19.9 | 100.0 | 19.9 | 100.0 |
| 2 | 8.6 | 76.2 | 8.6 | 79.2 | 8.6 | 65.3 |
| 3 | 28.3 | 64.3 | 28.3 | 69.4 | 28.3 | 60.9 |
| 4 | 20.4 | 56.7 | 20.4 | 61.9 | 20.4 | 55.2 |
| 5 | 21.0 | 43.0 | 22.8 | 37.1 | 21.0 | 48.7 |
| 6 | 22.8 | 41.4 | 17.2 | 31.9 | 27.8 | 44.9 |
| 7 | 17.2 | 38.3 | 21.9 | 30.1 | 22.8 | 40.9 |
| 8 | 27.8 | 37.2 | 21.1 | 29.7 | 17.2 | 40.5 |
| 9 | 26.4 | 28.4 | 27.0 | 29.3 | 19.5 | 30.9 |
| 10 | 17.8 | 27.2 | 15.7 | 23.7 | 25.5 | 30.6 |
| 11 | 15.7 | 26.8 | 27.8 | 22.9 | 17.8 | 29.2 |
| 12 | 25.5 | 26.2 | 25.8 | 18.2 | 15.8 | 26.6 |
| 13 | 19.5 | 25.8 | 16.9 | 17.6 | 21.9 | 25.8 |
| 14 | 21.9 | 25.5 | 17.8 | 17.1 | 16.9 | 24.1 |
| 15 | 27.1 | 23.3 | 19.6 | 16.7 | 27.1 | 23.2 |
| 16 | 16.9 | 22.7 | 26.4 | 15.9 | 26.4 | 22.5 |
| 17 | 21.7 | 20.1 | 25.1 | 15.2 | 25.1 | 20.0 |
| 18 | 25.1 | 19.4 | 25.4 | 15.1 | 25.9 | 16.1 |
| 19 | 25.9 | 16.6 | 22.1 | 14.3 | 25.3 | 15.1 |
| 20 | 19.7 | 14.7 | 17.7 | 14.2 | 13.0 | 13.7 |
| 21 | 22.0 | 13.6 | 12.9 | 12.6 | 20.6 | 13.4 |
| 22 | 13.0 | 13.1 | 18.5 | 12.2 | 18.5 | 12.1 |
| 23 | 25.3 | 12.7 | 27.4 | 11.6 | 11.5 | 10.4 |
| 24 | 18.5 | 12.3 | 11.5 | 11.5 | 17.6 | 10.4 |
| 25 | 17.6 | 11.9 | | | 27.4 | 10.4 |
| 26 | 11.5 | 11.5 | | | 13.1 | 10.2 |
| 27 | 27.4 | 11.0 | | | | |
| 28 | 13.2 | 10.1 | | | | |

### C. Solid-State NMR

The ¹³C CPMAS of Compound I Phosphate Salt Hydrate Form A (FIG. 7) was acquired at 275 K and 43% relative humidity (RH) with 12.5 kHz spinning and using adamantane as a reference. The peaks are listed in Table 7 below.

TABLE 7
Peak list from ¹³C CPMAS of Compound
I Phosphate Salt Hydrate Form A

| Peak # | Chem Shift [ppm] | Intensity [rel] |
|---|---|---|
| 1 | 146.3 | 42.1 |
| 2 | 145.8 | 45.6 |
| 3 | 144.0 | 42.5 |
| 4 | 141.7 | 58.3 |
| 5 | 139.3 | 51.8 |
| 6 | 129.4 | 46.4 |
| 7 | 128.6 | 52.3 |
| 8 | 126.6 | 46.8 |
| 9 | 73.6 | 87.5 |
| 10 | 73.2 | 83.2 |
| 11 | 66.1 | 38.9 |
| 12 | 64.3 | 43.7 |
| 13 | 62.7 | 55.1 |
| 14 | 62.1 | 62.3 |
| 15 | 48.9 | 44.6 |
| 16 | 47.3 | 70.8 |
| 17 | 45.4 | 50.6 |
| 18 | 43.0 | 39.6 |
| 19 | 41.6 | 48.8 |
| 20 | 38.4 | 100.0 |
| 21 | 36.7 | 48.3 |
| 22 | 16.0 | 94.4 |

The ¹⁹F MAS of Compound I Phosphate Salt Hydrate Form A (FIGS. 8, 9) was acquired at 275 K and 0%, 6%, 22%, 43%, 53%, 75%, and 100% relative humidity (RH) with 12.5 kHz spinning and using adamantane as a reference. The peaks are listed in Tables 8 and 9 below.

TABLE 8
Peak list from¹⁹F MAS of Compound I Phosphate
Salt Hydrate Form A at 43% RH

| Peak # | Chem Shift [ppm] | Intensity [rel] |
|---|---|---|
| 1 | −53.8 | 11.0 |
| 2 | −57.4 | 12.5 |

TABLE 9
Effect of relative humidity on ¹⁹F MAS
of Compound I Phosphate Salt Hydrate

| RH [%] | Peak 1 [ppm] | Peak 2 [ppm] |
|---|---|---|
| 0 | −53.4 | −57.6 |
| 6 | −53.6 | −57.5 |
| 22 | −53.8 | −57.5 |
| 33 | −53.8 | −57.4 |
| 43 | −53.8 | −57.4 |
| 53 | −53.8 | −57.4 |
| 75 | −53.9 | −57.4 |
| 100 | −53.9 | −57.4 |

The ³¹P CPMAS of Compound I Phosphate Salt Hydrate Form A (FIGS. 10, 11; Tables 10 and 11) was acquired at 275 K and 0%, 6%, 22%, 43%, 53%, 75%, and 100% relative humidity (RH) with 12.5 kHz spinning and using adamantane as a reference.

TABLE 10
Peak list from ³¹P CPMAS of Compound I
Phosphate Salt Hydrate Form A at 43% RH

| Peak # | Chem Shift [ppm] | Intensity [rel] |
|---|---|---|
| 1 | 4.2 | 46.4 |
| 2 | 2.6 | 100.0 |

TABLE 11
Effect of relative humidity on ³¹P CPMAS
of Compound I Phosphate Salt Hydrate

| RH [%] | Peak 1 [ppm] | Peak 2 [ppm] |
|---|---|---|
| 0 | 6.1 | 2.6 |
| 6 | 5.1 | 2.6 |
| 22 | 4.4 | 2.6 |
| 33 | 4.2 | 2.6 |
| 43 | 4.2 | 2.6 |
| 53 | 4.1 | 2.5 |
| 75 | 4.0 | 2.5 |
| 100 | 3.8 | 2.5 |

### D. Thermogravimetric Analysis

Thermogravimetric analysis (TGA) of Compound I Phosphate Salt Hydrate Form A was performed using the TA Instruments Q5000 TGA. A sample with a weight of approximately 1-10 mg was scanned from ambient to 300° C. at a heating rate of 10° C./min with nitrogen purge. The TGA thermogram shows around 0.5% weight loss from ambient temperature up until 150° C. (FIG. 12).

### E. Differential Scanning Calorimetry Analysis

Differential Scanning calorimetry (DSC) analysis of Compound I Phosphate Salt Hydrate Form A was performed using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heat rate at of 2° C. per min to a temperature to 300° C. The thermogram shows two endotherm peaks around 226° C. and 251° C. (FIG. 13).

### F. Single Crystal Elucidation

Single crystals having the Compound I Phosphate Salt Hydrate Form A structure were grown from an acetone solution at 40° C. X-ray diffraction data were acquired at 100 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122). The results are summarized in Table 12 below.

TABLE 12
Single crystal elucidation of Compound
I Phosphate Salt Hydrate Form A

| Crystal System | Orthorhombic |
|---|---|
| Space Group | P2₁2₁2₁ |
| a (Å) | 8.9452(2) |
| b (Å) | 10.4539(2) |
| c (Å) | 45.0276(10) |
| α (°) | 90 |
| β (°) | 90 |
| γ (°) | 90 |
| V (Å3) | 4210.63(16) |
| Z/Z′ | 4/2 |
| Temperature | 100 K |

Single crystals having the Compound I Phosphate Salt Hydrate Form A (dry) structure were obtained by drying crystals of Compound I Phosphate Salt Hydrate Form At 300 K under dry nitrogen for 1 hour. X-ray diffraction data were acquired at 100 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122). The results are summarized in Table 13 below.

TABLE 13
Single crystal elucidation of Compound
I Phosphate Salt Hydrate Form A (Dry)

| Crystal System | Orthorhombic |
|---|---|
| Space Group | P2₁2₁2₁ |
| a (Å) | 8.7937(4) |
| b (Å) | 10.4841(6) |
| c (Å) | 45.2323(16) |
| α (°) | 90 |
| β (°) | 90 |
| γ (°) | 90 |
| V (Å3) | 4170.1(3) |
| Z/Z′ | 4/2 |
| Temperature | 100 K |

3. Compound I Free Form Monohydrate

### A. Synthetic Procedure

Compound I free form Monohydrate may be prepared as described as above. Alternatively, Compound I free form Monohydrate may be prepared as follows. Amorphous Compound I (30 mg) was added to saline (1 mL). After mild vortexing to see if the material would dissolve, a white milky precipitate formed. The sample was left overnight at ambient temperature. The solid material was filtered using a 0.22 μm PVDF Eppendorf filter tube, rinsing with ice cold water. The sample was dried in a vacuum oven at 45° C. overnight. Both the wet cake and dried materials were crystalline Compound I free form Monohydrate.

### B. X-Ray Powder Diffraction

The powder, X-ray powder diffraction (XRPD), diffractogram of Compound I free form Monohydrate was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303 and 49 s per step. The XRPD diffractogram for Compound I free form Monohydrate is provided in FIG. 14 and the data is summarized below in Table 14.

TABLE 14
Peak list from XRPD diffractogram
of Compound I free form Monohydrate

| XRD Peaks | Angle (°2θ ± 0.2) | Intensity % |
|---|---|---|
| 1 | 16.7 | 100.0 |
| 2 | 21.7 | 37.0 |
| 3 | 8.7 | 23.3 |
| 4 | 12.8 | 18.4 |
| 5 | 19.8 | 15.9 |
| 6 | 25.8 | 15.8 |
| 7 | 13.8 | 15.1 |
| 8 | 15.5 | 12.7 |
| 9 | 24.3 | 12.7 |

### C. Solid-State NMR

The ¹³C CPMAS of Compound I free form Monohydrate (FIG. 15) was acquired at 275 K was acquired at 275 K and 43% relative humidity (RH) with 12.5 kHz spinning and using adamantane as a reference. The peaks are listed in Table 15 below. Additionally, the ¹³C CPMAS of Compound I free form Monohydrate following dehydration (80° C. in rotor overnight (2×), 80° C. weekend incubation with P₂O₅) (FIG. 16) was acquired at 275 K with 12.5 kHz spinning and using adamantane as a reference. The peaks are listed in Table 16 below.

TABLE 15
Peak list from ¹³C CPMAS of Compound I free form Monohydrate

| Peak # | Chem Shift [ppm] | Intensity [rel] |
|---|---|---|
| 1 | 149.6 | 57.4 |
| 2 | 149.4 | 33.3 |
| 3 | 135.3 | 63.4 |
| 4 | 129.6 | 28.0 |
| 5 | 127.7 | 23.3 |
| 6 | 126.2 | 26.9 |
| 7 | 74.4 | 100.0 |
| 8 | 68.1 | 40.7 |
| 9 | 61.6 | 47.1 |
| 10 | 49.8 | 47.2 |
| 11 | 47.8 | 33.0 |
| 12 | 47.0 | 36.0 |
| 13 | 39.3 | 41.7 |
| 14 | 35.1 | 43.2 |
| 15 | 24.9 | 55.8 |

TABLE 16
Peak list from ¹³C CPMAS of Dehydrated
Compound I free form Monohydrate

| Peak # | Chem Shift [ppm] | Intensity [rel] |
|---|---|---|
| 1 | 150.9 | 33.4 |
| 2 | 150.0 | 53.9 |
| 3 | 135.3 | 64.6 |
| 4 | 129.6 | 30.1 |
| 5 | 127.2 | 29.2 |
| 6 | 126.6 | 32.6 |
| 7 | 74.7 | 100.0 |
| 8 | 68.4 | 14.9 |
| 9 | 61.5 | 38.6 |
| 10 | 50.7 | 48.5 |
| 11 | 48.8 | 22.2 |
| 12 | 48.3 | 41.7 |
| 13 | 47.5 | 23.4 |
| 14 | 47.2 | 41.7 |
| 15 | 36.8 | 45.2 |
| 16 | 35.8 | 42.5 |
| 17 | 25.6 | 56.2 |

The ¹⁹F MAS of Compound I free form Monohydrate (FIG. 17) was acquired at 275 K and 43% relative humidity (RH) with 12.5 kHz spinning and using adamantane as a reference, with ¹⁹F background subtracted. The peaks are listed in Table 17 below. Additionally, the ¹⁹F MAS of Compound I free form Monohydrate following dehydration (80° C. in rotor overnight (2×), 80° C. weekend incubation with P₂O₅) (FIG. 18) was acquired at 275 K with 12.5 kHz spinning and using adamantane as a reference, with ¹⁹F background subtracted. The peaks are listed in Table 18 below.

TABLE 17
Peak list from ¹⁹F MAS of Compound I free form Monohydrate

| Peak # | Chem Shift [ppm] | Intensity [rel] |
|---|---|---|
| 1 | −55.8 | 12.5 |

TABLE 18
Peak list from ¹⁹F MAS of Dehydrated
Compound I free form Monohydrate

| Peak # | Chem Shift [ppm] | Intensity [rel] |
|---|---|---|
| 1 | −55.5 | 12.5 |

### D. Thermogravimetric Analysis

Thermal gravimetric analysis of Compound I free form Monohydrate was performed using the TA5500 Discovery TGA. A sample with a weight of approximately 1-10 mg was scanned from ambient temperature to 250° C. at a heating rate of 10° C./min with nitrogen purge. The TGA thermogram showed around ˜3-4% weight loss from ambient temperature up until 100° C. (FIG. 19).

### E. Differential Scanning Calorimetry Analysis

The Differential Scanning calorimetric analysis of Compound I free form Monohydrate was performed using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heated at a rate of 2° C. per min to a temperature to 300° C. The thermogram showed three endotherm peaks around 61° C., 94° C., and 111° C. (FIG. 20).

### F. Single Crystal Elucidation

Single crystals having the Compound I free form Monohydrate structure were crystallized from saline. X-ray diffraction data were acquired at 100 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122). The results are summarized in Table 19 below.

TABLE 19
Single crystal elucidation of Compound I free form Monohydrate

| Crystal System | Tetragonal |
|---|---|
| Space Group | P4₃ |
| a (Å) | 14.2402(4) |
| b (Å) | 14.2402(4) |
| c (Å) | 9.3198(4) |
| α (°) | 90 |
| β (°) | 90 |
| γ (°) | 90 |
| V (Å³) | 1889.90(13) |
| Z/Z′ | 4/1 |
| Temperature | 100 K |

Single crystals having the Compound I free form structure were obtained by drying a single crystal of Compound I free form Monohydrate at 325 K for 1 hour under dry nitrogen. X-ray diffraction data were acquired at 100 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122). The results are summarized in Table 20 below.

TABLE 20
Single crystal elucidation of Compound I Free Form Monohydrate

| Crystal System | Tetragonal |
|---|---|
| Space Group | P4₃ |
| a (Å) | 14.2909(4) |
| b (Å) | 14.2909(4) |
| c (Å) | 9.1637(4) |
| α (°) | 90 |
| β (°) | 90 |
| γ (°) | 90 |
| V (Å³) | 1871.50(13) |
| Z/Z′ | 4/1 |
| Temperature | 100 K |

4. Compound I Phosphate Salt MEK Solvate

### A. Synthetic Procedure

Compound I Phosphate Salt Hydrate Form A (25 mg) was added to 2-butanone (MEK) (1 mL) in an HPLC vial. The sample was mixed and formed a slurry. The slurry was placed in a cold room at 5° C. with a small stir bar for 11 days. The solid material was centrifuged and filtered using a 0.22 μm PVDF Eppendorf filter tube at room temperature. The XRD of the wet cake sample showed that it was Compound I Phosphate Salt MEK Solvate.

### B. X-Ray Powder Diffraction

The powder, X-ray powder diffraction (XRPD), diffractogram of Compound I Phosphate Salt MEK Solvate was acquired at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film as well Kapton tape over the sample and loaded into the instrument. The sample was scanned over the range of about 3° to about 40° 20 with a step size of 0.0131303° and 49 s per step. The XRPD diffractogram for Compound I Phosphate Salt MEK Solvate is provided in FIG. 21 and the data is summarized below in Table 21.

TABLE 21
Peak list from XRPD diffractogram of
Compound I Phosphate Salt MEK Solvate

| XRD Peaks | Angle (°2θ ± 0.2) | Intensity % |
|---|---|---|
| 1 | 20.1 | 100.0 |
| 2 | 15.4 | 85.7 |
| 3 | 8.6 | 80.8 |
| 4 | 15.7 | 36.5 |
| 5 | 19.4 | 32.1 |
| 6 | 18.2 | 32.0 |
| 7 | 21.7 | 30.8 |
| 8 | 21.9 | 29.0 |
| 9 | 13.2 | 28.6 |
| 10 | 23.8 | 25.9 |
| 11 | 10.8 | 25.1 |
| 12 | 10.5 | 24.1 |
| 13 | 21.0 | 23.0 |
| 14 | 22.8 | 21.7 |
| 15 | 17.5 | 18.8 |
| 16 | 18.4 | 18.2 |
| 17 | 26.7 | 16.8 |
| 18 | 22.4 | 14.4 |
| 19 | 3.8 | 12.4 |
| 20 | 8.3 | 11.0 |
| 21 | 16.5 | 10.6 |

### C. Solid-State NMR

The ¹³C CPMAS of Compound I Phosphate Salt MEK Solvate (FIG. 22) was acquired at 275 K with 12.5 kHz spinning and using adamantane as a reference. The peaks are listed in Table 22 below.

TABLE 22
Peak list from ¹³C CPMAS of Compound
I Phosphate Salt MEK Solvate

| Peak # | Chem Shift [ppm] | Intensity [rel] |
|---|---|---|
| 1 | 146.6 | 36.9 |
| 2 | 145.8 | 41.2 |
| 3 | 144.1 | 34.3 |
| 4 | 143.6 | 32.3 |
| 5 | 142.0 | 55.2 |
| 6 | 140.9 | 20.2 |
| 7 | 139.4 | 41.6 |
| 8 | 138.4 | 26.0 |
| 9 | 130.7 | 16.3 |
| 10 | 129.6 | 52.8 |
| 11 | 128.7 | 46.9 |
| 12 | 128.0 | 32.6 |
| 13 | 126.5 | 54.5 |
| 14 | 73.7 | 79.9 |
| 15 | 73.2 | 86.8 |
| 16 | 66.3 | 60.6 |
| 17 | 64.3 | 35.0 |
| 18 | 63.3 | 25.6 |
| 19 | 62.7 | 48.2 |
| 20 | 62.3 | 73.8 |
| 21 | 50.4 | 31.2 |
| 22 | 48.8 | 54.4 |
| 23 | 48.4 | 32.8 |
| 24 | 47.4 | 57.8 |
| 25 | 46.3 | 24.0 |
| 26 | 45.5 | 42.2 |
| 27 | 44.3 | 23.2 |
| 28 | 43.3 | 31.7 |
| 29 | 42.3 | 28.9 |
| 30 | 41.9 | 40.4 |
| 31 | 38.4 | 100.0 |
| 32 | 37.5 | 56.0 |
| 33 | 36.8 | 48.4 |
| 34 | 35.3 | 23.6 |
| 35 | 29.5 | 24.2 |
| 36 | 16.0 | 74.8 |
| 37 | 15.2 | 33.7 |
| 38 | 7.4 | 44.5 |

The ¹⁹F MAS of Compound I Phosphate Salt MEK Solvate (FIG. 23) was acquired at 275 K with 12.5 kHz spinning and using adamantane as a reference, with ¹⁹F background subtracted. The peaks are listed in Table 23 below.

TABLE 23
Peak list from ¹⁹F MAS of Compound
I Phosphate Salt MEK Solvate

| Peak # | Chem Shift [ppm] | Intensity [rel] |
|---|---|---|
| 1 | −53.6 | 10.0 |
| 2 | −55.2 | 5.2 |
| 3 | −57.2 | 12.5 |

The ³¹P CPMAS of Compound I Phosphate Salt MEK Solvate was acquired at 275 K with 12.5 kHz spinning and using adamantane as a reference. The peaks are listed in Table 24 below.

TABLE 24
Peak list from ³¹P CPMAS of Compound
I Phosphate Salt MEK Solvate

| Peak # | Chem Shift [ppm] | Intensity [rel] |
|---|---|---|
| 1 | 4.8 | 94.7 |
| 2 | 2.7 | 15.7 |
| 3 | 0.1 | 100.0 |

5. Compound I Maleate Form A (Salt or Co-Crystal)

### A. Synthetic Procedure

˜105 mg of Compound I free form Monohydrate was dissolved in 7 ml of acetonitrile. ˜31.5 mg of maleic acid was added to same solution, a suspension was observed and stirred at ambient temperature for 3 days. Centrifuged the suspension and air dried the wet cake. Took the solids at on TGA and heated the solids to 165° C. and ran XRPD confirmed Compound I Maleate Form A.

### B. X-Ray Powder Diffraction

The powder, X-ray powder diffraction (XRPD), diffractogram of Compound I Maleate Salt Form A was acquired at room temperature in reflection mode using a PANalytical X'Pert Powder system (Malvern PANalytical Inc, Westborough, Mass.). The X-Ray generator operated at a voltage of 45 kV and a current of 40 mA with copper radiation (1.54060 Å). The powder sample was placed on a Si zero-background holder and loaded into the instrument. The sample was scanned with divergence slit fixed ⅛° and scan mode was continuous over the range of about 3° to about 40° 20 with a step size of 0.0131° and 18.87 s per scan step.

The XRPD diffractogram for Compound I Maleate Salt Form A is provided in FIG. 39 and the data is summarized in Table 25.

TABLE 25
Peak list from XRPD diffractogram
of Compound I Maleate Salt Form A

| No. | Pos. [±0.2, °2θ] | Rel. Int. [%] |
|---|---|---|
| *1 | 18.3 | 100.0 |
| 2 | 13.7 | 70.9 |
| 3 | 14.5 | 30.5 |
| 4 | 27.6 | 20.2 |
| *5 | 20.0 | 15.3 |
| 6 | 15.5 | 10.3 |

### C. Thermogravimetric Analysis

Thermal gravimetric analysis of Compound I Maleate Form A was measured using the TA Instruments 550 TGA. A sample with weight of approximately 1-10 mg in a open platinum pan. The program was set to heat from ambient to at a heating rate of 10° C. per min to 300° C. with nitrogen purge. The TGA thermogram shows minimal weight loss until degradation (FIG. 40).

### D. Differential Scanning Calorimetry Analysis

DSC analysis of Compound I Maleate Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to heat from ambient to at a heating rate of 10° C. per min to 300° C. The thermogram shows one endotherm peak at 201° C. (FIG. 41).

6. Compound I Maleate Form B (Salt or Co-Crystal)

### A. Synthetic Procedure

˜105 mg of Compound I free form Monohydrate was dissolved in 7 ml of ethanol. ˜31.5 mg of maleic acid was added to same solution, which was then stirred at ambient temperature for 3 days. The solution was then fast evaporated for 5 days and solids where observed. Took the solids at on TGA and heated the solids to 150° C. and ran XRPD confirmed Compound I Maleate Form B

### B. X-Ray Powder Diffraction

The powder, X-ray powder diffraction (XRPD), diffractogram of Compound I Maleate Form B was acquired at room temperature in reflection mode using a PANalytical X'Pert Powder system (Malvern PANalytical Inc, Westborough, Mass.). The X-Ray generator operated at a voltage of 45 kV and a current of 40 mA with copper radiation (1.54060 Å). The powder sample was placed on a Si zero-background holder and loaded into the instrument. The sample was scanned with divergence slit fixed ⅛° and scan mode was continuous over the range of about 3° to about 40° 20 with a step size of 0.0131° and 18.87 s per scan step.

The XRPD diffractogram for Compound I Maleate Salt Form B is provided in FIG. 42 and the data is summarized in Table 26.

TABLE 26
Peak list from XRPD diffractogram
of Compound I Maleate Salt Form B

| No. | Pos. [±0.2, °2θ] | Rel. Int. [%] |
|---|---|---|
| 1 | 18.3 | 100.0 |
| 2 | 15.4 | 34.3 |
| 3 | 19.6 | 34.3 |
| 4 | 13.8 | 12.0 |
| 5 | 14.7 | 10.5 |
| 6 | 26.0 | 10.0 |
| *7 | 4.9 | 10.0 |

### C. Thermogravimetric Analysis

Thermal gravimetric analysis of Compound I Maleate Form B was measured using the TA Instruments 550 TGA. A sample with weight of approximately 1-10 mg in an open platinum pan. The program was set to heat from ambient to at a heating rate of 10° C. per min to 300° C. with nitrogen purge. The TGA thermogram shows minimal weight loss until degradation (FIG. 43).

### D. Differential Scanning Calorimetry Analysis

DSC analysis of Compound I Maleate Form B was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to heat from ambient to at a heating rate of 10° C. per min to 300° C. The thermogram shows one endotherm peak 206° C. (FIG. 44).

7. Compound I Fumaric Acid Form a (Salt or Co-Crystal)

### A. Synthetic Procedure

In a high through-put ball-mill added 3:4 molar ratio of Compound I monohydrate and fumaric acid (˜75 mg to ˜30 mg) to 2 ml vial with 2.8 mm ceramic (zirconium oxide) beads and 15 ul of water. The vail was placed in the high through-put ball mill ran at 7500 RPM for 60 seconds with 10 second pause for 3 cycles. The solid was then analyzed by XRPD, then placed in vacuum oven at ° C. overnight and again analyzed by XRPD and confirmed to be Compound I Fumaric Acid Form A.

### B. X-Ray Powder Diffraction

The powder, X-ray powder diffraction (XRPD), diffractogram of Compound I Fumaric Acid Form A was acquired at room temperature in transmission mode using a PANalytical Empyrean system equipped with a sealed tube source and a PIXcel 3D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound I Fumaric Acid Form A is provided in FIG. 45 and the data is summarized in Table 27.

TABLE 27
Peak list from XRPD diffractogram
of Compound I Fumaric Acid Form A

| No. | Pos. [±0.2, °2θ] | Rel. Int. [%] |
|---|---|---|
| 1 | 21.5 | 100.0 |
| 2 | 14.4 | 44.6 |
| 3 | 14.6 | 32.2 |
| 4 | 16.9 | 24.7 |
| 5 | 20.9 | 23.7 |
| 6 | 20.7 | 23.4 |
| 7 | 17.5 | 19.9 |
| 8 | 9.5 | 18.0 |
| 9 | 19.7 | 16.7 |
| 10 | 28.3 | 15.5 |
| 11 | 21.0 | 15.4 |
| 12 | 19.1 | 14.8 |
| 13 | 15.6 | 13.9 |
| 14 | 19.5 | 13.9 |
| 15 | 23.2 | 12.8 |
| 16 | 22.5 | 12.7 |
| 17 | 25.7 | 11.3 |
| 18 | 17.3 | 10.9 |
| 19 | 29.4 | 10.2 |

### C. Solid-State NMR

The ¹³C CPMAS of Compound I Fumaric Acid Form A (FIG. 46) was acquired at 275 K with 12.5 kHz spinning and using adamantane as a reference. The peaks are listed in Table 28 below.

TABLE 28
Peak list from ¹³C CPMAS of Compound I Fumaric Acid Form A

| Peak # | Chem Shift [ppm] | Intensity [rel] |
|---|---|---|
| 1 | 172.4 | 8.3 |
| 2 | 171.4 | 7.3 |
| 3 | 148.4 | 5.6 |
| 4 | 143.8 | 6.1 |
| 5 | 142.1 | 5.7 |
| 6 | 135.5 | 7.7 |
| 7 | 130.7 | 7.2 |
| 8 | 128.1 | 8.3 |
| 9 | 127.3 | 5.8 |
| 10 | 124.3 | 0.9 |
| 11 | 121.5 | 0.9 |
| 12 | 72.9 | 10.0 |
| 13 | 65.7 | 6.6 |
| 14 | 61.8 | 7.8 |
| 15 | 50.8 | 7.2 |
| 16 | 48.3 | 6.5 |
| 17 | 47.3 | 0.5 |
| 18 | 42.0 | 5.9 |
| 19 | 38.3 | 7.5 |
| 20 | 34.6 | 5.3 |
| 21 | 17.2 | 8.7 |

The ¹⁹F MAS of Compound I Fumaric Acid Form A (FIG. 47) was acquired at 275 K with 12.5 kHz spinning and using adamantane as a reference, with ¹⁹F background subtracted. The peaks are listed in Table 29 below.

TABLE 29
Peak list from ¹⁹F MAS of Compound I Fumaric Acid Form A

| Peak # | Chem Shift [ppm] | Intensity [rel] |
|---|---|---|
| 1 | −55.8 | 10.0 |

### D. Thermogravimetric Analysis

Thermal gravimetric analysis of Compound I Fumaric Acid Form A was measured using the TA5500 Discovery TGA. A sample with weight of approximately 1-10 mg was scanned from ambient to 250° C. at a heating rate of 10° C./min with nitrogen purge. The TGA thermogram shows minimal weight loss from ambient temperature up until 100° C. (FIG. 48).

### E. Differential Scanning Calorimetry Analysis

The Differential Scanning calorimetric analysis of Compound I Fumaric Acid Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heat rate at of 2° C. per min to a temperature to 300° C. The thermogram shows two endotherm peaks at 137° C. and 165° C. (FIG. 49).

8. Compound I Free Form Form B

### A. Synthetic Procedure

˜200 mg of Compound I Monohydrate was heated to 120° C. in oven for 2 hours and then cooled to 90° C. with amorphous material in the oven and keep oven at 90° C. for 5 days. Took solids and analyzed by XRD and Compound I free form Form B was obtained.

˜10 mg of Compound I Amorphous was placed in a heptane vapor chamber for 7 days. Took solids and analyzed by XRD and Compound I free form Form B was obtained.

### B. X-Ray Powder Diffraction

The powder, X-ray powder diffraction (XRPD), diffractogram of Compound I free form Form B was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound I free form Form B is provided in FIG. 50 and the data is summarized in Table 30.

TABLE 30
Peak list from XRPD diffractogram
of Compound I free form Form B

| No. | Pos. [±0.2, °2θ] | Rel. Int. [%] |
|---|---|---|
| *1 | 13.1 | 100.0 |
| *2 | 20.6 | 76.1 |
| *3 | 17.5 | 47.2 |
| 4 | 21.6 | 34.5 |
| *5 | 15.8 | 33.6 |
| 6 | 26.9 | 33.4 |
| 7 | 23.6 | 28.6 |
| *8 | 18.9 | 27.5 |
| *9 | 13.9 | 25.0 |
| 10 | 19.1 | 24.5 |
| 11 | 11.7 | 18.0 |
| 12 | 14.2 | 14.0 |
| 13 | 22.1 | 13.3 |
| 14 | 24.6 | 11.8 |
| 15 | 20.1 | 11.1 |
| 16 | 9.2 | 10.9 |

### C. Solid-State NMR

The ¹³C CPMAS of Compound I free form Form B (FIG. 51) was acquired at 275 K with 12.5 kHz spinning and using adamantane as a reference. The peaks are listed in Table 31 below.

TABLE 31
Peak list from ¹³C CPMAS of Compound I free form Form B

| Peak # | Chem Shift [ppm] | Intensity [rel] |
|---|---|---|
| 1 | 152.2 | 3.9 |
| 2 | 148.1 | 5.0 |
| 3 | 140.0 | 6.7 |
| 4 | 130.4 | 4.0 |
| 5 | 128.7 | 2.2 |
| 6 | 125.1 | 3.7 |
| 7 | 73.7 | 10.0 |
| 8 | 63.4 | 6.2 |
| 9 | 62.5 | 7.6 |
| 10 | 47.9 | 7.6 |
| 11 | 45.9 | 5.7 |
| 12 | 43.1 | 4.2 |
| 13 | 35.7 | 4.5 |
| 14 | 23.5 | 8.2 |

The ¹⁹F MAS of Compound I free form Form B (FIG. 52) was acquired at 275 K and 43% relative humidity (RH) with 12.5 kHz spinning and using adamantane as a reference, with ¹⁹F background subtracted. The peaks are listed in Table 32 below.

TABLE 32
Peak list from ¹⁹F MAS of Compound I free form Form B

| Peak # | Chem Shift [ppm] | Intensity [rel] |
|---|---|---|
| 1 | −54.8 | 10.0 |

### D. Thermogravimetric Analysis

Thermal gravimetric analysis of Compound I free form Form B was measured using the TA5500 Discovery TGA. A sample with weight of approximately 1-10 mg was scanned from ambient to 250° C. at a heating rate of 10° C./min with nitrogen purge. The TGA thermogram shows minimal weight loss from ambient temperature up until 180° C. (FIG. 53).

### E. Differential Scanning Calorimetry Analysis

The Differential Scanning calorimetric analysis of Compound I free form Form B was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heat rate at of 2° C. per min to a temperature to 275° C. The thermogram shows two endotherm peaks at 132° C. (FIG. 54).

### F. Single Crystal Elucidation

Single crystals having the Compound I free form Form B structure were crystallized from Dry 90° C. Oven. X-ray diffraction data was acquired at 100 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122) and results are summarized in Table 33 below.

TABLE 33
Single crystal elucidation of Compound
I free form Form B acquired at 100 K

| Crystal System | Orthorhombic |
|---|---|
| Space Group | P2₁2₁2₁ |
| a (Å) | 8.1026(2) |
| b (Å) | 11.8447(3) |
| c (Å) | 18.9425(5) |
| α (°) | 90 |
| β (°) | 90 |
| γ (°) | 90 |
| V (Å³) | 1817.97(8) |
| Z/Z′ | 4/1 |
| Temperature | 100 K |

Single crystals having the Compound I free form Form B structure were crystallized from Dry 90° C. Oven. X-ray diffraction data was acquired at 298 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122) and results are summarized in Table 34 below.

TABLE 34
Single crystal elucidation of Compound
I free form Form B acquired at 298 K

| Crystal System | Orthorhombic |
|---|---|
| Space Group | P2₁2₁2₁ |
| a (Å) | 8.21130(10) |
| b (Å) | 11.9417(2) |
| c (Å) | 19.0812(3) |
| α (°) | 90 |
| β (°) | 90 |
| γ (°) | 90 |
| V (Å³) | 1871.04(5) |
| Z/Z′ | 4/1 |
| Temperature | 298 K |

9. Compound I Free Form Form C

### A. Synthetic Procedure

Seed of Compound I free form Form C was obtained by thermal treatment on a physical mixture of ˜100 mg Compound I monohydrate and ˜10 mg Compound II free form Form C in a TGA pan. The thermal treatment was conducted with TGA ramping at 10° C. per min to 120° C., isothermal at 120° C. for 60 minutes, then cooling at 2° C. per min down to 25° C. The seed produced with this thermal treatment was then added into a Compound I monohydrate heptane slurry. The slurry was kept at 50° C. for 7 days. The solids were isolated for XRPD and ssNMR analysis and confirmed to be pure Compound I free form Form C.

4 g of Compound I monohydrate was charged to a reactor followed by an 85% by volume mixture of heptane in ethyl acetate. The slurry was heated to 65° C. while agitating which remained a slurry at 65° C. To this slurry added Compound I free form Form C seeds (1 w %) and continued the agitation at 65° C. for at least 72 hours to achieve complete form conversion. The slurry was then hot filtered, and the wet solids were dried under vacuum at 50° C. with a nitrogen bleed to yield 3.72 g the solids were analyzed by XRPD for Compound I free form Form C.

### B. X-Ray Powder Diffraction

The X-ray powder diffraction (XRPD), diffractogram of Compound I free form Form C was acquired at room temperature in transmission mode using a PANalytical Empyrean system equipped with a sealed tube source and a PIXcel 1D Medipix-3 detector (Malvern PANalytical Inc, Westborough, Mass.). The X-ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound I free form Form C is provided in FIG. 55 and the data is summarized in Table 35.

TABLE 35
Peak list from XRPD diffractogram
of Compound I free form Form C

| No. | Pos. [±0.2, °2θ] | Rel. Int. [%] |
|---|---|---|
| *1 | 21.0 | 100.0 |
| 2 | 17.7 | 67.5 |
| 3 | 12.9 | 49.6 |
| 4 | 18.6 | 45.6 |
| *5 | 15.4 | 24.6 |
| *6 | 11.1 | 22.7 |
| 7 | 22.5 | 21.6 |
| *8 | 19.8 | 21.0 |
| 9 | 20.8 | 20.8 |
| 10 | 25.7 | 15.8 |
| *11 | 9.5 | 14.4 |
| *12 | 14.7 | 13.9 |
| 13 | 17.4 | 12.6 |
| 14 | 25.9 | 12.4 |
| 15 | 23.3 | 10.8 |
| 16 | 29.9 | 10.8 |

### C. Solid-State NMR

The ¹³C CPMAS of Compound I free form Form C (FIG. 56) was acquired at 275 K with 12.5 kHz spinning and using adamantane as a reference. The peaks are listed in Table 36 below.

TABLE 36
Peak list from ¹³C CPMAS of Compound I free form Form C

| Peak # | Chem Shift [ppm] | Intensity [rel] |
|---|---|---|
| 1 | 149.6 | 5.4 |
| 2 | 149.2 | 3.9 |
| 3 | 137.1 | 6.2 |
| 4 | 130.1 | 4.8 |
| 5 | 128.6 | 2.4 |
| 6 | 124.2 | 3.5 |
| 7 | 74.5 | 9.8 |
| 8 | 66.7 | 5.2 |
| 9 | 62.4 | 7.0 |
| 10 | 49.9 | 4.6 |
| 11 | 48.3 | 10.0 |
| 12 | 37.4 | 6.5 |
| 13 | 24.6 | 7.2 |

The ¹⁹F MAS of Compound I free form Form C (FIG. 57) was acquired at 275 K and 43% relative humidity (RH) with 12.5 kHz spinning and using adamantane as a reference, with ¹⁹F background subtracted. The peaks are listed in Table 37 below.

TABLE 37
Peak list from ¹⁹F MAS of Compound I free form Form C

| Peak # | Chem Shift [ppm] | Intensity [rel] |
|---|---|---|
| 1 | −54.0 | 10.0 |

### D. Thermogravimetric Analysis

Thermal gravimetric analysis of Compound I free form Form C was measured using the TA5500 Discovery TGA. A sample with weight of approximately 1-10 mg was scanned from ambient to 250° C. at a heating rate of 10° C./min with nitrogen purge. The TGA thermogram shows minimal weight loss from ambient temperature up until ˜190° C. (FIG. 58).

### E. Differential Scanning Calorimetry Analysis

The Differential Scanning calorimetric analysis of Compound I free form Form C was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heat rate at of 2° C. per min to a temperature to 200° C. The thermogram shows one endotherm peak at 134° C. (FIG. 59).

### F. Single Crystal Elucidation

Single crystals having the Compound I free form Form C structure were grown from heptane. X-ray diffraction data was acquired at 100 K on a Bruker diffractometer equipped with Cu Kα radiation (λ=1.54178 Å) and a CPAD detector. The structure was solved and refined using SHELX programs (Sheldrick, G. M., Acta Cryst., (2008) A64, 112-122) and results are summarized in Table 38 below.

TABLE 38
Single crystal elucidation of Compound I free form Form C

| Crystal System | Orthorhombic |
|---|---|
| Space Group | P2₁2₁2₁ |
| a (Å) | 10.1344(3) |
| b (Å) | 12.5319(5) |
| c (Å) | 13.4105(5) |
| α (°) | 90 |
| β (°) | 90 |
| γ (°) | 90 |
| V (Å³) | 1703.18(11) |
| Z/Z′ | 4/1 |
| Temperature | 100 K |

10. Compound I Phosphate Salt Form B

A. Synthetic Procedure

˜20 mg of Compound I Phosphate Hydrate Form A was added to 300 μl of 1-pentanol at 21-23° C. where a slurry was obtained mixing at 800 RPM for two weeks. The slurry was centrifuged and vacuum dried to form a wet cake at 40° C. for 7 days. The vacuum dried solids were characterized by XPRD, which confirmed Compound I Phosphate Salt Form B.

B. X-Ray Powder Diffraction

The X-ray powder diffractogram of Compound I Phosphate Salt Form B was acquired at room temperature in transmission mode using a PANalytical Empyrean system equipped with a sealed tube source and a PIXcel 3D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step (FIG. 98).

TABLE 80
Peak list from XRPD diffractogram
of Compound I Phosphate Salt Form B

| No. | Pos. [±0.2, °2θ] | Rel. Int. [%] |
|---|---|---|
| 1 | 9.7 | 100.0 |
| 2 | 20.9 | 91.2 |
| 3 | 22.8 | 60.4 |
| 4 | 13.9 | 60.1 |
| 5 | 6.9 | 54.1 |
| 6 | 9.0 | 52.0 |
| 7 | 17.3 | 48.8 |
| 8 | 16.6 | 41.5 |
| 9 | 10.7 | 28.9 |
| 10 | 11.4 | 27.7 |
| 11 | 21.4 | 26.0 |
| 12 | 19.7 | 25.4 |
| 13 | 23.3 | 24.7 |
| 14 | 24.9 | 23.6 |
| 15 | 23.1 | 23.3 |
| 16 | 17.0 | 22.4 |
| 17 | 20.2 | 21.4 |
| 18 | 22.3 | 18.8 |
| 19 | 21.5 | 18.8 |
| 20 | 28.0 | 17.5 |
| 21 | 12.3 | 16.3 |
| 22 | 22.1 | 14.1 |
| 23 | 26.6 | 11.3 |
| 24 | 23.6 | 11.2 |
| 25 | 14.2 | 10.0 |

C. Solid-State NMR

The ¹³C CPMAS of Compound I Phosphate Salt Form B (FIG. 101) was acquired at 275 K with 12.5 kHz spinning and using adamantane as a reference. The peaks are listed in Table 81 below.

TABLE 81
Peak list from ¹³C CPMAS of Compound I

| Peak # | Chem Shift [ppm] | Intensity [rel] |
|---|---|---|
| 1 | 147.7 | 46.7 |
| 2 | 145.6 | 49.1 |
| 3 | 144.2 | 43.6 |
| 4 | 143.4 | 44.2 |
| 5 | 142.1 | 48.5 |
| 6 | 135.3 | 65.4 |
| 7 | 130.1 | 24.9 |
| 8 | 128.9 | 29.1 |
| 9 | 128.0 | 78.9 |
| 10 | 126.3 | 40.0 |
| 11 | 124.0 | 9.4 |
| 12 | 121.2 | 8.7 |
| 13 | 74.2 | 100.0 |
| 14 | 73.5 | 92.0 |
| 15 | 66.2 | 43.4 |
| 16 | 63.6 | 50.4 |
| 17 | 61.3 | 40.7 |
| 18 | 50.5 | 51.8 |
| 19 | 49.7 | 62.2 |
| 20 | 48.2 | 49.3 |
| 21 | 46.2 | 46.0 |
| 22 | 44.2 | 38.3 |
| 23 | 42.9 | 25.0 |
| 24 | 38.8 | 49.4 |
| 25 | 38.5 | 61.4 |
| 26 | 37.4 | 66.0 |
| 27 | 36.3 | 33.2 |
| 28 | 18.0 | 71.2 |
| 29 | 16.5 | 80.8 |

The ¹⁹F MAS of Compound I Phosphate Salt Form B (FIG. 102) was acquired at 275 K with 12.5 kHz spinning and using adamantane as a reference. The peaks are listed in Table 82 below. Additionally, the ³¹P CPMAS of Compound I Phosphate Salt Form B (FIG. 103) was acquired at 275 K with 12.5 kHz spinning and using adamantane as a reference. The peaks are listed in Table 83 below.

TABLE 82
Peak list from 19F MAS of Compound I Phosphate Salt Form B

| Peak # | Chem Shift [ppm] | Intensity [rel] |
|---|---|---|
| 1 | −55.2 | 12.5 |

TABLE 83
Peak list from 31P CPMAS of Compound I Phosphate Salt Form B

| Peak # | Chem Shift [ppm] | Intensity [rel] |
|---|---|---|
| 1 | 6.1 | 96.2 |
| 2 | 4.5 | 100.0 |

D. Thermogravimetric Analysis

Thermal gravimetric analysis of Compound I Phosphate Salt Form B was measured using the TA5500 Discovery TGA. A sample with weight of approximately 1-10 mg was scanned from ambient to 300° C. at a heating rate of 10° C./min with nitrogen purge. The TGA thermogram shows minimal weight loss from ambient temperature up until 210° C. (FIG. 99).

E. Differential Scanning Calorimetry Analysis

DSC analysis of Compound I Phosphate Salt Form B was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heat rate at of 2° C. per min to a temperature to 300° C. The thermogram shows two endotherm peaks at 218° C. and 235° C. (FIG. 100).

11. Compound I Phosphate Salt Form C

A. Synthetic Procedure

˜20 mg of Compound I Phosphate Hydrate Form A was slurried in 300 ul of 1,4-dioxane at 21-23° C. for 2 weeks. The slurry was centrifuged and vacuum dried to form a wet cake at 40° C. for 7 days. The vacuum dried solids were characterized by XRPD, which confirmed Compound I Phosphate Salt Form C.

B. X-Ray Powder Diffraction

The X-ray powder diffractogram of Compound I Phosphate Salt Form C was acquired at room temperature in transmission mode using a PANalytical Empyrean system equipped with a sealed tube source and a PIXcel 3D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step (FIG. 104).

TABLE 84
Peak list from XRPD diffractogram
of Compound I Phosphate Salt Form C

| No. | Pos. [±0.2, °2θ] | Rel. Int. [%] |
|---|---|---|
| 1 | 14.5 | 100.0 |
| 2 | 10.4 | 79.6 |
| 3 | 12.4 | 66.1 |
| 4 | 18.7 | 63.6 |
| 5 | 11.6 | 56.3 |
| 6 | 18.3 | 48.8 |
| 7 | 25.0 | 47.4 |
| 8 | 20.7 | 46.3 |
| 9 | 11.4 | 45.8 |
| 10 | 18.9 | 41.0 |
| 11 | 25.9 | 37.5 |
| 12 | 5.8 | 26.5 |
| 13 | 19.8 | 25.9 |
| 14 | 16.0 | 24.5 |
| 15 | 15.2 | 24.0 |
| 16 | 15.7 | 21.6 |
| 17 | 8.2 | 20.2 |
| 18 | 19.6 | 20.1 |
| 19 | 20.0 | 19.0 |
| 20 | 20.5 | 18.5 |
| 21 | 14.9 | 18.5 |
| 22 | 24.6 | 17.4 |
| 23 | 22.8 | 17.0 |
| 24 | 20.9 | 15.1 |
| 25 | 11.0 | 13.8 |
| 26 | 26.5 | 13.2 |
| 27 | 23.0 | 11.0 |
| 28 | 21.2 | 10.3 |

C. Solid-State NMR

The ¹³C CPMAS of Compound I Phosphate Salt Form C (FIG. 107) was acquired at 275 K with 12.5 kHz spinning and using adamantane as a reference. The peaks are listed in Table 85 below.

TABLE 85
Peak list from 13C CPMAS of Compound I Phosphate Salt Form C

| Peak # | Chem Shift [ppm] | Intensity [rel] |
|---|---|---|
| 1 | 149.4 | 19.2 |
| 2 | 148.1 | 18.7 |
| 3 | 147.7 | 9.8 |
| 4 | 147.0 | 31.8 |
| 5 | 146.4 | 20.3 |
| 6 | 145.8 | 33.1 |
| 7 | 144.0 | 17.1 |
| 8 | 143.2 | 47.4 |
| 9 | 142.3 | 28.8 |
| 10 | 141.9 | 27.9 |
| 11 | 140.9 | 24.4 |
| 12 | 140.3 | 27.8 |
| 13 | 137.4 | 42.1 |
| 14 | 137.1 | 47.4 |
| 15 | 136.6 | 26.8 |
| 16 | 135.7 | 15.7 |
| 17 | 135.2 | 27.4 |
| 18 | 134.3 | 39.0 |
| 19 | 133.0 | 9.7 |
| 20 | 130.0 | 47.2 |
| 21 | 129.0 | 48.9 |
| 22 | 128.3 | 49.9 |
| 23 | 127.1 | 34.0 |
| 24 | 126.3 | 31.7 |
| 25 | 126.0 | 29.8 |
| 26 | 125.4 | 34.0 |
| 27 | 124.1 | 18.1 |
| 28 | 123.3 | 15.7 |
| 29 | 123.1 | 16.5 |
| 30 | 121.7 | 8.4 |
| 31 | 121.5 | 8.6 |
| 32 | 120.8 | 8.9 |
| 33 | 74.6 | 55.1 |
| 34 | 73.8 | 45.6 |
| 35 | 73.4 | 36.7 |
| 36 | 72.3 | 100.0 |
| 37 | 71.7 | 20.5 |
| 38 | 67.3 | 47.1 |
| 39 | 66.8 | 25.9 |
| 40 | 66.5 | 25.02 |
| 41 | 65.6 | 36.0 |
| 42 | 63.9 | 16.2 |
| 43 | 63.1 | 43.0 |
| 44 | 62.2 | 33.9 |
| 45 | 61.7 | 28.8 |
| 46 | 61.3 | 32.2 |
| 47 | 61.0 | 37.0 |
| 48 | 51.2 | 32.9 |
| 49 | 50.7 | 24.6 |
| 50 | 49.9 | 31.5 |
| 51 | 49.5 | 28.3 |
| 52 | 48.9 | 31.0 |
| 53 | 47.6 | 70.0 |
| 54 | 46.8 | 75.7 |
| 55 | 44.7 | 26.8 |
| 56 | 43.5 | 29.7 |
| 57 | 43.0 | 21.7 |
| 58 | 42.4 | 12.2 |
| 59 | 41.5 | 38.4 |
| 60 | 41.0 | 36.2 |
| 61 | 39.7 | 56.9 |
| 62 | 38.9 | 55.3 |
| 63 | 38.6 | 46.2 |
| 64 | 38.0 | 50.28 |
| 65 | 36.7 | 11.5 |
| 66 | 35.9 | 31.64 |
| 67 | 35.5 | 33.4 |
| 68 | 34.6 | 36.3 |
| 69 | 32.9 | 33.8 |
| 70 | 32.5 | 21.0 |
| 71 | 21.6 | 8.2 |
| 72 | 20.5 | 43.4 |
| 73 | 19.7 | 24.7 |
| 74 | 19.0 | 15.5 |
| 75 | 18.2 | 80.0 |
| 76 | 17.5 | 25.7 |
| 77 | 16.6 | 14.2 |

The ¹⁹F MAS of Compound I Phosphate Salt Form C (FIG. 108) was acquired at 275 K with 12.5 kHz spinning and using adamantane as a reference. The peaks are listed in Table 86 below. Additionally, the ³¹P CPMAS of Compound I Phosphate Salt Form C (FIG. 109) was acquired at 275 K with 12.5 kHz spinning and using adamantane as a reference. The peaks are listed in Table 87 below.

TABLE 86
Peak list from 19F MAS of Compound I Phosphate Salt Form C

| Peak # | Chem Shift [ppm] | Intensity [rel] |
|---|---|---|
| 1 | −56.6 | 5.3 |
| 2 | −57.6 | 12.5 |
| 3 | −58.3 | 9.3 |
| 4 | −59.0 | 2.6 |

TABLE 87
Peak list from 31P CPMAS of Compound I Phosphate Salt Form C

| Peak # | Chem Shift [ppm] | Intensity [rel] |
|---|---|---|
| 1 | 5.3 | 17.3 |
| 2 | 4.3 | 100.0 |
| 3 | 3.2 | 24.9 |
| 4 | 2.3 | 29.3 |
| 5 | 1.5 | 36.3 |
| 6 | 0.6 | 7.1 |

D. Thermogravimetric Analysis

Thermal gravimetric analysis of Compound I Phosphate Salt Form C was measured using the TA5500 Discovery TGA. A sample with weight of approximately 1-10 mg in an open platinum pan. The program was set to heat from ambient to 300° C. at a heating rate of 10° C. per min with nitrogen purge. The TGA thermogram shows 0.5% weight loss from ambient to 50° C. then minimal weight loss until 200° C. (FIG. 105).

E. Differential Scanning Calorimetry Analysis

DSC analysis of Compound I Phosphate Form C was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heat rate at of 2° C. per min to a temperature to 300° C. The thermogram shows two endotherm peaks at 113° C. and 184° C. (FIG. 106).

12. Compound I Phosphate Salt Crystalline Form Mixture

A. Synthetic Procedure

1 eq. of Compound I free form Monohydrate was charged to a reactor, followed by 5 vol. of 2-MeTHF. Agitation was commenced at 20° C. The temperature was raised to 30° C., and a clear solution was obtained in the reactor. In a separate container, 1.06 eq. of 85 wt % phosphoric acid was diluted with 2.7 vol. of 2-MeTHF. The phosphoric acid solution was then added to the reactor slowly over 2 hours. An additional 2 vol. of 2-MeTHF was added to make a thinner slurry. The final slurry was cooled back at 20° C. in 2 hours then held overnight and filtered under vacuum. The resulting wet cake was washed with 2 vol. of 2-MeTHF. The wet cake was dried under vacuum with a nitrogen bleed at 50° C. to yield Compound I Phosphate Salt Crystalline Form Mixture

B. X-Ray Powder Diffraction

The X-ray powder diffractogram of Compound I Phosphate Salt Crystalline Form Mixture was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step (FIG. 110).

TABLE 88
Peak list from XRPD diffractogram of Compound
I Phosphate Salt Crystalline Form Mixture

| No. | Pos. [±0.2, °2θ] | Rel. Int. [%] |
|---|---|---|
| 1 | 14.8 | 100.0 |
| 2 | 10.6 | 90.2 |
| 3 | 20.3 | 77.1 |
| 4 | 21.0 | 70.3 |
| 5 | 13.3 | 69.1 |
| 6 | 21.9 | 65.9 |
| 7 | 8.8 | 56.9 |
| 8 | 14.9 | 55.0 |
| 9 | 20.0 | 38.7 |
| 10 | 18.1 | 37.5 |
| 11 | 7.3 | 36.9 |
| 12 | 20.8 | 36.7 |
| 13 | 26.8 | 36.5 |
| 14 | 9.0 | 32.7 |
| 15 | 18.3 | 29.7 |
| 16 | 27.1 | 29.4 |
| 17 | 22.4 | 27.2 |
| 18 | 18.9 | 25.4 |
| 19 | 16.1 | 24.9 |
| 20 | 24.8 | 23.3 |
| 21 | 22.7 | 23.0 |
| 22 | 23.9 | 22.2 |
| 23 | 14.6 | 22.0 |
| 24 | 3.7 | 20.8 |
| 25 | 15.4 | 20.2 |
| 26 | 23.6 | 20.1 |
| 27 | 17.8 | 18.5 |
| 28 | 25.8 | 17.8 |
| 29 | 8.6 | 14.9 |
| 30 | 12.4 | 14.3 |
| 31 | 10.0 | 12.6 |
| 32 | 19.3 | 12.4 |
| 33 | 24.4 | 11.5 |
| 34 | 21.2 | 11.3 |
| 35 | 28.5 | 10.1 |

C. Solid-State NMR

The ¹³C CPMAS of Compound I Phosphate Salt Crystalline Form Mixture (FIG. 113) was acquired at 275 K with 12.5 kHz spinning and using adamantane as a reference. The peaks are listed in Table 89 below.

TABLE 89
Peak list from 13C CPMAS of Compound I
Phosphate Salt Crystalline Form Mixture

| Peak # | Chem Shift [ppm] | Intensity [rel] |
|---|---|---|
| 1 | 146.3 | 28.6 |
| 2 | 145.8 | 6.8 |
| 3 | 144.4 | 15.3 |
| 4 | 142.9 | 55.4 |
| 5 | 141.8 | 12.8 |
| 6 | 141.3 | 9.8 |
| 7 | 140.4 | 31.9 |
| 8 | 140.0 | 22.6 |
| 9 | 130.1 | 17.6 |
| 10 | 129.1 | 34.7 |
| 11 | 127.6 | 33.2 |
| 12 | 126.8 | 30.3 |
| 13 | 124.2 | 4.4 |
| 14 | 121.3 | 3.2 |
| 15 | 74.1 | 11.3 |
| 16 | 73.4 | 100.0 |
| 17 | 66.8 | 24.4 |
| 18 | 65.9 | 18.2 |
| 19 | 65.0 | 11.5 |
| 20 | 64.2 | 35.6 |
| 21 | 63.5 | 22.7 |
| 22 | 50.7 | 30.0 |
| 23 | 48.2 | 38.0 |
| 24 | 47.9 | 39.4 |
| 25 | 45.4 | 33.2 |
| 26 | 43.9 | 29.5 |
| 27 | 40.9 | 6.8 |
| 28 | 38.6 | 34.5 |
| 29 | 37.8 | 41.9 |
| 30 | 37.4 | 45.3 |
| 31 | 15.8 | 60.2 |
| 32 | 15.7 | 63.4 |

The ¹⁹F MAS of Compound I Phosphate Salt Crystalline Form Mixture (FIG. 114) was acquired at 275 K with 12.5 kHz spinning and using adamantane as a reference. The peaks are listed in Table 90 below. Additionally, the ³¹P CPMAS of Compound I Phosphate Salt Crystalline Form Mixture (FIG. 115) was acquired at 275 K with 12.5 kHz spinning and using adamantane as a reference. The peaks are listed in Table 91 below.

TABLE 90
Peak list from 19F MAS of Compound I Phosphate
Salt Crystalline Form Mixture

| Peak # | Chem Shift [ppm] | Intensity [rel] |
|---|---|---|
| 1 | −54.2 | 12.5 |
| 2 | −57.0 | 12.3 |

TABLE 91
Peak list from 31P CPMAS of Compound I
Phosphate Salt Crystalline Form Mixture

| Peak # | Chem Shift [ppm] | Intensity [rel] |
|---|---|---|
| 1 | 6.4 | 25.2 |
| 2 | 5.0 | 50.7 |
| 3 | 4.0 | 100.0 |
| 4 | 3.5 | 40.0 |

D. Thermogravimetric Analysis

Thermal gravimetric analysis of Compound I Phosphate Salt Crystalline Form Mixture was measured using the TA5500 Discovery TGA. A sample with weight of approximately 1-10 mg was scanned from ambient to 300° C. at a heating rate of 10° C./min with nitrogen purge. The TGA thermogram shows gradual weight loss of 0.9% from ambient temperature until 200° C. (FIG. 111).

E. Differential Scanning Calorimetry Analysis

The Differential Scanning calorimetric analysis of Compound I Phosphate Salt Crystalline Form Mixture was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The program was set to modulate 0.32° per 60 seconds, then heat rate at of 2° C. per min to a temperature to 300° C. The thermogram shows one endotherm peak at 237° C. (FIG. 112).

### Example 3: Synthesis of Compound II

### 1. Preparation of Compound II Synthetic Precursors

### Preparation of S1

### 2-(3-thienyl)ethanol (S1

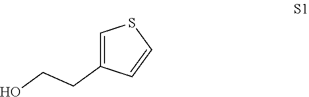

2-(3-thienyl)ethanol (S1) was obtained from commercial sources.

### Preparation of S2

### 2-(5-chloro-3-thienyl)ethanol (S2

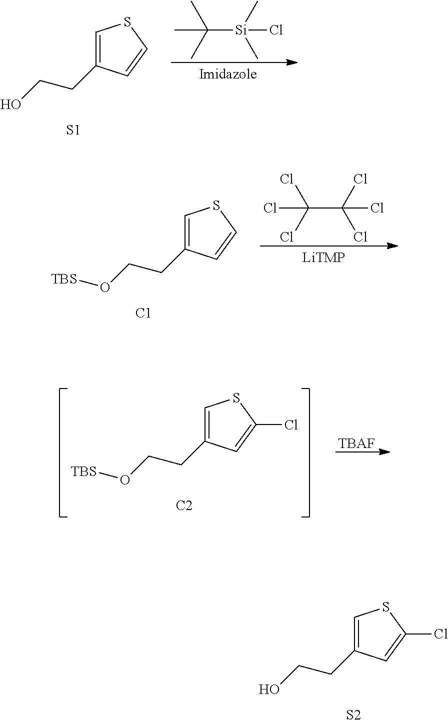

### Step 1. Synthesis of tert-butyl-dimethyl-[2-(3-thienyl)ethoxy]silane (C

To a solution of 2-(3-thienyl)ethanol S1 (18 g, 140.4 mmol) in DMF (100 mL) was added imidazole (12 g, 176.3 mmol) and tert-butyl-chloro-dimethyl-silane (24 g, 159.2 mmol) sequentially. Optionally, the alcohol may be protected as a triphenyl (trityl) ether or other suitable alcohol protecting group. An exotherm was observed. The reaction mixture was stirred at room temperature for 3 hours. The reaction mixture was diluted with MTBE (500 mL) and washed with water (200 mL), 0.5 N HCl (200 mL), water (200 mL), and brine (200 mL). The organic layer was dried, filtered, and concentrated in vacuo. The organic layer was dissolved in heptane and passed through a silica gel plug, which was washed with 1-5% MTBE/Heptane. Solvent was removed to afford tert-butyl-dimethyl-[2-(3-thienyl)ethoxy]silane C1 (34 g, 99%). ¹H NMR (400 MHz, Chloroform-d) δ 7.28-7.13 (m, 1H), 7.04-6.91 (m, 2H), 3.80 (t, J=6.9 Hz, 2H), 2.90-2.75 (m, 2H), 0.88 (s, 9H), −0.00 (s, 6H).

### Step 2. Synthesis of tert-butyl-[2-(5-chloro-3-thienyl)ethoxy]-dimethyl-silane (C2

To a solution of 2,2,6,6-tetramethylpiperidine (36 mL, 213.3 mmol) in tetrahydrofuran (200 mL) cooled to 0° C. was added a solution of hexyllithium (92 mL of 2.3 M, 211.6 mmol). The reaction was stirred for 30 minutes at −78° C. A solution of tert-butyl-dimethyl-[2-(3-thienyl)ethoxy]silane C1 (34 g, 138.8 mmol) in THF (150 mL) was added to the reaction over 20 minutes. The reaction was stirred at −30° C. for 45 minutes. The reaction was cooled to −78° C. and 1,1,1,2,2,2-hexachloroethane (54 g, 228.1 mmol) was added portion-wise. Other sources of electrophilic chloride may optionally be used for this step. The reaction was warmed to room temperature and stirred overnight. The reaction was quenched with saturated ammonium chloride (125 mL), diluted with water (100 mL), extracted with EtOAc (500 mL), and back extracted with EtOAc (100 mL). The combined organic layers were washed with 0.5 N HCl (200 mL), water (300 mL), and brine (200 mL). The organic layer was dried over sodium sulfate, filtered, and concentrated to afford the crude product tert-butyl-[2-(5-chloro-3-thienyl)ethoxy]-dimethyl-silane C2.

### Step 3. Synthesis of 2-(5-chloro-3-thienyl)ethanol (S2

To a solution of tert-butyl-[2-(5-chloro-3-thienyl)ethoxy]-dimethyl-silane C2 (12.5 g, 42.89 mmol) in 2-Me-THF (120 mL) was added TBAF (63 mL of 1 M in THF, 63.00 mmol). These conditions may be adjusted depending on the nature of the alcohol protecting group. The reaction was stirred at room temperature overnight. The reaction was partitioned between EtOAc (400 mL) and water (400 mL). The layers were separated, and the organic layer was extracted with EtOAc (200 mL), dried over sodium sulfate, filtered, and concentrated in vacuo. Purification by silica gel chromatography (Gradient: 0-50% EtOAc in heptane) yielded the product 2-(5-chloro-3-thienyl)ethanol S2 (4.5 g, 58%). ¹H NMR (300 MHz, Chloroform-d) δ 6.82 (d, J=0.9 Hz, 2H), 3.89-3.71 (m, 2H), 2.79 (t, J=6.4 Hz, 2H), 2.05 (s, 1H). LCMS m/z 162.91 [M+H]⁺.

### Preparation of L1

### (2'S,6'S, 7S)-2-chloro-2′-methyl-6′-(1-methyltriazol-4-yl)spiro[4,5-dihydrothieno[2,3-c]pyran-7,4′-piperidine] (L1

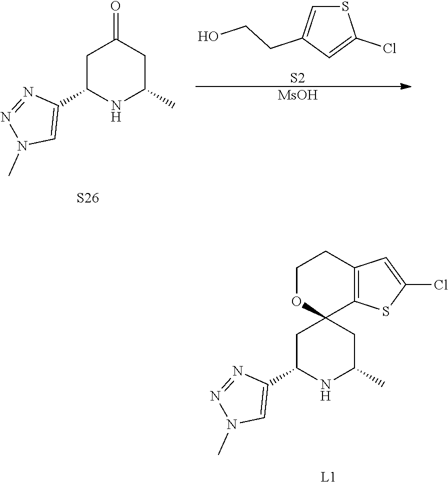

To a solution of (2S,6S)-2-methyl-6-(1-methyltriazol-4-yl)piperidin-4-one S26 (1380 mg, 7.11 mmol, prepared by Method A) in DCM (30 mL) was added 2-(5-chloro-3-thienyl)ethanol S2 (1100 μL, 8.894 mmol), followed by MsOH (3 mL, 46.23 mmol). The reaction was heated to reflux for 90 minutes, at which time it was cooled to room temperature and quenched with 2 N NaOH until the pH reached 14. The mixture was diluted with DCM (20 mL), and the organic layer was separated, washed with brine (30 mL), dried over MgSO₄, and concentrated in vacuo. Purification by silica gel chromatography (Gradient: 0-25% of 20% MeOH/DCM in DCM) yielded the product (2'S,6'S,7S)-2-chloro-2′-methyl-6′-(1-methyltriazol-4-yl)spiro[4,5-dihydrothieno[2,3-c]pyran-7,4′-piperidine] L1 (1162 mg, 48%) as a pale yellow oil in a >8:1 ratio. The minor isomer observed is inferred to be the enantiomer of L1, since S26 prepared by Method A contains minor quantities of the other cis enantiomer. Note that relative stereochemistry in L1 was assigned through NOE NMR studies. ¹H NMR (400 MHz, Chloroform-d) δ 7.42 (s, 1H), 6.58 (s, 1H), 4.41 (dd, J=11.8, 2.6 Hz, 1H), 4.06 (s, 3H), 4.02-3.86 (m, 2H), 3.30 (ddt, J=12.7, 6.3, 3.2 Hz, 1H), 2.70-2.49 (m, 2H), 2.35 (dt, J=13.6, 2.6 Hz, 1H), 2.06 (dt, J=13.7, 2.5 Hz, 1H), 1.79 (dd, J=13.6, 11.8 Hz, 1H), 1.42 (dd, J=13.7, 11.3 Hz, 1H), 1.31-1.19 (m, 1H), 1.12 (d, J=6.4 Hz, 3H). LCMS m/z 339.0 [M+H]⁺.

### Alternative Preparation of L1 (HCl salt

### (2'S,6'S, 7S)-2-chloro-2′-methyl-6′-(1-methyltriazol-4-yl)spiro[4,5-dihydrothieno[2,3-c]pyran-7,4′-piperidine] hydrochloride salt (L1

To (2S,6S)-2-methyl-6-(1-methyltriazol-4-yl)piperidin-4-one S26 (205 mg, 1.055 mmol) in DCM (5 mL) was added 2-(5-chloro-3-thienyl)ethanol S2 (150 μL, 1.213 mmol), followed by MsOH (300 μL, 4.623 mmol). The mixture was heated to reflux for 10 minutes, at which time it was cooled to room temperature and quenched with 2 N NaOH until the pH reached 14. The mixture was diluted with DCM (5 mL), and the organic layer was separated and concentrated in vacuo. Purification by silica gel chromatography (Gradient: 0-25% of 20% MeOH/DCM in DCM) yielded product which was immediately dissolved in minimal DCM and treated with HCl (100 μL of 4 M in dioxane, 0.4000 mmol). The mixture was concentrated in vacuo and the residue was azeotroped with DCM (5 mL) and dried to yield (2'S,6'S,7S)-2-chloro-2′-methyl-6′-(1-methyltriazol-4-yl)spiro[4,5-dihydrothieno[2,3-c]pyran-7,4′-piperidine](Hydrochloride salt) L1 (171.6 mg, 43%) as a pale yellow solid. ¹H NMR (300 MHz, DMSO-d₆) δ 9.46 (s, 1H), 9.24 (d, J=8.3 Hz, 1H), 8.29 (s, 1H), 6.95 (s, 1H), 4.67 (t, J=11.1 Hz, 1H), 4.09 (s, 3H), 3.95 (t, J=5.4 Hz, 2H), 3.72 (s, 1H), 2.61 (t, J=5.3 Hz, 2H), 2.46-2.32 (m, 2H), 2.25 (d, J=15.1 Hz, 1H), 2.01-1.86 (m, 1H), 1.29 (d, J=6.5 Hz, 3H). LCMS m/z 339.0 [M+H]⁺.

### Preparation of S33

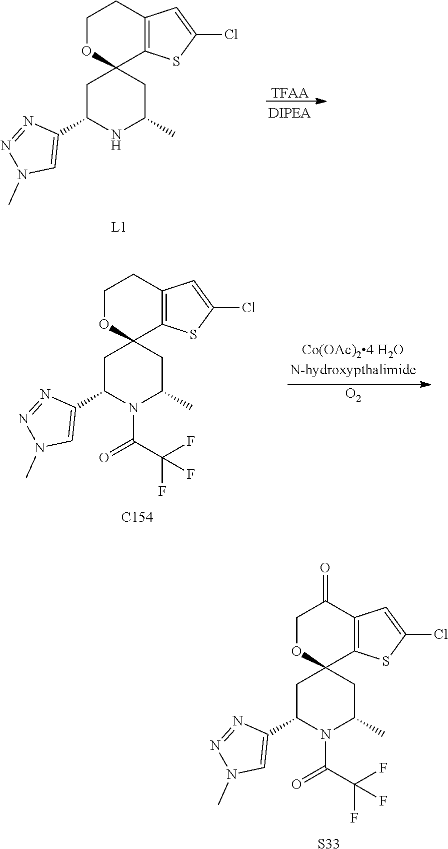

### Step 1. Synthesis of 1-[(2'S,6'S, 7S)-2-chloro-2′-methyl-6′-(1-methyltriazol-4- yl)spiro[4,5-dihydrothieno[2,3-c]pyran-7,4′-piperidine]-1′-yl]-²,2, 2-trifluoro-ethanone (C154

To a mixture of (2'S,6'S,7S)-2-chloro-2′-methyl-6′-(1-methyltriazol-4-yl)spiro[4,5-dihydrothieno[2,3-c]pyran-7,4′-piperidine] L1 (15.0 g, 43.82 mmol) and DIPEA (10 mL, 57.41 mmol) in DCM (150 mL), cooled to 3° C., was added TFAA (6.4 mL, 46.04 mmol). After 5 minutes, the mixture was quenched with 1 N HCl (100 mL), and the phases were separated. The organic layer was washed with brine (100 mL), dried with magnesium sulfate, filtered, and concentrated. The solid was suspended in TBME (100 mL) and heated to reflux. After 30 minutes, the mixture was cooled to 0° C., and after 10 minutes, the material was filtered and rinsed with additional cold TBME. The product was dried to yield 1-[(2'S,6'S,7S)-2-chloro-2′-methyl-6′-(1-methyltriazol-4- yl)spiro[4,5-dihydrothieno[2,3-c]pyran-7,4′-piperidine]-1′-yl]-2,2,2-trifluoro-ethanone C154 (15.532 g, 81%). LCMS m/z calc. 435.18 [M+H]⁺.

### Step 2. Synthesis of 2S,4S,6S)-2′-chloro-2-methyl-6-(1-methyltriazol-4-yl)-1-(2,2,2-trifluoroacetyl)spiro[piperidine-4,7′-thieno[2,3-c]pyran]-4′-one (S33

To a mixture of 1-[(2'S,6'S,7S)-2-chloro-2′-methyl-6′-(1-methyltriazol-4-yl)spiro[4,5-dihydrothieno[2,3-c]pyran-7,4′-piperidine]-1′-yl]-2,2,2-trifluoro-ethanone (C154) (4.5 g, 10.24 mmol) in acetonitrile (70 mL) was added N-hydroxyphthalimide (1.2 g, 7.36 mmol) and cobaltous diacetate tetrahydrate (550 mg, 0.216 mmol), and then the mixture was vacuum purged with an oxygen balloon three times. The mixture was heated to 45° C. and stirred for 18 hours before cooling to room temperature. The reaction was diluted with DCM, water, and saturated sodium bicarbonate, then extracted with DCM (3×150 mL) and collected through a phase separator. The organic layer was dried with Na₂SO₄, filtered, and concentrated. Purification by silica gel chromatography (Gradient: 0-50% EtOAc in heptane) afforded (2S,4S,6S)-2′-chloro-2-methyl-6-(1-methyltriazol-4-yl)-1-(2,2,2-trifluoroacetyl)spiro[piperidine-4,7′-thieno[2,3-c]pyran]-4′-one S33 (3.50 g, 68%). ¹H NMR (300 MHz, Chloroform-d) δ 7.61 (s, 1H), 7.19 (s, 1H), 5.61 (s, 1H), 4.44 (q, J=7.1 Hz, 1H), 4.31 (s, 2H), 4.12 (s, 3H), 3.34 (dd, J=15.1, 6.2 Hz, 1H), 2.78 (dd, J=15.1, 8.3 Hz, 1H), 2.70-2.43 (m, 1H), 2.16 (s, 1H), 1.27 (d, J=7.3 Hz, 3H). LCMS m/z 449.12 [M+H]⁺.

### 2. Synthesis of Compound II

### (2'S,4S,6'S, 7S)-2-chloro-2′-methyl-6′-(1-methyltriazol-4-yl)spiro[4,5-dihydrothieno[2,3-c]pyran-7,4′-piperidine]-4-ol (Compound II) Amorphous

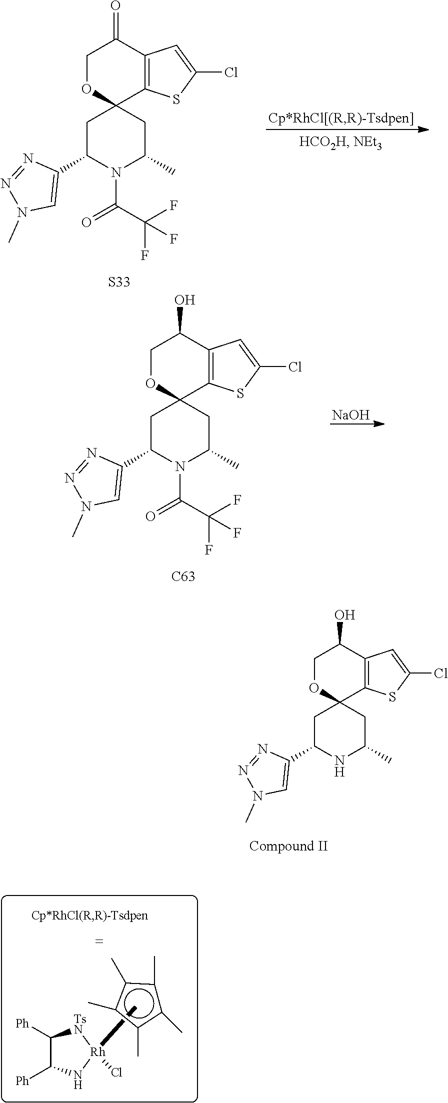

### Step 1. Synthesis of 1-[(2'S,4S,6'S, 7S)-2-chloro-4-hydroxy-2′-methyl-6′-(1-methyltriazol-4-yl)spiro[4,5-dihydrothieno[2,3-c]pyran-7,4′-piperidine]-1′-yl]-2,2,2-trifluoro-ethanone (C63

To (2S,4S,6S)-2′-chloro-2-methyl-6-(1-methyltriazol-4-yl)-1-(2,2,2-trifluoroacetyl)spiro[piperidine-4,7′-thieno[2,3-c]pyran]-4′-one S33 (3.5 g, 7.025 mmol) in DCM (60 mL) was added a solution of 1,2,3,4,5 pentamethylcyclopentane rhodium (2+) tetrachloride (24 mg, 0.03821 mmol) and N-[(1R,2R)-2-amino-1,2-diphenyl-ethyl]-4-methyl-benzenesulfonamide (27 mg, 0.074 mmol) in DCM (7 mL) followed by a solution of formic acid (1.4 mL, 37.11 mmol) and triethylamine (2.1 mL, 15.07 mmol). The flask was fitted with an empty balloon to capture the CO₂ off-gas byproduct. After two hours, the mixture was washed with saturated aqueous sodium bicarbonate (150 mL). The organic phase was separated, passed through a phase separator, and concentrated. Silica gel purification (Column: 120 g silica gel, Gradient: 0-45% EtOAc in Heptane) afforded 1-[(2'S,4S,6'S,7S)-2-chloro-4-hydroxy-2′-methyl-6′-(1-methyltriazol-4-yl)spiro[4,5-dihydrothieno[2,3-c]pyran-7,4′-piperidine]-1′-yl]-2,2,2-trifluoro-ethanone C63 as a pale off-white foam. ¹H NMR (300 MHz, Chloroform-d) δ 7.59 (s, 1H), 6.83 (s, 1H), 5.53 (s, 1H), 4.46 (dt, J=9.1, 3.1 Hz, 2H), 4.10 (s, 3H), 4.03-3.80 (m, 2H), 3.10 (dd, J=15.1, 7.3 Hz, 1H), 2.65 (ddd, J=15.1, 8.1, 2.2 Hz, 1H), 2.47 (s, 1H), 2.21-2.08 (m, 1H), 2.08 (d, J=9.2 Hz, 1H), 1.40-1.19 (m, 3H). LCMS m/z 451.05 [M+H]⁺.

Note that stereochemistry of alcohol C63 was assigned using NMR NOE studies and literature understanding of reductions using this catalyst and ligand system. (Reference: New Chiral Rhodium and Iridium Complexes with Chiral Diamine Ligands for Asymmetric Transfer Hydrogenation of Aromatic Ketones. Kunihiko Murata, Takao Ikariya, and Ryoji Noyori. The Journal of Organic Chemistry 1999 64 (7), 2186-2187).

### Step 2. Synthesis of (2'S,4S,6'S, 7S)-2-chloro-2′-methyl-6′-(1-methyltriazol-4-yl)spiro[4,5-dihydrothieno[2,3-c]pyran-7,4′-piperidine]-4-ol (Compound II

To a solution of 1-[(2'S,4S,6'S,7S)-2-chloro-4-hydroxy-2′-methyl-6′-(1-methyltriazol-4-yl)spiro[4,5-dihydrothieno[2,3-c]pyran-7,4′-piperidine]-1′-yl]-2,2,2-trifluoro-ethanone C63 (3.33 g, 100%) in MeOH (50 mL) was added NaOH (40 mL of 2 M, 80.00 mmol) and the mixture was stirred at 60° C. After 40 minutes, the mixture was diluted with saturated aqueous ammonium chloride until pH 10 (about 50 mL) and extracted with MTBE (5×100 mL) and ethyl acetate (1×75 mL). The combined organic layers were washed with saturated aqueous NaCl, dried over Na₂SO₄, and concentrated. The residue was brought up in EtOH and stripped down (3×) to afford a white solid. The solid was transferred to a vial and dried under vacuum at 55° C. overnight to give amorphous (2'S,4S,6'S,7S)-2-chloro-2′-methyl-6′-(1-methyltriazol-4-yl)spiro[4,5-dihydrothieno[2,3-c]pyran-7,4′-piperidine]-4-ol (Compound II) (2.1817 g, 87%). ¹H NMR (400 MHz, Methanol-d4) δ 7.82 (s, 1H), 6.88 (s, 1H), 4.46 (t, J=3.8 Hz, 1H), 4.34-4.28 (m, 1H), 4.08 (s, 3H), 4.04 (dd, J=12.2, 3.6 Hz, 1H), 3.81 (dd, J=12.2, 4.1 Hz, 1H), 3.36-3.25 (m, 1H), 2.39 (dt, J=13.8, 2.6 Hz, 1H), 2.17 (dt, J=13.7, 2.6 Hz, 1H), 1.71 (dd, J=13.9, 11.9 Hz, 1H), 1.45 (dd, J=13.7, 11.4 Hz, 1H), 1.16 (d, J=6.4 Hz, 3H). LCMS m/z 355.03 [M+H]⁺.

### Preparation of Compound II Free Form Hemihydrate Form A

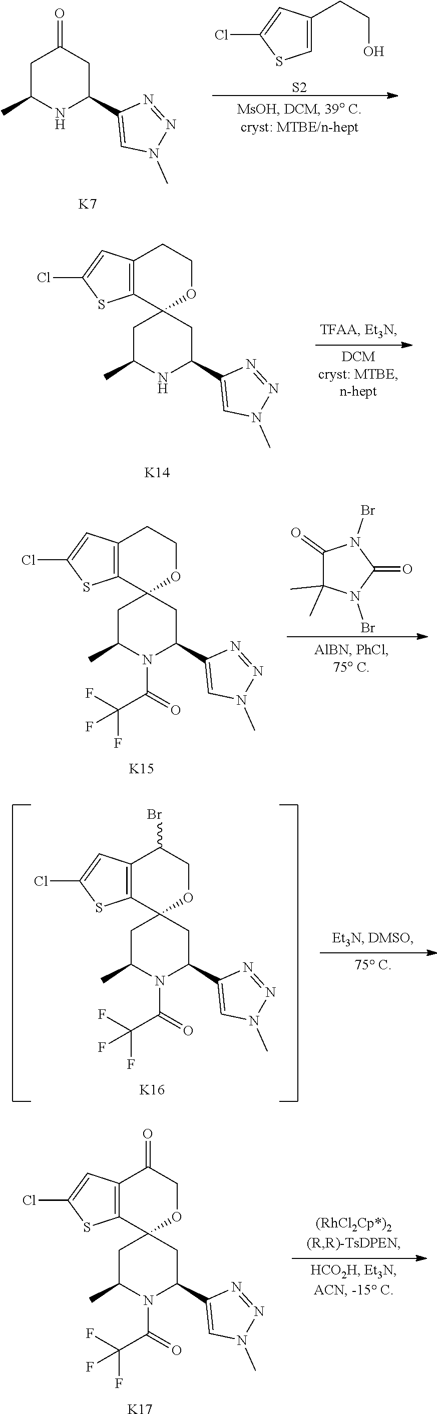

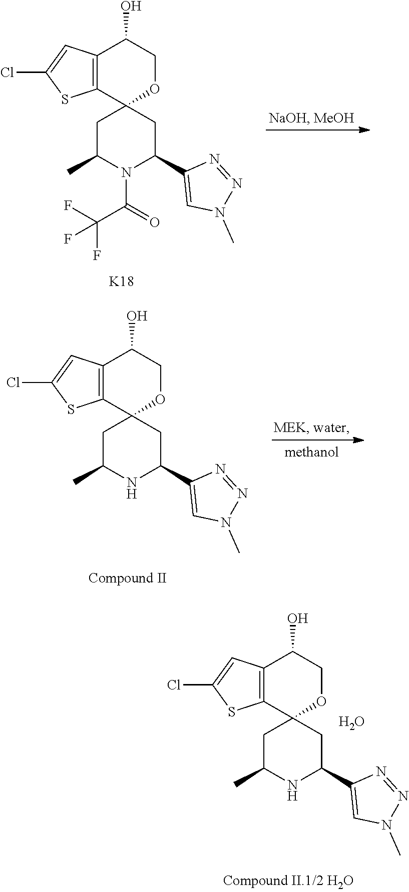

Step 1. A solution of K7 (4153 g, 1 equiv, 81.11% purity by qNMR, 21.53 mmol, 1 equiv) and K8 (3651 g, 22.45 mmol, 1.05 equiv) in dichloromethane (33.2 L, 8 vol) was treated with methanesulfonic acid (14384 g, 149.7 mol, 7 equiv) at 0° C. over 1 hour. The resulting mixture was heated at 40° C. After 14 hours, analysis showed >99% consumption of K7. The reaction mixture was cooled to 10° C. and adjusted to pH 10 with 4 N sodium hydroxide (40 L). The organic layer was separated, dried over sodium sulfate (1.5 kg), and evaporated under reduced pressure at 25° C. to give crude K14 as an off-white solid (8.1 kg). This solid was suspended in methyl tert-butyl ether (22 L), stirred at 10° C. for 2.5 hours, and then filtered. The filter cake was washed with methyl tert-butyl ether (4 L) and dried under vacuum while flushing with nitrogen at 20° C. for 18 hours to give purified 5950 g K14 (96.8% yield).

Step 2. A solution of K14 (5937 g, 17.52 mol, 1 equiv) and N,N-diisopropylethylamine (3967 mL, 22.78 mol, 1.3 equiv) in dichloromethane (59 L, 10 vol) was cooled to 0-5° C. and treated with trifluoroacetic acid anhydride (2680 mL, 19.27 mol, 1.1 equiv) over 40 minutes while keeping the reaction temperature below 14° C. The resulting reaction mixture was stirred at 0-10° C. After 2 hours, HPLC analysis indicated >99.5% conversion. The reaction mixture was cooled to 5° C. and diluted with saturated brine (27 L). The resulting mixture was adjusted to pH 10 with 6 N sodium hydroxide solution (5 L) while keeping the temperature below 12° C. The resulting mixture was stirred for 20 minutes, then the layers were separated. The organic layer was sequentially washed with 2 N HCl (3×22 L), water (3×22 L), and brine (22 L), then dried over sodium sulfate (1 kg) and evaporated under reduced pressure at 30° C. to give crude K15 (7519 g). The crude material was suspended in a mixture of methyl tert-butyl ether (16 L) and n-heptane (8 L) at 50° C. for 5 hours, then cooled to 20° C. over 5 hours. After 18 hours of stirring at 20° C., the suspension was filtered. The filter cake was washed with a mixture of methyl tert-butyl ether (8 L) and n-heptane (4 L), then dried under vacuum while flushing with nitrogen at 20° C. for 18 hours to give 6824 g of K15 (94.1% yield).

Steps 3 and 4. A suspension of K15 (5879 g, 13.52 mol, 1 equiv), azobisisobutyronitrile (178 g, 1.082 mol, 0.08 equiv), and 1,3-dibromo-5,5-dimethylhydantoin (2900 g, 10.14 mol, 0.75 equiv) in chlorobenzene (41.2 L, 7 vol) was sparged with nitrogen for 20 minutes in a 100 L jacketed glass reactor. The reaction mixture was then heated to 70° C. After 30 minutes, HPLC analysis indicated >99% conversion to K16. The reaction was cooled to 45° C., treated with anhydrous dimethylsulfoxide (41.2 L, 7 vol) and triethylamine (9.42 L, 67.55 mol, 5 equiv), and heated at 65° C. After 12 hours, HPLC analysis indicated complete consumption of K16. The reaction mixture was cooled to 0° C. and divided into two equal halves. Each half was treated with ice cold water (22 L), keeping the temperature below 15° C., then extracted with ethyl acetate (2×20 L). The aqueous layers were extracted with ethyl acetate (18 L). The combined organic layers were washed with water (2×24 L), brine (24 L), dried over anhydrous sodium sulfate (2 kg) then evaporated under reduced pressure at 50° C. to give a semi-solid residue which was co-evaporated with methanol (2×4 L) at 50° C. to give the crude product (6.65 kg) as a dark brown solid. The residue was triturated with methanol (32 L) at 65° C. for 5 hours, cooled to 15° C. over 5 hours, then filtered to give 3643 g of K17. This solid was triturated with a 1:2 mixture of acetone and methanol (18 L) at 65° C. for 5 hours, cooled to 20° C. over 5 hours, then filtered. The filter cake was rinsed with a 1:2 mixture of acetone and methanol (2×3 L), followed by methanol (3 L) at 20° C. The product was dried under nitrogen convection at 20° C. for 18 hours to give 2780 g of K₁₇ (45.8% yield).

Step 5. A solution of N-[(1R,2R)-2-amino-1,2-diphenyl-ethyl]-4-methyl-benzenesulfonamide (22.1 g, 0.06 mol, 0.01 equiv) and dichloro-(1,2,3,4,5-pentamethylcyclopenta-2,4-dien-1-yl)rhodium dimer (18.26 g, 0.03 mol, 0.005 equiv) in acetonitrile (12 L) was stirred for 30 minutes at 20° C., then cooled to −5° C. This solution was added to a suspension of K17 (2704 g, 6.024 mol, 1 equiv) in acetonitrile (16 L) and a mixture of formic acid (1.25 L, 33.13 mol, 5.5 equiv) and triethylamine (1.85 L, 13.25 mol, 2.2 equiv) (premixed and precooled to 0° C.) at 0° C. The resulting mixture was stirred at 0° C. and the progress of the reaction was monitored by HPLC. After 31 hours, HPLC analysis indicated >99.9% conversion to K18. The reaction mixture was diluted with a solution of sodium bicarbonate (2.1 kg) in water (30 L). The resulting mixture was stirred for 15 minutes at 10° C., then warmed to 15° C. and diluted with methyl tert-butyl ether (12 L). The resulting mixture was stirred for 15 minutes at 15° C. The layers were separated and the aqueous layer was extracted with methyl tert-butyl ether (12 L). The combined organic layers were sequentially washed with 1N HCl (2×11 L) and brine (2×11 L). During the second brine wash, the pH of the brine layer was adjusted to −8 using solid sodium bicarbonate (288 g). The organic layer was dried over anhydrous sodium sulfate (2 kg) and evaporated under reduced pressure to give 3.4 kg of crude K18. A solution of crude K18 in methyl tert-butyl ether (35 L) was treated with SiliaMetS DMT (1.7 kg) at 20° C. for 18 hours, then filtered. The filter cake was rinsed with methyl tert-butyl ether (5 L). The combined filtrates were treated with SiliaMetS DMT (1.7 kg, 0.5 vol) in 3 consecutive runs at 50° C. for 5 hours. The mixture was cooled to 20° C. in between treatments and filtered. The filtrate after final trituration was evaporated at 45° C. under reduced pressure to give 2.4 kg of K18 (68.9% yield).

Step 6. A solution of K18 (1785.8 g (corrected for NMR purity), 3.96 mol) in methanol (12.5 L, 7 vol) at 20° C. was treated with 6 N sodium hydroxide (5.0 L, 29.71 mol, 7.5 equiv, precooled to 5° C.) added in 4 equal portions over 20 minutes in a 100 L jacketed glass reactor. The resulting solution was stirred at 40° C. After 1.5 hours, LC-MS analysis indicated >99.9% conversion. The reaction mixture was cooled to 5-10° C. and adjusted to pH 10 to 11 with 6 N HCl (4 L). The reaction mixture was partially evaporated under reduced pressure at 37° C. to remove methanol. The mixture was diluted with isopropyl acetate (18 L) and water (2 L). The resulting suspension was heated to 46° C. to give clear phases. After stirring for 15 minutes at 46° C., the layers were separated. The aqueous layer was extracted with isopropyl acetate (10 L) at 40° C. The combined organic layers were washed with half saturated brine (10 L), followed by water (5 L) at 40° C. The organic layer was evaporated under reduced pressure at 40° C. to dryness to give 1298 g of crude Compound II (˜92% yield).

Step 7. Compound II (1207 g, 3.4 mol (corrected for ˜90% ¹HNMR purity), 1 equiv) was co-evaporated with methyl ethyl ketone (4 L) at 40° C. under reduced pressure. The residue was dissolved in methyl ethyl ketone (6 L) and filtered (˜8 μm porosity). The filtrate was charged to the reactor along with water (40 mL, 2.2 mol, 0.65 equiv). The resulting solution was heated to 60-62° C. n-Heptane (6 L, 5 vol) was charged to the hot solution over an hour, maintaining the temperature at 60-62° C. The resulting mixture was seeded (1 g, ˜0.1% wt) and heated at 62° C. for an hour. The resulting solution was cooled to 20° C. over 5 hours. After stirring at 20° C. for 18 hours, the suspension was filtered through Whatman #113 filter paper at 20° C. The filter cake was washed with a 4:1 mixture of n-heptane and methyl ethyl ketone (3 L) in 2 equal portions. The product was dried under nitrogen convection at 20° C. for 3 hours to give 1091.5 g of Compound II free form Hemihydrate Form A (Compound II.0.5 H₂O) as a white powder (86.1% yield).

### Example 4: Solid Forms of Compound II

### Solid State NMR Experimental—Applies to all Solid Forms of Compound II Disclosed Herein

A Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C and ³¹P cross-polarization (CP) MAS experiments. The fluorine relaxation time was measured using ¹⁹F MAS T₁ saturation recovery relaxation experiment in order to set up proper recycle delay of the ¹⁹F MAS experiment. The CP contact time of carbon as well as phosphorus CPMAS experiments was set to 2 ms. A CP proton pulse with linear ramp (from 50% to 100%) was employed. The carbon Hartmann-Hahn match was optimized on external reference sample (glycine), while phosphorus Hartmann-Hahn match was optimized on the actual samples. All carbon, phosphorus and fluorine spectra were recorded with proton decoupling using TPPM15 decoupling sequence with the field strength of approximately 100 kHz.

1. Compound II Phosphate Salt Hemihydrate Form A

### A. Synthetic Procedure

628 mg of Compound II free form Hemihydrate Form A was weighed in a 10 mL vial, followed by the addition of approximately 7.6 mL 2-MeTHF. About 3.7 mL of 0.5 M H₃PO₄, pre-formulated via mixing of about 0.42 mL 6 M H₃PO₄ (aq.) and about 4.6 mL MeOH, was added to the vial dropwise. The contents of the vial were stirred with a magnetic stirring bar at ambient temperature for two days, then the solids were collected via centrifugation and dried in a 40° C. vacuum oven overnight. 670 mg of total solids (Compound II Phosphate Salt Hemihydrate Form A) were recovered.

Alternatively, 1 eq. of Compound II free form Hemihydrate Form A was charged to a reactor, followed by 8 vol. of 2-MeTHF. The reaction mixture was agitated and heated to 40° C. The clear solution obtained at 40° C. was seeded with 1 wt % of Compound II Phosphate Salt Hemihydrate Form A. In a separate container, 1.02 eq. of 85 wt % phosphoric acid was diluted with 0.35 vol. of water, 3 vol. of 2-MeTHF, and 0.6 vol. of acetone. The phosphoric acid solution was then added to the reactor slowly over 2 hours. The resulting slurry was cooled to 20° C. over 5 hours. The final slurry was agitated at 20° C. for not less than 2 hours, then filtered under vacuum. The resulting wet cake was washed with 3 vol. of 2-MeTHF. The wet cake was dried under vacuum with a nitrogen bleed at 50° C. to yield ˜94% of Compound II Phosphate Salt Hemihydrate Form A.

### B. X-Ray Powder Diffraction

X-ray powder diffraction (XRPD) spectra were recorded at room temperature (25±2° C.) in transmission mode using a PANalytical Empyrean system equipped with a sealed tube source and a PIXcel 3D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 2θ with a step size of 0.0131303° and 49 s per step. The XRPD diffractogram for Compound II Phosphate Salt Hemihydrate Form A is provided in FIG. 24 and the data is summarized below in Table 39.

TABLE 39
Peak list from XRPD diffractogram of Compound
II Phosphate Hemihydrate Form A

| No. | Pos. [±0.2, °2θ] | Rel. Int. [%] |
|---|---|---|
| 1 | 9.1 | 100.0 |
| 2 | 16.7 | 77.4 |
| 3 | 18.7 | 68.1 |
| 4 | 20.0 | 43.3 |
| 5 | 15.7 | 41.9 |
| 6 | 14.9 | 39.0 |
| 7 | 18.4 | 36.1 |
| 8 | 10.1 | 32.8 |
| 9 | 20.2 | 32.4 |
| 10 | 15.2 | 27.0 |
| 11 | 23.9 | 25.7 |
| 12 | 20.7 | 25.6 |
| 13 | 23.6 | 24.6 |
| 14 | 16.3 | 23.9 |
| 15 | 17.1 | 23.4 |
| 16 | 21.0 | 21.4 |
| 17 | 26.2 | 20.5 |
| 18 | 22.0 | 20.4 |
| 19 | 21.2 | 19.8 |
| 20 | 19.8 | 19.0 |
| 21 | 27.4 | 18.1 |
| 22 | 17.8 | 18.0 |
| 23 | 10.2 | 17.1 |
| 24 | 21.6 | 16.6 |
| 25 | 24.1 | 15.8 |
| 26 | 13.2 | 15.3 |
| 27 | 25.5 | 14.9 |
| 28 | 25.7 | 14.8 |
| 29 | 18.9 | 12.5 |
| 30 | 20.4 | 12.0 |
| 31 | 22.7 | 11.8 |
| 32 | 22.3 | 11.7 |
| 33 | 17.9 | 11.1 |
| 34 | 8.8 | 11.0 |
| 35 | 19.6 | 10.6 |
| 36 | 27.0 | 10.5 |
| 37 | 10.5 | 10.3 |
| 38 | 27.2 | 10.1 |

### C. Solid-State NMR

The ¹³C CPMAS of Compound II Phosphate Salt Hemihydrate Form A (FIG. 25) was acquired at 275 K with 12.5 kHz spinning and using adamantane as a reference. The peaks are listed in Table 26 below. Additionally, the ¹³C CPMAS of Compound II Phosphate Salt Hemihydrate Form A following dehydration (FIG. 26) was acquired at 275 K with 12.5 kHz spinning and using adamantane as a reference. The peaks are listed in Table 40 below.

TABLE 40
Peak list from ¹³C CPMAS of Compound
II Phosphate Salt Hemihydrate Form A

| Peak # | Chem Shift [ppm] | Intensity [rel] |
|---|---|---|
| 1 | 144.7 | 17.8 |
| 2 | 143.5 | 16.3 |
| 3 | 142.9 | 20.7 |
| 4 | 141.3 | 53.4 |
| 5 | 140.6 | 4.3 |
| 6 | 140.2 | 19.6 |
| 7 | 139.7 | 20.0 |
| 8 | 139.1 | 21.1 |
| 9 | 136.8 | 30.3 |
| 10 | 135.9 | 27.0 |
| 11 | 129.5 | 20.2 |
| 12 | 127.6 | 22.6 |
| 13 | 127.1 | 29.4 |
| 14 | 126.6 | 30.0 |
| 15 | 125.6 | 24.1 |
| 16 | 125.1 | 21.1 |
| 17 | 123.7 | 20.8 |
| 18 | 73.0 | 36.2 |
| 19 | 72.5 | 100.0 |
| 20 | 66.1 | 29.9 |
| 21 | 65.4 | 32.8 |
| 22 | 63.4 | 32.1 |
| 23 | 62.8 | 17.5 |
| 24 | 61.4 | 28.2 |
| 25 | 50.5 | 62.2 |
| 26 | 48.4 | 30.7 |
| 27 | 47.7 | 41.4 |
| 28 | 46.9 | 34.6 |
| 29 | 43.9 | 21.2 |
| 30 | 42.6 | 21.0 |
| 31 | 40.8 | 20.6 |
| 32 | 40.5 | 21.9 |
| 33 | 39.9 | 31.2 |
| 34 | 39.4 | 20.8 |
| 35 | 39.0 | 26.3 |
| 36 | 38.6 | 28.0 |
| 37 | 37.4 | 23.9 |
| 38 | 36.7 | 24.6 |
| 39 | 36.1 | 23.7 |
| 40 | 34.6 | 22.34 |
| 41 | 18.4 | 27.3 |
| 42 | 16.6 | 34.58 |
| 43 | 15.8 | 31.5 |
| 44 | 15.3 | 35.4 |

TABLE 41
Peak list from ¹³C CPMAS of Dehydrated Compound
II Phosphate Hemihydrate Form A

| Peak # | Chem Shift [ppm] | Intensity [rel] |
|---|---|---|
| 1 | 144.7 | 23.3 |
| 2 | 144.1 | 18.6 |
| 3 | 143.0 | 33.0 |
| 4 | 141.3 | 31.2 |
| 5 | 140.7 | 17.7 |
| 6 | 140.2 | 27.1 |
| 7 | 139.5 | 14.1 |
| 8 | 138.6 | 16.1 |
| 9 | 138.3 | 18.2 |
| 10 | 136.8 | 29.8 |
| 11 | 129.0 | 22.5 |
| 12 | 127.5 | 53.1 |
| 13 | 125.6 | 42.3 |
| 14 | 124.7 | 27.6 |
| 15 | 123.9 | 22.3 |
| 16 | 73.3 | 100.0 |
| 17 | 73.0 | 55.6 |
| 18 | 72.2 | 58.5 |
| 19 | 66.5 | 60.3 |
| 20 | 65.0 | 46.4 |
| 21 | 64.1 | 57.4 |
| 22 | 62.4 | 10.5 |
| 23 | 61.2 | 46.6 |
| 24 | 50.2 | 34.2 |
| 25 | 48.5 | 60.9 |
| 26 | 47.6 | 39.0 |
| 27 | 46.5 | 39.4 |
| 28 | 46.1 | 29.9 |
| 29 | 45.3 | 26.1 |
| 30 | 44.0 | 20.1 |
| 31 | 42.9 | 46.8 |
| 32 | 40.6 | 32.6 |
| 33 | 39.3 | 53.4 |
| 34 | 38.5 | 54.1 |
| 35 | 37.0 | 33.9 |
| 36 | 36.6 | 29.5 |
| 37 | 34.5 | 21.6 |
| 38 | 16.5 | 89.0 |

The ³¹P CPMAS of Compound II Phosphate Salt Hemihydrate Form A (FIG. 27A) was acquired at 275 K with 12.5 kHz spinning and using adamantane as a reference. The peaks are listed in Table 42A below. Additionally, the ³¹P CPMAS of Compound II Phosphate Salt Hemihydrate Form A following dehydration (FIG. 27B) was acquired at 275 K with 12.5 kHz spinning and using adamantane as a reference. The peaks are listed in Table 42B below.

TABLE 42A
Peak list from ³¹P CPMAS of Compound
II Phosphate Hemihydrate Form A

| Peak # | Chem Shift [ppm] | Intensity [rel] |
|---|---|---|
| 1 | 3.1 | 100.0 |
| 2 | −1.1 | 70.6 |
| 3 | −1.8 | 28.0 |

TABLE 42B
Peak list from ³¹P CPMAS of Dehydrated Compound
II Phosphate Hemihydrate Form A

| Peak # | Chem Shift [ppm] | Intensity [rel] |
|---|---|---|
| 1 | 5.6 | 81.9 |
| 2 | 4.4 | 100.0 |
| 3 | 3.2 | 85.0 |
| 4 | 3.0 | 96.7 |

### D. Thermogravimetric Analysis

Thermal gravimetric analysis of Compound II Phosphate Salt Hemihydrate Form A was conducted using a TA Discovery 550 TGA from TA Instrument. A sample with a weight of 1-10 mg was scanned from 25° C. to 350° C. at a heating rate of 10° C./min with nitrogen purge. Data were collected by Thermal Advantage Q Series' software and analyzed by Trios and/or Universal Analysis software (TA Instruments, New Castle, Del.). The thermogram showed 2.4% weight loss from ambient temperature up to 150° C. (FIG. 28).

### E. Differential Scanning Calorimetry Analysis

DSC of Compound II Phosphate Salt Hemihydrate Form A was conducted using a TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 250° C. When the run was completed, the data were analyzed by Trios and/or Universal Analysis software (TA Instruments, New Castle, Del.). The thermogram showed two endothermic peaks around 123° C. and 224° C. (FIG. 29).

### F. Single Crystal Elucidation

Single crystals having the Compound II Phosphate Salt Hemihydrate Form A structure were grown from a mixture of 2-MeTHF, water, and acetone. X-ray diffraction data were acquired at 100 K on a Bruker diffractometer equipped with Cu K_{α} radiation (λ=1.54178 Å) and a CMOS detector. The structure was solved and refined using SHELX programs (Sheldrick, G. M., Acta Cryst., (2008) A64, 112-122). The results are summarized in Table 43 below.

TABLE 43
Single crystal elucidation of Compound
II Phosphate Salt Hemihydrate Form A

| Crystal System | Orthorhombic |
|---|---|
| Space Group | P2₁2₁2₁ |
| a (Å) | 9.2337(3) |
| b (Å) | 23.4759(9) |
| c (Å) | 38.3254(12) |
| α (°) | 90 |
| β (°) | 90 |
| γ (°) | 90 |
| V (Å³) | 8307.8(5) |
| Z/Z′ | 4/4 |
| Temperature | 100 K |

2. Compound II Free Form Hemihydrate Form A

### A. Synthetic Procedure

100 mg of Amorphous free form Compound II was added to a glass vial. To this was added 0.4 mL MEK, and all solids dissolved. 3 μL water was then added to aid the hemihydrate formation. To this mixture was added 0.25 mL of n-Heptane directly. After stirring for 18 hours at ambient temperature, the solids were filtered, rinsing with 1:4 MEK/n-Heptane (v/v), followed by 100% n-Heptane. The solids were collected, dried in a vacuum oven (60° C.) overnight, and characterized.

### B. X-Ray Powder Diffraction

X-ray powder diffraction (XRPD) spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96-well sample holder with mylar film and loaded into the instrument. The sample was scanned over the range of about 3° to about 40° 2θ with a step size of 0.0131303° and 49 s per step. The XRPD diffractogram for Compound II free form Hemihydrate Form A is provided in FIG. 30A and the data is summarized below in Table 44.

TABLE 44
Peak list from XRPD diffractogram of Compound II
free form Hemihydrate Form A (Room Temperature)

| No. | Pos. [±0.2, °2θ] | Rel. Int. [%] |
|---|---|---|
| 1 | 17.1 | 100.0 |
| 2 | 20.4 | 87.3 |
| 3 | 19.1 | 74.1 |
| 4 | 6.5 | 66.2 |
| 5 | 5.7 | 46.8 |
| 6 | 14.4 | 32.6 |
| 7 | 12.1 | 25.6 |
| 8 | 11.4 | 22.3 |
| 9 | 25.5 | 22.0 |
| 10 | 12.3 | 20.5 |
| 11 | 18.9 | 19.9 |
| 12 | 9.4 | 19.9 |
| 13 | 22.4 | 19.7 |
| 14 | 21.8 | 18.8 |
| 15 | 15.8 | 17.7 |
| 16 | 22.7 | 14.5 |
| 17 | 22.4 | 14.2 |
| 18 | 20.8 | 12.9 |
| 19 | 25.0 | 12.4 |
| 20 | 26.1 | 12.1 |
| 21 | 29.0 | 12.0 |
| 22 | 26.1 | 11.8 |
| 23 | 27.0 | 11.8 |
| 24 | 19.3 | 11.5 |
| 25 | 25.1 | 11.2 |
| 26 | 25.3 | 11.1 |
| 27 | 6.2 | 10.9 |
| 28 | 27.9 | 10.9 |
| 29 | 28.4 | 10.7 |
| 30 | 15.7 | 10.0 |

Additionally, in one test of in situ variable temperature XRPD (VT-XRPD), Compound II free form Hemihydrate Form A was observed to show peak shifts at elevated temperature. Variable temperature X-ray powder diffraction (VT-XRPD) spectra were recorded in 30-90° C. in reflection mode using a PANalytical Empyrean system equipped with a sealed tube source and a PIXcel 1D Medipix-2 detector (Malvern PANalytical Inc, Westborough, Mass.). The step-wise temperature change in increments of 10° C. from 30° C. to 90° C. with a hold at each temperature for 1 hour, followed by XRD collection. The sample chamber was purged with house nitrogen. The X-Ray generator operated at a voltage of 45 kV and a current of 40 mA with copper radiation (1.54060 Å). The sample was scanned over the range of about 3° to about 40° 20 with a step size of 0.0131303° and 49.725 s per step.

Three distinct XRPD patterns were found, respectively, at: (1) ambient temperature to 30° C.; (2) 40-50° C.; and (3) 60-90° C. The sample returned to its initial form after re-equilibration at ambient temperature and humidity. The XRPD spectrum from ambient temperature to 30° C. was the same (within ±0.2° 20) as the XRPD spectrum collected at room temperature (25±2° C.). Table 45 lists the peaks observed between 40-50° C., and Table 46 lists the peaks observed between 60-90° C.

TABLE 45
Peak list from XRPD diffractogram of Compound
II free form Hemihydrate Form A (40-50° C.)

| No. | Pos. [±0.2, °2θ] | Rel. Int. [%] |
|---|---|---|
| 1 | 20.1 | 100.0 |
| 2 | 19.0 | 85.2 |
| 3 | 11.3 | 58.1 |
| 4 | 5.6 | 56.8 |
| 5 | 22.3 | 56.2 |
| 6 | 25.1 | 45.7 |
| 7 | 24.8 | 43.1 |
| 8 | 27.8 | 42.5 |
| 9 | 22.1 | 40.3 |
| 10 | 17.2 | 32.5 |
| 11 | 9.5 | 30.8 |
| 12 | 11.9 | 29.1 |
| 13 | 18.7 | 28.0 |
| 14 | 15.6 | 23.2 |
| 15 | 20.9 | 22.6 |
| 16 | 6.6 | 22.5 |
| 17 | 21.9 | 21.6 |
| 18 | 23.9 | 21.4 |
| 19 | 22.6 | 21.2 |
| 20 | 29.9 | 20.6 |
| 21 | 19.6 | 20.1 |
| 22 | 30.0 | 19.8 |
| 23 | 26.6 | 19.7 |
| 24 | 25.9 | 19.5 |
| 25 | 28.9 | 18.4 |
| 26 | 26.2 | 17.1 |
| 27 | 26.9 | 16.3 |
| 28 | 27.0 | 16.2 |
| 29 | 28.7 | 15.6 |
| 30 | 19.3 | 15.3 |
| 31 | 28.3 | 14.5 |
| 32 | 14.4 | 12.4 |
| 33 | 17.8 | 12.3 |
| 34 | 25.5 | 11.6 |
| 35 | 23.4 | 11.0 |
| 36 | 23.1 | 11.0 |
| 37 | 29.4 | 10.9 |
| 38 | 24.2 | 10.6 |

TABLE 46
Peak list from XRPD diffractogram of Compound
II free form Hemihydrate Form A (60-90° C.)

| No. | Pos. [±0.2, °2θ] | Rel. Int. [%] |
|---|---|---|
| 1 | 19.8 | 100.0 |
| 2 | 19.2 | 72.1 |
| 3 | 5.5 | 62.0 |
| 4 | 21.8 | 60.5 |
| 5 | 11.0 | 50.3 |
| 6 | 27.2 | 48.3 |
| 7 | 24.7 | 46.0 |
| 8 | 19.0 | 44.5 |
| 9 | 22.0 | 40.6 |
| 10 | 24.3 | 40.4 |
| 11 | 17.3 | 35.5 |
| 12 | 11.7 | 28.8 |
| 13 | 9.6 | 28.7 |
| 14 | 29.3 | 24.2 |
| 15 | 29.3 | 23.4 |
| 16 | 21.0 | 21.8 |
| 17 | 26.8 | 20.7 |
| 18 | 23.5 | 20.5 |
| 19 | 15.5 | 20.0 |
| 20 | 6.7 | 19.9 |
| 21 | 27.4 | 18.0 |
| 22 | 25.1 | 17.7 |
| 23 | 25.8 | 16.4 |
| 24 | 23.0 | 15.8 |
| 25 | 29.9 | 15.1 |
| 26 | 14.4 | 14.8 |
| 27 | 25.9 | 14.4 |
| 28 | 28.3 | 14.3 |
| 29 | 17.8 | 13.2 |
| 30 | 15.6 | 13.0 |
| 31 | 25.6 | 12.3 |
| 32 | 20.3 | 12.3 |
| 33 | 22.6 | 12.2 |

### C. Solid-State NMR

The ¹³C CPMAS of Compound II free form Hemihydrate Form A (FIG. 31) was acquired at 275 K with 12.5 kHz spinning and using adamantane as a reference. The peaks are listed in Table 47 below.

TABLE 47
Peak list from ¹³C CPMAS of Compound
II free form Hemihydrate Form A

| Peak # | Chem Shift [ppm] | Intensity [rel] |
|---|---|---|
| 1 | 150.9 | 32.6 |
| 2 | 147.6 | 38.4 |
| 3 | 142.7 | 44.5 |
| 4 | 140.9 | 47.7 |
| 5 | 139.8 | 65.2 |
| 6 | 133.2 | 56.1 |
| 7 | 131.9 | 25.5 |
| 8 | 124.7 | 42.9 |
| 9 | 124.2 | 40.7 |
| 10 | 123.4 | 27.4 |
| 11 | 74.6 | 100.0 |
| 12 | 67.9 | 58.3 |
| 13 | 65.0 | 48.6 |
| 14 | 64.0 | 39.3 |
| 15 | 61.9 | 44.1 |
| 16 | 49.7 | 48.6 |
| 17 | 49.4 | 39.6 |
| 18 | 48.2 | 38.3 |
| 19 | 47.4 | 34.0 |
| 20 | 46.4 | 42.4 |
| 21 | 43.9 | 33.0 |
| 22 | 43.2 | 28.9 |
| 23 | 40.3 | 35.4 |
| 24 | 38.4 | 41.0 |
| 25 | 35.8 | 36.3 |
| 26 | 22.6 | 70.0 |
| 27 | 21.9 | 61.5 |

### D. Thermogravimetric Analysis

Thermal gravimetric analysis of Compound II free form Hemihydrate Form A was conducted using a TA Discovery 550 TGA from TA Instrument. A sample with a weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, Del.). The thermogram showed 2.4% weight loss from ambient temperature up to 150° C. (FIG. 33).

### E. Differential Scanning Calorimetry Analysis

DSC of Compound II free form Hemihydrate Form A was conducted using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, Del.). The thermogram showed endothermic peaks around 77° C., 107° C., and 125° C. (FIG. 34).

### F. Single Crystal Elucidation

Single crystals having the Compound II free form Hemihydrate Form A structure were grown from a mixture of chlorobenzene and hexanes at 4° C. X-ray diffraction data were acquired at 100 K on a Bruker diffractometer equipped with Cu K_{α} radiation (λ=1.54178 Å) and a CMOS detector. The structure was solved and refined using SHELX programs (Sheldrick, G. M., Acta Cryst., (2008) A64, 112-122). The results are summarized in Table 48 below.

TABLE 48
Single crystal elucidation of Compound
II free form Hemihydrate Form A

| Crystal System | Monoclinic |
|---|---|
| Space Group | P2₁ |
| a (Å) | 13.7571(9) |
| b (Å) | 8.1029(5) |
| c (Å) | 15.5760(11) |
| α (°) | 90 |
| β (°) | 100.220(4) |
| γ (°) | 90 |
| V (Å³) | 1708.7(2) |
| Z/Z′ | 2/2 |
| Temperature | 100 K |

3. Compound II Free Form Form C

### A. Synthetic Procedure

100 mg of Compound II free form Hemihydrate Form A was weighed in a vial followed by adding 0.5 ml MEK. Let the sample stir at 20° C. overnight and isolated the solids for form analysis.

Alternatively, Compound II free form Form C was made by the following procedure: 1.99 g Compound II free form Hemihydrate was measured to a 50 mL reactor equipped with overhead stirrer and temperature probe. 3.98 mL ethanol and 3.98 mL water were added to the reactor. Temperature was set to 55° C. After the system turned into clear solution, 0.019 g Compound II free form Form C was added to the reactor. 7.96 mL water was added over 30 minutes. The system was cooled to 20° C. over 1.5 hours and held at this temperature until the solid was isolated for analysis.

### B. X-Ray Powder Diffraction

X-ray powder diffraction spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound II free form Form C is provided in FIG. 35 and the data is summarized in Table 49.

TABLE 49
Peak list from XRPD diffractogram of Compound
II free form Form C (Room Temperature)

| No. | Pos. [±0.2, °2θ] | Rel. Int. [%] |
|---|---|---|
| 1 | 13.0 | 100.0 |
| 2 | 21.6 | 98.4 |
| 3 | 18.5 | 79.0 |
| 4 | 17.9 | 65.1 |
| 5 | 19.8 | 32.1 |
| 6 | 15.7 | 32.0 |
| 7 | 23.6 | 30.5 |
| 8 | 11.1 | 26.7 |
| 9 | 22.0 | 24.3 |
| 10 | 26.7 | 24.2 |
| 11 | 30.6 | 20.3 |
| 12 | 15.5 | 14.1 |
| 13 | 17.7 | 13.4 |
| 14 | 26.3 | 13.0 |
| 15 | 24.0 | 12.9 |
| 16 | 17.1 | 12.4 |
| 17 | 16.5 | 12.2 |
| 18 | 23.3 | 12.1 |
| 19 | 26.8 | 10.9 |

### C. Solid-State NMR

The ¹³C CPMAS of Compound II free form Form C (FIG. 38) was acquired at 275 K with 12.5 kHz spinning and using adamantane as a reference. The peaks are listed in Table 50 below.

TABLE 50
Peak List from ¹³C CPMAS of Compound II free form Form C

| Peak # | Chem Shift [ppm] | Intensity [rel] |
|---|---|---|
| 1 | 149.3 | 4.2 |
| 2 | 144.3 | 4.3 |
| 3 | 135.0 | 5.3 |
| 4 | 130.4 | 0.4 |
| 5 | 127.2 | 3.4 |
| 6 | 124.5 | 4.5 |
| 7 | 74.0 | 10.0 |
| 8 | 66.9 | 6.6 |
| 9 | 62.0 | 7.4 |
| 10 | 49.4 | 5.5 |
| 11 | 47.8 | 9.6 |
| 12 | 37.7 | 4.7 |
| 13 | 36.8 | 5.8 |
| 14 | 149.3 | 4.2 |

### D. Thermogravimetric Analysis

Thermal gravimetric analysis of Compound II free form Form C was measured using TGA Q5000 from TA Instrument. A sample with weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, Del.). The thermogram showed negligible weight loss from ambient temperature up to 200° C. (FIG. 36).

### E. Differential Scanning Calorimetry Analysis

DSC of Compound II free form Form C was measured using the TA Instruments Discovery DSC 2500. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, Del.). The thermogram showed an endothermic peak at 218° C. (FIG. 37).

### F. Single Crystal Elucidation

Single crystals having the Compound II free form Form C structure were grown by from MEK. X-ray diffraction data were acquired at 298 K on a Bruker diffractometer equipped with Cu Kα radiation (λ=1.54178 Å) and a CPAD detector. The structure was solved and refined using SHELX programs (Sheldrick, G. M., Acta Cryst., (2008) A64, 112-122) and results are summarized in Table 51 below.

TABLE 51
Single crystal elucidation of Compound II free form Form C

| Crystal System | Orthorhombic |
|---|---|
| Space Group | P2₁2₁2₁ |
| a (Å) | 10.3436(2) |
| b (Å) | 12.5318(2) |
| c (Å) | 12.8136(3) |
| α (°) | 90 |
| β (°) | 90 |
| γ (°) | 90 |
| V (Å³) | 1660.95(6) |
| Z/Z′ | 4/1 |
| Temperature | 298 K |

Single crystals having the Compound II free form Form C were grown by from MEK. X-ray diffraction data were acquired at 100 K on a Bruker diffractometer equipped with Cu Kα radiation (λ=1.54178 Å) and a CPAD detector. The structure was solved and refined using SHELX programs (Sheldrick, G. M., Acta Cryst., (2008) A64, 112-122) and results are summarized in Table 52 below.

TABLE 52
Single crystal elucidation of Compound II free form Form C

| Crystal System | Orthorhombic |
|---|---|
| Space Group | P2₁2₁2₁ |
| a (Å) | 10.2855(5) |
| b (Å) | 12.4759(6) |
| c (Å) | 12.6494(6) |
| α (°) | 90 |
| β (°) | 90 |
| γ (°) | 90 |
| V (Å³) | 1623.18(14) |
| Z/Z′ | 4/1 |
| Temperature | 100 K |

4. Compound II Free Form Form A

### A. Synthetic Procedure

Compound II free form Form A was obtained via desolvating MeOH solvate in 40° C. vacuum oven overnight or longer and isolating solids for form analysis.

### B. X-Ray Powder Diffraction

X-ray powder diffraction (XRPD) spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound II free form Form A is provided in FIG. 60 and the data is summarized in Table 53.

TABLE 53
Peak list from XRPD diffractogram
of Compound II free form Form A

| No. | Pos. [±0.2, °2θ] | Rel. Int. [%] |
|---|---|---|
| 1 | 9.1 | 100.0 |
| 2 | 16.6 | 91.2 |
| 3 | 11.7 | 76.6 |
| 4 | 13.9 | 54.7 |
| 5 | 22.1 | 53.3 |
| 6 | 20.5 | 51.0 |
| 7 | 14.1 | 50.5 |
| 8 | 18.3 | 49.2 |
| 9 | 24.4 | 34.7 |
| 10 | 17.3 | 32.4 |
| 11 | 23.2 | 29.2 |
| 12 | 10.6 | 25.6 |
| 13 | 22.7 | 25.5 |
| 14 | 23.5 | 24.5 |
| 15 | 23.8 | 21.7 |
| 16 | 8.3 | 19.2 |
| 17 | 27.2 | 18.4 |
| 18 | 23.6 | 18.2 |
| 19 | 18.0 | 13.3 |
| 20 | 15.5 | 13.1 |
| 21 | 26.0 | 10.5 |

### C. Solid-State NMR

The ¹³C CPMAS of Compound II free form Form A (FIG. 61) was acquired at 275 K with 12.5 kHz spinning and using adamantane as a reference. The peaks are listed in Table 54 below.

TABLE 54
Peak List from ¹³C CPMAS of Compound II free form Form A

| Peak # | Chem Shift [ppm] | Intensity [rel] |
|---|---|---|
| 1 | 147.4 | 54.3 |
| 2 | 143.6 | 55.2 |
| 3 | 134.1 | 63.6 |
| 4 | 128.8 | 49.0 |
| 5 | 123.4 | 46.0 |
| 6 | 74.0 | 100.0 |
| 7 | 68.3 | 75.1 |
| 8 | 62.0 | 83.0 |
| 9 | 48.9 | 65.0 |
| 10 | 48.1 | 60.6 |
| 11 | 46.9 | 61.0 |
| 12 | 39.6 | 51.6 |
| 13 | 39.1 | 65.1 |
| 14 | 21.6 | 73.9 |

### D. Thermogravimetric Analysis

Thermal gravimetric analysis of Compound II free form Form A was measured using Discovery TGA 5500 of TA Instrument. A sample with weight of approximately 1-10 mg was scanned from 25° C. to 300° C. at a heating rate of 10° C./min with nitrogen purge. Data were collected by TRIOS software and analyzed by TRIOS and/or Universal Analysis software (TA Instruments, New Castle, Del.). The thermogram showed negligible weight loss from ambient temperature up to 200° C. (FIG. 62).

### E. Differential Scanning Calorimetry Analysis

DSC of Compound II free form Form A was measured using the TA Instruments Discovery DSC 2500. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 2° C./min (modulated temperature amplitude at 0.3200° C., period 60 seconds) to 300° C. When the run was completed, the data were analyzed by Trios and/or Universal Analysis software (TA Instruments, New Castle, Del.). The thermogram showed an endothermic peak at 130° C. (FIG. 63).

### F. Single Crystal Elucidation

A single crystal having the Compound II free form Form A structure was obtained via slurry of Compound II free form Hemihydrate Form A in heptane at 80° C. X-ray diffraction data were acquired at 209 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122) and results are summarized in Table 55 below.

TABLE 55
Single crystal elucidation of Compound II free form Form A

| Crystal System | Monoclinic |
|---|---|
| Space Group | I2 |
| a (Å) | 10.0689(6) |
| b (Å) | 8.0474(5) |
| c (Å) | 21.7735(13) |
| α (°) | 90 |
| β (°) | 101.019(4) |
| γ (°) | 90 |
| V (Å³) | 1731.75(18) |
| Z/Z′ | 4/1 |
| Temperature (K) | 209(2) |

5. Compound II Free Form Form B

### A. Synthesis

Compound II free form Hemihydrate Form A was loaded into ssNMR rotor and dried at 80° C. oven overnight. The rotor was sealed with a cap soon before it left the drying oven for analysis.

B. Solid State NMR The ¹³C CPMAS of Compound II free form Form B was acquired at 275 K with 12.5 kHz spinning and using adamantane as a reference (FIG. 64). The peaks are listed in Table 56 below.

TABLE 56
Peak list from ¹³C CPMAS of Compound II free form Form B

| Peak # | Chem Shift [ppm] | Intensity [rel] |
|---|---|---|
| 1 | 151.9 | 20.5 |
| 2 | 147.2 | 17.3 |
| 3 | 142.2 | 23.6 |
| 4 | 141.5 | 25.6 |
| 5 | 139.4 | 34.1 |
| 6 | 132.9 | 29.6 |
| 7 | 130.7 | 14.4 |
| 8 | 125.5 | 21.1 |
| 9 | 124.4 | 24.0 |
| 10 | 121.1 | 17.5 |
| 11 | 74.6 | 100.0 |
| 12 | 67.6 | 39.4 |
| 13 | 64.1 | 58.3 |
| 14 | 61.8 | 30.9 |
| 15 | 49.9 | 24.8 |
| 16 | 49.2 | 25.8 |
| 17 | 47.7 | 42.9 |
| 18 | 47.3 | 39.9 |
| 19 | 46.6 | 30.3 |
| 20 | 45.2 | 26.8 |
| 21 | 44.3 | 33.9 |
| 22 | 39.8 | 26.2 |
| 23 | 38.5 | 26.3 |
| 24 | 35.3 | 22.2 |
| 25 | 22.6 | 41.1 |
| 26 | 22.4 | 43.6 |

### C. Single Crystal Elucidation

A single crystal having the Compound II free form Form B structure was obtained by holding a single crystal of Compound II free form Hemihydrate Form A under a nitrogen stream at 70° C. for 2 hours. X-ray diffraction data were acquired at 100 K on a Broker diffractometer equipped with Cu Kα radiation (λ=1.54178 Å) and a CMOS detector. The structure was solved and refined using SHELX programs (Sheldrick, G. M., Acta Cryst., (2008) A64, 112-122) and results are summarized in Table 57 below.

TABLE 57
Single crystal elucidation of Compound II free form Form B

| Crystal System | Monoclinic |
|---|---|
| Space Group | P2₁ |
| a (Å) | 13.3937(4) |
| b (Å) | 8.1370(2) |
| c (Å) | 15.9933(5) |
| α (°) | 90 |
| β (°) | 101.1224(19) |
| γ (°) | 90 |
| V (Å³) | 1710.28(9) |
| Z/Z′ | 2/2 |
| Temperature (K) | 100(2) |

6. Compound II Free Form Quarter Hydrate

### A. Synthesis

Compound II free form Hemihydrate Form A was dehydrated in isothermal 80° C. TGA for 1 hour, followed by unloading the solid to pack in the rotor as quickly as possible. The rotor was sealed with the cap soon after the solid was loaded.

### B. Solid State NMR

One partially dehydrated Compound II free form Hemihydrate Form A was captured on ssNMR as shown in the difference spectrum below. This partially dehydrated Hemihydrate A had z′ equal to 3 or 4, showing a similar trend of z′ increased relative to Compound II free form Hemihydrate Form A as the unit cell expansion observed in quarter hydrate on SCXRD. Therefore, the partially dehydrated free form Hemihydrate Form A on ssNMR has been identified as Compound II free form Quarter Hydrate.

Two spectra and chemical shift tables are shown below. The first is the spectrum of the ˜19% mixture of Compound II free form Quarter Hydrate with Compound II free form Hemihydrate Form A (as acquired at 275 K with 12.5 kHz spinning and using adamantane as a reference). The peaks are shown in FIG. 65 and listed in Table 58 below. The second is the spectrum of Quarter Hydrate only (after subtracting spectrum of the Hemihydrate Form A). The peaks are shown in FIG. 66 and listed in Table 59 below.

TABLE 58
Peak list from ¹³C CPMAS of ~19% physical
mixture of Compound II free form Quarter Hydrate
with Compound II free form Hemihydrate Form A

| Peak # | Chem Shift [ppm] | Intensity [rel] |
|---|---|---|
| 1 | 151.8 | 14.8 |
| 2 | 151.5 | 15.3 |
| 3 | 147.5 | 20.1 |
| 4 | 142.4 | 23.5 |
| 5 | 141.1 | 24.5 |
| 6 | 139.7 | 31.3 |
| 7 | 139.5 | 26.4 |
| 8 | 133.2 | 23.0 |
| 9 | 133.0 | 25.7 |
| 10 | 131.7 | 11.1 |
| 11 | 130.9 | 11.3 |
| 12 | 125.4 | 17.8 |
| 13 | 124.7 | 21.6 |
| 14 | 124.4 | 25.1 |
| 15 | 124.0 | 19.6 |
| 16 | 123.3 | 15.0 |
| 17 | 121.1 | 12.5 |
| 18 | 74.7 | 52.1 |
| 19 | 74.4 | 100.0 |
| 20 | 67.6 | 35.0 |
| 21 | 64.5 | 51.4 |
| 22 | 63.6 | 23.3 |
| 23 | 61.8 | 39.7 |
| 24 | 50.0 | 30.1 |
| 25 | 49.7 | 33.6 |
| 26 | 48.8 | 23.0 |
| 27 | 48.2 | 27.7 |
| 28 | 47.5 | 41.3 |
| 29 | 47.2 | 44.3 |
| 30 | 46.4 | 30.2 |
| 31 | 46.2 | 28.0 |
| 32 | 44.1 | 50.4 |
| 33 | 40.2 | 25.6 |
| 34 | 38.6 | 21.9 |
| 35 | 38.2 | 19.4 |
| 36 | 36.0 | 17.3 |
| 37 | 35.3 | 16.0 |
| 38 | 23.0 | 27.9 |
| 39 | 22.5 | 16.5 |
| 40 | 22.1 | 64.0 |

TABLE 59
Peak list from ¹³C CPMAS of Compound II free
form Quarter Hydrate after Subtraction of
Compound II free form Hemihydrate Form A

| Peak # | Chem Shift [ppm] | Intensity [rel] |
|---|---|---|
| 1 | 151.8 | 17.7 |
| 2 | 151.5 | 18.0 |
| 3 | 147.4 | 18.1 |
| 4 | 142.4 | 26.8 |
| 5 | 141.1 | 28.1 |
| 6 | 139.5 | 29.5 |
| 7 | 133.3 | 20.3 |
| 8 | 133.0 | 24.9 |
| 9 | 131.6 | 9.6 |
| 10 | 130.9 | 12.7 |
| 11 | 125.4 | 19.8 |
| 12 | 124.7 | 17.4 |
| 13 | 124.4 | 24.3 |
| 14 | 123.9 | 17.0 |
| 15 | 121.1 | 14.7 |
| 16 | 74.7 | 48.5 |
| 17 | 74.4 | 100.0 |
| 18 | 67.6 | 39.0 |
| 19 | 64.5 | 59.4 |
| 20 | 63.6 | 26.1 |
| 21 | 61.8 | 37.9 |
| 22 | 50.0 | 31.0 |
| 23 | 49.8 | 30.6 |
| 24 | 48.8 | 25.0 |
| 25 | 48.2 | 25.0 |
| 26 | 47.5 | 43.6 |
| 27 | 47.2 | 47.1 |
| 28 | 46.5 | 27.7 |
| 29 | 46.2 | 28.4 |
| 30 | 44.1 | 56.5 |
| 31 | 40.4 | 21.6 |
| 32 | 40.2 | 23.6 |
| 33 | 38.7 | 23.7 |
| 34 | 38.2 | 18.9 |
| 35 | 36.0 | 18.3 |
| 36 | 35.3 | 18.0 |
| 37 | 23.0 | 32.6 |
| 38 | 22.1 | 73.9 |

### C. Single Crystal Elucidation

A single crystal having the Compound II free form Quarter Hydrate structure was obtained by holding a single crystal of Compound II free form Hemihydrate Form A under a nitrogen stream at 40° C. for 40 minutes. X-ray diffraction data were acquired at 100 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122) and results are summarized in Table 60 below.

TABLE 60
Single crystal elucidation of Compound
II free form Quarter Hydrate

| Crystal System | Monoclinic |
|---|---|
| Space Group | P2₁ |
| a (Å) | 18.8543(6) |
| b (Å) | 8.1012(2) |
| c (Å) | 22.6144(7) |
| α (°) | 90 |
| β (°) | 99.143(3) |
| γ (°) | 90 |
| V (Å³) | 3410.30(17) |
| Z/Z′ | 2/4 |
| Temperature (K) | 100(2) |

7. Compound II Free Form Hydrate Mixture

### A. Synthetic Procedure

Compound II free form Form A was placed in a humidified chamber set at 95% RH for 3 days. The solid was collected and analyzed.

### B. X-Ray Powder Diffraction

X-ray powder diffraction (XRPD) diffractograms were recorded at room temperature in reflection mode using a PANalytical Empyrean system equipped with a sealed tube source and a PIXcel 1D Medipix-2 detector (Malvern PANalytical Inc, Westborough, Mass.). The X-ray generator operated at a voltage of 45 kV and a current of 40 mA with copper radiation (1.54060 Å). The powder sample was placed in a sample holder inside of a humidified chamber, which enabled precise controls of the chamber temperature and humidity. The whole unit was loaded into the instrument. The sample was scanned over the range of about 3° to about 40° 20 with a step size of 0.0131303° and 49.725 s per step.

The XRPD diffractogram for Compound II free form Hydrate Mixture is provided in FIG. 67 and the data is summarized in Table 61.

TABLE 61
Peak list from XRPD diffractogram of
Compund II free form Hydrate Mixture

| No. | Pos. [±0.2, °2θ] | Rel. Int. [%] |
|---|---|---|
| 1 | 22.2 | 100.0 |
| 2 | 21.6 | 66.4 |
| 3 | 8.6 | 51.8 |
| 4 | 17.0 | 39.7 |
| 5 | 24.1 | 33.6 |
| 6 | 14.6 | 33.2 |
| 7 | 24.5 | 29.1 |
| 8 | 22.6 | 28.6 |
| 9 | 16.7 | 23.2 |
| 10 | 13.7 | 21.8 |
| 11 | 3.6 | 20.3 |
| 12 | 15.9 | 18.0 |
| 13 | 28.0 | 16.3 |
| 14 | 30.4 | 15.7 |
| 15 | 22.8 | 15.0 |
| 16 | 23.1 | 14.8 |
| 17 | 20.0 | 14.4 |
| 18 | 19.9 | 13.7 |
| 19 | 27.8 | 13.2 |
| 20 | 24.7 | 12.9 |
| 21 | 30.5 | 12.7 |
| 22 | 30.0 | 12.5 |
| 23 | 17.3 | 12.3 |
| 24 | 12.2 | 10.6 |

### C. Solid State NMR

Compound II free form Monohydrate was made by humidifying Compound II free form Form A solids in 69% RH chamber equilibrating in saturated potassium iodide for 1-2 months under static conditions. The ¹³C CPMAS of Compound II free form Monohydrate (FIG. 68) was acquired at 275 K with 12.5 kHz spinning and using adamantane as a reference. The peaks for Compound II free form Monohydrate are listed in Table 62 below.

Compound II free form Dihydrate) was made by humidifying Compound II free form Form A solids in 94% RH chamber equilibrating with saturated potassium nitrate solution for 12 days under static conditions. This procedure provided a mixture of Compound II free form Dihydrate with about 29% free form Hemihydrate Form A and about 18% free form Form A. The ¹³C CPMAS of the mixture of Compound II free form Dihydrate with Compound II free form Hemihydrate Form A and Compound II free form Form A (FIG. 69) was acquired at 275 K with 12.5 kHz spinning and using adamantane as a reference. The peaks for the mixture are listed in Table 63 below. The peak list for pure Compound II free form Dihydrate (i.e., with the spectra for 29% Hemihydrate Form A and about 18% free form Form A subtracted out) is shown in FIG. 70 and Table 64 below.

TABLE 62
Peak list from ¹³C CPMAS of Compound
II free form Monohydrate

| Peak # | Chem Shift [ppm] | Intensity [rel] |
|---|---|---|
| 1 | 147.3 | 43.3 |
| 2 | 144.4 | 27.6 |
| 3 | 143.8 | 31.8 |
| 4 | 134.1 | 36.8 |
| 5 | 133.4 | 33.0 |
| 6 | 129.0 | 24.1 |
| 7 | 126.4 | 23.7 |
| 8 | 125.8 | 32.2 |
| 9 | 123.3 | 23.4 |
| 10 | 74.5 | 100.0 |
| 11 | 68.6 | 41.6 |
| 12 | 67.4 | 39.8 |
| 13 | 62.4 | 68.6 |
| 14 | 49.0 | 90.3 |
| 15 | 47.5 | 33.8 |
| 16 | 46.6 | 9.8 |
| 17 | 45.6 | 32.0 |
| 18 | 39.1 | 69.0 |
| 19 | 37.7 | 35.3 |
| 20 | 21.7 | 51.1 |
| 21 | 21.1 | 42.9 |

TABLE 63
Peak list from ¹³C CPMAS of Compound
II free form Dihydrate/Hemihydrate
Form A/free form Form A Mixture

| Peak # | Chem Shift [ppm] | Intensity [rel] |
|---|---|---|
| 1 | 150.7 | 10.6 |
| 2 | 147.3 | 46.6 |
| 3 | 144.5 | 35.2 |
| 4 | 143.8 | 10.8 |
| 5 | 142.6 | 12.7 |
| 6 | 140.7 | 12.6 |
| 7 | 139.6 | 16.7 |
| 8 | 133.6 | 41.7 |
| 9 | 131.8 | 13.6 |
| 10 | 128.9 | 9.7 |
| 11 | 126.6 | 31.2 |
| 12 | 125.5 | 37.9 |
| 13 | 124.5 | 19.5 |
| 14 | 124.1 | 17.2 |
| 15 | 123.3 | 18.7 |
| 16 | 74.8 | 68.5 |
| 17 | 74.5 | 69.2 |
| 18 | 68.6 | 17.1 |
| 19 | 67.8 | 69.8 |
| 20 | 64.8 | 18.4 |
| 21 | 63.8 | 15.6 |
| 22 | 62.7 | 57.6 |
| 23 | 61.8 | 18.0 |
| 24 | 49.4 | 100.0 |
| 25 | 48.2 | 20.1 |
| 26 | 47.4 | 23.9 |
| 27 | 46.3 | 51.9 |
| 28 | 45.5 | 14.1 |
| 29 | 43.8 | 14.0 |
| 30 | 43.0 | 12.2 |
| 31 | 40.3 | 14.8 |
| 32 | 39.0 | 25.7 |
| 33 | 38.2 | 57.1 |
| 34 | 37.8 | 53.2 |
| 35 | 35.7 | 14.6 |
| 36 | 22.5 | 22.0 |
| 37 | 21.5 | 56.3 |

TABLE 64
Peak list from ¹³C CPMAS of Compound II free form Dihydrate
After Subtraction of Compound II free form Hemihydrate
Form A and Compound II free form Form A

| Peak # | Chem Shift [ppm] | Intensity [rel] |
|---|---|---|
| 1 | 147.2 | 48.1 |
| 2 | 144.5 | 43.3 |
| 3 | 133.6 | 49.6 |
| 4 | 126.6 | 36.1 |
| 5 | 125.5 | 44.3 |
| 6 | 74.8 | 79.9 |
| 7 | 74.5 | 43.8 |
| 8 | 67.8 | 63.1 |
| 9 | 62.7 | 68.5 |
| 10 | 49.4 | 100.0 |
| 11 | 46.2 | 45.9 |
| 12 | 45.5 | 15.4 |
| 13 | 38.9 | 23.1 |
| 14 | 38.2 | 59.1 |
| 15 | 37.8 | 64.3 |
| 16 | 21.4 | 63.9 |

8. Compound II Free Form EtOH Solvate Form B

### A. Synthetic Procedure

Compound II free form EtOH Solvate Form B was prepared by slow evaporation of Compound II in EtOH at 4° C.

### B. X-Ray Powder Diffraction

XRPD was performed with a Panalytical X'Pert³ Powder XRPD on a Si zero-background holder. The 20 position was calibrated against a Panalytical Si reference standard disc. The parameters used are listed in the following table.

TABLE
Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2θ) | 3-40 |
| Scan step time [s] | 18.87 |
| Step size (°2θ) | 0.0131 |
| Test Time | 4 min 15 s |

The XRPD diffractogram for Compound II free form EtOH Solvate Form B is provided in FIG. 71 and the data is summarized in Table 65 below.

TABLE 65
Peak list from XRPD diffractogram of Compound II EtOH Solvate

| No. | Pos. [±0.2, °2θ] | Rel. Int. [%] |
|---|---|---|
| 1* | 11.6 | 100.0 |
| 2* | 23.8 | 38.5 |
| 3 | 23.7 | 34.2 |
| 4* | 16.6 | 28.6 |
| 5 | 17.1 | 27.0 |
| 6 | 7.6 | 18.7 |
| 7 | 23.3 | 14.9 |

### C. Thermogravimetric Analysis

Thermal gravimetric analysis of Compound II free form EtOH Solvate Form B was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, Del.). The thermogram showed about 9% weight loss from ambient temperature up to 200° C. (FIG. 72).

### D. Differential Scanning Calorimetry Analysis

DSC of Compound II free form EtOH Solvate Form B was measured using the TA Instruments Discovery DSC 2500. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, Del.). The thermogram showed endothermic peaks around 67° C. and 105° C. (FIG. 73).

9. Compound II Free Form IPA Solvate

### A. Synthetic Procedure

Compound II free form IPA Solvate was made via slurry of Compound II free form Hemihydrate Form A in 50/50 IPA/heptane (vol/vol).

### B. X-Ray Powder Diffraction

X-ray powder diffraction (XRPD) spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound II free form IPA Solvate is provided in FIG. 74 and the data is summarized in Table 66.

TABLE 66
Peak list from XRPD diffractogram of
Compound II free form IPA Solvate

| No. | Pos. [±0.2, °2θ] | Rel. Int. [%] |
|---|---|---|
| 1 | 23.3 | 100.0 |
| 2 | 11.7 | 78.4 |
| 3 | 21.6 | 57.7 |
| 4 | 8.4 | 51.0 |
| 5 | 22.1 | 50.2 |
| 6 | 17.0 | 45.3 |
| 7 | 19.9 | 44.5 |
| 8 | 21.9 | 41.1 |
| 9 | 17.6 | 35.8 |
| 10 | 7.4 | 28.6 |
| 11 | 23.1 | 25.7 |
| 12 | 17.5 | 25.1 |
| 13 | 25.4 | 24.8 |
| 14 | 16.7 | 24.2 |
| 15 | 15.5 | 18.6 |
| 16 | 12.6 | 15.6 |
| 17 | 20.4 | 11.7 |
| 18 | 23.5 | 11.1 |
| 19 | 22.7 | 11.1 |
| 20 | 26.4 | 10.2 |

### C. Solid-State NMR

The ¹³C CPMAS of Compound II free form IPA Solvate (FIG. 75) was acquired at 275 K with 12.5 kHz spinning and using adamantane as a reference. The peaks are listed in Table 67 below.

TABLE 67
Peak List from ¹³C CPMAS of Compound II free form IPA Solvate

| Peak # | Chem Shift [ppm] | Intensity [rel] |
|---|---|---|
| 1 | 150.9 | 35.2 |
| 2 | 147.5 | 78.8 |
| 3 | 143.0 | 51.7 |
| 4 | 140.6 | 34.1 |
| 5 | 139.6 | 44.7 |
| 6 | 135.4 | 38.2 |
| 7 | 132.9 | 42.7 |
| 8 | 131.0 | 34.6 |
| 9 | 128.3 | 33.1 |
| 10 | 125.2 | 38.9 |
| 11 | 124.8 | 46.3 |
| 12 | 123.8 | 45.9 |
| 13 | 74.9 | 68.4 |
| 14 | 74.5 | 100.0 |
| 15 | 68.3 | 43.5 |
| 16 | 67.8 | 26.9 |
| 17 | 64.6 | 45.3 |
| 18 | 63.8 | 46.8 |
| 19 | 61.7 | 73.8 |
| 20 | 49.5 | 99.9 |
| 21 | 48.9 | 52.3 |
| 22 | 48.3 | 46.3 |
| 23 | 47.4 | 39.0 |
| 24 | 46.3 | 43.3 |
| 25 | 46.0 | 48.1 |
| 26 | 43.7 | 35.7 |
| 27 | 43.0 | 31.6 |
| 28 | 40.3 | 40.6 |
| 29 | 39.9 | 41.4 |
| 30 | 38.2 | 44.2 |
| 31 | 37.6 | 41.9 |
| 32 | 35.7 | 38.4 |
| 33 | 33.3 | 30.6 |
| 34 | 30.5 | 14.7 |
| 35 | 25.3 | 15.1 |
| 36 | 24.2 | 28.2 |
| 37 | 22.4 | 60.0 |
| 38 | 22.0 | 66.6 |
| 39 | 21.7 | 56.8 |
| 40 | 15.1 | 40.2 |

10. Compound II Free Form MEK Solvate

### A. Synthetic Procedure

Compound II free form MEK Solvate was identified as a was found as the minor phase of Compound II free form Form C via the following procedure:

50.07 g Compound II free form Hemihydrate Form A was charged to 500 mL jacketed reactor equipped with a retreat curve mechanical stirrer, a huber ministat, a findenser, an N2 bubbler and a RX-10;

adding methyl ethyl ketone (250.35 mL) to the reactor;

setting reaction temperature to 45° C. and agitating at 300 rpm;

adding 0.488 g Compound II free form Hemihydrate Form A as seeds and holding temperature 45° C. for 30 minutes;

setting reaction temperature to 20° C. and cooling over 1 hour.

### B. Solid-State NMR

The ¹³C CPMAS of Compound II free form MEK Solvate (FIG. 76) was acquired at 275 K with 15 kHz spinning and using adamantane as a reference. The peaks are listed in Table 68 below.

TABLE 68
Peak List from ¹³C CPMAS of Compound
II free form MEK Solvate

| Peak # | Chem Shift [ppm] | Intensity [rel] |
|---|---|---|
| 1 | 63.3 | 0.2 |
| 2 | 39.3 | 0.2 |
| 3 | 35.7 | 0.2 |
| 4 | 35.0 | 0.2 |
| 5 | 30.0 | 0.1 |
| 6 | 23.2 | 0.1 |
| 7 | 8.2 | 0.2 |

11. Compound II Free Form MeOH Solvate

### A. Synthetic Procedure

Compound II free form MeOH Solvate was made by mixing Amorphous free form Compound II with MeOH at 200-300 mg/ml followed by rotatory evaporation.

### B. X-Ray Powder Diffraction

X-ray powder diffraction spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound II free form MeOH Solvate is provided in FIG. 77 and the data is summarized in Table 69.

TABLE 69
Peak list from XRPD diffractogram of
Compound II free form MeOH Solvate

| No. | Pos. [±0.2, °2θ] | Rel. Int. [%] |
|---|---|---|
| 1 | 24.3 | 100.0 |
| 2 | 26.3 | 48.0 |
| 3 | 13.4 | 31.8 |
| 4 | 24.4 | 30.0 |
| 5 | 16.6 | 29.8 |
| 6 | 12.0 | 28.0 |
| 7 | 24.1 | 26.5 |
| 8 | 24.2 | 26.3 |
| 9 | 21.2 | 24.2 |
| 10 | 15.8 | 21.3 |
| 11 | 8.0 | 20.6 |
| 12 | 21.1 | 20.4 |
| 13 | 16.0 | 18.4 |
| 14 | 22.9 | 16.8 |
| 15 | 23.9 | 11.6 |
| 16 | 24.6 | 10.9 |
| 17 | 19.7 | 10.5 |

### C. Solid-State NMR

The ¹³C CPMAS of Compound II free form MeOH Solvate (FIG. 78) was acquired at 275 K with 12.5 kHz spinning and using adamantane as a reference. The peaks are listed in Table 70 below.

TABLE 70
Peak List from ¹³C CPMAS of Compound
II free form MeOH Solvate

| Peak # | Chem Shift [ppm] | Intensity [rel] |
|---|---|---|
| 1 | 146.9 | 44.7 |
| 2 | 144.6 | 49.0 |
| 3 | 133.6 | 62.1 |
| 4 | 127.2 | 46.1 |
| 5 | 126.6 | 46.7 |
| 6 | 74.8 | 100.0 |
| 7 | 67.7 | 78.7 |
| 8 | 62.6 | 78.8 |
| 9 | 49.8 | 93.3 |
| 10 | 46.4 | 49.4 |
| 11 | 38.1 | 58.5 |
| 12 | 37.0 | 55.0 |
| 13 | 21.2 | 83.1 |

### D. Thermogravimetric Analysis

Thermal gravimetric analysis of Compound II free form MeOH Solvate was measured using TGA Q5000 from TA Instrument. A sample with weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, Del.). The thermogram showed 0.87% weight loss from ambient temperature up to 150° C. (FIG. 79).

### E. Differential Scanning Calorimetry Analysis

DSC of Compound II free form MeOH Solvate was measured using the TA Instruments Discovery DSC 2500. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, Del.). The thermogram showed endothermic peaks 79° C., 112° C., and 266° C. (FIG. 80).

### F. Single Crystal Elucidation

Single crystals having the Compound II free form MeOH Solvate structure were grown by slow evaporation from methanol. X-ray diffraction data were acquired at 100 K on a Bruker diffractometer equipped with Cu Kα radiation (λ=1.54178 Å) and a CMOS detector. The structure was solved and refined using SHELX programs (Sheldrick, G. M., Acta Cryst., (2008) A64, 112-122) and results are summarized Table 71 below.

TABLE 71
Single crystal elucidation of Compound II free form MeOH Solvate

| Crystal System | Monoclinic |
|---|---|
| Space Group | C2 |
| a (Å) | 22.1638(10) |
| b (Å) | 7.8126(3) |
| c (Å) | 11.9447(5) |
| α (°) | 90 |
| β (°) | 114.5200(10) |
| γ (°) | 90 |
| V (Å³) | 1881.78(14) |
| Z/Z′ | 4/1 |
| Temperature (K) | 100(2) |

12. Amorphous Free Form Compound II

### A. Synthetic Procedure

Amorphous free form Compound II was made by the process disclosed above in Example 3.

### B. X-Ray Powder Diffraction

X-ray powder diffraction spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step. The XRPD diffractogram for Amorphous free form Compound II is provided in FIG. 81.

### C. Solid-State NMR

The ¹³C CPMAS of Amorphous free form Compound II (FIG. 82) was acquired at 275 K with 12.5 kHz spinning and using adamantane as a reference. The peaks are listed in Table 72 below.

TABLE 72
Peak List from ¹³C CPMAS of Amorphous free form Compound II

| Peak # | Chem Shift [ppm] | Intensity [rel] |
|---|---|---|
| 1 | 150.2 | 21.7 |
| 2 | 142.8 | 15.8 |
| 3 | 136.1 | 13.9 |
| 4 | 126.1 | 36.7 |
| 5 | 74.3 | 57.4 |
| 6 | 65.6 | 32.1 |
| 7 | 63.0 | 40.4 |
| 8 | 48.2 | 100.0 |
| 9 | 43.8 | 28.5 |
| 10 | 37.2 | 42.7 |
| 11 | 22.4 | 33.1 |

### D. Thermogravimetric Analysis

Thermal gravimetric analysis of Amorphous free form Compound II was measured using TGA Q5000 from TA Instrument. A sample with weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, Del.). The thermogram showed 0.7% weight loss from ambient temperature up to 150° C. (FIG. 83).

### E. Differential Scanning Calorimetry Analysis

DSC of Amorphous free form Compound II was measured using the TA Instruments Discovery DSC 2500. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, Del.). The thermogram showed the glass transition occurred 78-88° C. (FIG. 84).

13. Compound II Phosphate Salt Acetone Solvate Form A

### A. Synthetic Procedure

Compound II Phosphate Salt Acetone Solvate Form A was made via mixing 351 mg of Amorphous free form Compound II and 20 mg of Compound II Phosphate Salt Hemihydrate Form A in a mixture of 3.5 ml acetone and 0.5 ml water. The sample was stirred at ambient temperature for 3 days before the solid was isolated for analysis.

Alternatively, acetone and water at a volume ratio of 0.984/0.016 was prepared, followed by adding Compound II Phosphate Salt Hemihydrate Form A to the solvent system at room temperature to form a suspension. After the sample was stirred overnight, it was filtered to obtain a clear saturated solution. An equal amount of Compound II Phosphate Salt Hemihydrate Form A and Compound II Phosphate Form C were added to the saturated solution. The sample was further stirred at ambient for 4 days until the solid was isolated for analysis.

### B. X-Ray Powder Diffraction

X-ray powder diffraction spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound II Phosphate Salt Acetone Solvate Form A is provided in FIG. 85 and the data is summarized below in Table 73.

TABLE 73
Peak list from XRPD diffractogram of Compound
II Phosphate Salt Acetone Solvate Form A

| No. | Pos. [±0.2, °2θ] | Rel. Int. [%] |
|---|---|---|
| 1 | 8.7 | 100.0 |
| 2 | 18.4 | 97.1 |
| 3 | 15.0 | 36.8 |
| 4 | 9.4 | 36.3 |
| 5 | 22.6 | 33.0 |
| 6 | 18.0 | 26.9 |
| 7 | 14.8 | 26.4 |
| 8 | 15.6 | 23.8 |
| 9 | 20.8 | 22.1 |
| 10 | 10.4 | 21.4 |
| 11 | 18.8 | 20.6 |
| 12 | 15.9 | 20.4 |
| 13 | 19.3 | 20.4 |
| 14 | 22.8 | 20.1 |
| 15 | 12.2 | 19.2 |
| 16 | 11.4 | 18.9 |
| 17 | 19.9 | 18.7 |
| 18 | 21.7 | 18.5 |
| 19 | 26.9 | 18.2 |
| 20 | 17.8 | 18.0 |
| 21 | 23.1 | 17.7 |
| 22 | 27.1 | 16.9 |
| 23 | 24.2 | 16.4 |
| 24 | 23.5 | 14.9 |
| 25 | 21.9 | 14.5 |
| 26 | 21.3 | 14.3 |
| 27 | 23.8 | 13.4 |
| 28 | 15.3 | 13.2 |
| 29 | 20.5 | 12.3 |
| 30 | 25.8 | 12.2 |
| 31 | 16.7 | 11.1 |
| 32 | 17.4 | 10.3 |
| 33 | 22.2 | 10.3 |

### C. Solid State NMR

The ¹³C CPMAS of Compound II Phosphate Salt Acetone Solvate Form A (FIG. 86) was acquired at 275 K with 12.5 kHz spinning and using adamantane as a reference. The peaks are listed in Table 74 below.

TABLE 74
Peak List from ¹³C CPMAS of Compound II
Phosphate Salt Acetone Solvate Form A

| Peak # | Chem Shift [ppm] | Intensity [rel] |
|---|---|---|
| 1 | 206.7 | 0.9 |
| 2 | 143.9 | 4.6 |
| 3 | 142.9 | 2.4 |
| 4 | 142.3 | 5.1 |
| 5 | 139.6 | 2.2 |
| 6 | 137.0 | 3.3 |
| 7 | 136.3 | 3.0 |
| 8 | 135.9 | 1.8 |
| 9 | 135.4 | 2.7 |
| 10 | 128.9 | 3.0 |
| 11 | 128.1 | 2.2 |
| 12 | 127.0 | 4.8 |
| 13 | 126.3 | 6.8 |
| 14 | 73.4 | 6.3 |
| 15 | 73.0 | 6.3 |
| 16 | 72.3 | 5.8 |
| 17 | 68.6 | 2.9 |
| 18 | 65.5 | 4.1 |
| 19 | 64.8 | 5.6 |
| 20 | 64.2 | 4.4 |
| 21 | 62.0 | 10.0 |
| 22 | 50.2 | 3.2 |
| 23 | 49.7 | 4.6 |
| 24 | 49.4 | 5.0 |
| 25 | 48.6 | 4.1 |
| 26 | 47.9 | 6.2 |
| 27 | 47.4 | 3.8 |
| 28 | 46.6 | 3.1 |
| 29 | 44.7 | 2.3 |
| 30 | 44.2 | 2.4 |
| 31 | 43.9 | 2.4 |
| 32 | 43.1 | 2.7 |
| 33 | 39.7 | 2.5 |
| 34 | 39.2 | 3.1 |
| 35 | 38.2 | 6.9 |
| 36 | 36.6 | 4.7 |
| 37 | 35.9 | 2.9 |
| 38 | 33.6 | 2.2 |
| 39 | 32.5 | 2.2 |
| 40 | 31.7 | 4.87 |
| 41 | 31.3 | 1.9 |
| 42 | 18.3 | 3.58 |
| 43 | 17.8 | 4.0 |
| 44 | 17.0 | 3.8 |
| 45 | 15.2 | 4.3 |

### D. Thermogravimetric Analysis

Thermal gravimetric analysis of Compound II Phosphate Salt Acetone Solvate Form A was measured using TGA Q5000 from TA Instrument. A sample with weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, Del.). The thermogram showed 0.9% weight loss from ambient temperature up to 200° C. (FIG. 87).

### E. Differential Scanning Calorimetry Analysis

DSC of Compound II Phosphate Salt Acetone Solvate Form A was measured using the TA Instruments Discovery DSC 2500. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, Del.). The thermogram showed an endothermic peak 242° C. (FIG. 88).

14. Compound II Phosphate Salt Form A

### A. Synthetic Procedure

Amorphous free form Compound II (30 mg) was weighed into a scintillation vial. To this was added to MEK (0.178 mL) followed by 0.178 mL of a 0.5 M stock solution of phosphoric acid (1.05 equivalents). 0.5 M phosphoric acid solution was prepared by first diluting 1.974 ml 15.2 M H₃PO₄ with 3.026 ml water to make 6 M stock, followed by diluting 0.417 ml of the 6 M stock with 4.583 ml MeOH.

The sample was stirred at ambient temperature. Precipitation started occurring after 24 hours. After 48 hours, the solids were filtered, washing with 4:1 n-Heptane/MEK (v/v). Subsequent washes were performed with n-Heptane resulting in a solid white powder. This sample was dried in a vacuum oven for 18 hours at 60° C.

### B. X-Ray Powder Diffraction

X-ray powder diffraction spectra were recorded at room temperature (25±2° C.) in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

The XRPD diffractogram for Compound II Phosphate Salt Form A is provided in FIG. 89 and the data is summarized below in Table 75.

TABLE 75
Peak list from XRPD diffractogram of
Compound II Phosphate Salt Form A

| No. | Pos. [±0.2, °2θ] | Rel. Int. [%] |
|---|---|---|
| 1 | 9.9 | 100.0 |
| 2 | 17.5 | 62.5 |
| 3 | 21.6 | 53.7 |
| 4 | 8.9 | 51.9 |
| 5 | 18.5 | 47.4 |
| 6 | 7.0 | 41.9 |
| 7 | 16.9 | 39.5 |
| 8 | 14.1 | 36.8 |
| 9 | 15.7 | 30.8 |
| 10 | 23.1 | 24.7 |
| 11 | 23.6 | 24.1 |
| 12 | 9.3 | 23.7 |
| 13 | 19.9 | 21.5 |
| 14 | 10.4 | 20.6 |
| 15 | 22.6 | 19.6 |
| 16 | 23.9 | 19.4 |
| 17 | 14.3 | 19.0 |
| 18 | 11.6 | 18.2 |
| 19 | 18.9 | 17.9 |
| 20 | 15.0 | 17.0 |
| 21 | 19.0 | 16.6 |
| 22 | 12.5 | 16.4 |
| 23 | 18.0 | 15.8 |
| 24 | 25.4 | 14.7 |
| 25 | 27.1 | 14.0 |
| 26 | 9.6 | 13.8 |
| 27 | 8.7 | 13.7 |
| 28 | 5.8 | 13.6 |
| 29 | 27.8 | 13.2 |
| 30 | 10.9 | 12.5 |
| 31 | 27.0 | 11.4 |
| 32 | 12.3 | 11.0 |
| 33 | 20.2 | 10.6 |
| 34 | 20.5 | 10.4 |

### C. Solid State NMR

The ¹³C CPMAS of Compound II Phosphate Salt Form A (FIG. 90) was acquired at 275 K with 12.5 kHz spinning and using adamantane as a reference. The peaks are listed in Table 76 below.

TABLE 76
Peak List from ¹³C CPMAS of Compound
II Phosphate Salt Form A

| Peak # | Chem Shift [ppm] | Intensity [rel] |
|---|---|---|
| 1 | 144.1 | 23.6 |
| 2 | 143.3 | 27.3 |
| 3 | 142.5 | 41.6 |
| 4 | 141.4 | 35.4 |
| 5 | 139.9 | 12.2 |
| 6 | 136.4 | 45.6 |
| 7 | 129.1 | 27.0 |
| 8 | 126.6 | 55.5 |
| 9 | 125.9 | 55.2 |
| 10 | 72.9 | 100.0 |
| 11 | 72.1 | 73.9 |
| 12 | 68.5 | 14.3 |
| 13 | 65.4 | 49.0 |
| 14 | 64.4 | 64.0 |
| 15 | 64.1 | 73.5 |
| 16 | 62.0 | 78.1 |
| 17 | 49.4 | 80.1 |
| 18 | 48.4 | 49.9 |
| 19 | 47.6 | 56.8 |
| 20 | 46.5 | 42.9 |
| 21 | 44.2 | 26.3 |
| 22 | 43.1 | 37.5 |
| 23 | 39.7 | 28.7 |
| 24 | 38.4 | 55.0 |
| 25 | 37.4 | 41.9 |
| 26 | 36.2 | 47.2 |
| 27 | 34.4 | 8.2 |
| 28 | 33.0 | 24.1 |
| 29 | 30.6 | 8.7 |
| 30 | 17.5 | 63.8 |
| 31 | 16.2 | 29.7 |
| 32 | 14.9 | 45.9 |
| 33 | 9.3 | 12.4 |

The ³¹P CPMAS of Compound II Phosphate Salt Form A (FIG. 91 and Table 77) was acquired at 275 K with 12.5 kHz spinning and using adamantane as a reference.

TABLE 77
Peak list from ³¹P CPMAS of Compound II Phosphate Salt Form A

| Peak # | Chem Shift [ppm] | Intensity [rel] |
|---|---|---|
| 1 | 3.3 | 100.0 |
| 2 | 2.2 | 70.3 |
| 3 | −0.4 | 37.5 |

### D. Thermogravimetric Analysis

Thermal gravimetric analysis of Compound II Phosphate Salt Form A was measured using TGA Q5000 from TA Instrument. A sample with weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, Del.). The thermogram showed negligible weight loss from ambient temperature up to 200° C. (FIG. 92).

### E. Differential Scanning Calorimetry Analysis

DSC of Compound II Phosphate Salt Form A was measured using the TA Instruments DSC Q2000. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 2° C./min (modulated temperature amplitude at 0.3200° C., period 60 seconds) to 300° C. When the run was completed, the data were analyzed by Trios and/or Universal Analysis software (TA Instruments, New Castle, Del.). The thermogram showed endothermic peaks around 228° C. and 237° C. (FIG. 93).

15. Compound II Phosphate Salt Form C

### A. Synthetic Procedure

Compound II Phosphate Salt Form C was obtained from a slurry of Compound II Phosphate Salt Hemihydrate Form A in 1-butanol at 80° C.

### B. X-Ray Powder Diffraction

XRPD was performed with a Panalytical X'Pert³ Powder XRPD on a Si zero-background holder. The 20 position was calibrated against a Panalytical Si reference standard disc. The parameters used are listed in the following table.

TABLE
Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2θ) | 3-40 |
| Scan step time [s] | 18.87 |
| Step size (°2θ) | 0.0131 |
| Test Time | 4 min 15 s |

The XRPD diffractogram for Compound II Phosphate Salt Form C is provided in FIG. 94 and the data is summarized below in Table 78.

TABLE 78
Peak list from XRPD diffractogram of
Compound II Phosphate Salt Form C

| No. | Pos. [±0.2, °2θ] | Rel. Int. [%] |
|---|---|---|
| 1 | 9.1 | 100.0 |
| 2 | 15.0 | 76.8 |
| 3 | 11.0 | 55.5 |
| 4 | 9.4 | 46.8 |
| 5 | 10.4 | 46.1 |
| 6 | 18.6 | 44.4 |
| 7 | 18.3 | 39.6 |
| 8 | 21.4 | 38.8 |
| 9 | 13.5 | 32.3 |
| 10 | 20.9 | 25.6 |
| 11 | 21.2 | 23.1 |
| 12 | 15.5 | 22.4 |
| 13 | 20.7 | 21.8 |
| 14 | 18.8 | 20.8 |
| 15 | 22.6 | 18.6 |
| 16 | 24.3 | 18.5 |
| 17 | 13.7 | 17.0 |
| 18 | 27.5 | 15.7 |
| 19 | 23.2 | 14.3 |
| 20 | 27.3 | 14.1 |
| 21 | 16.5 | 13.9 |
| 22 | 21.7 | 13.4 |
| 23 | 26.5 | 11.2 |
| 24 | 22.8 | 11.1 |

### C. Solid State NMR

The ¹³C CPMAS of Compound II Phosphate Salt Form C (FIG. 95) was acquired at 275 K with 12.5 kHz spinning and using adamantane as a reference. The peaks are listed in Table 79 below.

TABLE 79
Peak List from ¹³C CPMAS of Compound
II Phosphate Salt Form C

| Peak # | Chem Shift [ppm] | Intensity [rel] |
|---|---|---|
| 1 | 143.0 | 8.4 |
| 2 | 140.3 | 9.5 |
| 3 | 139.6 | 6.6 |
| 4 | 139.0 | 5.5 |
| 5 | 129.2 | 3.3 |
| 6 | 127.8 | 4.5 |
| 7 | 127.0 | 3.3 |
| 8 | 125.5 | 4.1 |
| 9 | 124.6 | 3.7 |
| 10 | 73.0 | 10.0 |
| 11 | 72.7 | 9.7 |
| 12 | 66.5 | 5.6 |
| 13 | 64.1 | 4.3 |
| 14 | 62.5 | 4.5 |
| 15 | 50.4 | 3.9 |
| 16 | 47.7 | 6.0 |
| 17 | 45.2 | 3.5 |
| 18 | 43.5 | 4.9 |
| 19 | 39.6 | 3.2 |
| 20 | 39.0 | 3.4 |
| 21 | 38.2 | 9.0 |
| 22 | 16.8 | 6.7 |
| 23 | 16.2 | 6.6 |

### D. Thermogravimetric Analysis

Thermal gravimetric analysis of Compound II Phosphate Salt Form C was measured using TGA Q5000 from TA Instrument. A sample with weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, Del.). The thermogram showed about 1.6% weight loss from ambient temperature up to 150° C. (FIG. 96).

### E. Differential Scanning Calorimetry Analysis

DSC of Compound II Phosphate Salt Form C was measured using the TA Instruments DSC Q2000. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 2° C./min (modulated temperature amplitude at 0.3200° C., period 60 seconds) to 300° C. When the run was completed, the data were analyzed by Trios and/or Universal Analysis software (TA Instruments, New Castle, Del.). The thermogram showed an endothermic peak around 244° C. (FIG. 97).

### Example 5: Alternative Syntheses of Compound I and Compound II

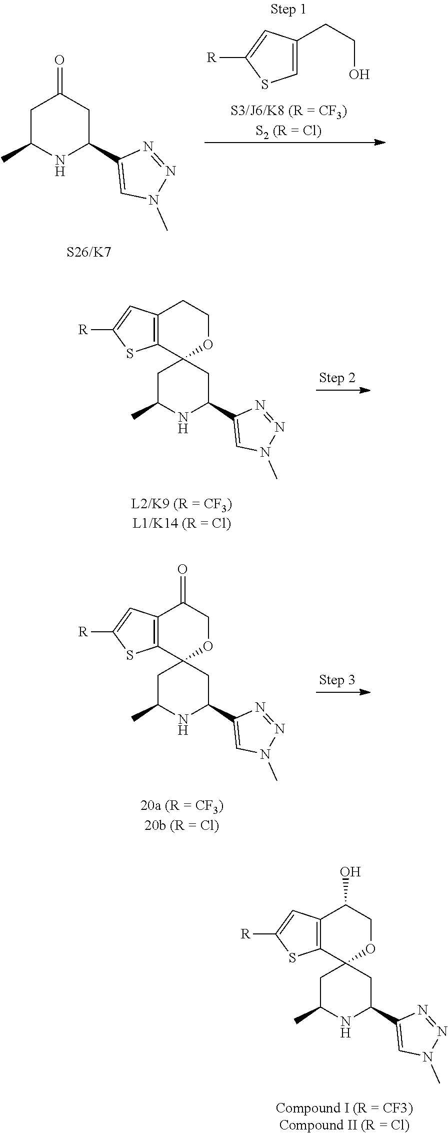

Step 1 (Compound L2/K9): A solution of Compound S26/K7 (70 g, 0.360 mol, 1.0 equiv) and 2-[5-trifluoromethyl)-3-thienyl]ethanol (Compound S3/J6/K8) (74.2 g, 0.378 mol, 1.05 equiv) in DCM (210 mL, 3 vol) was cooled to 5° C. Methanesulfonic acid (210.6 mL, 3.24 mol, 9 equiv) was charged to the reactor while maintaining an internal temperature <30° C. The resulting reaction mixture was heated to 39° C. After 18 hours, HPLC analysis indicated >99% conversion to Compound L2/K9. The reaction mixture was cooled to 30° C., charged with DCM (280 mL, 4 vol) and further cooled to 0° C. The pH was adjusted to pH 10 with 4 M sodium hydroxide (830 mL). The organic layer was separated, and the aqueous phase was back extracted with DCM (350 mL, 5 vol). The combined organics were washed with water (350 mL, 5 vol) and concentrated at reduced pressure to 3.5 total volumes. The mixture was charged with MTBE (350 mL, 5 vol) and concentrated under reduced pressure to 3.5 total volumes. This put/take cycle was repeated three additional times and the resulting 3.5 vol mixture was diluted with MTBE (455 mL, 6.5 vol) to provide a 10 vol mixture. The slurry was heated to 50° C., stirred for 5 h, then charged with n-heptane (700 mL, 10 vol) over 2 h. The resulting suspension was cooled to 20° C. over 5 h and stirred for 18 h. The suspension was filtered, washed with 1:2 MTBE/n-heptane (2×140 mL, 2×2 vol) and dried under vacuum while flushing with nitrogen at 50° C. for 18 hours to give 103 g of Compound L2/K9 (77% yield).

Step 1 (Compound L1/K14): To a 20 L jacketed reactor was charged Compound S26/K7 (500.0 g, 2.58 mol, 1.0 equiv), DCM (3000 mL, 6 vol) and Compound S2 (440.1 g, 2.71 mol, 1.05 equiv). The equipment was rinsed with additional DCM (500 mL, 1 vol), which was charged to the reactor. The mixture was cooled to 0° C. and methanesulfonic acid (1734 g, 18.04 mol, 7.0 equiv) was added over 2.5 h while maintaining an internal temperature below 15° C. The mixture was heated to 38° C. and stirred at that temperature for 16 hours at which time HPLC analysis indicated complete conversion to Compound L1/K14. The mixture was cooled to 0° C. and the pH was adjusted to pH 10 with the addition of 4 M sodium hydroxide (3000 mL, 6 vol). The organic layer was separated and washed with water (2000 mL, 4.0 vol). The resulting organic phase was concentrated under reduced pressure to 3.5 volumes at 30° C. MTBE (5000 mL, 10 vol) was charged and the mixture was concentrated to 7.0 volumes under reduced pressure at 30° C. This put/take cycle was repeated two additional times. The resulting suspension was diluted with MTBE (1000 mL, 2 vol) to give a suspension of 9 total volumes. The suspension was heated to 50° C. for 2 hours, then charged with n-heptane (4500 mL, 9 vol) over 2 hours. The suspension was stirred at 50° C. for 12 h then cooled to 20° C. over 3 hours. After stirring at 20° C. for an additional 2 hours, the slurry was filtered, washed with 1:1 MTBE/n-heptane (1000 mL, 2.0 vol) and dried at 50° C. under vacuum with nitrogen stream for 16 hours to afford 700 g of Compound L1/K14 (92% yield). A recrystallization was performed by suspending Compound L1/K14 (8.2 kg) in a mixture of MTBE (41 L, 5 vol) and DCM (12 L, 1.5 vol) and heating the suspension to 55° C. n-Heptane (16 L, 2.0 vol) was added over 2 h. Compound L1/K14 (0.05 wt %) seed crystals were charged and the resulting slurry was stirred for 1 h at 50° C. n-Heptane (25 L, 3.0 vol) was charged over 3 h and the mixture was stirred at 50° C. for an additional 1 hour. The mixture was cooled to 20° C. over 4 hours and stirred at that temperature for 16 hours. The solids were filtered washed with 1:1 MTBE/n-heptane (16 L, 2 vol), and dried under vacuum with nitrogen bleed at 50° C. for 18 h to afford 6632 g of Compound L1/K14. (81% yield from 8.2 kg of Compound L1/K14).

Step 2. Method A (Compound 20a): In a holding vessel were dissolved Compound L2/K9 (4.5 g, 12.1 mmol), 2,4,6-triphenylpyrylium tetrafluoroborate (48 mg, 0.12 mmol, 1 mol %), and MsOH (0.94 mL, 14.5 mmol, 1.2 equiv) in MeCN (36 mL, 8 V) at 0° C. The reaction mixture was then flowed through a flow recirculation loop (⅛″ inch inner diameter tubing, 10 mL, 0.26 min residence time), passing through an inline gas/liquid mixer with 80 standard cubic centimeter/min of a 1:1 mixture of dry air and N2 before irradiation with 460 nm LEDs at 20° C. During recirculation, catalyst was continuously added to the holding vessel at a rate of 6 mol %·h⁻¹ (total catalyst charge of 13 mol %). After 2 hours, catalyst addition was stopped, and the reaction mixture was allowed to continue recirculating. After 15 minutes, irradiation was stopped and HPLC indicated 60% assay yield. The reaction mixture was diluted with water (18 mL, 4 V) and distilled under reduced pressure to a total of 4 volumes. 2-MeTHF (36 mL, 8 V) was added, and the internal temperature of the mixture was adjusted to 10° C. The pH of the aqueous phase was adjusted to 6-7 using NaOH (2 M, 9.1 mL, 18.2 mmol, 1.5 equiv), then to 9-10 using Na₂CO₃ (1 M, 9.1 mL, 9.1 mmol, 0.75 equiv). The mixture was then warmed to 20° C. with stirring for 30 min, and then stirring was stopped, allowing the phases to settle for no less than 30 min. The aqueous layer was removed, and the organic phase was distilled under reduced pressure to 4.0 volumes. 2-MeTHF was exchanged for IPA by thrice diluting with IPA (36 mL, 8 V) and distilling under reduced pressure to a total of 4 volumes. The internal temperature of the IPA solution was adjusted to 50° C. and agitated for no less than 30 min. MsOH (0.82 mL, 12.7 mmol, 1.05 equiv) was added over 30 min at 50° C. and the solution was cooled to 20° C. over 6 h. The resulting slurry was stirred at 20° C. for 15 h and the batch was then vacuum filtered. The filter cake was rinsed twice with IPA (4.5 mL, 1 V) then dried in a vacuum oven at 50° C. for 18 h until constant weight was achieved, affording Compound 20a (2.4 g, 41%).

### Step 2. Method B (Compound 20a): Compound L1/K14

(1 g, 1 equiv) was combined with cupric acetate (0.146 g, 0.806 mmol, 0.3 eq), diluted with acetonitrile (5.00 mL, 5 vol) and water (5.00 mL, 5 vol), and stirred under nitrogen until a clear blue solution formed. A solution of ammonium persulfate (2.14 g, 9.40 mmol, 3.5 eq) in water (10.0 mL, 555 mmol, 10 vol) was prepared separately and added dropwise to the solution of Compound L1/K14. The resulting solution was heated to 50° C. under nitrogen and stirred overnight. The reaction solution was then cooled to 20° C. and charged with isopropyl acetate (10 mL, 10 vol) and 30% ammonium hydroxide solution (10 mL, 10 vol). Additional isopropyl acetate was charged and the phases were mixed then separated. The organic phase was washed with a mixture of 30% commercial ammonium hydroxide solution (3×10 mL, 3×10 vol) and saturated ammonium chloride solution (1 mL, 1 vol). The combined aqueous washes were back-extracted with isopropyl acetate (2×10 mL, 2×10 vol). The organic phases were combined, dried over Na₂SO₄, filtered, and concentrated. The resulting residue was concentrated from MeCN and then from DCM to give a foam, which was dissolved in DMF (2 mL) and purified by reverse and normal phase chromatography to afford Compound 20a as a TFA salt (440 mg) in 33% yield. The free base of Compound 20a can be obtained by taking up the Compound 20a TFA salt in DCM, washing with 1 M NaOH (aq) and back-extracting the aqueous phase with additional DCM. The combined organic phase was then washed with brine, dried over Na₂SO₄, concentrated, and dried under vacuum to afford Compound 20a.

Step 2. Method A (Compound 20b): In a holding vessel were dissolved Compound L1/K14 (4.5 g, 13.3 mmol), 2,4,6-triphenylpyrylium tetrafluoroborate (52.6 mg, 0.13 mmol, 1 mol %), and methanesulfonic acid (MsOH, 1.04 mL, 16.0 mmol, 1.2 equiv) in MeCN (36 mL, 8 V) at 0° C. The reaction mixture was then flowed through a flow recirculation loop (⅛ inch inner diameter tubing, 10 mL, 0.26 min residence time), passing through an inline gas/liquid mixer with 80 standard cubic centimeter/min of a 1:1 mixture of dry air and N2 before irradiation with 460 nm LEDs at 20° C. During recirculation, catalyst was continuously added to the holding vessel at a rate of 6 mol %.11⁻¹ (total catalyst charge of 13 mol %). After 2 hours, catalyst addition was stopped, and the reaction mixture was allowed to continue recirculating. After 15 minutes, irradiation was stopped and HPLC indicated 30% assay yield. An aqueous work-up is carried out and the desired product is isolated.

Step 2. Method B (Compound 20b): Compound 20b is prepared according to a procedure analogous to that for Example 5, Step 2 Method B (Compound 20a).

Step 2. Method C (Compound 20b): In a holding vessel were dissolved Compound 1b (1.0 g, 3.0 mmol), 2,4,6-triphenylpyrylium tetrafluoroborate (11.7 mg, 0.03 mmol, 1 mol %), and methanesulfonic acid (MsOH, 0.23 mL, 3.5 mmol, 1.2 equiv) in AcOH (28.8 mL, 28.8 V) at 20° C. The reaction mixture was then flowed through a flow recirculation loop (2 mL internal volume, 15 mL/min liquid flow rate), passing through an inline gas/liquid mixer with a 1:1 mixture of dry air and N2 (7.5 mL/min gas flow rate) before irradiation with 450 nm LEDs at 20° C. During recirculation, a solution of 2,4,6-triphenylpyrylium tetrafluoroborate (140.3 mg, 0.35 mmol, 12 mol %) in MeCN (3.2 mL, 3.2 V) was continuously added to the holding vessel over 30 min (24 mol %·h⁻¹). After a total of 45 min recirculation, irradiation was stopped and qNMR indicated 65% assay yield. Workup and isolation procedures for Compound 20b are expected to closely mirror those of Compound 20a.

Step 3. Method A (Compound I): A 1:1 triethylamine/formic acid solution was prepared by combining triethylamine (356 uL, 2.56 mmol) and formic acid (96.5 uL, 2.56 mmol). The resulting mixture was treated with a prepared solution of (R,R)-TsDPEN (1.12 mg, 0.00307 mmol, 0.003 equiv) and pentamethylcyclopentadienylrhodium(III)chloride dimer (0.62 mg, 0.001 mmol, 0.001 equiv) in DCM (1 vol, 0.40 mL) and stirred under nitrogen. In a separate flask, a solution of Compound 20a (401 mg, 1.02 mmol) in DCM (4 mL, 10 vol) was prepared. The solution of Compound 20a in DCM was then added dropwise to the catalyst mixture over several minutes at 20° C. The resulting mixture was stirred overnight at 20° C., after which time HPLC analysis indicated 97% conversion to Compound I. The mixture was diluted with DCM (0.8 mL, 2 vol) and washed with saturated, aqueous NaHCO₃ (2 mL, 5 vol). The resulting aqueous phase was back extracted with DCM (0.8 mL, 2 vol) and the combined organics were washed with water (2 mL, 5 vol). The resulting aqueous phase was back extracted with DCM (1.6 mL, 4 vol), and the combined organics were washed with saturated, aqueous NaCl (1.6 mL, 4 vol). The resulting organic phase was concentrated to dryness to afford Compound I.

Step 3. Method B (Compound I): To a 100 mL reactor was charged Compound 20a (5.00 g, 12.9 mmol, 1 equiv), (pentamethylcyclopentadienyl)rhodium(III) dichloride dimer (4.0 mg, 0.0065 mmol, 0.0005 equiv), and (1R,2R)-(−)-N-(4-toluenesulfonyl)-1,2-diphenylethylenediamine (5.7 mg, 0.015 mmol, 0.0012 equiv). After flushing the head space with nitrogen for 5 minutes, acetonitrile (21.5 mL, 4.3 vol) was charged and agitation (200 rpm) was started. The solution was stirred at 20° C. for 30 minutes then cooled to 5° C. over 30 minutes. To a separate 20 mL vial were charged acetonitrile (3.5 mL, 0.7 vol) and triethylamine (2.16 mL, 15.5 mmol, 1.2 equiv) and the resulting solution was stirred in an ice water bath for 5 minutes. Formic acid (0.54 mL, 14.2 mmol, 1.1 equiv) was then charged over 5 minutes. The formic acid/triethylamine solution was charged to the solution of substrate via syringe over 3 hours. The resulting solution was stirred at 5° C. for 19 hours, at which time >99.5% conversion to Compound I was observed by HPLC. The suspension was heated to 55° C. to give a homogeneous solution, which was concentrated under reduced pressure to 4.0 volumes at 55° C. Acetonitrile (15 mL, 3 vol) was charged and the solution was again concentrated under reduced pressure to 4.0 volumes at 55° C. The solution was cooled to 52° C. over 10 minutes the seed crystals of Compound I (25 mg, 0.05 wt % with respect to Compound 20a) were added. The suspension was held at 52° C. for 1 hour, cooled to 20° C. over 3 hours and held at 20° C. for 18 hours. The suspension was filtered and the solids washed with acetonitrile (3×3 mL, 1.8 vol). The solid was dried on filter funnel for 10 minutes and further dried in vacuum oven at 50° C. for 16 hours to provide 3.78 g of Compound I (74% yield).

Step 3 (Compound II): Compound II is prepared according to a procedure analogous to that for Example 5, Step 3 Method B (Compound I).

### Example 6: Alternative Synthesis of Compound I

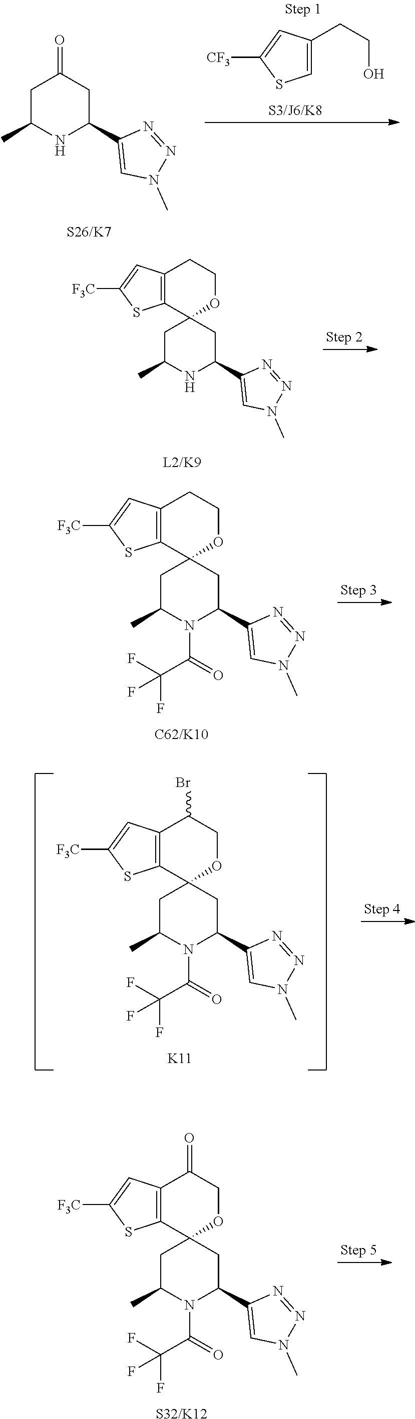

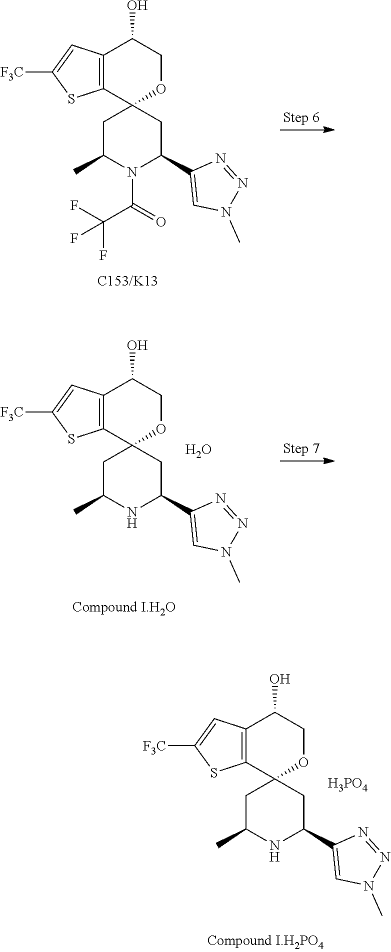

Step 1: A solution of S26/K7 (70 g, 0.360 mol, 1.0 equiv) and 2-[5-trifluoromethyl)-3-thienyl]ethanol (S3/J6/K8) (74.2 g, 0.378 mol, 1.05 equiv) in DCM (210 mL, 3 vol) was cooled to 5° C. Methanesulfonic acid (210.6 mL, 3.24 mol, 9 equiv) was charged to the reactor while maintaining an internal temperature <30° C. Optionally, other organic or mineral acids may be used for this step. The resulting reaction mixture was heated to 39° C. After 18 hours, HPLC analysis indicated >99% conversion to L2/K9. The reaction mixture was cooled to 30° C., charged with DCM (280 mL, 4 vol) and further cooled to 0° C. The pH was adjusted to pH 10 with 4 N sodium hydroxide (830 mL). The organic layer was separated, and the aqueous phase was back extracted with DCM (350 mL, 5 vol). The combined organics were washed with water (350 mL, 5 vol) and concentrated at reduced pressure to 3.5 total volumes. The mixture was charged with MTBE (350 mL, 5 vol) and concentrated under reduced pressure to 3.5 total volumes. This put/take cycle was repeated three additional times and the resulting 3.5 vol mixture was diluted with MTBE (455 mL, 6.5 vol) to provide a 10 vol mixture. The slurry was heated to 50° C., stirred for 5 h, then charged with n-heptane (700 mL, 10 vol) over 2 h. The resulting suspension was cooled to 20° C. over 5 h and stirred for 18 h. The suspension was filtered, washed with 1:2 MTBE/n-heptane (2×140 mL, 2×2 vol) and dried under vacuum while flushing with nitrogen at 50° C. for 18 hours to give 103 g of L2/K9 (77% yield).

Step 2: A solution of L2/K9 (50 g, 0.134 mol, 1.0 equiv) and triethylamine (22.5 mL, 0.161 mol, 1.2 equiv) in DCM (380 mL, 7.6 vol) was cooled to 5° C. Alternatively, other amine bases may be employed for this step. At 5° C., trifluoroacetic acid anhydride (20.5 mL, 0.148 mol, 1.1 equiv) was charged to the reactor while keeping the internal temperature below 15° C. The resulting reaction mixture was stirred at 5° C. for 1 h at which time HPLC showed 99.8% conversion to C62/K10. The reaction mixture was charged at 5° C. with water (200 mL, 4 vol). The organic layer was separated and sequentially washed with 5% NaHCO₃ (200 mL, 4 vol), 2N HCl (2×200 mL, 2×4 vol) and water (2×200 mL, 2×4 vol). The organic layer was concentrated under reduced pressure to 3.5 total volumes. MTBE (400 mL, 8 vol) was charged and the batch was concentrated under reduced pressure to 3.5 vol. This put/take cycle was repeated two additional times and the mixture was concentrated to 3 volumes after the final cycle. The solution was heated to 40° C., charged with n-heptane (190 mL, 2 vol) over 1 h, then cooled to 20° C. over 2 h to yield a suspension. n-Heptane (500 mL, 10 vol) was charged over 2 h and the resulting suspension was stirred for 18 h. The suspension was filtered, washed with 5% MTBE/n-heptane (2×125 mL, 2×2.5 vol) and dried under vacuum while flushing with nitrogen at 50° C. for 18 hours to give 53 g of C62/K10 (84% yield).

Steps 3 and 4: C62/K10 (20.0 g, 42.7 mmol, 1 equiv) and 1,3-dibromo-5,5-dimethylhydantoin (8.54 g, 29.9 mmol, 0.70 eq) were combined in a reactor and diluted with chlorobenzene (80.0 mL, 787 mmol, 4 vol). The resultant slurry was sparged sub-surface with nitrogen for 15 min. Alternatively, other brominating agents, such as, e.g., NBS, may be used for this step. The slurry was then heated to 50° C. over 30 minutes. In a separate flask, a solution of the 2,2′-azo-bis-isobutyronitrile (0.561 g, 3.42 mmol, 0.08 eq) in chlorobenzene (20.0 mL, 197 mmol, 1 vol) was prepared and added to the reactor containing the C62/K10 solution over 1 hour. The resulting mixture was stirred at 50° C., under N₂, overnight. The reaction solution was sparged sub-surface with N₂ for 15 minutes, then anhydrous dimethyl sulfoxide (100 mL, 1410 mmol, 5 vol) was added followed by anhydrous triethylamine (29.8 mL, 213 mmol, 5 eq). Optionally, other amine bases may be used to affect this transformation. The solution was sparged subsurface with N₂ for 30 minutes then heated to 70° C. and stirred overnight. The reaction was cooled to 5° C. and diluted with DCM (40 mL, 2 vol). Water (60 mL, 3 vol) was added followed by DCM (20 mL, 1 vol). The phases were mixed and then separated. The aqueous phase was extracted with dichloromethane (60 mL, 3 vol) and the combined organics were washed sequentially with 2N HCl (100 mL, 5 vol) and water (2×100 mL, 2×5 vol). The organic phase was concentrated under reduced pressure to 3 total volumes. The solution was charged with IPA (160 mL, 8 vol) and concentrated under reduced pressure to 3 volumes. This put/take cycle was repeated two additional times giving a 3-volume solution that was further diluted with IPA (20 mL, 1 vol). The resulting 4 vol mixture was heated to 75° C. to provide a homogenous solution and cooled to 50° C. The solution was seeded with S32/K12 (0.05 wt %) at 50° C., stirred for 1 h and further cooled to 20° C. over 2 h. After stirring an additional 18 h at 20° C., the slurry was charged with n-heptane (20 mL, 1 vol) over 1 h. The slurry was stirred for 4 h at 20° C., filtered, washed with 1:1 IPA/n-heptane (2×20 mL, 2×1 vol) and dried under vacuum while flushing with nitrogen at 50° C. for 18 hours to give S32/K12 (46% yield from C62/K10). The dried S32/K12 (31.0 g) was suspended in IPA (93 mL, 3 vol), heated to 80° C. and stirred at that temperature for 2 h. The solution was cooled to 70° C. over 1 h and stirred for 1 h. The suspension was cooled to 20° C. over 5 hours and stirred at that temperature for 18 h. The suspension was filtered, washed with 1:1 IPA/n-heptane (2×35 mL, 2×0.5 vol) and dried under vacuum while flushing with nitrogen at 50° C. for 18 hours to give 28.8 g S32/K12 (93% yield from S32/K12).

Step 5: To a 400 L jacketed Hastelloy reactor was charged S32/K12 (15.01 kg, 31.11 mol), pentamethylcyclopentadienyl rhodium chloride dimer (RhCl₂Cp)₂ (10.37 g, 0.016 mol, 0.0005 eq), (R,R)—N-(p-toluenesulfonyl)-1,2-diphenylethylenediamine (R,R-TsDPEN, 12.0 g, 0.032 mol, 0.001 eq) and ACN (105 L, 7 vol). The resulting solution was stirred while cooling to −20° C. Once at temperature, (1:1) formic acid/triethylamine (25 L, 11.4 kg, 77.6 mol, 2.5 eq) in ACN (15 L, 1 vol) was slowly added over a 2 hour period. The resulting solution was stirred at −20° C. for 8 hours before slowly warming to −10° C. and holding for 1 hour. The reaction was then slowly warmed to 0° C. and held for 3 hours before warming to 20° C. and stirring overnight. When HPLC analysis indicated the reaction was complete, MTBE (90 L, 6 vol) was charged followed by 10% NaCl (75 L, 5 vol). The resulting mixture was stirred vigorously for 30 minutes before allowing the phases to separate. The organic layer was washed with 0.5 N HCl (75 L, 5 vol) and the resulting aqueous layer was back extracted with MTBE (20 L, 1.2 vol). The combined organics were washed with 10% NaCl (75 L, 5 vol) and concentrated at reduced pressure to 50 L (3.3 vol). MTBE (120 L, 8 vol.) was charged and the resulting solution was again concentrated at reduced pressure to 50 L (3.3 vol.). This put/take cycle was repeated four additional times and the resulting organic layer was diluted with DCM (50 L, 3.3 vol). Florisil (8 kg, 50 wt %) was charged and the resulting slurry was stirred at 20° C. for 2.5 hours before filtering off the solid. The isolated solids were rinsed twice with 2:1 DCM/MTBE (2×16 L, 2×1 vol). To the combined filtrate and washes was added Florisil (8 kg, 50 wt %) and the resulting slurry was stirred at 20° C. overnight. The solids were again filtered and rinsed with 2:1 DCM/MTBE (2×16 L, 2×1 vol). The combined organics were concentrated at reduced pressure to 50 L (3 vol). MTBE (120 L, 8 vol.) was added and the resulting solution was again concentrated at reduced pressure 50 L (3.3 vol.). This put/take cycle was repeated three additional times and the resulting mixture was diluted to 5 total volumes with MTBE. The slurry was stirred and heated to 50° C. and held at that temperature for 1 hour before adding n-heptane (64 L, 4 vol) over 1 hour. The resulting slurry was stirred at 50° C. for one hour then cooled to 20° C. over 2 hours. The slurry was stirred overnight at 20° C. then filtered. The solid was rinsed with 1:1 IPA/n-heptane (32 L, 2 vol). The solid was dried under a heated nitrogen sparge with additional vacuum for 48 hours to afford Compound C153/K13 in 88% yield.

Step 6. Method A: C153/K13 (43.5 g, 89 mmol, 1 equiv) and methanol (150.0 mL, 3 vol) were combined and agitated until full dissolution was observed. 6 N NaOH (89 mL, 6 eq) was added over 30 minutes and the mixture was heated to 60° C. After stirring at 60 C for 1 hour, HPLC indicated complete conversion to Compound I. Optionally, other metal hydroxides like LiOH, KOH or CsOH could be used for this step. The reaction solution was cooled to 15° C. and treated with isopropyl acetate (250 mL, 5.75 vol). Water (100 mL, 2.3 vol) was then added and the mixture was agitated for 30 minutes. The phases were separated, and the aqueous phase was back extracted with isopropyl acetate (250 mL, 5.75 vol). The organics were combined and washed with 10% NaCl (aq) (2×250 mL, 2×5.75 vol) and water (250 mL, 5.75 vol). The organics were concentrated under reduced pressure to 4.0 total volumes (174 mL). The solution was charged with MTBE (500 mL, 11.5 vol) and concentrated again to 4.0 vol. This put/take cycle was repeated three additional times. MTBE (75 mL, 1.75 vol) was added to give a 5.75 total volume solution. While stirring at 20° C., water (3.2 mL, 180 mmol, 2 eq) was added over 2 hours, inducing crystallization. The slurry was stirred at 20° C. for 1 hour then heated to 50° C. and stirred at that temperature for 3 hours. The suspension was cooled to 20° C. and stirred for 18 hours at that temperature. The slurry was filtered under vacuum and the cake was washed with MTBE (100 mL, 2.3 vol). The solids were dried at 50° C. under vacuum for 18 hours to provide 29 g of Compound I.H₂O (81% yield).

Step 6. Method B: C153/K13 (25.00 g, 42.27 mmol; 81.9% potency (20.48 g used for volume calculations)) was charged to a 3-neck 500 mL round bottom flask. Ethanol (46.1 mL, 2.25 vol) was added, followed by the 3 M NaOH (28.2 mL, 2 equiv) to give a slurry. The slurry was heated to 50° C. and stirred 2 h when HPLC showed complete conversion to Compound I. Water (81.9 mL, 4 vol) was added over 1 hour to the reaction solution at 50° C., maintaining a solution. The resultant solution was seeded with Compound I seed crystals (0.16 g). The seeded solution stirred 1 hour at 50° C. with the seeds holding and minimal additional crystallization occurring. Additional water (165.9 mL, 8.1 vol) was added over 1 hour to give a slurry. The slurry was cooled to 20° C. over 1 hour and stirred at 20° C. overnight. The resultant slurry was filtered under vacuum filtration. The reaction flask was rinsed with a pre-mixed solution of water:EtOH (6:1 v/v, 25 mL, 1.2 vol) and the rinse added to the solids in the frit and filtered. The solids were rinsed three more times with a pre-mixed solution of water:EtOH (6:1 v/v, 3×25 mL, 3×1.2 vol). The solid Compound I was dried further under vacuum filtration to provide 16.4 g of Compound I (91.7%).

Step 7 (Compound I.H₃PO₄): Compound I.H₂O (50.02 g, 119.627 mmol, 1 equiv) was charged to a 500 mL jacketed reactor equipped with a retreat curve mechanical stirrer, a huber ministat, a findenser, and an N2 bubbler. MEK (300 mL, 6 vol) and water (10.5 mL, 0.2 vol) were added to the reactor and the mixture was stirred at 20° C. In a separate vessel, phosphoric acid (14.067 g, 85 w/w %, 122.02 mmol, 1.02 equiv) was mixed with MEK (190 mL, 3.8 vol). To the clear solution in reactor, Compound I.H₃PO₄ (0.582 g, 1.196 mmol, 0.01 equiv.) was added as seeds. A portion of 10 mL (0.2 vol) of the prepared phosphoric acid solution was also added to the reactor and the resulting slurry was stirred for 1 hour. The remaining phosphoric acid solution (188 mL) was added at a linear rate over 12 hours. The slurry was left to agitate at 20° C. overnight and was then filtered. The wet cake was washed with 2% by volume water in MEK (150 mL, 0.797 M, 3 vol). The solids were transferred to drying dish and placed in a vacuum oven at 80° C. under a nitrogen bleed and dried for 24 hours to yield 58.63 g of Compound I.H₃PO₄ (97% yield). As required, Compound I.H₃PO₄ (2718.7 g) was reprocessed by suspending in a 1:1 mixture of MEK/methanol (13.6 L, 5 vol). The suspension was heated to 50° C. and stirred at that temperature for 3 hours. The suspension was then cooled to 20° C. over 1 h and stirred for 30 minutes. The resulting solids were filtered, washed with n-heptane (13.6 L×2,5 vol×2) and dried at 80° C. under vacuum with nitrogen bleed to afford 2641.9 g of Compound I.H₃PO₄ (97% yield from 2718.7 g of Compound I.H₃PO₄).

### Example 7: Alternative Syntheses of Compound II

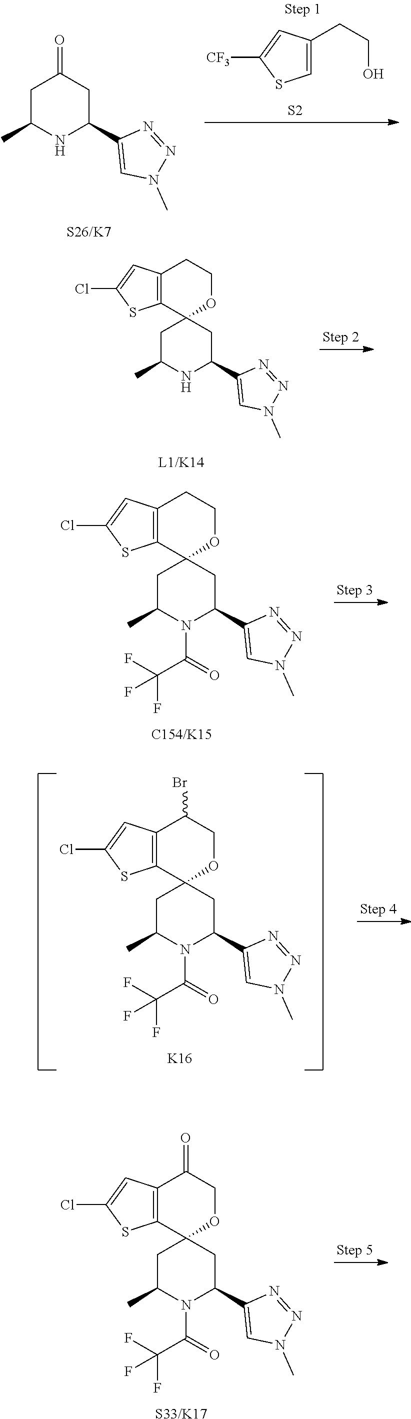

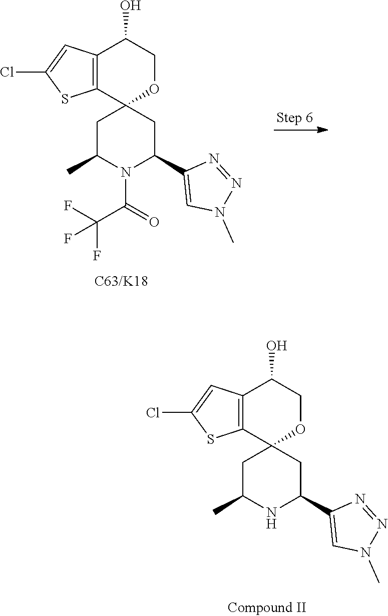

Step 1: To a 20 L jacketed reactor was charged 526/K7 (500.0 g, 2.58 mol, 1.0 equiv), DCM (3000 mL, 6 vol) and S2 (440.1 g, 2.71 mol, 1.05 equiv). The equipment was rinsed with additional DCM (500 mL, 1 vol), which was charged to the reactor. The mixture was cooled to 0° C. and methanesulfonic acid (1734 g, 18.04 mol, 7.0 equiv) was added over 2.5 h while maintaining an internal temperature below 15° C. The mixture was heated to 38° C. and stirred at that temperature for 16 hours at which time HPLC analysis indicated complete conversion to L1/K14. The mixture was cooled to 0° C. and the pH was adjusted to pH 10 with the addition of 4 M sodium hydroxide (3000 mL, 6 vol). The organic layer was separated and washed with water (2000 mL, 4.0 vol). The resulting organic phase was concentrated under reduced pressure to 3.5 volumes at 30° C. MTBE (5000 mL, 10 vol) was charged and the mixture was concentrated to 7.0 volumes under reduced pressure at 30° C. This put/take cycle was repeated two additional times. The resulting suspension was diluted with MTBE (1000 mL, 2 vol) to give a suspension of 9 total volumes. The suspension was heated to 50° C. for 2 hours, then charged with n-heptane (4500 mL, 9 vol) over 2 hours. The suspension was stirred at 50° C. for 12 h then cooled to 20° C. over 3 hours. After stirring at 20° C. for an additional 2 hours, the slurry was filtered, washed with 1:1 MTBE/n-heptane (1000 mL, 2.0 vol) and dried at 50° C. under vacuum with nitrogen stream for 16 hours to afford 700 g of L1/K14 (92% yield). A recrystallization was performed by suspending L1/K14 (8.2 kg) in a mixture of MTBE (41 L, 5 vol) and DCM (12 L, 1.5 vol) and heating the suspension to 55° C. n-Heptane (16 L, 2.0 vol) was added over 2 h. L1/K14 (0.05 wt %) seed crystals were charged and the resulting slurry was stirred for 1 h at 50° C. n-Heptane (25 L, 3.0 vol) was charged over 3 h and the mixture was stirred at 50° C. for an additional 1 hour. The mixture was cooled to 20° C. over 4 hours and stirred at that temperature for 16 hours. The solids were filtered, wash solids with 1:1 MTBE/n-heptane (16 L, 2 vol) and dried under vacuum with nitrogen bleed at 50° C. for 18 h to afford 6632 g of L1/K14. (81% yield from 8.2 kg of L1/K14).

Step 2: To a 20 L jacketed reactor was charged L1/K14 (600.0 g, 1775.14 mmol, 1.0 equiv), DCM (3600 mL, 6 vol) and triethylamine (299 mL, 2130.17 mmol, 1.2 equiv). The equipment was rinsed with additional DCM (600 mL, 1.0 vol), which was then added to the reactor. The mixture was cooled to 5° C. and trifluoroacetic anhydride (273 mL, 1952.65 mmol, 1.1 equiv) was added over 60 minutes while keeping the internal temperature below 15° C. The reaction mixture was warmed to 20° C. over 15 minutes, stirred for 2 hours and again cooled to 5° C. Water (2400 mL, 4.0 vol) was charged and the mixture was warmed to 23° C. and stirred for 30 minutes. After the phases were separated, the organic phase was washed sequentially with 2 M HCl (2×2400 mL, 2×4.0 vol), 1 M sodium carbonate (2400 mL, 4.0 vol) and water (2400 mL, 4.0 vol). The organic layer was then concentrated under reduced pressure to 3.5 volumes at 30° C. MTBE (4200 mL, 7 volumes) was charged and the mixture was concentrated to 7.0 volumes under reduced pressure. This put/take cycle was repeated four additional times. The resulting suspension was charged with MTBE (600 mL, 1 vol) and heated to 50° C. After stirring at 50° C. for 2 hours, n-heptane (4200 mL, 7.0 vol) was charged over 2 hours. The suspension was stirred for 12 hours at 50° C. then cooled to 20° C. over 3 hours. After stirring at 20° C. for an additional 2 hours, the suspension was filtered, washed with 1:1 MTBE/n-heptane (1200 mL, 2.0 vol) and dried at 50° C. under vacuum with nitrogen stream for 16 hours to afford 733 g of C154/K15 (95% yield).

Steps 3 and 4: To a 20 L jacketed reactor were charged C154/K15 (4651 g, 10.70 mol, 1.0 equiv), 1,3-Dibromo-5,5-dimethylhydantoin (2293.5 g, 8.02 mol, 0.75 equiv), chlorobenzene (18.6 L, 4.0 vol) and 1,4-dioxane (2.3 mL, 0.5 vol). The mixture was sparged with nitrogen for 30 minutes. In a separate flask, 2,2′-azo-bis-isobutyronitrile (AIBN, 228.3 g, 1.39 mol, 0.13 equiv) was dissolved in chlorobenzene (4.7 L, 1.0 vol) and the solution was sparge with nitrogen for 30 minutes. The suspension of C154/K15 was heated to 50° C. at which temperature the AIBN solution was added over 2 h. After 7.5 h, the conversion to K16 was complete and the mixture was cooled to 45° C. To the mixture at 45° C. was charged dimethylsulfoxide (pre-sparged with nitrogen for 30 minutes, 23.3 L, 5 vol) over 30 minutes followed by triethylamine (pre-sparged with nitrogen for 30 minutes, 4870.2 g, 6.7 L, 48.13 mol, 4.5 vol) over 30 minutes. The mixture was heated to 65° C. and stirred at that temperature for 14 h, at which time HPLC indicated complete conversion to S33/K17. The mixture was cooled to 20° C., charged with chlorobenzene (4.7 L, 1.0 vol) and DCM (32.6 L, 7.0 vol) then further cooled to 5° C. Water (35 L, 7.5 vol) was charged while keeping the internal temperature below 15° C. The mixture was warmed to 20° C., mixed and the layers separated. The organic phase was washed sequentially with 1.0 M HCl (35 L, 7.5 vol), water (37 L, 8.0 vol) and 5% sodium bicarbonate solution (37 L, 8.0 vol). The organic was concentrated under reduced pressure to 3.0 volumes at 35° C. Methanol (23.5 L, 5.0 vol) was added and the mixture was concentrated to 3.0 volumes under reduced pressure at 35° C. This put/take cycle was repeated four additional times. The resulting suspension was charged with methanol (9.3 L, 2 vol), heated to 50° C. and stirred for 5 hours. The suspension was cooled to 20° C. over 15 hours. The solids were filtered, washed with methanol (2.3 L, 0.5 vol) and dried at 50° C. under vacuum with nitrogen bleed for 16 hours to afford 2687 g of S33/K17 (56% yield). A recrystallization was performed by combining S33/K17 (8302 g), methanol (28 L, 3.3 vol) and acetone (14 L, 1.7 vol). The mixture was heated to 50° C. and stirred at that temperature for 5 hours. The suspension was cooled to 20° C. over 3 hours and stirred for an additional 15 hours. The solids were filtered, washed with 2:1 methanol/acetone (9 L, 1.0 vol) and methanol (8 L, 1 vol), then dried at 50° C. under vacuum with nitrogen bleed for 15 h to afford 6880.5 g of S33/K17 (83% yield from 8302 g of S33/K17).

Step 5. Method A: To a reactor was charged S33/K17 (6594.8 g, 14.69 mol, 1 equiv) follow by DCM (66 L, 10 vol). Stirring was started and the solution was cooled to 0° C. To a separate reactor was charged (pentamethylcyclopentadienyl)rhodium(III) dichloride dimer (4.5 g, 0.0073 mol, 0.0005 equiv), (1R,2R)-(−)-N-(4-toluenesulfonyl)-1,2-diphenylethylenediamine (5.4 g, 0.015 mol, 0.001 equiv) and DCM (6.6 L, 1.0 vol). The solution was stirred under nitrogen for 2 hours at 20° C. To a separate vessel was charged DCM (6.9 L, 1.05 vol), which was then sparged with nitrogen for 20 minutes and cooled to 0° C. To the cold DCM were then charged triethylamine (3716.9 g, 5120 mL, 36.73 mol, 2.5 equiv) followed by formic acid (1670.8 g, 1386 mL, 36.73 mol, 2.5 equiv) while keeping the internal temperature below 20° C. The resulting solution was cooled to 0° C. and stirred for 10 minutes. The previously prepared catalyst solution (at 20° C.) was charged to the triethylamine/formic acid solution (at 0° C.) over 30 minutes. The resulting mixture was stirred under nitrogen at 0° C. for 20 minutes, cooled to −5° C. then transferred to the reactor containing the stirring solution S33/K17 in dichloromethane at 0° C. over 30 minutes. A rinse of dichloromethane (2×6.6 L, 2×1 vol) was used to complete the transfer. The resulting golden yellow solution was stirred at 0° C. for 8 hours, then warmed to 10° C. over 3 hours and stirred at 10° C. for 13 hours. At that time, HPLC indicated 98.9% conversion to C63/K18. The reactor was charged with 3 M sodium chloride solution (40 L, 6.0 vol) followed by CPME (40 L, 6.0 vol). The layers were mixed for 30 minutes then separated. The organic phase was washed sequentially with 3.0 M sodium chloride solution (3×40 L, 3×6 vol), 0.6 M sodium bicarbonate solution (40 L, 6.0 vol), 3.0 M sodium chloride solution (40 L, 6 vol) and water (40 L, 6.0 vol). The organic phase was concentrated under reduced pressure to 3.0 volumes at 35° C. The resulting solution was charged with tetrahydrofuran (33 L, 5.0 vol) and SiliaMetS DMT resin (3.3 kg 50 wt % with respect to S33/K17). The resulting suspension was stirred at 20° C. for 16 hours. The suspension was filtered and the resin cake was washed with 1:2 CPME/THF (13.2 L, 2.0 vol). The filtrates and resin washes were combined and transferred back to the 2 L jacketed reactor. SiliaMetS DMT resin (3.3 kg 50 wt % with respect to S33/K17) was charged and the resulting suspension was stirred at 50° C. for 5 hours. The suspension was cooled to 20° C. over 30 minutes and stirred at 20° C. for an additional 12 hours. The suspension was filtered and the resin was washed with 1:2 CPME/THF (13.2 L, 2.0 vol). The filtrates were combined and concentrated to 3.0 volumes under reduced pressure at 35° C. CPME (33 L, 5.0 vol) was charged and the mixture was concentrated to 4.0 volumes under reduced pressure yielding a thin yellow slurry. Tetrahydrofuran (1319 g, 20 wt % with respect to S33/K17) was charged followed by C63/K18 seed crystals (3.8 g, 0.05 wt % with respect to S33/K17). The suspension was heated to 44° C. and stirred for 30 minutes at that temperature. n-Heptane (15.2 L, 2.3 vol) was charged over 2 hours at 42° C. yielding a thick suspension, which was stirred for an additional 30 minutes before heating to 50° C. After stirring at 50 C for 5 h, the slurry was cooled to 20° C. over 3 h and held at that temperature for an additional 5 hours. The solids were filtered, washed with 1:1 CPME/n-heptane (20 L, 3.0 vol). The solids were dried under vacuum with nitrogen stream at 50° C. for 12 hours to afford 6670.7 g (5516 g after potency correction) of C63/K18 as an CPME solvate (83.6% yield).

Step 5. Method B: S33/K17 (25.05 g, 53.02 mmol, 1 equiv) was charged to a 500 mL jacketed reactor, then diluted with DCM (200 mL, 8 vol) to partially dissolve the solids. (RhCl₂Cp*)₂ (16.5 mg, 0.0267 mmol, 0.0005 eq.) and (R,R)-TsDPEN (20.2 mg, 0.0551 mmol, 0.010 eq.) were added to the reactor, then rinsed in with DCM (50.0 mL, 2 vol). The mixture was cooled to 0° C. while sparging with N₂ for 30 min. In a separate 100 mL flask were charged DCM (30.0 mL, 1.2 vol) followed by TEA (18.47 mL, 132.5 mmol. 2.50 eq) and formic acid (5.00 mL, 132.5 mmol, 2.50 eq). After sparging was complete, the TEA/formic acid solution was added to the 500 mL reactor over 30 min at 0° C. to yield a clear golden/yellow solution. The reaction was stirred at 0° C. for 7 h, then warmed to 10° C. and stirred overnight. After stirring 28 h, HPLC analysis indicated >99% conversion to C63/K18. The reaction mixture was warmed to 20° C., diluted with CPME (150.0 mL, 6 vol) and quenched with 3 M brine (150.0 mL, 6 vol). The phases were mixed, settled and separated. The organic layer was washed with brine (3 M, 2×150.0 mL, 2×6 vol), followed by sodium bicarbonate (0.6 M, 150.0 mL, 6 vol), and water (150.0 mL, 6 vol). The organic phase was concentrated to 3 vol (˜75 mL). THF (125.0 mL, 5.0 vol) and Florisil (12.50 g, 50 wt %) were charged and the solution was stirred at 20° C. overnight. After 16 h the mixture was filtered and the collected solids were washed with 1:2 CPME/THF (50.0 mL, 2 vol). The filtrate and wash were combined. Florisil (12.50 g, 50 wt %) was charged to the solution and the resulting mixture was stirred under N₂ at 20° C. for 72 h. After 72 h the mixture was filtered and the collected solids were washed with 1:2 CPME/THF (50.0 mL, 2 vol). The filtrate and wash were combined and concentrated to 75 mL (3 vol) total volume. CPME (125 mL, 5 vol) was added and the solution was concentrated to 75 mL (3 vol). The put/take cycle was repeated a final time to yield a 75 mL (3 vol) slurry. CPME (25 mL, 1 vol) and THF (42.5 mL, 1.7 vol) were charged and the resulting mixture was heated to 65° C. The solution was cooled to 50° C. and seeded with C63/K18 THF solvate seed crystals (12.5 mg, 0.05 wt %). The mixture was stirred at 50° C. for 1 h, the n-heptane (57.5 mL, 2.3 vol) was charged over 2 h. After stirring an additional 5 h at 50° C., the slurry was further cooled to 20° C. and stirred overnight. The slurry was filtered and the isolated solids were washed with 1:1 CPME/heptane (3×25 mL, 3×1 vol). The solids were dried to provide 22.16 g of C63/K18 THF solvate (86% yield).

Step 6. Method A: To a reactor was charged C63/K18 (5299.4 g (potency adjusted), 11.75 mol, 1 equiv) and 2-propanol (12.5 L, 7 vol). The mixture was heated to 40-45° C., yielding a homogeneous solution. At 40° C., the solution was charged with 2 N sodium hydroxide (16.2 L, 35.26 mol, 3 equiv) over 20 minutes and the resulting cloudy mixture was stirred for 3 hours at which time HPLC analysis indicated full conversion to Compound II. The reaction mixture was cooled to 20° C. and charged sequentially with water (18.5 L, 3.5 vol), iPrOAc (34.4 L, 6.5 vol) and 2-propanol (4.8 L, 0.9 vol). After mixing for 30 min and separating the layers, the organic phase was washed sequentially with 10% NaCl (2×34.4 L, 2×6.5 vol) and water (20.7 L, 3.9 vol). The organic phase was concentrated under reduced pressure to 3 volumes. MEK (42 L, 8 vol) was charged and the mixture was concentrated to 3 volumes. This put/take cycle was repeated 3 additional times and the resulting mixture was diluted with MEK (7 vol) to give a 10-volume suspension. The slurry was heated to 78° C. and stirred at that temperature for 1 hour. The slurry was cooled to 65° C. over 30 minutes and seeded with Compound II (20.9 g, 0.059 mol, 0.005 equiv). The mixture was further agitated for 1 h at 65° C., then cooled to 20° C. over 12 hours. After stirring an additional 5 hours, the solids were filtered, washed with MEK (15.9 L, 3 vol) and dried at 50° C. under vacuum with nitrogen bleed to afford 2907.3 g of Compound II (69% yield). A recrystallization was performed by combining Compound II (2894.6 g) and MEK (57.9 L, 20 vol) and heating the suspension to 78° C. At 78° C., the solution was polish filtered, then cooled to 65° C. and seeded with Compound II (14.5 g, 0.04 mol, 0.005 equiv). The resulting suspension was stirred at 65° C. for 1 hour, then cooled to 20° C. over 12 hours. After stirring at 20° C. for an additional 5 h, the solids were filtered, washed with MEK (8.7 L, 3 vol) and dried under vacuum at 50° C. with nitrogen bleed for 19 hours to afford 2165.5 g of Compound II (75% yield from 2894.6 g of Compound II Form C).

Step 6. Method B: The C63/K18 THF solvate (18.4 g, 0.04084 mol, potency adjusted, 1 equiv) was charged to a 500 mL 3-neck round bottom flask and diluted with ethanol (46 mL, 2.5 vol). 3 M of sodium hydroxide in water (23.1 mL, 0.0693 mol, 1.7 eq) to give a slurry. The slurry was heated to 65° C., forming a clear solution, and stirred for 2 h. HPLC analysis at 2 h showed complete conversion to Compound II. The reaction solution was cooled to 50° C. over 30 minutes. Water (27.6 mL, 1.5 vol) was added to the reaction solution at 50° C. over 1 hour, maintaining a solution. The reaction solution was then seeded with Compound II Form C seed crystals (0.144 g) and stirred 1 h at 50° C. Water (225.3 mL, 12.2 vol) was then added to the reaction slurry over 1 hour creating a thin slurry with settling solids. The slurry was cooled to 20° C. temperature over 1 hour and stirred overnight. The slurry was filtered through a frit under vacuum. The reaction flask was rinsed with a pre-mixed solution of water:ethanol (6:1 v/v, 20 mL, 1.1 vol) and the rinse added to the solids in the frit and filtered. The solids in the frit were then rinsed sequentially with a pre-mixed solution of water: ethanol (6:1 v/v, 3×20 mL, 3×1.1 vol). The solids were dried further under vacuum filtration, then in a 50° C. vacuum oven for 8 h to provide 12.84 g of Compound II Form C (91% yield).

### Other Embodiments

This disclosure provides merely non-limiting exemplary embodiments of the disclosed subject matter. One skilled in the art will readily recognize from the disclosure and claims that various changes, modifications, and variations can be made therein without departing from the spirit and scope of the subject matter as defined in the following claims.

1. A solid form of
(a) Compound I selected from Compound I Phosphate Salt Hydrate Form A, Compound I free form Monohydrate, Compound I Phosphate Salt Methanol Solvate, Compound I Phosphate Salt MEK Solvate, Compound I Maleate Salt/Co-Crystal Form A, Compound I Maleate Salt/Co-Crystal Form B, Compound I Fumaric Acid Salt/Co-Crystal Form A, Compound I free form Form B, and Compound I free form Form C; or
(b) Compound II selected from Compound II Phosphate Salt Hemihydrate Form A, Compound II free form Hemihydrate Form A, Compound II free form Form A Compound II free form Form B, Compound II free form Form C, Compound II free form Quarter Hydrate, Compound II free form Hydrate Mixture, Compound II free form Monohydrate, Compound II free form Dihydrate, Compound II EtOH Solvate, Compound II free form IPA Solvate, Compound II free form MEK Solvate, Compound II free form MeOH Solvate, Compound II Phosphate Salt, Acetone Solvate, Compound II Phosphate Salt Form A, and Compound II Phosphate Salt Form C.

2.-32. (canceled)

33. A pharmaceutical composition comprising at least one solid form according to claim 1 and a pharmaceutically acceptable carrier.

34. A method of treating an APOL1 mediated disease comprising administering to a patient in need thereof at least one solid form according to claim 1 or a pharmaceutical composition according to claim 33.

35. The method according to claim 34, wherein the APOL1 mediated disease is an APOL1 mediated kidney disease.

36. The method according to claim 35, wherein the APOL1 mediated kidney disease is chosen from ESKD, NDKD, FSGS, HIV-associated nephropathy, arterionephrosclerosis, lupus nephritis, microalbuminuria, and chronic kidney disease.

37. The method according to claim 35, wherein the APOL1 mediated kidney disease is associated with one or two APOL1 genetic alleles chosen from homozygous G1: S342G:I384M and homozygous G2: N388del:Y389del.

38. The method according to claim 35, wherein the APOL1 mediated kidney disease is associated with compound heterozygous G1: S342G:I384M and G2: N388del:Y389del APOL1 genetic alleles.

39. The method according to claim 34, wherein the APOL1 mediated disease is cancer.

40. The method according to claim 34, wherein the APOL1 mediated disease is pancreatic cancer.

41. A method of inhibiting APOL1 activity comprising contacting said APOL1 with at least one solid form according to claim 1.

42. The method according to claim 41, wherein the APOL1 is associated with one or two APOL1 genetic alleles chosen from homozygous G1: S342G:I384M and homozygous G2: N388del:Y389del.

43. The method according to claim 41, wherein the APOL1 is associated with compound heterozygous G1: S342G:I384M and G2: N388del:Y389del APOL1 genetic alleles.

44. A method for preparing Compound I: 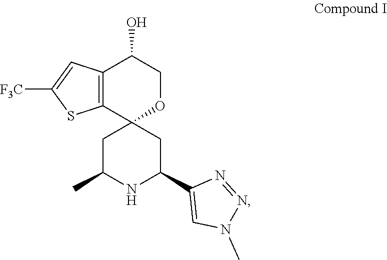 [SMILES: C[C@H]1C[C@@]2(C[C@@H](c3cn(C)nn3)N1)OC[C@@H](O)c1cc(C(F)(F)F)sc12]
comprising converting Compound C153/K13: 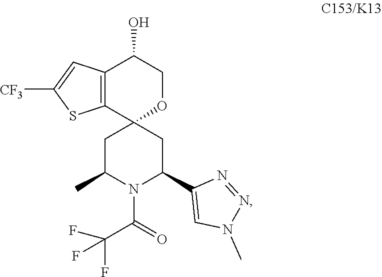 [SMILES: C[C@H]1C[C@@]2(C[C@@H](c3cn(C)nn3)N1C(=O)C(F)(F)F)OC[C@@H](O)c1cc(C(F)(F)F)sc12]
into Compound I.

45.-53. (canceled)

54. The method according to claim 44, wherein Compound C153/K13 is prepared by converting Compound S32/K12: 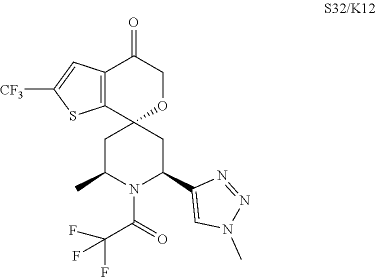 [SMILES: C[C@H]1C[C@@]2(C[C@@H](c3cn(C)nn3)N1C(=O)C(F)(F)F)OCC(=O)c1cc(C(F)(F)F)sc12]
into Compound C153/K13.

55.-56. (canceled)

57. The method according to claim 54, wherein Compound S32/K12 is prepared by converting Compound C62/K10: 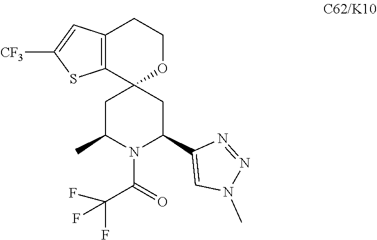 [SMILES: C[C@H]1C[C@@]2(C[C@@H](c3cn(C)nn3)N1C(=O)C(F)(F)F)OCCc1cc(C(F)(F)F)sc12]
into Compound S32/K12.

58. The method according to claim 57, wherein converting Compound C62/K10 into Compound S32/K12 comprises:
(i) converting Compound C62/K10 into Compound K11: 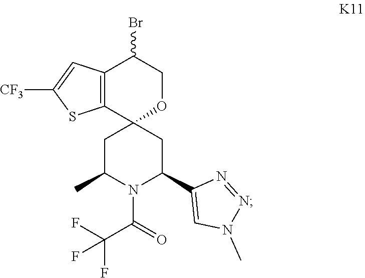
and
(ii) converting Compound K11 into Compound S32/K12.

59.-63. (canceled)

64. The method according to claim 57, wherein Compound C62/K10 is prepared by converting Compound L2/K9: 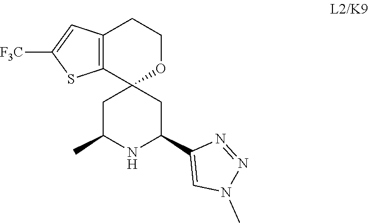 [SMILES: C[C@H]1C[C@@]2(C[C@@H](c3cn(C)nn3)N1)OCCc1cc(C(F)(F)F)sc12]
into Compound C62/K10.

65.-67. (canceled)

68. The method according to claim 64, wherein Compound L2/K9 is prepared by reacting Compound S26/K7: 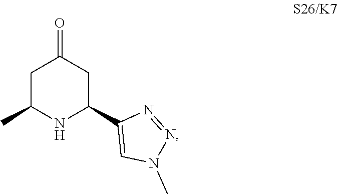
with Compound S3/J6/K8: 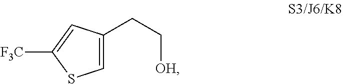 [SMILES: OCCc1csc(C(F)(F)F)c1]
to produce Compound L2/K9.

69.-71. (canceled)

72. A method of preparing Compound I comprising converting a compound selected from: 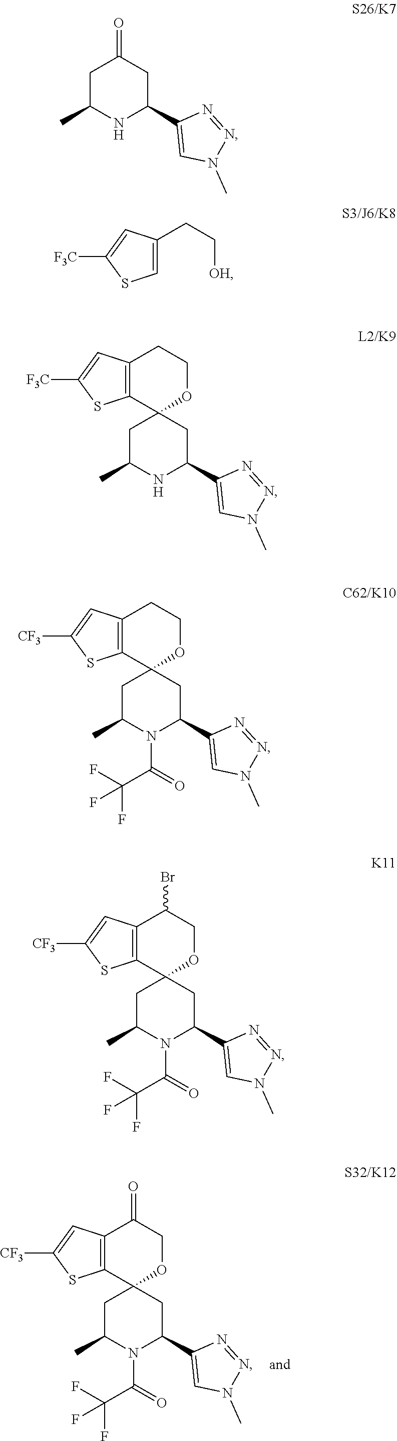 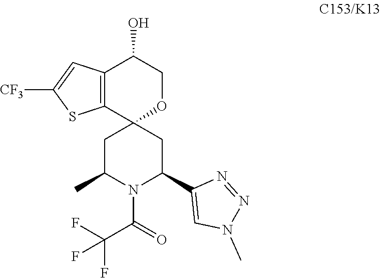 [SMILES: C[C@H]1C[C@@]2(C[C@@H](c3cn(C)nn3)N1C(=O)C(F)(F)F)OC[C@@H](O)c1cc(C(F)(F)F)sc12]
into Compound I.

73. A compound selected from:

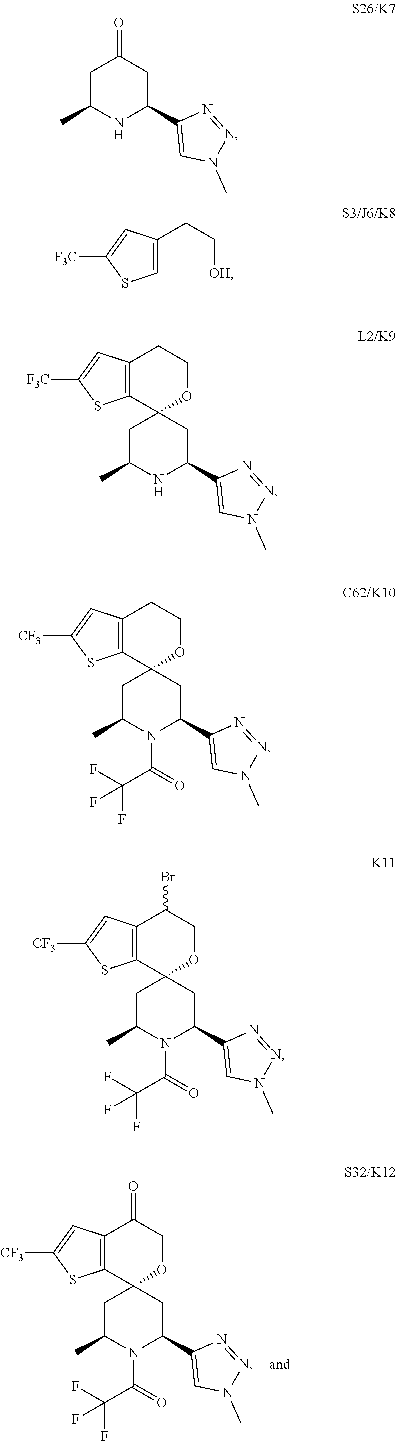

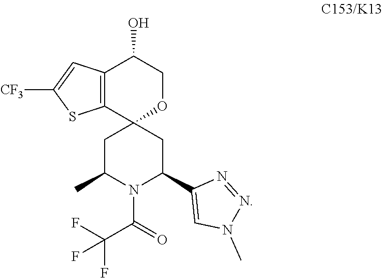 [SMILES: C[C@H]1C[C@@]2(C[C@@H](c3cn(C)nn3)N1C(=O)C(F)(F)F)OC[C@@H](O)c1cc(C(F)(F)F)sc12]

74. A method for preparing Compound II: 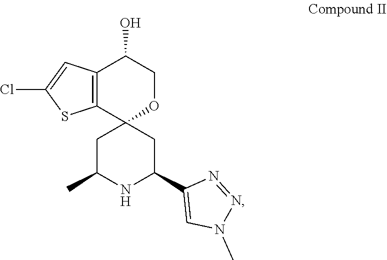 [SMILES: C[C@H]1C[C@@]2(C[C@@H](c3cn(C)nn3)N1)OC[C@@H](O)c1cc(Cl)sc12]
comprising converting Compound C63/K18: 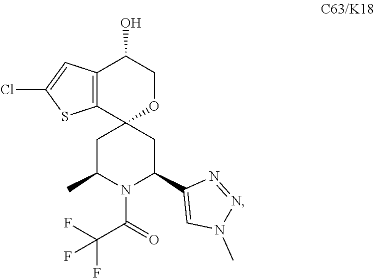 [SMILES: C[C@H]1C[C@@]2(C[C@@H](c3cn(C)nn3)N1C(=O)C(F)(F)F)OC[C@@H](O)c1cc(Cl)sc12]
into Compound II.

75.-79. (canceled)

80. The method according to claim 74, wherein Compound C63/K18 is prepared by converting Compound S33/K17: 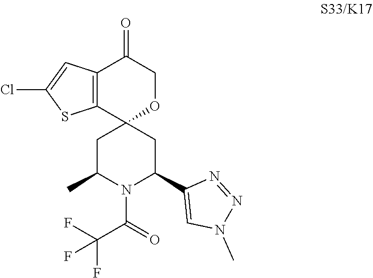 [SMILES: C[C@H]1C[C@@]2(C[C@@H](c3cn(C)nn3)N1C(=O)C(F)(F)F)OCC(=O)c1cc(Cl)sc12]
into Compound C63/K18.

81.-82. (canceled)

83. The method according to claim 80, wherein Compound S33/K17 is prepared by converting Compound C154/K15: 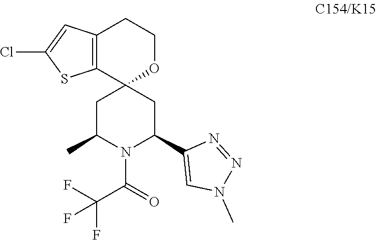 [SMILES: C[C@H]1C[C@@]2(C[C@@H](c3cn(C)nn3)N1C(=O)C(F)(F)F)OCCc1cc(Cl)sc12]
into Compound S33/K17.

84. The method according to claim 83, wherein converting Compound C154/K15 into Compound S33/K17 comprises:
(i) converting Compound C154/K15 into Compound K16: 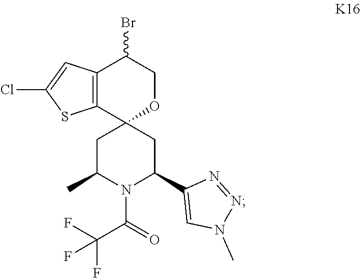
and
(ii) converting Compound K16 into Compound S33/K17.

85.-89. (canceled)

90. The method according to claim 83, wherein Compound C154/K15 is prepared by converting Compound L1/K14: 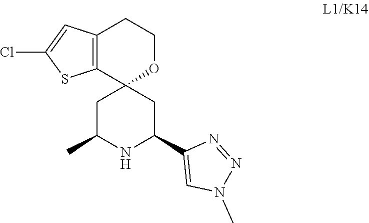 [SMILES: C[C@H]1C[C@@]2(C[C@@H](c3cn(C)nn3)N1)OCCc1cc(Cl)sc12]
into Compound C154/K15.

91.-93. (canceled)

94. The method according to claim 90, wherein Compound L1/K14 is prepared by reacting Compound S26/K7: 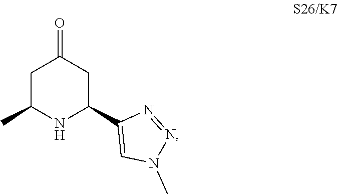
with Compound S2: 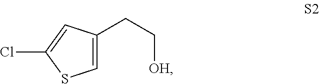
to produce Compound L1/K14.

95.-97. (canceled)

98. A method of preparing Compound II comprising converting a compound selected from: 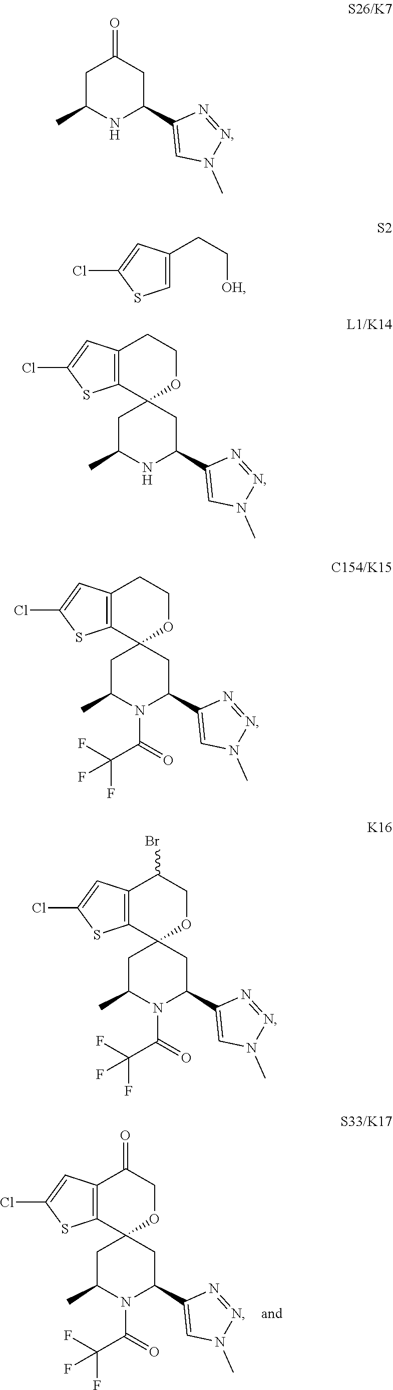 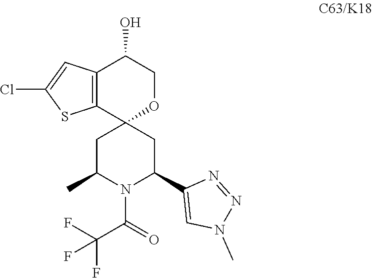 [SMILES: C[C@H]1C[C@@]2(C[C@@H](c3cn(C)nn3)N1C(=O)C(F)(F)F)OC[C@@H](O)c1cc(Cl)sc12]
into Compound II.

99. A compound selected from:

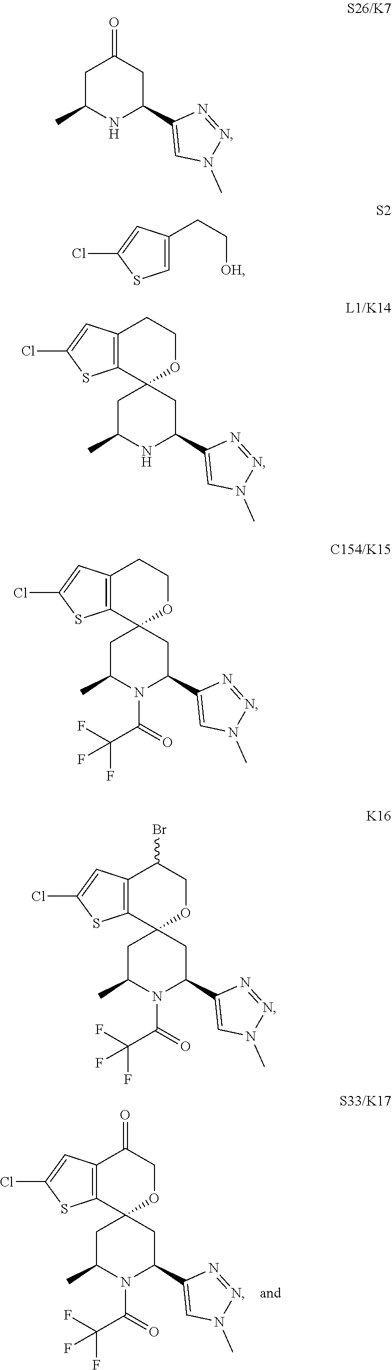

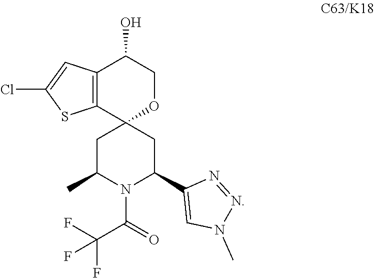 [SMILES: C[C@H]1C[C@@]2(C[C@@H](c3cn(C)nn3)N1C(=O)C(F)(F)F)OC[C@@H](O)c1cc(Cl)sc12]

100. A method for preparing Compound I: 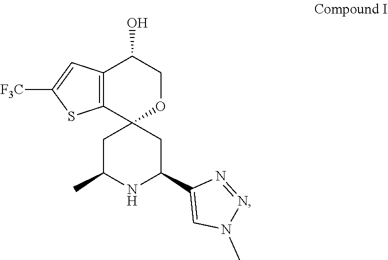 [SMILES: C[C@H]1C[C@@]2(C[C@@H](c3cn(C)nn3)N1)OC[C@@H](O)c1cc(C(F)(F)F)sc12]
comprising converting Compound 20a: 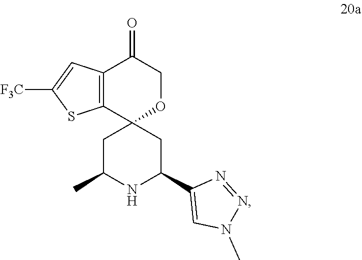 [SMILES: C[C@H]1C[C@@]2(C[C@@H](c3cn(C)nn3)N1)OCC(=O)c1cc(C(F)(F)F)sc12]
into Compound I.

101.-104. (canceled)

105. The method according to claim 100, wherein Compound 20a is prepared by converting Compound L2/K9: 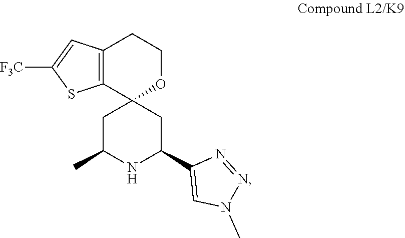 [SMILES: C[C@H]1C[C@@]2(C[C@@H](c3cn(C)nn3)N1)OCCc1cc(C(F)(F)F)sc12]
into Compound 20a.

106. (canceled)

107. The method according to claim 105, wherein Compound L2/K9 is prepared by reacting Compound S26/K7: 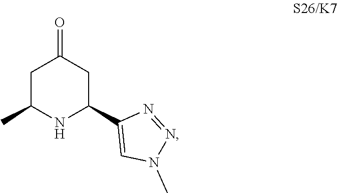
with Compound S3/J6/K8: 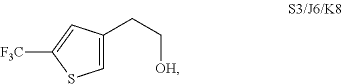 [SMILES: OCCc1csc(C(F)(F)F)c1]
to produce Compound L2/K9.

108.-109. (canceled)

110. A method for preparing Compound I comprising converting Compound 20a: 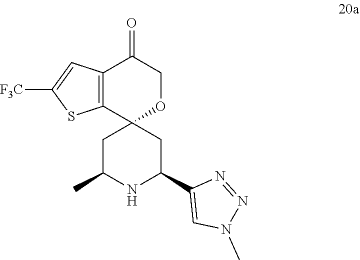 [SMILES: C[C@H]1C[C@@]2(C[C@@H](c3cn(C)nn3)N1)OCC(=O)c1cc(C(F)(F)F)sc12]
into Compound I.

111. Compound 20a:

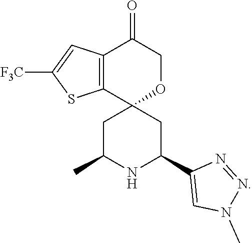 [SMILES: C[C@H]1C[C@@]2(C[C@@H](c3cn(C)nn3)N1)OCC(=O)c1cc(C(F)(F)F)sc12]

112. A method for preparing Compound II: 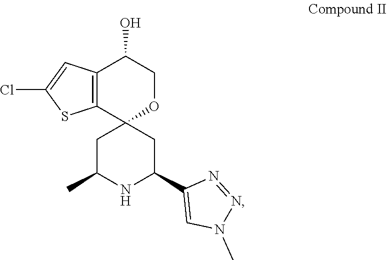 [SMILES: C[C@H]1C[C@@]2(C[C@@H](c3cn(C)nn3)N1)OC[C@@H](O)c1cc(Cl)sc12]
comprising converting Compound 20b: 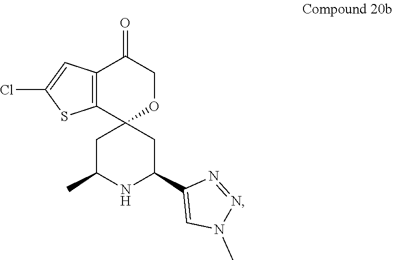 [SMILES: C[C@H]1C[C@@]2(C[C@@H](c3cn(C)nn3)N1)OCC(=O)c1cc(Cl)sc12]
into Compound II.

113.-114. (canceled)

115. The method according to claim 112, wherein Compound 20b is prepared by converting Compound L1/K14: 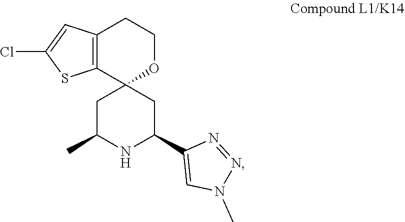 [SMILES: C[C@H]1C[C@@]2(C[C@@H](c3cn(C)nn3)N1)OCCc1cc(Cl)sc12]
into Compound 20b.

116. (canceled)

117. The method according to claim 115, wherein Compound L1/K14 is prepared by reacting Compound S26/K7: 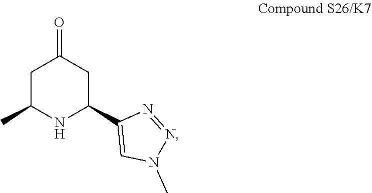
with Compound S2: 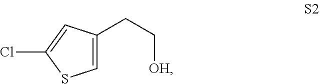
to produce Compound L1/K14.

118.-119. (canceled)

120. A method of preparing Compound II comprising converting Compound 20b: 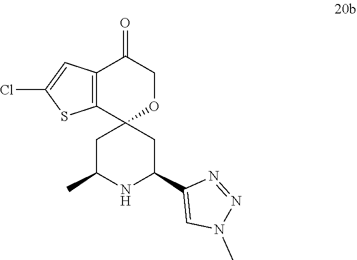 [SMILES: C[C@H]1C[C@@]2(C[C@@H](c3cn(C)nn3)N1)OCC(=O)c1cc(Cl)sc12]
into Compound I.

121. Compound 20b: 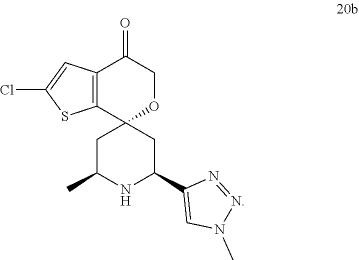 [SMILES: C[C@H]1C[C@@]2(C[C@@H](c3cn(C)nn3)N1)OCC(=O)c1cc(Cl)sc12]
